SEQUENCE LISTING (24 sequences)

SEQ ID NO: 1 - protein, 230 aa, Vibrio cholerae
MATSLPTPSAITTSALEYAFHTPARNLTKSRGKNIHRYVSVKMSKRITVESTLECDACYHFDFEPSIVRFCAQPIRFLYYLNGQSHSYVPDFLVQFDTNEFVLYEVKSAYAKNKPDFDVEWEAKVKAATELGLELELVEESDIRDTVVLNNLKRMHRYASKDELNNVHNSLLKIIKYNGAQSARCLGEQLGLKGRTVLPILCDLLSRCLLDTRLDKPLSLESRFELASYG

SEQ ID NO: 2 - protein, 603 aa, Vibrio cholerae
MAKKGFSSFHRKAVSSQDTLESIELVSSANCLESVTYQDISAFPETIAVEINFRLSILRFLARKCETIVAKSIEPHRVELQQNYSRKIPSAITIYRWWLAFRKSDYNPISLAPNIKDRGNRETKVSTVVDSIMEQAVERVISGRKVNVSSAYKRVRRKVRQYNLTHGTKYTYPKYESVRKRVKKKTPFELLAAGKGERVAKREFRRMGKKILTSSVLERVEIDHTVVDLFAVHEEYRIPLGRPWLTQLVDCYSKAVIGFYLGFEPPSYVSVSLALKNAIQRKDDLISSYESIENEWLCYGIPDLLVTDNGKEFLSKAFDQACESLLINVHQNKVETPDNKPHVERNYGTINTSLLDDLPGKSFSQYLQREGYDSVGEATLTLNEIREIYLIWLVDIYHKKPNQRGTNCPNVAWKKGCQEWEPEEFSGSKDELDFKFAIVDYKQLTKVGITVYKELSYSNDRLAEYRGKKGNHKVQFKYNPECMAVIWVLDEDMNEYFTVNAIDYEYASRVSLWQHKYNMKYQAELNSAEYDEDKEIDAEIKIEEIADRSIVKTNKIRARRRGARHQENSARAKSISNANPASIQKHEDEIVSADNDDWDIDYV

SEQ ID NO: 3 - protein, 330 aa, Vibrio cholerae
MSETREARISRAKRAFVSTPSVRKILSYMDRCRDLSDLESEPTCMMVYGASGVGKTTVIKKYLNQNRRESEAGGDIIPVLHIELPDNAKPVDAARELLVEMGDPLALYETDLARLTKRLTELIPAVGVKLIIIDEFQHLVEERSNRVLTQVGNWLKMILNKTKCPIVIFGMPYSKVVLQANSQLHGRFSIQVELRPFSYQGGRGVFKTFLEYLDKALPFEKQAGLANESLQKKLYAFSQGNMRSLRNLIYQASIEAIDNQHETITEEDFVFASKLTSGDKPNSWKNPFEEGVEVTEDMLRPPPKDIGWEDYLRHSTPRVSKPGRNKNFFE

SEQ ID NO: 4 - protein, 209 aa, Pseudoalteromonas sp.
MYIRNLRKPSPNKNVFKFASTKVSSVVMCESSLEFDACFHHEYNDLIESFGSQPEGFKYEFMGKSLPYTPDALISYTDKTQKYHEYKPYSKIASPLFRAEFAAKRAASLKLGIDLVLVTDRQIRVNPILNNLKLLHRYSGVYGISGIQKELLSFIHKSGVIKLNDISSQVGIPIGETRSFLFGLMHKGLVKADLGCDDLTNNPTLWATP

SEQ ID NO: 5 - protein, 613 aa, Pseudoalteromonas sp.
MTDFFNEFDESLVPLKPQTPTQYVKLDDANLIQRDLDTFSDTFKNQALQRYKLISTIDKKLSRGWTQRNLDPILDELFKGGDVVRPNWRTVARWRKKYIESNGDIASLADKNHKMGNRTNRIKGDDKFFDKALERFLDAKRPTIATAYQYYKDLIVIENESIVEGKIPIISYNAFNKRIKAIPPYAVAVARHGKFKADQWFAYCAAHVPPTRILERVEIDHTPLDLILLDDELLIPIGRPYLTLLIDVFSGCVLGFHLSYKSPSYVSAAKAITHAIKPKSLDALNIELQNDWPCFGKFENLVVDNGAEFWSKNLEHACQSAGINIQYNPVRKPWLKPFIERFFGVMNEYFLPELPGKTFSNILEKEEYKPEKDAIMRFSTFVEEFHRWIADVYHQDSNSRETRIPIKRWQQGFDAYPPLTMNEEEETRFSMLMRISDSRTLTRNGFKYQELMYDSTALADYRKHYPQTKETVKKLIKVDPDDISKIYVYLEELESYLEVPCTDPTGYTDGLSIYEHKTIKKINREVIRESKDSLGLAKARMAIHERVKQEQEVFIESKTKAKITAVKKQAQIADVSNTGTSTIKVSEESAAPVQKHISNDNSDDWDDDLEAFE

SEQ ID NO: 6 - protein, 333 aa, Pseudoalteromonas sp.
MNALTEIQIEKLRNFSDCIVMHPQIKTIFNDFDELRLNRKFQSDQQCMLLIGDTGVGKSHTINHYKKRVLATQNYSRNTMPVLVSRISRGKGLDATLVQMLADLELFGSSQIKKRGYKTDLTKKLVESLIKAQVELLIINEFQELIEFKSVQERQQIANGLKFISEEAKVPIVLVGMPWAAKIAEEPQWASRLVRKRKLEYFSLKNDSKYFRQYLMGLAKKMPFDVPPKLESKNTTIALFAACRGENRALKHLLLEALKLALSCNEYLENKHFITAYDKFDFFNDKEKLKSKNPFKQDIKDIEIYEVIKNSSYNPNALDPEDMLTDRVFAIVK

SEQ ID NO: 7 - protein, 394 aa, Vibrio cholerae
MFLQRPKPYSDESLESFFIRVANKNGYGDVHRFLEATKRFLQDIDHNGYQTFPTDITRINPYSAKNSSSARTASFLKLAQLTFNEPPELLGLAINRTNMKYSPSTSAVVRGAEVFPRSLLRTHSIPCCPLCLRENGYASYLWHFQGYEYCHSHNVPLITTCSCGKEFDYRVSGLKGICCKCKEPITLTSRENGHEAACTVSNWLAGHESKPLPNLPKSYRWGLVHWWMGIKDSEFDHFSFVQFFSNWPRSFHSIIEDEVEFNLEHAVVSTSELRLKDLLGRLFFGSIRLPERNLQHNIILGELLCYLENRLWQDKGLIANLKMNALEATVMLNCSLDQIASMVEQRILKPNRKSKPNSPLDVTDYLFHFGDIFCLWLAEFQSDEFNRSFYVSRW

SEQ ID NO: 8 - protein, 640 aa, Vibrio cholerae
MQTLKELIASNPDDLTTELKRAFRPLTPHIAIDGNELDALTILVNLTDKTDDQKDLLDRAKCKQKLRDEKWWASCINCVNYRQSHNPKFPDIRSEGVIRTQALGELPSFLLSSSKIPPYHWSYSHDSKYVNKSAFLTNEFCWDGEISCLGELLKDADHPLWNTLKKLGCSQKTCKAMAKQLADITLTTINVTLAPNYLTQISLPDSDTSYISLSPVASLSMQSHFHQRLQDENRHSAITRFSRTTNMGVTAMTCGGAFRMLKSGAKFSSPPHHRLNSKRSWLTSEHVQSLKQYQRLNKSLIPENSRIALRRKYKIELQNMVRSWFAMQDHTLDSNILIQHLNHDLSYLGATKRFAYDPAMTKLFTELLKRELSNSINNGEQHTNGSFLVLPNIRVCGATALSSPVTVGIPSLTAFFGFVHAFERNINRTTSSFRVESFAICVHQLHVEKRGLTAEFVEKGDGTISAPATRDDWQCDVVFSLILNTNFAQHIDQDTLVTSLPKRLARGSAKIAIDDFKHINSFSTLETAIESLPIEAGRWLSLYAQSNNNLSDLLAAMTEDHQLMASCVGYHLLEEPKDKPNSLRGYKHAIAECIIGLINSITFSSETDPNTIFWSLKNYQNYLVVQPRSINDETTDKSSL

SEQ ID NO: 9 - protein, 352 aa, Vibrio cholerae
MKLPTNLAYERSIDPSDVCFFVVWPDDRKTPLTYNSRTLLGQMEAASLAYDVSGQPIKSATAEALAQGNPHQVDFCHVPYGASHIECSFSVSFSSELRQPYKCNSSKVKQTLVQLVELYETKIGWTELATRYLMNICNGKWLWKNTRKAYCWNIVLTPWPWNGEKVGFEDIRTNYTSRQDFKNNKNWSAIVEMIKTAFSSTDGLAIFEVRATLHLPTNAMVRPSQVFTEKESGSKSKSKTQNSRVFQSTTIDGERSPILGAFKTGAAIATIDDWYPEATEPLRVGRFGVHREDVTCYRHPSTGKDFFSILQQAEHYIEVLSANKTPAQETINDMHFLMANLIKGGMFQHKGD

SEQ ID NO: 10 - protein, 199 aa, Vibrio cholerae
VKWYYKTITFLPELCNNESLAAKCLRVLHGFNYQYETRNIGVSFPLWCDATVGKKISFVSKNKIELDLLLKQHYFVQMEQLQYFHISNTVLVPEDCTYVSFRRCQSIDKLTAAGLARKIRRLEKRALSRGEQFDPSSFAQKEHTAIAHYHSLGESSKQTNRNFRLNIRMLSEQPREGNSIFSSYGLSNSENSFQPVPLI

SEQ ID NO: 11 - protein, 409 aa, Pseudoalteromonas sp.
MAFLFSPKARAFSDESLESYLLRVVSENFFDSYEGLSLAIREELHELDFEAHGAFPVDLKRLNVYHAKHNSHFRMRALGLLETLLDLPRYELQKLALLKSDIKFNSSVALYNNGVDIPLRFIRHHAEEAVDSIPVCSQCLAEEAYIKQSWHIKWVNACTKHQCALLHNCPECYAPINYIENESITHCSCGFELSCASTSPVNTLSIEHLNKLLDKGERNDSNPLFNNMTLTERFAALLWYQERYSQTDNFCLNDAVNYFSKWPAVFNTELDELSKNAEMKLIDLFNKTEFKFIFGDAILACPSTQKQSESHFIYRALLDYLVTLVESNPKTKKPNAADLLVSVLEAATLLGTSVEQVYRLYQNGILQTAFRHKMNQRINPYKGAFFLRHVIEYKTSFGNDKARMYLSAW

SEQ ID NO: 12 - protein, 695 aa, Pseudoalteromonas sp.
MHLKELLEITDTTERDRSLRRAFSPYTAMIDITGSEAVALIILLNLTYRKNQVDDLLDKKLAKQALKSEDHINKCIKEIAWFHTHNLKYPDIRVSKQNLAVEPPTLHSYVLSSANYPKAYGWSHNSAKVNFAKLFVSYFKWQNQVSWLAQVLATNSDNWKSAFTSLGLSVKAFKSLCVTVKNSLPEEAIPDSVDRYSRQIRMPYHDGYLAVTPVISHVVQSKIQQAAIDKRARFSNVEFTRPAAVSMLAASLGGVINVLNYPPYIRSKYHGLSNSRAFKLNNGQTVFNVEALLKPELIKALEGIIFSNNALALKQRRQQKVKNIKELRNTLLEWFSPVFEWRLDAIENGYDLEQLESASERLEYKILSLPDNELPSLTIPLFRLLNEMLGGVSMTQRYAFHPKLMSPLKAALQWLLVNLTDQKHVLIEEDDEHYRYLHLSGIRVFDAQALSNPYCSGIPSLTAVWGMIHSYQRKLNEALGTNVRFTSFSWFIRNYSAVAGKKLPELSLQGAQQSRLKRPGIIDGKYCDLVFDLIIHIDGYEDDLQAVDSKPDILKAHFPSNFAGGVMHQPELNSNINWCCLYSNENQLFEKLRRLPLSGCWVMPTEHKIQDLDELLLLLNSDSKLSPSMMGYMLLTEPMARVGSLERLHCYAEPAIGVVKYEAATSVRLKGIGNYFNSAFWMLDAQEKFMLMKKV

SEQ ID NO: 13 - protein, 350 aa, Pseudoalteromonas sp.
MELCNILKYDRSLYPGKAVFFYKTADSDFVPLEADINKIRGPKSGFTEAFTPQFSPKNISPQDLTHNNILTLEECYVPPNVEHIFCRFSLRVQANSLVPSGCSDPEVFSLLKELAETFKECGGYKELAVRYCRNILIGTWLWRNQNTGNTQIEIKTSKGSCYLIDNTRKLAWESKWASDDLKVLEELSNEIESALTDPNVFWSADITAKIEASFCQEIYPSQILNDKVKQGEASKQFVKAKCADGRYAVSFNSVKIGAALQSIDDWWDEDASKRLRVHEFGADKEIGVARRPPDSEQNFYSIFKNTEWYLSALKNCITNKNEKIDPAIYYLFSVLIKGGMFQKKAEAKKA

SEQ ID NO: 14 - protein, 203 aa, Pseudoalteromonas sp.
MQRYYFTVHFLPKQANLALLTGRCISIMHGFILKHNIEGMGVTFPAWSDSSIGNEIAFVYTDKEILNTLKDQAYFVDMQDCGFFKVSQVLAVPDSCEEVRFIRNQAVAKIFTGESRRRLKRLQKRALARGEDFNPKKIEAPREIDIFHRVAMTSKSSQEDYILHIQKQDVDCQAEPYFSNYGLASNEKFKGTVPDLSPSIDRN

SEQ ID NO: 15 - protein, 10 aa, synthetic construct
PKKKRKVEDP

SEQ ID NO: 16 - protein, 9 aa, synthetic construct
PAAKRVKLD

SEQ ID NO: 17 - protein, 16 aa, synthetic construct
KRPAATKKAGQAKKKK

SEQ ID NO: 18 - protein, 25 aa, synthetic construct
MSRRRKANPTKLSENAKKLAKEVEN

SEQ ID NO: 19 - protein, 18 aa, synthetic construct
KRTADGSEFESPKKKRKV

SEQ ID NO: 20 - DNA, 32 bp, synthetic construct
  source 1..32: mol_type = other DNA
taatggaaacttcctcatgaaaaagtctttag

SEQ ID NO: 21 - DNA, 30 bp, synthetic construct
  source 1..30: mol_type = other DNA
acagagagaataacataaaaacagggaagc

SEQ ID NO: 22 - DNA, 28 bp, synthetic construct
  source 1..28: mol_type = other DNA
gtgacctgccgtataggcagctgaaaat

SEQ ID NO: 23 - DNA, 28 bp, synthetic construct
  source 1..28: mol_type = other DNA
gtgacctgccgtataggcagctgaagat

SEQ ID NO: 24 - DNA, 27 bp, synthetic construct
  source 1..27: mol_type = other DNA
aattctgccgaaaaggcagtgagtagt

(19) United States
(12) Patent Application Publication
(10) Pub. No.: US 2025/0376701 A1
(43) Pub. Date: Dec. 11, 2025

(54) CRISPR-TRANSPOSON SYSTEMS AND COMPONENTS

(71) Applicants: The Trustees of Columbia University in the City of New York, New York, NY (US); The Broad Institute, Inc., Cambridge, MA (US); President and Fellows of Harvard College, Cambridge, MA (US)

(72) Inventors: Samuel H. Sternberg, New York, NY (US); Diego Gelsinger, New York, NY (US); George Davis Lampe, New York, NY (US); Rebeca Teresa King Davidson, New York, NY (US); David R. Liu, Cambridge, MA (US); Shannon Marie Miller, Cambridge, MA (US); Isaac Paterson Witte, Cambridge, MA (US); Simon Eitzinger, Cambridge, MA (US); Kiara N. Berrios Adorno, Cambridge, MA (US)

(21) Appl. No.: 19/300,168

(22) Filed: Aug. 14, 2025

Related U.S. Application Data
(63) Continuation of application No. PCT/US2024/015825, filed on Feb. 14, 2024.
(60) Provisional application No. 63/484,923, filed on Feb. 14, 2023.
(60) Provisional application No. 63/518,665, filed on Aug. 10, 2023.
(60) Provisional application No. 63/587,916, filed on Oct. 4, 2023.
(60) Provisional application No. 63/621,894, filed on Jan. 17, 2024.

(51) Int. Cl. C12N 15/90; C12N 9/22; C12N 15/11; C12N 15/70

(52) U.S. Cl. CPC C12N 15/907; C12N 9/22; C12N 15/11; C12N 15/70; C12N 2310/20; C12N 2800/90

(57) ABSTRACT

The present disclosure provides Clustered Regularly Interspaced Short Palindromic Repeats (CRISPR)-associated transposon (CRISPR-Tn or CAST) systems, components thereof, and methods for nucleic acid modification using the systems or components. More particularly, the disclosure provides modified Cas proteins and transposon-associated proteins for nucleic acid modification.

### CROSS-REFERENCE TO RELATED APPLICATIONS

This application is a continuation of International Application No. PCT/US2024/015825, filed Feb. 14, 2024, which claims the benefit of U.S. Provisional Application Nos. 63/484,923, filed Feb. 14, 2023, 63/518,665 filed Aug. 10, 2023, 63/587,916 filed Oct. 4, 2023, and 63/621,894, filed Jan. 17, 2024, the contents of each of which are herein incorporated by reference in their entirety.

### STATEMENT REGARDING FEDERALLY SPONSORED RESEARCH OR DEVELOPMENT

This invention was made with government support under HG011650, EB031935, HG009490, EB027793, EB031172, GM118062, and AI142756 awarded by the National Institutes of Health. The government has certain rights in the invention.

### FIELD

The present disclosure relates to Clustered Regularly Interspaced Short Palindromic Repeats (CRISPR)-associated transposon (CRISPR-Tn or CAST) systems and components thereof, for example, Cas proteins and transposon-associated proteins.

### SEQUENCE LISTING STATEMENT

The content of the electronic sequence listing titled COLUM-41261-601.xml (Size: 27,398 bytes; and Date of Creation: Feb. 14, 2024) is herein incorporated by reference in its entirety.

### BACKGROUND

In bacteria and archaea, CRISPR/Cas systems provide immunity by incorporating fragments of invading phage, virus, and plasmid DNA into CRISPR loci and using corresponding CRISPR RNAs (“crRNAs”) to guide the degradation of homologous sequences. Transcription of a CRISPR locus produces a “pre-crRNA,” which is processed to yield crRNAs containing spacer-repeat fragments that guide effector nuclease complexes to cleave dsDNA sequences complementary to the spacer. Several different types of CRISPR systems are known, (e.g., type I, type II, or type III), and classified based on the Cas protein type and the use of a proto-spacer-adjacent motif (PAM) for selection of proto-spacers in invading DNA.

Although RNA-guided targeting typically leads to endonucleolytic cleavage of the bound substrate, recent studies have uncovered a range of noncanonical pathways in which CRISPR protein-RNA effector complexes have been naturally repurposed for alternative functions. For example, some Type I (Cascade) and Type II (Cas9) systems leverage truncated guide RNAs to achieve potent transcriptional repression without cleavage and other Type I (Cascade) and Type V (Cas12) systems lie inside unusual bacterial Tn7-like transposons and lack nuclease components altogether.

### SUMMARY

Provided herein are engineered polypeptides, and nucleic acids encoding thereof, useful in Clustered Regularly Interspaced Short Palindromic Repeats (CRISPR)-associated transposon (CRISPR-Tn or CAST) systems and methods utilizing thereof. The polypeptides include transposon-associated proteins, such as TnsA, TnsB, TnsC, and TniQ, and Cas proteins, such as Cas5, Cas6, Cas7, and Cas8. The engineered proteins may show increased activity or utility in modifying a target nucleic acid. In some embodiments, the engineered proteins increase nucleic acid integration activity compared to a protein not having the disclosed modifications. In some embodiments, the engineered proteins increase or modify nucleic acid binding compared to a protein not having the disclosed modifications. In some embodiments, the engineered proteins increase nucleic acid integration activity or efficiency in vivo (e.g., in a prokaryotic or eukaryotic cell, in a subject) compared to a protein not having the disclosed modifications.

In some embodiments, the polypeptides comprise one or more amino acid sequences having at least 70% (e.g., at least 75%, at least 80%, at least 85%, at least 90%, at least 92%, at least 95%, at least 96%, at least 97%, at least 98%, or at least 99%) identity to any of SEQ ID NOs: 1-14 with one or more amino acid substitutions, deletions, or additions relative to SEQ ID NOs: 1-14.

In some embodiments, the polypeptide comprises an amino acid sequence having: at least 70% (e.g., having at least 75%, at least 80%, at least 85%, at least 90%, at least 92%, at least 95%, at least 96%, at least 97%, at least 98%, or at least 99%) identity to SEQ ID NO: 1 and one or more amino acid substitutions at positions: 2, 3, 5, 28, 57, 77, 80, 107, 110, 116, 122, 142, 155, 161, 166, 173, 177, 185, 211, 216, 227, and 230, relative to SEQ ID NO: 1; at least 70% (e.g., having at least 75%, at least 80%, at least 85%, at least 90%, at least 92%, at least 95%, at least 96%, at least 97%, at least 98%, or at least 99%) identity to SEQ ID NO: 2 and one or more amino acid substitutions at positions: 2, 5, 22, 24, 25, 29, 75, 141, 199, 215, 319, 347, 364, 370, 383, 439, 454, 458, 485, 509, 533, 538, 565, 581, 586, 595, 596, 597, and 600, relative to SEQ ID NO: 2; at least 70% (e.g., having at least 75%, at least 80%, at least 85%, at least 90%, at least 92%, at least 95%, at least 96%, at least 97%, at least 98%, or at least 99%) identity to SEQ ID NO: 3 and one or more amino acid substitutions at positions: 9, 15, 16, 18, 21, 64, 81, 86, 87, 99, 109, 142, 147, 153, 168, 180, 216, 230, 285, and 304, relative to SEQ ID NO: 3; at least 70% (e.g., having at least 75%, at least 80%, at least 85%, at least 90%, at least 92%, at least 95%, at least 96%, at least 97%, at least 98%, or at least 99%) identity to SEQ ID NO: 4 and one or more amino acid substitutions at positions: 4, 5, 9, 10, 12, 21, 23, 25, 26, 31, 32, 34, 35, 37, 41, 45, 47, 48, 51, 52, 55, 60, 61, 65, 67, 69, 72, 75, 79, 80, 82, 87, 88, 90, 91, 93, 94, 96, 98, 99, 100, 103, 106, 108, 113, 116, 125, 126, 128, 129, 135, 139, 143, 146, 147, 149, 153, 154, 156, 158, 159, 160, 162, 164, 166, 167, 168, 169, 170, 177, 179, 180, 182, 183, 185, 187, 188, 190, 191, 192, 193, 195, 196, 200, 204, 207, and 208, relative to SEQ ID NO: 4; at least 70% (e.g., having at least 75%, at least 80%, at least 85%, at least 90%, at least 92%, at least 95%, at least 96%, at least 97%, at least 98%, or at least 99%) identity to SEQ ID NO: 5 and one or more amino acid substitutions at positions: 1, 2, 4, 5, 8, 9, 10, 11, 12, 13, 14, 15, 16, 17, 19, 20, 21, 22, 23, 24, 25, 26, 27, 28, 29, 30, 32, 33, 36, 37, 39, 40, 41, 42, 43, 44, 45, 49, 52, 55, 56, 58, 60, 62, 63, 67, 71, 74, 76, 78, 79, 80, 81, 82, 83, 84, 85, 86, 87, 88, 89, 91, 92, 95, 97, 100, 101, 104, 106, 110, 112, 113, 115, 117, 119, 120, 124, 125, 127, 129, 130, 131, 134, 139, 142, 144, 145, 146, 147, 149, 150, 155, 156, 157, 158, 159, 163, 164, 165, 167, 169, 173, 174, 176, 181, 182, 186, 187, 190, 195, 197, 198, 205, 208, 209, 211, 215, 218, 223, 226, 227, 231, 232, 235, 239, 246, 248, 250, 259, 260, 261, 262, 263, 267, 269, 273, 274, 277, 278, 280, 281, 282, 283, 285, 287, 288, 290, 295, 298, 302, 303, 307, 313, 316, 317, 320, 323, 325, 331, 332, 339, 345, 348, 349, 352, 353, 354, 356, 361, 362, 363, 364, 365, 366, 367, 369, 370, 371, 372, 373, 375, 376, 380, 383, 385, 386, 389, 390, 392, 396, 397, 399, 402, 403, 404, 407, 408, 410, 411, 412, 413, 414, 415, 416, 421, 422, 423, 424, 425, 426, 427, 428, 429, 430, 431, 434, 435, 437, 440, 443, 445, 446, 448, 450, 452, 456, 459, 460, 463, 464, 470, 472, 473, 494, 495, 498, 501, 502, 504, 505, 506, 508, 509, 510, 512, 513, 514, 517, 520, 521, 522, 525, 526, 527, 530, 531, 532, 533, 535, 537, 538, 540, 541, 542, 543, 544, 545, 546, 547, 548, 549, 550, 551, 552, 553, 554, 556, 557, 558, 559, 560, 561, 562, 563, 564, 565, 567, 568, 569, 570, 571, 574, 575, 576, 580, 582, 583, 584, 585, 586, 587, 588, 589, 590, 591, 592, 593, 594, 595, 596, 597, 599, 600, 601, 602, 603, 604, 606, 607, 608, 611, 613, 618, 620, and 656, relative to SEQ ID NO: 5; at least 70% (e.g., having at least 75%, at least 80%, at least 85%, at least 90%, at least 92%, at least 95%, at least 96%, at least 97%, at least 98%, or at least 99%) identity to SEQ ID NO: 6 and one or more amino acid substitutions at positions: 1, 2, 3, 5, 6, 7, 9, 11, 12, 14, 21, 22, 26, 27, 31, 35, 38, 43, 44, 46, 47, 54, 59, 60, 61, 64, 65, 67, 68, 71, 72, 74, 76, 79, 80, 81, 84, 89, 95, 102, 105, 109, 110, 111, 112, 113, 114, 116, 118, 119, 120, 123, 129, 130, 131, 132, 134, 142, 145, 146, 147, 148, 150, 154, 155, 166, 169, 178, 180, 181, 183, 184, 187, 190, 194, 197, 201, 204, 207, 209, 213, 219, 221, 225, 226, 227, 229, 232, 233, 234, 236, 238, 241, 246, 251, 252, 256, 257, 261, 263, 265, 267, 269, 271, 272, 274, 280, 281, 285, 286, 288, 291, 292, 296, 299, 301, 303, 304, 306, 307, 308, 310, 313, 314, 316, 317, 318, 319, 320, 323, 324, 326, 328, 330, 331, 332, 340, 341, 343, 344, 355, 412, 418, 427, 514, 1198, 1201, 1206, 1212, 1260, and 1282, relative to SEQ ID NO: 6; at least 70% (e.g., having at least 75%, at least 80%, at least 85%, at least 90%, at least 92%, at least 95%, at least 96%, at least 97%, at least 98%, or at least 99%) identity to SEQ ID NO: 7 and one or more amino acid substitutions at positions: 99, 133, 189, 265, 266, 336, and 343, relative to SEQ ID NO: 7; at least 70% (e.g., having at least 75%, at least 80%, at least 85%, at least 90%, at least 92%, at least 95%, at least 96%, at least 97%, at least 98%, or at least 99%) identity to SEQ ID NO: 8 and one or more amino acid substitutions at positions: 119, 134, 155, 180, 183, 274, 319, 447, 454, 458, 461, 512, 538, and 580, relative to SEQ ID NO: 8; at least 70% (e.g., having at least 75%, at least 80%, at least 85%, at least 90%, at least 92%, at least 95%, at least 96%, at least 97%, at least 98%, or at least 99%) identity to SEQ ID NO: 9 and one or more amino acid substitutions at positions: 28, 82, 144, 151, 162, 182, 273, 327, and 346, relative to SEQ ID NO: 9; at least 70% (e.g., having at least 75%, at least 80%, at least 85%, at least 90%, at least 92%, at least 95%, at least 96%, at least 97%, at least 98%, or at least 99%) identity to SEQ ID NO: 10 and one or more amino acid substitutions at positions: 21 and 90, relative to SEQ ID NO: 10; at least 70% (e.g., having at least 75%, at least 80%, at least 85%, at least 90%, at least 92%, at least 95%, at least 96%, at least 97%, at least 98%, or at least 99%) identity to SEQ ID NO: 11 and one or more amino acid substitutions at positions: 2, 3, 7, 9, 11, 12, 14, 16, 20, 26, 29, 32, 34, 35, 40, 43, 45, 46, 54, 61, 64, 65, 70, 77, 101, 103, 105, 106, 108, 109, 111, 119, 120, 123, 126, 127, 130, 131, 148, 149, 151, 157, 159, 164, 166, 185, 194, 196, 203, 211, 217, 218, 219, 236, 242, 257, 267, 279, 283, 286, 288, 291, 293, 296, 303, 306, 313, 314, 316, 326, 331, 336, 347, 352, 361, 374, 377, 395, 396, 398, and 408, relative to SEQ ID NO: 11; at least 70% (e.g., having at least 75%, at least 80%, at least 85%, at least 90%, at least 92%, at least 95%, at least 96%, at least 97%, at least 98%, or at least 99%) identity to SEQ ID NO: 12 and one or more amino acid substitutions at positions: 4, 5, 6, 8, 9, 11, 12, 13, 16, 17, 20, 21, 24, 26, 28, 29, 34, 37, 38, 41, 49, 54, 59, 60, 63, 65, 67, 74, 77, 81, 88, 92, 93, 94, 96, 102, 105, 106, 108, 110, 121, 126, 128, 134, 138, 142, 147, 150, 151, 153, 156, 157, 160, 162, 165, 170, 171, 173, 174, 179, 181, 183, 185, 186, 187, 188, 191, 198, 201, 206, 207, 226, 228, 233, 236, 241, 249, 250, 256, 267, 268, 270, 275, 276, 277, 279, 283, 286, 289, 303, 305, 306, 310, 312, 314, 315, 316, 323, 326, 329, 349, 353, 355, 356, 357, 358, 361, 370, 372, 373, 376, 378, 382, 388, 391, 397, 399, 403, 405, 419, 421, 423, 424, 425, 427, 428, 430, 431, 432, 433, 449, 457, 473, 477, 480, 485, 487, 489, 494, 496, 497, 498, 500, 502, 509, 511, 515, 518, 519, 520, 540, 545, 550, 555, 557, 570, 571, 580, 583, 585, 590, 594, 603, 607, 608, 611, 617, 620, 624, 636, 639, 641, 642, 644, 646, 655, 658, 660, 663, 665, 668, 672, 673, 678, 682, 685, 688, and 695, relative to SEQ ID NO: 12; at least 70% (e.g., having at least 75%, at least 80%, at least 85%, at least 90%, at least 92%, at least 95%, at least 96%, at least 97%, at least 98%, or at least 99%) identity to SEQ ID NO: 13 and one or more amino acid substitutions at positions: 5, 10, 11, 26, 30, 35, 40, 42, 45, 46, 47, 58, 61, 65, 71, 72, 75, 77, 78, 80, 82, 83, 94, 98, 113, 115, 116, 117, 121, 128, 133, 138, 146, 148, 161, 171, 175, 177, 182, 184, 191, 193, 201, 203, 211, 212, 219, 225, 226, 232, 233, 235, 236, 237, 238, 240, 250, 274, 282, 286, 292, 295, 304, 307, 309, 312, 313, 315, 316, 317, 318, 320, 321, 322, 323, 328, 340, 343, 344, 345, 347, 348, 349, and 350, relative to SEQ ID NO: 13; or at least 70% (e.g., having at least 75%, at least 80%, at least 85%, at least 90%, at least 92%, at least 95%, at least 96%, at least 97%, at least 98%, or at least 99%) identity to SEQ ID NO: 14 and one or more amino acid substitutions at positions: 2, 9, 13, 14, 15, 34, 38, 42, 46, 50, 59, 60, 73, 75, 77, 82, 83, 85, 86, 97, 110, 115, 120, 124, 130, 132, 134, 140, 143, 145, 156, 159, 162, 164, 177, 199, 232, and 270, relative to SEQ ID NO: 14.

In some embodiments, the polypeptide comprises an amino acid sequence having: at least 70% (e.g., having at least 75%, at least 80%, at least 85%, at least 90%, at least 92%, at least 95%, at least 96%, at least 97%, at least 98%, or at least 99%) identity to SEQ ID NO: 1 and one or more amino acid substitutions of: A2T, T3I, L5S, T28A, A57T, F77L, Y80D, K107M, K107R, Y110C, Y110D, D116G, E122A, D142E, M155I, K161R, N166D, K173E, Y177N, Y177D, C185R, D211Y, K216E, A227P, G230D, and G230S, relative to SEQ ID NO: 1; at least 70% (e.g., having at least 75%, at least 80%, at least 85%, at least 90%, at least 92%, at least 95%, at least 96%, at least 97%, at least 98%, or at least 99%) identity to SEQ ID NO: 2 and one or more amino acid substitutions of: A2T, A2S, G5R, S22P, E24D, L25I, A29S, P75T, I141T, V199I, S215R, D319V, Y347F, S364N, E370K, N383D, V439A, E454D, E454G, S458N, V485F, R509G, D533A, A538V, H565Y, A581T, H586L, N595K, D596N, D597N, D597Y, and I600V, relative to SEQ ID NO: 2; at least 70% (e.g., having at least 75%, at least 80%, at least 85%, at least 90%, at least 92%, at least 95%, at least 96%, at least 97%, at least 98%, or at least 99%) identity to SEQ ID NO: 3 and one or more amino acid substitutions of: 19V, A15V, F16Y, S18F, S21N, N64D, H81Y, D86Y, N87K, V991, E109D, E142K, V147I, N153D, I168M, A180E, A216S, L230F, K285E, and R304R, relative to SEQ ID NO: 3; at least 70% (e.g., having at least 75%, at least 80%, at least 85%, at least 90%, at least 92%, at least 95%, at least 96%, at least 97%, at least 98%, or at least 99%) identity to SEQ ID NO: 4 and one or more amino acid substitutions of: R4K, N5K, P9S, A10P, N12D, T21I, V23M, S25N, S25R, V26M, V26G, S31N, S32I, E34A, F35L, A37D, H41L, D45N, I47V, E48G, G51V, S52I, E55K, E55D, E60K, F61L, S65T, S65A, P67T, P67L, P67S, P67H, T69A, A72V, A72D, S75I, S75R, S75T, K79E, T80P, K82E, K87R, P88L, P88T, P88A, S90F, K91N, K91E, A93T, A93S, S94N, L96P, R98Q, A99D, A99V, E100K, A103T, A106T, S108A, I113F, V116F, V116I, V125M, V125A, N126T, I128V, I128L, L129P, L135M, S139N, S139G, G143V, G143C, G146D, G146S, I147V, K149E, K149T, K149R, S153I, S153R, S153N, F154C, H156R, H156L, S158N, S158R, G159V, V160A, K162R, N164D, I166L, S167I, S168I, S168R, S168N, Q169R, V170M, V170G, V170L, T177I, T177A, S179R, F180C, F180L, F182C, F182L, G183S, M185I, K187R, G188D, V190I, K191N, A192S, D193N, G195V, G195D, G195S, C196W, T200A, T204I, A207V, A207T, and T208I, relative to SEQ ID NO: 4; at least 70% (e.g., having at least 75%, at least 80%, at least 85%, at least 90%, at least 92%, at least 95%, at least 96%, at least 97%, at least 98%, or at least 99%) identity to SEQ ID NO: 5 and one or more amino acid substitutions of: M1V, M1I, M1L, T2I, T2A, F4L, F5L, F8L, F8V, F8S, D9N, E10K, E10D, S11I, S11R, S11G, L12P, V13M, V13G, V13E, V13L, P14L, L15Q, K16N, K16R, P17T, P17L, P17S, T19I, T19S, T19A, T19P, P20S, P20L, T21A, Q22R, Y23H, V24M, K25R, L26M, D27A, D27G, D28N, D28Y, A29T, A29V, N30K, I32F, I32S, Q33H, L36M, D37A, D37Y, F39L, S40P, D41E, T42I, T42K, T42A, F43L, F43S, F43V, K44N, N45D, N45S, Q49R, K52Q, S55A, T56A, D58E, K60Q, S62T, R63K, R63G, Q67R, Q67H, Q67K, D71Y, K74R, E76K, F78C, K79R, G80V, G80D, G81S, G81V, G81D, D82N, V83G, V83M, V83A, V84A, V84G, R85G, R85K, P86L, N87S, R89C, V91G, V91A, A92V, A92T, R95K, K97R, E100D, S101A, D104V, A106D, A106T, D110N, N112H, H113Y, M115R, N117Y, T119A, N120D, N120K, N120S, G124V, D125N, D125E, K127R, F129L, D130N, K131M, E134D, E134G, A139S, A139T, P142S, I144V, A145S, A145T, T146A, A147V, Q149R, Y150H, I155L, V156A, V156L, V156M, K157V, E158A, N159S, V163G, E164A, E164G, E164D, G165D, I167V, I169L, I169T, N173S, N173H, N173T, A174S, A174T, N176D, A181S, I182L, I182V, I182T, A186E, A186T, V187G, V187A, A190T, A190S, F195S, A197P, D198G, D198N, A205S, V208M, P209T, T211I, E215D, E218D, P223S, P223H, L226V, I227V, D231N, E232K, I235V, I235T, R239G, I246V, V248E, V248M, S250I, S259N, Y260C, K261R, S262N, P263L, S267N, A269V, T273I, T273N, H274Y, K277N, K277R, P278S, S280T, L281M, D282E, D282N, A283T, A283S, N285S, E287D, L288M, N290K, F295S, F298I, F298S, V302I, V303M, A307S, N313S, H316R, A317V, S320N, S320R, I323L, I325V, R331K, K332E, I339V, V345L, V345M, E348K, Y349H, Y349D, Y349N, Y349C, P352S, P352T, E353Q, E353D, L354M, G356S, N361D, I362V, I362T, L363P, L363T, L363M, E364G, K365R, E366G, E367G, K369N, K369E, K369M, P370S, E371K, V372M, D373G, I375V, M376I, T380P, T380A, E383K, E383D, F385L, H386Y, I389V, A390V, A390I, V392I, D396N, D396G, D396K, S397P, S399N, S399G, T402I, R403G, R403I, R403K, R403S, I404T, I404V, K407R, K407E, R408K, Q410K, Q410H, Q410R, Q411H, G412V, F413L, D414N, A415V, A415T, Y416C, M421I, N422K, E423K, E423D, E424A, E425K, E426D, T427A, T427S, R428K, F429L, S430A, M431L, R434H, R434C, R434S, I435V, D437G, D437N, T440S, T440I, R443C, G445S, F446L, F446I, Y448C, E450D, E450G, M452I, T456P, T456A, T456I, A459T, D460N, K463N, H464N, H464R, H464S, E470K, V472M, V472A, K473D, K473N, E494D, E494G, S495A, E498A, E498K, C501Y, T502I, T502S, P504S, P504L, T505A, G506Y, G506D, G506L, G506S, T508A, D509E, D509Y, C510Y, S512N, I513L, I513V, I513F, Y514H, K517M, K517N, K517Q, K520R, K521N, I522T, I522V, I522F, E525K, V526E, V526M, I527V, S530N, S530R, K531T, D532G, D532Y, S533Y, G535D, A537T, K538R, K538N, R540K, R540G, M541L, A542T, I543L, H544R, E545A, R546G, R546K, V547M, K548Q, K548R, Q549K, Q549R, E550A, Q551K, E552D, E552K, V553I, F554V, E556K, E556G, S557A, K558R, T559P, T559I, T559A, K560R, A561T, A561G, K562R, K562N, I563L, T564I, A565S, A565V, K567R, K568N, K568R, Q569K, Q569L, Q569R, A570V, Q571R, D574N, V575M, V575A, S576R, T580I, T580A, T582I, T582S, I583V, K584R, V585M, S586P, S586A, S586F, E587A, E588K, E588G, E588D, S589I, S589R, S589N, A590S, A590T, A591V, P592L, V593M, V593A, Q594L, K595R, K595N, H596Y, H596L, H596P, I597T, I597V, N599H, D600L, D600N, D600G, D600V, N601S, N601K, S602A, S602P, S602Y, D603A, D603V, D604G, D604Y, D604N, D606A, D606V, D606Y, D607Y, D607E, D608N, A611T, E613D, R618I, T620P, and A656V, relative to SEQ ID NO: 5; at least 70% (e.g., having at least 75%, at least 80%, at least 85%, at least 90%, at least 92%, at least 95%, at least 96%, at least 97%, at least 98%, or at least 99%) identity to SEQ ID NO: 6, and one or more amino acid substitutions of: M1L, M1V, N2S, A3T, T5P, T5A, T5S, E6D, 17S, 17V, 19F, Q11R, L12M, N14D, N14S, M21I, H22P, H22Y, K26N, K26R, T27I, M31I, L35R, N38S, S43P, D44N, D44G, Q46L, C47S, T54I, S59T, H60Y, T61A, H64Y, Y65H, K67N, K67R, R68Q, A71G, T72A, N74D, S76C, S76Y, T79I, M80I, P81S, V84L, R89L, A95D, A95T, A102T, E105D, E105K, S109N, S109R, S110P, Q111R, I112T, K113N, K113E, K114N, K114M, K114E, G116D, K118N, K118R, T119I, D120V, K123N, L129M, I130V, K131R, A132S, K134M, K134N, F142V, L145M, I146T, E147K, F148S, S150F, R154K, Q155H, E166D, K169E, P178S, A180V, A181T, A181S, I183V, A184S, A184T, A184V, P187S, A190T, A190V, V194M, V194A, R197I, Y201N, L204M, D207N, K209N, Q213H, Q213V, A219S, K221N, D225N, V226E, P227T, K229E, S232N, K233N, K233R, N234H, T236A, A238V, A238S, A241S, E246D, K251N, H252Y, H252R, E256D, A257S, A261V, S263I, S263N, N265D, Y267C, E269K, E269D, K271E, K271R, H272Y, I274V, F280L, D281N, D281G, K285G, K286N, K288R, S291F, S291P, K292N, K296R, K296N, I299S, D301G, E303D, I304T, I304V, E306G, V307L, V307G, V307A, V307D, V307G, I308N, N310S, Y313H, N314K, N316K, N316D, A317D, L318Q, D319N, P320S, P320L, M323I, L324M, D326N, V328M, V328A, A330D, I331V, V332G, S340L, T341A, A343G, S344N, I355V, F412V, V418F, Y427C, R514K, S1198L, A1201V, G1206S, C1212G, F1260L, and V1282M, relative to SEQ ID NO: 6; at least 70% (e.g., having at least 75%, at least 80%, at least 85%, at least 90%, at least 92%, at least 95%, at least 96%, at least 97%, at least 98%, or at least 99%) identity to SEQ ID NO: 7, and one or more amino acid substitutions of: M99I, S189N, H265Q, A266V, L336F, and V343A, relative to SEQ ID NO: 7; at least 70% (e.g., having at least 75%, at least 80%, at least 85%, at least 90%, at least 92%, at least 95%, at least 96%, at least 97%, at least 98%, or at least 99%) identity to SEQ ID NO: 8 and one or more amino acid substitutions of: Y119H, N134R, N134Q, D155N, Q180R, D183N, R274L, N319D, V447I, A454S, E458G, D461N, A512T, D538K, and P580Q, relative to SEQ ID NO: 8; at least 70% (e.g., having at least 75%, at least 80%, at least 85%, at least 90%, at least 92%, at least 95%, at least 96%, at least 97%, at least 98%, or at least 99%) identity to SEQ ID NO: 9 and one or more amino acid substitutions of: R28K, A82T, K144E, C151R, N162S, K182E, D273G, A327D, and M346I, relative to SEQ ID NO: 9; at least 70% (e.g., having at least 75%, at least 80%, at least 85%, at least 90%, at least 92%, at least 95%, at least 96%, at least 97%, at least 98%, or at least 99%) identity to SEQ ID NO: 10 and one or more amino acid substitutions of: A21S and V90A, relative to SEQ ID NO: 10; at least 70% (e.g., having at least 75%, at least 80%, at least 85%, at least 90%, at least 92%, at least 95%, at least 96%, at least 97%, at least 98%, or at least 99%) identity to SEQ ID NO: 11 and one or more amino acid substitutions of: A2T, F3S, P7R, A9S, A9G, A11G, F12I, D14N, S16Y, Y20H, S26N, F29S, S32N, E34K, G35V, G35S, G35D, I40S, E43D, H45P, E46K, A54S, R61W, V64M, Y65C, N70S, A77T, D101N, K103E, N105K, N105D, S106G, V108M, A109G, Y111N, L119M, R120S, R123S, A126T, E127G, V130M, D131N, Q148R, S149Y, H151Y, A157D, T159I, A164V, L166M, T185A, S194G, A196T, T203A, K211R, E217K, R218K, R218S, N219S, A236T, E242D, N257K, N267S, M279I, M279V, D283G, N286S, T288I, K291Q, I293V, D296N, S303I, S303G, K306N, S310Y, S310P, I313T, Y314F, A316T, E326G, T331I, A336V, A347T, A347S, T352S, Y361H, M374T, M374I, R377G, T395I, S396T, S396F, G398V, and A408V, relative to SEQ ID NO: 11; at least 70% (e.g., having at least 75%, at least 80%, at least 85%, at least 90%, at least 92%, at least 95%, at least 96%, at least 97%, at least 98%, or at least 99%) identity to SEQ ID NO: 12 and one or more amino acid substitutions of: K4N, E5K, L6M, L6I, E8K, E8D, 19T, D11N, T12A, T13I, D16G, R17C, R17S, R20K, R20E, R21E, R21K, S24K, S24Q, S24R, Y26S, Y26H, A28S, A28D, M29I, G34D, A37S, V38M, V38G, I41V, R49L, D54G, K59R, K60N, K63N, A65T, A65V, K67E, K74E, K77E, W81C, K88R, K88E, 192T, R93E, R93K, V94M, K96N, E102D, E102G, T105A, L106M, S108P, V110A, G121S, S126P, K128R, L134M, Y138S, Q142H, W147L, K150N, V151M, V151L, A153T, S156R, S156G, D157N, K160R, K160E, A162T, S165N, S165G, V170E, K171E, F173V, K174N, K174R, T179A, K181T, S183N, P185T, E186K, E186D, E187K, A188S, A188V, D191Y, D191E, R198H, R198C, R198S, R201K, D206G, G207D, A226T, I228V, R233K, N236T, R241E, A249S, A250S, I256T, S267G, S267N, K268N, H270P, S275N, S275G, R276G, A277D, A277S, A277T, K279N, G283D, V286G, V289M, G303D, I305T, F306S, A310D, A310T, A312G, A312D, A312T, K314N, Q315R, R316G, N323S, E326A, E326K, N329S, G349D, E353D, L355M, L355R, E356G, E356D, S357P, A358V, R361S, P370T, N372K, E373D, S376F, T378I, F382L, M388V, G391S, R397K, A399S, K403N, M405I, L419P, D421N, K423R, H424N, H424R, V425L, I427V, E428K, D430A, D431G, E432D, H433N, A449T, G457D, R473K, E477D, G480D, F485L, S487R, S487G, S489N, N494D, S496N, A497S, V498G, K500N, K502N, Q509R, A511T, A511E, R515S, R518S, P519T, G520D, G520V, Y540C, Q545H, K550N, K555E, H557Q, P570S, E571D, C580R, S583R, E585K, E585G, E590D, R594K, M603I, H607N, H607L, K608R, D611N, L617P, N620S, K624N, T636P, M639V, N641S, V642G, S644N, S644G, E646D, A655V, V658M, K660N, T663A, T665I, R668S, I672V, G673V, S678R, M682L, A685V, A685D, K688N, and V695M, relative to SEQ ID NO: 12; at least 70% (e.g., having at least 75%, at least 80%, at least 85%, at least 90%, at least 92%, at least 95%, at least 96%, at least 97%, at least 98%, or at least 99%) identity to SEQ ID NO: 13 and one or more amino acid substitutions of: N5K, N5T, D10N, R11K, D26N, V30E, D35N, R40L, P42A, G45S, G45V, F46V, T47R, T47S, N58T, P61L, T65I, T71I, T71R, T71D, L72M, C75S, V77A, P78L, N80T, E82D, H83Y, H83N, A94S, V98M, E113D, C121F, A128S, A155S, E116D, T117I, R133K, G138V, N146D, G148V, C161R, A171V, A171S, K175T, A177V, K182E, L184M, I191V, S193A, S193F, F201S, S203N, E211K, A212V, Y219R, N225S, N225T, D226Y, E232K, E232Q, A233N, A233S, A233K, K235R, Q236R, Q236S, F237L, V238Q, V238M, A240T, A240V, S250A, R274G, A282V, I286N, I286T, I286F, P292S, S295N, K304R, E307D, Y309C, A312V, L313M, N315K, N315T, N315S, C316G, I317V, T318A, T318P, K320R, N321D, E322K, K323N, I328T, M340I, K343E, K343R, K344E, K344R, A345T, A345D, A345S, A345Y, A345R, A345K, A345E, A345G, A347K, A347S, A347D, K348N, K349R, A350K, A350D, A350V, and A350T, relative to SEQ ID NO: 13; or at least 70% (e.g., having at least 75%, at least 80%, at least 85%, at least 90%, at least 92%, at least 95%, at least 96%, at least 97%, at least 98%, or at least 99%) identity to SEQ ID NO: 14 and one or more amino acid substitutions of: Q2K, H9L, K13E, Q14K, A15G, K34N, E38K, V42I, A46D, S50I, V59G, Y60H, A73S, A73T, F75L, D77G, G82S, F83L, F83V, F83C, K85E, V86I, E97, I110S, I110L, S115R, K120N, K124R, G130D, D132E, N134T, A140T, E143K, D145G, S156I, E159K, I162V, H164Y, H164F, Y177C, S199I, S232L, and L270S, relative to SEQ ID NO: 14.

In some embodiments, the polypeptide comprises an amino acid sequence having: at least 70% (e.g., having at least 75%, at least 80%, at least 85%, at least 90%, at least 92%, at least 95%, at least 96%, at least 97%, at least 98%, or at least 99%) identity to SEQ ID NO: 1 and amino acid substitutions at positions: 2 and 230; 107 and 166; 107, 166, and one or both of: 2 and 227; 211 and 110 or 142; 110, 155 and 230; 122 and 155; or 155 and 177, relative to SEQ ID NO: 1; at least 70% (e.g., having at least 75%, at least 80%, at least 85%, at least 90%, at least 92%, at least 95%, at least 96%, at least 97%, at least 98%, or at least 99%) identity to SEQ ID NO: 2 and amino acid substitutions at positions: 2 and 597; 24 and 25; 24, 25, 458, 509, 565, and 600; 75 and 597; 141, 454, 533 and 595; 581, 370, and 454; 370 and 581; 370 and 454; 458 and 509; 458, 509 and 565; 458, 509, 565, and 600; 565, 586, and 596; or 565, 509, 458, 600, and at least one of 24, 25, 29, 215, 319, 364, 383, and 586, relative to SEQ ID NO: 2; at least 70% (e.g., having at least 75%, at least 80%, at least 85%, at least 90%, at least 92%, at least 95%, at least 96%, at least 97%, at least 98%, or at least 99%) identity to SEQ ID NO: 3 and amino acid substitutions at positions: 142 and 216, relative to SEQ ID NO: 3; at least 70% (e.g., having at least 75%, at least 80%, at least 85%, at least 90%, at least 92%, at least 95%, at least 96%, at least 97%, at least 98%, or at least 99%) identity to SEQ ID NO: 4 and amino acid substitutions at positions: 108 and 47 or 208; 170 and 207; 88 and 147; 47, 88 and 147; 88, 147, 170, and 182; 88, 147, 170, 182, and 51 or 180; 88, 147, and 154; 88, 128, 147, 170, and 182; or 170, 207, and 108, relative to SEQ ID NO: 4; at least 70% (e.g., having at least 75%, at least 80%, at least 85%, at least 90%, at least 92%, at least 95%, at least 96%, at least 97%, at least 98%, or at least 99%) identity to SEQ ID NO: 5 and amino acid substitutions at positions: 4, 23 and 590; 19, 169, and 549; 43 and 415; 80 and 593; 80, 144, 593, and 606; 1, 42, 80, 593, and 606; 42, 80, 593, and 606; 156 and 604; 283, 349, and 365; 283, 349, 365, 396, and 594; 283, 349, 365, 396, 594, 596, and 131; 352 and 390; 390, 396, and 594; 396 and 594; 456 and 502; 464 and 502; 464 and 17; 17, 235, 464, and 596; 235, 352, 396, 456, and 606; 415, 456, and 502; 456, 502, and 549; 169, 456, 502, and 549; 80, 456, 502, 593, and 606; 1, 42, 80, 456, 502, 593, and 606; 80, 144, 456, 502, 593, and 606; 19, 169, 456, 502 and 549; 43, 415, 456, and 502; 352, 390, 396, and 594; 352, 390, and 396; 283, 349, 396, and 594; 11, 55, 120, 362, 584, 600, and 604; 43, 84, 144, 349, and 517; 164 and 165; 164 and 173; 362 and 446; 352, 390, 396, 549, and 594; 352, 390, 396, 464, 549, and 594; 43, 349, 352, 390, 396, 464, 549, and 594; 43, 349, 352, 390, 396, 464, 549, 594 and one or more positions selected from 63, 145, 174, 182, 208, 410, 427, 456, 504, and 526; 43, 352, 390, 396, 464, 549, and 594; 43, 349, 352, 390, 396, 464, 549, 594, 410, 526, 415, and 502; 43, 349, 352, 390, 396, 464, 549, 594, 410, 526, 415, 502, and 21; 43, 349, 352, 390, 396, 464, 549, 594, 410, 526, 415, 502, and 67; 43, 349, 352, 390, 396, 464, 549, 594, 410, 526, 415, 502, 21, and 67; 43, 349, 352, 390, 396, 464, 549, 594, 410, 526, 174, 208, 427, 456, and 504; 43, 349, 352, 390, 396, 464, 549, 594, 410, 526, 415, 502, and 139; 43, 349, 352, 390, 396, 464, 549, 594, 410, 526, 415, 502, 339, and 446; 43, 349, 352, 390, 396, 464, 549, 594, 410, 526, 19, 460, 569, and 596; 43, 349, 352, 390, 396, 464, 549, 594, 410, 526, 460, 586, 588, and 608; 43, 349, 352, 390, 396, 464, 549, 594, 410, 526, and 460; 352, 390, 396, 549, 586, and 594; 63, 158, 352, 390, 396, 549, 586, and 594; 164, 165, 352, 363, 390, 396, 410, 549, 586, and 594; 164, 173, 352, 390, 396, 549, 586, and 594; 83, 352, 390, 396, 549, 586, and 594; 8, 43, 174, 349, 352, 390, 396, 427, 464, 549, and 594; or 283, 349, 365, 396, 594, 596, and 131, relative to SEQ ID NO: 5; at least 70% (e.g., having at least 75%, at least 80%, at least 85%, at least 90%, at least 92%, at least 95%, at least 96%, at least 97%, at least 98%, or at least 99%) identity to SEQ ID NO: 6 and amino acid substitutions at positions: 2, 67, 95, and 226; 6 and 316; 38, 95, and 303; 67, 95, and 226; 44 and 76; 44, 76, and 118; 130, 234, 303; 118 and 1201; 118, 1201, and 44; 118, 1201, and 76; 130, 234, and 303; 154 and 269; 221 and 44; 44, 76, 130, 234, and 303; 44, 76, 118, and 1201; 197 and 314; 76, 181, and 194; 76, 118, 252, and 292; 76 and 274; 76, 102, 118, and 307; 12 and 76; 67, 95, and 226; 26 and 76; 22, 76, 319; 154 and 269; 76 and 238; 76, 238, 296, and 328; 7 and 76; 76 and 263; 59, 76, 306, and 316; or 280 and 340, relative to SEQ ID NO: 6; at least 70% (e.g., having at least 75%, at least 80%, at least 85%, at least 90%, at least 92%, at least 95%, at least 96%, at least 97%, at least 98%, or at least 99%) identity to SEQ ID NO: 11 and amino acid substitutions at positions: 105, 109, 131, 148, 279, and 310; or 9, 105, 109, 131, 148, 279, and 310, relative to SEQ ID NO: 11; at least 70% (e.g., having at least 75%, at least 80%, at least 85%, at least 90%, at least 92%, at least 95%, at least 96%, at least 97%, at least 98%, or at least 99%) identity to SEQ ID NO: 12 and amino acid substitutions at positions: 134, 179, 185, 540, 555, 624, and 646; 138, 250, 275, and 421; 303, 405, 520, and 590; 134, 179, 185, 540, 555, and 646, 4 and 49; 4 and 388; 4 and 571; 4, 162, and 480; 4 and 315; 5 and 316; 17 and 156; 38, 108, 497 and 583; 59, 157 and 644; 96, 305, 550 and 642; 106, 160 and 228; 312, 424, 449 and 457; or 376 and 611, relative to SEQ ID NO: 12; at least 70% (e.g., having at least 75%, at least 80%, at least 85%, at least 90%, at least 92%, at least 95%, at least 96%, at least 97%, at least 98%, or at least 99%) identity to SEQ ID NO: 13 and amino acid substitutions at positions: 30, 46, 240, 304, and 316; 30, 46, 240, and 316; 42 and 318; 184, 240, 315, and 345; 211 and 274; 237 and 237; 286 and 350; 317 and 347; 171, 286, and 315; or 328 and 350, relative to SEQ ID NO: 13; or at least 70% (e.g., having at least 75%, at least 80%, at least 85%, at least 90%, at least 92%, at least 95%, at least 96%, at least 97%, at least 98%, or at least 99%) identity to SEQ ID NO: 14 and amino acid substitutions at positions: 82, 110, 115, 164, and 199; 82, 110, 115, 124, 164, and 199; 110, 115, and 164; 110, 115, 164, and 199; 110, 115, 164, 199, and 124; or 110, 115, 164, 199, and 82 or 124, relative to SEQ ID NO: 14.

In some embodiments, the polypeptide comprises an amino acid sequence having: at least 70% identity to SEQ ID NO: 1 and amino acid substitutions at positions: 155; 122 and 155; or 107, 166, and 227, relative to SEQ ID NO: 1; at least 70% identity to SEQ ID NO: 2 and amino acid substitutions at positions: 24, 25, 458, 509, 565, and 600; 22, 347, and 454; or 485, relative to SEQ ID NO: 2; at least 70% identity to SEQ ID NO: 4 and amino acid substitutions at positions: 75 and 182; 88, 147, and 177; 88 and 147; 88, 116 and 147; 88, 147, 170, and 182; 88, 147, 170, 182, and 51 or 180; 88, 147, and 154; 75, 88, and 147; 47, 88, and 147; 88, 128, 147, 170, and 182; or 88, 93, and 147, relative to SEQ ID NO: 4; at least 70% identity to SEQ ID NO: 5 and amino acid substitutions at positions: 352, 390, 396, 594, and 596; 352, 390, 396, 549, and 594; 352, 390, 396, 464, 549, and 594; 289, 352, 390, 396, 549, 594, and 596; 235, 352, 390, 396, 567, and 594; 352, 363, 390, 396, 549, 586, and 594; 352, 390, 396, 549, 580, and 594; 43, 349, 352, 390, 396, 464, 549, 594 and one or more positions selected from 63, 145, 174, 182, 208, 410, 427, 456, 504, and 526; 43, 349, 352, 390, 396, 464, 549, 594, 415 and 502; 43, 349, 352, 390, 396, 464, 549, 594, 415, 502 and 67; 43, 349, 352, 390, 396, 464, 549, 594, 415, 502 and 21; or 43, 349, 352, 390, 396, 464, 549, 594, 415, 502, 21 and 67; relative to SEQ ID NO: 5; or at least 70% identity to SEQ ID NO: 6 and amino acid substitutions at positions: 197, 314, and optionally one of 7, 12, or 114; or 197 and 314; 76, 181, and 194; 76, 118, 252, and 292; 76 and 274; 76, 102, 118, and 307; 12 and 76; 67, 95, and 226; 26 and 76; 22, 76, 319; 154 and 269; 76 and 238; 76, 238, 296, and 328; 7 and 76; 76 and 263; or 59, 76, 306, and 316, relative to SEQ ID NO: 6.

In some embodiments, the polypeptide comprises an amino acid sequence having: at least 70% identity to SEQ ID NO: 1 and amino acid substitutions: M155I; E122A and M155I; or K107M, N166D, and A227P, relative to SEQ ID NO: 1; at least 70% identity to SEQ ID NO: 2 and amino acid substitutions at positions: E24D, L25I, S458N, R509G, H565Y, and I600V; S22P, Y347F, and E454G; or V485F, relative to SEQ ID NO: 2; at least 70% identity to SEQ ID NO: 4 and amino acid substitutions: S75I; F182L; P88T, I147V, and T177I; P88T and I147V; P88T, V116I and I147V; P88T, I147V, V170L, and F182L; P88T, I147V, V170L, F180L, and F182L; G51V, P88T, I147V, V170L, and F182L; P88T, I147V, and F154C; S75I, P88T, and I147V; or P88T, A93T, and I147V, relative to SEQ ID NO: 4; at least 70% identity to SEQ ID NO: 5 and amino acid substitutions: P352T, A390V, D396N, Q594L, and H596Y; P352S, A390V, D396N, Q549R, and Q594L; P352T, A390V, D396N, H464R, Q549R, and Q594L; Q289H, P352T, A390V, D396N, Q549R, Q594L, and H596Y; I235T, P352T, A390V, D396N, K567R, and Q594L; P352T, L363P, A390V, D396N, Q549R, S586A, and Q594L; P352T, A390V, D396N, Q549R, and Q594L; P352T, A390V, D396N, Q549R, T580I, and Q594L; F43S, Y349N or Y349D, P352T, A390V, D396N, H464R, Q549R, Q594L and one or more substitutions selected from R63G, A145S, A174S, I182R, V208M, Q410K, T427S, T456I or T456P, P504S, and V526E; F43S, Y349N or Y349D, P352T, A390V, D396N, H464R, Q549R, Q594L, A415V and T502I; F43S, Y349N or Y349D, P352T, A390V, D396N, H464R, Q549R, Q594L, A415V, T502I, and T21A; F43S, Y349N or Y349D, P352T, A390V, D396N, H464R, Q549R, Q594L, A415V, T502I, and Q67K; or F43S, Y349N or Y349D, P352T, A390V, D396N, H464R, Q549R, Q594L, A415V, T502I, T21A, and Q67K, relative to SEQ ID NO: 5; or at least 70% identity to SEQ ID NO: 6 and amino acid substitutions at positions: R197I, N314K, and optionally one of I7S, L12M, or K114M; R197I and N314K; S76Y, A181S, and V194M; S76Y, K118R, H252R, and K292N; S76Y and I274V; S76Y, A102T, K118R, and V307G; L12M and S76Y; K67N, A95D, and V226E; K26N and S76Y; H22Y, S76Y, and D319N; R154K and E269D; S76Y and A238S; S76Y, A238S, K296N, and V328M; I7V and S76Y; S76Y and S263N; or S59T, S76Y, E306G, and N316D, relative to SEQ ID NO: 6.

In some embodiments, the polypeptide comprises an amino acid sequence having at least 70% identity to SEQ ID NO: 13 and at least one amino acid substitution with a positively charged amino acid. In some embodiments, the positively charged amino acid is arginine or lysine. In select embodiments, the positively charged amino acid is arginine. In some embodiments, the at least one amino acid substitution is at position 2, 5, 6, 7, 8, 9, 10, 12, 38, 39, 40, 41, 42, 43, 44, 45, 46, 47, 48, 64, 65, 66, 67, 68, 69, 70, 222, 224, 225, 227, 228, 229, 231, 232, 233, 234, 235, 255, 256, 257, 258, 277, 286, 287, 337, 338, 339, 340, 345, 346, 347, 348, 349, 350, or a combination thereof. In some embodiments, the at least one amino acid substitution is at positions: 346 and 348; 346, 348 and 349; 346, 348, 349, an 350; 350 and 351; 350, 351, and 352; 350, 351, 352, and 353; 235 and 227; 235 and 345; 235 and 346; 235 and 347; 235 and 348; 235 and 349; 235 and 350; 235, 227, and 349; 5, 235 and 346; 5, 235 and 348; 5, 235 and 349; 227, 235, and 346; or 227, 235, and 348.

In some embodiments, the polypeptide is a fusion polypeptide comprising a first amino acid sequence and a second amino acid sequence. In some embodiments, the fusion polypeptide comprises a first amino acid sequence from one of the disclosed Cas or transposase proteins having at least 70% (e.g., having at least 75%, at least 80%, at least 85%, at least 90%, at least 92%, at least 95%, at least 96%, at least 97%, at least 98%, or at least 99%) identity to any of SEQ ID NOs: 1-14 with one or more amino acid substitutions, deletions, or additions relative to any of SEQ ID NOs: 1-14. In some embodiments, the fusion polypeptide further comprises a second amino acid sequence from one of the disclosed Cas or transposase proteins having at least 70% (e.g., having at least 75%, at least 80%, at least 85%, at least 90%, at least 92%, at least 95%, at least 96%, at least 97%, at least 98%, or at least 99%) identity to any of SEQ ID NOs: 1-14 with one or more amino acid substitutions, deletions, or additions relative to any of SEQ ID NOs: 1-14.

In some embodiments, the fusion polypeptide may comprise two or more of the disclosed transposase proteins (e.g., a first sequence having a sequence encoding a TnsA protein of at least 70% (e.g., having at least 75%, at least 80%, at least 85%, at least 90%, at least 92%, at least 95%, at least 96%, at least 97%, at least 98%, or at least 99%) identity to SEQ ID NO: 1 and a second sequence having a sequence encoding a TnsB protein of at least 70% (e.g., having at least 75%, at least 80%, at least 85%, at least 90%, at least 92%, at least 95%, at least 96%, at least 97%, at least 98%, or at least 99%) identity to SEQ ID NO: 2).

In some embodiments, the first amino acid sequence encodes a TnsA protein and has at least 70% (e.g., having at least 75%, at least 80%, at least 85%, at least 90%, at least 92%, at least 95%, at least 96%, at least 97%, at least 98%, or at least 99%) identity to SEQ ID NO: 1 with one or more amino acid substitutions, deletions, or additions relative to SEQ ID NO: 1 and the second amino acid sequence encodes a TnsB protein and has at least 70% (e.g., having at least 75%, at least 80%, at least 85%, at least 90%, at least 92%, at least 95%, at least 96%, at least 97%, at least 98%, or at least 99%) identity to SEQ ID NO: 2 with one or more amino acid substitutions, deletions, or additions relative to SEQ ID NO: 2.

In some embodiments, the first amino acid sequence comprises one or more amino acid substitutions at positions: 2, 3, 5, 28, 57, 77, 80, 107, 110, 116, 122, 142, 155, 161, 166, 173, 177, 185, 211, 216, 227, and 230, relative to SEQ ID NO: 1. In some embodiments, the second amino acid sequence comprises one or more amino acid substitutions at positions: 2, 5, 22, 24, 25, 29, 75, 141, 199, 215, 319, 347, 364, 370, 383, 439, 454, 458, 485, 509, 533, 538, 565, 581, 586, 595, 596, 597, and 600, relative to SEQ ID NO: 2.

In some embodiments, the first amino acid sequence comprises one or more amino acid substitutions of: A2T, T3I, L5S, T28A, A57T, F77L, Y80D, K107M, K107R, Y110C, Y110D, D116G, E122A, D142E, M155I, K161R, N166D, K173E, Y177N, Y177D, C185R, D211Y, K216E, A227P, G230D, and G230S, relative to SEQ ID NO: 1. In some embodiments, the second amino acid sequence comprises one or more amino acid substitutions of: A2T, A2S, G5R, S22P, E24D, L25I, A29S, P75T, I141T, V199I, S215R, D319V, Y347F, S364N, E370K, N383D, V439A, E454D, E454G, S458N, V485F, R509G, D533A, A538V, H565Y, A581T, H586L, N595K, D596N, D597N, D597Y, and I600V, relative to SEQ ID NO: 2.

In some embodiments, the first amino acid sequence comprises amino acid substitutions at positions: 2 and 230; 107 and 166; 107, 166, and one or both of: 2 and 227; 211 and 110 or 142; 110, 155 and 230; 122 and 155; or 155 and 177, relative to SEQ ID NO: 1. In some embodiments, the second amino acid sequence comprises amino acid substitutions at positions: 2 and 597; 24 and 25; 24, 25, 458, 509, 565, and 600; 75 and 597; 141, 454, 533 and 595; 581, 370, and 454; 370 and 581; 370 and 454; 458 and 509; 458, 509, and 565; 458, 509, 565, and 600; 565, 586, and 596; or 565, 509, 458, 600, and at least one of 24, 25, 29, 215, 319, 364, 383, and 586, relative to SEQ ID NO: 2.

In some embodiments, the first amino acid sequence comprises amino acid substitutions at positions: 107, 166, and 227, relative to SEQ ID NO: 1, and the second amino acid sequence comprises amino acid substitutions at positions: 24, 25, 458, 509, 565, and 600, relative to SEQ ID NO: 2; the first amino acid sequence comprises amino acid substitutions at position 155, relative to SEQ ID NO: 1, and the second amino acid sequence comprises amino acid substitutions at positions: 22, 347, and 454, relative to SEQ ID NO: 2; or the first amino acid sequence comprises amino acid substitutions at positions: 122 and 155, relative to SEQ ID NO: 1, and the second amino acid sequence comprises amino acid substitutions at position: 485, relative to SEQ ID NO: 2.

In some embodiments, the first amino acid sequence comprises amino acid substitutions: K107M, N166D, and A227P, relative to SEQ ID NO: 1, and the second amino acid sequence comprises amino acid substitutions: E24D, L25I, S458N, R509G, H565Y, and I600V, relative to SEQ ID NO: 2; the first amino acid sequence comprises amino acid substitution M155I, relative to SEQ ID NO: 1, and the second amino acid sequence comprises amino acid substitutions S22P, Y347F, and E454G, relative to SEQ ID NO: 2; or the first amino acid sequence comprises amino acid substitutions: E122A and M155I, relative to SEQ ID NO: 1, and the second amino acid sequence comprises amino acid substitution: V485F, relative to SEQ ID NO: 2.

In some embodiments, the first amino acid sequence encodes a TnsA protein and has at least 70% (e.g., having at least 75%, at least 80%, at least 85%, at least 90%, at least 92%, at least 95%, at least 96%, at least 97%, at least 98%, or at least 99%) identity to SEQ ID NO: 4 with one or more amino acid substitutions, deletions, or additions relative to SEQ ID NO: 4. In some embodiments, the second amino acid sequence encodes a TnsA protein and has at least 70% (e.g., having at least 75%, at least 80%, at least 85%, at least 90%, at least 92%, at least 95%, at least 96%, at least 97%, at least 98%, or at least 99%) identity SEQ ID NO: 5 with one or more amino acid substitutions, deletions, or additions relative to SEQ ID NO: 5.

In some embodiments, the first amino acid sequence comprises one or more amino acid substitutions at positions: 4, 5, 9, 10, 12, 21, 23, 25, 26, 31, 32, 34, 35, 37, 41, 45, 47, 48, 51, 52, 55, 60, 61, 65, 67, 69, 72, 75, 79, 80, 82, 87, 88, 90, 91, 93, 94, 96, 98, 99, 100, 103, 106, 108, 113, 116, 125, 126, 128, 129, 135, 139, 143, 146, 147, 149, 153, 154, 156, 158, 159, 160, 162, 164, 166, 167, 168, 169, 170, 177, 179, 180, 182, 183, 185, 187, 188, 190, 191, 192, 193, 195, 196, 200, 204, 207, and 208, relative to SEQ ID NO: 4. In some embodiments, the second amino acid sequence comprises one or more amino acid substitutions at positions: 1, 2, 4, 5, 8, 9, 10, 11, 12, 13, 14, 15, 16, 17, 19, 20, 21, 22, 23, 24, 25, 26, 27, 28, 29, 30, 32, 33, 36, 37, 39, 40, 41, 42, 43, 44, 45, 49, 52, 55, 56, 58, 60, 62, 63, 67, 71, 74, 76, 78, 79, 80, 81, 82, 83, 84, 85, 86, 87, 88, 89, 91, 92, 95, 97, 100, 101, 104, 106, 110, 112, 113, 115, 117, 119, 120, 124, 125, 127, 129, 130, 131, 134, 139, 142, 144, 145, 146, 147, 149, 150, 155, 156, 157, 158, 159, 163, 164, 165, 167, 169, 173, 174, 176, 181, 182, 186, 187, 190, 195, 197, 198, 205, 208, 209, 211, 215, 218, 223, 226, 227, 231, 232, 235, 239, 246, 248, 250, 259, 260, 261, 262, 263, 267, 269, 273, 274, 277, 278, 280, 281, 282, 283, 285, 287, 288, 290, 295, 298, 302, 303, 307, 313, 316, 317, 320, 323, 325, 331, 332, 339, 345, 348, 349, 352, 353, 354, 356, 361, 362, 363, 364, 365, 366, 367, 369, 370, 371, 372, 373, 375, 376, 380, 383, 385, 386, 389, 390, 392, 396, 397, 399, 402, 403, 404, 407, 408, 410, 411, 412, 413, 414, 415, 416, 421, 422, 423, 424, 425, 426, 427, 428, 429, 430, 431, 434, 435, 437, 440, 443, 445, 446, 448, 450, 452, 456, 459, 460, 463, 464, 470, 472, 473, 494, 495, 498, 501, 502, 504, 505, 506, 508, 509, 510, 512, 513, 514, 517, 520, 521, 522, 525, 526, 527, 530, 531, 532, 533, 535, 537, 538, 540, 541, 542, 543, 544, 545, 546, 547, 548, 549, 550, 551, 552, 553, 554, 556, 557, 558, 559, 560, 561, 562, 563, 564, 565, 567, 568, 569, 570, 571, 574, 575, 576, 580, 582, 583, 584, 585, 586, 587, 588, 589, 590, 591, 592, 593, 594, 595, 596, 597, 599, 600, 601, 602, 603, 604, 606, 607, 608, 611, 613, 618, 620, and 656, relative to SEQ ID NO: 5.

In some embodiments, the first amino acid sequence comprises one or more amino acid substitutions of: R4K, N5K, P9S, A10P, N12D, T21I, V23M, S25N, S25R, V26M, V26G, S31N, S32I, E34A, F35L, A37D, H41L, D45N, I47V, E48G, G51V, S52I, E55K, E55D, E60K, F61L, S65T, S65A, P67T, P67L, P67S, P67H, T69A, A72V, A72D, S75I, S75R, S75T, K79E, T80P, K82E, K87R, P88L, P88T, P88A, S90F, K91N, K91E, A93T, A93S, S94N, L96P, R98Q, A99D, A99V, E100K, A103T, A106T, S108A, I113F, V116F, V116I, V125M, V125A, N126T, I128V, I128L, L129P, L135M, S139N, S139G, G143V, G143C, G146D, G146S, I147V, K149E, K149T, K149R, S153I, S153R, S153N, F154C, H156R, H156L, S158N, S158R, G159V, V160A, K162R, N164D, I166L, S167I, S168I, S168R, S168N, Q169R, V170M, V170G, V170L, T177I, T177A, S179R, F180C, F180L, F182C, F182L, G183S, M185I, K187R, G188D, V190I, K191N, A192S, D193N, G195V, G195D, G195S, C196W, T200A, T204I, A207V, A207T, and T208I, relative to SEQ ID NO: 4. In some embodiments, the second amino acid sequence comprises one or more amino acid substitutions of: M1V, M1I, M1L, T2I, T2A, F4L, F5L, F8L, F8V, F8S, D9N, E10K, E10D, S11I, S11R, S11G, L12P, V13M, V13G, V13E, V13L, P14L, L15Q, K16N, K16R, P17T, P17L, P17S, T19I, T19S, T19A, T19P, P20S, P20L, T21A, Q22R, Y23H, V24M, K25R, L26M, D27A, D27G, D28N, D28Y, A29T, A29V, N30K, I32F, I32S, Q33H, L36M, D37A, D37Y, F39L, S40P, D41E, T421, T42K, T42A, F43L, F43S, F43V, K44N, N45D, N45S, Q49R, K52Q, S55A, T56A, D58E, K60Q, S62T, R63K, R63G, Q67R, Q67H, Q67K, D71Y, K74R, E76K, F78C, K79R, G80V, G80D, G81S, G81V, G81D, D82N, V83G, V83M, V83A, V84A, V84G, R85G, R85K, P86L, N87S, R89C, V91G, V91A, A92V, A92T, R95K, K97R, E100D, S101A, D104V, A106D, A106T, D110N, N112H, H113Y, M115R, N117Y, T119A, N120D, N120K, N120S, G124V, D125N, D125E, K127R, F129L, D130N, K131M, E134D, E134G, A139S, A139T, P142S, I144V, A145S, A145T, T146A, A147V, Q149R, Y150H, I155L, V156A, V156L, V156M, K157V, E158A, N159S, V163G, E164A, E164G, E164D, G165D, I167V, I169L, I169T, N173S, N173H, N173T, A174S, A174T, N176D, A181S, I182L, I182V, I182T, A186E, A186T, V187G, V187A, A190T, A190S, F195S, A197P, D198G, D198N, A205S, V208M, P209T, T211I, E215D, E218D, P223S, P223H, L226V, I227V, D231N, E232K, I235V, I235T, R239G, I246V, V248E, V248M, S250I, S259N, Y260C, K261R, S262N, P263L, S267N, A269V, T273I, T273N, H274Y, K277N, K277R, P278S, S280T, L281M, D282E, D282N, A283T, A283S, N285S, E287D, L288M, N290K, F295S, F298I, F298S, V302I, V303M, A307S, N313S, H316R, A317V, S320N, S320R, I323L, I325V, R331K, K332E, I339V, V345L, V345M, E348K, Y349H, Y349D, Y349N, Y349C, P352S, P352T, E353Q, E353D, L354M, G356S, N361D, I362V, I362T, L363P, L363T, L363M, E364G, K365R, E366G, E367G, K369N, K369E, K369M, P370S, E371K, V372M, D373G, I375V, M376I, T380P, T380A, E383K, E383D, F385L, H386Y, I389V, A390V, A390I, V392I, D396N, D396G, D396K, S397P, S399N, S399G, T402I, R403G, R403I, R403K, R403S, I404T, I404V, K407R, K407E, R408K, Q410K, Q410H, Q410R, Q411H, G412V, F413L, D414N, A415V, A415T, Y416C, M421I, N422K, E423K, E423D, E424A, E425K, E426D, T427A, T427S, R428K, F429L, S430A, M431L, R434H, R434C, R434S, I435V, D437G, D437N, T440S, T440I, R443C, G445S, F446L, F446I, Y448C, E450D, E450G, M452I, T456P, T456A, T456I, A459T, D460N, K463N, H464N, H464R, H464S, E470K, V472M, V472A, K473D, K473N, E494D, E494G, S495A, E498A, E498K, C501Y, T502I, T502S, P504S, P504L, T505A, G506Y, G506D, G506L, G506S, T508A, D509E, D509Y, C510Y, S512N, I513L, I513V, I513F, Y514H, K517M, K517N, K517Q, K520R, K521N, I522T, I522V, I522F, E525K, V526E, V526M, I527V, S530N, S530R, K531T, D532G, D532Y, S533Y, G535D, A537T, K538R, K538N, R540K, R540G, M541L, A542T, I543L, H544R, E545A, R546G, R546K, V547M, K548Q, K548R, Q549K, Q549R, E550A, Q551K, E552D, E552K, V553I, F554V, E556K, E556G, S557A, K558R, T559P, T559I, T559A, K560R, A561T, A561G, K562R, K562N, I563L, T564I, A565S, A565V, K567R, K568N, K568R, Q569K, Q569L, Q569R, A570V, Q571R, D574N, V575M, V575A, S576R, T580I, T580A, T582I, T582S, I583V, K584R, V585M, S586P, S586A, S586F, E587A, E588K, E588G, E588D, S589I, S589R, S589N, A590S, A590T, A591V, P592L, V593M, V593A, Q594L, K595R, K595N, H596Y, H596L, H596P, I597T, I597V, N599H, D600L, D600N, D600G, D600V, N601S, N601K, S602A, S602P, S602Y, D603A, D603V, D604G, D604Y, D604N, D606A, D606V, D606Y, D607Y, D607E, D608N, A611T, E613D, R618I, T620P, and A656V, relative to SEQ ID NO: 5.

In some embodiments, the first amino acid sequence comprises amino acid substitutions at positions: 108 and 47 or 208; 170 and 207; 88 and 147; 47, 88 and 147; 88, 147, 170, and 182; 88, 147, 170, 182, and 51 or 180; 88, 147, and 154; 88, 128, 147, 170, and 182; or 170, 207, and 108, relative to SEQ ID NO: 4. In some embodiments, the second amino acid sequence comprises amino acid substitutions at positions: 4, 23 and 590; 19, 169, and 549; 43 and 415; 80 and 593; 80, 144, 593, and 606; 1, 42, 80, 593, and 606; 42, 80, 593, and 606; 156 and 604; 283, 349, and 365; 283, 349, 365, 396, and 594; 283, 349, 365, 396, 594, 596, and 131; 352 and 390; 390, 396, and 594; 396 and 594; 456 and 502; 464 and 502; 464 and 17; 17, 235, 464, and 596; 235, 352, 396, 456, and 606; 415, 456, and 502; 456, 502, and 549; 169, 456, 502, and 549; 80, 456, 502, 593, and 606; 1, 42, 80, 456, 502, 593, and 606; 80, 144, 456, 502, 593, and 606; 19, 169, 456, 502 and 549; 43, 415, 456, and 502; 352, 390, 396, and 594; 352, 390, and 396; 283, 349, 396, and 594; 11, 55, 120, 362, 584, 600, and 604; 43, 84, 144, 349, and 517; 164 and 165; 164 and 173; 362 and 446; 352, 390, 396, 549, and 594; 352, 390, 396, 464, 549, and 594; 43, 349, 352, 390, 396, 464, 549, and 594; 43, 349, 352, 390, 396, 464, 549, 594 and one or more positions selected from 63, 145, 174, 182, 208, 410, 427, 456, 504, and 526; 43, 352, 390, 396, 464, 549, and 594; 43, 349, 352, 390, 396, 464, 549, 594, 410, 526, 415, and 502; 43, 349, 352, 390, 396, 464, 549, 594, 410, 526, 415, 502, and 21; 43, 349, 352, 390, 396, 464, 549, 594, 410, 526, 415, 502, and 67; 43, 349, 352, 390, 396, 464, 549, 594, 410, 526, 415, 502, 21, and 67; 43, 349, 352, 390, 396, 464, 549, 594, 410, 526, 174, 208, 427, 456, and 504; 43, 349, 352, 390, 396, 464, 549, 594, 410, 526, 415, 502, and 139; 43, 349, 352, 390, 396, 464, 549, 594, 410, 526, 415, 502, 339, and 446; 43, 349, 352, 390, 396, 464, 549, 594, 410, 526, 19, 460, 569, and 596; 43, 349, 352, 390, 396, 464, 549, 594, 410, 526, 460, 586, 588, and 608; 43, 349, 352, 390, 396, 464, 549, 594, 410, 526, and 460; 352, 390, 396, 549, 586, and 594; 63, 158, 352, 390, 396, 549, 586, and 594; 164, 165, 352, 363, 390, 396, 410, 549, 586, and 594; 164, 173, 352, 390, 396, 549, 586, and 594; 83, 352, 390, 396, 549, 586, and 594; 8, 43, 174, 349, 352, 390, 396, 427, 464, 549, and 594; or 283, 349, 365, 396, 594, 596, and 131, relative to SEQ ID NO: 5.

In some embodiments, the first amino acid sequence comprises amino acid substitutions at position: 182, relative to SEQ ID NO: 4, and the second amino acid sequence comprises amino acid substitutions at positions: 352, 390, 396, 594, and 596, relative to SEQ ID NO: 5; the first amino acid sequence comprises amino acid substitutions at positions: 88, 147, and 177, relative to SEQ ID NO: 4, and the second amino acid sequence comprises amino acid substitutions at positions: 352, 390, 396, 549, and 594, relative to SEQ ID NO: 5; the first amino acid sequence comprises amino acid substitutions at positions: 88 and 147, relative to SEQ ID NO: 4, and the second amino acid sequence comprises amino acid substitutions at positions: 352, 390, 396, 464, 549, and 594, relative to SEQ ID NO: 5; the first amino acid sequence comprises amino acid substitutions at positions: 88, 116 and 147, relative to SEQ ID NO: 4, and the second amino acid sequence comprises amino acid substitutions at positions: 289, 352, 390, 396, 549, 594, and 596, relative to SEQ ID NO: 5; the first amino acid sequence comprises amino acid substitutions at position: 75, relative to SEQ ID NO: 4, and the second amino acid sequence comprises amino acid substitutions at positions: 235, 352, 390, 396, 567, and 594, relative to SEQ ID NO: 5; the first amino acid sequence comprises amino acid substitutions at positions: 88, 147, 170, and 182, relative to SEQ ID NO: 4, and the second amino acid sequence comprises amino acid substitutions at positions: 352, 363, 390, 396, 549, 586, and 594, relative to SEQ ID NO: 5; the first amino acid sequence comprises amino acid substitutions at positions: 88, 147, 170, 182, and 51 or 180, relative to SEQ ID NO: 4, and the second amino acid sequence comprises amino acid substitutions at positions: 43, 349, 352, 390, 396, 410, 464, 526, 549, and 594, relative to SEQ ID NO: 5; the first amino acid sequence comprises amino acid substitutions at positions: 75, 88, and 147, relative to SEQ ID NO: 4, and the second amino acid sequence comprises amino acid substitutions at positions: 352, 390, 396, 549, and 594, relative to SEQ ID NO: 5; or the first amino acid sequence comprises amino acid substitutions at positions: 88, 93, and 147, relative to SEQ ID NO: 4, and the second amino acid sequence comprises amino acid substitutions at positions: 352, 390, 396, 549, 580, and 594, relative to SEQ ID NO: 5.

In some embodiments, the first amino acid sequence comprises amino acid substitution: F182L, relative to SEQ ID NO: 4, and the second amino acid sequence comprises amino acid substitutions: P352T, A390V, D396N, Q594L, and H596Y, relative to SEQ ID NO: 5; the first amino acid sequence comprises amino acid substitutions: P88T, I147V, and T177I, relative to SEQ ID NO: 4, and the second amino acid sequence comprises amino acid substitutions: P352S, A390V, D396N, Q549R, and Q594L, relative to SEQ ID NO: 5; the first amino acid sequence comprises amino acid substitutions: P88T and I147V, relative to SEQ ID NO: 4, and the second amino acid sequence comprises amino acid substitutions: P352T, A390V, D396N, H464R, Q549R, and Q594L, relative to SEQ ID NO: 5; the first amino acid sequence comprises amino acid substitutions: P88T, V116I and I147V, relative to SEQ ID NO: 4, and the second amino acid sequence comprises amino acid substitutions: Q289H, P352T, A390V, D396N, Q549R, Q594L, and H596Y, relative to SEQ ID NO: 5; the first amino acid sequence comprises amino acid substitution: S75I, relative to SEQ ID NO: 4, and the second amino acid sequence comprises amino acid substitutions: I235T, P352T, A390V, D396N, K567R, and Q594L, relative to SEQ ID NO: 5; the first amino acid sequence comprises amino acid substitutions: P88T, I147V, V170L, and F182L, relative to SEQ ID NO: 4, and the second amino acid sequence comprises amino acid substitutions: P352T, L363P, A390V, D396N, Q549R, S586A, and Q594L, relative to SEQ ID NO: 5; the first amino acid sequence comprises amino acid substitutions at positions: P88T, I147V, V170L, F182L, and G51V or F180L, relative to SEQ ID NO: 4, and the second amino acid sequence comprises amino acid substitutions at positions: F43S, Y349N, P352T, A390V, D396N, Q410K, H464R, V526E, Q549R, and Q594L, relative to SEQ ID NO: 5; the first amino acid sequence comprises amino acid substitutions: S75I, P88T, and I147V, relative to SEQ ID NO: 4, and the second amino acid sequence comprises amino acid substitutions P352T, A390V, D396N, Q549R, and Q594L, relative to SEQ ID NO: 5; or the first amino acid sequence comprises amino acid substitutions: P88T, A93T, and I147V, relative to SEQ ID NO: 4, and the second amino acid sequence comprises amino acid substitutions: P352T, A390V, D396N, Q549R, T580I, and Q594L, relative to SEQ ID NO: 5.

In some embodiments, the polypeptides further comprise one or more peptides fused to the polypeptide. In some embodiments, the one or more peptides comprise a linker peptide fusing the first amino acid sequence to the second amino acid sequence. In some embodiments, the one or more peptides comprise a nuclear localization sequence. In some embodiments, the nuclear localization sequence is a monopartite sequence or a bipartite sequence. In some embodiments, the one or more peptides comprise a tag or detectable label.

Also provided herein are nucleic acids comprising a sequence encoding the disclosed polypeptides and vectors comprising the disclosed nucleic acids.

Further provided are compositions comprising one or more of the disclosed transposon-associated protein or Cas protein polypeptides, or one or more nucleic acids encoding the polypeptides. In some embodiments, the compositions comprise two or more of the disclosed polypeptides, or one or more nucleic acids encoding the polypeptides described herein.

In some embodiments, the composition comprises two or all of a first polypeptide, a second polypeptide, and a third polypeptide (e.g., a first polypeptide having a sequence encoding a TnsA protein of at least 70% (e.g., having at least 75%, at least 80%, at least 85%, at least 90%, at least 92%, at least 95%, at least 96%, at least 97%, at least 98%, or at least 99%) identity to SEQ ID NO: 1 or 4, a second polypeptide having a sequence encoding a TnsB protein of at least 70% (e.g., having at least 75%, at least 80%, at least 85%, at least 90%, at least 92%, at least 95%, at least 96%, at least 97%, at least 98%, or at least 99%) identity to SEQ ID NO: 2 or 5, and/or a third polypeptide having a sequence encoding a TnsC protein of at least 70% (e.g., having at least 75%, at least 80%, at least 85%, at least 90%, at least 92%, at least 95%, at least 96%, at least 97%, at least 98%, or at least 99%) identity to SEQ ID NO: 3 or 6, or alternatively a first polypeptide having a sequence encoding a Cas8 protein of at least 70% (e.g., having at least 75%, at least 80%, at least 85%, at least 90%, at least 92%, at least 95%, at least 96%, at least 97%, at least 98%, or at least 99%) identity to SEQ ID NO: 8 or 12, a second polypeptide having a sequence encoding a Cas7 protein of at least 70% (e.g., having at least 75%, at least 80%, at least 85%, at least 90%, at least 92%, at least 95%, at least 96%, at least 97%, at least 98%, or at least 99%) identity to SEQ ID NO: 9 or 13, and/or a third polypeptide having a sequence encoding a Cas6 protein of at least 70% (e.g., having at least 75%, at least 80%, at least 85%, at least 90%, at least 92%, at least 95%, at least 96%, at least 97%, at least 98%, or at least 99%) identity to SEQ ID NO: 10 or 14).

In some embodiments, the first polypeptide comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 1 with one or more amino acid substitutions, deletions, or additions relative to SEQ ID NO: 1. In some embodiments, the second polypeptide comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 2 with one or more amino acid substitutions, deletions, or additions relative to SEQ ID NO: 2. In some embodiments, the third polypeptide comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 3 with one or more amino acid substitutions, deletions, or additions relative to SEQ ID NO: 3.

In some embodiments, the first polypeptide comprises one or more amino acid substitutions at positions: 2, 3, 5, 28, 57, 77, 80, 107, 110, 116, 122, 142, 155, 161, 166, 173, 177, 185, 211, 216, 227, and 230, relative to SEQ ID NO: 1. In some embodiments, the second polypeptide comprises one or more amino acid substitutions at positions: 2, 5, 22, 24, 25, 29, 75, 141, 199, 215, 319, 347, 364, 370, 383, 439, 454, 458, 485, 509, 533, 538, 565, 581, 586, 595, 596, 597, and 600, relative to SEQ ID NO: 2. In some embodiments, the third polypeptide comprises one or more amino acid substitutions at positions: 9, 15, 16, 18, 21, 64, 81, 86, 87, 99, 109, 142, 147, 153, 168, 180, 216, 230, 285, and 304, relative to SEQ ID NO: 3.

In some embodiments, the first polypeptide comprises one or more amino acid substitutions of: A2T, T3I, L5S, T28A, A57T, F77L, Y80D, K107M, K107R, Y110C, Y110D, D116G, E122A, D142E, M155I, K161R, N166D, K173E, Y177N, Y177D, C185R, D211Y, K216E, A227P, G230D and G230S, relative to SEQ ID NO: 1. In some embodiments, the second polypeptide comprises one or more amino acid substitutions of: A2T, A2S, G5R, S22P, E24D, L25I, A29S, P75T, I141T, V199I, S215R, D319V, Y347F, S364N, E370K, N383D, V439A, E454D, E454G, S458N, V485F, R509G, D533A, A538V, H565Y, A581T, H586L, N595K, D596N, D597N, D597Y, and I600V, relative to SEQ ID NO: 2. In some embodiments, the third polypeptide comprises one or more amino acid substitutions of: 19V, A15V, F16Y, S18F, S21N, N64D, H81Y, D86Y, N87K, V99I, E109D, E142K, V147I, N153D, I168M, A180E, A216S, L230F, K285E, and R304R, relative to SEQ ID NO: 3.

In some embodiments, the first polypeptide comprises amino acid substitutions at positions: 2 and 230; 107 and 166; 107, 166, and one or both of: 2 and 227; 211 and 110 or 142; 110, 155 and 230; 122 and 155; or 155 and 177, relative to SEQ ID NO: 1. In some embodiments, second polypeptide comprises amino acid substitutions at positions: 2 and 597; 24 and 25; 24, 25, 458, 509, 565, and 600; 75 and 597; 141, 454, 533 and 595; 581, 370, and 454; 370 and 581; 370 and 454; 458 and 509; 458, 509 and 565; 458, 509, 565, and 600; 565, 586, and 596; or 565, 509, 458, 600 and at least one of 24, 25, 29, 215, 319, 364, 383, and 586, relative to SEQ ID NO: 2. In some embodiments, the third polypeptide comprises amino acid substitutions at positions: 142 and 216, relative to SEQ ID NO: 3.

In some embodiments, the first polypeptide comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 4 with one or more amino acid substitutions, deletions, or additions relative to SEQ ID NO: 4. In some embodiments, the second polypeptide comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 5 with one or more amino acid substitutions, deletions, or additions relative to SEQ ID NO: 5. In some embodiments, the third polypeptide comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 6 with one or more amino acid substitutions, deletions, or additions relative to SEQ ID NO: 6.

In some embodiments, the first polypeptide comprises one or more amino acid substitutions at positions: 4, 5, 9, 10, 12, 21, 23, 25, 26, 31, 32, 34, 35, 37, 41, 45, 47, 48, 51, 52, 55, 60, 61, 65, 67, 69, 72, 75, 79, 80, 82, 87, 88, 90, 91, 93, 94, 96, 98, 99, 100, 103, 106, 108, 113, 116, 125, 126, 128, 129, 135, 139, 143, 146, 147, 149, 153, 154, 156, 158, 159, 160, 162, 164, 166, 167, 168, 169, 170, 177, 179, 180, 182, 183, 185, 187, 188, 190, 191, 192, 193, 195, 196, 200, 204, 207, and 208, relative to SEQ ID NO: 4. In some embodiments, the second polypeptide comprises one or more amino acid substitutions at positions: 1, 2, 4, 5, 8, 9, 10, 11, 12, 13, 14, 15, 16, 17, 19, 20, 21, 22, 23, 24, 25, 26, 27, 28, 29, 30, 32, 33, 36, 37, 39, 40, 41, 42, 43, 44, 45, 49, 52, 55, 56, 58, 60, 62, 63, 67, 71, 74, 76, 78, 79, 80, 81, 82, 83, 84, 85, 86, 87, 88, 89, 91, 92, 95, 97, 100, 101, 104, 106, 110, 112, 113, 115, 117, 119, 120, 124, 125, 127, 129, 130, 131, 134, 139, 142, 144, 145, 146, 147, 149, 150, 155, 156, 157, 158, 159, 163, 164, 165, 167, 169, 173, 174, 176, 181, 182, 186, 187, 190, 195, 197, 198, 205, 208, 209, 211, 215, 218, 223, 226, 227, 231, 232, 235, 239, 246, 248, 250, 259, 260, 261, 262, 263, 267, 269, 273, 274, 277, 278, 280, 281, 282, 283, 285, 287, 288, 290, 295, 298, 302, 303, 307, 313, 316, 317, 320, 323, 325, 331, 332, 339, 345, 348, 349, 352, 353, 354, 356, 361, 362, 363, 364, 365, 366, 367, 369, 370, 371, 372, 373, 375, 376, 380, 383, 385, 386, 389, 390, 392, 396, 397, 399, 402, 403, 404, 407, 408, 410, 411, 412, 413, 414, 415, 416, 421, 422, 423, 424, 425, 426, 427, 428, 429, 430, 431, 434, 435, 437, 440, 443, 445, 446, 448, 450, 452, 456, 459, 460, 463, 464, 470, 472, 473, 494, 495, 498, 501, 502, 504, 505, 506, 508, 509, 510, 512, 513, 514, 517, 520, 521, 522, 525, 526, 527, 530, 531, 532, 533, 535, 537, 538, 540, 541, 542, 543, 544, 545, 546, 547, 548, 549, 550, 551, 552, 553, 554, 556, 557, 558, 559, 560, 561, 562, 563, 564, 565, 567, 568, 569, 570, 571, 574, 575, 576, 580, 582, 583, 584, 585, 586, 587, 588, 589, 590, 591, 592, 593, 594, 595, 596, 597, 599, 600, 601, 602, 603, 604, 606, 607, 608, 611, 613, 618, 620, and 656, relative to SEQ ID NO: 5. In some embodiments, the third polypeptide comprises one or more amino acid substitutions at positions: 1, 2, 3, 5, 6, 7, 9, 11, 12, 14, 21, 22, 26, 27, 31, 35, 38, 43, 44, 46, 47, 54, 59, 60, 61, 64, 65, 67, 68, 71, 72, 74, 76, 79, 80, 81, 84, 89, 95, 102, 105, 109, 110, 111, 112, 113, 114, 116, 118, 119, 120, 123, 129, 130, 131, 132, 134, 142, 145, 146, 147, 148, 150, 154, 155, 166, 169, 178, 180, 181, 183, 184, 187, 190, 194, 197, 201, 204, 207, 209, 213, 219, 221, 225, 226, 227, 229, 232, 233, 234, 236, 238, 241, 246, 251, 252, 256, 257, 261, 263, 265, 267, 269, 271, 272, 274, 280, 281, 285, 286, 288, 291, 292, 296, 299, 301, 303, 304, 306, 307, 308, 310, 313, 314, 316, 317, 318, 319, 320, 323, 324, 326, 328, 330, 331, 332, 340, 341, 343, 344, 355, 412, 418, 427, 514, 1198, 1201, 1206, 1212, 1260, and 1282, relative to SEQ ID NO: 6.

In some embodiments, the first polypeptide comprises one or more amino acid substitutions of: R4K, N5K, P9S, A10P, N12D, T21I, V23M, S25N, S25R, V26M, V26G, S31N, S32I, E34A, F35L, A37D, H41L, D45N, I47V, E48G, G51V, S52I, E55K, E55D, E60K, F61L, S65T, S65A, P67T, P67L, P67S, P67H, T69A, A72V, A72D, S75I, S75R, S75T, K79E, T80P, K82E, K87R, P88L, P88T, P88A, S90F, K91N, K91E, A93T, A93S, S94N, L96P, R98Q, A99D, A99V, E100K, A103T, A106T, S108A, I113F, V116F, V116I, V125M, V125A, N126T, I128V, I128L, L129P, L135M, S139N, S139G, G143V, G143C, G146D, G146S, I147V, K149E, K149T, K149R, S153I, S153R, S153N, F154C, H156R, H156L, S158N, S158R, G159V, V160A, K162R, N164D, I166L, S167I, S168I, S168R, S168N, Q169R, V170M, V170G, V170L, T177I, T177A, S179R, F180C, F180L, F182C, F182L, G183S, M185I, K187R, G188D, V190I, K191N, A192S, D193N, G195V, G195D, G195S, C196W, T200A, T204I, A207V, A207T, and T208I, relative to SEQ ID NO: 4. In some embodiments, the second polypeptide comprises one or more amino acid substitutions of: M1V, M1I, M1L, T2I, T2A, F4L, F5L, F8L, F8V, F8S, D9N, E10K, E10D, S11I, S11R, S11G, L12P, V13M, V13G, V13E, V13L, P14L, L15Q, K16N, K16R, P17T, P17L, P17S, T19I, T19S, T19A, T19P, P20S, P20L, T21A, Q22R, Y23H, V24M, K25R, L26M, D27A, D27G, D28N, D28Y, A29T, A29V, N30K, I32F, I32S, Q33H, L36M, D37A, D37Y, F39L, S40P, D41E, T42I, T42K, T42A, F43L, F43S, F43V, K44N, N45D, N45S, Q49R, K52Q, S55A, T56A, D58E, K60Q, S62T, R63K, R63G, Q67R, Q67H, Q67K, D71Y, K74R, E76K, F78C, K79R, G80V, G80D, G81S, G81V, G81D, D82N, V83G, V83M, V83A, V84A, V84G, R85G, R85K, P86L, N87S, R89C, V91G, V91A, A92V, A92T, R95K, K97R, E100D, S101A, D104V, A106D, A106T, D110N, N112H, H113Y, M115R, N117Y, T119A, N120D, N120K, N120S, G124V, D125N, D125E, K127R, F129L, D130N, K131M, E134D, E134G, A139S, A139T, P142S, I144V, A145S, A145T, T146A, A147V, Q149R, Y150H, I155L, V156A, V156L, V156M, K157V, E158A, N159S, V163G, E164A, E164G, E164D, G165D, I167V, I169L, I169T, N173S, N173H, N173T, A174S, A174T, N176D, A181S, I182L, I182V, I182T, A186E, A186T, V187G, V187A, A190T, A190S, F195S, A197P, D198G, D198N, A205S, V208M, P209T, T211I, E215D, E218D, P223S, P223H, L226V, I227V, D231N, E232K, I235V, I235T, R239G, I246V, V248E, V248M, S250I, S259N, Y260C, K261R, S262N, P263L, S267N, A269V, T273I, T273N, H274Y, K277N, K277R, P278S, S280T, L281M, D282E, D282N, A283T, A283S, N285S, E287D, L288M, N290K, F295S, F298I, F298S, V302I, V303M, A307S, N313S, H316R, A317V, S320N, S320R, I323L, I325V, R331K, K332E, I339V, V345L, V345M, E348K, Y349H, Y349D, Y349N, Y349C, P352S, P352T, E353Q, E353D, L354M, G356S, N361D, I362V, I362T, L363P, L363T, L363M, E364G, K365R, E366G, E367G, K369N, K369E, K369M, P370S, E371K, V372M, D373G, I375V, M376I, T380P, T380A, E383K, E383D, F385L, H386Y, I389V, A390V, A390I, V392I, D396N, D396G, D396K, S397P, S399N, S399G, T402I, R403G, R403I, R403K, R403S, I404T, I404V, K407R, K407E, R408K, Q410K, Q410H, Q410R, Q411H, G412V, F413L, D414N, A415V, A415T, Y416C, M421I, N422K, E423K, E423D, E424A, E425K, E426D, T427A, T427S, R428K, F429L, S430A, M431L, R434H, R434C, R434S, I435V, D437G, D437N, T440S, T440I, R443C, G445S, F446L, F446I, Y448C, E450D, E450G, M452I, T456P, T456A, T456I, A459T, D460N, K463N, H464N, H464R, H464S, E470K, V472M, V472A, K473D, K473N, E494D, E494G, S495A, E498A, E498K, C501Y, T502I, T502S, P504S, P504L, T505A, G506Y, G506D, G506L, G506S, T508A, D509E, D509Y, C510Y, S512N, I513L, I513V, I513F, Y514H, K517M, K517N, K517Q, K520R, K521N, I522T, I522V, I522F, E525K, V526E, V526M, I527V, S530N, S530R, K531T, D532G, D532Y, S533Y, G535D, A537T, K538R, K538N, R540K, R540G, M541L, A542T, I543L, H544R, E545A, R546G, R546K, V547M, K548Q, K548R, Q549K, Q549R, E550A, Q551K, E552D, E552K, V553I, F554V, E556K, E556G, S557A, K558R, T559P, T559I, T559A, K560R, A561T, A561G, K562R, K562N, I563L, T564I, A565S, A565V, K567R, K568N, K568R, Q569K, Q569L, Q569R, A570V, Q571R, D574N, V575M, V575A, S576R, T580I, T580A, T582I, T582S, I583V, K584R, V585M, S586P, S586A, S586F, E587A, E588K, E588G, E588D, S589I, S589R, S589N, A590S, A590T, A591V, P592L, V593M, V593A, Q594L, K595R, K595N, H596Y, H596L, H596P, I597T, I597V, N599H, D600L, D600N, D600G, D600V, N601S, N601K, S602A, S602P, S602Y, D603A, D603V, D604G, D604Y, D604N, D606A, D606V, D606Y, D607Y, D607E, D608N, A611T, E613D, R618I, T620P, and A656V, relative to SEQ ID NO: 5. In some embodiments, the third polypeptide comprises one or more amino acid substitutions of: M1L, M1V, N2S, A3T, T5P, T5A, T5S, E6D, 17S, 17V, 19F, Q11R, L12M, N14D, N14S, M21I, H22P, H22Y, K26N, K26R, T27I, M31I, L35R, N38S, S43P, D44N, D44G, Q46L, C47S, T54I, S59T, H60Y, T61A, H64Y, Y65H, K67N, K67R, R68Q, A71G, T72A, N74D, S76C, S76Y, T79I, M80I, P81S, V84L, R89L, A95D, A95T, A102T, E105D, E105K, S109N, S109R, S110P, Q111R, I112T, K113N, K113E, K114N, K114M, K114E, G116D, K118N, K118R, T119I, D120V, K123N, L129M, I130V, K131R, A132S, K134M, K134N, F142V, L145M, I146T, E147K, F148S, S150F, R154K, Q155H, E166D, K169E, P178S, A180V, A181T, A181S, I183V, A184S, A184T, A184V, P187S, A190T, A190V, V194M, V194A, R197I, Y201N, L204M, D207N, K209N, Q213H, Q213V, A219S, K221N, D225N, V226E, P227T, K229E, S232N, K233N, K233R, N234H, T236A, A238V, A238S, A241S, E246D, K251N, H252Y, H252R, E256D, A257S, A261V, S263I, S263N, N265D, Y267C, E269K, E269D, K271E, K271R, H272Y, I274V, F280L, D281N, D281G, K285G, K286N, K288R, S291F, S291P, K292N, K296R, K296N, I299S, D301G, E303D, I304T, I304V, E306G, V307L, V307G, V307A, V307D, V307G, I308N, N310S, Y313H, N314K, N316K, N316D, A317D, L318Q, D319N, P320S, P320L, M323I, L324M, D326N, V328M, V328A, A330D, I331V, V332G, S340L, T341A, A343G, S344N, I355V, F412V, V418F, Y427C, R514K, S1198L, A1201V, G1206S, C1212G, F1260L, and V1282M, relative to SEQ ID NO: 6.

In some embodiments, the first polypeptide comprises amino acid substitutions at positions: 108 and 47 or 208; 170 and 207; 88 and 147; 47, 88 and 147; 88, 147, 170, and 182; 88, 147, 170, 182, and 51 or 180; 88, 147, and 154; 88, 128, 147, 170, and 182; or 170, 207, and 108, relative to SEQ ID NO: 4. In some embodiments, the second polypeptide comprises amino acid substitutions at positions: 4, 23 and 590; 19, 169, and 549; 43 and 415; 80 and 593; 80, 144, 593, and 606; 1, 42, 80, 593, and 606; 42, 80, 593, and 606; 156 and 604; 283, 349, and 365; 283, 349, 365, 396, and 594; 283, 349, 365, 396, 594, 596, and 131; 352 and 390; 390, 396, and 594; 396 and 594; 456 and 502; 464 and 502; 464 and 17; 17, 235, 464, and 596; 235, 352, 396, 456, and 606; 415, 456, and 502; 456, 502, and 549; 169, 456, 502, and 549; 80, 456, 502, 593, and 606; 1, 42, 80, 456, 502, 593, and 606; 80, 144, 456, 502, 593, and 606; 19, 169, 456, 502 and 549; 43, 415, 456, and 502; 352, 390, 396, and 594; 352, 390, and 396; 283, 349, 396, and 594; 11, 55, 120, 362, 584, 600, and 604; 43, 84, 144, 349, and 517; 164 and 165; 164 and 173; 362 and 446; 352, 390, 396, 549, and 594; 352, 390, 396, 464, 549, and 594; 43, 349, 352, 390, 396, 464, 549, and 594; 43, 349, 352, 390, 396, 464, 549, 594 and one or more positions selected from 63, 145, 174, 182, 208, 410, 427, 456, 504, and 526; 43, 352, 390, 396, 464, 549, and 594; 43, 349, 352, 390, 396, 464, 549, 594, 410, 526, 415, and 502; 43, 349, 352, 390, 396, 464, 549, 594, 410, 526, 415, 502, and 21; 43, 349, 352, 390, 396, 464, 549, 594, 410, 526, 415, 502, and 67; 43, 349, 352, 390, 396, 464, 549, 594, 410, 526, 415, 502, 21, and 67; 43, 349, 352, 390, 396, 464, 549, 594, 410, 526, 174, 208, 427, 456, and 504; 43, 349, 352, 390, 396, 464, 549, 594, 410, 526, 415, 502, and 139; 43, 349, 352, 390, 396, 464, 549, 594, 410, 526, 415, 502, 339, and 446; 43, 349, 352, 390, 396, 464, 549, 594, 410, 526, 19, 460, 569, and 596; 43, 349, 352, 390, 396, 464, 549, 594, 410, 526, 460, 586, 588, and 608; 43, 349, 352, 390, 396, 464, 549, 594, 410, 526, and 460; 352, 390, 396, 549, 586, and 594; 63, 158, 352, 390, 396, 549, 586, and 594; 164, 165, 352, 363, 390, 396, 410, 549, 586, and 594; 164, 173, 352, 390, 396, 549, 586, and 594; 83, 352, 390, 396, 549, 586, and 594; 8, 43, 174, 349, 352, 390, 396, 427, 464, 549, and 594; or 283, 349, 365, 396, 594, 596, and 131, relative to SEQ ID NO: 5. In some embodiments, the third polypeptide comprises amino acid substitutions at positions: 2, 67, 95, and 226; 6 and 316; 38, 95, 303; 67, 95, and 226; 44 and 76; 44, 76, and 118; 130, 234, 303; 118 and 1201; 118, 1201, and 44; 118, 1201, and 76; 130, 234, and 303; 154 and 269; 221 and 44; 44, 76, 130, 234, and 303; 44, 76, 118, and 1201; 197 and 314; 76, 181, and 194; 76, 118, 252, and 292; 76 and 274; 76, 102, 118, and 307; 12 and 76; 67, 95, and 226; 26 and 76; 22, 76, 319; 154 and 269; 76 and 238; 76, 238, 296, and 328; 7 and 76; 76 and 263; 59, 76, 306, and 316; or 280 and 340, relative to SEQ ID NO: 6.

In some embodiments, the first polypeptide comprises amino acid substitutions at positions: 88 and 147, relative to SEQ ID NO: 4, the second polypeptide comprises amino acid substitutions at positions: 352, 390, 396, 464, 549, and 594, relative to SEQ ID NO: 5, and the third polypeptide comprises amino acid substitutions at positions: 197, 314, and optionally one of 7, 12, or 114, relative to SEQ ID NO: 6; the first polypeptide comprises amino acid substitutions at positions: 88 and 147, relative to SEQ ID NO: 4, the second polypeptide comprises amino acid substitutions at positions: 352, 390, 396, 464, 549, and 594, relative to SEQ ID NO: 5, and the third polypeptide comprises amino acid substitutions at positions: 76, 181, and 194, relative to SEQ ID NO: 6; the first polypeptide comprises amino acid substitutions at positions: 88, 147, 170, and 182, relative to SEQ ID NO: 4, the second polypeptide comprises amino acid substitutions at positions: 352, 363, 390, 396, 549, 586, and 594, relative to SEQ ID NO: 5, and the third polypeptide comprises amino acid substitutions at positions: 197, 314, and optionally one of 7, 12, or 114, relative to SEQ ID NO: 6; the first polypeptide comprises amino acid substitutions at positions: 88, 147, 170, 180, and 182, relative to SEQ ID NO: 4, the second polypeptide comprises amino acid substitutions at positions: 43, 349, 352, 390, 396, 410, 464, 526, 549, and 594, relative to SEQ ID NO: 5, and the third polypeptide comprises amino acid substitutions at positions: 197 and 314, relative to SEQ ID NO: 6; or the first polypeptide comprises amino acid substitutions at positions: 88, 147, 170, and 182, relative to SEQ ID NO: 4, the second polypeptide comprises amino acid substitutions at positions: 352, 363, 390, 396, 549, 586, and 594, relative to SEQ ID NO: 5, and the third polypeptide comprises amino acid substitutions at positions: 76, 181, and 194, relative to SEQ ID NO: 6.

In some embodiments, the first polypeptide comprises amino acid substitutions: P88T and I147V, relative to SEQ ID NO: 4, the second polypeptide comprises amino acid substitutions: P352T, A390V, D396N, H464R, Q549R, and Q594L, relative to SEQ ID NO: 5, and the third polypeptide comprises amino acid substitutions: R197I, N314K, and optionally one of I7S, L12M, or K114M, relative to SEQ ID NO: 6; the first polypeptide comprises amino acid substitutions: P88T and I147V, relative to SEQ ID NO: 4, the second polypeptide comprises amino acid substitutions: P352T, A390V, D396N, H464R, Q549R, and Q594L, relative to SEQ ID NO: 5, and the third polypeptide comprises amino acid substitutions: S76Y, A181S, and V194M, relative to SEQ ID NO: 6; the first polypeptide comprises amino acid substitutions: 88, 147, 170, and 182, relative to SEQ ID NO: 4, the second polypeptide comprises amino acid substitutions: P352T, L363P, A390V, D396N, Q549R, S586A, and Q594L, relative to SEQ ID NO: 5, and the third polypeptide comprises amino acid substitutions: R197I, N314K, and optionally one of I7S, L12M, or K114M, relative to SEQ ID NO: 6; the first polypeptide comprises amino acid substitutions at positions: P88T, I147V, V170L, F180L, and F182L, relative to SEQ ID NO: 4, the second polypeptide comprises amino acid substitutions at positions: F43S, Y349N, P352T, A390V, D396N, Q410K, H464R, V526E, Q549R, and Q594L, relative to SEQ ID NO: 5, and the third polypeptide comprises amino acid substitutions at positions: R197I and N314K, relative to SEQ ID NO: 6; or the first polypeptide comprises amino acid substitutions: 88, 147, 170, and 182, relative to SEQ ID NO: 4, the second polypeptide comprises amino acid substitutions: P352T, L363P, A390V, D396N, Q549R, S586A, and Q594L, relative to SEQ ID NO: 5, and the third polypeptide comprises amino acid substitutions: S76Y, A181S, and V194M, relative to SEQ ID NO: 6.

In some embodiments, the first polypeptide comprises an amino acid sequence of SEQ ID NO: 4; the second polypeptide comprises an amino acid sequence having at least 70% identity to SEQ ID NO: 5 with one or more amino acid substitutions, deletions, or additions relative to SEQ ID NO: 5; and the third polypeptide comprises an amino acid sequence having at least 70% identity to SEQ ID NO: 6 with one or more amino acid substitutions, deletions, or additions relative to SEQ ID NO: 6. In some embodiments, the second polypeptide comprises one or more amino acid substitutions at positions: 1, 2, 4, 5, 8, 9, 10, 11, 12, 13, 14, 15, 16, 17, 19, 20, 21, 22, 23, 24, 25, 26, 27, 28, 29, 30, 32, 33, 36, 37, 39, 40, 41, 42, 43, 44, 45, 49, 52, 55, 56, 58, 60, 62, 63, 67, 71, 74, 76, 78, 79, 80, 81, 82, 83, 84, 85, 86, 87, 88, 89, 91, 92, 95, 97, 100, 101, 104, 106, 110, 112, 113, 115, 117, 119, 120, 124, 125, 127, 129, 130, 131, 134, 139, 142, 144, 145, 146, 147, 149, 150, 155, 156, 157, 158, 159, 163, 164, 165, 167, 169, 173, 174, 176, 181, 182, 186, 187, 190, 195, 197, 198, 205, 208, 209, 211, 215, 218, 223, 226, 227, 231, 232, 235, 239, 246, 248, 250, 259, 260, 261, 262, 263, 267, 269, 273, 274, 277, 278, 280, 281, 282, 283, 285, 287, 288, 290, 295, 298, 302, 303, 307, 313, 316, 317, 320, 323, 325, 331, 332, 339, 345, 348, 349, 352, 353, 354, 356, 361, 362, 363, 364, 365, 366, 367, 369, 370, 371, 372, 373, 375, 376, 380, 383, 385, 386, 389, 390, 392, 396, 397, 399, 402, 403, 404, 407, 408, 410, 411, 412, 413, 414, 415, 416, 421, 422, 423, 424, 425, 426, 427, 428, 429, 430, 431, 434, 435, 437, 440, 443, 445, 446, 448, 450, 452, 456, 459, 460, 463, 464, 470, 472, 473, 494, 495, 498, 501, 502, 504, 505, 506, 508, 509, 510, 512, 513, 514, 517, 520, 521, 522, 525, 526, 527, 530, 531, 532, 533, 535, 537, 538, 540, 541, 542, 543, 544, 545, 546, 547, 548, 549, 550, 551, 552, 553, 554, 556, 557, 558, 559, 560, 561, 562, 563, 564, 565, 567, 568, 569, 570, 571, 574, 575, 576, 580, 582, 583, 584, 585, 586, 587, 588, 589, 590, 591, 592, 593, 594, 595, 596, 597, 599, 600, 601, 602, 603, 604, 606, 607, 608, 611, 613, 618, 620, and 656, relative to SEQ ID NO: 5; and/or the third polypeptide comprises one or more amino acid substitutions at positions: 1, 2, 3, 5, 6, 7, 9, 11, 12, 14, 21, 22, 26, 27, 31, 35, 38, 43, 44, 46, 47, 54, 59, 60, 61, 64, 65, 67, 68, 71, 72, 74, 76, 79, 80, 81, 84, 89, 95, 102, 105, 109, 110, 111, 112, 113, 114, 116, 118, 119, 120, 123, 129, 130, 131, 132, 134, 142, 145, 146, 147, 148, 150, 154, 155, 166, 169, 178, 180, 181, 183, 184, 187, 190, 194, 197, 201, 204, 207, 209, 213, 219, 221, 225, 226, 227, 229, 232, 233, 234, 236, 238, 241, 246, 251, 252, 256, 257, 261, 263, 265, 267, 269, 271, 272, 274, 280, 281, 285, 286, 288, 291, 292, 296, 299, 301, 303, 304, 306, 307, 308, 310, 313, 314, 316, 317, 318, 319, 320, 323, 324, 326, 328, 330, 331, 332, 340, 341, 343, 344, 355, 412, 418, 427, 514, 1198, 1201, 1206, 1212, 1260, and 1282, relative to SEQ ID NO: 6.

In some embodiments, the second polypeptide comprises one or more amino acid substitutions of: M1V, M1I, M1L, T2I, T2A, F4L, F5L, F8L, F8V, F8S, D9N, E10K, E10D, S11I, S11R, S11G, L12P, V13M, V13G, V13E, V13L, P14L, L15Q, K16N, K16R, P17T, P17L, P17S, T19I, T19S, T19A, T19P, P20S, P20L, T21A, Q22R, Y23H, V24M, K25R, L26M, D27A, D27G, D28N, D28Y, A29T, A29V, N30K, I32F, I32S, Q33H, L36M, D37A, D37Y, F39L, S40P, D41E, T42I, T42K, T42A, F43L, F43S, F43V, K44N, N45D, N45S, Q49R, K52Q, S55A, T56A, D58E, K60Q, S62T, R63K, R63G, Q67R, Q67H, Q67K, D71Y, K74R, E76K, F78C, K79R, G80V, G80D, G81S, G81V, G81D, D82N, V83G, V83M, V83A, V84A, V84G, R85G, R85K, P86L, N87S, R89C, V91G, V91A, A92V, A92T, R95K, K97R, E100D, S101A, D104V, A106D, A106T, D110N, N112H, H113Y, M115R, N117Y, T119A, N120D, N120K, N120S, G124V, D125N, D125E, K127R, F129L, D130N, K131M, E134D, E134G, A139S, A139T, P142S, I144V, A145S, A145T, T146A, A147V, Q149R, Y150H, I155L, V156A, V156L, V156M, K157V, E158A, N159S, V163G, E164A, E164G, E164D, G165D, I167V, I169L, I169T, N173S, N173H, N173T, A174S, A174T, N176D, A181S, I182L, I182V, I182T, A186E, A186T, V187G, V187A, A190T, A190S, F195S, A197P, D198G, D198N, A205S, V208M, P209T, T211I, E215D, E218D, P223S, P223H, L226V, I227V, D231N, E232K, I235V, I235T, R239G, I246V, V248E, V248M, S250I, S259N, Y260C, K261R, S262N, P263L, S267N, A269V, T273I, T273N, H274Y, K277N, K277R, P278S, S280T, L281M, D282E, D282N, A283T, A283S, N285S, E287D, L288M, N290K, F295S, F298I, F298S, V302I, V303M, A307S, N313S, H316R, A317V, S320N, S320R, I323L, I325V, R331K, K332E, I339V, V345L, V345M, E348K, Y349H, Y349D, Y349N, Y349C, P352S, P352T, E353Q, E353D, L354M, G356S, N361D, I362V, I362T, L363P, L363T, L363M, E364G, K365R, E366G, E367G, K369N, K369E, K369M, P370S, E371K, V372M, D373G, I375V, M376I, T380P, T380A, E383K, E383D, F385L, H386Y, I389V, A390V, A390I, V392I, D396N, D396G, D396K, S397P, S399N, S399G, T402I, R403G, R403I, R403K, R403S, I404T, I404V, K407R, K407E, R408K, Q410K, Q410H, Q410R, Q411H, G412V, F413L, D414N, A415V, A415T, Y416C, M421I, N422K, E423K, E423D, E424A, E425K, E426D, T427A, T427S, R428K, F429L, S430A, M431L, R434H, R434C, R434S, I435V, D437G, D437N, T440S, T440I, R443C, G445S, F446L, F446I, Y448C, E450D, E450G, M452I, T456P, T456A, T456I, A459T, D460N, K463N, H464N, H464R, H464S, E470K, V472M, V472A, K473D, K473N, E494D, E494G, S495A, E498A, E498K, C501Y, T502I, T502S, P504S, P504L, T505A, G506Y, G506D, G506L, G506S, T508A, D509E, D509Y, C510Y, S512N, I513L, I513V, I513F, Y514H, K517M, K517N, K517Q, K520R, K521N, I522T, I522V, I522F, E525K, V526E, V526M, I527V, S530N, S530R, K531T, D532G, D532Y, S533Y, G535D, A537T, K538R, K538N, R540K, R540G, M541L, A542T, I543L, H544R, E545A, R546G, R546K, V547M, K548Q, K548R, Q549K, Q549R, E550A, Q551K, E552D, E552K, V553I, F554V, E556K, E556G, S557A, K558R, T559P, T559I, T559A, K560R, A561T, A561G, K562R, K562N, I563L, T564I, A565S, A565V, K567R, K568N, K568R, Q569K, Q569L, Q569R, A570V, Q571R, D574N, V575M, V575A, S576R, T580I, T580A, T582I, T582S, I583V, K584R, V585M, S586P, S586A, S586F, E587A, E588K, E588G, E588D, S589I, S589R, S589N, A590S, A590T, A591V, P592L, V593M, V593A, Q594L, K595R, K595N, H596Y, H596L, H596P, I597T, I597V, N599H, D600L, D600N, D600G, D600V, N601S, N601K, S602A, S602P, S602Y, D603A, D603V, D604G, D604Y, D604N, D606A, D606V, D606Y, D607Y, D607E, D608N, A611T, E613D, R618I, T620P, and A656V, relative to SEQ ID NO: 5; and/or the third polypeptide comprises one or more amino acid substitutions of: M1L, M1V, N2S, A3T, T5P, T5A, T5S, E6D, 17S, 17V, 19F, Q11R, L12M, N14D, N14S, M21I, H22P, H22Y, K26N, K26R, T27I, M31I, L35R, N38S, S43P, D44N, D44G, Q46L, C47S, T54I, S59T, H60Y, T61A, H64Y, Y65H, K67N, K67R, R68Q, A71G, T72A, N74D, S76C, S76Y, T79I, M80I, P81S, V84L, R89L, A95D, A95T, A102T, E105D, E105K, S109N, S109R, S110P, Q111R, I112T, K113N, K113E, K114N, K114M, K114E, G116D, K118N, K118R, T119I, D120V, K123N, L129M, I130V, K131R, A132S, K134M, K134N, F142V, L145M, I146T, E147K, F148S, S150F, R154K, Q155H, E166D, K169E, P178S, A180V, A181T, A181S, I183V, A184S, A184T, A184V, P187S, A190T, A190V, V194M, V194A, R197I, Y201N, L204M, D207N, K209N, Q213H, Q213V, A219S, K221N, D225N, V226E, P227T, K229E, S232N, K233N, K233R, N234H, T236A, A238V, A238S, A241S, E246D, K251N, H252Y, H252R, E256D, A257S, A261V, S263I, S263N, N265D, Y267C, E269K, E269D, K271E, K271R, H272Y, I274V, F280L, D281N, D281G, K285G, K286N, K288R, S291F, S291P, K292N, K296R, K296N, I299S, D301G, E303D, I304T, I304V, E306G, V307L, V307G, V307A, V307D, V307G, I308N, N310S, Y313H, N314K, N316K, N316D, A317D, L318Q, D319N, P320S, P320L, M323I, L324M, D326N, V328M, V328A, A330D, I331V, V332G, S340L, T341A, A343G, S344N, I355V, F412V, V418F, Y427C, R514K, S1198L, A1201V, G1206S, C1212G, F1260L, and V1282M, relative to SEQ ID NO: 6.

In some embodiments, the second polypeptide comprises amino acid substitutions at positions: 43, 349, 352, 390, 396, 464, 549, 594 and one or more positions selected from 63, 145, 174, 182, 208, 410, 427, 456, 504, and 526; 43, 349, 352, 390, 396, 464, 549, 594, 415 and 502; 43, 349, 352, 390, 396, 464, 549, 594, 415, 502 and 67; 43, 349, 352, 390, 396, 464, 549, 594, 415, 502 and 21; or 43, 349, 352, 390, 396, 464, 549, 594, 415, 502, 21 and 67, relative to SEQ ID NO: 5; and/or the third polypeptide comprises amino acid substitutions at positions: 197, 314, and optionally one of 7, 12, or 114; 76 and 7, 12 or 263; or 76, 238, 296, or 328, relative to SEQ ID NO: 6. In some embodiments, the second polypeptide comprises substitutions of: F43S, Y349N or Y349D, P352T, A390V, D396N, H464R, Q549R, Q594L and one or more substitutions selected from R63G, A145S, A174S, I182R, V208M, Q410K, T427S, T456I or T456P, P504S, and V526E; F43S, Y349N or Y349D, P352T, A390V, D396N, H464R, Q549R, Q594L, A415V and T502I; F43S, Y349N or Y349D, P352T, A390V, D396N, H464R, Q549R, Q594L, A415V, T502I, and T21A; F43S, Y349N or Y349D, P352T, A390V, D396N, H464R, Q549R, Q594L, A415V, T502I, and Q67K; or F43S, Y349N or Y349D, P352T, A390V, D396N, H464R, Q549R, Q594L, A415V, T502I, T21A, and Q67K, relative to SEQ ID NO: 5; and/or the third polypeptide comprises substitutions of: R197I, N314K, and optionally one of I7S, L12M, or K114M; S76Y and I7V, L12M or S263N; or S76Y, A238S, K296N, or V328M relative to SEQ ID NO: 6.

In some embodiments, the first polypeptide and second polypeptide are linked in a fusion protein.

In some embodiments, the composition comprises two or more of a first polypeptide, a second polypeptide, a third polypeptide, and a fourth polypeptide.

In some embodiments, the first polypeptide comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 7 with one or more amino acid substitutions, deletions, or additions relative to SEQ ID NO: 7. In some embodiments, the second polypeptide comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 8 with one or more amino acid substitutions, deletions, or additions relative to SEQ ID NO: 8. In some embodiments, the third polypeptide comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 9 with one or more amino acid substitutions, deletions, or additions relative to SEQ ID NO: 9. In some embodiments, the fourth polypeptide comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 10 with one or more amino acid substitutions, deletions, or additions relative to SEQ ID NO: 10.

In some embodiments, the first polypeptide comprises one or more amino acid substitutions at positions: 99, 133, 189, 265, 266, 336, and 343, relative to SEQ ID NO: 7. In some embodiments, the second polypeptide comprises one or more amino acid substitutions at positions: 119, 134, 155, 180, 183, 274, 319, 447, 454, 458, 461, 512, 538, and 580, relative to SEQ ID NO: 8. In some embodiments, the third polypeptide comprises one or more amino acid substitutions at positions: 28, 82, 144, 151, 162, 182, 273, 327, and 346, relative to SEQ ID NO: 9. In some embodiments, the fourth polypeptide comprises one or more amino acid substitutions at positions: 21 and 90, relative to SEQ ID NO: 10.

In some embodiments, the first polypeptide comprises one or more amino acid substitutions of: M99I, S189N, H265Q, A266V, L336F, and V343A, relative to SEQ ID NO: 7. In some embodiments, the second polypeptide comprises one or more amino acid substitutions of: Y119H, N134R, N134Q, D155N, Q180R, D183N, R274L, N319D, V447I, A454S, E458G, D461N, A512T, D538K, and P580Q, relative to SEQ ID NO: 8. In some embodiments, the third polypeptide comprises one or more amino acid substitutions of: R28K, A82T, K144, C151R, N162S, K182E, D273G, A327D, and M346I, relative to SEQ ID NO: 9. In some embodiments, the fourth polypeptide comprises one or more amino acid substitutions of: A21S and V90A, relative to SEQ ID NO: 10.

In some embodiments, the first polypeptide comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 11 with one or more amino acid substitutions, deletions, or additions relative to SEQ ID NO: 11. In some embodiments, the second polypeptide comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 12 with one or more amino acid substitutions, deletions, or additions relative to SEQ ID NO: 12. In some embodiments, the third polypeptide comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 13 with one or more amino acid substitutions, deletions, or additions relative to SEQ ID NO: 13. In some embodiments, the fourth polypeptide comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 14 with one or more amino acid substitutions, deletions, or additions relative to SEQ ID NO: 14.

In some embodiments, the first polypeptide comprises one or more amino acid substitutions at positions: 2, 3, 7, 9, 11, 12, 14, 16, 20, 26, 29, 32, 34, 35, 40, 43, 45, 46, 54, 61, 64, 65, 70, 77, 101, 103, 105, 106, 108, 109, 111, 119, 120, 123, 126, 127, 130, 131, 148, 149, 151, 157, 159, 164, 166, 185, 194, 196, 203, 211, 217, 218, 219, 236, 242, 257, 267, 279, 283, 286, 288, 291, 293, 296, 303, 306, 313, 314, 316, 326, 331, 336, 347, 352, 361, 374, 377, 395, 396, 398, and 408, relative to SEQ ID NO: 11.

In some embodiments, the second polypeptide comprises one or more amino acid substitutions at positions: 4, 5, 6, 8, 9, 11, 12, 13, 16, 17, 20, 21, 24, 26, 28, 29, 34, 37, 38, 41, 49, 54, 59, 60, 63, 65, 67, 74, 77, 81, 88, 92, 93, 94, 96, 102, 105, 106, 108, 110, 121, 126, 128, 134, 138, 142, 147, 150, 151, 153, 156, 157, 160, 162, 165, 170, 171, 173, 174, 179, 181, 183, 185, 186, 187, 188, 191, 198, 201, 206, 207, 226, 228, 233, 236, 241, 249, 250, 256, 267, 268, 270, 275, 276, 277, 279, 283, 286, 289, 303, 305, 306, 310, 312, 314, 315, 316, 323, 326, 329, 349, 353, 355, 356, 357, 358, 361, 370, 372, 373, 376, 378, 382, 388, 391, 397, 399, 403, 405, 419, 421, 423, 424, 425, 427, 428, 430, 431, 432, 433, 449, 457, 473, 477, 480, 485, 487, 489, 494, 496, 497, 498, 500, 502, 509, 511, 515, 518, 519, 520, 540, 545, 550, 555, 557, 570, 571, 580, 583, 585, 590, 594, 603, 607, 608, 611, 617, 620, 624, 636, 639, 641, 642, 644, 646, 655, 658, 660, 663, 665, 668, 672, 673, 678, 682, 685, 688, and 695, relative to SEQ ID NO: 12.

In some embodiments, the third polypeptide comprises one or more amino acid substitutions at positions: 5, 10, 11, 26, 30, 35, 40, 42, 45, 46, 47, 58, 61, 65, 71, 72, 75, 77, 78, 80, 82, 83, 94, 98, 113, 115, 116, 117, 121, 128, 133, 138, 146, 148, 161, 171, 175, 177, 182, 184, 191, 193, 201, 203, 211, 212, 219, 225, 226, 232, 233, 235, 236, 237, 238, 240, 250, 274, 282, 286, 292, 295, 304, 307, 309, 312, 313, 315, 316, 317, 318, 320, 321, 322, 323, 328, 340, 343, 344, 345, 347, 348, 349, and 350, relative to SEQ ID NO: 13.

In some embodiments, the third polypeptide comprises an amino acid sequence having at least 70% identity to SEQ ID NO: 13 and at least one amino acid substitution with a positively charged amino acid. In some embodiments, the positively charged amino acid is arginine. In some embodiments, the at least one amino acid substitution is at position 2, 5, 6, 7, 8, 9, 10, 12, 38, 39, 40, 41, 42, 43, 44, 45, 46, 47, 48, 64, 65, 66, 67, 68, 69, 70, 222, 224, 225, 227, 228, 229, 231, 232, 233, 234, 235, 255, 256, 257, 258, 277, 286, 287, 337, 338, 339, 340, 345, 346, 347, 348, 349, 350, or a combination thereof. In some embodiments, the at least one amino acid substitution is at positions: 346 and 348; 346, 348 and 349; 346, 348, 349, an 350; 350 and 351; 350, 351, and 352; 350, 351, 352, and 353; 235 and 227; 235 and 345; 235 and 346; 235 and 347; 235 and 348; 235 and 349; 235 and 350; 235, 227, and 349; 5, 235 and 346; 5, 235 and 348; 5, 235 and 349; 227, 235, and 346; or 227, 235, and 348.

In some embodiments, the fourth polypeptide comprises one or more amino acid substitutions at positions: 2, 9, 13, 14, 15, 34, 38, 42, 46, 50, 59, 60, 73, 75, 77, 82, 83, 85, 86, 97, 110, 115, 120, 124, 130, 132, 134, 140, 143, 145, 156, 159, 162, 164, 177, 199, 232, and 270, relative to SEQ ID NO: 14.

In some embodiments, the first polypeptide comprises one or more amino acid substitutions of: A2T, F3S, P7R, A9S, A9G, A11G, F12I, D14N, S16Y, Y20H, S26N, F29S, S32N, E34K, G35V, G35S, G35D, I40S, E43D, H45P, E46K, A54S, R61W, V64M, Y65C, N70S, A77T, D101N, K103E, N105K, N105D, S106G, V108M, A109G, Y111N, L119M, R120S, R123S, A126T, E127G, V130M, D131N, Q148R, S149Y, H151Y, A157D, T159I, A164V, L166M, T185A, S194G, A196T, T203A, K211R, E217K, R218K, R218S, N219S, A236T, E242D, N257K, N267S, M279I, M279V, D283G, N286S, T288I, K291Q, I293V, D296N, S303I, S303G, K306N, S310Y, S310P, I313T, Y314F, A316T, E326G, T331I, A336V, A347T, A347S, T352S, Y361H, M374T, M374I, R377G, T395I, S396T, S396F, G398V, and A408V, relative to SEQ ID NO: 11. In some embodiments, the second polypeptide comprises one or more amino acid substitutions of: K4N, E5K, L6M, L6I, E8K, E8D, 19T, D11N, T12A, T13I, D16G, R17C, R17S, R20K, R20E, R21E, R21K, S24K, S24Q, S24R, Y26S, Y26H, A28S, A28D, M29I, G34D, A37S, V38M, V38G, I41V, R49L, D54G, K59R, K60N, K63N, A65T, A65V, K67E, K74E, K77E, W81C, K88R, K88E, 192T, R93E, R93K, V94M, K96N, E102D, E102G, T105A, L106M, S108P, V110A, G121S, S126P, K128R, L134M, Y138S, Q142H, W147L, K150N, V151M, V151L, A153T, S156R, S156G, D157N, K160R, K160E, A162T, S165N, S165G, V170E, K171E, F173V, K174N, K174R, T179A, K181T, S183N, P185T, E186K, E186D, E187K, A188S, A188V, D191Y, D191E, R198H, R198C, R198S, R201K, D206G, G207D, A226T, I228V, R233K, N236T, R241E, A249S, A250S, I256T, S267G, S267N, K268N, H270P, S275N, S275G, R276G, A277D, A277S, A277T, K279N, G283D, V286G, V289M, G303D, I305T, F306S, A310D, A310T, A312G, A312D, A312T, K314N, Q315R, R316G, N323S, E326A, E326K, N329S, G349D, E353D, L355M, L355R, E356G, E356D, S357P, A358V, R361S, P370T, N372K, E373D, S376F, T378I, F382L, M388V, G391S, R397K, A399S, K403N, M405I, L419P, D421N, K423R, H424N, H424R, V425L, I427V, E428K, D430A, D431G, E432D, H433N, A449T, G457D, R473K, E477D, G480D, F485L, S487R, S487G, S489N, N494D, S496N, A497S, V498G, K500N, K502N, Q509R, A511T, A511E, R515S, R518S, P519T, G520D, G520V, Y540C, Q545H, K550N, K555E, H557Q, P570S, E571D, C580R, S583R, E585K, E585G, E590D, R594K, M603I, H607N, H607L, K608R, D611N, L617P, N620S, K624N, T636P, M639V, N641S, V642G, S644N, S644G, E646D, A655V, V658M, K660N, T663A, T665I, R668S, I672V, G673V, S678R, M682L, A685V, A685D, K688N, and V695M, relative to SEQ ID NO: 12. In some embodiments, the third polypeptide comprises one or more amino acid substitutions of: N5K, N5T, DION, R11K, D26N, V30E, D35N, R40L, P42A, G45S, G45V, F46V, T47R, T47S, N58T, P61L, T65I, T71I, T71R, T71D, L72M, C75S, V77A, P78L, N80T, E82D, H83Y, H83N, A94S, V98M, E113D, C121F, A128S, A155S, E116D, T117I, R133K, G138V, N146D, G148V, C161R, A171V, A171S, K175T, A177V, K182E, L184M, I191V, S193A, S193F, F201S, S203N, E211K, A212V, Y219R, N225S, N225T, D226Y, E232K, E232Q, A233N, A233S, A233K, K235R, Q236R, Q236S, F237L, V238Q, V238M, A240T, A240V, S250A, R274G, A282V, I286N, I286T, I286F, P292S, S295N, K304R, E307D, Y309C, A312V, L313M, N315K, N315T, N315S, C316G, I317V, T318A, T318P, K320R, N321D, E322K, K323N, I328T, M340I, K343E, K343R, K344E, K344R, A345T, A345D, A345S, A345Y, A345R, A345K, A345E, A345G, A347K, A347S, A347D, K348N, K349R, A350K, A350D, A350V, and A350T, relative to SEQ ID NO: 13. In some embodiments, the fourth polypeptide comprises one or more amino acid substitutions of: Q2K, H9L, K13E, Q14K, A15G, K34N, E38K, V42I, A46D, S50I, V59G, Y60H, A73S, A73T, F75L, D77G, G82S, F83L, F83V, F83C, K85E, V86I, E97, I110S, I110L, S115R, K120N, K124R, G130D, D132E, N134T, A140T, E143K, D145G, S156I, E159K, I162V, H164Y, H164F, Y177C, S199I, S232L, and L270S, relative to SEQ ID NO: 14.

In some embodiments, the first polypeptide comprises one or more amino acid substitutions at positions: 105, 109, 131, 148, 279, and 310; or 9, 105, 109, 131, 148, 279, and 310, relative to SEQ ID NO: 11. In some embodiments, the second polypeptide comprises one or more amino acid substitutions at positions: 134, 179, 185, 540, 555, 624, and 646; 138, 250, 275, and 421; 303, 405, 520, and 590; 134, 179, 185, 540, 555, and 646, 4 and 49; 4 and 388; 4 and 571; 4, 162, and 480; 4 and 315; 5 and 316; 17 and 156; 38, 108, 497 and 583; 59, 157 and 644; 96, 305, 550 and 642; 106, 160 and 228; 312, 424, 449 and 457; or 376 and 611, relative to SEQ ID NO: 12. In some embodiments, the third polypeptide comprises one or more amino acid substitutions at positions: 30, 46, 240, 304, and 316; 30, 46, 240, and 316; 42 and 318; 184, 240, 315, and 345; 211 and 274; 237 and 237; 286 and 350; 317 and 347; 171, 286, and 315; or 328 and 350, relative to SEQ ID NO: 13. In some embodiments, the fourth polypeptide comprises one or more amino acid substitutions at positions: 82, 110, 115, 164, and 199; 82, 110, 115, 124, 164, and 199; 110, 115, and 164; 110, 115, 164, and 199; 110, 115, 164, 199, and 124; or 110, 115, 164, 199, and 82 or 124, relative to SEQ ID NO: 14.

In some embodiments, the composition further comprises one or more Cas proteins. In some embodiments, the one or more Cas proteins are selected from the group consisting of Cas5, Cas6, Cas7, Cas8, Cas9, Cas 11, Cas12, and variants thereof.

In some embodiments, the composition further comprises at least one unfoldase protein. In some embodiments, the at least one unfoldase protein comprises ClpX.

Further provided herein are systems comprising an engineered Clustered Regularly Interspaced Short Palindromic Repeats (CRISPR)-associated transposon (CRISPR-Tn or CAST) system or one or more nucleic acids encoding the engineered CRISPR-Tn system. In some embodiments, the CRISPR-Tn system comprises at least one or both of: a) one or more Cas proteins selected from: Cas5, Cas6, Cas7, Cas8, Cas9, Cas11, and combinations thereof; and b) one or more transposon-associated proteins selected from TnsA, TnsB, TnsC, TnsD, TniQ, and combinations thereof. In some embodiments, at least one of the one or more Cas protein comprises Cas6, Cas7 or Cas8 as described herein or at least one of the one or more transposon-associated proteins comprises TnsA, TnsB, TnsC, or TniQ as described herein.

In some embodiments, at least one of the one or more Cas protein comprises: a Cas6 protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 10 or 14 with one or more amino acid substitutions, deletions, or additions relative to SEQ ID NO: 10 or 14; a Cas7 protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 9 or 13 with one or more amino acid substitutions, deletions, or additions relative to SEQ ID NO: 9 or 13; or a Cas8-Cas5 fusion protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 8 or 12 with one or more amino acid substitutions, deletions, or additions relative to SEQ ID NO: 8 or 12. In some embodiments, at least one of the one or more transposon-associated proteins comprises: a TnsA protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 1 or 4 with one or more amino acid substitutions, deletions, or additions relative to SEQ ID NO: 1 or 4; a TnsB protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 2 or 5 with one or more amino acid substitutions, deletions, or additions relative to SEQ ID NO: 2 or 5; a TnsC protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 3 or 6 with one or more amino acid substitutions, deletions, or additions relative to SEQ ID NO: 3 or 6, or a TniQ protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 7 or 11 with one or more amino acid substitutions, deletions, or additions relative to SEQ ID NO: 7 or 11.

In some embodiments, the TniQ protein comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 7 with one or more amino acid substitutions, deletions, or additions relative to SEQ ID NO: 7. In some embodiments the Cas6 protein comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 10 with one or more amino acid substitutions, deletions, or additions relative to SEQ ID NO: 10; the Cas7 protein comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 9 with one or more amino acid substitutions, deletions, or additions relative to SEQ ID NO: 9; and/or the Cas8-Cas5 fusion protein comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: with one or more amino acid substitutions, deletions, or additions relative to SEQ ID NO: 8.

In some embodiments, the TniQ protein comprises an amino acid sequence having one or more amino acid substitutions at positions: 99, 133, 189, 265, 266, 336, and 343, relative to SEQ ID NO: 7. In some embodiments, the Cas6 protein comprises an amino acid having one or more amino acid substitutions at positions: 21 and 90, relative to SEQ ID NO: 10. In some embodiments, the Cas7 protein comprises an amino acid having one or more amino acid substitutions at positions: 28, 82, 144, 151, 162, 182, 273, 327, 346, relative to SEQ ID NO: 9. In some embodiments, the Cas8-Cas5 fusion protein comprises an amino acid having one or more amino acid substitutions at positions: 119, 134, 155, 180, 183, 274, 319, 447, 454, 458, 461, 512, 538, and 580, relative to SEQ ID NO: 8.

In some embodiments, the TniQ protein comprises an amino acid sequence having one or more amino acid substitutions of: M991, S189N, H265Q, A266V, L336F, and V343A, relative to SEQ ID NO: 7. In some embodiments, the Cas6 protein comprises an amino acid sequence having one or more amino acid substitutions of: A21S and V90A, relative to SEQ ID NO: 10. In some embodiments, the Cas7 protein comprises an amino acid sequence having one or more amino acid substitutions of: R28K, A82T, K144, C151R, N162S, K182E, D273G, A327D, and M346I, relative to SEQ ID NO: 9. In some embodiments, the Cas8-Cas5 fusion protein comprises an amino acid sequence having one or more amino acid substitutions of: Y119H, N134R, N134Q, D155N, Q180R, D183N, R274L, N319D, V447I, A454S, E458G, D461N, A512T, D538K, and P580Q, relative to SEQ ID NO: 8.

In some embodiments, the TniQ protein comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 11 with one or more amino acid substitutions, deletions, or additions relative to SEQ ID NO: 11. In some embodiments, the Cas6 protein comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 14 with one or more amino acid substitutions, deletions, or additions relative to SEQ ID NO: 14. In some embodiments, the Cas7 protein comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 13 with one or more amino acid substitutions, deletions, or additions relative to SEQ ID NO: 13. In some embodiments, the Cas8-Cas5 fusion protein comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 12 with one or more amino acid substitutions, deletions, or additions relative to SEQ ID NO: 12.

In some embodiments, the TniQ protein comprises an amino acid sequence having one or more amino acid substitutions at positions: 2, 3, 7, 9, 11, 12, 14, 16, 20, 26, 29, 32, 34, 35, 40, 43, 45, 46, 54, 61, 64, 65, 70, 77, 101, 103, 105, 106, 108, 109, 111, 119, 120, 123, 126, 127, 130, 131, 148, 149, 151, 157, 159, 164, 166, 185, 194, 196, 203, 211, 217, 218, 219, 236, 242, 257, 267, 279, 283, 286, 288, 291, 293, 296, 303, 306, 313, 314, 316, 326, 331, 336, 347, 352, 361, 374, 377, 395, 396, 398, and 408, relative to SEQ ID NO: 11. In some embodiments, the Cas6 protein comprises an amino acid sequence having one or more amino acid substitutions at positions: 2, 9, 13, 14, 15, 34, 38, 42, 46, 50, 59, 60, 73, 75, 77, 82, 83, 85, 86, 97, 110, 115, 120, 124, 130, 132, 134, 140, 143, 145, 156, 159, 162, 164, 177, 199, 232, and 270, relative to SEQ ID NO: 14. In some embodiments, the Cas7 protein comprises an amino acid sequence having one or more amino acid substitutions at positions: 5, 10, 11, 26, 30, 35, 40, 42, 45, 46, 47, 58, 61, 65, 71, 72, 75, 77, 78, 80, 82, 83, 94, 98, 113, 115, 116, 117, 121, 128, 133, 138, 146, 148, 161, 171, 175, 177, 182, 184, 191, 193, 201, 203, 211, 212, 219, 225, 226, 232, 233, 235, 236, 237, 238, 240, 250, 274, 282, 286, 292, 295, 304, 307, 309, 312, 313, 315, 316, 317, 318, 320, 321, 322, 323, 328, 340, 343, 344, 345, 347, 348, 349, and 350, relative to SEQ ID NO: 13. In some embodiments, the Cas8-Cas5 fusion protein comprises an amino acid sequence having one or more amino acid substitutions at positions: 4, 5, 6, 8, 9, 11, 12, 13, 16, 17, 20, 21, 24, 26, 28, 29, 34, 37, 38, 41, 49, 54, 59, 60, 63, 65, 67, 74, 77, 81, 88, 92, 93, 94, 96, 102, 105, 106, 108, 110, 121, 126, 128, 134, 138, 142, 147, 150, 151, 153, 156, 157, 160, 162, 165, 170, 171, 173, 174, 179, 181, 183, 185, 186, 187, 188, 191, 198, 201, 206, 207, 226, 228, 233, 236, 241, 249, 250, 256, 267, 268, 270, 275, 276, 277, 279, 283, 286, 289, 303, 305, 306, 310, 312, 314, 315, 316, 323, 326, 329, 349, 353, 355, 356, 357, 358, 361, 370, 372, 373, 376, 378, 382, 388, 391, 397, 399, 403, 405, 419, 421, 423, 424, 425, 427, 428, 430, 431, 432, 433, 449, 457, 473, 477, 480, 485, 487, 489, 494, 496, 497, 498, 500, 502, 509, 511, 515, 518, 519, 520, 540, 545, 550, 555, 557, 570, 571, 580, 583, 585, 590, 594, 603, 607, 608, 611, 617, 620, 624, 636, 639, 641, 642, 644, 646, 655, 658, 660, 663, 665, 668, 672, 673, 678, 682, 685, 688, and 695, relative to SEQ ID NO: 12.

In some embodiments, the TniQ protein comprises an amino acid sequence having one or more amino acid substitutions of: A2T, F3S, P7R, A9S, A9G, A11G, F12I, D14N, S16Y, Y20H, S26N, F29S, S32N, E34K, G35V, G35S, G35D, I40S, E43D, H45P, E46K, A54S, R61W, V64M, Y65C, N70S, A77T, D101N, K103E, N105K, N105D, S106G, V108M, A109G, Y111N, L119M, R120S, R123S, A126T, E127G, V130M, D131N, Q148R, S149Y, H151Y, A157D, T159I, A164V, L166M, T185A, S194G, A196T, T203A, K211R, E217K, R218K, R218S, N219S, A236T, E242D, N257K, N267S, M279I, M279V, D283G, N286S, T288I,

K291Q, I293V, D296N, S303I, S303G, K306N, S310Y, S310P, I313T, Y314F, A316T, E326G, T331I, A336V, A347T, A347S, T352S, Y361H, M374T, M374I, R377G, T395I, S396T, S396F, G398V, and A408V, relative to SEQ ID NO: 11. In some embodiments, the Cas6 protein comprises an amino acid sequence having one or more amino acid substitutions of: Q2K, H9L, K13E, Q14K, A15G, K34N, E38K, V421, A46D, S50I, V59G, Y60H, A73S, A73T, F75L, D77G, G82S, F83L, F83V, F83C, K85E, V86I, E97, I110S, I110L, S115R, K120N, K124R, G130D, D132E, N134T, A140T, E143K, D145G, S156I, E159K, I162V, H164Y, H164F, Y177C, S199I, S232L, and L270S, relative to SEQ ID NO: 14. In some embodiments, the Cas7 protein comprises an amino acid sequence having one or more amino acid substitutions of: N5K, N5T, DION, R11K, D26N, V30E, D35N, R40L, P42A, G45S, G45V, F46V, T47R, T47S, N58T, P61L, T65I, T71I, T71R, T71D, L72M, C75S, V77A, P78L, N80T, E82D, H83Y, H83N, A94S, V98M, E113D, C121F, A128S, A155S, E116D, T117I, R133K, G138V, N146D, G148V, C161R, A171V, A171S, K175T, A177V, K182E, L184M, I191V, S193A, S193F, F201S, S203N, E211K, A212V, Y219R, N225S, N225T, D226Y, E232K, E232Q, A233N, A233S, A233K, K235R, Q236R, Q236S, F237L, V238Q, V238M, A240T, A240V, S250A, R274G, A282V, I286N, I286T, I286F, P292S, S295N, K304R, E307D, Y309C, A312V, L313M, N315K, N315T, N315S, C316G, I317V, T318A, T318P, K320R, N321D, E322K, K323N, I328T, M340I, K343E, K343R, K344E, K344R, A345T, A345D, A345S, A345Y, A345R, A345K, A345E, A345G, A347K, A347S, A347D, K348N, K349R, A350K, A350D, A350V, and A350T, relative to SEQ ID NO: 13. In some embodiments, the Cas8-Cas5 fusion protein comprises an amino acid sequence having one or more amino acid substitutions of: K4N, E5K, L6M, L6I, E8K, E8D, 19T, D11N, T12A, T13I, D16G, R17C, R17S, R20K, R20E, R21E, R21K, S24K, S24Q, S24R, Y26S, Y26H, A28S, A28D, M29I, G34D, A37S, V38M, V38G, I41V, R49L, D54G, K59R, K60N, K63N, A65T, A65V, K67E, K74E, K77E, W81C, K88R, K88E, 192T, R93E, R93K, V94M, K96N, E102D, E102G, T105A, L106M, S108P, V110A, G121S, S126P, K128R, L134M, Y138S, Q142H, W147L, K150N, V151M, V151L, A153T, S156R, S156G, D157N, K160R, K160E, A162T, S165N, S165G, V170E, K171E, F173V, K174N, K174R, T179A, K181T, S183N, P185T, E186K, E186D, E187K, A188S, A188V, D191Y, D191E, R198H, R198C, R198S, R201K, D206G, G207D, A226T, I228V, R233K, N236T, R241E, A249S, A250S, I256T, S267G, S267N, K268N, H270P, S275N, S275G, R276G, A277D, A277S, A277T, K279N, G283D, V286G, V289M, G303D, I305T, F306S, A310D, A310T, A312G, A312D, A312T, K314N, Q315R, R316G, N323S, E326A, E326K, N329S, G349D, E353D, L355M, L355R, E356G, E356D, S357P, A358V, R361S, P370T, N372K, E373D, S376F, T378I, F382L, M388V, G391S, R397K, A399S, K403N, M405I, L419P, D421N, K423R, H424N, H424R, V425L, I427V, E428K, D430A, D431G, E432D, H433N, A449T, G457D, R473K, E477D, G480D, F485L, S487R, S487G, S489N, N494D, S496N, A497S, V498G, K500N, K502N, Q509R, A511T, A511E, R515S, R518S, P519T, G520D, G520V, Y540C, Q545H, K550N, K555E, H557Q, P570S, E571D, C580R, S583R, E585K, E585G, E590D, R594K, M603I, H607N, H607L, K608R, D611N, L617P, N620S, K624N, T636P, M639V, N641S, V642G, S644N, S644G, E646D, A655V, V658M, K660N, T663A, T665I, R668S, I672V, G673V, S678R, M682L, A685V, A685D, K688N, and V695M, relative to SEQ ID NO: 12.

In some embodiments, the TniQ protein comprises an amino acid sequence having amino acid substitutions at positions: 105, 109, 131, 148, 279, and 310; or 9, 105, 109, 131, 148, 279, and 310, relative to SEQ ID NO: 11. In some embodiments, the Cas6 protein comprises an amino acid sequence having amino acid substitutions at positions: 110, 115, 164, 199, and 82 or 124, relative to SEQ ID NO: 14. In some embodiments, the Cas7 protein comprises an amino acid sequence having amino acid substitutions at positions: 30, 46, 240, 304, and 316; 30, 46, 240, and 316; 42 and 318; 184, 240, 315, and 345; 211 and 274; 237 and 237; 286 and 350; 317 and 347; 171, 286, and 315; or 328 and 350, relative to SEQ ID NO: 13. In some embodiments, the Cas8-Cas5 fusion protein comprises an amino acid sequence having amino acid substitutions at positions: 134, 179, 185, 540, 555, 624, and 646; 138, 250, 275, and 421; 303, 405, 520, and 590; 134, 179, 185, 540, 555, and 646, 4 and 49; 4 and 388; 4 and 571; 4, 162, and 480; 4 and 315; 5 and 316; 17 and 156; 38, 108, 497 and 583; 59, 157 and 644; 96, 305, 550 and 642; 106, 160 and 228; 312, 424, 449 and 457; or 376 and 611, relative to SEQ ID NO: 12.

In some embodiments, the Cas7 protein comprises an amino acid sequence having at least 70% identity to SEQ ID NO: 13 and at least one amino acid substitution with a positively charged amino acid. In some embodiments, the positively charged amino acid is arginine. In some embodiments, the at least one amino acid substitution is at position 2, 5, 6, 7, 8, 9, 10, 12, 38, 39, 40, 41, 42, 43, 44, 45, 46, 47, 48, 64, 65, 66, 67, 68, 69, 70, 222, 224, 225, 227, 228, 229, 231, 232, 233, 234, 235, 255, 256, 257, 258, 277, 286, 287, 337, 338, 339, 340, 345, 346, 347, 348, 349, 350, or a combination thereof. In some embodiments, the at least one amino acid substitution is at positions: 346 and 348; 346, 348 and 349; 346, 348, 349, an 350; 350 and 351; 350, 351, and 352; 350, 351, 352, and 353; 235 and 227; 235 and 345; 235 and 346; 235 and 347; 235 and 348; 235 and 349; 235 and 350; 235, 227, and 349; 5, 235 and 346; 5, 235 and 348; 5, 235 and 349; 227, 235, and 346; or 227, 235, and 348.

In some embodiments, the system comprises a TnsA protein and TnsB protein. In some embodiments, the TnsA protein comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 1 with one or more amino acid substitutions, deletions, or additions relative to SEQ ID NO: 1. In some embodiments, the TnsB protein comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 2 with one or more amino acid substitutions, deletions, or additions relative to SEQ ID NO: 2.

In some embodiments, the TnsA protein comprises an amino acid sequence having one or more amino acid substitutions at positions: 2, 3, 5, 28, 57, 77, 80, 107, 110, 116, 122, 142, 155, 161, 166, 173, 177, 185, 211, 216, 227, and 230, relative to SEQ ID NO: 1. In some embodiments, TnsB protein comprises an amino acid sequence having one or more amino acid substitutions at positions: 2, 5, 22, 24, 25, 29, 75, 141, 199, 215, 319, 347, 364, 370, 383, 439, 454, 458, 485, 509, 533, 538, 565, 581, 586, 595, 596, 597, and 600, relative to SEQ ID NO: 2.

In some embodiments, the TnsA protein comprises an amino acid sequence having one or more amino acid substitutions of: A2T, T3I, L5S, T28A, A57T, F77L, Y80D, K107M, K107R, Y110C, Y110D, D116G, E122A, D142E, M155I, K161R, N166D, K173E, Y177N, Y177D, C185R, D211Y, K216E, A227P, G230D, and G230S, relative to SEQ ID NO: 1. In some embodiments, the TnsB protein comprises an amino acid sequence having one or more amino acid substitutions of: A2T, A2S, G5R, S22P, E24D, L25I, A29S, P75T, I141T, V199I, S215R, D319V, Y347F, S364N, E370K, N383D, V439A, E454D, E454G, S458N, V485F, R509G, D533A, A538V, H565Y, A581T, H586L, N595K, D596N, D597N, D597Y, and I600V, relative to SEQ ID NO: 2.

In some embodiments, the TnsA protein comprises an amino acid sequence having amino acid substitutions at positions: 2 and 230; 107 and 166; 107, 166, and one or both of: 2 and 227; 211 and 110 or 142; 110, 155 and 230; 122 and 155; or 155 and 177, relative to SEQ ID NO: 1. In some embodiments, the TnsB protein comprises an amino acid sequence having amino acid substitutions at positions: 2 and 597; 24 and 25; 24, 25, 458, 509, 565, and 600; 75 and 597; 141, 454, 533 and 595; 581, 370, and 454; 370 and 581; 370 and 454; 458 and 509; 458, 509 and 565; 458, 509, 565, and 600; 565, 586, and 596; or 565, 509, 458, 600 and at least one of 24, 25, 29, 215, 319, 364, 383, and 586, relative to SEQ ID NO: 2.

In some embodiments, the TnsA protein comprises an amino acid sequence having amino acid substitutions at positions: 107, 166, and 227, relative to SEQ ID NO: 1 and the TnsB protein comprises an amino acid sequence having amino acid substitutions at positions: 24, 25, 458, 509, 565, and 600, relative to SEQ ID NO: 2; the TnsA protein comprises an amino acid sequence having an amino acid substitution at position 155, relative to SEQ ID NO: 1, and the TnsB protein comprises an amino acid sequence having amino acid substitutions at positions: 22, 347, and 454, relative to SEQ ID NO: 2; or the TnsA protein comprises an amino acid sequence having amino acid substitutions at positions: 122 and 155, relative to SEQ ID NO: 1, and the TnsB protein comprises an amino acid sequence having an amino acid substitution at position: 485, relative to SEQ ID NO: 2.

In some embodiments, the TnsA protein comprises an amino acid sequence having amino acid substitutions: K107M, N166D, and A227P, relative to SEQ ID NO: 1 and the TnsB protein comprises an amino acid sequence having amino acid substitutions: E24D, L25I, S458N, R509G, H565Y, and I600V, relative to SEQ ID NO: 2; the TnsA protein comprises an amino acid sequence having amino acid substitution: M155I, relative to SEQ ID NO: 1, and the TnsB protein comprises an amino acid sequence having amino acid substitutions: S22P, Y347F, and E454G, relative to SEQ ID NO: 2; or the TnsA protein comprises an amino acid sequence having amino acid substitutions: E122A and M155I, relative to SEQ ID NO: 1, and the TnsB protein comprises an amino acid sequence having amino acid substitution: V485F, relative to SEQ ID NO: 2.

In some embodiments, the system further comprises a TnsC protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 3 with one or more amino acid substitutions, deletions, or additions relative to SEQ ID NO: 3. In some embodiments, the TnsC protein comprises an amino acid sequence having one or more amino acid substitutions at positions: 9, 15, 16, 18, 21, 64, 81, 86, 87, 99, 109, 142, 147, 153, 168, 180, 216, 230, 285, and 304, relative to SEQ ID NO: 3. In some embodiments, the TnsC protein comprises an amino acid sequence having one or more amino acid substitutions of: 19V, A15V, F16Y, S18F, S21N, N64D, H81Y, D86Y, N87K, V99I, E109D, E142K, V147I, N153D, I168M, A180E, A216S, L230F, K285E, and R304R, relative to SEQ ID NO: 3. In some embodiments, the TnsC protein comprises an amino acid sequence having amino acid substitutions at positions: 142 and 216, relative to SEQ ID NO: 3.

In some embodiments, the TnsA protein comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 4 with one or more amino acid substitutions, deletions, or additions relative to SEQ ID NO: 4. In some embodiments, the TnsB protein comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 5 with one or more amino acid substitutions, deletions, or additions relative to SEQ ID NO: 5.

In some embodiments, the TnsA protein comprises an amino acid sequence having one or more amino acid substitutions at positions: 4, 5, 9, 10, 12, 21, 23, 25, 26, 31, 32, 34, 35, 37, 41, 45, 47, 48, 51, 52, 55, 60, 61, 65, 67, 69, 72, 75, 79, 80, 82, 87, 88, 90, 91, 93, 94, 96, 98, 99, 100, 103, 106, 108, 113, 116, 125, 126, 128, 129, 135, 139, 143, 146, 147, 149, 153, 154, 156, 158, 159, 160, 162, 164, 166, 167, 168, 169, 170, 177, 179, 180, 182, 183, 185, 187, 188, 190, 191, 192, 193, 195, 196, 200, 204, 207, and 208, relative to SEQ ID NO: 4. In some embodiments, the TnsB protein comprises an amino acid sequence having one or more amino acid substitutions at positions: 1, 2, 4, 5, 8, 9, 10, 11, 12, 13, 14, 15, 16, 17, 19, 20, 21, 22, 23, 24, 25, 26, 27, 28, 29, 30, 32, 33, 36, 37, 39, 40, 41, 42, 43, 44, 45, 49, 52, 55, 56, 58, 60, 62, 63, 67, 71, 74, 76, 78, 79, 80, 81, 82, 83, 84, 85, 86, 87, 88, 89, 91, 92, 95, 97, 100, 101, 104, 106, 110, 112, 113, 115, 117, 119, 120, 124, 125, 127, 129, 130, 131, 134, 139, 142, 144, 145, 146, 147, 149, 150, 155, 156, 157, 158, 159, 163, 164, 165, 167, 169, 173, 174, 176, 181, 182, 186, 187, 190, 195, 197, 198, 205, 208, 209, 211, 215, 218, 223, 226, 227, 231, 232, 235, 239, 246, 248, 250, 259, 260, 261, 262, 263, 267, 269, 273, 274, 277, 278, 280, 281, 282, 283, 285, 287, 288, 290, 295, 298, 302, 303, 307, 313, 316, 317, 320, 323, 325, 331, 332, 339, 345, 348, 349, 352, 353, 354, 356, 361, 362, 363, 364, 365, 366, 367, 369, 370, 371, 372, 373, 375, 376, 380, 383, 385, 386, 389, 390, 392, 396, 397, 399, 402, 403, 404, 407, 408, 410, 411, 412, 413, 414, 446, 448, 450, 452, 456, 459, 460, 463, 464, 470, 472, 473, 494, 495, 498, 501, 502, 504, 505, 506, 508, 509, 510, 512, 513, 514, 517, 520, 521, 522, 525, 526, 527, 530, 531, 532, 533, 535, 537, 538, 540, 541, 542, 543, 544, 545, 546, 547, 548, 549, 550, 551, 552, 553, 554, 556, 557, 558, 559, 560, 561, 562, 563, 564, 565, 567, 568, 569, 570, 571, 574, 575, 576, 580, 582, 583, 584, 585, 586, 587, 588, 589, 590, 591, 592, 593, 594, 595, 596, 597, 599, 600, 601, 602, 603, 604, 606, 607, 608, 611, 613, 618, 620, and 656, relative to SEQ ID NO: 5.

In some embodiments, the TnsA protein comprises an amino acid sequence having one or more amino acid substitutions of: R4K, N5K, P9S, A10P, N12D, T21I, V23M, S25N, S25R, V26M, V26G, S31N, S321, E34A, F35L, A37D, H41L, D45N, I47V, E48G, G51V, S52I, E55K, E55D, E60K, F61L, S65T, S65A, P67T, P67L, P67S, P67H, T69A, A72V, A72D, S75I, S75R, S75T, K79E, T80P, K82E, K87R, P88L, P88T, P88A, S90F, K91N, K91E, A93T, A93S, S94N, L96P, R98Q, A99D, A99V, E100K, A103T, A106T, S108A, I113F, V116F, V116I, V125M, V125A, N126T, I128V, I128L, L129P, L135M, S139N, S139G, G143V, G143C, G146D, G146S, I147V, K149E, K149T, K149R, S153I, S153R, S153N, F154C, H156R, H156L, S158N, S158R, G159V, V160A, K162R, N164D, I166L, S167I, S168I, S168R, S168N, Q169R, V170M, V170G, V170L, T177I, T177A, S179R, F180C, F180L, F182C, F182L, G183S, M185I, K187R, G188D, V190I, K191N, A192S, D193N, G195V, G195D, G195S, C196W, T200A, T204I, A207V, A207T, and T208I, relative to SEQ ID NO: 4. In some embodiments, the TnsB protein comprises an amino acid sequence having one or more amino acid substitutions of: M1V, M1I, M1L, T2I, T2A, F4L, F5L, F8L, F8V, F8S, D9N, E10K, E10D, S11I, S11R, S11G, L12P, V13M, V13G, V13E, V13L, P14L, L15Q, K16N, K16R, P17T, P17L, P17S, T19I, T19S, T19A, T19P, P20S, P20L, T21A, Q22R, Y23H, V24M, K25R, L26M, D27A, D27G, D28N, D28Y, A29T, A29V, N30K, I32F, I32S, Q33H, L36M, D37A, D37Y, F39L, S40P, D41E, T42I, T42K, T42A, F43L, F43S, F43V, K44N, N45D, N45S, Q49R, K52Q, S55A, T56A, D58E, K60Q, S62T, R63K, R63G, Q67R, Q67H, Q67K, D71Y, K74R, E76K, F78C, K79R, G80V, G80D, G81S, G81V, G81D, D82N, V83G, V83M, V83A, V84A, V84G, R85G, R85K, P86L, N87S, R89C, V91G, V91A, A92V, A92T, R95K, K97R, E100D, S101A, D104V, A106D, A106T, D110N, N112H, H113Y, M115R, N117Y, T119A, N120D, N120K, N120S, G124V, D125N, D125E, K127R, F129L, D130N, K131M, E134D, E134G, A139S, A139T, P142S, I144V, A145S, A145T, T146A, A147V, Q149R, Y150H, I155L, V156A, V156L, V156M, K157V, E158A, N159S, V163G, E164A, E164G, E164D, G165D, I167V, I169L, I169T, N173S, N173H, N173T, A174S, A174T, N176D, A181S, I182L, I182V, I182T, A186E, A186T, V187G, V187A, A190T, A190S, F195S, A197P, D198G, D198N, A205S, V208M, P209T, T211I, E215D, E218D, P223S, P223H, L226V, I227V, D231N, E232K, I235V, I235T, R239G, I246V, V248E, V248M, S250I, S259N, Y260C, K261R, S262N, P263L, S267N, A269V, T273I, T273N, H274Y, K277N, K277R, P278S, S280T, L281M, D282E, D282N, A283T, A283S, N285S, E287D, L288M, N290K, F295S, F298I, F298S, V302I, V303M, A307S, N313S, H316R, A317V, S320N, S320R, I323L, I325V, R331K, K332E, I339V, V345L, V345M, E348K, Y349H, Y349D, Y349N, Y349C, P352S, P352T, E353Q, E353D, L354M, G356S, N361D, I362V, I362T, L363P, L363T, L363M, E364G, K365R, E366G, E367G, K369N, K369E, K369M, P370S, E371K, V372M, D373G, I375V, M376I, T380P, T380A, E383K, E383D, F385L, H386Y, I389V, A390V, A390I, V392I, D396N, D396G, D396K, S397P, S399N, S399G, T402I, R403G, R403I, R403K, R403S, I404T, I404V, K407R, K407E, R408K, Q410K, Q410H, Q410R, Q411H, G412V, F413L, D414N, A415V, A415T, Y416C, M421I, N422K, E423K, E423D, E424A, E425K, E426D, T427A, T427S, R428K, F429L, S430A, M431L, R434H, R434C, R434S, I435V, D437G, D437N, T440S, T440I, R443C, G445S, F446L, F446I, Y448C, E450D, E450G, M452I, T456P, T456A, T456I, A459T, D460N, K463N, H464N, H464R, H464S, E470K, V472M, V472A, K473D, K473N, E494D, E494G, S495A, E498A, E498K, C501Y, T502I, T502S, P504S, P504L, T505A, G506Y, G506D, G506L, G506S, T508A, D509E, D509Y, C510Y, S512N, I513L, I513V, I513F, Y514H, K517M, K517N, K517Q, K520R, K521N, I522T, I522V, I522F, E525K, V526E, V526M, I527V, S530N, S530R, K531T, D532G, D532Y, S533Y, G535D, A537T, K538R, K538N, R540K, R540G, M541L, A542T, I543L, H544R, E545A, R546G, R546K, V547M, K548Q, K548R, Q549K, Q549R, E550A, Q551K, E552D, E552K, V553I, F554V, E556K, E556G, S557A, K558R, T559P, T559I, T559A, K560R, A561T, A561G, K562R, K562N, I563L, T564I, A565S, A565V, K567R, K568N, K568R, Q569K, Q569L, Q569R, A570V, Q571R, D574N, V575M, V575A, S576R, T580I, T580A, T582I, T582S, I583V, K584R, V585M, S586P, S586A, S586F, E587A, E588K, E588G, E588D, S589I, S589R, S589N, A590S, A590T, A591V, P592L, V593M, V593A, Q594L, K595R, K595N, H596Y, H596L, H596P, I597T, I597V, N599H, D600L, D600N, D600G, D600V, N601S, N601K, S602A, S602P, S602Y, D603A, D603V, D604G, D604Y, D604N, D606A, D606V, D606Y, D607Y, D607E, D608N, A611T, E613D, R618I, T620P, and A656V, relative to SEQ ID NO: 5.

In some embodiments, the TnsA protein comprises an amino acid sequence having amino acid substitutions at positions: 108 and 47 or 208; 170 and 207; 88 and 147; 47, 88 and 147; 88, 147, 170, and 182; 88, 147, 170, 182, and 51 or 180; 88, 147, and 154; 88, 128, 147, 170, and 182; or 170, 207, and 108, relative to SEQ ID NO: 4. In some embodiments, the TnsB protein comprises an amino acid sequence having amino acid substitutions at positions: 4, 23 and 590; 19, 169, and 549; 43 and 415; 80 and 593; 80, 144, 593, and 606; 1, 42, 80, 593, and 606; 42, 80, 593, and 606; 156 and 604; 283, 349, and 365; 283, 349, 365, 396, and 594; 283, 349, 365, 396, 594, 596, and 131; 352 and 390; 390, 396, and 594; 396 and 594; 456 and 502; 464 and 502; 464 and 17; 17, 235, 464, and 596; 235, 352, 396, 456, and 606; 415, 456, and 502; 456, 502, and 549; 169, 456, 502, and 549; 80, 456, 502, 593, and 606; 1, 42, 80, 456, 502, 593, and 606; 80, 144, 456, 502, 593, and 606; 19, 169, 456, 502 and 549; 43, 415, 456, and 502; 352, 390, 396, and 594; 352, 390, and 396; 283, 349, 396, and 594; 11, 55, 120, 362, 584, 600, and 604; 43, 84, 144, 349, and 517; 164 and 165; 164 and 173; 362 and 446; 352, 390, 396, 549, and 594; 352, 390, 396, 464, 549, and 594; 43, 349, 352, 390, 396, 464, 549, and 594; 43, 349, 352, 390, 396, 464, 549, 594 and one or more positions selected from 63, 145, 174, 182, 208, 410, 427, 456, 504, and 526; 43, 352, 390, 396, 464, 549, and 594; 43, 349, 352, 390, 396, 464, 549, 594, 410, 526, 415, and 502; 43, 349, 352, 390, 396, 464, 549, 594, 410, 526, 415, 502, and 21; 43, 349, 352, 390, 396, 464, 549, 594, 410, 526, 415, 502, and 67; 43, 349, 352, 390, 396, 464, 549, 594, 410, 526, 415, 502, 21, and 67; 43, 349, 352, 390, 396, 464, 549, 594, 410, 526, 174, 208, 427, 456, and 504; 43, 349, 352, 390, 396, 464, 549, 594, 410, 526, 415, 502, and 139; 43, 349, 352, 390, 396, 464, 549, 594, 410, 526, 415, 502, 339, and 446; 43, 349, 352, 390, 396, 464, 549, 594, 410, 526, 19, 460, 569, and 596; 43, 349, 352, 390, 396, 464, 549, 594, 410, 526, 460, 586, 588, and 608; 43, 349, 352, 390, 396, 464, 549, 594, 410, 526, and 460; 352, 390, 396, 549, 586, and 594; 63, 158, 352, 390, 396, 549, 586, and 594; 164, 165, 352, 363, 390, 396, 410, 549, 586, and 594; 164, 173, 352, 390, 396, 549, 586, and 594; 83, 352, 390, 396, 549, 586, and 594; 8, 43, 174, 349, 352, 390, 396, 427, 464, 549, and 594; or 283, 349, 365, 396, 594, 596, and 131, relative to SEQ ID NO: 5.

In some embodiments, the TnsA protein comprises an amino acid sequence of SEQ ID NO: 4 and the TnsB protein comprises an amino acid sequence having amino acid substitutions at positions: 43, 349, 352, 390, 396, 464, 549, 594 and one or more positions selected from 63, 145, 174, 182, 208, 410, 427, 456, 504, and 526; 43, 349, 352, 390, 396, 464, 549, 594, 415 and 502; 43, 349, 352, 390, 396, 464, 549, 594, 415, 502 and 67; 43, 349, 352, 390, 396, 464, 549, 594, 415, 502 and 21; or 43, 349, 352, 390, 396, 464, 549, 594, 415, 502, 21 and 67, relative to SEQ ID NO: 5. In some embodiments, the TnsB protein comprises an amino acid sequence having amino acid substitutions at: 43, 349, 352, 390, 396, 464, 549, 594, and 456; 43, 349, 352, 390, 396, 464, 549, 594, 456, and 526; 43, 349, 352, 390, 396, 464, 549, 594, and 504; 43, 349, 352, 390, 396, 464, 549, 594, and 526; 43, 349, 352, 390, 396, 464, 549, 594, 410, and 526; 43, 349, 352, 390, 396, 464, 549, 594, 174, and 427; 43, 349, 352, 390, 396, 464, 549, 594, and 208; 43, 349, 352, 390, 396, 464, 549, 594, 63, 145, 182, and 526; 43, 349, 352, 390, 396, 464, 549, 594, 415 and 502; 43, 349, 352, 390, 396, 464, 549, 594, 415, 502 and 67; 43, 349, 352, 390, 396, 464, 549, 594, 415, 502 and 21; or 43, 349, 352, 390, 396, 464, 549, 594, 415, 502, 21 and 67.

In some embodiments, the TnsA protein comprises an amino acid sequence of SEQ ID NO: 4 and the TnsB protein comprises an amino acid sequence having amino acid substitutions of: F43S, Y349N or Y349D, P352T, A390V, D396N, H464R, Q549R, Q594L and one or more substitutions selected from R63G, A145S, A174S, I182R, V208M, Q410K, T427S, T456I or T456P, P504S, and V526E; F43S, Y349N or Y349D, P352T, A390V, D396N, H464R, Q549R, Q594L, A415V and T502I; F43S, Y349N or Y349D, P352T, A390V, D396N, H464R, Q549R, Q594L, A415V, T502I, and T21A; F43S, Y349N or Y349D, P352T, A390V, D396N, H464R, Q549R, Q594L, A415V, T502I, and Q67K; or F43S, Y349N or Y349D, P352T, A390V, D396N, H464R, Q549R, Q594L, A415V, T502I, T21A, and Q67K. In some embodiments, the TnsB protein comprises an amino acid sequence having amino acid substitutions of: F43S, Y349N, P352T, A390V, D396N, H464R, Q549R, Q594L, and T456I; F43S, Y349N, P352T, A390V, D396N, H464R, Q549R, Q594L, T456P, and V526E; F43S, Y349D, P352T, A390V, D396N, H464R, Q549R, Q594L, and P504S; F43S, Y349N, P352T, A390V, D396N, H464R, Q549R, Q594L, and V526E; F43S, Y349N, P352T, A390V, D396N, H464R, Q549R, Q594L, Q410K, and V526E; F43S, Y349N, P352T, A390V, D396N, H464R, Q549R, Q594L, A174S, and T427S; F43S, Y349N, P352T, A390V, D396N, H464R, Q549R, Q594L, and V208M; F43S, Y349N, P352T, A390V, D396N, H464R, Q549R, Q594L, R63G, A145S, I182T, and V526E; F43S, Y349N or Y349D, P352T, A390V, D396N, H464R, Q549R, Q594L, A415V and T502I; F43S, Y349N or Y349D, P352T, A390V, D396N, H464R, Q549R, Q594L, A415V, T502I, and T21A; F43S, Y349N or Y349D, P352T, A390V, D396N, H464R, Q549R, Q594L, A415V, T502I, and Q67K; or F43S, Y349N or Y349D, P352T, A390V, D396N, H464R, Q549R, Q594L, A415V, T502I, T21A, and Q67K.

In some embodiments, the TnsA protein comprises an amino acid sequence having an amino acid substitutions at position: 182, relative to SEQ ID NO: 4, and the TnsB protein comprises an amino acid sequence having amino acid substitutions at positions: 352, 390, 396, 594, and 596, relative to SEQ ID NO: 5; the TnsA protein comprises an amino acid sequence having amino acid substitutions at positions: 88, 147, and 177, relative to SEQ ID NO: 4, and the TnsB protein comprises an amino acid sequence having amino acid substitutions at positions: 352, 390, 396, 549, and 594, relative to SEQ ID NO: 5; the TnsA protein comprises an amino acid sequence having amino acid substitutions at positions: 88 and 147, relative to SEQ ID NO: 4, and the TnsB protein comprises an amino acid sequence having amino acid substitutions at positions: 352, 390, 396, 464, 549, and 594, relative to SEQ ID NO: 5; the TnsA protein comprises an amino acid sequence having amino acid substitutions at positions: 88, 116 and 147, relative to SEQ ID NO: 4, and the TnsB protein comprises an amino acid sequence having amino acid substitutions at positions: 289, 352, 390, 396, 549, 594, and 596, relative to SEQ ID NO: 5; the TnsA protein comprises an amino acid sequence having an amino acid substitution at position: 75, relative to SEQ ID NO: 4, and the TnsB protein comprises an amino acid sequence having amino acid substitutions at positions: 235, 352, 390, 396, 567, and 594, relative to SEQ ID NO: 5; the TnsA protein comprises an amino acid sequence having amino acid substitutions at positions: 88, 147, 170, and 182, relative to SEQ ID NO: 4, and the TnsB protein comprises an amino acid sequence having amino acid substitutions at positions: 352, 363, 390, 396, 549, 586, and 594, relative to SEQ ID NO: 5; the TnsA protein comprises an amino acid sequence having amino acid substitutions at positions: 88, 147, 170, 182, and 51 or 180, relative to SEQ ID NO: 4, and the TnsB protein comprises an amino acid sequence having amino acid substitutions at positions: 43, 349, 352, 390, 396, 410, 464, 526, 549, and 594, relative to SEQ ID NO: 5; the TnsA protein comprises an amino acid sequence having amino acid substitutions at positions: 75, 88, and 147, relative to SEQ ID NO: 4, the TnsB protein comprises an amino acid sequence having amino acid substitutions at positions: 352, 390, 396, 549, and 594, relative to SEQ ID NO: 5; or the TnsA protein comprises an amino acid sequence having amino acid substitutions at positions: 88, 93, and 147, relative to SEQ ID NO: 4, and the TnsB protein comprises an amino acid sequence having amino acid substitutions at positions: 352, 390, 396, 549, 580, and 594, relative to SEQ ID NO: 5.

In some embodiments, the TnsA protein comprises an amino acid sequence having amino acid substitution: F182L, relative to SEQ ID NO: 4, and the TnsB protein comprises an amino acid sequence having amino acid substitutions: P352T, A390V, D396N, Q594L, and H596Y, relative to SEQ ID NO: 5; the TnsA protein comprises an amino acid sequence having amino acid substitutions: P88T, I147V, and T177I, relative to SEQ ID NO: 4, and the TnsB protein comprises an amino acid sequence having amino acid substitutions: P352S, A390V, D396N, Q549R, and Q594L, relative to SEQ ID NO: 5; the TnsA protein comprises an amino acid sequence having amino acid substitutions: P88T and I147V, relative to SEQ ID NO: 4, and the TnsB protein comprises an amino acid sequence having amino acid substitutions: P352T, A390V, D396N, H464R, Q549R, and Q594L, relative to SEQ ID NO: 5; the TnsA protein comprises an amino acid sequence having amino acid substitutions: P88T, V116I and I147V, relative to SEQ ID NO: 4, and the TnsB protein comprises an amino acid sequence having amino acid substitutions: Q289H, P352T, A390V, D396N, Q549R, Q594L, and H596Y, relative to SEQ ID NO: 5; the TnsA protein comprises an amino acid sequence having amino acid substitution: S75I, relative to SEQ ID NO: 4, and the TnsB protein comprises an amino acid sequence having amino acid substitutions: I235T, P352T, A390V, D396N, K567R, and Q594L, relative to SEQ ID NO: 5; the TnsA protein comprises an amino acid sequence having amino acid substitutions: P88T, I147V, V170L, and F182L, relative to SEQ ID NO: 4, and the TnsB protein comprises an amino acid sequence having amino acid substitutions: P352T, L363P, A390V, D396N, Q549R, S586A, and Q594L, relative to SEQ ID NO: 5; the TnsA protein comprises an amino acid sequence having amino acid substitutions at positions: P88T, I147V, V170L, F182L, and G51V or F180L, relative to SEQ ID NO: 4, and the TnsB protein comprises an amino acid sequence having amino acid substitutions at positions: F43S, Y349N, P352T, A390V, D396N, Q410K, H464R, V526E, Q549R, and Q594L, relative to SEQ ID NO: 5; the TnsA protein comprises an amino acid sequence having amino acid substitutions: S75I, P88T, and I147V, relative to SEQ ID NO: 4, and the TnsB protein comprises an amino acid sequence having amino acid substitutions: P352T, A390V, D396N, Q549R, and Q594L, relative to SEQ ID NO: 5; or the TnsA protein comprises an amino acid sequence having amino acid substitutions: P88T, A93T, and I147V, relative to SEQ ID NO: 4, and the TnsB protein comprises an amino acid sequence having amino acid substitutions: P352T, A390V, D396N, Q549R, T580I, and Q594L, relative to SEQ ID NO: 5.

In some embodiments, the system further comprises a TnsC protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 6 with one or more amino acid substitutions, deletions, or additions relative to SEQ ID NO: 6. In some embodiments, the TnsC protein comprises an amino acid sequence having one or more amino acid substitutions at positions: 1, 2, 3, 5, 6, 7, 9, 11, 12, 14, 21, 22, 26, 27, 31, 35, 38, 43, 44, 46, 47, 54, 59, 60, 61, 64, 65, 67, 68, 71, 72, 74, 76, 79, 80, 81, 84, 89, 95, 102, 105, 109, 110, 111, 112, 113, 114, 116, 118, 119, 120, 123, 129, 130, 131, 132, 134, 142, 145, 146, 147, 148, 150, 154, 155, 166, 169, 178, 180, 181, 183, 184, 187, 190, 194, 197, 201, 204, 207, 209, 213, 219, 221, 225, 226, 227, 229, 232, 233, 234, 236, 238, 241, 246, 251, 252, 256, 257, 261, 263, 265, 267, 269, 271, 272, 274, 280, 281, 285, 286, 288, 291, 292, 296, 299, 301, 303, 304, 306, 307, 308, 310, 313, 314, 316, 317, 318, 319, 320, 323, 324, 326, 328, 330, 331, 332, 340, 341, 343, 344, 355, 412, 418, 427, 514, 1198, 1201, 1206, 1212, 1260, and 1282, relative to SEQ ID NO: 6. In some embodiments, the TnsC protein comprises an amino acid sequence having one or more amino acid substitutions of: M1L, M1V, N2S, A3T, T5P, T5A, T5S, E6D, 17S, 17V, 19F, Q11R, L12M, N14D, N14S, M21I, H22P, H22Y, K26N, K26R, T27I, M31I, L35R, N38S, S43P, D44N, D44G, Q46L, C47S, T54I, S59T, H60Y, T61A, H64Y, Y65H, K67N, K67R, R68Q, A71G, T72A, N74D, S76C, S76Y, T79I, M80I, P81S, V84L, R89L, A95D, A95T, A102T, E105D, E105K, S109N, S109R, S110P, Q111R, I112T, K113N, K113E, K114N, K114M, K114E, G116D, K118N, K118R, T119I, D120V, K123N, L129M, I130V, K131R, A132S, K134M, K134N, F142V, L145M, I146T, E147K, F148S, S150F, R154K, Q155H, E166D, K169E, P178S, A180V, A181T, A181S, I183V, A184S, A184T, A184V, P187S, A190T, A190V, V194M, V194A, R197I, Y201N, L204M, D207N, K209N, Q213H, Q213V, A219S, K221N, D225N, V226E, P227T, K229E, S232N, K233N, K233R, N234H, T236A, A238V, A238S, A241S, E246D, K251N, H252Y, H252R, E256D, A257S, A261V, S263I, S263N, N265D, Y267C, E269K, E269D, K271E, K271R, H272Y, I274V, F280L, D281N, D281G, K285G, K286N, K288R, S291F, S291P, K292N, K296R, K296N, I299S, D301G, E303D, I304T, I304V, E306G, V307L, V307G, V307A, V307D, V307G, I308N, N310S, Y313H, N314K, N316K, N316D, A317D, L318Q, D319N, P320S, P320L, M323I, L324M, D326N, V328M, V328A, A330D, I331V, V332G, S340L, T341A, A343G, S344N, I355V, F412V, V418F, Y427C, R514K, S1198L, A1201V, G1206S, C1212G, F1260L, and V1282M, relative to SEQ ID NO: 6. In some embodiments, the TnsC protein comprises an amino acid sequence having amino acid substitutions at positions: 2, 67, 95, and 226; 6 and 316; 38, 95, 303; 67, 95, and 226; 44 and 76; 44, 76, and 118; 130, 234, 303; 118 and 1201; 118, 1201, and 44; 118, 1201, and 76; 130, 234, and 303; 154 and 269; 221 and 44; 44, 76, 130, 234, and 303; 44, 76, 118, and 1201; 197 and 314; 76, 181, and 194; 76, 118, 252, and 292; 76 and 274; 76, 102, 118, and 307; 12 and 76; 67, 95, and 226; 26 and 76; 22, 76, 319; 154 and 269; 76 and 238; 76, 238, 296, and 328; 7 and 76; 76 and 263; 59, 76, 306, and 316; or 280 and 340, relative to SEQ ID NO: 6.

In some embodiments, the TnsA protein comprises an amino acid sequence having amino acid substitutions at positions: 88 and 147, relative to SEQ ID NO: 4, the TnsB protein comprises an amino acid sequence having amino acid substitutions at positions: 352, 390, 396, 464, 549, and 594, relative to SEQ ID NO: 5, and the TnsC protein comprises an amino acid sequence having amino acid substitutions at positions: 197, 314, and optionally one of 7, 12, or 114; 76 and 7, 12 or 263; or 76, 238, 296, or 328, relative to SEQ ID NO: 6; the TnsA protein comprises an amino acid sequence having amino acid substitutions at positions: 88 and 147, relative to SEQ ID NO: 4, the TnsB protein comprises an amino acid sequence having amino acid substitutions at positions: 352, 390, 396, 464, 549, and 594, relative to SEQ ID NO: 5, and the TnsC protein comprises an amino acid sequence having amino acid substitutions at positions: 76, 181, and 194, relative to SEQ ID NO: 6; the TnsA protein comprises an amino acid sequence having amino acid substitutions at positions: 88, 147, 170, and 182, relative to SEQ ID NO: 4, the TnsB protein comprises an amino acid sequence having amino acid substitutions at positions: 352, 363, 390, 396, 549, 586, and 594, relative to SEQ ID NO: 5, and the TnsC protein comprises an amino acid sequence having amino acid substitutions at positions: 197, 314, and optionally one of 7, 12, or 114; 76 and 7, 12 or 263; or 76, 238, 296, or 328, relative to SEQ ID NO: 6; the TnsA protein comprises an amino acid sequence having amino acid substitutions at positions: 88, 147, 170, and 182, relative to SEQ ID NO: 4, the TnsB protein comprises an amino acid sequence having amino acid substitutions at positions: 352, 363, 390, 396, 549, 586, and 594, relative to SEQ ID NO: 5, and the TnsC protein comprises an amino acid sequence having amino acid substitutions at positions: 76, 181, and 194, relative to SEQ ID NO: 6; the TnsA protein comprises an amino acid sequence having amino acid substitutions at positions: 88, 147, 170, 180, and 182, relative to SEQ ID NO: 4, the TnsB protein comprises an amino acid sequence having amino acid substitutions at positions: 43, 349, 352, 390, 396, 410, 464, 526, 549, and 594, relative to SEQ ID NO: 5, and the TnsC protein comprises an amino acid sequence having amino acid substitutions at positions: 197 and 314, relative to SEQ ID NO: 6; or the TnsA protein comprises an amino acid sequence of SEQ ID NO: 4, the TnsB protein comprises an amino acid sequence having amino acid substitutions positions: 43, 349, 352, 390, 396, 464, 549, 594 and one or more positions selected from 63, 145, 174, 182, 208, 410, 427, 456, 504, and 526; 43, 349, 352, 390, 396, 464, 549, 594, 415 and 502; 43, 349, 352, 390, 396, 464, 549, 594, 415, 502 and 67; 43, 349, 352, 390, 396, 464, 549, 594, 415, 502 and 21; or 43, 349, 352, 390, 396, 464, 549, 594, 415, 502, 21 and 67, relative to SEQ ID NO: 5, and the TnsC protein comprises an amino acid sequence having amino acid substitutions at positions: 197, 314, and optionally one of 7, 12, or 114; 76 and 7, 12 or 263; or 76, 238, 296, or 328, relative to SEQ ID NO: 6.

In some embodiments, the TnsA protein comprises an amino acid sequence amino acid substitutions of: P88T and 1147V, relative to SEQ ID NO: 4, the TnsB protein comprises an amino acid sequence amino acid substitutions of: P352T, A390V, D396N, H464R, Q549R, and Q594L, relative to SEQ ID NO: 5, and the TnsC protein comprises an amino acid sequence amino acid substitutions of: R197I, N314K, and optionally one of I7S, L12M, or K114M, relative to SEQ ID NO: 6; the TnsA protein comprises an amino acid sequence amino acid substitutions of: P88T and I147V, relative to SEQ ID NO: 4, the TnsB protein comprises an amino acid sequence amino acid substitutions of: P352T, A390V, D396N, H464R, Q549R, and Q594L, relative to SEQ ID NO: 5, and the TnsC protein comprises an amino acid sequence amino acid substitutions of: S76Y, A181S, and V194M, relative to SEQ ID NO: 6; the TnsA protein comprises an amino acid sequence amino acid substitutions of: 88, 147, 170, and 182, relative to SEQ ID NO: 4, the TnsB protein comprises an amino acid sequence amino acid substitutions of: P352T, L363P, A390V, D396N, Q549R, S586A, and Q594L, relative to SEQ ID NO: 5, and the TnsC protein comprises an amino acid sequence amino acid substitutions of: R197I, N314K, and optionally one of I7S, L12M, or K114M, relative to SEQ ID NO: 6; the TnsA protein comprises an amino acid sequence amino acid substitutions of: 88, 147, 170, and 182, relative to SEQ ID NO: 4, the TnsB protein comprises an amino acid sequence amino acid substitutions of: P352T, L363P, A390V, D396N, Q549R, S586A, and Q594L, relative to SEQ ID NO: 5, and the TnsC protein comprises an amino acid sequence amino acid substitutions of: S76Y, A181S, and V194M, relative to SEQ ID NO: 6; the TnsA protein comprises an amino acid sequence amino acid substitutions of: P88T, I147V, V170L, F180L, and F182L, relative to SEQ ID NO: 4, TnsB protein comprises an amino acid sequence amino acid substitutions of: F43S, Y349N, P352T, A390V, D396N, Q410K, H464R, V526E, Q549R, and Q594L, relative to SEQ ID NO: 5, the TnsC protein comprises an amino acid sequence amino acid substitutions of: R1971 and N314K, relative to SEQ ID NO: 6; or the TnsA protein comprises an amino acid sequence of SEQ ID NO: 4, the TnsB protein comprises an amino acid sequence having amino acid substitutions of: F43S, Y349N or Y349D, P352T, A390V, D396N, H464R, Q549R, Q594L and one or more substitutions selected from R63G, A145S, A174S, I182R, V208M, Q410K, T427S, T456I or T456P, P504S, and V526E; F43S, Y349N or Y349D, P352T, A390V, D396N, H464R, Q549R, Q594L, A415V and T502I; F43S, Y349N or Y349D, P352T, A390V, D396N, H464R, Q549R, Q594L, A415V, T502I, and T21A; F43S, Y349N or Y349D, P352T, A390V, D396N, H464R, Q549R, Q594L, A415V, T502I, and Q67K; or F43S, Y349N or Y349D, P352T, A390V, D396N, H464R, Q549R, Q594L, A415V, T502I, T21A, and Q67K, relative to SEQ ID NO: 5, and the TnsC protein comprises an amino acid sequence amino acid substitutions of: R197I, N314K, and optionally one of I7S, L12M, or K114M; S76Y and I7V, L12M or S263N; or S76Y, A238S, K296N, or V328M, relative to SEQ ID NO: 6.

In some embodiments, the TnsA protein comprises an amino acid sequence having substitutions at positions: 88 and 147, relative to SEQ ID NO: 4; the TnsB protein comprises an amino acid sequence having substitutions at positions: 352, 390, 396, 464, 549, and 594, relative to SEQ ID NO: 5; the TnsC protein comprises an amino acid sequence having substitutions at positions: 197, 314, and optionally one of 7, 12, or 114; 76 and 7, 12 or 263; or 76, 238, 296, or 328, relative to SEQ ID NO: 6; the Cas7 protein comprises an amino acid sequence having amino acid substitutions at position: 345, relative to SEQ ID NO: 13; and/or the Cas8-Cas5 fusion protein comprises an amino acid sequence having amino acid substitutions at position: 198, relative to SEQ ID NO: 12.

In some embodiments, the TnsA protein comprises an amino acid sequence having substitutions: P88T and I147V, relative to SEQ ID NO: 4; the TnsB protein comprises an amino acid sequence having substitutions: P352T, A390V, D396N, H464R, Q549R, and Q594L, relative to SEQ ID NO: 5; the TnsC protein comprises an amino acid sequence having substitutions: R197I, N314K, and optionally one of I7S, L12M, or K114M, relative to SEQ ID NO: 6; the Cas7 protein comprises an amino acid sequence having amino acid substitution A345R, relative to SEQ ID NO: 13; and the Cas8-Cas5 fusion protein comprises an amino acid sequence having amino acid substitution: R198H, relative to SEQ ID NO: 12.

In some embodiments, the one or more Cas proteins are encoded by a single nucleic acid. In some embodiments, the one or more transposon-associated proteins are encoded by a single nucleic acid. In some embodiments, the one or more Cas proteins and the one or more transposon-associated proteins are encoded on a single nucleic acid. In some embodiments, the one or more Cas proteins and the one or more transposon-associated proteins are encoded by different nucleic acids. In some embodiments, the one or more nucleic acids comprises one or more messenger RNAs, one or more vectors, or a combination thereof.

In some embodiments, at least one of the one or more Cas proteins and the one or more transposon-associated proteins comprises a nuclear localization signal (NLS).

In some embodiments, the TnsA and TnsB are linked in a TnsA-TnsB fusion protein. In some embodiments, the TnsA-TnsB fusion protein further comprises an amino acid linker between TnsA and TnsB. In some embodiments, the linker is a flexible linker. In some embodiments, the linker comprises a NLS.

In some embodiments, the one or more Cas proteins comprises a Cas8-Cas5 fusion protein.

In some embodiments, one or more of the at least one Cas protein and the at least one transposon-associated protein are part of a single fusion protein. In some embodiments, each of the at least one Cas protein and the at least one transposon-associated protein are part of a single fusion protein.

In some embodiments, the system further comprises at least one guide RNA (gRNA) complementary to at least a portion of a target nucleic acid, or at least one nucleic acid encoding thereof. In some embodiments, the one or more Cas protein, the one or more transposon-associated protein, and the at least one gRNA are encoded by different nucleic acids. In some embodiments, at least one of the one or more Cas protein and the one or more transposon-associated protein, and the at least one gRNA are encoded by a single nucleic acid.

In some embodiments, the at least one gRNA is a non-naturally occurring gRNA. In some embodiments, the at least one gRNA is encoded in a CRISPR RNA (crRNA) array. In some embodiments, at least one of the one or more Cas protein is part of a ribonucleoprotein complex with the at least one gRNA.

In some embodiments, the system further comprises at least one unfoldase protein, or a nucleic acid encoding thereof. In some embodiments, the at least one unfoldase protein comprises ClpX.

In some embodiments, the system further comprises a donor nucleic acid, wherein the donor nucleic acid comprises a cargo nucleic acid sequence flanked by at least one transposon end sequence. In some embodiments, the system further comprises a target nucleic acid. In some embodiments, the system is a cell-free system.

Also provided are compositions and cells comprising the disclosed systems. In some embodiments, the cell is a prokaryotic cell. In some embodiments, the cell is a eukaryotic cell (e.g., a mammalian cell, a human cell).

Additionally provided are methods for nucleic acid modification and integration. In some embodiments, the methods comprise contacting a target nucleic acid with a system, composition, or polypeptide disclosed herein.

In some embodiments, the target nucleic acid sequence is in a cell. In some embodiments, contacting a target nucleic acid sequence comprises introducing the system into the cell. In some embodiments, the cell is a prokaryotic cell. In some embodiments, the cell is a eukaryotic cell (e.g., a mammalian cell, a human cell).

In some embodiments, introducing the system into the cell comprises administering the system to a subject. In some embodiments, administering comprises in vivo administration. In some embodiments, the administering comprises transplantation of ex vivo treated cells comprising the system. In some embodiments, the system, composition, or polypeptide(s) is provided in one or more delivery vehicles. In some embodiments, the delivery vehicle one or more are selected from the group consisting of: a viral particle, a virus-like particle, a liposome, a nanoparticle, and combinations thereof.

Another aspect provided by the present disclosure is methods for generating and analyzing variant Clustered Regularly Interspaced Short Palindromic Repeats (CRISPR)-associated transposon (CRISPR-Tn) polypeptides.

In some embodiments, the methods comprise a) exposing nucleic acid sequences encoding two or more different CRISPR-Tn polypeptides to mutagenesis conditions; b) encoding one or more of TnsA, TnsB, and TnsC polypeptides on a selection phage; c) encoding crRNA, TniQ, Cas8-Cas5 fusion, Cas7, Cas6 and any of the TnsA, TnsB, and TnsC polypeptides not included on the selection phage on one or more complementary plasmids; d) encoding a phage coat protein on an accessory plasmid; and e) introducing the selection phage, complementary plasmid, and accessory plasmid to a host cell; and f) screening one or more variant CRISPR-Tn polypeptides expressed by said host.

In some embodiments, the crRNA, TniQ, Cas8-Cas5 fusion, Cas7, and Cas6 are encoded on a single complementary plasmid. In some embodiments, the crRNA is encoded on a first complementary plasmid, and TniQ, Cas8-Cas5 fusion, Cas7, and Cas6 are encoded on a second complementary plasmid.

In some embodiments, the first complementary plasmid further encodes a ribosomal binding site (RBS), a crRNA target, and a T7 RNA polymerase (RNAP) downstream of said crRNA target and RBS. In some embodiments, the first complementary plasmid further encodes an N-terminal gIII fragment linked to a Npu intein (gIII_{N}-Npu) downstream of a T7 promoter. In some embodiments, the phage coat protein is gene III (gIII) and said accessory plasmid comprises C-terminal gIII fragment linked to a Npu intein encoded downstream of a crRNA target and RBS. In some embodiments, the second complementary plasmid further comprises a donor cassette.

In some embodiments, the first complementary plasmid further encodes a ribosomal binding site (RBS) and a crRNA target. In some embodiments, the first complementary plasmid further encodes an N-terminal gIII fragment linked to a Npu intein (gIII_{N}-Npu). In some embodiments, the phage coat protein is gene III (gIII), and said accessory plasmid comprises C-terminal gIII fragment linked to a Npu intein encoded downstream of a crRNA target and RBS. In some embodiments, the second complementary plasmid further comprises a donor cassette. In some embodiments, a second complementary plasmid comprises a donor cassette.

In some embodiments, the methods comprise: a) exposing nucleic acid sequences encoding two or more different CRISPR-Tn polypeptides to mutagenesis conditions; b) encoding one or more of Cas6, Cas7, Cas8-Cas5 fusion, and TniQ polypeptides on a selection phage; c) encoding crRNA, TnsA, TnsB, TnsC and any of the Cas6, Cas7, Cas8-Cas5, and TniQ polypeptides not included on the selection phage on one or more complementary plasmids; d) encoding a phage coat protein on an accessory plasmid; e) introducing the selection phage, complementary plasmid, and accessory plasmid to a host cell; and f) screening one or more variant CRISPR-Tn polypeptides expressed by said host.

In some embodiments, the crRNA, TnsA, TnsB, and TnsC are encoded on a single complementary plasmid. In some embodiments, the accessory plasmid encodes a C-terminal phage coat protein fragment linked to an intein. In some embodiments, the complementary plasmid further encodes an N-terminal phage coat protein fragment linked to an intein downstream of a T7 RNA polymerase (RNAP). In some embodiments, the crRNA is encoded on a plasmid donor (PD).

In some embodiments, a plasmid donor comprises a donor cassette.

In some embodiments, a ribosomal binding site (RBS) is encoded on the accessory plasmid or the accessory plasmid and the complementary plasmid.

Also provided are methods for treating a disease or disorder in a subject comprising administering to the subject in need thereof a polypeptide, system or composition, or a cell comprising thereof. In some embodiments, the subject is human. In some embodiments, the system or composition comprises a donor nucleic acid encoding a therapeutic gene product or a wild-type or corrected version of a disease-associated gene.

Further provided are methods for inactivating a microbial gene, the method comprising introducing into one or more cells a system or a composition as described herein. In some embodiments, the gRNA is specific for a target site that is proximal to the microbial gene and the system or composition modifies the microbial gene. In some embodiments, the system or composition inserts a donor nucleic acid within the microbial gene. In some embodiments, the microbial gene is a bacterial antibiotic resistance gene, a virulence gene, or a metabolic gene. In some embodiments, the one or more cells are bacterial cells.

Additionally provided are methods for modifying a target nucleic acid in a plant cell comprising providing to the plant, or a plant cell, seed, fruit, plant part, or propagation material of the plant a system or a composition described herein. In some embodiments, the system or composition inserts a donor nucleic acid within the target nucleic acid. In some embodiments, the donor nucleic acid comprises a gene product.

In some embodiments, the plant is a monocot or a dicot. In some embodiments, the plant is a grain crop, a fruit crop, a forage crop, a root vegetable crop, a leafy vegetable crop, a flowering plant, a conifer, an oil crop, a plant used in phytoremediation, an industrial crop, a medicinal crop, or a laboratory model plant. In some embodiments, the system or composition is provided via Agrobacterium-mediated transformation. In some embodiments, the method confers one or more of the following traits to the plant or a plant cell, seed, fruit, plant part, or propagation material of the plant: herbicide tolerance, drought tolerance, male sterility, insect resistance, abiotic stress tolerance, modified fatty acid metabolism, modified carbohydrate metabolism, modified seed yield, modified oil percent, modified protein content, disease resistance, cold and frost tolerance, improved taste, increased germination, increased micronutrient uptake, improved flower longevity, modified fragrance, modified nutritional value, modified fruit or flower size or number, modified growth, and modified plant size.

Other aspects and embodiments of the disclosure will be apparent in light of the following detailed description.

### BRIEF DESCRIPTION OF THE DRAWINGS

FIGS. 1A-1D are exemplary vector circuit designs for phage-assisted evolution of TnsABC. In FIG. 1A, TnsA, TnsB, and TnsC are the evolving genes of interest encoded on the selection phage (SP). TnsA and TnsB are encoded in a single coding region, linked by a mammalian nuclear localization signal (NLS). This is also abbreviated as TnsAB or TnsA-bpNLS-TnsB. crRNA, TniQ, Cas8, Cas7, Cas6, and a promoter-containing donor cassette are encoded on the complementary plasmid (CP). crRNA target, RBS, and gene III (gIII) are encoded on the accessory plasmid (AP). INTEGRATE system (TnsA, TnsB, TnsC, TniQ, Cas8, Cas7, Cas6, and crRNA) catalyzes integration of the donor cassette downstream of crRNA target on AP, leading to gIII expression and SP propagation. In FIG. 1B, the circuit is a modified version of the circuit shown in FIG. 1A with: crRNA, TniQ, Cas8, Cas7, Cas6, and crRNA encoded on the complementary plasmid 1 (CP1) and the donor cassette is encoded on complementary plasmid 2 (CP2), also known as the plasmid donor (PD). In FIG. 1C, the circuit is a modified version of the circuit shown in FIG. 1B with: C-terminal gIII linked to the Npu intein (gIII_{C}-Npu) encoded downstream the crRNA target and RBS on the AP; N-terminal gIII linked to the Npu intein (gIII_{N}-Npu) encoded downstream the crRNA target and RBS on the CP; and donor cassette and crRNA is encoded on plasmid donor (PD). The INTEGRATE system catalyzes integration of the donor cassette downstream of crRNA target on AP AND downstream of the crRNA target on the CP, leading to expression of both halves of gIII and full-length pIII protein reconstitution. This circuit splits gIII across two plasmids, minimizing the chance of SP acquiring full-length gIII. In FIG. 1D, the circuit is a modified version of the circuit shown in FIG. 1C with: T7 RNA polymerase (RNAP) encoded downstream of the crRNA target and RBS on the CP; and N-terminal gIII linked to the Npu intein (gIII_{N}-Npu) encoded downstream a T7 promoter on the CP. Integration at the crRNA target on the CP now promotes T7 RNAP expression, which in turn drives gIII_{N}-Npu expression. This circuit increases the amount of gIII_{N}-Npu expressed per CP integration event, thereby reducing selection stringency.

FIGS. 2A and 2B shows that variants of TnsA, TnsB, TnsC from Tn6677 from initial phage-assisted non-continuous evolution (PANCE) propagation rounds (clones 1-4) propagated more efficiently on the selection circuit when programmed with a targeting crRNA, and this propagation correlated with integration of the donor at the AP as measured by qPCR.

FIG. 3 shows a schematic of a plasmid to plasmid mammalian cell editing used to assess the efficiency of evolved variants. Evolved variants were cloned into expression vectors and co-transfected with other components of the CRISPR system as necessary along with a donor transposon (pDonor Mini-Tn) and plasmid target (pTarget). Following incubation for 72 hours, cells were lysed and integrated target plasmid was measured by qPCR with a probe for integration 49 bp downstream of the target site.

FIG. 4A shows that variants of TnsA, TnsB, TnsC from Tn6677 from initial phage-assisted non-continuous evolution (PANCE) propagation rounds show increased plasmid to plasmid editing in mammalian cells. FIG. 4B shows a comparison of the variants of TnsA, TnsB, TnsC derived from Tn6677 of Vibrio cholerae with the system derived from Tn7016, a transposon encoded by Pseudoalteromonas sp. S983.

FIGS. 5A and 5B show that variants of TnsA, TnsB, TnsC from Tn7016 from initial phage-assisted continuous evolution (PACE) propagation rounds improved transposition in E. coli compared to wild-type (FIG. 5A) but did not have improved transposition efficiencies in mammalian cells (FIG. 5B). FIGS. 5C-5E shows variants of TnsA, TnsB, TnsC from Tn7016 from initial phage-assisted non-continuous evolution (PANCE) propagation had improved integration in E. coli (FIG. 5C) and plasmid and genomic targets in mammalian cells (FIGS. 5D and 5E).

FIGS. 6A-6D show that a variant from the initial round of PANCE was used in further propagations of PACE and PANCE to generate a series of variants which improve editing in mammalian cells. FIG. 6A shows those genotypes enabling the highest editing efficiencies. FIGS. 6B-6D show plasmid and genomic targets, as indicated. FIG. 6E shows the series of variants also improves editing efficiencies in bacteria.

FIG. 7 shows the editing efficiency from reversion of exemplary mutant variant at multiple genomic sites.

FIG. 8 are graphs of editing efficiencies for variants harvested at different timepoints during a single round of PACE/PANCE propagations.

FIGS. 9A and 9B are exemplary vector circuit designs for phage-assisted evolution of QCascade components Cas6, Cas7, Cas8, and TniQ. In FIG. 9A, TniQ, Cas8, Cas7, and Cas6 are the evolving genes of interest encoded on the selection phage (SP). crRNA, TnsAB, and TnsC are encoded on the complementary plasmid (CP). TnsA and TnsB are encoded in a single coding region, linked by a mammalian nuclear localization signal (NLS). This is also abbreviated as TnsAB or TnsA-bpNLS-TnsB. Donor cassette is encoded on plasmid donor (PD). crRNA target, RBS, and gene III (gIII) are encoded on the accessory plasmid (AP). The system catalyzes integration of the donor cassette downstream of crRNA target on AP, leading to gIII expression. In FIG. 9B, the circuit in FIG. 9A was modified by: TnsAB, TnsC crRNA target site, T7 RNAP, and N-terminal gIII linked to the Npu intein (gIII_{N}-Npu) were encoded on the complementary plasmid, the donor cassette and crRNA is encoded on plasmid donor (PD), and the crRNA target, RBS, and C-terminal gIII linked to the Npu intein (gIII_{C}-Npu) are encoded on the accessory plasmid (AP). The system catalyzes integration of the donor cassette downstream of crRNA target on AP AND downstream of the crRNA target on the CP, leading to expression of both halves of gIII and full-length pIII protein reconstitution.

FIGS. 10A and 10B show that TnsC can acquire mutations in evolution that inhibit mammalian activity. Evolved TnsAB were tested for editing efficiency in combination with wildtype TnsC and evolved TnsC with wildtype TnsAB for PANCE N23 and PACE P9 variants, as indicated, for plasmid (FIG. 10A) and genomic (FIG. 10B) targets. PACE P9 variants were often best when combining evolved TnsAB with wildtype TnsC. Plasmid: 15 cycles PCR 1. Genome: 25 cycles PCR 1.

FIG. 11A is a schematic of a TnsAB single integration circuit for Tns PACE circuit 4 (TnsAB evolution). The circuit has the following modifications compared to Tns circuit 3: TnsC is removed from SP and encoded on the CP; CP target site is removed (returning to single integration circuit); AP backbone size is increased (preventing gIII acquisition by SP); and pDonor contains a transposon left end that is either wildtype sequence or contains a mutated binding site (dubbed “s-IBS” for a putative bacterial host factor (Integration Host Factor) to prevent SP from evolving bacterial-specific fitness. The single integration circuit reduces selection stringency for TnsAB evolution and simplifies PACE circuit design. Removing TnsC from SP decreases accumulation of deleterious mutations for mammalian activity. FIGS. 11B and 11C show TnsAB PANCE N25 on Tns circuit 4. SP encoded P8-L5-8 or N23-P16-L1-2 TnsAB, the best performing TnsABs from previous TnsABC evolutions. Variants isolated at P13 and P25. * indicates selection-free drift passage.

FIGS. 12A-12C show that TnsAB PANCE N25-P13 variants are not significantly better than starting genotypes. The graphs show editing efficiencies at plasmid and genomic targets, as indicated. Arrows indicate starting TnsAB variants (P8-L5-8, N23-P16-L1-2) that yielded variants to the right. All TnsABs tested with P8-L5-8 TnsC, best TnsC at time of characterization.

FIGS. 13A-13C show that TnsAB PANCE N25-P25 variants demonstrate improved mammalian activity compared to input variants. The graphs show editing efficiencies at plasmid and genomic targets, as indicated. Arrows indicate starting TnsAB variants (P8-L5-8, N23-P16-L1-2) that yielded variants to the right. All variants tested with N23-P16-L1-5 TnsC, best TnsC at the time of characterization. N25 TnsAB variants represent some of the most active Tn7016 TnsABs. AAVS1 site quantified by HTS and ddPCR.

FIG. 14 shows the measurement of N25 TnsAB editing with ddPCR and HTS. The HTS strategy for measuring integration requires comparing integrated and unintegrated PCR amplicons, and thus % integration can be skewed by PCR bias. ddPCR is an established method for measuring integration without PCR bias, and values can be interpreted as a “ground truth” for % integration. The comparison between HTS and ddPCR show HTS values are on average ˜3.5-fold higher than ddPCR (top). Values normalized to starting activity are consistent across ddPCR/HTS (bottom). Most data shown in these slides is obtained by HTS, (denoted on graphs by the number of PCR cycles) which enables high-throughput characterization of relative editing efficiencies of variants. Absolute editing variants will be determined by ddPCR going forward, unless otherwise noted.

FIG. 15 shows the analysis of N25-P25 TnsABs with wildtype or s-IBS mutant transposons in mammalian cells. Editing at AAVS1 was tested with WT or IHF binding mutant (s-IBS) transposon donor. Evolution on WT or s-IBS transposon did not result in transposon-specific activity. Arrows indicate starting TnsAB variants (P8-L5-8, N23-P16-L1-2) that yielded variants to the right. All variants tested with N23-P16-L1-5 TnsC.

FIGS. 16A and 16B show PACE P11 of highly active N25-P25 TnsABs. Input SP were top 2 N25 TnsAB variants (FIG. 16A) and pooled N25 PANCE lagoons. Evolved on both WT (L1-L3) and s-IBS transposon (L4-L6) (FIG. 16B). L1/L2 bottlenecked at ˜144 h, thus sampled genotypes from 168 h and 120 h.

FIGS. 17A-17D show that PACE (P11) of mammalian-active TnsAB failed to substantially improve editing. Boxed are input N25 TnsAB variants into PACE. No PACE variant had significantly improved editing across sites. Higher selection stringency could further improve TnsAB mammalian activity.

FIGS. 18A and 18B show TnsAB PANCE N29-PANCE of clonally isolated top 8 N25 TnsAB variants and N25 PANCE lagoons. All evolutions done on s-IBS transposon, targeting AAVS1 sequence on AP (previously conducted evolutions on a target sequence not found in mammalian cells). Several lagoons acquired gIII (CAST-independent recombination), highlighted in red.

FIGS. 19A and 19B show TnsAB PACE P12 on Tns circuit 5. Tns circuit 5 (FIG. 19A) has the following modifications as compared to Tns circuit 4: installation of a ribosome binding site between TnsA and TnsB, splitting the synthetic TnsA-TnsB fusion into its native TnsA+TnsB form. TnsAB PACE often evolved stop codons within the bpNLS (splitting TnsA-TnsB into TnsA+TnsB) to improve circuit fitness. P12 PACE (FIG. 19B) evolved two best N25 TnsAB on Tns circuit 5 and evolved on 5 kb transposon (previous Tn7016 evolutions on 1 kb transposon) for increased selection stringency.

FIG. 20 shows the outline for TnsAB and TnsC evolution to identify TnsAB/TnsC combinations.

FIGS. 21A-21C show a TnsC screen with N25-P25-L5-5 TnsAB. Tested TnsC variants cloned into mammalian vector (69 total). Plasmid (FIG. 21A) and AAVS1 (FIG. 21B) editing efficiencies correlate. N14-5 TnsC (variant from first TnsABC PANCE) is preferred (FIG. 21C). The arrow in each of FIGS. 21A and 21B indicates WT TnsC.

FIGS. 22A and 22B show the ddPCR of top TnsC variants from screen. The top six TnsC variants, WT, and ΔTnsC were quantified by ddPCR in addition to HTS. Comparison of editing values: ddPCR shows ˜2.25% editing T-RL insertion 48 bp downstream of target (FIG. 22A). Comparison of WT-normalized values: ddPCR and HTS are consistent in identifying the best TnsCs for subsequent combinations of beneficial mutations (FIG. 22B). Editing efficiency of 2.25% by N25-P25-L5-5 TnsAB+N14-5 TnsC is higher editing than previously observed at AAVS1.

FIG. 23 shows TnsC genotypes sorted by efficiency. Mutations were sorted by editing relative to WT (averaged across P2P and genome): Green: >1.35-fold vs WT; Red: <1-fold vs WT. All single mutants associated with >1.35-fold editing and mutants that appeared in >1 beneficial variant into N14-5 TnsC. Twenty-nine mutations (green) were cloned.

FIGS. 24A-24D show a repeat of the TnsC screen as in 21A-21C in the presence and absence of ClpX to determine if TnsC fitness landscape changes with addition of ClpX. Transfection conditions changed from previous screen include: drug selection for transfected cells; harvest 4 days post transfection (instead of 3 days post transfection). FIGS. 24A and 24B show editing efficiencies correlate in the absence (FIG. 24A) and presence (FIG. 24B) of ClpX. FIG. 24C shows that the absence of ClpX aligns with results from the previous screen. Editing relative to WT is higher for this screen likely due to transfection condition changes. FIG. 24D shows that ClpX improves editing for almost all TnsC variants. ClpX improves intermediately active variants, but best TnsC variants without ClpX (like N14-5) lack significant improvement with ClpX.

FIGS. 25A-25F show a single mutation TnsC screen. Twenty-nine point mutations were individually cloned into N14-5 TnsC backbone and tested at AAVS1 (FIGS. 25A and 25C) and HEK3 (FIGS. 25B and 25D), in the presence and absence of ClpX, as indicated. Line in FIGS. 25C and 25D indicates N14-5 activity. At AAVS1, activity with and without ClpX generally correlates. At HEK3, some improvements were seen without ClpX but no improvements were seen in the presence of ClpX indicating that the advantage from TnsC mutations may be redundant with addition of ClpX. FIGS. 25E and 25F show a summary of the single mutation TnsC screen. Single mutations in N14-5 TnsC only show significant improvement at HEK3 without ClpX (which had lowest starting editing). No single mutations markedly improve editing with ClpX. Stacking of multiple mutations may be used to further improve activity. The best single mutations in N14-5 TnsC are indicated in the upper right quadrant of FIG. 25E.

FIG. 26 shows ClpX titration with and without a puromycin selection. ClpX was titrated with WT TnsABC (pink), P8-L5-8 (purple), and N25-P25-L5-5 TnsAB+N23-P16-L1-5 TnsC (blue). Toxicity was observed with high amounts of ClpX. Puromycin selection was tested to see if selection for transfected cells mitigates low editing at high ClpX doses. Puromycin selection for transfected cells did not substantially alter trends for plasmid editing, but may enable higher ClpX concentrations for genome editing. High amounts of ClpX could lead to TnsB degradation prior to transposition, or could stress cells and lower transgene expression, either of which would lower editing.

FIG. 27 shows the analysis of a representative suite of evolved TnsABCs, encompassing previous successes (N14-1, P8-L5-8, and N25 variants) and previous failures (P9-144 h variants) in the presence and absence of ClpX. Addition of ClpX generally did not affect relative efficiencies of previously evolved TnsABCs and did not rescue P9-144 h variants. Fold improvement from the inclusion of ClpX is much greater for WT and weakly active evolved variants as compared to highly active evolved variants, suggesting that evolved mutation from Tns PACE could be addressing similar bottlenecks as the addition of ClpX remedies.

FIG. 28 shows the analysis of the best evolved TnsABs (x axis) with the best evolved TnsC (y axis) at a different AAVS1 from previous in the presence and absence of ClpX. These are the same trends as seen previously, where ClpX improves efficiencies of WT and less-evolved TnsABCs more than highly evolved TnsABCs. pBK17 TnsC is a combination of PACE/PANCE TnsC mutations, genotype is in TnsC screen.

FIGS. 29A-29C show the effects of transfection stoichiometries for one of the best evolved TnsABC variants in mammalian cells. Stoichiometry of plasmid components was optimized with N23-P16-L1-5 TnsABC. All non-titrated components were kept constant according to previous stoichiometry. Completed side-by-side with re-optimization of WT TnsABC at plasmid editing-opposite trends for TnsC.

FIG. 30 shows that modifying transfection stoichiometry for PACE 9 TnsABC variants did not restore mammalian activity. Representative PACE 9 144 h TnsABCs were titrated to modify the stoichiometry and assess whether activity could be restored. Each titrated variant was tested with co-evolved subunit (L3-1 TnsAB titration tested with L3-1 TnsC). No stoichiometry enabled editing greater than N23-P16-L1-5 TnsABC.

FIGS. 31A-31C show N23-P16-L1-5 TnsABC tested with larger transposons in mammalian cells. Integration of 2 cargoes per transposon size (5 kb, 10 kb) was tested at plasmid and genomic targets, as indicated. Efficiency was reduced as a function of transposon size, though less of a drop-off in activity was seen for plasmid to plasmid editing.

FIG. 32 shows analysis of using a split TnsA/TnsB in mammalian cells. Tn7016 TnsAB fusion is an artificial construct inspired by a native TnsAB fusion in an orthologous CAST (see Vo, et al. Mobile DNA 2021). TnsA-bpNLS and bpNLS-TnsB for N23-P16-L1-5 TnsABC were tested. Adjusting stoichiometry of split TnsA-NLS and NLS-TnsB enabled editing to approximate TnsA-NLS-TnsB fusion efficiency (shown bottom right), but did not substantially improve mammalian activity.

FIGS. 33A and 33B show a comparison of TnsAB and TnsC backbones in the presence and absence of ClpX. Sternberg and Liu constructs use different mammalian expression backbones for TnsAB and TnsC: Sternberg backbones have SV40 ori, and Sternberg TnsC backbone has a consensus Kozak sequence for TnsC. All 4 combinations of Liu/Sternberg TnsAB/TnsC backbones were tested for WT and current best TnsABC, with and without ClpX. Sternberg backbones enabled optimal editing with or without ClpX. Sternberg TnsC backbone significantly improved editing efficiency for WT TnsC. WT TnsC was better than evolved TnsCs in Sternberg backbone. The difference was likely caused by different stoichiometries caused by SV40 ori as transfected cells can replicate TnsAB and TnsC vectors.

FIGS. 34A-34F show that the evolution of Tn6677 QCascade complex on circuit 1.0 leads to improved plasmid to plasmid integration efficiency in bacterial cells. FIG. 34A is a schematic of the PACE circuit 1.0 adapted from TnsABC circuit. FIG. 34B shows the overnight propagation and Tn integration with WT and evolved TnsABC. FIG. 34C shows the phage titer and lagoon flow rate over time for Tn6677 PACE 1. FIG. 34D is a schematic of the bacterial plasmid to plasmid integration assay. FIG. 34E is a table of select mutations from PACE 1. FIG. 34F is the results of the E. coli plasmid to plasmid integration for the select clones.

FIGS. 35A-35E show that the evolution of Tn7016 QCascade complex on circuit 1.0 leads to improved plasmid to plasmid integration efficiency in bacterial cells. FIG. 35A is a schematic of the PACE circuit 1.0 adapted from TnsABC circuit. FIG. 35B shows the overnight propagation and Tn integration for the indicated conditions. FIG. 35C shows the phage titer and lagoon flow rate over time for Tn7016 PANCE. FIGS. 35D and 35E show overnight propagation (left), PACE (center) and the results of the E. coli plasmid to plasmid integration (right) for the select clones with P2-L3-2 TnsABC (FIG. 35D) or N14-1 TnsABC (FIG. 35E).

FIGS. 36A-36C show Tn7016 QCascade variants have improved E. coli genomic integration efficiency (FIG. 36A) and improved plasmid editing (P2P) in mammalian cells (FIG. 36B) but reduced mammalian genomic integration efficiency measured at HEK3-2 (FIG. 36C).

FIGS. 37A-37E show construction of circuit 2.0 for the evolution of the Tn7016 QCascade complex. FIG. 37A is a schematic showing the changes from PACE circuit 1.0 to PACE circuit 2.0 single integration. FIG. 37B shows cartoons of the evolution of different PAM preferences. FIG. 37C shows that the CRISPR repeat affects integration efficiency. FIG. 37D shows integration with an improved TnsABC variant (N20/P8). FIG. 37E shows the toxicity of TnsABC variants in bacterial cells.

FIGS. 38A and 38B show that evolution on circuit 2.0 is possible with PANCE and regular monitoring for cheater phage. Cheating lagoons were discontinued and new lagoons were seeded with phage from either one of the non-cheater lagoon or a pool of phage from non-cheater lagoons (FIG. 38A). Phage propagation increased but there was a reduced number of distinct genotypes. There were five failed PACE attempts on circuit 2.0 (FIG. 38B).

FIGS. 39A and 39B show that evolution campaigns on circuit 2.0 led to new, heavily mutated QCascade variants with ˜0% integration efficiency in HEK293T cells at both a genomic site (FIG. 39A) and plasmid to plasmid transfer (FIG. 39B). HTS done at high PCR 1 cycle count: values likely skewed from PCR bias.

FIG. 40 shows the integration at a genomic site with evolved QCascade components individually with wildtype counterparts. HTS done at high PCR 1 cycle count: values likely skewed from PCR bias.

FIG. 41 is a schematic showing the evolution of circuit 4.0 which enables cheater-free evolution of Tn7016 QCascade complex.

FIGS. 42A and 42B show that phage propagate (FIG. 42A) and integrate (FIG. 42B) more efficiently on circuit 4.0 compared to previous circuits.

FIGS. 43A and 43B show the results of the circuit 4.0 (v4) evolved variants. None of the v4-evolved variants show consistently higher integration efficiency across multiple sites. FIG. 43A shows the integration efficiency measured by HTS for AAVS1, HEK3-2 (25 cycles PCR1) and P2P (15 cycles PCR1). Evolved QCascade variants from circuit v4 are shown by variant name (4V1-4V8). WT combinations include variant name-evolved component. Editing efficiencies are shown as fold improvement over WT QCascade. Variants from phage which did particularly well during PANCE (v4, v8) are among the variants with the lowest editing efficiency in mammalian cells. FIG. 43B shows the editing efficiencies measured by ddPCR are ˜4× lower than low-cycle HTS values but relative values are the same, thus the ddPCR data correlates well with HTS data. Potentially improved integration at AAVS1 site with 4V2 (mutations only present in Cas6), and 4V6-6. 4V6-6 may be evolved further with the single subunit evolution circuit.

FIG. 44 shows the results from using WT combinations of evolved Tn7016 QCascade components. Conditions with greater than one evolved QCascade component have among the lowest editing efficiencies motivating single subunit evolution. Improvement seen using evolved Casos in combination with WT Cas7, 8 & TniQ.

FIG. 45 shows that a combination of potentially beneficial mutations and reversion of potentially harmful mutations did not lead to increased integration efficiency. Repeat experiment with evolved Cas6 variants do not show any significant improvement at AAVS1 site (blue arrows). Conserved mutations in Cas6 hurt activity in a mammalian context (red arrows). Insignificant improvements with Cas7 mutations in the context of N23 P16 L1-5 transposase (black arrows).

FIGS. 46A-46C show that evolved QCascade variants show different trends in bacterial cells than in mammalian cells. Two biological replicates with two technical replicates each for each of 4 representative genotypes from PACE circuit v2 and v4 were monitored for integration efficiency (FIG. 46A). Integration efficiency for WT and v4V5, v4V6 lower than expected whereas v4V5, v4V6 transformed poorly. FIG. 46B shows lower integration efficiency of P8 L5-8 Tn. The potential reasons for the lower integration efficiency may include integration at crRNA cassette soaking up available transposon for integration and toxicity. Transformation into freshly prepped competent cells rescues activity of v4V5 & v4V6 but also improves WT activity (FIG. 46C).

FIG. 47 shows analysis of evolved QCascade components with evolved TnsABC in the presence and absence of ClpX (“SLF”).

FIGS. 48A-48F show transfection optimization with ClpX (“SLF”) and reevaluation of evolved QCascade variants. SLF improves integration efficiency significantly both with and without puromycin selection at 48-well plate (FIG. 48A; ˜ 42k cells per well). Low cell-density transfection (24-well plate (˜20k cells per well)) boosts integration efficiency further to ˜0.3% getting close to Sternberg lab values (˜1.0%) but most cells (˜80%) died (FIG. 48B). FIGS. 48C and 48D show results from v2 (circuit version 2), V5 (variant 5)-evolved component. In context of evolved TnsAB & C variant, only small improvements with SLF (˜1-3× depending on transfection condition). QCascade mutation A345T from variant v2V5-7 marginally better in absence of SLF but not in presence of SLF. FIGS. 48E and 48F show results from v4 (circuit version 4), V5 (variant 5)-evolved component. In context of evolved TnsAB & C variant, only small improvements with SLF (˜1-3× depending on transfection condition). Evolved QCascade variant v4V6-7 from circuit v4 marginally better than WT in both +/−SLF condition. FIGS. 48C and 48E—48-well plate (˜42k cells per well). FIGS. 48D and 48F—24-well plate (˜20k cells per well).

FIG. 49A shows that Cas7 A345T potentially increases DNA binding affinity. Red: mutations after 30 passages of PANCE on circuit 2.0 (111 mutations total). Alpha-folded Tn7016 structure mapped onto Tn6677 structure (PDB 6PIJ). FIG. 49B shows the mutation table for QCascade circuit v2.

FIGS. 50A-50C show structure-based rational engineering to improve DNA-binding affinity. FIG. 50A shows Tn6677 QCascade and Tn7016 QCascade Cas8 DNA binding residues. Subtle changes: R20K, R21K, S24Q, K88R, R93K, N134Q, R233K. Electrostatic mutations: S24K, S24R, H124R, N134R, R20E, R21E, K88E, R93E, R241E. FIG. 50B shows Tn6677 QCascade and Tn7016 QCascade Cas9 DNA binding residues. Subtle changes: Q236S, K343R, K344R. Electrostatic mutations: N5K, N5R, T47R, T71R, Q236E, N5D, T47D, T71D, K343E, K344E. FIG. 50C shows Cas7 structure-based rational engineering to improve DNA-binding affinity. All mutants tested with 20 ng ClpX. Subtle changes: Q236S, K343R, K344R. Electrostatic mutations: N5K, T47R, T71R, Q236E, T71D, K343E, K344E.

FIG. 51 shows PACE-inspired rational mutagenesis of Cas7 mutants. All mutants tested with 20 ng ClpX. Subtle changes: A345S, A345Y. Electrostatic mutations: A345R, A345K, A345D, A345E.

FIGS. 52A-52F show arginine screen of DNA-binding residues to improve DNA/crRNA-binding affinity. In FIG. 52A, DNA/crRNA-binding residues of Cas7 (red, left) and Cas8 (red, right) mutated to Arg. Tn7017 QCascade structure was predicted with alpha-fold and mapped onto Tn6677 QCascade (PDB 6PIJ). FIG. 52B shows Cas 7 arginine mutations with increased integration efficiency. All mutants tested with 20 ng ClpX. Values dependent on ddPCR machine (BioRad vs Qiagen). FIG. 52C shows that Cas7 double and triple mutants lead to further improvement in integration efficiency. dPCR (% positive partitions) vs. ddPCR (% positive droplets). Optimized quantification workflow: 100-400 ng of crude lysate loaded directly onto (d) dPCR machine. FIGS. 52D-52F show that improvements are significant in context of other TnsABC variants (P12 L2-6 TnsAB and N25 P15 L5-5 TnsAB) but do not translate to all genomic sites (FIG. 52D—AAVS1; FIG. 52E—HEK3-2; FIG. 52F—FANCF).

FIG. 53 shows rational mutagenesis of QCascade to decrease crRNA binding affinity. Top, Cas7 mutations predicted to interact with the crRNA based on alpha-folded Tn7016 structure. Bottom, none of the rationally engineered Cas7 mutations lead to higher integration efficiency. Cas8 R198H mutation obtained through PACE on circuit v4.

FIGS. 54A-54E show that beneficial arginine residues are located within flexible regions of the alpha-folded Tn7016 QCascade structure. FIG. 54A shows cluster 1 and cluster 2 from flexible internal and C-terminal regions, respectively and an additional beneficial mutation (N5R) with the structure. FIG. 54B shows stacking of arginine mutations across and within clusters. Mutations across clusters are stackable. Stacking mutations within cluster 2 reduces integration efficiency. Likely deleterious to have multiple neighboring arginine residues. FIG. 54C shows that site-dependence of rationally engineered Cas7 arginine residues due to possible more favorable interaction with guanine. FIG. 54D shows improvements at AAVS1-1 site with orthologue-inspired rational engineering. FIG. 54E is a summary of rationally engineered Cas7 variant with evolved TnsB/C variants. 1 kb transposon integration in HEK293T cells. x axis labels indicate Cas7 genotypes. n=2 for FANCF, n=4 for HEK3 and AAVS1.

FIG. 55 shows a summary of the TnsABC evolution. Extensive evolution of TnsABC following N14-1 failed to further improve mammalian integration activity (1 kb transposon integration in HEK293T cells).

FIGS. 56A-56C shows efficiency of evolved subunits in mammalian cells and TnsC mutations that inhibit mammalian integration activity. FIG. 56A is a summary of mammalian integration activity (1 kb transposon integration in HEK293T cells). FIG. 56B shows a chart of TnsC mutations identifying mutations which hinder mammalian activity. FIG. 56C shows reversion analysis of selected TnsCs (as shown in FIG. 56B) in HEK293T cells with 1 kb transposon integration. Dashed line indicates WT TnsC activity. Arrow indicates key mammalian-deleterious mutation.

FIGS. 57A-57F show PACE of Tn7016 TnsAB. FIG. 57A shows a schematic of TnsAB PACE (Tns Circuit 4/5). TnsC was moved from SP to CP in host E. coli to prevent accumulation of mammalian-deleterious mutations during evolution. FIG. 57B is a summary of PACE P12 characterization with 1 kb transposon integration in HEK293T cells. FIGS. 57C and 57D show full characterization of mammalian genomic integration (1 kb transposon integration in HEK293T cells) at two different sites, AAVS1 (FIG. 57C) and HEK3 (FIG. 57D) in the presence and absence of ClpX. FIG. 57E is a mutation table showing P12-L2-6 variant of TnsA and TnsB. FIG. 57F shows that mutations in TnsB are the main source of improvements in mammalian efficiency (1 kb transposon integration in HEK293T cells).

FIGS. 58A-58D show interrogation of ClpX influence on mammalian activity. ClpX enhances genomic integration in WT Tn7016 (FIG. 58A) but PACE reduced dependence on ClpX for mammalian activity (FIG. 58B). 1 kb transposon integration in HEK293T cells. FIG. 58C is a schematic and western blot showing the establishment of a ΔclpX host strain for CAST PACE. Deletion of endogenous clpX from PACE host strain (S2060) was accomplished using lambda Red recombineering. FIG. 58D shows that ΔclpX introduces new selection pressure for CAST PACE.

FIGS. 59A-59J show PACE of Tn7016 TnsAB and TnsB. FIG. 59A is a schematic of Tns circuit 6 for TnsB PACE. Tns circuit 5 with the following modifications: removal of tnsA from SP; and addition of tnsA to CP. Modified to focus (main evolution on TnsB source of improved mammalian integration). FIGS. 59B and 59C show PACE of Tn7016 TnsAB and TnsB in ΔclpX E. coli. FIG. 59B shows TnsAB PACE (Tns Circuit 5 on ΔclpX host). FIG. 59C shows TnsB PACE (Tns Circuit 6 on ΔclpX host). Dashed line in both FIGS. 59B and 59C indicates P12-L2-6 activity (input variant for ΔclpX evolutions). FIGS. 59D-59G show characterization of mammalian genomic integration for PANCE N30, PACE P13, PANCE N31 and PACE P14, respectively, as outlined in the schematics shown in FIGS. 59B and 59C. 1 kb transposon integration in HEK293T cells. X axis labels indicate TnsAB genotypes (FIGS. 59D and 59E) or TnsB genotypes (FIGS. 59F and 59G). FIG. 59H is a schematic of evolution leading to TnsB variants-76 passages of PANCE, 300 h of PACE≈1000 evolutionary generations.

FIG. 59I is a mutation table for TnsB of leading variants. FIG. 59J is a summary of integration activity for the leading variants shown in FIG. 59I as compared to WT. PACE has improved integration activity >150-fold without ClpX and >20-fold with ClpX.

FIGS. 60A-60C shows PACE P15 of TnsB. FIG. 60A shows a schematic of design of PACE P15. TnsA-specific PCR of P15 lagoons (FIG. 60B) indicated that all P15 lagoons (thought to be evolving TnsB SP) were contaminated with TnsAB SP (likely from PACE apparatus). Lagoons P15-L1, L2, L3 had trace contaminant (all sequenced SP were TnsB) and lagoons P15-L4, L5, L6 had ˜100% contaminant (all sequenced SP were TnsAB). Given that TnsAB contaminants outcompeted the TnsBs in P15 lagoons LA, L5, and L6, genotypes from these lagoons were tested in HEK293T cells (see FIG. 60C). PACE P15 TnsB genotypes from L1, L2, L3 were not tested due to a lack of new coding mutations acquired during PACE. FIG. 60C is a summary of PACE P15 mammalian genomic integration (1 kb transposon integration in HEK293T cells). Tested evolved TnsBs only (contaminant TnsABs lacked new consensus coding mutations in TnsA, see description of FIG. 60B). No contaminant P15 TnsB genotypes had activity that significantly exceeded P14-L4-5 TnsB. x axis labels indicate TnsB genotypes.

FIGS. 61A and 61B shows rational combinations of PACE P14 TnsB mutations. Twelve mutations from the top eight TnsB variants were individually introduced into P14-L4-5 (FIG. 61A). Yellow mutations were not tested in initial mammalian characterization. No point mutation significantly improved activity compared to P14-L4-5 across all conditions (FIG. 61B).

FIG. 62 shows the characterization of evolved TnsABCs in HeLa cells as compared to HEK293T cells. HeLa cells were transfected with lipofectamine 2000 using the same protocol as HEK293T cells using P12-L2-6 TnsB+N14-5 TnsC with all other CAST components WT.

FIGS. 63A-63K show the high stringency evolution of TnsB (Tns Circuit 6 on ΔclpX host). FIG. 63A is a schematic of the PACE evolution of TnsB. Three TnsB variants from PACE P14 were evolved under higher selection stringency by reducing strengths of the promoter encoded in transposon and the ribosome binding site (RBS) upstream gIII (FIG. 63B). PACEs P19, P21, and P22 all had severe bottlenecks in SP titer early in evolution (within 72 hours), suggesting previously evolved TnsB variants were incapable of supporting robust SP propagation under higher selection stringencies. FIG. 63C shows the P14-L4-5 TnsB on hosts of varying stringency. Parentheses indicate promoter strength-RBS strength for each host. FIG. 63D shows characterization of PACE P19 mammalian genomic integration (1 kb transposon integration in HEK293T cells; x axis labels indicate TnsB genotypes). FIG. 63E is a summary of the PACE P19 TnsB variants. Tns PACE has enabled greater than 15% integration (ddPCR) at AAVS1 and HEK3 in HEK293T cells. FIG. 63F shows phage titer and lagoon flow rate over time for PACEs P17, P19, P21, and P22. Clonal SP from PACE P19 (P19-L3-5) and P22 (P22-L1-4) have slightly improved activity-dependent overnight propagation on selection strain E. coli compared to input SP (P14-L4-5) (FIG. 63G). Evolution minimally improved SP fitness-often a greater than 1E3-fold improvement in activity-dependent propagation is observed following a successful PACE campaign, whereas here an approximate 1E1-fold improvement was observed). FIGS. 63H-63K are mutation tables for PACEs P17, P19, P21, and P22, respectively.

FIGS. 64A-64I show a summary of the characterization of evolved TnsBs with unique genotypes from PACEs P19, P21, P22 in HEK293T cells with WT TnsA, N14-5 TnsC, WT QCascade. Few TnsB variants show significantly improved activity compared to P14-L4-5 across both target sites (FIG. 64A). Dashed lines represent P14-L4-5 editing average of n=2. Dots represent TnsB variant editing average of n=2. All without ClpX. Variants that had slight improvements (in upper right quadrants of graphs) were selected for additional characterization. FIGS. 64B-64G show full characterization of PACEs P19, P21, P22 at two genomic locations in HEK293T cells. 1 kb transposon integration; WT TnsA, N14-5 TnsC, WT QCascade; x axis labels indicate TnsB genotypes. FIG. 64H shows replicates of PACE P19 TnsBs in HEK293T cells. Best PACE P19 variants are not significantly better than P14-L4-5 upon additional replicates. FIG. 64I shows replicates of PACE P22 TnsBs in HEK293T cells at four genomic locations. No variants significantly better than P14-L4-5 (indicated by dashed line) across all target sites. P14-L4-5 is the PACE-generated TnsB with the highest activity in HEK293T cells.

FIGS. 65A-65C show characterization of rational combinations of PACE P14 TnsB mutations. Single mutations installed in P14-L4-5 do not confer significantly improved integration activity across all conditions tested. FIG. 65A is a mutation table of TnsB and installed combination mutations (“5 mut” and “6 mut” of P14-L4-5). FIGS. 65B and 65C are integration efficiencies at two different genomic loci with and without ClpX. The combinations of mutations into P14-L4-5 did not significantly improve integration activity.

FIGS. 66A-66K show analysis of TnsABC combinations. The prior best performing combinations of TnsA, TnsB and TnsC components are shown in FIG. 66A. A screen was designed to analyze the activity of P14-L4-5 TnsB with previously evolved TnsAs and TnsCs by separately testing TnsAs with P14-L4-5 TnsB and N14-5 TnsC and TnsCs with WT TnsA and P14-L4-5 TnsB at two genomic locations AAVS1 and HEK3, all in the absence of ClpX. FIGS. 66B and 66C show the full characterization of evolved TnsAs with P14-L4-5 TnsB and N14-5 TnsC for a 1 kb transposon integration, WT QCascade, without ClpX. In FIG. 66B, the darkened bar is the results for WT TnsA. In FIG. 66C, dashed lines represent WT TnsA average of n=2; dots represent TnsA variant editing average of n=2; and green, dots labeled by TnsA genotype, indicate TnsAs selected for subsequent characterization. FIGS. 66D and 66E show the full characterization of evolved TnsCs with P14-L4-5 TnsB and WT TnsA for a 1 kb transposon integration, WT QCascade, without ClpX. In FIG. 66D, the darkened bar is the results for WT TnsC and the blue bar indicates N14-5 TnsC. In FIG. 66E, dashed lines represent WT TnsC average of n=2; dots represent TnsC variant editing average of n=2; and green, dots labeled by TnsC genotype, indicate TnsCs selected for subsequent characterization. FIG. 66F shows the characterization of wild-type and the three best evolved TnsAs (as indicated in legend) with wild-type and 5 best evolved TnsCs (x axis) at four genomic locations for a 1 kb transposon integration, P14-L4-5 TnsB, WT QCascade, without ClpX. FIG. 66G-66I show a summary of the TnsABC combinations in HEK293T cells. Combination of P12-L6-5 TnsA, P14-L4-5 TnsB, and N14-5 TnsC is the highest performing evoTnsABC combination tested. FIGS. 66J-66K are mutation tables for evolved TnsAs and TnsCs, respectively. Those shown in green were high performing in initial screens.

FIGS. 67A and 67B show the characterization of evolved CAST systems using P14-L4-5 TnsB and N14-5 TnsC at a variety of target sites. Preliminary data measured by HTS; ND=no data (<5000 total reads aligned in HTS). The results, when averaged across all sites show that evoCASTs improve integration activity 44-fold without ClpX and 15-fold with ClpX (based on HTS measurement) and, when averaged across best site for each locus evoCASTs improve integration activity 67-fold without ClpX and 10-fold with ClpX (based on HTS measurement) (FIG. 67B).

FIGS. 68A-68C show results from screening gRNAs across 6 locations. The initial screen was quantified by HTS (FIGS. 68A and 68B), with highest edited sites requantified via ddPCR with a genome: transposon junction probe (method outlined in Lampe, King, et al. Nature Biotechnology 2023) (FIG. 68C). All experiments were carried out with a 1 kb transposon integration, WT QCascade, WT TnsA, P14-L4-5 TnsB, and N14-5 TnsC. AAVS1-1 in this screen was previously referred to as “AAVS1.” HTS and junction ddPCR are roughly consistent for most sites, though most sites show higher HTS values than ddPCR, likely due to PCR bias for integrated amplicons.

FIGS. 69A-69D show the effect of crRNA architecture of integration efficiencies. Atypical and typical crRNA support similar integration efficiencies in E. coli for Tn7016. Previous mammalian characterization primarily used atypical crRNA architecture in mammalian cells, finding that atypical and typical crRNA have similar efficiencies for WT Tn7016 CAST in HEK293T cells. All characterization of evolved variants was done with typical crRNA, except for the screening of 44 common transgene insertion sites shown in FIG. 68 which used atypical crRNA. A comparison of typical vs. atypical crRNA architectures for best edited site(s) from target site screen, performed in HEK293T cells (FIGS. 69A and 69B). Typical crRNA outperforms atypical crRNA across all loci tested for evoCAST. Sequences for unprocessed crRNA (“pre-crRNA”): Typical Tn7016 Cascade crRNA: GTGACCTGCCGTATAGGCAGCTGAAAAT (SEQ ID NO: 22)[spacer]GTGACCTGCCGTATAGGCAGCTGAAAAT (SEQ ID NO: 22); Atypical Tn7016 Cascade crRNA: GTGACCTGCCGTATAGGCAGCTGAAGAT (SEQ ID NO: 23)[spacer] AATTCTGCCGAAAAGGCAGTGAGTAGT (SEQ ID NO: 24). Previous mammalian characterization by primarily used 33 nt spacer for crRNA in mammalian cells, finding that 33 nt spacer lengths had slightly improved activity compared to 32 nt spacer lengths for WT Tn7016 CAST in HEK293T cells (Lampe, King et al. Nature Biotechnology 2023), whereas characterization of evolved variants above was done with 32 nt spacers for crRNAs. FIGS. 69C and 69D show a comparison of 32 vs. 33 nt spacer length for the best edited site at each loci from target site screen, performed in HEK293T cells. 32 nt spacer is equivalent to or outperforms 33 nt spacer across all loci tested for evoCAST. 1 kb transposon integration; WT QCascade, WT TnsA.

FIGS. 70A-70D show effects of transfection conditions on integration efficiencies. FIGS. 70A and 70B show the effect of transfection conditions for HEK293T cells. Transfection with Lipofectamine 3000 (previously Lipofectamine 2000) and increased puromycin concentration (previously 1 μg/mL) may further increase integration efficiencies observed in HEK293T cells. FIGS. 70C and 70D show the effect of transfection conditions for HeLa cells. Transfection with Lipofectamine 3000 may also improve integration efficiencies in HeLa cells (though efficiencies with Lipofectamine 2000 are unusually low). All efficiencies measured by HTS.

FIGS. 71A and 71B show specificity characterization of evoCASTs. FIG. 71A is a schematic of UDiTaS-based detection of off-targets. FIG. 71B is UDiTaS of host E. coli (encoding WT QCascade/TnsA and N14-5 TnsC) following overnight incubation with SP encoding evoTnsB.

FIG. 72A is an overview of DNA binding circuit. FIG. 72B is a DNA binding circuit with TnsC-rpoZ fusion.

FIGS. 73A-73D show DNA-binding independent phage propagation with Cas6-rpoZ fusion. FIG. 73A is a schematic of the Lux assay 1.0. FIG. 73B is a schematic of PANCE 1.0. FIGS. 73C and 73D show the fold propagation of two hosts-evoCas78 (p6): phage pool from PANCE passage 6; neg.: TnsABC phage; dCas8 (R241A, P242A). Phage propagation is most likely independent of target DNA binding.

FIGS. 74A-74L show characterization of TniQ-rpoZ and TnsC-rpoZ fusion constructs. FIG. 74A is a schematic of Lux assay 2.0 with the following differences as compared to lux assay 1.0 as in FIG. 73A: P3 copy number changed from p15A to SC101; P2 promoter/RBS changed from J sd8 to pro1 SD8 potentially avoiding a potential hook effect; promoter on P1 changed from Pbad to pro1 enabling rpoZ-TniQ and TnsC-rpoZ fusions. The lac promoter was optimized for increased signal to noise (*) and rpoZ was mutated (****). FIG. 74B is schematics of constructs used in screening. In this second round of screening, all constructs used the SC101, pro1, SD8 backbone. The rpoZ domain was fused either to Cas6, TniQ, Cas7, or TnsC. The distance between the protospacer and lac promoter was increased in 2 bp increments to enable maximal circuit turn-on upon RNAP recruitment. For each architecture two different protospacers, AAVS1-1 and 0155, were tested. FIG. 74C shows great signal to noise with TnsC-rpoZ fusion on 0155 protospacer but not on AAVS1-1 protospacer. FIG. 74D shows signal to noise with rpoZ-TniQ fusion and 0155 spacer. Distance d: protospacer-P_{lac}*distance. T: targeting host with matching 0155 protospacer/spacer sequence. NT: nontargeting host with AAVS1-1 protospacer and 0155 spacer (TnsABC circuit spacer). FIG. 74E shows Lux expression on different space sequences with rpoZ-TniQ fusion. FIGS. 74F and 74G show phage encoding the Tn7016 Cascade complex propagate on hosts with the TnsC-rpoZ fusion; SP Cas 678 (FIG. 74F) or QCas (FIG. 74G). FIGS. 74H and 74I show that phage encoding the Tn7016 QCas78 propagate on hosts with the TniQ-rpoZ fusion. T: targeting host with matching 0155 protospacer/spacer sequence; NT: nontargeting host with AAVS1-1 protospacer and 0155 spacer. FIG. 74J shows overnight propagation of Cas7 and Cas8 on TniQ-rpoZ DNA binding circuit showing DNA binding dependent phage propagation. dCas78: Cas8 (R241A, P242A), impaired DNA unwinding capabilities (negative control). FIG. 74K shows the evolutionary trajectory of Cas7 and Cas8 on TniQ-rpoZ DNA binding circuit. FIG. 74L shows the overnight propagation of Cas7 and Cas8 on TniQ-rpoZ DNA binding circuit had improved phage propagation with evolved Cas7 and Cas8. evoCas78: phage pool after 19 passages of PANCE on 0155 spacer.

FIG. 75A shows a schematic of Cas7/8 DNA-binding circuit. DNA-binding circuit is referred to as version 5 circuit (v5). Upon successful QCascade complex assembly and target binding, RNAP is recruited through the rpoZ (ω) domain driving gIII expression and phage propagation. Evolution for improved complex assembly, target search, and binding. FIGS. 75B and 75C show improved lux signal with evolved Cas7/8 variants. Modestly improved transcriptional activation with evoCas7/8 from v5 PACE1. Increased activity on 0155 spacer correlates with AAVS1-1 spacer. Transcriptional activation of L2-2, L3-3, L3-5, and L4-3 variant significantly above background levels. FIGS. 75D and 75E show improved lux signal with evolved Cas7/8 variants including genotypes (L2-1, L2-6) containing rationally identified mutation K235R. Increased lux signal of L4-3 primarily driven by L4-3 Cas8. FIG. 75F shows improved phage propagation with evolved Cas7/8 phage: L3-3 Cas78: clonal phage; L1-L3 Cas7/8: clonal phage pools; dCas78: Cas8 (R241A, P242A). FIGS. 75G and 75H are mutation tables for evolved Cas8 and Cas7, respectively. For the characterization assays substitutions at K4 and E8 in Cas8 were restored to wild-type.

FIGS. 76A and 76B show that phage propagation/transcriptional activation does not always correlate with mammalian integration efficiency with evolved Cas7/8 variants. L4-3 (strongest transcriptional activation in bacterial cells) among the lowest integration values in mammalian cells. L3-3 (significantly improved activation in bacterial cells) and significantly improved integration.

FIG. 77 shows that evoCas7 and/or evoCas8 is responsible for a decrease/increase in integration efficiency. Improvements with L3-3 at AAVS1-1 site driven by evoCas7. Reduced integration efficiency of L4-3 caused by evoCas8. Reduced integration efficiency of L4-5 caused by evoCas7.

FIGS. 78A and 78B show that conserved mutations in isolation show significantly increased E. coli transcriptional activation (FIG. 78A) but no change in mammalian (HEK293T cells) integration (FIG. 78B).

FIGS. 79A-79D show evolved Cas7/8 variants with evoTnsABC across different target sites. L4-3 evoCas7/8: highest signal in lux assay but significantly reduced activity in mammalian cells across all target sites tested (FIG. 79A). Activity was partially rescued by WT Cas8 (FIG. 79B). L4-3 Cas8 significantly reduced integration efficiency across target sites (FIG. 79C). Small improvements in activity were seen with Cas7 L3-3 across highly edited target sites (FIG. 79D).

FIGS. 80A and 80B show the identification of new Cas7/8 variants with high-stringency evolution on sd2 RBS. FIG. 80A shows genotypes from PANCE on sd2 RBS. FIG. 80B shows genotypes from PACE on sd2 RBS. Improvements with a few variants across the three target sites tested. FIGS. 80C and 80D are mutation tables for evolved Cas8 and Cas7, respectively. For the characterization assays, substitutions at K4, E5, L6, I9, D11 and T12 in Cas8 were restored to wild-type.

FIGS. 81A-81D show reversion analysis of P14-L4-5 TnsB in HEK293T cells. FIG. 81A shows evolution of P14-L4-5. Each of ten mutations in P14-L4-5 were restored to its wild-type identity (FIG. 81B). All mutations appear to contribute modestly to the efficiency of P14-L4-5 (1 kb transposon integration; WT QCascade, WT TnsA, WT TnsC), as each revertant is approximately ˜50% the activity of P14-L4-5. Q549R and Q594L appear to contribute less to increased activity, though reversions of these mutations do not yield variants with significantly higher activity than P14-L4-5. Reversion analysis was also performed with ClpX. Absolute editing efficiencies are shown in FIG. 81C and relative integration ClpX: No ClpX is shown in FIG. 81D. WT TnsB benefits substantially from ClpX (˜5.5-fold at AAVS1, ˜30-fold at HEK3), whereas P14-L4-5 and all single revertants benefit modestly (˜1.5-fold at AAVS1 and HEK3).

FIG. 82 shows characterization of evolved Tn7016 CASTs in K562 cells conditions.

FIGS. 83A-83C show Cas8 variants in QCascade tested with evoTnsABC. FIG. 83A shows the Cas8 variants which contain mutations in two DNA-contacting interfaces of Cas8-PAM interacting domain and helical bundle. FIG. 83B shows integration efficiency at 6 different genomic locations. The x-axis labels indicate Cas8 genotypes. FIG. 83C shows a summary of fold-change in T-RL integration relative to WT QCascade. All screening was completed using evoTnsABC (P12-L6-5 TnsA, P14-L4-5 TnsB and N14-5 TnsC) as shown in FIG. 66H, without ClpX, and 1 kb transposon integration.

FIGS. 84A-84C show Cas7 variants in QCascade tested with evoTnsABC. FIG. 84A shows the Cas7 variants. FIG. 84B shows integration efficiency at 6 different genomic locations. The x-axis labels indicate Cas7 genotypes. FIG. 84C shows a summary of fold-change in T-RL integration relative to WT QCascade. All screening was completed using evoTnsABC (P12-L6-5 TnsA, P14-L4-5 TnsB and N14-5 TnsC) as shown in FIG. 66H, without ClpX, and 1 kb transposon integration.

FIGS. 85A and 85B show QCascade NLS architecture variants tested with evoTnsABC. Four different architecture variants were tested: Original architecture-1×NLS TniQ+1×NLS Cas6+1×NLS Cas7+1×NLS Cas8; NLS architecture 1-2×NLS TniQ+2×NLS Cas6+1×NLS Cas7+2×NLS Cas8; NLS architecture 2-3×NLS TniQ+2×NLS Cas6+1×NLS Cas7+3×NLS Cas8; and NLS architecture 3-3×NLS TniQ+2×NLS Cas6+1×NLS Cas7+4×NLS Cas8. FIG. 85A shows integration efficiency at 6 different genomic locations. The x-axis labels indicate NLS architectures. FIG. 85B shows a summary of fold-change in T-RL integration relative to original architecture. All screening was completed using evoTnsABC (P12-L6-5 TnsA, P14-L4-5 TnsB and N14-5 TnsC) as shown in FIG. 66H, WT QCascade, without ClpX, and 1 kb transposon integration.

FIG. 86 shows the screening guideRNAs targeting therapeutically relevant human genomic loci. Forty targets across eight therapeutically relevant loci (five sites per locus) were screened by HTS using evoTnsABC (P12-L6-5 TnsA, P14-L4-5 TnsB and N14-5 TnsC) as shown in FIG. 66H, WT QCascade, without ClpX, and 1 kb transposon integration.

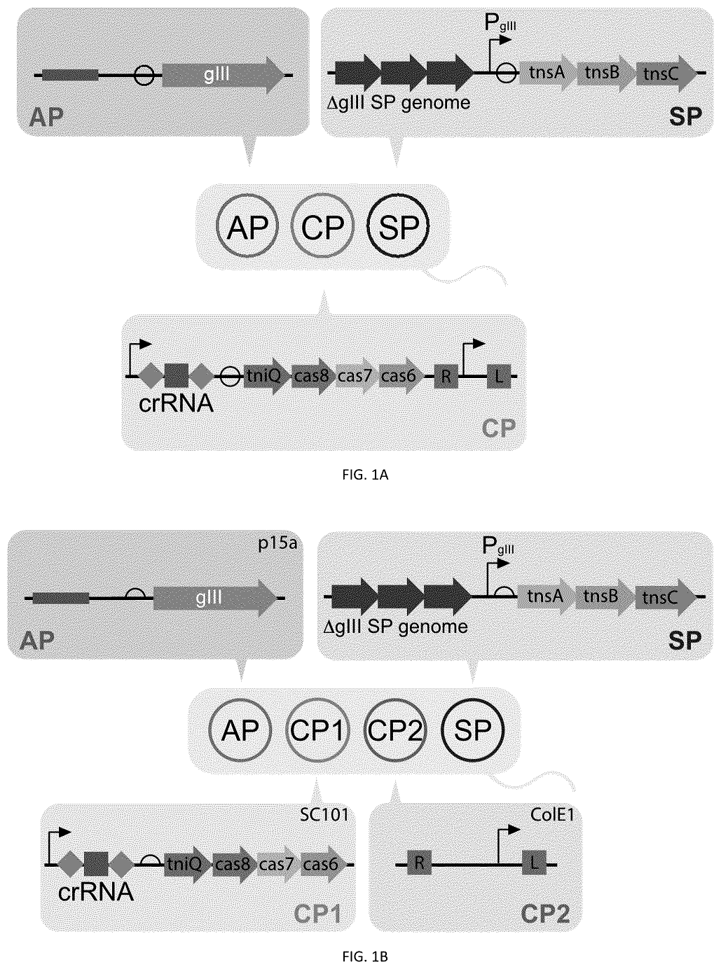

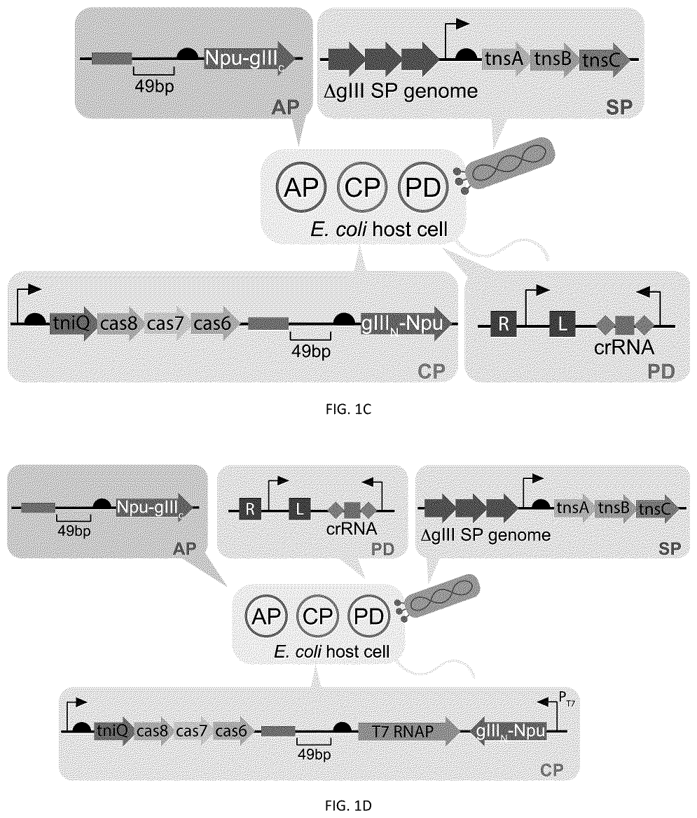

### DETAILED DESCRIPTION

In bacteria and archaea, CRISPR/Cas systems provide immunity by incorporating fragments of invading phage, virus, and plasmid DNA into CRISPR loci and using corresponding CRISPR RNAs (“crRNAs”) to guide the degradation of homologous sequences. Transcription of a CRISPR locus produces a “pre-crRNA,” which is processed to yield crRNAs containing spacer-repeat fragments that guide effector nuclease complexes to cleave dsDNA sequences complementary to the spacer. Several different types of CRISPR systems are known, (e.g., type I, type II, or type III), and classified largely based on the Cas protein type and the use of a proto-spacer-adjacent motif (PAM) for selection of proto-spacers in invading DNA.

Although RNA-guided targeting typically leads to endonucleolytic cleavage of the bound substrate, recent studies have uncovered a range of noncanonical pathways in which CRISPR protein-RNA effector complexes have been naturally repurposed for alternative functions. For example, some Type I (Cascade) and Type II (Cas9) systems leverage truncated guide RNAs to achieve potent transcriptional repression without cleavage, and other Type I (Cascade) and Type V (Cas12) systems lie inside unusual bacterial Tn7-like transposons and lack nuclease components altogether.

The present disclosure provides for transposon-associated and related Cas proteins for use in CRISPR-Tn systems, e.g., Type I (Cascade) and Type V (Cas12) systems. The present disclosure also provides for methods of creating the transposon-associated and related Cas proteins, as well as methods of using the transposon-associated and related Cas proteins or nucleic acid molecules encoding the transposon-associated and related Cas proteins in applications including editing a nucleic acid molecule, e.g., a genome. Methods of engineering the transposon-associated and related Cas proteins described herein may comprise phage-assisted continuous evolution (PACE) or phage-assisted non-continuous evolution (e.g., PANCE). The disclosure also provides methods for nucleic acid modification (e.g., RNA-guided DNA integration) utilizing engineered CRISPR-transposon systems comprising one or more of the disclosed transposon-associated and related Cas proteins.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of cell and tissue culture, molecular biology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The term “accessory plasmid,” as used herein, refers to a plasmid comprising a gene required for the generation of infectious viral particles under the control of a conditional promoter. In the context of continuous evolution described herein, transcription from the conditional promoter of the accessory plasmid is typically activated by a function of the protein(s) to be evolved. Accordingly, the accessory plasmid serves the function of conveying a competitive advantage to those viral vectors in a given population of viral vectors that carry a gene of interest able to activate the conditional promoter. Only viral vectors carrying an “activating” version of the protein(s) of interest will be able to induce expression of the gene required to generate infectious viral particles in the host cell, and, thus, allow for packaging and propagation of the viral genome in the flow of host cells. Vectors carrying non-activating versions of the protein of interest, on the other hand, will not induce expression of the gene required to generate infectious viral vectors, and, thus, will not be packaged into viral particles that can infect fresh host cells.

The term “contacting” as used herein refers to bring or put in contact, to be in or come into contact. The term “contact” as used herein refers to a state or condition of touching or of immediate or local proximity. Contacting a composition to a target destination, such as, but not limited to, an organ, tissue, cell, or tumor, may occur by any means of administration known to the skilled artisan.

The term “continuous evolution,” as used herein, refers to an evolution process in which a population of nucleic acids encoding a protein of interest is subjected to multiple rounds of (a) replication, (b) mutation, and (c) selection to produce a desired evolved protein that is different from the original protein of interest. The multiple rounds can be performed without investigator intervention, and the steps (a)-(c) can be carried out simultaneously. Typically, the evolution procedure is carried out in vitro, for example, using cells in culture as host cells. In general, a continuous evolution process provided herein relies on a system in which a gene encoding a protein of interest is provided in a viral vector that undergoes a life-cycle including replication in a host cell and transfer to another host cell, wherein a critical component of the life-cycle, e.g., a gene essential for the generation of infectious viral particles, is deactivated and reactivation of the component is dependent upon an activity of the protein of interest that is a result of a mutation in the viral vector.

The term “gene” refers to a DNA sequence that comprises control and coding sequences necessary for the production of an RNA having a non-coding function (e.g., a ribosomal or transfer RNA), a polypeptide, or a precursor of any of the foregoing. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this disclosure, it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. For example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

The terms “high copy number plasmid” and “low copy number plasmid” are art-recognized, and those of skill in the art will be able to ascertain whether a given plasmid is a high or low copy number plasmid. In some embodiments, a low copy number accessory plasmid is a plasmid exhibiting an average copy number of plasmid per host cell in a host cell population of about 5 to about 100. In some embodiments, a very low copy number accessory plasmid is a plasmid exhibiting an average copy number of plasmid per host cell in a host cell population of about 1 to about 10. In some embodiments, a very low copy number accessory plasmid is a single-copy per cell plasmid. In some embodiments, a high copy number accessory plasmid is a plasmid exhibiting an average copy number of plasmid per host cell in a host cell population of about 100 to about 5000.

The term “homology” and “homologous” refers to a degree of identity. There may be partial homology or complete homology. A partially homologous sequence is one that is less than 100% identical to another sequence.

The term “host cell,” as used herein, refers to a cell that can host, replicate, and transfer a phage vector useful for a continuous evolution process as provided herein. In embodiments where the vector is a viral vector, a suitable host cell is a cell that can be infected by the viral vector, can replicate it, and can package it into viral particles that can infect fresh host cells. A cell can host a viral vector if it supports expression of genes of viral vector, replication of the viral genome, and/or the generation of viral particles. One criterion to determine whether a cell is a suitable host cell for a given viral vector is to determine whether the cell can support the viral life cycle of a wild-type viral genome that the viral vector is derived from. For example, if the viral vector is a modified M13 phage genome, as provided in some embodiments described herein, then a suitable host cell would be any cell that can support the wild-type M13 phage life cycle. Suitable host cells for viral vectors useful in continuous evolution processes are well known to those of skill in the art, and the disclosure is not limited in this respect. In some embodiments, the viral vector is a phage and the host cell is a bacterial cell. In some embodiments, the host cell is an E. coli cell. Suitable E. coli host strains will be apparent to those of skill in the art, and include, but are not limited to, New England Biolabs (NEB) Turbo, ToplOF′, DH12S, ER2738, ER2267, and XL1-Blue MRF′. These strain names are art recognized and the genotype of these strains has been well characterized. It should be understood that the above strains are exemplary only and that the invention is not limited in this respect. The term “fresh,” as used herein interchangeably with the terms “non-infected” or “uninfected” in the context of host cells, refers to a host cell that has not been infected by a viral vector comprising a gene of interest as used in a continuous evolution process provided herein. A fresh host cell can, however, have been infected by a viral vector unrelated to the vector to be evolved or by a vector of the same or a similar type but not carrying the gene of interest. In some embodiments, the host cell is a prokaryotic cell, for example, a bacterial cell. In some embodiments, the host cell is an E. coli cell. In some embodiments, the host cell is a eukaryotic cell, for example, a yeast cell, an insect cell, or a mammalian cell. The type of host cell, will, of course, depend on the viral vector employed, and suitable host cell/viral vector combinations will be readily apparent to those of skill in the art.

As used herein, the term “hybridization” is used in reference to the pairing of complementary nucleic acids. Hybridization and the strength of hybridization (e.g., the strength of the association between the nucleic acids) is influenced by such factors as the degree of complementary between the nucleic acids, stringency of the conditions involved, and the Tₘ of the formed hybrid. Hybridization methods involve the annealing of one nucleic acid to another, complementary nucleic acid, e.g., a nucleic acid having a complementary nucleotide sequence. The ability of two polymers of nucleic acid containing complementary sequences to find each other and “anneal” or “hybridize” through base pairing interaction is a well-recognized phenomenon. The initial observations of the “hybridization” process by Marmur and Lane, Proc. Natl. Acad. Sci. USA, 46:453 (1960) and Doty et al., Proc. Natl. Acad. Sci. USA, 46:461 (1960), have been followed by the refinement of this process into an essential tool of modern biology. For example, hybridization and washing conditions are now well known and exemplified in Sambrook et al., supra. The conditions of temperature and ionic strength determine the “stringency” of the hybridization.

The term “lagoon,” as used herein, refers to a culture vessel or bioreactor through which a flow of host cells is directed. When used for a continuous evolution process as described herein, a lagoon typically holds a population of host cells and a population of viral vectors replicating within the host cell population, wherein the lagoon comprises an outflow through which host cells are removed from the lagoon and an inflow through which fresh host cells are introduced into the lagoon, thus replenishing the host cell population.

As used herein, “nucleic acid” or “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41 (14): 4503-4510 (2002)) and U.S. Pat. No. 5,034,506), locked nucleic acid (LNA; see Wahlestedt et al., Proc. Natl. Acad. Sci. U.S.A., 97:5633-5638 (2000)), cyclohexenyl nucleic acids (see Wang, J. Am. Chem. Soc., 122:8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

Nucleic acid or amino acid sequence “identity,” as described herein, can be determined by comparing a nucleic acid or amino acid sequence of interest to a reference nucleic acid or amino acid sequence. A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215 (3): 403-410 (1990), Beigert et al., Proc. Natl. Acad. Sci. USA, 106 (10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21 (7): 951-960 (2005), Altschul et al., Nucleic Acids Res., 25 (17): 3389-3402 (1997), and Gusfield, Algorithms on Strings, Trees and Sequences, Cambridge University Press, Cambridge UK (1997)).

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

The term “phage,” as used herein interchangeably with the term “bacteriophage,” refers to a virus that infects bacterial cells. Typically, phages consist of an outer protein capsid enclosing genetic material. The genetic material can be ssRNA, dsRNA, ssDNA, or dsDNA, in either linear or circular form. Phages and phage vectors are well known to those of skill in the art and non-limiting examples of phages that are useful for carrying out the methods provided herein are λ (Lysogen), T2, T4, T7, T12, R17, M13, MS2, G4, PI, P2, P4, Phi X174, N4, Φ6, and Φ29. In certain embodiments, the phage utilized in the present invention is M13. Additional suitable phages and host cells will be apparent to those of skill in the art and the invention is not limited in this aspect. For an exemplary description of additional suitable phages and host cells, see Elizabeth Kutter and Alexander Sulakvelidze: Bacteriophages: Biology and Applications. CRC Press; 1st edition (December 2004), ISBN: 0849313368; Martha R. J. Clokie and Andrew M. Kropinski: Bacteriophages: Methods and Protocols, Volume 1: Isolation, Characterization, and Interactions (Methods in Molecular Biology) Humana Press; 1st edition (December 2008), ISBN: 1588296822; Martha R. J. Clokie and Andrew M. Kropinski: Bacteriophages: Methods and Protocols, Volume 2: Molecular and Applied Aspects (Methods in Molecular Biology) Humana Press; 1st edition (December 2008), ISBN: 1603275649; all of which are incorporated herein in their entirety by reference for disclosure of suitable phages and host cells as well as methods and protocols for isolation, culture, and manipulation of such phages).

The terms “phage-assisted continuous evolution” or “PACE,” as used herein, refer to continuous evolution that employs phage as viral vectors. PACE technology has been described previously, for example, in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO2012/088381 on Jun. 28, 2012; International PCT Application, PCT/US2015/057012, filed Oct. 22, 2015, published as WO2016/077052 on May 19, 2016; International PCT Application, PCT/US2016/043513, filed Jul. 22, 2016, published as WO2017/015545 on Jan. 26, 2017; International PCT Application, PCT/US2016/058344, filed Oct. 22, 2016, published as WO2017/070632 on Apr. 27, 2017; and U.S. Pat. No. 9,267,127, granted based one U.S. application Ser. No. 13/922,812, filed Jun. 20, 2013, all of which are incorporated herein by reference.

The terms “phage-assisted non-continuous evolution” or “PANCE,” as used herein, refers to non-continuous evolution that employs phage as viral vectors. The general concept of PANCE technology has been described, for example, in Suzuki T. et al, Crystal structures reveal an elusive functional domain of pyrrolysyl-tRNA synthetase, Nat Chem Biol. 13 (12): 1261-1266 (2017), incorporated herein by reference in its entirety. Briefly, PANCE is a simplified technique for rapid in vivo directed evolution using serial flask transfers of evolving ‘selection phage’ (SP), which contain a gene of interest to be evolved, across fresh E. coli host cells, thereby allowing genes inside the host E. coli to be held constant while genes contained in the SP continuously evolve. Following phage growth, an aliquot of infected cells is used to transfect a subsequent flask containing host E. coli. This process is continued until the desired phenotype is evolved, for as many transfers as required. Serial flask transfers have long served as a widely-accessible approach for laboratory evolution of microbes, and, more recently, analogous approaches have been developed for bacteriophage evolution. In general, the PANCE system features lower stringency than the PACE system.

The terms “protein,” “peptide,” and “polypeptide” are used interchangeably herein, and refer to a polymer of amino acid residues linked together by peptide bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, engineered, or synthetic, or any combination thereof. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

As used herein, the terms “providing,” “administering,” and “introducing,” are used interchangeably herein and refer to the placement of the systems of the disclosure into a cell, organism, or subject by a method or route which results in at least partial localization of the system to a desired site. The systems can be administered by any appropriate route which results in delivery to a desired location in the cell, organism, or subject.

The term “selection phage,” as used herein interchangeably with the term “selection plasmid,” refers to a modified phage that comprises a gene of interest to be evolved and lacks a full-length gene encoding a protein required for the generation of infectious phage particles. For example, some M13 selection phages provided herein comprise a nucleic acid sequence encoding one or more transposases to be evolved, e.g., under the control of an M13 promoter, and lack all or part of a phage gene encoding a protein required for the generation of infectious phage particles, e.g., gI, gII, gIII, gIV, gV, gVI, gVII, gVIII, gIX, or gX, or any combination thereof. For example, some M13 selection phages provided herein comprise a nucleic acid sequence encoding one or more transposases to be evolved, e.g., under the control of an M13 promoter, and lack all or part of a gene encoding a protein required for the generation of infective phage particles, e.g., the gIII gene encoding the pIII protein.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice, guinea pigs, and the like. Examples of non-mammals include, but are not limited to, birds, fish, and the like. In one embodiment of the methods and compositions provided herein, the mammal is a human.

A “vector” or “expression vector” is a replicon, such as plasmid, phage, virus, or cosmid, to which another DNA segment, e.g., an “insert,” may be attached or incorporated so as to bring about the replication of the attached segment in a cell.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### CRISPR-Transposon Protein Components

Disclosed herein are modified transposon-associated proteins and Cas proteins. Further disclosed are nucleic acids and vectors comprising a sequence encoding the modified transposon-associated proteins and Cas proteins.

The modified transposon-associated proteins and/or Cas proteins may confer desirable traits (e.g., increased stability, increased activity) not found in the wild-type versions of the proteins. In some embodiments, the modified proteins show increased activity or utility in modifying a target nucleic acid compared to a protein not having the disclosed modifications. In some embodiments, the modified proteins increase target DNA binding activity compared to a protein not having the disclosed modifications. In some embodiments, the modified proteins increase nucleic acid integration activity at a target nucleic acid compared to a protein not having the disclosed modifications. In some embodiments, the modified proteins increase nucleic acid integration activity or efficiency at a target nucleic acid in vivo (e.g., in a prokaryotic or eukaryotic cell, in a subject) compared to a protein not having the disclosed modifications. In some embodiments, combinations of the modified transposon-associated proteins and/or Cas proteins confer desirable traits. In some embodiments, combinations of one or more of the modified transposon-associated proteins and/or Cas proteins with one or more wild-type transposon-associated proteins and/or Cas proteins confer desirable traits.

Provided herein are polypeptides comprising one or more amino acid sequences having at least 70% (e.g., having at least 75%, at least 80%, at least 85%, at least 90%, at least 92%, at least 95%, at least 96%, at least 97%, at least 98%, or at least 99%) (e.g., having at least 75%, at least 80%, at least 85%, at least 90%, at least 92%, at least 95%, at least 96%, at least 97%, at least 98%, or at least 99%) identity to any of SEQ ID NOs: 1-14. In some embodiments, the polypeptides have one or more amino acid substitutions, deletions, or additions relative to SEQ ID NOs: 1-14. In some embodiments, the polypeptides have one or more amino acid substitutions, deletions, or additions as shown in Tables 1-4 relative to SEQ ID NOs: 1-14.

Any of the proteins described or referenced herein may comprise one or more amino acid substitutions as compared to the recited sequences. An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or He), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gin), lysine (K or Lys), and arginine (R or Arg).

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra). Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free-OH can be maintained, and glutamine for asparagine such that a free-NH₂ can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

Provided herein is a polypeptide having at least 70% (e.g., having at least 75%, at least 80%, at least 85%, at least 90%, at least 92%, at least 95%, at least 96%, at least 97%, at least 98%, or at least 99%) identity to SEQ ID NO: 1 and one or more amino acid substitutions at positions: 2, 3, 5, 28, 57, 77, 80, 107, 110, 116, 122, 142, 155, 161, 166, 173, 177, 185, 211, 216, 227, and 230, relative to SEQ ID NO: 1. In some embodiments, the polypeptide comprises an amino acid sequence having one or more amino acid substitutions of: A2T, T3I, L5S, T28A, A57T, F77L, Y80D, K107M, K107R, Y110C, Y110D, D116G, E122A, D142E, M155I, K161R, N166D, K173E, Y177N, Y177D, C185R, D211Y, K216E, A227P, G230D and G230S, relative to SEQ ID NO: 1.

In some embodiments, the polypeptide comprises an amino acid sequence having amino acid substitutions at positions: 2 and 230; 107 and 166; 107, 166, and one or both of: 2 and 227; 211 and 110 or 142; 110, 155 and 230; 122 and 155; 155 and 177, relative to SEQ ID NO: 1. In some embodiments, the polypeptide comprises an amino acid sequence having amino acid substitutions of: A2T, and G230D or G230S, relative to SEQ ID NO: 1. In some embodiments, the polypeptide comprises an amino acid sequence having amino acid substitutions of: K107M and N166D, relative to SEQ ID NO: 1. In some embodiments, the polypeptide further comprises amino acid substitutions of: A2T and/or Y177N or Y177D, relative to SEQ ID NO: 1. In some embodiments, the polypeptide comprises an amino acid sequence having amino acid substitutions of: D211Y and Y110C, Y110D, or D142E, relative to SEQ ID NO: 1. In some embodiments, the polypeptide comprises an amino acid sequence having amino acid substitutions of: Y110C or Y110D, M155I, and G230D or G230S, relative to SEQ ID NO: 1. In some embodiments, the polypeptide comprises an amino acid sequence having amino acid substitutions of: E122A and M155I, relative to SEQ ID NO: 1. In some embodiments, the polypeptide comprises an amino acid sequence having amino acid substitutions of: M155I and Y177N or Y177D, relative to SEQ ID NO: 1.

Provided herein is a polypeptide having at least 70% (e.g., having at least 75%, at least 80%, at least 85%, at least 90%, at least 92%, at least 95%, at least 96%, at least 97%, at least 98%, or at least 99%) identity to SEQ ID NO: 2 and one or more amino acid substitutions at positions: 2, 5, 22, 24, 25, 29, 75, 141, 199, 215, 319, 347, 364, 370, 383, 439, 454, 458, 485, 509, 533, 538, 565, 581, 586, 595, 596, 597, and 600, relative to SEQ ID NO: 2. In some embodiments, the polypeptide comprises an amino acid sequence having one or more amino acid substitutions of: A2T, A2S, G5R, S22P, E24D, L25I, A29S, P75T, I141T, V199I, S215R, D319V, Y347F, S364N, E370K, N383D, V439A, E454D, E454G, S458N, V485F, R509G, D533A, A538V, H565Y, A581T, H586L, N595K, D596N, D597N, D597Y, and I600V, relative to SEQ ID NO: 2. In some embodiments, the polypeptide comprises an amino acid sequence having amino acid substitutions at positions: 2 and 597; 24 and 25; 24, 25, 458, 509, 565, and 600; 75 and 597; 141, 454, 533 and 595; 581, 370, and 454; 370 and 581; 370 and 454; 458 and 509; 458, 509 and 565; 458, 509, 565, and 600; 565, 586, and 596; or 565, 509, 458, 600 and at least one of 24, 25, 29, 215, 319, 364, 383, and 586, relative to SEQ ID NO: 2.

In some embodiments, the polypeptide comprises an amino acid sequence having amino acid substitutions of: A2T or A2S, and D597N or D597Y, relative to SEQ ID NO: 2. In some embodiments, the polypeptide comprises an amino acid sequence having amino acid substitutions of: E24D and L25I, relative to SEQ ID NO: 2. In some embodiments, the polypeptide comprises an amino acid sequence having amino acid substitutions of: E24D and L25I, S458N, R509G, H565Y, and I600V, relative to SEQ ID NO: 2. In some embodiments, the polypeptide comprises an amino acid sequence having amino acid substitutions of: P75T and D597N or D597Y, relative to SEQ ID NO: 2. In some embodiments, the polypeptide comprises an amino acid sequence having amino acid substitutions of: E454D or E454G, D533A, N595K, relative to SEQ ID NO: 2. In some embodiments, the polypeptide comprises an amino acid sequence having amino acid substitutions of: E370K, E454D or E454G, and A581T, relative to SEQ ID NO: 2. In some embodiments, the polypeptide comprises an amino acid sequence having amino acid substitutions of: E370K, and A581T, E454D, or E454G, relative to SEQ ID NO: 2. In some embodiments, the polypeptide comprises an amino acid sequence having amino acid substitutions of: S458N and R509G, relative to SEQ ID NO: 2. In some embodiments, the polypeptide further comprises amino acid substitutions of H565Y and/or I600V. In some embodiments, the polypeptide comprises an amino acid sequence having amino acid substitutions of: H565Y, H586L, and D596N, relative to SEQ ID NO: 2. In some embodiments, the polypeptide comprises an amino acid sequence having amino acid substitutions of: H565Y, R509G, S458N, I600V and at least one of E24D, L25I, A29S, S215R, D319V, S364N, N383D, and H586L, relative to SEQ ID NO: 2.

Provided herein is a polypeptide having at least 70% (e.g., having at least 75%, at least 80%, at least 85%, at least 90%, at least 92%, at least 95%, at least 96%, at least 97%, at least 98%, or at least 99%) identity to SEQ ID NO: 3 and one or more amino acid substitutions at positions: 9, 15, 16, 18, 21, 64, 81, 86, 87, 99, 109, 142, 147, 153, 168, 180, 216, 230, 285, and 304, relative to SEQ ID NO: 3. In some embodiments, the polypeptide comprises an amino acid sequence having one or more amino acid substitutions of: 19V, A15V, F16Y, S18F, S21N, N64D, H81Y, D86Y, N87K, V991, E109D, E142K, V147I, N153D, I168M, A180E, A216S, L230F, K285E, and R304R, relative to SEQ ID NO: 3. In some embodiments, the polypeptide comprises an amino acid sequence having amino acid substitutions at positions: 142 and 216, relative to SEQ ID NO: 3. In some embodiments, the polypeptide comprises an amino acid sequence having amino acid substitutions of: E142K and A216S, relative to SEQ ID NO: 3.

Provided herein is a polypeptide having at least 70% (e.g., having at least 75%, at least 80%, at least 85%, at least 90%, at least 92%, at least 95%, at least 96%, at least 97%, at least 98%, or at least 99%) identity to SEQ ID NO: 4 and one or more amino acid substitutions at positions: 4, 5, 9, 10, 12, 21, 23, 25, 26, 31, 32, 34, 35, 37, 41, 45, 47, 48, 51, 52, 55, 60, 61, 65, 67, 69, 72, 75, 79, 80, 82, 87, 88, 90, 91, 93, 94, 96, 98, 99, 100, 103, 106, 108, 113, 116, 125, 126, 128, 129, 135, 139, 143, 146, 147, 149, 153, 154, 156, 158, 159, 160, 162, 164, 166, 167, 168, 169, 170, 177, 179, 180, 182, 183, 185, 187, 188, 190, 191, 192, 193, 195, 196, 200, 204, 207, and 208, relative to SEQ ID NO: 4. In some embodiments, the polypeptide comprises an amino acid sequence having one or more amino acid substitutions of: R4K, N5K, P9S, A10P, N12D, T21I, V23M, S25N, S25R, V26M, V26G, S31N, S32I, E34A, F35L, A37D, H41L, D45N, I47V, E48G, G51V, S521, E55K, E55D, E60K, F61L, S65T, S65A, P67T, P67L, P67S, P67H, T69A, A72V, A72D, S75I, S75R, S75T, K79E, T80P, K82E, K87R, P88L, P88T, P88A, S90F, K91N, K91E, A93T, A93S, S94N, L96P, R98Q, A99D, A99V, E100K, A103T, A106T, S108A, I113F, V116F, V116I, V125M, V125A, N126T, 1128V, 1128L, L129P, L135M, S139N, S139G, G143V, G143C, G146D, G146S, I147V, K149E, K149T, K149R, S153I, S153R, S153N, F154C, H156R, H156L, S158N, S158R, G159V, V160A, K162R, N164D, I166L, S167I, S168I, S168R, S168N, Q169R, V170M, V170G, V170L, T177I, T177A, S179R, F180C, F180L, F182C, F182L, G183S, M185I, K187R, G188D, V190I, K191N, A192S, D193N, G195V, G195D, G195S, C196W, T200A, T204I, A207V, A207T, and T208I, relative to SEQ ID NO: 4.

In some embodiments, the polypeptide comprises an amino acid sequence having amino acid substitutions at positions: 108 and 47 or 208; 170 and 207; 88 and 147; 47, 88 and 147; 88, 147, 170, and 182; 88, 147, 170, 182, and 51 or 180; 88, 147, and 154; 88, 128, 147, 170, and 182; or 170, 207, and 108, relative to SEQ ID NO: 4. In some embodiments, the polypeptide comprises an amino acid sequence having amino acid substitutions of: S108A, and I47V or T208I, relative to SEQ ID NO: 4. In some embodiments, the polypeptide comprises an amino acid sequence having amino acid substitutions of: V170M, and A207V or A207T, relative to SEQ ID NO: 4. In some embodiments, the polypeptide further comprises amino acid substitutions of: S108A, V170M, and A207V or A207T, relative to SEQ ID NO: 4.

Provided herein is a polypeptide having at least 70% (e.g., having at least 75%, at least 80%, at least 85%, at least 90%, at least 92%, at least 95%, at least 96%, at least 97%, at least 98%, or at least 99%) identity to SEQ ID NO: 5 and one or more amino acid substitutions at positions: 1, 2, 4, 5, 8, 9, 10, 11, 12, 13, 14, 15, 16, 17, 19, 20, 21, 22, 23, 24, 25, 26, 27, 28, 29, 30, 32, 33, 36, 37, 39, 40, 41, 42, 43, 44, 45, 49, 52, 55, 56, 58, 60, 62, 63, 67, 71, 74, 76, 78, 79, 80, 81, 82, 83, 84, 85, 86, 87, 88, 89, 91, 92, 95, 97, 100, 101, 104, 106, 110, 112, 113, 115, 117, 119, 120, 124, 125, 127, 129, 130, 131, 134, 139, 142, 144, 145, 146, 147, 149, 150, 155, 156, 157, 158, 159, 163, 164, 165, 167, 169, 173, 174, 176, 181, 182, 186, 187, 190, 195, 197, 198, 205, 208, 209, 211, 215, 218, 223, 226, 227, 231, 232, 235, 239, 246, 248, 250, 259, 260, 261, 262, 263, 267, 269, 273, 274, 277, 278, 280, 281, 282, 283, 285, 287, 288, 290, 295, 298, 302, 303, 307, 313, 316, 317, 320, 323, 325, 331, 332, 339, 345, 348, 349, 352, 353, 354, 356, 361, 362, 363, 364, 365, 366, 367, 369, 370, 371, 372, 373, 375, 376, 380, 383, 385, 386, 389, 390, 392, 396, 397, 399, 402, 403, 404, 407, 408, 410, 411, 412, 413, 414, 415, 416, 421, 422, 423, 424, 425, 426, 427, 428, 429, 430, 431, 434, 435, 437, 440, 443, 445, 446, 448, 450, 452, 456, 459, 460, 463, 464, 470, 472, 473, 494, 495, 498, 501, 502, 504, 505, 506, 508, 509, 510, 512, 513, 514, 517, 520, 521, 522, 525, 526, 527, 530, 531, 532, 533, 535, 537, 538, 540, 541, 542, 543, 544, 545, 546, 547, 548, 549, 550, 551, 552, 553, 554, 556, 557, 558, 559, 560, 561, 562, 563, 564, 565, 567, 568, 569, 570, 571, 574, 575, 576, 580, 582, 583, 584, 585, 586, 587, 588, 589, 590, 591, 592, 593, 594, 595, 596, 597, 599, 600, 601, 602, 603, 604, 606, 607, 608, 611, 613, 618, 620, and 656, relative to SEQ ID NO: 5.

In some embodiments, the polypeptide comprises an amino acid sequence having one or more amino acid substitutions of: M1V, M1I, M1L, T2I, T2A, F4L, F5L, F8L, F8V, F8S, D9N, E10K, E10D, S11I, S11R, S11G, L12P, V13M, V13G, V13E, V13L, P14L, L15Q, K16N, K16R, P17T, P17L, P17S, T191, T19S, T19A, T19P, P20S, P20L, T21A, Q22R, Y23H, V24M, K25R, L26M, D27A, D27G, D28N, D28Y, A29T, A29V, N30K, I32F, I32S, Q33H, L36M, D37A, D37Y, F39L, S40P, D41E, T42I, T42K, T42A, F43L, F43S, F43V, K44N, N45D, N45S, Q49R, K52Q, S55A, T56A, D58E, K60Q, S62T, R63K, R63G, Q67R, Q67H, Q67K, D71Y, K74R, E76K, F78C, K79R, G80V, G80D, G81S, G81V, G81D, D82N, V83G, V83M, V83A, V84A, V84G, R85G, R85K, P86L, N87S, R89C, V91G, V91A, A92V, A92T, R95K, K97R, E100D, S101A, D104V, A106D, A106T, D110N, N112H, H113Y, M115R, N117Y, T119A, N120D, N120K, N120S, G124V, D125N, D125E, K127R, F129L, D130N, K131M, E134D, E134G, A139S, A139T, P142S, 1144V, A145S, A145T, T146A, A147V, Q149R, Y150H, I155L, V156A, V156L, V156M, K157V, E158A, N159S, V163G, E164A, E164G, E164D, G165D, I167V, I169L, I169T, N173S, N173H, N173T, A174S, A174T, N176D, A181S, I182L, I182V, I182T, A186E, A186T, V187G, V187A, A190T, A190S, F195S, A197P, D198G, D198N, A205S, V208M, P209T, T211I, E215D, E218D, P223S, P223H, L226V, I227V, D231N, E232K, I235V, I235T, R239G, I246V, V248E, V248M, S250I, S259N, Y260C, K261R, S262N, P263L, S267N, A269V, T273I, T273N, H274Y, K277N, K277R, P278S, S280T, L281M, D282E, D282N, A283T, A283S, N285S, E287D, L288M, N290K, F295S, F298I, F298S, V302I, V303M, A307S, N313S, H316R, A317V, S320N, S320R, I323L, I325V, R331K, K332E, I339V, V345L, V345M, E348K, Y349H, Y349D, Y349N, Y349C, P352S, P352T, E353Q, E353D, L354M, G356S, N361D, I362V, I362T, L363P, L363T, L363M, E364G, K365R, E366G, E367G, K369N, K369E, K369M, P370S, E371K, V372M, D373G, I375V, M376I, T380P, T380A, E383K, E383D, F385L, H386Y, I389V, A390V, A390I, V392I, D396N, D396G, D396K, S397P, S399N, S399G, T402I, R403G, R403I, R403K, R403S, I404T, I404V, K407R, K407E, R408K, Q410K, Q410H, Q410R, Q411H, G412V, F413L, D414N, A415V, A415T, Y416C, M421I, N422K, E423K, E423D, E424A, E425K, E426D, T427A, T427S, R428K, F429L, S430A, M431L, R434H, R434C, R434S, I435V, D437G, D437N, T440S, T440I, R443C, G445S, F446L, F446I, Y448C, E450D, E450G, M452I, T456P, T456A, T456I, A459T, D460N, K463N, H464N, H464R, H464S, E470K, V472M, V472A, K473D, K473N, E494D, E494G, S495A, E498A, E498K, C501Y, T502I, T502S, P504S, P504L, T505A, G506Y, G506D, G506L, G506S, T508A, D509E, D509Y, C510Y, S512N, I513L, I513V, I513F, Y514H, K517M, K517N, K517Q, K520R, K521N, I522T, I522V, I522F, E525K, V526E, V526M, I527V, S530N, S530R, K531T, D532G, D532Y, S533Y, G535D, A537T, K538R, K538N, R540K, R540G, M541L, A542T, I543L, H544R, E545A, R546G, R546K, V547M, K548Q, K548R, Q549K, Q549R, E550A, Q551K, E552D, E552K, V553I, F554V, E556K, E556G, S557A, K558R, T559P, T559I, T559A, K560R, A561T, A561G, K562R, K562N, I563L, T564I, A565S, A565V, K567R, K568N, K568R, Q569K, Q569L, Q569R, A570V, Q571R, D574N, V575M, V575A, S576R, T580I, T580A, T582I, T582S, I583V, K584R, V585M, S586P, S586A, S586F, E587A, E588K, E588G, E588D, S5891, S589R, S589N, A590S, A590T, A591V, P592L, V593M, V593A, Q594L, K595R, K595N, H596Y, H596L, H596P, I597T, I597V, N599H, D600L, D600N, D600G, D600V, N601S, N601K, S602A, S602P, S602Y, D603A, D603V, D604G, D604Y, D604N, D606A, D606V, D606Y, D607Y, D607E, D608N, A611T, E613D, R618I, T620P, and A656V, relative to SEQ ID NO: 5.

In some embodiments, the polypeptide comprises an amino acid sequence having amino acid substitutions at positions: 4, 23 and 590; 19, 169, and 549; 43 and 415; 80 and 593; 80, 144, 593, and 606; 1, 42, 80, 593, and 606; 42, 80, 593, and 606; 156 and 604; 283, 349, and 365; 283, 349, 365, 396, and 594; 283, 349, 365, 396, 594, 596, and 131; 352 and 390; 390, 396, and 594; 396 and 594; 456 and 502; 464 and 502; 464 and 17; 17, 235, 464, and 596; 235, 352, 396, 456, and 606; 415, 456, and 502; 456, 502, and 549; 169, 456, 502, and 549; 80, 456, 502, 593, and 606; 1, 42, 80, 456, 502, 593, and 606; 80, 144, 456, 502, 593, and 606; 19, 169, 456, 502 and 549; 43, 415, 456, and 502; 352, 390, 396, and 594; 352, 390, and 396; 283, 349, 396, and 594; 11, 55, 120, 362, 584, 600, and 604; 43, 84, 144, 349, and 517; 164 and 165; 164 and 173; 362 and 446; 352, 390, 396, 549, and 594; 352, 390, 396, 464, 549, and 594; 43, 349, 352, 390, 396, 464, 549, and 594; 43, 349, 352, 390, 396, 464, 549, 594 and one or more positions selected from 63, 145, 174, 182, 208, 410, 427, 456, 504, and 526; 43, 352, 390, 396, 464, 549, and 594; 43, 349, 352, 390, 396, 464, 549, 594, 410, 526, 415, and 502; 43, 349, 352, 390, 396, 464, 549, 594, 410, 526, 415, 502, and 21; 43, 349, 352, 390, 396, 464, 549, 594, 410, 526, 415, 502, and 67; 43, 349, 352, 390, 396, 464, 549, 594, 410, 526, 415, 502, 21, and 67; 43, 349, 352, 390, 396, 464, 549, 594, 410, 526, 174, 208, 427, 456, and 504; 43, 349, 352, 390, 396, 464, 549, 594, 410, 526, 415, 502, and 139; 43, 349, 352, 390, 396, 464, 549, 594, 410, 526, 415, 502, 339, and 446; 43, 349, 352, 390, 396, 464, 549, 594, 410, 526, 19, 460, 569, and 596; 43, 349, 352, 390, 396, 464, 549, 594, 410, 526, 460, 586, 588, and 608; 43, 349, 352, 390, 396, 464, 549, 594, 410, 526, and 460; 352, 390, 396, 549, 586, and 594; 63, 158, 352, 390, 396, 549, 586, and 594; 164, 165, 352, 363, 390, 396, 410, 549, 586, and 594; 164, 173, 352, 390, 396, 549, 586, and 594; 83, 352, 390, 396, 549, 586, and 594; 8, 43, 174, 349, 352, 390, 396, 427, 464, 549, and 594; or 283, 349, 365, 396, 594, 596, and 131, relative to SEQ ID NO: 5.

In select embodiments, the polypeptide comprises amino acid substitutions at positions: 352, 390, 396, 594, or any combination thereof, relative to SEQ ID NO: 5.

In select embodiments, the polypeptide comprises amino acid substitutions at positions: F43, Y349, P352, A390, D396, H464, Q549, Q594, and T456; F43, Y349, P352, A390, D396, H464, Q549, Q594, T456, and V526; F43, Y349, P352, A390, D396, H464, Q549, Q594, and P504; F43, Y349, P352, A390, D396, H464, Q549, Q594, and V526; F43, Y349, P352, A390, D396, H464, Q549, Q594, Q410, and V526; F43, Y349, P352, A390, D396, H464, Q549, Q594, A174, and T427; F43, Y349, P352, A390, D396, H464, Q549, Q594, and V208; F43, Y349, P352, A390, D396, H464, Q549, Q594, R63, A145, 1182, and V526; F43, Y349, P352, A390, D396, H464, Q549, Q594, Q410, V526, A415, and T502; F43, Y349, P352, A390, D396, H464, Q549, Q594, Q410, V526, A415, T502, and T21; F43, Y349, P352, A390, D396, H464, Q549, Q594, Q410, V526, A415, T502, and Q67; F43, Y349, P352, A390, D396, H464, Q549, Q594, Q410, V526, A415, T502, T21, and Q67; F43, Y349, P352, A390, D396, H464, Q549, Q594, Q410, V526, A174, V208, T427, T456, and P504; F43, Y349, P352, A390, D396, H464, Q549, Q594, Q410, V526, A174, V208, T427, T456, and P504; F43, Y349, P352, A390, D396, H464, Q549, Q594, Q410, V526, A415, T502, and A139; F43, Y349, P352, A390, D396, H464, Q549, Q594, Q410, V526, A415, T502, 1339, and F446; F43, Y349, P352, A390, D396, H464, Q549, Q594, Q410, V526, T19, D460, Q569, and H596; F43, Y349, P352, A390, D396, H464, Q549, Q594, Q410, V526, D460, S586, E588, and D608; F43, Y349, P352, A390, D396, H464, Q549, Q594, Q410, V526, and D460, relative to SEQ ID NO: 5.

In some embodiments, the polypeptide comprises an amino acid sequence having amino acid substitutions of: F4L, Y23H and A590S, relative to SEQ ID NO: 5. In some embodiments, the polypeptide comprises an amino acid sequence having amino acid substitutions of: T19P, I169L, and 549, relative to SEQ ID NO: 5. In some embodiments, the polypeptide comprises an amino acid sequence having amino acid substitutions of: F43L and A415V, relative to SEQ ID NO: 5. In some embodiments, the polypeptide comprises an amino acid sequence having amino acid substitutions of: G80D and V593M or V593A, relative to SEQ ID NO: 5. In some embodiments, the polypeptide comprises an amino acid sequence having amino acid substitutions of: G80D, 1144V, V593M or V593A, and D606A or D606V, relative to SEQ ID NO: 5. In some embodiments, the polypeptide comprises an amino acid sequence having amino acid substitutions of: M1V, M1I or M1L, T421 or T42A, G80D, V593M or V593A, and D606A or D606V, relative to SEQ ID NO: 5. In some embodiments, the polypeptide comprises an amino acid sequence having amino acid substitutions of: T42I or T42A, G80D, V593M or V593A, and D606A or D606V, relative to SEQ ID NO: 5. In some embodiments, the polypeptide comprises an amino acid sequence having amino acid substitutions of: V156A or V156M, and D604G or D604N, relative to SEQ ID NO: 5. In some embodiments, the polypeptide comprises an amino acid sequence having amino acid substitutions of: A283S or A283T, Y349H, and K365R, relative to SEQ ID NO: 5. In some embodiments, the polypeptide further comprises amino acid substitutions of: D396K and Q594K or Q594L, relative to SEQ ID NO: 5. In some embodiments, the polypeptide further comprises amino acid substitutions of: P352S or P352T, H596Y or H596L, and K131M, relative to SEQ ID NO: 5. In some embodiments, the polypeptide comprises an amino acid sequence having amino acid substitutions of: P352S or P352T and A390V, relative to SEQ ID NO: 5. In some embodiments, the polypeptide comprises an amino acid sequence having amino acid substitutions of: A390V, D396K, and Q594K or Q594L, relative to SEQ ID NO: 5. In some embodiments, the polypeptide comprises an amino acid sequence having amino acid substitutions of: D396K and Q594K or Q594L, relative to SEQ ID NO: 5. In some embodiments, the polypeptide comprises an amino acid sequence having amino acid substitutions of: T456P, T456I, or T456A and T502I, relative to SEQ ID NO: 5. In some embodiments, the polypeptide comprises an amino acid sequence having amino acid substitutions of: H464N or H464R and T502I, relative to SEQ ID NO: 5. In some embodiments, the polypeptide comprises an amino acid sequence having amino acid substitutions of: H464N or H464R and P17T, P17L, or P17S, relative to SEQ ID NO: 5. In some embodiments, polypeptide comprises an amino acid sequence having amino acid substitutions of: P17T, P17L, or P17S, I235V or I235T, H464N or H464R, and Q569K, Q569L or Q569R, relative to SEQ ID NO: 5. In some embodiments, the polypeptide comprises an amino acid sequence having amino acid substitutions of: I235V or I235T, P352S or P352T, D396K, T456P, T456I, or T456A, and D606A or D606V, relative to SEQ ID NO: 5. In some embodiments, the polypeptide comprises an amino acid sequence having amino acid substitutions of: A415V, T456P, and T502I, relative to SEQ ID NO: 5. In some embodiments, the polypeptide comprises an amino acid sequence having amino acid substitutions of: T456P, T502I, and Q549K, relative to SEQ ID NO: 5. In some embodiments, the polypeptide comprises an amino acid sequence having amino acid substitutions of: I169L, T456P, T502I, and Q549K, relative to SEQ ID NO: 5. In some embodiments, the polypeptide comprises an amino acid sequence having amino acid substitutions of: G80D, T456P, T502I, V593M, and D606A, relative to SEQ ID NO: 5. In some embodiments, the polypeptide comprises an amino acid sequence having amino acid substitutions of: M1V, T42I, G80D, T456P, T502I, V593M, and D606A, relative to SEQ ID NO: 5. In some embodiments, the polypeptide comprises an amino acid sequence having amino acid substitutions of: G80D, 1144V, T456P, T502I, V593M, and D606A, relative to SEQ ID NO: 5. In some embodiments, the polypeptide comprises an amino acid sequence having amino acid substitutions of: T19P, I169L, T456P, T502I, and Q549K, relative to SEQ ID NO: 5. In some embodiments, the polypeptide comprises an amino acid sequence having amino acid substitutions of: F43L, A415V, T456P, and T502I, relative to SEQ ID NO: 5. In some embodiments, the polypeptide comprises an amino acid sequence having amino acid substitutions of: P352T, A390V, D396N, and Q594L, relative to SEQ ID NO: 5. In some embodiments, the polypeptide comprises an amino acid sequence having amino acid substitutions of: P352T, A390V, and D396N, relative to SEQ ID NO: 5. In some embodiments, the polypeptide comprises an amino acid sequence having amino acid substitutions of: A283T, Y349H, D396N, and Q594L, relative to SEQ ID NO: 5. In some embodiments, the polypeptide comprises an amino acid sequence having amino acid substitutions of: A283T, Y349H, P352S, K365R, D396N, Q594L, H596L, and K131M, relative to SEQ ID NO: 5. In select embodiments, the polypeptide comprises an amino acid sequence having one or more amino acid substitutions of: P352T, A390V, D396N, and Q594L, relative to SEQ ID NO: 5.

In some embodiments, the polypeptide comprises an amino acid sequence having amino acid substitutions of: F43S, Y349N, P352T, A390V, D396N, H464R, Q549R, Q594L, and T456I, relative to SEQ ID NO: 5. In some embodiments, the polypeptide comprises an amino acid sequence having amino acid substitutions of: F43S, Y349N, P352T, A390V, D396N, H464R, Q549R, Q594L, T456P, and V526E, relative to SEQ ID NO: 5. In some embodiments, the polypeptide comprises an amino acid sequence having amino acid substitutions of: F43S, Y349D, P352T, A390V, D396N, H464R, Q549R, Q594L, and P504S, relative to SEQ ID NO: 5. In some embodiments, the polypeptide comprises an amino acid sequence having amino acid substitutions of: F43S, Y349N, P352T, A390V, D396N, H464R, Q549R, Q594L, and V526E, relative to SEQ ID NO: 5. In some embodiments, the polypeptide comprises an amino acid sequence having amino acid substitutions of: F43S, Y349N, P352T, A390V, D396N, H464R, Q549R, Q594L, Q410K, and V526E, relative to SEQ ID NO: 5. In some embodiments, the polypeptide comprises an amino acid sequence having amino acid substitutions of: F43S, Y349N, P352T, A390V, D396N, H464R, Q549R, Q594L, A174S, and T427S, relative to SEQ ID NO: 5. In some embodiments, the polypeptide comprises an amino acid sequence having amino acid substitutions of: F43S, Y349N, P352T, A390V, D396N, H464R, Q549R, Q594L, and V208M, relative to SEQ ID NO: 5. In some embodiments, the polypeptide comprises an amino acid sequence having amino acid substitutions of: F43S, Y349N, P352T, A390V, D396N, H464R, Q549R, Q594L, R63G, A145S, I182T, and V526E, relative to SEQ ID NO: 5.

In some embodiments, the polypeptide comprises an amino acid sequence having amino acid substitutions of: F43S, Y349N or Y349D, P352T, A390V, D396N, H464R, Q549R, Q594L, A415V and T502I, relative to SEQ ID NO: 5. In some embodiments, the polypeptide comprises an amino acid sequence having amino acid substitutions of: F43S, Y349N or Y349D, P352T, A390V, D396N, H464R, Q549R, Q594L, A415V, T502I, and T21A, relative to SEQ ID NO: 5. In some embodiments, the polypeptide comprises an amino acid sequence having amino acid substitutions of: F43S, Y349N or Y349D, P352T, A390V, D396N, H464R, Q549R, Q594L, A415V, T502I, and Q67K, relative to SEQ ID NO: 5. In some embodiments, the polypeptide comprises an amino acid sequence having amino acid substitutions of: F43S, Y349N or Y349D, P352T, A390V, D396N, H464R, Q549R, Q594L, A415V, T502I, T21A, and Q67K, relative to SEQ ID NO: 5.

Provided herein is a polypeptide having at least 70% (e.g., having at least 75%, at least 80%, at least 85%, at least 90%, at least 92%, at least 95%, at least 96%, at least 97%, at least 98%, or at least 99%) identity to SEQ ID NO: 6 and one or more amino acid substitutions at positions: 1, 2, 3, 5, 6, 7, 9, 11, 12, 14, 21, 22, 26, 27, 31, 35, 38, 43, 44, 46, 47, 54, 59, 60, 61, 64, 65, 67, 68, 71, 72, 74, 76, 79, 80, 81, 84, 89, 95, 102, 105, 109, 110, 111, 112, 113, 114, 116, 118, 119, 120, 123, 129, 130, 131, 132, 134, 142, 145, 146, 147, 148, 150, 154, 155, 166, 169, 178, 180, 181, 183, 184, 187, 190, 194, 197, 201, 204, 207, 209, 213, 219, 221, 225, 226, 227, 229, 232, 233, 234, 236, 238, 241, 246, 251, 252, 256, 257, 261, 263, 265, 267, 269, 271, 272, 274, 280, 281, 285, 286, 288, 291, 292, 296, 299, 301, 303, 304, 306, 307, 308, 310, 313, 314, 316, 317, 318, 319, 320, 323, 324, 326, 328, 330, 331, 332, 340, 341, 343, 344, 355, 412, 418, 427, 514, 1198, 1201, 1206, 1212, 1260, and 1282, relative to SEQ ID NO: 6. In some embodiments, the polypeptide comprises an amino acid sequence having one or more amino acid substitutions of: M1L, M1V, N2S, A3T, T5P, T5A, T5S, E6D, 17S, 17V, 19F, Q11R, L12M, N14D, N14S, M21I, H22P, H22Y, K26N, K26R, T27I, M31I, L35R, N38S, S43P, D44N, D44G, Q46L, C47S, T54I, S59T, H60Y, T61A, H64Y, Y65H, K67N, K67R, R68Q, A71G, T72A, N74D, S76C, S76Y, T791, M80I, P81S, V84L, R89L, A95D, A95T, A102T, E105D, E105K, S109N, S109R, S110P, Q111R, 1112T, K113N, K113E, K114N, K114M, K114E, G116D, K118N, K118R, T119I, D120V, K123N, L129M, I130V, K131R, A132S, K134M, K134N, F142V, L145M, I146T, E147K, F148S, S150F, R154K, Q155H, E166D, K169E, P178S, A180V, A181T, A181S, I183V, A184S, A184T, A184V, P187S, A190T, A190V, V194M, V194A, R197I, Y201N, L204M, D207N, K209N, Q213H, Q213V, A219S, K221N, D225N, V226E, P227T, K229E, S232N, K233N, K233R, N234H, T236A, A238V, A238S, A241S, E246D, K251N, H252Y, H252R, E256D, A257S, A261V, S263I, S263N, N265D, Y267C, E269K, E269D, K271E, K271R, H272Y, I274V, F280L, D281N, D281G, K285G, K286N, K288R, S291F, S291P, K292N, K296R, K296N, I299S, D301G, E303D, I304T, I304V, E306G, V307L, V307G, V307A, V307D, V307G, I308N, N310S, Y313H, N314K, N316K, N316D, A317D, L318Q, D319N, P320S, P320L, M323I, L324M, D326N, V328M, V328A, A330D, I331V, V332G, S340L, T341A, A343G, S344N, I355V, F412V, V418F, Y427C, R514K, S1198L, A1201V, G1206S, C1212G, F1260L, and V1282M, relative to SEQ ID NO: 6.

In some embodiments, the polypeptide does not include substitutions at one or more positions selected from: 6, 9, 28, 44, 64, 76, 80, 95, 110, 113, 114, 116, 118, 130, 132, 142, 155, 187, 190, 194, 221, 233, 234, 238, 261, 272, 280, 281, 299, 303, 304, 307, 308, 313, 316, and 328, relative to SEQ ID NO: 6. In some embodiments, the polypeptide does not include one or more substitutions selected from: E6D, 19F, I28T, D44N, D44G, H64Y, S76Y, M80I, A95T, S110P, K113N, K113E, K114N, K114E, G116D, K118N, K118R, I130V, A132S, F142V, Q155H, P187S, A190T, V194A, K221N, K233R, N234H, A238V, A261V, H272Y, F280L, D281G, I299D, E303F, I304T, V307S, I308N, Y313H, N316D, and V328M, relative to SEQ ID NO: 6.

In some embodiments, the polypeptide comprises an amino acid sequence having amino acid substitutions at positions: 2, 67, 95, and 226; 6 and 316; 38, 95, 303; 44 and 118; 67, 95, and 226; 44 and 76; 44, 76, and 118; 130, 234, 303; 118 and 1201; 118, 1201, and 44; 118, 1201, and 76; 130, 234, and 303; 154 and 269; 221 and 44; 44, 76, 130, 234, and 303; 44, 76, 118, and 1201; 197 and 314; 76, 181, and 194; 76, 118, 252, and 292; 76 and 274; 76, 102, 118, and 307; 12 and 76; 67, 95, and 226; 26 and 76; 22, 76, 319; 154 and 269; 76 and 238; 76, 238, 296, and 328; 7 and 76; 76 and 263; 59, 76, 306, and 316; or 280 and 340; relative to SEQ ID NO: 6. In select embodiments, the polypeptide comprises an amino acid sequence having an amino acid substitution at position 76, relative to SEQ ID NO:6.

In some embodiments, the polypeptide comprises an amino acid sequence having amino acid substitutions of: N2S, K67N or K67R, A95D, and V226E, relative to SEQ ID NO: 6. In some embodiments, the polypeptide comprises an amino acid sequence having amino acid substitutions of: E6D and N316K or N316D, relative to SEQ ID NO: 6. In some embodiments, the polypeptide comprises an amino acid sequence having amino acid substitutions of: N38S, A95D, E303D, relative to SEQ ID NO: 6. In some embodiments, the polypeptide comprises an amino acid sequence having amino acid substitutions of: K67N or K67R, A95D, and V226E, relative to SEQ ID NO: 6. In some embodiments, the polypeptide comprises an amino acid sequence having amino acid substitutions of: D44G or D44N and S76Y or K118N or K118R, relative to SEQ ID NO: 6. In some embodiments, the polypeptide comprises an amino acid sequence having amino acid substitutions of: D44G or D44N, I130V, N234H, E303D, relative to SEQ ID NO: 6. In some embodiments, the polypeptide comprises an amino acid sequence having amino acid substitutions of: K118N or K118R and A1201V, relative to SEQ ID NO: 6. In some embodiments, the polypeptide further comprises amino acid substitutions of: D44G or D44N or S76Y. In some embodiments, the polypeptide comprises an amino acid sequence having amino acid substitutions of: I130V, N234H, and E303D, relative to SEQ ID NO: 6. In some embodiments, the polypeptide comprises an amino acid sequence having amino acid substitutions of: R154K and E269K or E269D, relative to SEQ ID NO: 6. In some embodiments, the polypeptide comprises an amino acid sequence having amino acid substitutions of: K221N and D44G or D44N, relative to SEQ ID NO: 6. In some embodiments, the polypeptide comprises an amino acid sequence having amino acid substitutions of: F280L and S340L, relative to SEQ ID NO: 6. In some embodiments, the polypeptide comprises an amino acid sequence having amino acid substitutions of: D44N or D44G, S76Y, 1130V, N234H, and E303D, relative to SEQ ID NO: 6. In some embodiments, the polypeptide comprises an amino acid sequence having amino acid substitutions of: D44N or D44G, S76Y, K118R, and A1201V, relative to SEQ ID NO: 6. In select embodiments, the polypeptide comprises an amino acid sequence having an amino acid substitutions of: S76Y, relative to SEQ ID NO: 6. In select embodiments, the polypeptide comprises an amino acid sequence having an amino acid substitutions of: R197I and N314K, relative to SEQ ID NO: 6. In select embodiments, the polypeptide comprises an amino acid sequence having an amino acid substitutions of: S76Y, A181S, and V194M, relative to SEQ ID NO: 6. In select embodiments, the polypeptide comprises an amino acid sequence having an amino acid substitutions of: S76Y, K118R, H252R, and K292N, relative to SEQ ID NO: 6. In select embodiments, the polypeptide comprises an amino acid sequence having an amino acid substitutions of: S76Y and I274V, relative to SEQ ID NO: 6. In select embodiments, the polypeptide comprises an amino acid sequence having an amino acid substitutions of: S76Y, A102T, K118R, and V307G, relative to SEQ ID NO: 6. In select embodiments, the polypeptide comprises an amino acid sequence having an amino acid substitutions of: L12M and S76Y, relative to SEQ ID NO: 6. In select embodiments, the polypeptide comprises an amino acid sequence having an amino acid substitutions of: K67N, A95D, and V226E, relative to SEQ ID NO: 6. In select embodiments, the polypeptide comprises an amino acid sequence having an amino acid substitutions of: K26N and S76Y, relative to SEQ ID NO: 6. In select embodiments, the polypeptide comprises an amino acid sequence having an amino acid substitutions of: H22Y, S76Y, and D319N, relative to SEQ ID NO: 6. In select embodiments, the polypeptide comprises an amino acid sequence having an amino acid substitutions of: R154K and E269D, relative to SEQ ID NO: 6. In select embodiments, the polypeptide comprises an amino acid sequence having an amino acid substitutions of: S76Y and A238S, relative to SEQ ID NO: 6. In select embodiments, the polypeptide comprises an amino acid sequence having an amino acid substitutions of: S76Y and S263N, relative to SEQ ID NO: 6. In select embodiments, the polypeptide comprises an amino acid sequence having an amino acid substitutions of: S59T, S76Y, E306G, and N316D, relative to SEQ ID NO: 6.

Provided herein is a polypeptide having at least 70% (e.g., having at least 75%, at least 80%, at least 85%, at least 90%, at least 92%, at least 95%, at least 96%, at least 97%, at least 98%, or at least 99%) identity to SEQ ID NO: 7 and one or more amino acid substitutions at positions: 99, 133, 189, 265, 266, 336, and 343, relative to SEQ ID NO: 7. In some embodiments, the polypeptide comprises an amino acid sequence having one or more amino acid substitutions of: M99I, S189N, H265Q, A266V, L336F, and V343A, relative to SEQ ID NO: 7.

Provided herein is a polypeptide having at least 70% (e.g., having at least 75%, at least 80%, at least 85%, at least 90%, at least 92%, at least 95%, at least 96%, at least 97%, at least 98%, or at least 99%) identity to SEQ ID NO: 8 and one or more amino acid substitutions at positions: 119, 134, 155, 180, 183, 274, 319, 447, 454, 458, 461, 512, 538, and 580, relative to SEQ ID NO: 8. In some embodiments, the polypeptide comprises an amino acid sequence having one or more amino acid substitutions of: Y119H, N134R, N134Q, D155N, Q180R, D183N, R274L, N319D, V447I, A454S, E458G, D461N, A512T, D538K, and P580Q, relative to SEQ ID NO: 8.

Provided herein is a polypeptide having at least 70% (e.g., having at least 75%, at least 80%, at least 85%, at least 90%, at least 92%, at least 95%, at least 96%, at least 97%, at least 98%, or at least 99%) identity to SEQ ID NO: 9 and one or more amino acid substitutions at positions: 28, 82, 144, 151, 162, 182, 273, 327, 346, relative to SEQ ID NO: 9. In some embodiments, the polypeptide comprises an amino acid sequence having one or more amino acid substitutions of: R28K, A82T, K144E, C151R, N162S, K182E, D273G, A327D, and M346I, relative to SEQ ID NO: 9.

Provided herein is a polypeptide having at least 70% (e.g., having at least 75%, at least 80%, at least 85%, at least 90%, at least 92%, at least 95%, at least 96%, at least 97%, at least 98%, or at least 99%) identity to SEQ ID NO: 10 and one or more amino acid substitutions at positions: 21 and 90, relative to SEQ ID NO: 10. In some embodiments, the polypeptide comprises an amino acid sequence having one or more amino acid substitutions of: A21S and V90A, relative to SEQ ID NO: 10.

Provided herein is a polypeptide having at least 70% (e.g., having at least 75%, at least 80%, at least 85%, at least 90%, at least 92%, at least 95%, at least 96%, at least 97%, at least 98%, or at least 99%) identity to SEQ ID NO: 11 and one or more amino acid substitutions at positions: 2, 3, 7, 9, 11, 12, 14, 16, 20, 26, 29, 32, 34, 35, 40, 43, 45, 46, 54, 61, 64, 65, 70, 77, 101, 103, 105, 106, 108, 109, 111, 119, 120, 123, 126, 127, 130, 131, 148, 149, 151, 157, 159, 164, 166, 185, 194, 196, 203, 211, 217, 218, 219, 236, 242, 257, 267, 279, 283, 286, 288, 291, 293, 296, 303, 306, 313, 314, 316, 326, 331, 336, 347, 352, 361, 374, 377, 395, 396, 398, and 408, relative to SEQ ID NO: 11. In some embodiments, the polypeptide comprises an amino acid sequence having one or more amino acid substitutions of: A2T, F3S, P7R, A9S, A9G, A11G, F12I, D14N, S16Y, Y20H, S26N, F29S, S32N, E34K, G35V, G35S, G35D, I40S, E43D, H45P, E46K, A54S, R61W, V64M, Y65C, N70S, A77T, D101N, K103E, N105K, N105D, S106G, V108M, A109G, Y111N, L119M, R120S, R123S, A126T, E127G, V130M, D131N, Q148R, S149Y, H151Y, A157D, T159I, A164V, L166M, T185A, S194G, A196T, T203A, K211R, E217K, R218K, R218S, N219S, A236T, E242D, N257K, N267S, M279I, M279V, D283G, N286S, T288I, K291Q, I293V, D296N, S303I, S303G, K306N, S310Y, S310P, I313T, Y314F, A316T, E326G, T331I, A336V, A347T, A347S, T352S, Y361H, M374T, M374I, R377G, T395I, S396T, S396F, G398V, and A408V, relative to SEQ ID NO: 11. In some embodiments, the polypeptide comprises an amino acid sequence having one or more amino acid substitutions at positions: 105, 109, 131, 148, 279, and 310; or 9, 105, 109, 131, 148, 279, and 310, relative to SEQ ID NO: 11. In some embodiments, the polypeptide comprises an amino acid sequence having one or more amino acid substitutions of: N105K, A109G, D131N, Q148R, M279I, and S310Y or S310P, relative to SEQ ID NO: 11. In some embodiments, the polypeptide comprises an amino acid sequence having one or more amino acid substitutions of: A9S, N105K, A109G, D131N, A148R, M279I, and S310P, relative to SEQ ID NO: 11.

In some embodiments, the polypeptide comprises an amino acid sequence having one or more additions to SEQ ID NO: 11. In some embodiments, the polypeptide comprises an amino acid sequence having a C-terminal addition of at least one amino acid. In some embodiments, the polypeptide comprises an amino acid sequence having 410L. Provided herein is a polypeptide having at least 70% (e.g., having at least 75%, at least 80%, at least 85%, at least 90%, at least 92%, at least 95%, at least 96%, at least 97%, at least 98%, or at least 99%) identity to SEQ ID NO: 12 and one or more amino acid substitutions at positions: 4, 5, 6, 8, 9, 11, 12, 13, 16, 17, 20, 21, 24, 26, 28, 29, 34, 37, 38, 41, 49, 54, 59, 60, 63, 65, 67, 74, 77, 81, 88, 92, 93, 94, 96, 102, 105, 106, 108, 110, 121, 126, 128, 134, 138, 142, 147, 150, 151, 153, 156, 157, 160, 162, 165, 170, 171, 173, 174, 179, 181, 183, 185, 186, 187, 188, 191, 198, 201, 206, 207, 226, 228, 233, 236, 241, 249, 250, 256, 267, 268, 270, 275, 276, 277, 279, 283, 286, 289, 303, 305, 306, 310, 312, 314, 315, 316, 323, 326, 329, 349, 353, 355, 356, 357, 358, 361, 370, 372, 373, 376, 378, 382, 388, 391, 397, 399, 403, 405, 419, 421, 423, 424, 425, 427, 428, 430, 431, 432, 433, 449, 457, 473, 477, 480, 485, 487, 489, 494, 496, 497, 498, 500, 502, 509, 511, 515, 518, 519, 520, 540, 545, 550, 555, 557, 570, 571, 580, 583, 585, 590, 594, 603, 607, 608, 611, 617, 620, 624, 636, 639, 641, 642, 644, 646, 655, 658, 660, 663, 665, 668, 672, 673, 678, 682, 685, 688, and 695, relative to SEQ ID NO: 12. In some embodiments, the polypeptide comprises an amino acid sequence having one or more amino acid substitutions of: K4N, E5K, L6M, L6I, E8K, E8D, 19T, D11N, T12A, T13I, D16G, R17C, R17S, R20K, R20E, R21E, R21K, S24K, S24Q, S24R, Y26S, Y26H, A28S, A28D, M29I, G34D, A37S, V38M, V38G, I41V, R49L, D54G, K59R, K60N, K63N, A65T, A65V, K67E, K74E, K77E, W81C, K88R, K88E, 192T, R93E, R93K, V94M, K96N, E102D, E102G, T105A, L106M, S108P, V110A, G121S, S126P, K128R, L134M, Y138S, Q142H, W147L, K150N, V151M, V151L, A153T, S156R, S156G, D157N, K160R, K160E, A162T, S165N, S165G, V170E, K171E, F173V, K174N, K174R, T179A, K181T, S183N, P185T, E186K, E186D, E187K, A188S, A188V, D191Y, D191E, R198H, R198C, R198S, R201K, D206G, G207D, A226T, I228V, R233K, N236T, R241E, A249S, A250S, I256T, S267G, S267N, K268N, H270P, S275N, S275G, R276G, A277D, A277S, A277T, K279N, G283D, V286G, V289M, G303D, I305T, F306S, A310D, A310T, A312G, A312D, A312T, K314N, Q315R, R316G, N323S, E326A, E326K, N329S, G349D, E353D, L355M, L355R, E356G, E356D, S357P, A358V, R361S, P370T, N372K, E373D, S376F, T378I, F382L, M388V, G391S, R397K, A399S, K403N, M405I, L419P, D421N, K423R, H424N, H424R, V425L, I427V, E428K, D430A, D431G, E432D, H433N, A449T, G457D, R473K, E477D, G480D, F485L, S487R, S487G, S489N, N494D, S496N, A497S, V498G, K500N, K502N, Q509R, A511T, A511E, R515S, R518S, P519T, G520D, G520V, Y540C, Q545H, K550N, K555E, H557Q, P570S, E571D, C580R, S583R, E585K, E585G, E590D, R594K, M6031, H607N, H607L, K608R, D611N, L617P, N620S, K624N, T636P, M639V, N641S, V642G, S644N, S644G, E646D, A655V, V658M, K660N, T663A, T665I, R668S, I672V, G673V, S678R, M682L, A685V, A685D, K688N, and V695M, relative to SEQ ID NO: 12.

In some embodiments, the polypeptide comprises an amino acid sequence having amino acid substitutions at positions: 134, 179, 185, 540, 555, 624, and 646; 138, 250, 275, and 421; 303, 405, 520, and 590; 134, 179, 185, 540, 555, and 646, 4 and 49; 4 and 388; 4 and 571; 4, 162, and 480; 4 and 315; 5 and 316; 17 and 156; 38, 108, 497 and 583; 59, 157 and 644; 96, 305, 550 and 642; 106, 160 and 228; 312, 424, 449 and 457; or 376 and 611, relative to SEQ ID NO: 12. In some embodiments, the polypeptide comprises an amino acid sequence having amino acid substitutions of: L134M, T179A, P185T, Y540C, K555E, K624N, and E646D, relative to SEQ ID NO: 12. In some embodiments, the polypeptide comprises an amino acid sequence having amino acid substitutions of: Y138S, A250S, S275N, and D421N, relative to SEQ ID NO: 12. In some embodiments, the polypeptide comprises an amino acid sequence having amino acid substitutions of: L134M, T179A, P185T, Y540C, K555E, and E646D, relative to SEQ ID NO: 12. In some embodiments, the polypeptide comprises an amino acid sequence having amino acid substitutions of: G303D, M405I, G520D, and E590D, relative to SEQ ID NO: 12.

Provided herein is a polypeptide having at least 70% (e.g., having at least 75%, at least 80%, at least 85%, at least 90%, at least 92%, at least 95%, at least 96%, at least 97%, at least 98%, or at least 99%) identity to SEQ ID NO: 13 and one or more amino acid substitutions at positions: 5, 10, 11, 26, 30, 35, 40, 42, 45, 46, 47, 58, 61, 65, 71, 72, 75, 77, 78, 80, 82, 83, 94, 98, 113, 115, 116, 117, 121, 128, 133, 138, 146, 148, 161, 171, 175, 177, 182, 184, 191, 193, 201, 203, 211, 212, 219, 225, 226, 232, 233, 235, 236, 237, 238, 240, 250, 274, 282, 286, 292, 295, 304, 307, 309, 312, 313, 315, 316, 317, 318, 320, 321, 322, 323, 328, 340, 343, 344, 345, 347, 348, 349, and 350, relative to SEQ ID NO: 13. In some embodiments, the polypeptide comprises an amino acid sequence having one or more amino acid substitutions of: N5K, N5T, DION, R11K, D26N, V30E, D35N, R40L, P42A, G45S, G45V, F46V, T47R, T47S, N58T, P61L, T65I, T71I, T71R, T71D, L72M, C75S, V77A, P78L, N80T, E82D, H83Y, H83N, A94S, V98M, E113D, C121F, A128S, A155S, E116D, T117I, R133K, G138V, N146D, G148V, C161R, A171V, A171S, K175T, A177V, K182E, L184M, I191V, S193A, S193F, F201S, S203N, E211K, A212V, Y219R, N225S, N225T, D226Y, E232K, E232Q, A233N, A233S, A233K, K235R, Q236R, Q236S, F237L, V238Q, V238M, A240T, A240V, S250A, R274G, A282V, I286N, I286T, I286F, P292S, S295N, K304R, E307D, Y309C, A312V, L313M, N315K, N315T, N315S, C316G, I317V, T318A, T318P, K320R, N321D, E322K, K323N, I328T, M340I, K343E, K343R, K344E, K344R, A345T, A345D, A345S, A345Y, A345R, A345K, A345E, A345G, A347K, A347S, A347D, K348N, K349R, A350K, A350D, A350V, and A350T, relative to SEQ ID NO: 13.

In some embodiments, the polypeptide comprises an amino acid sequence having amino acid substitutions at positions: 30, 46, 240, 304, and 316; 30, 46, 240, and 316; 42 and 318; 184, 240, 315, and 345; 211 and 274; 237 and 237; 286 and 350; 317 and 347; 171, 286, and 315; or 328 and 350, relative to SEQ ID NO: 13. In some embodiments, the polypeptide comprises an amino acid sequence having amino acid substitutions of: V30E, F46V, A240T or A240V, K304R, and C316G, relative to SEQ ID NO: 13. In some embodiments, the polypeptide comprises an amino acid sequence having amino acid substitutions of: V30E, F46V, A240T or A240V, and C316G, relative to SEQ ID NO: 13. In some embodiments, the polypeptide comprises an amino acid sequence having amino acid substitutions of: L184M, A240T or A240V, N315K, and A345T, relative to SEQ ID NO: 13. In some embodiments, the polypeptide comprises an amino acid sequence having amino acid substitutions of: 1286N and A350D, relative to SEQ ID NO: 13. In some embodiments, the polypeptide comprises an amino acid sequence having amino acid substitutions of: A171S, I286F, and N315S, relative to SEQ ID NO: 13.

Provided herein is a polypeptide having at least 70% (e.g., having at least 75%, at least 80%, at least 85%, at least 90%, at least 92%, at least 95%, at least 96%, at least 97%, at least 98%, or at least 99%) identity to SEQ ID NO: 13 and at least one amino acid substitution with a positively charged amino acid. In some embodiments, the positively charged amino acid is arginine. In some embodiments, the at least one amino acid substitution is at position 2, 5, 6, 7, 8, 9, 10, 12, 38, 39, 40, 41, 42, 43, 44, 45, 46, 47, 48, 64, 65, 66, 67, 68, 69, 70, 222, 224, 225, 227, 228, 229, 231, 232, 233, 234, 235, 255, 256, 257, 258, 277, 286, 287, 337, 338, 339, 340, 345, 346, 347, 348, 349, 350, or a combination thereof, relative to SEQ ID NO: 13. In some embodiments, the at least one amino acid substitution is at positions: 346 and 348; 346, 348 and 349; 346, 348, 349, an 350; 350 and 351; 350, 351, and 352; 350, 351, 352, and 353; 235 and 227; 235 and 345; 235 and 346; 235 and 347; 235 and 348; 235 and 349; 235 and 350; 235, 227, and 349; 5, 235 and 346; 5, 235 and 348; 5, 235 and 349; 227, 235, and 346; or 227, 235, and 348, relative to SEQ ID NO: 13.

Provided herein is a polypeptide having at least 70% (e.g., having at least 75%, at least 80%, at least 85%, at least 90%, at least 92%, at least 95%, at least 96%, at least 97%, at least 98%, or at least 99%) identity to SEQ ID NO: 14 and one or more amino acid substitutions at positions: 2, 9, 13, 14, 15, 34, 38, 42, 46, 50, 59, 60, 73, 75, 77, 82, 83, 85, 86, 97, 110, 115, 120, 124, 130, 132, 134, 140, 143, 145, 156, 159, 162, 164, 177, 199, 232, and 270, relative to SEQ ID NO: 14. In some embodiments, the polypeptide comprises an amino acid sequence having one or more amino acid substitutions of: Q2K, H9L, K13E, Q14K, A15G, K34N, E38K, V42I, A46D, S50I, V59G, Y60H, A73S, A73T, F75L, D77G, G82S, F83L, F83V, F83C, K85E, V86I, E97, I110S, I110L, S115R, K120N, K124R, G130D, D132E, N134T, A140T, E143K, D145G, S156I, E159K, I162V, H164Y, H164F, Y177C, S199I, S232L, and L270S, relative to SEQ ID NO: 14.

In some embodiments, the polypeptide comprises an amino acid sequence having one or more amino acid substitutions at positions: 82, 110, 115, 164, and 199, relative to SEQ ID NO: 14. In some embodiments, the polypeptide comprises an amino acid sequence having one or more amino acid substitutions of: G82S, I110S, S115R, H164Y or H164F, and S199I, relative to SEQ ID NO: 14. In some embodiments, the polypeptide comprises an amino acid sequence having one or more amino acid substitutions at positions: 110, 115, 164, 199, and 124, relative to SEQ ID NO: 14. In some embodiments, the polypeptide comprises an amino acid sequence having one or more amino acid substitutions of: I110S, S115R, K124R, H164Y or H164F, and S199I, relative to SEQ ID NO: 14. In some embodiments, the polypeptide comprises an amino acid sequence having one or more amino acid substitutions at positions: 82, 110, 115, 124, 164, and 199, relative to SEQ ID NO: 14. In some embodiments, the polypeptide comprises an amino acid sequence having one or more amino acid substitutions of: G82S, I110S, S115R, K124R, H164Y, and S199I, relative to SEQ ID NO: 14. In some embodiments, the polypeptide comprises an amino acid sequence having one or more amino acid substitutions at positions: 110, 115, and 164, relative to SEQ ID NO: 14. In some embodiments, the polypeptide comprises an amino acid sequence having one or more amino acid substitutions of: I110S, S115R, and H164F, relative to SEQ ID NO: 14. In some embodiments, the polypeptide comprises an amino acid sequence having one or more amino acid substitutions at positions: 110, 115, 164, and 199, relative to SEQ ID NO: 14. In some embodiments, the polypeptide comprises an amino acid sequence having one or more amino acid substitutions of: I110S, S115R, H164F, and S199I, relative to SEQ ID NO: 14. In some embodiments, the polypeptide comprises an amino acid sequence having one or more amino acid substitutions at positions: 110, 115, 164, 199, and 124, relative to SEQ ID NO: 14. In some embodiments, the polypeptide comprises an amino acid sequence having one or more amino acid substitutions of: I110S, S115R, H164F, S199I, and K124R, relative to SEQ ID NO: 14.

The polypeptides may be part of a fusion protein comprising a first amino acid sequence for a polypeptide disclosed herein and a second amino acid sequence. The term “fusion protein” as used herein refers to a polypeptide which comprises at least two different proteins or at least two protein domains from two different proteins. The fusion protein is not limited by orientation of the at least two different proteins. For example, the arrangement of the first protein in the fusion protein may be N-terminal or C-terminal to the second protein.

The fusion protein may comprise a linker polypeptide between the first amino acid sequence and the second amino acid sequence. The linker polypeptide may have any of a variety of amino acid sequences. Proteins can be joined by a linker polypeptide, generally of a flexible nature, although other chemical linkages are not excluded. Suitable linkers include polypeptides of between 4 amino acids and 40 amino acids in length, or between 4 amino acids and 25 amino acids in length. The linking peptides may have virtually any amino acid sequence, bearing in mind that the preferred linkers will have a sequence that results in a generally flexible peptide. Small amino acids, such as glycine and alanine, are useful in creating a flexible peptide linker. A variety of different linkers are considered suitable for use, including but not limited to, glycine-serine polymers, glycine-alanine polymers, and alanine-serine polymers.

In some embodiments, the second amino acid sequence is a sequence of another protein or protein domain. For example, a polypeptide as disclosed herein may be fused to another protein or protein domain that provides for tagging or visualization (e.g., GFP) or for entry into a cell (e.g., protein transduction domains or PTDs, also known as a CPP, a cell penetrating peptide) or cellular compartment (e.g., the nucleus with a nuclear localization sequence as described elsewhere herein), or additional functionality (e.g., transcriptional activator/repressor or nucleic acid or protein binding activity). In some embodiments, the second amino acid sequence is an amino acid sequence disclosed herein. Thus, fusion proteins comprising sequences for two of the disclosed polypeptides are encompassed by embodiments of the disclosure.

Accordingly, provided herein are polypeptides (e.g., single polypeptide chains) comprising two or more amino acid sequences having at least 70% (e.g., having at least 75%, at least 80%, at least 85%, at least 90%, at least 92%, at least 95%, at least 96%, at least 97%, at least 98%, or at least 99%) identity to any of SEQ ID NOs: 1-14. In some embodiments, the fusion polypeptide comprises a first amino acid sequence from one of the disclosed Cas or transposase proteins having at least 70% (e.g., having at least 75%, at least 80%, at least 85%, at least 90%, at least 92%, at least 95%, at least 96%, at least 97%, at least 98%, or at least 99%) identity to any of SEQ ID NOs: 1-14 with one or more amino acid substitutions, deletions, or additions relative to any of SEQ ID NOs: 1-14. In some embodiments, the fusion polypeptide further comprises a second amino acid sequence from one of the disclosed Cas or transposase proteins having at least 70% (e.g., having at least 75%, at least 80%, at least 85%, at least 90%, at least 92%, at least 95%, at least 96%, at least 97%, at least 98%, or at least 99%) identity to any of SEQ ID NOs: 1-14 with one or more amino acid substitutions, deletions, or additions relative to any of SEQ ID NOs: 1-14. For example, the fusion polypeptide may comprise two or more of the disclosed transposase proteins (e.g., a first sequence having a sequence encoding a TnsA protein of at least 70% (e.g., having at least 75%, at least 80%, at least 85%, at least 90%, at least 92%, at least 95%, at least 96%, at least 97%, at least 98%, or at least 99%) identity to SEQ ID NO: 1 and a second sequence having a sequence encoding a TnsB protein of at least 70% (e.g., having at least 75%, at least 80%, at least 85%, at least 90%, at least 92%, at least 95%, at least 96%, at least 97%, at least 98%, or at least 99%) identity to SEQ ID NO: 2).

As such, the polypeptide may comprise a first amino acid sequence having at least 70% (e.g., having at least 75%, at least 80%, at least 85%, at least 90%, at least 92%, at least 95%, at least 96%, at least 97%, at least 98%, or at least 99%) identity to any of SEQ ID NOs: 1-14 and a second amino acid sequence having at least 70% (e.g., having at least 75%, at least 80%, at least 85%, at least 90%, at least 92%, at least 95%, at least 96%, at least 97%, at least 98%, or at least 99%) identity to any of SEQ ID NOs: 1-14.

In some embodiments, the polypeptide comprises a first amino acid sequence having a sequence encoding a TnsA protein of at least 70% (e.g., having at least 75%, at least 80%, at least 85%, at least 90%, at least 92%, at least 95%, at least 96%, at least 97%, at least 98%, or at least 99%) identity to SEQ ID NO: 1 with one or more amino acid substitutions, deletions, or additions relative to SEQ ID NO: 1, and a second amino acid sequence having a sequence encoding a TnsB protein of at least 70% (e.g., having at least 75%, at least 80%, at least 85%, at least 90%, at least 92%, at least 95%, at least 96%, at least 97%, at least 98%, or at least 99%) identity to SEQ ID NO: 2 with one or more amino acid substitutions, deletions, or additions relative to SEQ ID NO: 2.

In some embodiments, the first amino acid sequence comprises one or more amino acid substitutions at positions: 2, 3, 5, 28, 57, 77, 80, 107, 110, 116, 122, 142, 155, 161, 166, 173, 177, 185, 211, 216, 227, and 230, relative to SEQ ID NO: 1. In some embodiments, the first amino acid sequence comprises an amino acid sequence having one or more amino acid substitutions of: A2T, T3I, L5S, T28A, A57T, F77L, Y80D, K107M, K107R, Y110C, Y110D, D116G, E122A, D142E, M155I, K161R, N166D, K173E, Y177N, Y177D, C185R, D211Y, K216E, A227P, G230D, and G230S, relative to SEQ ID NO: 1.

In some embodiments, the first amino acid sequence comprises amino acid substitutions at positions: 2 and 230; 107 and 166; 107, 166, and one or both of: 2 and 227; 211 and 110 or 142; 110, 155 and 230; 122 and 155; 155 and 177, relative to SEQ ID NO: 1. In some embodiments, the first amino acid sequence comprises amino acid substitutions of: A2T, and G230D or G230S, relative to SEQ ID NO: 1. In some embodiments, the first amino acid sequence comprises amino acid substitutions of: K107M and N166D, relative to SEQ ID NO: 1. In some embodiments, the first amino acid sequence comprises amino acid substitutions of: A2T and/or Y177N or Y177D, relative to SEQ ID NO: 1. In some embodiments, the first amino acid sequence comprises amino acid substitutions of: D211Y and Y110C, Y110D, or D142E, relative to SEQ ID NO: 1. In some embodiments, the first amino acid sequence comprises amino acid substitutions of: Y110C or Y110D, M155I, and G230D or G230S, relative to SEQ ID NO: 1. In some embodiments, the first amino acid sequence comprises amino acid substitutions of: E122A and M155I, relative to SEQ ID NO: 1. In some embodiments, the first amino acid sequence comprises amino acid substitutions of: M155I and Y177N or Y177D, relative to SEQ ID NO: 1.

In some embodiments, the second amino acid comprises one or more amino acid substitutions at positions: 2, 5, 22, 24, 25, 29, 75, 141, 199, 215, 319, 347, 364, 370, 383, 439, 454, 458, 485, 509, 533, 538, 565, 581, 586, 595, 596, 597, and 600, relative to SEQ ID NO: 2. In some embodiments, the second amino acid comprises one or more amino acid substitutions of: A2T, A2S, G5R, S22P, E24D, L25I, A29S, P75T, I141T, V199I, S215R, D319V, Y347F, S364N, E370K, N383D, V439A, E454D, E454G, S458N, V485F, R509G, D533A, A538V, H565Y, A581T, H586L, N595K, D596N, D597N, D597Y, and I600V, relative to SEQ ID NO: 2. In some embodiments, the second amino acid comprises amino acid substitutions at positions: 2 and 597; 24 and 25; 24, 25, 458, 509, 565, and 600; 75 and 597; 141, 454, 533 and 595; 581, 370, and 454; 370 and 581; 370 and 454; 458 and 509; 458, 509 and 565; 458, 509, 565, and 600; 565, 586, and 596; or 565, 509, 458, 600 and at least one of 24, 25, 29, 215, 319, 364, 383, and 586, relative to SEQ ID NO: 2.

In some embodiments, the second amino acid comprises amino acid substitutions of: A2T or A2S, and D597N or D597Y, relative to SEQ ID NO: 2. In some embodiments, the second amino acid comprises amino acid substitutions of: E24D and L25I, relative to SEQ ID NO: 2. In some embodiments, the second amino acid comprises amino acid substitutions of: E24D and L25I, S458N, R509G, H565Y, and I600V, relative to SEQ ID NO: 2. In some embodiments, the second amino acid comprises amino acid substitutions of: P75T and D597N or D597Y, relative to SEQ ID NO: 2. In some embodiments, the second amino acid comprises substitutions of: E454D or E454G, D533A, N595K, relative to SEQ ID NO: 2. In some embodiments, the second amino acid comprises amino acid substitutions of: E370K, E454D or E454G, and A581T, relative to SEQ ID NO: 2. In some embodiments, the second amino acid comprises amino acid substitutions of: E370K, and A581T, E454D, or E454G, relative to SEQ ID NO: 2. In some embodiments, the second amino acid comprises amino acid substitutions of: S458N and R509G, relative to SEQ ID NO: 2. In some embodiments, the second amino acid comprises amino acid substitutions of H565Y and/or I600V. In some embodiments, the second amino acid comprises amino acid substitutions of: H565Y, H586L, and D596N, relative to SEQ ID NO: 2. In some embodiments, the second amino acid comprises amino acid substitutions of: H565Y, R509G, S458N, I600V and at least one of E24D, L25I, A29S, S215R, D319V, S364N, N383D, and H586L, relative to SEQ ID NO: 2.

In some embodiments, the polypeptide comprises a first amino acid sequence having a sequence encoding a TnsA protein of at least 70% (e.g., having at least 75%, at least 80%, at least 85%, at least 90%, at least 92%, at least 95%, at least 96%, at least 97%, at least 98%, or at least 99%) identity to SEQ ID NO: 4 with one or more amino acid substitutions, deletions, or additions relative to SEQ ID NO: 4, and a second amino acid sequence having a sequence encoding a TnsB protein of at least 70% (e.g., having at least 75%, at least 80%, at least 85%, at least 90%, at least 92%, at least 95%, at least 96%, at least 97%, at least 98%, or at least 99%) identity to SEQ ID NO: 5 with one or more amino acid substitutions, deletions, or additions relative to SEQ ID NO: 5.

In some embodiments, the first amino acid sequence comprises one or more amino acid substitutions at positions: 4, 5, 9, 10, 12, 21, 23, 25, 26, 31, 32, 34, 35, 37, 41, 45, 47, 48, 51, 52, 55, 60, 61, 65, 67, 69, 72, 75, 79, 80, 82, 87, 88, 90, 91, 93, 94, 96, 98, 99, 100, 103, 106, 108, 113, 116, 125, 126, 128, 129, 135, 139, 143, 146, 147, 149, 153, 154, 156, 158, 159, 160, 162, 164, 166, 167, 168, 169, 170, 177, 179, 180, 182, 183, 185, 187, 188, 190, 191, 192, 193, 195, 196, 200, 204, 207, and 208, relative to SEQ ID NO: 4. In some embodiments, the first amino acid sequence comprises one or more amino acid substitutions of: R4K, N5K, P9S, A10P, N12D, T21I, V23M, S25N, S25R, V26M, V26G, S31N, S321, E34A, F35L, A37D, H41L, D45N, I47V, E48G, G51V, S52I, E55K, E55D, E60K, F61L, S65T, S65A, P67T, P67L, P67S, P67H, T69A, A72V, A72D, S75I, S75R, S75T, K79E, T80P, K82E, K87R, P88L, P88T, P88A, S90F, K91N, K91E, A93T, A93S, S94N, L96P, R98Q, A99D, A99V, E100K, A103T, A106T, S108A, I113F, V116F, V116I, V125M, V125A, N126T, 1128V, 1128L, L129P, L135M, S139N, S139G, G143V, G143C, G146D, G146S, I147V, K149E, K149T, K149R, S153I, S153R, S153N, F154C, H156R, H156L, S158N, S158R, G159V, V160A, K162R, N164D, I166L, S167I, S168I, S168R, S168N, Q169R, V170M, V170G, V170L, T177I, T177A, S179R, F180C, F180L, F182C, F182L, G183S, M185I, K187R, G188D, V190I, K191N, A192S, D193N, G195V, G195D, G195S, C196W, T200A, T204I, A207V, A207T, and T208I, relative to SEQ ID NO: 4.

In some embodiments, the first amino acid sequence comprises amino acid substitutions at positions: 108 and 47 or 208; 170 and 207; 88 and 147; 47, 88 and 147; 88, 147, 170, and 182; 88, 147, 170, 182, and 51 or 180; 88, 147, and 154; 88, 128, 147, 170, and 182; or 170, 207, and 108, relative to SEQ ID NO: 4. In some embodiments, the first amino acid sequence comprises amino acid substitutions of: S108A, and I47V or T208I, relative to SEQ ID NO: 4. In some embodiments, the first amino acid sequence comprises amino acid substitutions of: V170M, and A207V or A207T, relative to SEQ ID NO: 4. In some embodiments, the first amino acid sequence comprises amino acid substitutions of: S108A, V170M, and A207V or A207T, relative to SEQ ID NO: 4. In some embodiments, the first amino acid sequence comprises amino acid substitutions of: P88T, I147V, V170L, and F182L, relative to SEQ ID NO: 4. In some embodiments, the first amino acid sequence comprises amino acid substitutions of: P88T, I147V, V170L, F182L and G51V or F180L, relative to SEQ ID NO: 4. In some embodiments, the first amino acid sequence comprises amino acid substitutions of: P88T, I147V, and F154C, relative to SEQ ID NO: 4.

In some embodiments, the second amino acid sequence comprises one or more amino acid substitutions at positions: 1, 2, 4, 5, 8, 9, 10, 11, 12, 13, 14, 15, 16, 17, 19, 20, 21, 22, 23, 24, 25, 26, 27, 28, 29, 30, 32, 33, 36, 37, 39, 40, 41, 42, 43, 44, 45, 49, 52, 55, 56, 58, 60, 62, 63, 67, 71, 74, 76, 78, 79, 80, 81, 82, 83, 84, 85, 86, 87, 88, 89, 91, 92, 95, 97, 100, 101, 104, 106, 110, 112, 113, 115, 117, 119, 120, 124, 125, 127, 129, 130, 131, 134, 139, 142, 144, 145, 146, 147, 149, 150, 155, 156, 157, 158, 159, 163, 164, 165, 167, 169, 173, 174, 176, 181, 182, 186, 187, 190, 195, 197, 198, 205, 208, 209, 211, 215, 218, 223, 226, 227, 231, 232, 235, 239, 246, 248, 250, 259, 260, 261, 262, 263, 267, 269, 273, 274, 277, 278, 280, 281, 282, 283, 285, 287, 288, 290, 295, 298, 302, 303, 307, 313, 316, 317, 320, 323, 325, 331, 332, 339, 345, 348, 349, 352, 353, 354, 356, 361, 362, 363, 364, 365, 366, 367, 369, 370, 371, 372, 373, 375, 376, 380, 383, 385, 386, 389, 390, 392, 396, 397, 399, 402, 403, 404, 407, 408, 410, 411, 412, 413, 414, 446, 448, 450, 452, 456, 459, 460, 463, 464, 470, 472, 473, 494, 495, 498, 501, 502, 504, 505, 506, 508, 509, 510, 512, 513, 514, 517, 520, 521, 522, 525, 526, 527, 530, 531, 532, 533, 535, 537, 538, 540, 541, 542, 543, 544, 545, 546, 547, 548, 549, 550, 551, 552, 553, 554, 556, 557, 558, 559, 560, 561, 562, 563, 564, 565, 567, 568, 569, 570, 571, 574, 575, 576, 580, 582, 583, 584, 585, 586, 587, 588, 589, 590, 591, 592, 593, 594, 595, 596, 597, 599, 600, 601, 602, 603, 604, 606, 607, 608, 611, 613, 618, 620, and 656, relative to SEQ ID NO: 5.

In some embodiments, the second amino acid sequence comprises one or more amino acid substitutions of: M1V, M1I, M1L, T2I, T2A, F4L, F5L, F8L, F8V, F8S, D9N, E10K, E10D, S11I, S11R, S11G, L12P, V13M, V13G, V13E, V13L, P14L, L15Q, K16N, K16R, P17T, P17L, P17S, T19I, T19S, T19A, T19P, P20S, P20L, T21A, Q22R, Y23H, V24M, K25R, L26M, D27A, D27G, D28N, D28Y, A29T, A29V, N30K, I32F, I32S, Q33H, L36M, D37A, D37Y, F39L, S40P, D41E, T42I, T42K, T42A, F43L, F43S, F43V, K44N, N45D, N45S, Q49R, K52Q, S55A, T56A, D58E, K60Q, S62T, R63K, R63G, Q67R, Q67H, Q67K, D71Y, K74R, E76K, F78C, K79R, G80V, G80D, G81S, G81V, G81D, D82N, V83G, V83M, V83A, V84A, V84G, R85G, R85K, P86L, N87S, R89C, V91G, V91A, A92V, A92T, R95K, K97R, E100D, S101A, D104V, A106D, A106T, D110N, N112H, H113Y, M115R, N117Y, T119A, N120D, N120K, N120S, G124V, D125N, D125E, K127R, F129L, D130N, K131M, E134D, E134G, A139S, A139T, P142S, 1144V, A145S, A145T, T146A, A147V, Q149R, Y150H, 1155L, V156A, V156L, V156M, K157V, E158A, N159S, V163G, E164A, E164G, E164D, G165D, I167V, I169L, I169T, N173S, N173H, N173T, A174S, A174T, N176D, A181S, I182L, I182V, I182T, A186E, A186T, V187G, V187A, A190T, A190S, F195S, A197P, D198G, D198N, A205S, V208M, P209T, T211I, E215D, E218D, P223S, P223H, L226V, I227V, D231N, E232K, I235V, I235T, R239G, I246V, V248E, V248M, S250I, S259N, Y260C, K261R, S262N, P263L, S267N, A269V, T273I, T273N, H274Y, K277N, K277R, P278S, S280T, L281M, D282E, D282N, A283T, A283S, N285S, E287D, L288M, N290K, F295S, F298I, F298S, V302I, V303M, A307S, N313S, H316R, A317V, S320N, S320R, I323L, I325V, R331K, K332E, I339V, V345L, V345M, E348K, Y349H, Y349D, Y349N, Y349C, P352S, P352T, E353Q, E353D, L354M, G356S, N361D, I362V, I362T, L363P, L363T, L363M, E364G, K365R, E366G, E367G, K369N, K369E, K369M, P370S, E371K, V372M, D373G, I375V, M376I, T380P, T380A, E383K, E383D, F385L, H386Y, I389V, A390V, A390I, V3921, D396N, D396G, D396K, S397P, S399N, S399G, T402I, R403G, R403I, R403K, R403S, I404T, I404V, K407R, K407E, R408K, Q410K, Q410H, Q410R, Q411H, G412V, F413L, D414N, A415V, A415T, Y416C, M421I, N422K, E423K, E423D, E424A, E425K, E426D, T427A, T427S, R428K, F429L, S430A, M431L, R434H, R434C, R434S, I435V, D437G, D437N, T440S, T440I, R443C, G445S, F446L, F446I, Y448C, E450D, E450G, M452I, T456P, T456A, T456I, A459T, D460N, K463N, H464N, H464R, H464S, E470K, V472M, V472A, K473D, K473N, E494D, E494G, S495A, E498A, E498K, C501Y, T502I, T502S, P504S, P504L, T505A, G506Y, G506D, G506L, G506S, T508A, D509E, D509Y, C510Y, S512N, I513L, I513V, I513F, Y514H, K517M, K517N, K517Q, K520R, K521N, I522T, I522V, I522F, E525K, V526E, V526M, I527V, S530N, S530R, K531T, D532G, D532Y, S533Y, G535D, A537T, K538R, K538N, R540K, R540G, M541L, A542T, I543L, H544R, E545A, R546G, R546K, V547M, K548Q, K548R, Q549K, Q549R, E550A, Q551K, E552D, E552K, V553I, F554V, E556K, E556G, S557A, K558R, T559P, T559I, T559A, K560R, A561T, A561G, K562R, K562N, I563L, T564I, A565S, A565V, K567R, K568N, K568R, Q569K, Q569L, Q569R, A570V, Q571R, D574N, V575M, V575A, S576R, T580I, T580A, T582I, T582S, I583V, K584R, V585M, S586P, S586A, S586F, E587A, E588K, E588G, E588D, S589I, S589R, S589N, A590S, A590T, A591V, P592L, V593M, V593A, Q594L, K595R, K595N, H596Y, H596L, H596P, I597T, I597V, N599H, D600L, D600N, D600G, D600V, N601S, N601K, S602A, S602P, S602Y, D603A, D603V, D604G, D604Y, D604N, D606A, D606V, D606Y, D607Y, D607E, D608N, A611T, E613D, R618I, T620P, and A656V, relative to SEQ ID NO: 5.

In some embodiments, the second amino acid sequence comprises amino acid substitutions at positions: 4, 23 and 590; 19, 169, and 549; 43 and 415; 80 and 593; 80, 144, 593, and 606; 1, 42, 80, 593, and 606; 42, 80, 593, and 606; 156 and 604; 283, 349, and 365; 283, 349, 365, 396, and 594; 283, 349, 365, 396, 594, 596, and 131; 352 and 390; 390, 396, and 594; 396 and 594; 456 and 502; 464 and 502; 464 and 17; 17, 235, 464, and 596; 235, 352, 396, 456, and 606; 415, 456, and 502; 456, 502, and 549; 169, 456, 502, and 549; 80, 456, 502, 593, and 606; 1, 42, 80, 456, 502, 593, and 606; 80, 144, 456, 502, 593, and 606; 19, 169, 456, 502 and 549; 43, 415, 456, and 502; 352, 390, 396, and 594; 352, 390, and 396; 283, 349, 396, and 594; 11, 55, 120, 362, 584, 600, and 604; 43, 84, 144, 349, and 517; 164 and 165; 164 and 173; 362 and 446; 352, 390, 396, 549, and 594; 352, 390, 396, 464, 549, and 594; 43, 349, 352, 390, 396, 464, 549, and 594; 43, 349, 352, 390, 396, 464, 549, 594 and one or more positions selected from 63, 145, 174, 182, 208, 410, 427, 456, 504, and 526; 43, 352, 390, 396, 464, 549, and 594; 43, 349, 352, 390, 396, 464, 549, 594, 410, 526, 415, and 502; 43, 349, 352, 390, 396, 464, 549, 594, 410, 526, 415, 502, and 21; 43, 349, 352, 390, 396, 464, 549, 594, 410, 526, 415, 502, and 67; 43, 349, 352, 390, 396, 464, 549, 594, 410, 526, 415, 502, 21, and 67; 43, 349, 352, 390, 396, 464, 549, 594, 410, 526, 174, 208, 427, 456, and 504; 43, 349, 352, 390, 396, 464, 549, 594, 410, 526, 415, 502, and 139; 43, 349, 352, 390, 396, 464, 549, 594, 410, 526, 415, 502, 339, and 446; 43, 349, 352, 390, 396, 464, 549, 594, 410, 526, 19, 460, 569, and 596; 43, 349, 352, 390, 396, 464, 549, 594, 410, 526, 460, 586, 588, and 608; 43, 349, 352, 390, 396, 464, 549, 594, 410, 526, and 460; 352, 390, 396, 549, 586, and 594; 63, 158, 352, 390, 396, 549, 586, and 594; 164, 165, 352, 363, 390, 396, 410, 549, 586, and 594; 164, 173, 352, 390, 396, 549, 586, and 594; 83, 352, 390, 396, 549, 586, and 594; 8, 43, 174, 349, 352, 390, 396, 427, 464, 549, and 594; or 283, 349, 365, 396, 594, 596, and 131, relative to SEQ ID NO: 5.

In select embodiments, the second amino acid sequence comprises amino acid substitutions at positions: 352, 390, 396, 594, or any combination thereof, relative to SEQ ID NO: 5.

In select embodiments, the second amino acid comprises amino acid substitutions at positions: F43, Y349, P352, A390, D396, H464, Q549, Q594, and T456; F43, Y349, P352, A390, D396, H464, Q549, Q594, T456, and V526; F43, Y349, P352, A390, D396, H464, Q549, Q594, and P504; F43, Y349, P352, A390, D396, H464, Q549, Q594, and V526; F43, Y349, P352, A390, D396, H464, Q549, Q594, Q410, and V526; F43, Y349, P352, A390, D396, H464, Q549, Q594, A174, and T427; F43, Y349, P352, A390, D396, H464, Q549, Q594, and V208; F43, Y349, P352, A390, D396, H464, Q549, Q594, R63, A145, 1182, and V526; F43, Y349, P352, A390, D396, H464, Q549, Q594, Q410, V526, A415, and T502; F43, Y349, P352, A390, D396, H464, Q549, Q594, Q410, V526, A415, T502, and T21; F43, Y349, P352, A390, D396, H464, Q549, Q594, Q410, V526, A415, T502, and Q67; F43, Y349, P352, A390, D396, H464, Q549, Q594, Q410, V526, A415, T502, T21, and Q67; F43, Y349, P352, A390, D396, H464, Q549, Q594, Q410, V526, A174, V208, T427, T456, and P504; F43, Y349, P352, A390, D396, H464, Q549, Q594, Q410, V526, A174, V208, T427, T456, and P504; F43, Y349, P352, A390, D396, H464, Q549, Q594, Q410, V526, A415, T502, and A139; F43, Y349, P352, A390, D396, H464, Q549, Q594, Q410, V526, A415, T502, 1339, and F446; F43, Y349, P352, A390, D396, H464, Q549, Q594, Q410, V526, T19, D460, Q569, and H596; F43, Y349, P352, A390, D396, H464, Q549, Q594, Q410, V526, D460, S586, E588, and D608; F43, Y349, P352, A390, D396, H464, Q549, Q594, Q410, V526, and D460, relative to SEQ ID NO: 5.

In some embodiments, the second amino acid sequence comprises amino acid substitutions of: F4L, Y23H and A590S, relative to SEQ ID NO: 5. In some embodiments, the second amino acid sequence comprises amino acid substitutions of: T19P, I169L, and 549, relative to SEQ ID NO: 5. In some embodiments, the second amino acid sequence comprises amino acid substitutions of: F43L and A415V, relative to SEQ ID NO: 5. In some embodiments, the second amino acid sequence comprises amino acid substitutions of: G80D and V593M or V593A, relative to SEQ ID NO: 5. In some embodiments, the second amino acid sequence comprises amino acid substitutions of: G80D, 1144V, V593M or V593A, and D606A or D606V, relative to SEQ ID NO: 5. In some embodiments, the second amino acid sequence comprises amino acid substitutions of: M1V, M1I or M1L, T421 or T42A, G80D, V593M or V593A, and D606A or D606V, relative to SEQ ID NO: 5. In some embodiments, the second amino acid sequence comprises amino acid substitutions of: T421 or T42A, G80D, V593M or V593A, and D606A or D606V, relative to SEQ ID NO: 5. In some embodiments, the second amino acid sequence comprises amino acid substitutions of: V156A or V156M, and D604G or D604N, relative to SEQ ID NO: 5. In some embodiments, the second amino acid sequence comprises amino acid substitutions of: A283S or A283T, Y349H, and K365R, relative to SEQ ID NO: 5. In some embodiments, the second amino acid sequence further comprises amino acid substitutions of: D396K and Q594K or Q594L, relative to SEQ ID NO: 5. In some embodiments, the second amino acid sequence further comprises amino acid substitutions of: P352S or P352T, H596Y or H596L, and K131M, relative to SEQ ID NO: 5. In some embodiments, the second amino acid sequence comprises amino acid substitutions of: P352S or P352T and A390V, relative to SEQ ID NO: 5. In some embodiments, the second amino acid sequence comprises amino acid substitutions of: A390V, D396K, and Q594K or Q594L, relative to SEQ ID NO: 5. In some embodiments, the second amino acid sequence comprises amino acid substitutions of: D396K and Q594K or Q594L, relative to SEQ ID NO: 5. In some embodiments, the second amino acid sequence comprises amino acid substitutions of: T456P, T456I, or T456A and T502I, relative to SEQ ID NO: 5. In some embodiments, the second amino acid sequence comprises amino acid substitutions of: H464N or H464R and T502I, relative to SEQ ID NO: 5. In some embodiments, the second amino acid sequence comprises amino acid substitutions of: H464N or H464R and P17T, P17L, or P17S, relative to SEQ ID NO: 5. In some embodiments, the second amino acid sequence comprises amino acid substitutions of: P17T, P17L, or P17S, I235V or I235T, H464N or H464R, and Q569K, Q569L or Q569R, relative to SEQ ID NO: 5. In some embodiments, the second amino acid sequence comprises amino acid substitutions of: I235V or I235T, P352S or P352T, D396K, T456P, T456I, or T456A, and D606A or D606V, relative to SEQ ID NO: 5. In some embodiments, the second amino acid sequence comprises an amino acid sequence having amino acid substitutions of: A415V, T456P, and T502I, relative to SEQ ID NO: 5. In some embodiments, the second amino acid sequence comprises an amino acid sequence having amino acid substitutions of: T456P, T502I, and Q549K, relative to SEQ ID NO: 5. In some embodiments, the second amino acid sequence comprises an amino acid sequence having amino acid substitutions of: I169L, T456P, T502I, and Q549K, relative to SEQ ID NO: 5. In some embodiments, the second amino acid sequence comprises an amino acid sequence having amino acid substitutions of: G80D, T456P, T502I, V593M, and D606A, relative to SEQ ID NO: 5. In some embodiments, the second amino acid sequence comprises an amino acid sequence having amino acid substitutions of: M1V, T42I, G80D, T456P, T502I, V593M, and D606A, relative to SEQ ID NO: 5. In some embodiments, the second amino acid sequence comprises an amino acid sequence having amino acid substitutions of: G80D, 1144V, T456P, T502I, V593M, and D606A, relative to SEQ ID NO: 5. In some embodiments, the second amino acid sequence comprises an amino acid sequence having amino acid substitutions of: T19P, I169L, T456P, T502I, and Q549K, relative to SEQ ID NO: 5. In some embodiments, the second amino acid sequence comprises an amino acid sequence having amino acid substitutions of: F43L, A415V, T456P, and T502I, relative to SEQ ID NO: 5. In some embodiments, the second amino acid sequence comprises an amino acid sequence having amino acid substitutions of: P352T, A390V, D396N, and Q594L, relative to SEQ ID NO: 5. In some embodiments, the second amino acid sequence comprises an amino acid sequence having amino acid substitutions of: P352T, A390V, and D396N, relative to SEQ ID NO: 5. In some embodiments, the second amino acid sequence comprises an amino acid sequence having amino acid substitutions of: A283T, Y349H, D396N, and Q594L, relative to SEQ ID NO: 5. In some embodiments, the second amino acid sequence comprises an amino acid sequence having amino acid substitutions of: A283T, Y349H, P352S, K365R, D396N, Q594L, H596L, and K131M, relative to SEQ ID NO: 5. In select embodiments, the second amino acid sequence comprises an amino acid sequence having one or more amino acid substitutions of: P352T, A390V, D396N, and Q594L, relative to SEQ ID NO: 5.

In select embodiments, the second amino acid sequence comprises an amino acid sequence having one or more amino acid substitutions of: F43S, Y349N or Y349D, P352T, A390V, D396N, H464R, Q549R, Q594L, A415V and T502I, relative to SEQ ID NO: 5. In select embodiments, the second amino acid sequence comprises an amino acid sequence having one or more amino acid substitutions of: F43S, Y349N or Y349D, P352T, A390V, D396N, H464R, Q549R, Q594L, A415V, T502I, and T21A, relative to SEQ ID NO: 5. In select embodiments, the second amino acid sequence comprises an amino acid sequence having one or more amino acid substitutions of: F43S, Y349N or Y349D, P352T, A390V, D396N, H464R, Q549R, Q594L, A415V, T502I, and Q67K, relative to SEQ ID NO: 5. In select embodiments, the second amino acid sequence comprises an amino acid sequence having one or more amino acid substitutions of: F43S, Y349N or Y349D, P352T, A390V, D396N, H464R, Q549R, Q594L, A415V, T502I, T21A, and Q67K, relative to SEQ ID NO: 5.

Any of the polypeptides (e.g., single polypeptides or fusion polypeptides) disclosed herein may further comprise one or more peptides fused to the polypeptide. The one or more peptides encompass both short amino acid sequences or protein or protein domain sequences.

The one or more peptides may comprise a nuclear localization sequence (NLS). The at least one nuclear localization sequence may be appended to the N-terminus, the C-terminus, or embedded in the protein (e.g., inserted internally within the open reading frame (ORF)). The polypeptides may comprise one or more nuclear localization sequences. The nuclear localization sequence may comprise any amino acid sequence known in the art to functionally tag or direct a protein for import into a cell's nucleus (e.g., for nuclear transport). Usually, a nuclear localization sequence comprises one or more positively charged amino acids, such as lysine and arginine.

In some embodiments, the NLS is a monopartite sequence. A monopartite NLS comprises a single cluster of positively charged or basic amino acids. In some embodiments, the monopartite NLS comprises a sequence of K-K/R-X-K/R, wherein X can be any amino acid. Exemplary monopartite NLSs include, without limitation, those from the SV40 large T-antigen (PKKKRKVEDP; SEQ ID NO: 15), c-Myc (PAAKRVKLD; SEQ ID NO: 16), and TUS-proteins (Kaczmarczyk S J et al. 2010). In select embodiments, the NLS comprises a c-Myc NLS.

In some embodiments, the NLS is a bipartite sequence. Bipartite NLSs comprise two clusters of basic amino acids, separated by a spacer of about 9-12 amino acids. Exemplary bipartite NLSs include the NLS of nucleoplasmin, KR[PAATKKAGQA]KKKK (SEQ ID NO: 17), the NLS of EGL-13, MSRRRKANPTKLSENAKKLAKEVEN (SEQ ID NO: 18), the bipartite SV40 NLS, KRTADGSEFESPKKKRKV (SEQ ID NO: 19). In some embodiments, the NLS comprises a bipartite SV40 NLS. In certain embodiments, the NLS comprises an amino acid sequence having at least 70% (e.g., having at least 75%, at least 80%, at least 85%, at least 90%, at least 92%, at least 95%, at least 96%, at least 97%, at least 98%, or at least 99%) similarity to KRTADGSEFESPKKKRKV (SEQ ID NO: 19).

The peptide may comprise an epitope tag (e.g., 3×FLAG tag, an HA tag, a Myc tag, and the like). In some embodiments, the epitope tag may be adjacent, either upstream or downstream, to a nuclear localization sequence. The epitope tags may be at the N-terminus, a C-terminus, or a combination thereof of the corresponding protein.

When the polypeptide is part of a fusion protein, the one or more peptides may be part of or congruent with the linker. In some embodiments, the linker peptide, as described above, further comprises the NLS and/or an epitope tag.

### Methods of Generating and Analyzing Variant CRISPR-Tn Polypeptides

Also provided are methods for generating and analyzing variant CRISPR-Tn polypeptides (e.g., transposon-associated proteins (e.g., TnsA, TnsB, TnsC, TniQ) and Cas proteins (e.g., Cas5, Cas6, Cas7, Cas8). The methods may be directed evolution methods, e.g., by the phage-assisted continuous evolution (PACE) strategies, non-continuous evolution (e.g., PANCE or plate-based strategies), or the methods described herein.

For an overview of PACE technology, see, for example, International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; and U.S. Application, U.S. Ser. No. 13/922,812, filed Jun. 20, 2013; International PCT Application, PCT/US2015/057012, filed Oct. 22, 2015, published as WO 2016/077052 on Sep. 1, 2016; and U.S. Application, U.S. Ser. No. 15/518,639, filed Oct. 22, 2015; International PCT Application, PCT/US2016/043513, filed Jul. 22, 2016, published as WO 2017/015545 on Jan. 26, 2017; and U.S. Application, U.S. Ser. No. 15/216,844, filed Jul. 22, 2016, the entire contents of each of which are incorporated herein by reference.

Variant CRISPR-Tn polypeptides may also be obtained by phage-assisted non-continuous evolution (PANCE), or other plate-based selections. PANCE refers to non-continuous evolution that employs phage as viral vectors. PANCE is a simplified technique for rapid in vivo directed evolution using serial flask transfers of evolving ‘selection phage’ (SP), which contain a gene of interest to be evolved, across fresh E. coli host cells, thereby allowing genes inside the host E. coli to be held constant while genes contained in the SP continuously evolve. Serial flask transfers have long served as a widely-accessible approach for laboratory evolution of microbes, and, more recently, analogous approaches have been developed for bacteriophage evolution. The PANCE system features lower stringency than the PACE system.

Using the evolution strategies and methods provided herein, CRISPR-Tn polypeptides can be evolved to increase modification and integration efficiencies of CRISPR-Tn or CAST systems and methods. In some embodiments, CRISPR-Tn polypeptides can be evolved to target specific nucleic acid sequence of interest.

In some embodiments, the methods comprise exposing nucleic acid sequences encoding two or more different CRISPR-Tn polypeptides to mutagenesis conditions; encoding one or more of TnsA, TnsB, and TnsC polypeptides on a selection phage; encoding crRNA, TniQ, Cas8, Cas7, and Cas6 and any of the TnsA, TnsB, and TnsC polypeptides not included on the selection phage on one or more complementary plasmids; encoding a phage coat protein on an accessory plasmid; and introducing the selection phage, complementary plasmid, and accessory plasmid to a host cell; and screening one or more variant CRISPR-Tn polypeptides expressed by said host.

In some embodiments, TnsA, TnsB, and TnsC polypeptides are on a selection phage and TniQ, Cas8, Cas7, and Cas6 are on one or more complementary plasmids. In some embodiments, TnsA and TnsB polypeptides are on a selection phage and TniQ, Cas8, Cas7, Cas6, and TnsC are on one or more complementary plasmids. In some embodiments, TnsB polypeptide is on a selection phage and TniQ, Cas8, Cas7, Cas6, TnsA, and TnsC are on one or more complementary plasmids.

In some embodiments, the methods select for CRISPR-Tn polypeptides (e.g., TnsA, TnsB, and TnsC, TniQ, Cas8, Cas7, and Cas6) which confer increased targeted integration efficiencies. In some embodiments, the methods select for CRISPR-Tn polypeptides with increased nucleic acid (e.g., target DNA) binding activity. In some embodiments, the methods select for CRISPR-Tn polypeptides with increased binding activity at select target sequences, e.g., select binding at specific protospacer adjacent motifs (PAMs).

In some embodiments, the methods comprise: exposing nucleic acid sequences encoding two or more different CRISPR-Tn polypeptides to mutagenesis conditions; encoding one or more of Cas6, Cas7, Cas8, and TniQ polypeptides on a selection phage; encoding crRNA, TnsA, TnsB, and TnsC and any of the Cas6, Cas7, Cas8, and TniQ polypeptides not included on the selection phage on one or more complementary plasmids; encoding a phage coat protein on an accessory plasmid; and introducing the selection phage, complementary plasmid, and accessory plasmid to a host cell; and screening one or more variant CRISPR-Tn polypeptides expressed by said host. In some embodiments, Cas6, Cas7, Cas8, and TniQ polypeptides are on a selection phage and TnsA, TnsB, and TnsC are on a one or more complementary plasmids.

Selection phage vectors typically comprise a phage genome deficient in a gene required for the generation of infectious phage particles, for example, a phage coat protein, e.g., gIII. In some embodiments, the selection phage comprises a phage genome providing all other phage functions required for the phage life cycle except the gene encoding a phage coat protein.

Thus, the phage coat protein required for the generation of infectious particles is provided on a phage vector separate from the selection phage (e.g., an accessory plasmid or complementary plasmid). In some embodiments, the phage coat protein is encoded on an accessory plasmid. In some embodiment, full length phage coat protein is split between two plasmids. For example, a fragment of the phage coat protein is encoded on an accessory plasmid and the remaining fragment of the phage coat protein is encoded on a complementary plasmid.

Encoding the phage coat protein on two different plasmids minimizes the change of the selection plasmid from acquiring a copy of the phage coat protein due to off-target co-integration as a result of replicative transposition of the components of the CRISPR-Tn system. If the selection plasmid acquired a copy of the phage coat protein, the expression would no longer be contingent on the activity of the proteins encoded by the selection phage.

In some embodiments, crRNA, TniQ, Cas8, Cas7, and Cas6 are encoded on a single complementary plasmid. In some embodiments, crRNA, TniQ, Cas8, Cas7, and Cas6 are encoded on two or more complementary plasmids. In some embodiments, the crRNA is encoded on a complementary plasmid without any additional components. In some embodiments, one or more of TniQ, Cas8, Cas7, and Cas6 are encoded on a single complementary plasmid. In some embodiments, one or more of TniQ, Cas8, Cas7, and Cas6 are encoded on two, three, or four different complementary plasmids. In select embodiments, the crRNA is encoded on a first complementary plasmid and TniQ, Cas8, Cas7, and Cas6 are encoded on a second complementary plasmid.

In some embodiments, the first complementary plasmid further encodes a ribosomal binding site (RBS), a crRNA target, and a T7 RNA polymerase (RNAP) downstream of said crRNA target and RBS. In some embodiments, the first complementary plasmid further encodes an N-terminal phage coat protein (e.g., gIII) fragment linked to a Npu intein (e.g., gIII_{N}-Npu) downstream of a T7 promoter and the accessory plasmid comprises phage coat protein (e.g., gIII) fragment linked to a Npu intein encoded downstream of a crRNA target and RBS.

In some embodiments, the first complementary plasmid further encodes a ribosomal binding site (RBS), a crRNA target. In some embodiments, the first complementary plasmid further encodes an N-terminal phage coat protein (e.g., gIII) fragment linked to a Npu intein (e.g., gIII_{N}-Npu) and the accessory plasmid comprises C-terminal phage coat protein (e.g., gIII) fragment linked to a Npu intein encoded downstream of a crRNA target and RBS.

In some embodiments, crRNA, TnsA, TnsB, and TnsC are encoded on a single complementary plasmid. In some embodiments, crRNA, TnsA, TnsB, and TnsC are encoded on two or more complementary plasmids. In some embodiments, the crRNA is encoded on a complementary plasmid without any additional components. In some embodiments, one or more of TnsA, TnsB, and TnsC are encoded on a single complementary plasmid. In some embodiments, one or more of TnsA, TnsB, and TnsC are encoded on two or three different complementary plasmids. In select embodiments, the crRNA is encoded on a first complementary plasmid and TnsA, TnsB, and TnsC are encoded on a second complementary plasmid.

In some embodiments, the accessory plasmid encodes a C-terminal phage coat protein fragment linked to an intein and the complementary plasmid further encodes a N-terminal phage coat protein fragment linked to an intein downstream of a T7 RNA polymerase (RNAP).

In some embodiments, a complementary plasmid (e.g., a first complementary plasmid or a second complementary plasmid) further comprises a donor cassette. In some embodiments, a plasmid donor comprises a donor cassette. In some embodiments, the crRNA is encoded on a plasmid donor (PD). The donor cassette provides the donor nucleic acid to be integrated downstream of crRNA target.

### Compositions

Compositions comprising the modified transposon-associated proteins and Cas proteins as described herein or a nucleic acid molecule comprising a sequence encoding the modified transposon-associated proteins and Cas proteins are also provided. In some embodiments, the compositions comprise one or more of the disclosed polypeptides, or one or more nucleic acids comprising a sequence encoding one or more of the disclosed polypeptides.

In some embodiments, the compositions comprise a polypeptide comprising an amino acid sequence having at least 70% (e.g., having at least 75%, at least 80%, at least 85%, at least 90%, at least 92%, at least 95%, at least 96%, at least 97%, at least 98%, or at least 99%) identity and one or more amino acid substitutions, deletions, or additions relative to any of SEQ ID NOs: 1-14. In some embodiments, the compositions comprise a polypeptide having one or more or a combination of substitutions as shown in Tables 1-4. In some embodiments, the compositions comprise one or more nucleic acids comprising a sequence encoding a polypeptide comprising an amino acid sequence having at least 70% (e.g., having at least 75%, at least 80%, at least 85%, at least 90%, at least 92%, at least 95%, at least 96%, at least 97%, at least 98%, or at least 99%) identity and one or more amino acid substitutions, deletions, or additions relative to any of SEQ ID NOs: 1-14. In some embodiments, the compositions comprise one or more nucleic acids comprising a sequence encoding a polypeptide having one or more or a combination of substitutions as shown in Tables 1-4.

In some embodiments, the compositions comprise two or more polypeptides comprising an amino acid sequence having at least 70% (e.g., having at least 75%, at least 80%, at least 85%, at least 90%, at least 92%, at least 95%, at least 96%, at least 97%, at least 98%, or at least 99%) identity and one or more amino acid substitutions, deletions, or additions relative to any of SEQ ID NOs: 1-14 (e.g., a first polypeptide having a sequence encoding a TnsA protein of at least 70% (e.g., having at least 75%, at least 80%, at least 85%, at least 90%, at least 92%, at least 95%, at least 96%, at least 97%, at least 98%, or at least 99%) identity to SEQ ID NO: 1 or 4, a second polypeptide having a sequence encoding a TnsB protein of at least 70% (e.g., having at least 75%, at least 80%, at least 85%, at least 90%, at least 92%, at least 95%, at least 96%, at least 97%, at least 98%, or at least 99%) identity to SEQ ID NO: 2 or 5, and/or a third polypeptide having a sequence encoding a TnsC protein of at least 70% (e.g., having at least 75%, at least 80%, at least 85%, at least 90%, at least 92%, at least 95%, at least 96%, at least 97%, at least 98%, or at least 99%) identity to SEQ ID NO: 3 or 6, or alternatively a first polypeptide having a sequence encoding a Cas8 protein of at least 70% (e.g., having at least 75%, at least 80%, at least 85%, at least 90%, at least 92%, at least 95%, at least 96%, at least 97%, at least 98%, or at least 99%) identity to SEQ ID NO: 8 or 12, a second polypeptide having a sequence encoding a Cas7 protein of at least 70% (e.g., having at least 75%, at least 80%, at least 85%, at least 90%, at least 92%, at least 95%, at least 96%, at least 97%, at least 98%, or at least 99%) identity to SEQ ID NO: 9 or 13, and/or a third polypeptide having a sequence encoding a Cas6 protein of at least 70% (e.g., having at least 75%, at least 80%, at least 85%, at least 90%, at least 92%, at least 95%, at least 96%, at least 97%, at least 98%, or at least 99%) identity to SEQ ID NO: 10 or 14).

In some embodiments, the compositions comprise one, two, or more polypeptides having one or more of the amino acid substitutions, deletions, or additions relative to SEQ ID NOs: 1-14 as shown in Tables 1-4. In some embodiments, the compositions comprise one or more nucleic acids comprising a sequence encoding two or more polypeptides comprising an amino acid sequences having at least 70% (e.g., having at least 75%, at least 80%, at least 85%, at least 90%, at least 92%, at least 95%, at least 96%, at least 97%, at least 98%, or at least 99%) identity and one or more amino acid substitutions, deletions, or additions relative to any of SEQ ID NOs: 1-14. In some embodiments, the compositions comprise one or more nucleic acids comprising a sequence encoding two or more polypeptides having one or more or a combination of substitutions as shown in Tables 1-4.

In some embodiments, the composition comprises two or all of: a first polypeptide having an amino acid sequence encoding a TnsA protein of least 70% (e.g., having at least 75%, at least 80%, at least 85%, at least 90%, at least 92%, at least 95%, at least 96%, at least 97%, at least 98%, or at least 99%) identity to SEQ ID NO: 1 with one or more amino acid substitutions, deletions, or additions relative to SEQ ID NO: 1; a second polypeptide having an amino acid sequence encoding a TnsB protein of at least 70% (e.g., having at least 75%, at least 80%, at least 85%, at least 90%, at least 92%, at least 95%, at least 96%, at least 97%, at least 98%, or at least 99%) identity to SEQ ID NO: 2 with one or more amino acid substitutions, deletions, or additions relative to SEQ ID NO: 2; or a third polypeptide having an amino acid sequence encoding a TnsC protein of an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 3 with one or more amino acid substitutions, deletions, or additions relative to SEQ ID NO: 3, or one or more nucleic acids comprising a sequence encoding thereof.

In some embodiments, the first polypeptide comprises one or more amino acid substitutions at positions: 2, 3, 5, 28, 57, 77, 80, 107, 110, 116, 122, 142, 155, 161, 166, 173, 177, 185, 211, 216, 227, and 230, relative to SEQ ID NO: 1. In some embodiments, the first polypeptide comprises an amino acid sequence having one or more amino acid substitutions of: A2T, T3I, L5S, T28A, A57T, F77L, Y80D, K107M, K107R, Y110C, Y110D, D116G, E122A, D142E, M155I, K161R, N166D, K173E, Y177N, Y177D, C185R, D211Y, K216E, A227P, G230D and G230S, relative to SEQ ID NO: 1.

In some embodiments, the first polypeptide comprises an amino acid sequence having amino acid substitutions at positions: 2 and 230; 107 and 166; 107, 166, and one or both of: 2 and 227; 211 and 110 or 142; 110, 155 and 230; 122 and 155; 155 and 177, relative to SEQ ID NO: 1. In some embodiments, the first polypeptide comprises an amino acid sequence having amino acid substitutions of: A2T, and G230D or G230S, relative to SEQ ID NO: 1. In some embodiments, the first polypeptide comprises an amino acid sequence having amino acid substitutions of: K107M and N166D, relative to SEQ ID NO: 1. In some embodiments, the first polypeptide further comprises amino acid substitutions of: A2T and/or Y177N or Y177D, relative to SEQ ID NO: 1. In some embodiments, the first polypeptide comprises an amino acid sequence having amino acid substitutions of: D211Y and Y110C, Y110D, or D142E, relative to SEQ ID NO: 1. In some embodiments, the first polypeptide comprises an amino acid sequence having amino acid substitutions of: Y110C or Y110D, M155I, and G230D or G230S, relative to SEQ ID NO: 1. In some embodiments, the first polypeptide comprises an amino acid sequence having amino acid substitutions of: E122A and M155I, relative to SEQ ID NO: 1. In some embodiments, the first polypeptide comprises an amino acid sequence having amino acid substitutions of: M155I and Y177N or Y177D, relative to SEQ ID NO: 1.

In some embodiments, the second polypeptide comprises one or more amino acid substitutions at positions: 2, 5, 22, 24, 25, 29, 75, 141, 199, 215, 319, 347, 364, 370, 383, 439, 454, 458, 485, 509, 533, 538, 565, 581, 586, 595, 596, 597, and 600, relative to SEQ ID NO: 2. In some embodiments, the second polypeptide comprises an amino acid sequence having one or more amino acid substitutions of: A2T, A2S, G5R, S22P, E24D, L25I, A29S, P75T, I141T, V199I, S215R, D319V, Y347F, S364N, E370K, N383D, V439A, E454D, E454G, S458N, V485F, R509G, D533A, A538V, H565Y, A581T, H586L, N595K, D596N, D597N, D597Y, and I600V, relative to SEQ ID NO: 2. In some embodiments, the second polypeptide comprises an amino acid sequence having amino acid substitutions at positions: 2 and 597; 24 and 25; 24, 25, 458, 509, 565, and 600; 75 and 597; 141, 454, 533 and 595; 581, 370, and 454; 370 and 581; 370 and 454; 458 and 509; 458, 509 and 565; 458, 509, 565, and 600; 565, 586, and 596; or 565, 509, 458, 600 and at least one of 24, 25, 29, 215, 319, 364, 383, and 586, relative to SEQ ID NO: 2.

In some embodiments, the second polypeptide comprises an amino acid sequence having amino acid substitutions of: A2T or A2S, and D597N or D597Y, relative to SEQ ID NO: 2. In some embodiments, the second polypeptide comprises an amino acid sequence having amino acid substitutions of: E24D and L25I, relative to SEQ ID NO: 2. In some embodiments, the second polypeptide comprises an amino acid sequence having amino acid substitutions of: E24D and L25I, S458N, R509G, H565Y, and I600V, relative to SEQ ID NO: 2. In some embodiments, the second polypeptide comprises an amino acid sequence having amino acid substitutions of: P75T and D597N or D597Y, relative to SEQ ID NO: 2. In some embodiments, the second polypeptide comprises an amino acid sequence having amino acid substitutions of: E454D or E454G, D533A, N595K, relative to SEQ ID NO: 2. In some embodiments, the second polypeptide comprises an amino acid sequence having amino acid substitutions of: E370K, E454D or E454G, and A581T, relative to SEQ ID NO: 2. In some embodiments, the second polypeptide comprises an amino acid sequence having amino acid substitutions of: E370K, and A581T, E454D, or E454G, relative to SEQ ID NO: 2. In some embodiments, the second polypeptide comprises an amino acid sequence having amino acid substitutions of: S458N and R509G, relative to SEQ ID NO: 2. In some embodiments, the second polypeptide further comprises amino acid substitutions of: H565Y and/or I600V. In some embodiments, the second polypeptide comprises an amino acid sequence having amino acid substitutions of: H565Y, H586L, and D596N, relative to SEQ ID NO: 2. In some embodiments, the second polypeptide comprises amino acid substitutions of: H565Y, R509G, S458N, I600V and at least one of E24D, L25I, A29S, S215R, D319V, S364N, N383D, and H586L, relative to SEQ ID NO: 2.

In some embodiments, the third polypeptide comprises one or more amino acid substitutions at positions: 9, 15, 16, 18, 21, 64, 81, 86, 87, 99, 109, 142, 147, 153, 168, 180, 216, 230, 285, and 304, relative to SEQ ID NO: 3. In some embodiments, the third polypeptide comprises an amino acid sequence having one or more amino acid substitutions of: 19V, A15V, F16Y, S18F, S21N, N64D, H81Y, D86Y, N87K, V99I, E109D, E142K, V147I, N153D, I168M, A180E, A216S, L230F, K285E, and R304R, relative to SEQ ID NO: 3. In some embodiments, the third polypeptide comprises an amino acid sequence having amino acid substitutions at positions: 142 and 216, relative to SEQ ID NO: 3. In some embodiments, the third polypeptide comprises an amino acid sequence having amino acid substitutions of: E142K and A216S, relative to SEQ ID NO: 3.

In some embodiments, the first polypeptide comprises an amino acid sequence having amino acid substitutions of: A2T and G230D, relative to SEQ ID NO: 1 and the second polypeptide comprises an amino acid sequence having amino acid substitutions of: A2T and D597N, relative to SEQ ID NO: 2.

In some embodiments, the first polypeptide comprises an amino acid sequence having amino acid substitutions of: M155I and Y177N, relative to SEQ ID NO: 1 and the second polypeptide comprises an amino acid sequence having amino acid substitutions of: E370K and A581T, relative to SEQ ID NO: 2.

In some embodiments, the first polypeptide comprises an amino acid sequence having amino acid substitutions of: M155I, relative to SEQ ID NO: 1 and the second polypeptide comprises an amino acid sequence having amino acid substitutions of: A2S and D596N, relative to SEQ ID NO: 2.

In some embodiments, the first polypeptide comprises an amino acid sequence having amino acid substitutions of: M155I, relative to SEQ ID NO: 1 and the second polypeptide comprises an amino acid sequence having amino acid substitutions of: S22P, Y347F, and E454G, relative to SEQ ID NO: 2.

In some embodiments, the first polypeptide comprises an amino acid sequence having amino acid substitutions of: D211Y, and D142E or Y110C, relative to SEQ ID NO: 1 and the third polypeptide comprises an amino acid sequence having amino acid substitutions of: F16Y, relative to SEQ ID NO: 3.

In some embodiments, the first polypeptide comprises an amino acid sequence having amino acid substitutions of: E122A and M155I, relative to SEQ ID NO: 1, the second polypeptide comprises an amino acid sequence having amino acid substitutions of: V485F, relative to SEQ ID NO: 2, and third polypeptide comprises an amino acid sequence having amino acid substitutions of: A15V, S21N and D86Y, relative to SEQ ID NO: 3.

In some embodiments, the composition comprises two or all of: a first polypeptide having an amino acid sequence encoding a TnsA protein of at least 70% (e.g., having at least 75%, at least 80%, at least 85%, at least 90%, at least 92%, at least 95%, at least 96%, at least 97%, at least 98%, or at least 99%) identity to SEQ ID NO: 4 with one or more amino acid substitutions, deletions, or additions relative to SEQ ID NO: 4; a second polypeptide having an amino acid sequence encoding a TnsB protein of at least 70% (e.g., having at least 75%, at least 80%, at least 85%, at least 90%, at least 92%, at least 95%, at least 96%, at least 97%, at least 98%, or at least 99%) identity to SEQ ID NO: 5 with one or more amino acid substitutions, deletions, or additions relative to SEQ ID NO: 5; and a third polypeptide having an amino acid sequence encoding a TnsC protein of at least 70% (e.g., having at least 75%, at least 80%, at least 85%, at least 90%, at least 92%, at least 95%, at least 96%, at least 97%, at least 98%, or at least 99%) identity to SEQ ID NO: 6 with one or more amino acid substitutions, deletions, or additions relative to SEQ ID NO: 6, or one or more nucleic acids comprising a sequence encoding thereof.

In some embodiments, the composition comprises: a first polypeptide having an amino acid sequence encoding a TnsA protein of at least 70% (e.g., having at least 75%, at least 80%, at least 85%, at least 90%, at least 92%, at least 95%, at least 96%, at least 97%, at least 98%, or at least 99%) identity to SEQ ID NO: 4 with one or more amino acid substitutions, deletions, or additions relative to SEQ ID NO: 4; a second polypeptide having an amino acid sequence encoding a TnsB protein of at least 70% (e.g., having at least 75%, at least 80%, at least 85%, at least 90%, at least 92%, at least 95%, at least 96%, at least 97%, at least 98%, or at least 99%) identity to SEQ ID NO: 5 with one or more amino acid substitutions, deletions, or additions relative to SEQ ID NO: 5; and a third polypeptide having an amino acid sequence encoding a TnsC protein of SEQ ID NO: 6.

In some embodiments, the composition comprises two or all of: a first polypeptide having an amino acid sequence encoding a TnsA protein of SEQ ID NO: 4; a second polypeptide having an amino acid sequence encoding a TnsB protein of at least 70% (e.g., having at least 75%, at least 80%, at least 85%, at least 90%, at least 92%, at least 95%, at least 96%, at least 97%, at least 98%, or at least 99%) identity to SEQ ID NO: 5 with one or more amino acid substitutions, deletions, or additions relative to SEQ ID NO: 5; and a third polypeptide having an amino acid sequence encoding a TnsC protein of at least 70% (e.g., having at least 75%, at least 80%, at least 85%, at least 90%, at least 92%, at least 95%, at least 96%, at least 97%, at least 98%, or at least 99%) identity to SEQ ID NO: 6 with one or more amino acid substitutions, deletions, or additions relative to SEQ ID NO: 6, or one or more nucleic acids comprising a sequence encoding thereof.

In some embodiments, the first polypeptide comprises one or more amino acid substitutions at positions: 4, 5, 9, 10, 12, 21, 23, 25, 26, 31, 32, 34, 35, 37, 41, 45, 47, 48, 51, 52, 55, 60, 61, 65, 67, 69, 72, 75, 79, 80, 82, 87, 88, 90, 91, 93, 94, 96, 98, 99, 100, 103, 106, 108, 113, 116, 125, 126, 128, 129, 135, 139, 143, 146, 147, 149, 153, 154, 156, 158, 159, 160, 162, 164, 166, 167, 168, 169, 170, 177, 179, 180, 182, 183, 185, 187, 188, 190, 191, 192, 193, 195, 196, 200, 204, 207, and 208, relative to SEQ ID NO: 4. In some embodiments, the first polypeptide comprises an amino acid sequence having one or more amino acid substitutions of: R4K, N5K, P9S, A10P, N12D, T21I, V23M, S25N, S25R, V26M, V26G, S31N, S32I, E34A, F35L, A37D, H41L, D45N, I47V, E48G, G51V, S52I, E55K, E55D, E60K, F61L, S65T, S65A, P67T, P67L, P67S, P67H, T69A, A72V, A72D, S75I, S75R, S75T, K79E, T80P, K82E, K87R, P88L, P88T, P88A, S90F, K91N, K91E, A93T, A93S, S94N, L96P, R98Q, A99D, A99V, E100K, A103T, A106T, S108A, I113F, V116F, V116I, V125M, V125A, N126T, 1128V, 1128L, L129P, L135M, S139N, S139G, G143V, G143C, G146D, G146S, I147V, K149E, K149T, K149R, S153I, S153R, S153N, F154C, H156R, H156L, S158N, S158R, G159V, V160A, K162R, N164D, I166L, S167I, S168I, S168R, S168N, Q169R, V170M, V170G, V170L, T177I, T177A, S179R, F180C, F180L, F182C, F182L, G183S, M185I, K187R, G188D, V190I, K191N, A192S, D193N, G195V, G195D, G195S, C196W, T200A, T204I, A207V, A207T, and T208I, relative to SEQ ID NO: 4.

In some embodiments, the first polypeptide comprises an amino acid sequence having amino acid substitutions at positions: 108 and 47 or 208; 170 and 207; 88 and 147; 47, 88 and 147; 88, 147, 170, and 182; 88, 147, 170, 182, and 51 or 180; 88, 147, and 154; 88, 128, 147, 170, and 182; or 170, 207, and 108, relative to SEQ ID NO: 4. In some embodiments, the first polypeptide comprises an amino acid sequence having amino acid substitutions of: S108A, and I47V or T208I, relative to SEQ ID NO: 4. In some embodiments, the first polypeptide comprises an amino acid sequence having amino acid substitutions of: V170M, and A207V or A207T, relative to SEQ ID NO: 4. In some embodiments, the first polypeptide further comprises amino acid substitutions of: S108A, V170M, and A207V or A207T, relative to SEQ ID NO: 4. In some embodiments, the first polypeptide comprises an amino acid sequence having amino acid substitutions of: P88T, I147V, V170L, and F182L, relative to SEQ ID NO: 4. In some embodiments, the first polypeptide comprises an amino acid sequence having amino acid substitutions of: P88T, I147V, V170L, F182L and G51V or F180L, relative to SEQ ID NO: 4. In some embodiments, the first polypeptide comprises an amino acid sequence having amino acid substitutions of: P88T, I147V, and F154C, relative to SEQ ID NO: 4.

In some embodiments, the second polypeptide comprises one or more amino acid substitutions at positions: 1, 2, 4, 5, 8, 9, 10, 11, 12, 13, 14, 15, 16, 17, 19, 20, 21, 22, 23, 24, 25, 26, 27, 28, 29, 30, 32, 33, 36, 37, 39, 40, 41, 42, 43, 44, 45, 49, 52, 55, 56, 58, 60, 62, 63, 67, 71, 74, 76, 78, 79, 80, 81, 82, 83, 84, 85, 86, 87, 88, 89, 91, 92, 95, 97, 100, 101, 104, 106, 110, 112, 113, 115, 117, 119, 120, 124, 125, 127, 129, 130, 131, 134, 139, 142, 144, 145, 146, 147, 149, 150, 155, 156, 157, 158, 159, 163, 164, 165, 167, 169, 173, 174, 176, 181, 182, 186, 187, 190, 195, 197, 198, 205, 208, 209, 211, 215, 218, 223, 226, 227, 231, 232, 235, 239, 246, 248, 250, 259, 260, 261, 262, 263, 267, 269, 273, 274, 277, 278, 280, 281, 282, 283, 285, 287, 288, 290, 295, 298, 302, 303, 307, 313, 316, 317, 320, 323, 325, 331, 332, 339, 345, 348, 349, 352, 353, 354, 356, 361, 362, 363, 364, 365, 366, 367, 369, 370, 371, 372, 373, 375, 376, 380, 383, 385, 386, 389, 390, 392, 396, 397, 399, 402, 403, 404, 407, 408, 410, 411, 412, 413, 414, 415, 416, 421, 422, 423, 424, 425, 426, 427, 428, 429, 430, 431, 434, 435, 437, 440, 443, 445, 446, 448, 450, 452, 456, 459, 460, 463, 464, 470, 472, 473, 494, 495, 498, 501, 502, 504, 505, 506, 508, 509, 510, 512, 513, 514, 517, 520, 521, 522, 525, 526, 527, 530, 531, 532, 533, 535, 537, 538, 540, 541, 542, 543, 544, 545, 546, 547, 548, 549, 550, 551, 552, 553, 554, 556, 557, 558, 559, 560, 561, 562, 563, 564, 565, 567, 568, 569, 570, 571, 574, 575, 576, 580, 582, 583, 584, 585, 586, 587, 588, 589, 590, 591, 592, 593, 594, 595, 596, 597, 599, 600, 601, 602, 603, 604, 606, 607, 608, 611, 613, 618, 620, and 656, relative to SEQ ID NO: 5.

In some embodiments, the second polypeptide comprises an amino acid sequence having one or more amino acid substitutions of: M1V, M1I, M1L, T2I, T2A, F4L, F5L, F8L, F8V, F8S, D9N, E10K, E10D, S11I, S11R, S11G, L12P, V13M, V13G, V13E, V13L, P14L, L15Q, K16N, K16R, P17T, P17L, P17S, T19I, T19S, T19A, T19P, P20S, P20L, T21A, Q22R, Y23H, V24M, K25R, L26M, D27A, D27G, D28N, D28Y, A29T, A29V, N30K, I32F, I32S, Q33H, L36M, D37A, D37Y, F39L, S40P, D41E, T42I, T42K, T42A, F43L, F43S, F43V, K44N, N45D, N45S, Q49R, K52Q, S55A, T56A, D58E, K60Q, S62T, R63K, R63G, Q67R, Q67H, Q67K, D71Y, K74R, E76K, F78C, K79R, G80V, G80D, G81S, G81V, G81D, D82N, V83G, V83M, V83A, V84A, V84G, R85G, R85K, P86L, N87S, R89C, V91G, V91A, A92V, A92T, R95K, K97R, E100D, S101A, D104V, A106D, A106T, D110N, N112H, H113Y, M115R, N117Y, T119A, N120D, N120K, N120S, G124V, D125N, D125E, K127R, F129L, D130N, K131M, E134D, E134G, A139S, A139T, P142S, 1144V, A145S, A145T, T146A, A147V, Q149R, Y150H, I155L, V156A, V156L, V156M, K157V, E158A, N159S, V163G, E164A, E164G, E164D, G165D, I167V, I169L, I169T, N173S, N173H, N173T, A174S, A174T, N176D, A181S, I182L, I182V, I182T, A186E, A186T, V187G, V187A, A190T, A190S, F195S, A197P, D198G, D198N, A205S, V208M, P209T, T211I, E215D, E218D, P223S, P223H, L226V, I227V, D231N, E232K, I235V, I235T, R239G, I246V, V248E, V248M, S250I, S259N, Y260C, K261R, S262N, P263L, S267N, A269V, T273I, T273N, H274Y, K277N, K277R, P278S, S280T, L281M, D282E, D282N, A283T, A283S, N285S, E287D, L288M, N290K, F295S, F298I, F298S, V302I, V303M, A307S, N313S, H316R, A317V, S320N, S320R, I323L, I325V, R331K, K332E, I339V, V345L, V345M, E348K, Y349H, Y349D, Y349N, Y349C, P352S, P352T, E353Q, E353D, L354M, G356S, N361D, I362V, I362T, L363P, L363T, L363M, E364G, K365R, E366G, E367G, K369N, K369E, K369M, P370S, E371K, V372M, D373G, I375V, M376I, T380P, T380A, E383K, E383D, F385L, H386Y, I389V, A390V, A390I, V392I, D396N, D396G, D396K, S397P, S399N, S399G, T402I, R403G, R403I, R403K, R403S, I404T, I404V, K407R, K407E, R408K, Q410K, Q410H, Q410R, Q411H, G412V, F413L, D414N, A415V, A415T, Y416C, M421I, N422K, E423K, E423D, E424A, E425K, E426D, T427A, T427S, R428K, F429L, S430A, M431L, R434H, R434C, R434S, I435V, D437G, D437N, T440S, T440I, R443C, G445S, F446L, F446I, Y448C, E450D, E450G, M452I, T456P, T456A, T456I, A459T, D460N, K463N, H464N, H464R, H464S, E470K, V472M, V472A, K473D, K473N, E494D, E494G, S495A, E498A, E498K, C501Y, T502I, T502S, P504S, P504L, T505A, G506Y, G506D, G506L, G506S, T508A, D509E, D509Y, C510Y, S512N, I513L, I513V, I513F, Y514H, K517M, K517N, K517Q, K520R, K521N, I522T, I522V, I522F, E525K, V526E, V526M, I527V, S530N, S530R, K531T, D532G, D532Y, S533Y, G535D, A537T, K538R, K538N, R540K, R540G, M541L, A542T, I543L, H544R, E545A, R546G, R546K, V547M, K548Q, K548R, Q549K, Q549R, E550A, Q551K, E552D, E552K, V553I, F554V, E556K, E556G, S557A, K558R, T559P, T559I, T559A, K560R, A561T, A561G, K562R, K562N, I563L, T564I, A565S, A565V, K567R, K568N, K568R, Q569K, Q569L, Q569R, A570V, Q571R, D574N, V575M, V575A, S576R, T580I, T580A, T582I, T582S, I583V, K584R, V585M, S586P, S586A, S586F, E587A, E588K, E588G, E588D, S589I, S589R, S589N, A590S, A590T, A591V, P592L, V593M, V593A, Q594L, K595R, K595N, H596Y, H596L, H596P, I597T, I597V, N599H, D600L, D600N, D600G, D600V, N601S, N601K, S602A, S602P, S602Y, D603A, D603V, D604G, D604Y, D604N, D606A, D606V, D606Y, D607Y, D607E, D608N, A611T, E613D, R618I, T620P, and A656V, relative to SEQ ID NO: 5.

In some embodiments, the second polypeptide comprises an amino acid sequence having amino acid substitutions at positions: 4, 23 and 590; 19, 169, and 549; 43 and 415; 80 and 593; 80, 144, 593, and 606; 1, 42, 80, 593, and 606; 42, 80, 593, and 606; 156 and 604; 283, 349, and 365; 283, 349, 365, 396, and 594; 283, 349, 365, 396, 594, 596, and 131; 352 and 390; 390, 396, and 594; 396 and 594; 456 and 502; 464 and 502; 464 and 17; 17, 235, 464, and 596; 235, 352, 396, 456, and 606; 415, 456, and 502; 456, 502, and 549; 169, 456, 502, and 549; 80, 456, 502, 593, and 606; 1, 42, 80, 456, 502, 593, and 606; 80, 144, 456, 502, 593, and 606; 19, 169, 456, 502 and 549; 43, 415, 456, and 502; 352, 390, 396, and 594; 352, 390, and 396; 283, 349, 396, and 594; 11, 55, 120, 362, 584, 600, and 604; 43, 84, 144, 349, and 517; 164 and 165; 164 and 173; 362 and 446; 352, 390, 396, 549, and 594; 352, 390, 396, 464, 549, and 594; 43, 349, 352, 390, 396, 464, 549, and 594; 43, 349, 352, 390, 396, 464, 549, 594 and one or more positions selected from 63, 145, 174, 182, 208, 410, 427, 456, 504, and 526; 43, 352, 390, 396, 464, 549, and 594; 43, 349, 352, 390, 396, 464, 549, 594, 410, 526, 415, and 502; 43, 349, 352, 390, 396, 464, 549, 594, 410, 526, 415, 502, and 21; 43, 349, 352, 390, 396, 464, 549, 594, 410, 526, 415, 502, and 67; 43, 349, 352, 390, 396, 464, 549, 594, 410, 526, 415, 502, 21, and 67; 43, 349, 352, 390, 396, 464, 549, 594, 410, 526, 174, 208, 427, 456, and 504; 43, 349, 352, 390, 396, 464, 549, 594, 410, 526, 415, 502, and 139; 43, 349, 352, 390, 396, 464, 549, 594, 410, 526, 415, 502, 339, and 446; 43, 349, 352, 390, 396, 464, 549, 594, 410, 526, 19, 460, 569, and 596; 43, 349, 352, 390, 396, 464, 549, 594, 410, 526, 460, 586, 588, and 608; 43, 349, 352, 390, 396, 464, 549, 594, 410, 526, and 460; 352, 390, 396, 549, 586, and 594; 63, 158, 352, 390, 396, 549, 586, and 594; 164, 165, 352, 363, 390, 396, 410, 549, 586, and 594; 164, 173, 352, 390, 396, 549, 586, and 594; 83, 352, 390, 396, 549, 586, and 594; 8, 43, 174, 349, 352, 390, 396, 427, 464, 549, and 594; or 283, 349, 365, 396, 594, 596, and 131, relative to SEQ ID NO: 5.

In select embodiments, the second polypeptide comprises amino acid substitutions at positions: 352, 390, 396, 594, or any combination thereof, relative to SEQ ID NO: 5.

In select embodiments, the second polypeptide comprises amino acid substitutions at positions: F43, Y349, P352, A390, D396, H464, Q549, Q594, and T456; F43, Y349, P352, A390, D396, H464, Q549, Q594, T456, and V526; F43, Y349, P352, A390, D396, H464, Q549, Q594, and P504; F43, Y349, P352, A390, D396, H464, Q549, Q594, and V526; F43, Y349, P352, A390, D396, H464, Q549, Q594, Q410, and V526; F43, Y349, P352, A390, D396, H464, Q549, Q594, A174, and T427; F43, Y349, P352, A390, D396, H464, Q549, Q594, and V208; or F43, Y349, P352, A390, D396, H464, Q549, Q594, R63, A145, 1182, and V526; F43, Y349, P352, A390, D396, H464, Q549, Q594, Q410, V526, A415, and T502; F43, Y349, P352, A390, D396, H464, Q549, Q594, Q410, V526, A415, T502, and T21; F43, Y349, P352, A390, D396, H464, Q549, Q594, Q410, V526, A415, T502, and Q67; F43, Y349, P352, A390, D396, H464, Q549, Q594, Q410, V526, A415, T502, T21, and Q67; F43, Y349, P352, A390, D396, H464, Q549, Q594, Q410, V526, A174, V208, T427, T456, and P504; F43, Y349, P352, A390, D396, H464, Q549, Q594, Q410, V526, A174, V208, T427, T456, and P504; F43, Y349, P352, A390, D396, H464, Q549, Q594, Q410, V526, A415, T502, and A139; F43, Y349, P352, A390, D396, H464, Q549, Q594, Q410, V526, A415, T502, 1339, and F446; F43, Y349, P352, A390, D396, H464, Q549, Q594, Q410, V526, T19, D460, Q569, and H596; F43, Y349, P352, A390, D396, H464, Q549, Q594, Q410, V526, D460, S586, E588, and D608; F43, Y349, P352, A390, D396, H464, Q549, Q594, Q410, V526, and D460, relative to SEQ ID NO: 5.

In some embodiments, the second polypeptide comprises an amino acid sequence having amino acid substitutions of: F4L, Y23H and A590S, relative to SEQ ID NO: 5. In some embodiments, the second polypeptide comprises an amino acid sequence having amino acid substitutions of: T19P, I169L, and 549, relative to SEQ ID NO: 5. In some embodiments, the second polypeptide comprises an amino acid sequence having amino acid substitutions of: F43L and A415V, relative to SEQ ID NO: 5. In some embodiments, the second polypeptide comprises an amino acid sequence having amino acid substitutions of: G80D and V593M or V593A, relative to SEQ ID NO: 5. In some embodiments, the second polypeptide comprises an amino acid sequence having amino acid substitutions of: G80D, 1144V, V593M or V593A, and D606A or D606V, relative to SEQ ID NO: 5. In some embodiments, the second polypeptide comprises an amino acid sequence having amino acid substitutions of: M1V, M1I or M1L, T421 or T42A, G80D, V593M or V593A, and D606A or D606V, relative to SEQ ID NO: 5. In some embodiments, the second polypeptide comprises an amino acid sequence having amino acid substitutions of: T421 or T42A, G80D, V593M or V593A, and D606A or D606V, relative to SEQ ID NO: 5. In some embodiments, the second polypeptide comprises an amino acid sequence having amino acid substitutions of: V156A or V156M, and D604G or D604N, relative to SEQ ID NO: 5. In some embodiments, the second polypeptide comprises an amino acid sequence having amino acid substitutions of: A283S or A283T, Y349H, and K365R, relative to SEQ ID NO: 5. In some embodiments, the second polypeptide further comprises amino acid substitutions of: D396K and Q594K or Q594L, relative to SEQ ID NO: 5. In some embodiments, the second polypeptide further comprises amino acid substitutions of: P352S or P352T, H596Y or H596L, and K131M, relative to SEQ ID NO: 5. In some embodiments, the second polypeptide comprises an amino acid sequence having amino acid substitutions of: P352S or P352T and A390V, relative to SEQ ID NO: 5. In some embodiments, the second polypeptide comprises an amino acid sequence having amino acid substitutions of: A390V, D396K, and Q594K or Q594L, relative to SEQ ID NO: 5. In some embodiments, the second polypeptide comprises an amino acid sequence having amino acid substitutions of: D396K and Q594K or Q594L, relative to SEQ ID NO: 5. In some embodiments, the second polypeptide comprises an amino acid sequence having amino acid substitutions of: T456P, T456I, or T456A and T502I, relative to SEQ ID NO: 5. In some embodiments, the second polypeptide comprises an amino acid sequence having amino acid substitutions of: H464N or H464R and T502I, relative to SEQ ID NO: 5. In some embodiments, the second polypeptide comprises an amino acid sequence having amino acid substitutions of: H464N or H464R and P17T, P17L, or P17S, relative to SEQ ID NO: 5. In some embodiments, the second polypeptide comprises an amino acid sequence having amino acid substitutions of: P17T, P17L, or P17S, I235V or I235T, H464N or H464R, and Q569K, Q569L or Q569R, relative to SEQ ID NO: 5. In some embodiments, the second polypeptide comprises an amino acid sequence having amino acid substitutions of: I235V or I235T, P352S or P352T, D396K, T456P, T456I, or T456A, and D606A or D606V, relative to SEQ ID NO: 5. In some embodiments, the second polypeptide comprises an amino acid sequence having amino acid substitutions of: A415V, T456P, and T502I, relative to SEQ ID NO: 5. In some embodiments, the second polypeptide comprises an amino acid sequence having amino acid substitutions of T456P, T502I, and Q549K, relative to SEQ ID NO: 5. In some embodiments, the second polypeptide comprises an amino acid sequence having amino acid substitutions of: I169L, T456P, T502I, and Q549K, relative to SEQ ID NO: 5. In some embodiments, the second polypeptide comprises an amino acid sequence having amino acid substitutions of: G80D, T456P, T502I, V593M, and D606A, relative to SEQ ID NO: 5. In some embodiments, the second polypeptide comprises an amino acid sequence having amino acid substitutions of: M1V, T421, G80D, T456P, T502I, V593M, and D606A, relative to SEQ ID NO: 5. In some embodiments, the second polypeptide comprises an amino acid sequence having amino acid substitutions of: G80D, 1144V, T456P, T502I, V593M, and D606A, relative to SEQ ID NO: 5. In some embodiments, the second polypeptide comprises an amino acid sequence having amino acid substitutions of: T19P, I169L, T456P, T502I, and Q549K, relative to SEQ ID NO: 5. In some embodiments, the second polypeptide comprises an amino acid sequence having amino acid substitutions of: F43L, A415V, T456P, and T502I, relative to SEQ ID NO: 5. In some embodiments, the second polypeptide comprises an amino acid sequence having amino acid substitutions of: P352T, A390V, D396N, and Q594L, relative to SEQ ID NO: 5. In some embodiments, the second polypeptide comprises an amino acid sequence having amino acid substitutions of: P352T, A390V, and D396N, relative to SEQ ID NO: 5. In some embodiments, the second polypeptide comprises an amino acid sequence having amino acid substitutions of: A283T, Y349H, D396N, and Q594L, relative to SEQ ID NO: 5. In some embodiments, the second polypeptide comprises an amino acid sequence having amino acid substitutions of: A283T, Y349H, P352S, K365R, D396N, Q594L, H596L, and K131M, relative to SEQ ID NO: 5. In select embodiments, the second polypeptide comprises an amino acid sequence having one or more amino acid substitutions of: P352T, A390V, D396N, and Q594L, relative to SEQ ID NO: 5.

In some embodiments, the second polypeptide comprises an amino acid sequence having amino acid substitutions of: F43S, Y349N, P352T, A390V, D396N, H464R, Q549R, Q594L, and T456I, relative to SEQ ID NO: 5. In some embodiments, the second polypeptide comprises an amino acid sequence having amino acid substitutions of: F43S, Y349N, P352T, A390V, D396N, H464R, Q549R, Q594L, T456P, and V526E, relative to SEQ ID NO: 5. In some embodiments, the second polypeptide comprises an amino acid sequence having amino acid substitutions of: F43S, Y349D, P352T, A390V, D396N, H464R, Q549R, Q594L, and P504S, relative to SEQ ID NO: 5. In some embodiments, the second polypeptide comprises an amino acid sequence having amino acid substitutions of: F43S, Y349N, P352T, A390V, D396N, H464R, Q549R, Q594L, and V526E, relative to SEQ ID NO: 5. In some embodiments, the second polypeptide comprises an amino acid sequence having amino acid substitutions of: F43S, Y349N, P352T, A390V, D396N, H464R, Q549R, Q594L, Q410K, and V526E, relative to SEQ ID NO: 5. In some embodiments, the second polypeptide comprises an amino acid sequence having amino acid substitutions of: F43S, Y349N, P352T, A390V, D396N, H464R, Q549R, Q594L, A174S, and T427S, relative to SEQ ID NO: 5. In some embodiments, the second polypeptide comprises an amino acid sequence having amino acid substitutions of: F43S, Y349N, P352T, A390V, D396N, H464R, Q549R, Q594L, and V208M, relative to SEQ ID NO: 5. In some embodiments, the second polypeptide comprises an amino acid sequence having amino acid substitutions of: F43S, Y349N, P352T, A390V, D396N, H464R, Q549R, Q594L, R63G, A145S, I182T, and V526E, relative to SEQ ID NO: 5.

In some embodiments, the second polypeptide comprises an amino acid sequence having amino acid substitutions of: F43S, Y349N or Y349D, P352T, A390V, D396N, H464R, Q549R, Q594L, A415V and T502I, relative to SEQ ID NO: 5. In some embodiments, the second polypeptide comprises an amino acid sequence having amino acid substitutions of: F43S, Y349N or Y349D, P352T, A390V, D396N, H464R, Q549R, Q594L, A415V, T502I, and T21A, relative to SEQ ID NO: 5. In some embodiments, the second polypeptide comprises an amino acid sequence having amino acid substitutions of: F43S, Y349N or Y349D, P352T, A390V, D396N, H464R, Q549R, Q594L, A415V, T502I, and Q67K, relative to SEQ ID NO: 5. In some embodiments, the second polypeptide comprises an amino acid sequence having amino acid substitutions of: F43S, Y349N or Y349D, P352T, A390V, D396N, H464R, Q549R, Q594L, A415V, T502I, T21A, and Q67K, relative to SEQ ID NO: 5.

In some embodiments, the third polypeptide comprises one or more amino acid substitutions at positions: 1, 2, 3, 5, 6, 7, 9, 11, 12, 14, 21, 22, 26, 27, 31, 35, 38, 43, 44, 46, 47, 54, 59, 60, 61, 64, 65, 67, 68, 71, 72, 74, 76, 79, 80, 81, 84, 89, 95, 102, 105, 109, 110, 111, 112, 113, 114, 116, 118, 119, 120, 123, 129, 130, 131, 132, 134, 142, 145, 146, 147, 148, 150, 154, 155, 166, 169, 178, 180, 181, 183, 184, 187, 190, 194, 197, 201, 204, 207, 209, 213, 219, 221, 225, 226, 227, 229, 232, 233, 234, 236, 238, 241, 246, 251, 252, 256, 257, 261, 263, 265, 267, 269, 271, 272, 274, 280, 281, 285, 286, 288, 291, 292, 296, 299, 301, 303, 304, 306, 307, 308, 310, 313, 314, 316, 317, 318, 319, 320, 323, 324, 326, 328, 330, 331, 332, 340, 341, 343, 344, 355, 412, 418, 427, 514, 1198, 1201, 1206, 1212, 1260, and 1282, relative to SEQ ID NO: 6. In some embodiments, the third polypeptide does not include substitutions at one or more positions selected from: 6, 9, 28, 44, 64, 76, 80, 95, 110, 113, 114, 116, 118, 130, 132, 142, 155, 187, 190, 194, 221, 233, 234, 238, 261, 272, 280, 281, 299, 303, 304, 307, 308, 313, 316, and 328, relative to SEQ ID NO: 6.

In some embodiments, the third polypeptide comprises an amino acid sequence having one or more amino acid substitutions of: M1L, M1V, N2S, A3T, T5P, T5A, T5S, E6D, 17S, 17V, 19F, Q11R, L12M, N14D, N14S, M21I, H22P, H22Y, K26N, K26R, T27I, M31I, L35R, N38S, S43P, D44N, D44G, Q46L, C47S, T54I, S59T, H60Y, T61A, H64Y, Y65H, K67N, K67R, R68Q, A71G, T72A, N74D, S76C, S76Y, T79I, M80I, P81S, V84L, R89L, A95D, A95T, A102T, E105D, E105K, S109N, S109R, S110P, Q111R, 1112T, K113N, K113E, K114N, K114M, K114E, G116D, K118N, K118R, T119I, D120V, K123N, L129M, I130V, K131R, A132S, K134M, K134N, F142V, L145M, I146T, E147K, F148S, S150F, R154K, Q155H, E166D, K169E, P178S, A180V, A181T, A181S, I183V, A184S, A184T, A184V, P187S, A190T, A190V, V194M, V194A, R197I, Y201N, L204M, D207N, K209N, Q213H, Q213V, A219S, K221N, D225N, V226E, P227T, K229E, S232N, K233N, K233R, N234H, T236A, A238V, A238S, A241S, E246D, K251N, H252Y, H252R, E256D, A257S, A261V, S263I, S263N, N265D, Y267C, E269K, E269D, K271E, K271R, H272Y, I274V, F280L, D281N, D281G, K285G, K286N, K288R, S291F, S291P, K292N, K296R, K296N, I299S, D301G, E303D, I304T, I304V, E306G, V307L, V307G, V307A, V307D, V307G, I308N, N310S, Y313H, N314K, N316K, N316D, A317D, L318Q, D319N, P320S, P320L, M323I, L324M, D326N, V328M, V328A, A330D, I331V, V332G, S340L, T341A, A343G, S344N, I355V, F412V, V418F, Y427C, R514K, S1198L, A1201V, G1206S, C1212G, F1260L, and V1282M, relative to SEQ ID NO: 6. In some embodiments, the third polypeptide does not include one or more substitutions selected from: E6D, 19F, I28T, D44N, D44G, H64Y, S76Y, M80I, A95T, S110P, K113N, K113E, K114N, K114E, G116D, K118N, K118R, I130V, A132S, F142V, Q155H, P187S, A190T, V194A, K221N, K233R, N234H, A238V, A261V, H272Y, F280L, D281G, I299D, E303F, I304T, V307S, I308N, Y313H, N316D, and V328M, relative to SEQ ID NO: 6.

In some embodiments, the third polypeptide comprises an amino acid sequence having amino acid substitutions at positions: 2, 67, 95, and 226; 6 and 316; 38, 95, 303; 44 and 118; 67, 95, and 226; 44 and 76; 44, 76, and 118; 130, 234, 303; 118 and 1201; 118, 1201, and 44; 118, 1201, and 76; 130, 234, and 303; 154 and 269; 221 and 44; 44, 76, 130, 234, and 303; 44, 76, 118, and 1201; 197 and 314; 76, 181, and 194; 76, 118, 252, and 292; 76 and 274; 76, 102, 118, and 307; 12 and 76; 67, 95, and 226; 26 and 76; 22, 76, 319; 154 and 269; 76 and 238; 76, 238, 296, and 328; 7 and 76; 76 and 263; 59, 76, 306, and 316; or 280 and 340; relative to SEQ ID NO: 6. In select embodiments, the polypeptide comprises an amino acid sequence having an amino acid substitution at position 76, relative to SEQ ID NO: 6.

In some embodiments, the third polypeptide comprises an amino acid sequence having amino acid substitutions of: N2S, K67N or K67R, A95D, and V226E, relative to SEQ ID NO: 6. In some embodiments, the third polypeptide comprises an amino acid sequence having amino acid substitutions of: E6D and N316K or N316D, relative to SEQ ID NO: 6. In some embodiments, the third polypeptide comprises an amino acid sequence having amino acid substitutions of: N38S, A95D, E303D, relative to SEQ ID NO: 6. In some embodiments, the third polypeptide comprises an amino acid sequence having amino acid substitutions of: K67N or K67R, A95D, and V226E, relative to SEQ ID NO: 6. In some embodiments, the third polypeptide comprises an amino acid sequence having amino acid substitutions of: D44G or D44N and S76Y or K118N or K118R, relative to SEQ ID NO: 6. In some embodiments, the third polypeptide comprises an amino acid sequence having amino acid substitutions of: D44G or D44N, I130V, N234H, E303D, relative to SEQ ID NO: 6. In some embodiments, the third polypeptide comprises an amino acid sequence having amino acid substitutions of: K118N or K118R and A1201V, relative to SEQ ID NO: 6. In some embodiments, the third polypeptide further comprises amino acid substitutions of: D44G or D44N or S76Y. In some embodiments, the third polypeptide comprises an amino acid sequence having amino acid substitutions of: I130V, N234H, and E303D, relative to SEQ ID NO: 6. In some embodiments, the third polypeptide comprises an amino acid sequence having amino acid substitutions of: R154K and E269K or E269D, relative to SEQ ID NO: 6. In some embodiments, the third polypeptide comprises an amino acid sequence having amino acid substitutions of: K221N and D44G or D44N, relative to SEQ ID NO: 6. In some embodiments, the third polypeptide comprises an amino acid sequence having amino acid substitutions of: F280L and S340L, relative to SEQ ID NO: 6. In some embodiments, the third polypeptide comprises an amino acid sequence having amino acid substitutions of: D44N or D44G, S76Y, 1130V, N234H, and E303D, relative to SEQ ID NO: 6. In some embodiments, the third polypeptide comprises an amino acid sequence having amino acid substitutions of: D44N or D44G, S76Y, K118R, and A1201V, relative to SEQ ID NO: 6. In select embodiments, the third polypeptide comprises an amino acid sequence having an amino acid substitutions of: S76Y, relative to SEQ ID NO: 6. In select embodiments, the third polypeptide comprises an amino acid sequence having an amino acid substitutions of: R197I and N314K, relative to SEQ ID NO: 6. In select embodiments, the third polypeptide comprises an amino acid sequence having an amino acid substitutions of: S76Y, A181S, and V194M, relative to SEQ ID NO: 6. In select embodiments, the third polypeptide comprises an amino acid sequence having an amino acid substitutions of: S76Y, K118R, H252R, and K292N, relative to SEQ ID NO: 6. In select embodiments, the third polypeptide comprises an amino acid sequence having an amino acid substitutions of: S76Y and I274V, relative to SEQ ID NO: 6. In select embodiments, the third polypeptide comprises an amino acid sequence having an amino acid substitutions of: S76Y, A102T, K118R, and V307G, relative to SEQ ID NO: 6. In select embodiments, the third polypeptide comprises an amino acid sequence having an amino acid substitutions of: L12M and S76Y, relative to SEQ ID NO: 6. In select embodiments, the third polypeptide comprises an amino acid sequence having an amino acid substitutions of: K67N, A95D, and V226E, relative to SEQ ID NO: 6. In select embodiments, the third polypeptide comprises an amino acid sequence having an amino acid substitutions of: K26N and S76Y, relative to SEQ ID NO: 6. In select embodiments, the third polypeptide comprises an amino acid sequence having an amino acid substitutions of: H22Y, S76Y, and D319N, relative to SEQ ID NO: 6. In select embodiments, the third polypeptide comprises an amino acid sequence having an amino acid substitutions of: R154K and E269D, relative to SEQ ID NO: 6. In select embodiments, the third polypeptide comprises an amino acid sequence having an amino acid substitutions of: S76Y and A238S, relative to SEQ ID NO: 6. In select embodiments, the third polypeptide comprises an amino acid sequence having an amino acid substitutions of: S76Y and S263N, relative to SEQ ID NO: 6. In select embodiments, the third polypeptide comprises an amino acid sequence having an amino acid substitutions of: S59T, S76Y, E306G, and N316D, relative to SEQ ID NO: 6. In select embodiments, the third polypeptide comprises an amino acid sequence having an amino acid substitutions of: S76Y, A238S, K296N, and V328M, relative to SEQ ID NO: 6. In select embodiments, the third polypeptide comprises an amino acid sequence having an amino acid substitutions of: S76Y and S263N, relative to SEQ ID NO: 6. In select embodiments, the third polypeptide comprises an amino acid sequence having an amino acid substitutions of: L12M and S76Y, relative to SEQ ID NO: 6. In select embodiments, the third polypeptide comprises an amino acid sequence having an amino acid substitutions of: I7V and S76Y, relative to SEQ ID NO: 6.

In some embodiments, the first polypeptide comprises an amino acid sequence having amino acid substitutions of: P88T and I147V, relative to SEQ ID NO: 4, the second polypeptide comprises an amino acid sequence having amino acid substitutions of: P352T, A390V, D396N, Q549R, and Q594L, relative to SEQ ID NO: 5, and the third polypeptide comprises an amino acid sequence having amino acid substitutions of: S76Y and K296N, relative to SEQ ID NO: 6.

In some embodiments, the second polypeptide comprises an amino acid sequence having amino acid substitutions of: G80V, P352T, A390V, D396N, Q594L, and H596L, relative to SEQ ID NO: 5, and the third polypeptide comprises an amino acid sequence having amino acid substitutions of: L12M and S76Y, relative to SEQ ID NO: 6.

In some embodiments, the first polypeptide comprises an amino acid sequence having amino acid substitutions of: S25R and T177A, relative to SEQ ID NO: 4, the second polypeptide comprises an amino acid sequence having amino acid substitutions of: P352T, K365R, A390V, D396N, S530G, D574R, and Q594L relative to SEQ ID NO: 5, and the third polypeptide comprises an amino acid sequence having amino acid substitutions of: S76Y and A317D, relative to SEQ ID NO: 6.

Any or all of the first polypeptide, the second polypeptide, and/or the third polypeptide may be linked in a fusion protein. In specific embodiments, the first and second polypeptide are linked in a fusion protein.

In some embodiments, the composition comprises two or more of: a first polypeptide having an amino acid sequence encoding a TniQ protein of having at least 70% (e.g., having at least 75%, at least 80%, at least 85%, at least 90%, at least 92%, at least 95%, at least 96%, at least 97%, at least 98%, or at least 99%) identity to SEQ ID NO: 7 with one or more amino acid substitutions, deletions, or additions relative to SEQ ID NO: 7; a second polypeptide having an amino acid sequence encoding a Cas8 protein of at least 70% (e.g., having at least 75%, at least 80%, at least 85%, at least 90%, at least 92%, at least 95%, at least 96%, at least 97%, at least 98%, or at least 99%) identity to SEQ ID NO: 8 with one or more amino acid substitutions, deletions, or additions relative to SEQ ID NO: 8; a third polypeptide having an amino acid sequence encoding a Cas7 protein of at least 70% (e.g., having at least 75%, at least 80%, at least 85%, at least 90%, at least 92%, at least 95%, at least 96%, at least 97%, at least 98%, or at least 99%) identity to SEQ ID NO: 9 with one or more amino acid substitutions, deletions, or additions relative to SEQ ID NO: 9; and a fourth polypeptide having an amino acid sequence encoding a Cas6 protein of at least 70% (e.g., having at least 75%, at least 80%, at least 85%, at least 90%, at least 92%, at least 95%, at least 96%, at least 97%, at least 98%, or at least 99%) identity to SEQ ID NO: 10 with one or more amino acid substitutions, deletions, or additions relative to SEQ ID NO: 10, or one or more nucleic acids comprising a sequence encoding thereof.

In some embodiments, the first polypeptide comprises one or more amino acid substitutions at positions: 99, 133, 189, 265, 266, 336, and 343, relative to SEQ ID NO: 7. In some embodiments, the first polypeptide comprises an amino acid sequence having one or more amino acid substitutions of: M99I, S189N, H265Q, A266V, L336F, and V343A, relative to SEQ ID NO: 7.

In some embodiments, the second polypeptide comprises one or more amino acid substitutions at positions: 119, 134, 155, 180, 183, 274, 319, 447, 454, 458, 461, 512, 538, and 580, relative to SEQ ID NO: 8. In some embodiments, the second polypeptide comprises an amino acid sequence having one or more amino acid substitutions of: Y119H, N134R, N134Q, D155N, Q180R, D183N, R274L, N319D, V447I, A454S, E458G, D461N, A512T, D538K, and P580Q, relative to SEQ ID NO: 8.

In some embodiments, the third polypeptide comprises one or more amino acid substitutions at positions: 28, 82, 144, 151, 162, 182, 273, 327, 346, relative to SEQ ID NO: 9. In some embodiments, the third polypeptide comprises an amino acid sequence having one or more amino acid substitutions of: R28K, A82T, K144, C151R, N162S, K182E, D273G, A327D, and M346I, relative to SEQ ID NO: 9.

In some embodiments, the fourth polypeptide comprises one or more amino acid substitutions at positions: 21 and 90, relative to SEQ ID NO: 10. In some embodiments, the fourth polypeptide comprises an amino acid sequence having one or more amino acid substitutions of: A21S and V90A, relative to SEQ ID NO: 10.

In some embodiments, the composition comprises two or more of: a first polypeptide having an amino acid sequence encoding a TniQ protein of least 70% (e.g., having at least 75%, at least 80%, at least 85%, at least 90%, at least 92%, at least 95%, at least 96%, at least 97%, at least 98%, or at least 99%) identity to SEQ ID NO: 11 with one or more amino acid substitutions, deletions, or additions relative to SEQ ID NO: 11; a second polypeptide having an amino acid sequence encoding a Cas8 protein of at least 70% (e.g., having at least 75%, at least 80%, at least 85%, at least 90%, at least 92%, at least 95%, at least 96%, at least 97%, at least 98%, or at least 99%) identity to SEQ ID NO: 12 with one or more amino acid substitutions, deletions, or additions relative to SEQ ID NO: 12; a third polypeptide having an amino acid sequence encoding a Cas7 protein of least 70% (e.g., having at least 75%, at least 80%, at least 85%, at least 90%, at least 92%, at least 95%, at least 96%, at least 97%, at least 98%, or at least 99%) identity to SEQ ID NO: 13 with one or more amino acid substitutions, deletions, or additions relative to SEQ ID NO: 13; and a fourth polypeptide having an amino acid sequence encoding a Cas6 protein of least 70% (e.g., having at least 75%, at least 80%, at least 85%, at least 90%, at least 92%, at least 95%, at least 96%, at least 97%, at least 98%, or at least 99%) identity to SEQ ID NO: 14 with one or more amino acid substitutions, deletions, or additions relative to SEQ ID NO: 14, one or more nucleic acids comprising a sequence encoding thereof.

In some embodiments, the first polypeptide comprises one or more amino acid substitutions at positions: 2, 3, 7, 9, 11, 12, 14, 16, 20, 26, 29, 32, 34, 35, 40, 43, 45, 46, 54, 61, 64, 65, 70, 77, 101, 103, 105, 106, 108, 109, 111, 119, 120, 123, 126, 127, 130, 131, 148, 149, 151, 157, 159, 164, 166, 185, 194, 196, 203, 211, 217, 218, 219, 236, 242, 257, 267, 279, 283, 286, 288, 291, 293, 296, 303, 306, 313, 314, 316, 326, 331, 336, 347, 352, 361, 374, 377, 395, 396, 398, and 408, relative to SEQ ID NO: 11. In some embodiments, the first polypeptide comprises an amino acid sequence having one or more amino acid substitutions of: A2T, F3S, P7R, A9S, A9G, A11G, F12I, D14N, S16Y, Y20H, S26N, F29S, S32N, E34K, G35V, G35S, G35D, I40S, E43D, H45P, E46K, A54S, R61W, V64M, Y65C, N70S, A77T, D101N, K103E, N105K, N105D, S106G, V108M, A109G, Y111N, L119M, R120S, R123S, A126T, E127G, V130M, D131N, Q148R, S149Y, H151Y, A157D, T159I, A164V, L166M, T185A, S194G, A196T, T203A, K211R, E217K, R218K, R218S, N219S, A236T, E242D, N257K, N267S, M279I, M279V, D283G, N286S, T288I, K291Q, I293V, D296N, S303I, S303G, K306N, S310Y, S310P, I313T, Y314F, A316T, E326G, T331I, A336V, A347T, A347S, T352S, Y361H, M374T, M374I, R377G, T395I, S396T, S396F, G398V, and A408V, relative to SEQ ID NO: 11. In some embodiments, the first polypeptide comprises an amino acid sequence having one or more amino acid substitutions at positions: 105, 109, 131, 148, 279, and 310; or 9, 105, 109, 131, 148, 279, and 310, relative to SEQ ID NO: 11. In some embodiments, the first polypeptide comprises an amino acid sequence having one or more amino acid substitutions of: N105K, A109G, D131N, Q148R, M279I, and S310Y or S310P, relative to SEQ ID NO: 11. In some embodiments, the first polypeptide comprises an amino acid sequence having one or more amino acid substitutions of: A9S, N105K, A109G, D131N, A148R, M279I, and S310P, relative to SEQ ID NO: 11.

In some embodiments, the second polypeptide comprises one or more amino acid substitutions at positions: 4, 5, 6, 8, 9, 11, 12, 13, 16, 17, 20, 21, 24, 26, 28, 29, 34, 37, 38, 41, 49, 54, 59, 60, 63, 65, 67, 74, 77, 81, 88, 92, 93, 94, 96, 102, 105, 106, 108, 110, 121, 126, 128, 134, 138, 142, 147, 150, 151, 153, 156, 157, 160, 162, 165, 170, 171, 173, 174, 179, 181, 183, 185, 186, 187, 188, 191, 198, 201, 206, 207, 226, 228, 233, 236, 241, 249, 250, 256, 267, 268, 270, 275, 276, 277, 279, 283, 286, 289, 303, 305, 306, 310, 312, 314, 315, 316, 323, 326, 329, 349, 353, 355, 356, 357, 358, 361, 370, 372, 373, 376, 378, 382, 388, 391, 397, 399, 403, 405, 419, 421, 423, 424, 425, 427, 428, 430, 431, 432, 433, 449, 457, 473, 477, 480, 485, 487, 489, 494, 496, 497, 498, 500, 502, 509, 511, 515, 518, 519, 520, 540, 545, 550, 555, 557, 570, 571, 580, 583, 585, 590, 594, 603, 607, 608, 611, 617, 620, 624, 636, 639, 641, 642, 644, 646, 655, 658, 660, 663, 665, 668, 672, 673, 678, 682, 685, 688, and 695, relative to SEQ ID NO: 12. In some embodiments, the second polypeptide comprises an amino acid sequence having one or more amino acid substitutions of: K4N, E5K, L6M, L6I, E8K, E8D, 19T, D11N, T12A, T13I, D16G, R17C, R17S, R20K, R20E, R21E, R21K, S24K, S24Q, S24R, Y26S, Y26H, A28S, A28D, M29I, G34D, A37S, V38M, V38G, I41V, R49L, D54G, K59R, K60N, K63N, A65T, A65V, K67E, K74E, K77E, W81C, K88R, K88E, 192T, R93E, R93K, V94M, K96N, E102D, E102G, T105A, L106M, S108P, V110A, G121S, S126P, K128R, L134M, Y138S, Q142H, W147L, K150N, V151M, V151L, A153T, S156R, S156G, D157N, K160R, K160E, A162T, S165N, S165G, V170E, K171E, F173V, K174N, K174R, T179A, K181T, S183N, P185T, E186K, E186D, E187K, A188S, A188V, D191Y, D191E, R198H, R198C, R198S, R201K, D206G, G207D, A226T, I228V, R233K, N236T, R241E, A249S, A250S, I256T, S267G, S267N, K268N, H270P, S275N, S275G, R276G, A277D, A277S, A277T, K279N, G283D, V286G, V289M, G303D, I305T, F306S, A310D, A310T, A312G, A312D, A312T, K314N, Q315R, R316G, N323S, E326A, E326K, N329S, G349D, E353D, L355M, L355R, E356G, E356D, S357P, A358V, R361S, P370T, N372K, E373D, S376F, T378I, F382L, M388V, G391S, R397K, A399S, K403N, M405I, L419P, D421N, K423R, H424N, H424R, V425L, I427V, E428K, D430A, D431G, E432D, H433N, A449T, G457D, R473K, E477D, G480D, F485L, S487R, S487G, S489N, N494D, S496N, A497S, V498G, K500N, K502N, Q509R, A511T, A511E, R515S, R518S, P519T, G520D, G520V, Y540C, Q545H, K550N, K555E, H557Q, P570S, E571D, C580R, S583R, E585K, E585G, E590D, R594K, M6031, H607N, H607L, K608R, D611N, L617P, N620S, K624N, T636P, M639V, N641S, V642G, S644N, S644G, E646D, A655V, V658M, K660N, T663A, T665I, R668S, I672V, G673V, S678R, M682L, A685V, A685D, K688N, and V695M, relative to SEQ ID NO: 12.

In some embodiments, the second polypeptide comprises an amino acid sequence having amino acid substitutions at positions: 134, 179, 185, 540, 555, 624, and 646; 138, 250, 275, and 421; 303, 405, 520, and 590; 134, 179, 185, 540, 555, and 646, 4 and 49; 4 and 388; 4 and 571; 4, 162, and 480; 4 and 315; 5 and 316; 17 and 156; 38, 108, 497 and 583; 59, 157 and 644; 96, 305, 550 and 642; 106, 160 and 228; 312, 424, 449 and 457; or 376 and 611, relative to SEQ ID NO: 12. In some embodiments, the second polypeptide comprises an amino acid sequence having amino acid substitutions of: L134M, T179A, P185T, Y540C, K555E, K624N, and E646D, relative to SEQ ID NO: 12. In some embodiments, the second polypeptide comprises an amino acid sequence having amino acid substitutions of: Y138S, A250S, S275N, and D421N, relative to SEQ ID NO: 12. In some embodiments, the second polypeptide comprises an amino acid sequence having amino acid substitutions of: L134M, T179A, P185T, Y540C, K555E, and E646D, relative to SEQ ID NO: 12. In some embodiments, the second polypeptide comprises an amino acid sequence having amino acid substitutions of: G303D, M405I, G520D, and E590D, relative to SEQ ID NO: 12.

In some embodiments, the third polypeptide comprises one or more amino acid substitutions at positions: 5, 10, 11, 26, 30, 35, 40, 42, 45, 46, 47, 58, 61, 65, 71, 72, 75, 77, 78, 80, 82, 83, 94, 98, 113, 115, 116, 117, 121, 128, 133, 138, 146, 148, 161, 171, 175, 177, 182, 184, 191, 193, 201, 203, 211, 212, 219, 225, 226, 232, 233, 235, 236, 237, 238, 240, 250, 274, 282, 286, 292, 295, 304, 307, 309, 312, 313, 315, 316, 317, 318, 320, 321, 322, 323, 328, 340, 343, 344, 345, 347, 348, 349, and 350, relative to SEQ ID NO: 13. In some embodiments, the third polypeptide comprises an amino acid sequence having one or more amino acid substitutions of: N5K, N5T, D10N, R11K, D26N, V30E, D35N, R40L, P42A, G45S, G45V, F46V, T47R, T47S, N58T, P61L, T65I, T71I, T71R, T71D, L72M, C75S, V77A, P78L, N80T, E82D, H83Y, H83N, A94S, V98M, E113D, C121F, A128S, A155S, E116D, T117I, R133K, G138V, N146D, G148V, C161R, A171V, A171S, K175T, A177V, K182E, L184M, 1191V, S193A, S193F, F201S, S203N, E211K, A212V, Y219R, N225S, N225T, D226Y, E232K, E232Q, A233N, A233S, A233K, K235R, Q236R, Q236S, F237L, V238Q, V238M, A240T, A240V, S250A, R274G, A282V, I286N, I286T, I286F, P292S, S295N, K304R, E307D, Y309C, A312V, L313M, N315K, N315T, N315S, C316G, I317V, T318A, T318P, K320R, N321D, E322K, K323N, I328T, M340I, K343E, K343R, K344E, K344R, A345T, A345D, A345S, A345Y, A345R, A345K, A345E, A345G, A347K, A347S, A347D, K348N, K349R, A350K, A350D, A350V, and A350T, relative to SEQ ID NO: 13.

In some embodiments, the third polypeptide comprises an amino acid sequence having amino acid substitutions at positions: 30, 46, 240, 304, and 316; 30, 46, 240, and 316; 42 and 318; 184, 240, 315, and 345; 211 and 274; 237 and 237; 286 and 350; 317 and 347; 171, 286, and 315; or 328 and 350, relative to SEQ ID NO: 13. In some embodiments, the third polypeptide comprises an amino acid sequence having amino acid substitutions of: V30E, F46V, A240T or A240V, K304R, and C316G, relative to SEQ ID NO: 13. In some embodiments, the third polypeptide comprises an amino acid sequence having amino acid substitutions of: V30E, F46V, A240T or A240V, and C316G, relative to SEQ ID NO: 13. In some embodiments, the third polypeptide comprises an amino acid sequence having amino acid substitutions of: L184M, A240T or A240V, N315K, and A345T, relative to SEQ ID NO: 13. In some embodiments, the third polypeptide comprises an amino acid sequence having amino acid substitutions of: 1286N and A350D, relative to SEQ ID NO: 13. In some embodiments, the third polypeptide comprises an amino acid sequence having amino acid substitutions of: A171S, I286F, and N315S, relative to SEQ ID NO: 13.

In some embodiments, the third polypeptide comprises an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 13 and at least one amino acid substitution with a positively charged amino acid. In some embodiments, the positively charged amino acid is arginine. In some embodiments, the at least one amino acid substitution is at position 2, 5, 6, 7, 8, 9, 10, 12, 38, 39, 40, 41, 42, 43, 44, 45, 46, 47, 48, 64, 65, 66, 67, 68, 69, 70, 222, 224, 225, 227, 228, 229, 231, 232, 233, 234, 235, 255, 256, 257, 258, 277, 286, 287, 337, 338, 339, 340, 345, 346, 347, 348, 349, 350, or a combination thereof, relative to SEQ ID NO: 13. In some embodiments, the at least one amino acid substitution is at positions: 346 and 348; 346, 348 and 349; 346, 348, 349, an 350; 350 and 351; 350, 351, and 352; 350, 351, 352, and 353; 235 and 227; 235 and 345; 235 and 346; 235 and 347; 235 and 348; 235 and 349; 235 and 350; 235, 227, and 349; 5, 235 and 346; 5, 235 and 348; 5, 235 and 349; 227, 235, and 346; or 227, 235, and 348, relative to SEQ ID NO: 13.

In some embodiments, the fourth polypeptide comprises one or more amino acid substitutions at positions: 2, 9, 13, 14, 15, 34, 38, 42, 46, 50, 59, 60, 73, 75, 77, 82, 83, 85, 86, 97, 110, 115, 120, 124, 130, 132, 134, 140, 143, 145, 156, 159, 162, 164, 177, 199, 232, and 270, relative to SEQ ID NO: 14. In some embodiments, the fourth polypeptide comprises an amino acid sequence having one or more amino acid substitutions of: Q2K, H9L, K13E, Q14K, A15G, K34N, E38K, V42I, A46D, S50I, V59G, Y60H, A73S, A73T, F75L, D77G, G82S, F83L, F83V, F83C, K85E, V86I, E97, I110S, I110L, S115R, K120N, K124R, G130D, D132E, N134T, A140T, E143K, D145G, S156I, E159K, I162V, H164Y, H164F, Y177C, S199I, S232L, and L270S, relative to SEQ ID NO: 14.

In some embodiments, fourth polypeptide comprises an amino acid sequence having one or more amino acid substitutions at positions: 82, 110, 115, 164, and 199, relative to SEQ ID NO: 14. In some embodiments, fourth polypeptide comprises an amino acid sequence having one or more amino acid substitutions of: G82S, I110S, S115R, H164Y or H164F, and S199I, relative to SEQ ID NO: 14. In some embodiments, fourth polypeptide comprises an amino acid sequence having one or more amino acid substitutions at positions: 110, 115, 164, 199, and 124, relative to SEQ ID NO: 14. In some embodiments, fourth polypeptide comprises an amino acid sequence having one or more amino acid substitutions of: I110S, S115R, K124R, H164Y or H164F, and S199I, relative to SEQ ID NO: 14. In some embodiments, the fourth polypeptide comprises an amino acid sequence having one or more amino acid substitutions at positions: 82, 110, 115, 124, 164, and 199, relative to SEQ ID NO: 14. In some embodiments, the fourth polypeptide comprises an amino acid sequence having one or more amino acid substitutions of: G82S, I110S, S115R, K124R, H164Y, and S199I, relative to SEQ ID NO: 14. In some embodiments, the fourth polypeptide comprises an amino acid sequence having one or more amino acid substitutions at positions: 110, 115, and 164, relative to SEQ ID NO: 14. In some embodiments, the fourth polypeptide comprises an amino acid sequence having one or more amino acid substitutions of: I110S, S115R, and H164F, relative to SEQ ID NO: 14. In some embodiments, the fourth polypeptide comprises an amino acid sequence having one or more amino acid substitutions at positions: 110, 115, 164, and 199, relative to SEQ ID NO: 14. In some embodiments, the fourth polypeptide comprises an amino acid sequence having one or more amino acid substitutions of: I110S, S115R, H164F, and S199I, relative to SEQ ID NO: 14. In some embodiments, the fourth polypeptide comprises an amino acid sequence having one or more amino acid substitutions at positions: 110, 115, 164, 199, and 124, relative to SEQ ID NO: 14. In some embodiments, the fourth polypeptide comprises an amino acid sequence having one or more amino acid substitutions of: I110S, S115R, H164F, S199I, and K124R, relative to SEQ ID NO: 14.

Any or all of the first polypeptide, the second polypeptide, the third polypeptide, and/or the fourth polypeptide may be linked in a fusion protein.

In some embodiments, the compositions further comprise one or more Cas proteins. Examples of Cas proteins include, but are not limited to: Cas1, Cas1B, Cas2, Cas3, Cas4, Cas5, Cas6, Cas7, Cas8, Cas9 (also known as Csn1 and Csx12), Cas10, Cas 11, Cas12a (formerly Cpf1), Cas12b (formerly C2c1), Cas12c (formerly C2c3), Cas12d (formerly CasY), Cas12e (formerly CasX), Cas12k (formerly C2c5), Cas13a (formerly known as C2c2), Cas13b, Cas13c, Cas13d, homologs, orthologs, paralogs, modified versions, either engineered or naturally occurring, or active fragments thereof. The Cas proteins may be selected from the group consisting of Cas5, Cas6, Cas7, Cas8, Cas9, and Cas12, or variants thereof.

Any Cas protein known in the art can be employed in the compositions described herein, as appropriate. Cas proteins are described in detail in: U.S. Pat. Nos. 8,697,359, 8,771,945, 8,945,839, 9,688,971, and 11,441,137; International Patent Publications: WO2016106239, WO2016205749, WO2017106657, WO2017070605, WO2017127807, WO2017184768, WO2017219027, WO2018170333, WO2019089796, WO2019089804, WO2019089820, WO2019104058, WO2020033601, WO2020181264, WO2020191102, WO2020257715, WO2021146641, WO2021216512, and WO2022159822; and Makarova et al., Nature Reviews Microbiology, 9 (6): 467-477 (2011); Wiedenheft et al., Nature, 482:331-338 (2012); Gasiunas et al., Proceedings of the National Academy of Sciences USA, 109 (39): E2579-E2586 (2012); Jinek et al., Science, 337:816-821 (2012); Carroll, Molecular Therapy, 20 (9): 1658-1660 (2012); Al-Attar et al., Biol Chem., 392 (4): 277-289 (2011); Hale et al., Molecular Cell, 45 (3): 292-302 (2012), and Zhang Y., Pathog Dis. 2017; 75 (4): ftx036. doi: 10.1093/femspd/ftx036.

In some embodiments, the at least one Cas protein is derived from a Type I CRISPR-Cas system (e.g., Type I-F, Type I-B). Type I CRISPR-Cas systems encode a multi-subunit protein-RNA complex called Cascade, which utilizes a crRNA (or guide RNA) to target double-stranded DNA during an immune response. In some embodiments, the at least one Cas protein comprises Cas5, Cas6, Cas7, and Cas8.

In some embodiments, the at least one Cas protein is derived from a Type II CRISPR-Cas system. Type II CRISPR-Cas systems are considered to be the minimal CRISPR-Cas system that includes the CRISPR repeat-spacer array and only four, but often three, cas genes with cas9 being responsible for encoding the large multidomain protein Cas9 that is sufficient for targeting and cleaving DNA. In some embodiments, the at least one Cas protein comprises Cas9.

In some embodiments, the at least one Cas protein is derived from a Type V CRISPR-Cas system. Type V CRISPR-Cas systems are distinguished by a single RNA-guided RuvC domain-containing effector, Cas12. In some embodiments, the at least one Cas protein comprises Cas12.

In some embodiments, the Cas protein is catalytically inactive. For example, in some embodiments, the Cas protein is a Cas nickase, such as Cas9 nickase (Cas9n). A Cas nickase protein is typically engineered through inactivating point mutation(s) in one of the catalytic nuclease domains causing the Cas protein to nick or enzymatically break only one of the two DNA strands using the remaining active nuclease domain. For example, Wild-type Cas9 has two catalytic nuclease domains facilitating double-stranded DNA breaks and Cas9 nickases are known in the art (see, e.g., U.S. Patent Application Publication 2017/0051312, incorporated herein by reference) and include, for example, Streptococcus pyogenes with point mutations at D10 or H840. In select embodiments, the Cas9 nickase is Streptococcus pyogenes Cas9n (D10A).

In some embodiments, the Cas protein is a catalytically dead Cas. For example, catalytically dead Cas9 is essentially a DNA-binding protein due to, typically, two or more mutations within its catalytic nuclease domains which renders the protein with very little or no catalytic nuclease activity. Streptococcus pyogenes Cas9 may be rendered catalytically dead by mutations of D10 and at least one of E762, H840, N854, N863, or D986, typically H840 and/or N863A (see, e.g., U.S. Patent Application Publication 2017/0051312, incorporated herein by reference). Mutations in corresponding orthologs are known, such as N580 in Staphylococcus aureus Cas9. Oftentimes, such mutations cause catalytically dead Cas proteins to possess no more than 3% of the normal nuclease activity.

The present compositions may further include at least one unfoldase protein. Unfoldases are proteins that catalyze the unfolding of a native protein without affecting the primary structure. The unfoldase may be an NTP driven unfoldase. NTP driven unfoldases may include ATP-dependent proteases, including, but not limited to, ATPases, AAA proteases, or AAA+ enzymes (e.g., AAA+ enzyme). In some embodiments, the at least one unfoldase protein may comprise ClpX (caseinolytic mitochondrial matrix peptidase chaperone subunit X). In some embodiments, the at least one unfoldase protein may comprise a homolog of ClpX.

ClpX homologs may be readily screened through systematic testing and optimization of a large panel of homologs, identified through bioinformatic search strategies such as BLASTp and psi-BLASTp. In some embodiments, the unfoldase protein (e.g., ClpX) is derived from the same host organism as that of the engineered proteins described above. In some embodiments, the unfoldase protein (e.g., ClpX) is derived from a different host organism as that of the engineered proteins described above. As such, the at least one unfoldase protein (e.g., ClpX) is not limited from which organism it is derived. In some embodiments, the unfoldase protein (e.g., ClpX) is derived from the E. coli genome. In other embodiments, the unfoldase protein (e.g., ClpX) from the cognate strain from which the engineered proteins described above are derived. For example, the unfoldase protein from Vibrio cholerae HE-45 can be used alongside RNA-guided DNA integration machinery derived from Tn6677, while unfoldase proteins from Pseudoalteromonas sp. S983 can be used alongside RNA-guided DNA integration machinery derived from Tn7016.

In some embodiments, the compositions further comprise one or more additional genome engineering tools. For example, the compositions may further comprise nucleases, such as zinc finger nucleases (ZFNs) and/or transcription activator like effector nucleases (TALENs); transcriptional activators, transcriptional repressors, histone-modifying proteins, integrases, and recombinases.

### Systems

Disclosed herein are systems for DNA integration into a target nucleic acid sequence comprising: an engineered Clustered Regularly Interspaced Short Palindromic Repeats (CRISPR)-associated transposon (CRISPR-Tn or CAST) system or one or more nucleic acids encoding the engineered CRISPR-Tn system. The CRISPR-Tn system comprises at least one or both of: a) one or more Cas proteins selected from: Cas5, Cas6, Cas7, Cas8, Cas9, Cas11, or Cas12; and b) one or more transposon-associated proteins selected from TnsA, TnsB, TnsC, TnsD, and TniQ.

The system may comprise one or more of the modified transposon-associated proteins and Cas proteins disclosed herein. In some embodiments, at least one of the one or more Cas protein comprises: a Cas6 protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 10 or 14 with one or more amino acid substitutions, deletions, or additions relative to SEQ ID NO: 10 or 14; a Cas7 protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 9 or 13 with one or more amino acid substitutions, deletions, or additions relative to SEQ ID NO: 9 or 13; or a Cas8 protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 8 or 12 with one or more amino acid substitutions, deletions, or additions relative to SEQ ID NO: 8 or 12. In some embodiments, at least one of the one or more transposon-associated proteins comprises: a TnsA protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 1 or 4 with one or more amino acid substitutions, deletions, or additions relative to SEQ ID NO: 1 or 4; a TnsB protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 2 or 5 with one or more amino acid substitutions, deletions, or additions relative to SEQ ID NO: 2 or 5; a TnsC protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 3 or 6 with one or more amino acid substitutions, deletions, or additions relative to SEQ ID NO: 3 or 6, or a TniQ protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 7 or 11 with one or more amino acid substitutions, deletions, or additions relative to SEQ ID NO: 7 or 11.

The system may comprise a modified transposon-associated protein and one or more modified Cas proteins. In some embodiments, the system comprises a TniQ protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 7 with one or more amino acid substitutions, deletions, or additions relative to SEQ ID NO: 7; and one or more of: a Cas6 protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 10 with one or more amino acid substitutions, deletions, or additions relative to SEQ ID NO: 10; a Cas7 protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 9 with one or more amino acid substitutions, deletions, or additions relative to SEQ ID NO: 9; or a Cas8 protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: with one or more amino acid substitutions, deletions, or additions relative to SEQ ID NO: 8.

In some embodiments, the TniQ protein comprises an amino acid sequence having one or more amino acid substitutions at positions: 99, 133, 189, 265, 266, 336, and 343, relative to SEQ ID NO: 7. In some embodiments, the TniQ protein comprises an amino acid sequence having one or more amino acid substitutions of: M99I, S189N, H265Q, A266V, L336F, and V343A, relative to SEQ ID NO: 7.

In some embodiments, the Cas6 protein comprises an amino acid having one or more amino acid substitutions at positions: 21 and 90, relative to SEQ ID NO: 10. In some embodiments, the Cas6 protein comprises an amino acid sequence having one or more amino acid substitutions of: A21S and V90A, relative to SEQ ID NO: 10.

In some embodiments, the Cas7 protein comprises an amino acid having one or more amino acid substitutions at positions: 28, 82, 144, 151, 162, 182, 273, 327, 346, relative to SEQ ID NO: 9. In some embodiments, the Cas7 protein comprises an amino acid sequence having one or more amino acid substitutions of: R28K, A82T, K144, C151R, N162S, K182E, D273G, A327D, and M346I, relative to SEQ ID NO: 9.

In some embodiments, the Cas8 protein comprises an amino acid having one or more amino acid substitutions at positions: 119, 134, 155, 180, 183, 274, 319, 447, 454, 458, 461, 512, 538, and 580, relative to SEQ ID NO: 8. In some embodiments, the Cas8 protein comprises an amino acid sequence having one or more amino acid substitutions of: Y119H, N134R, N134Q, D155N, Q180R, D183N, R274L, N319D, V447I, A454S, E458G, D461N, A512T, D538K, and P580Q, relative to SEQ ID NO: 8.

In some embodiments, the system comprises a TniQ protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 11 with one or more amino acid substitutions, deletions, or additions relative to SEQ ID NO: 11; and one or more of: a Cas6 protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 14 with one or more amino acid substitutions, deletions, or additions relative to SEQ ID NO: 14; a Cas7 protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 13 with one or more amino acid substitutions, deletions, or additions relative to SEQ ID NO: 13; and a Cas8 protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 12 with one or more amino acid substitutions, deletions, or additions relative to SEQ ID NO: 12.

In some embodiments, the TniQ protein comprises an amino acid sequence having one or more amino acid substitutions at positions: 2, 3, 7, 9, 11, 12, 14, 16, 20, 26, 29, 32, 34, 35, 40, 43, 45, 46, 54, 61, 64, 65, 70, 77, 101, 103, 105, 106, 108, 109, 111, 119, 120, 123, 126, 127, 130, 131, 148, 149, 151, 157, 159, 164, 166, 185, 194, 196, 203, 211, 217, 218, 219, 236, 242, 257, 267, 279, 283, 286, 288, 291, 293, 296, 303, 306, 313, 314, 316, 326, 331, 336, 347, 352, 361, 374, 377, 395, 396, 398, and 408, relative to SEQ ID NO: 11. In some embodiments, the TniQ protein comprises an amino acid sequence having one or more amino acid substitutions of: A2T, F3S, P7R, A9S, A9G, A11G, F12I, D14N, S16Y, Y20H, S26N, F29S, S32N, E34K, G35V, G35S, G35D, I40S, E43D, H45P, E46K, A54S, R61W, V64M, Y65C, N70S, A77T, D101N, K103E, N105K, N105D, S106G, V108M, A109G, Y111N, L119M, R120S, R123S, A126T, E127G, V130M, D131N, Q148R, S149Y, H151Y, A157D, T159I, A164V, L166M, T185A, S194G, A196T, T203A, K211R, E217K, R218K, R218S, N219S, A236T, E242D, N257K, N267S, M279I, M279V, D283G, N286S, T288I, K291Q, I293V, D296N, S303I, S303G, K306N, S310Y, S310P, I313T, Y314F, A316T, E326G, T331I, A336V, A347T, A347S, T352S, Y361H, M374T, M374I, R377G, T395I, S396T, S396F, G398V, and A408V, relative to SEQ ID NO: 11. In some embodiments, the TniQ protein comprises an amino acid sequence having one or more amino acid substitutions at positions: 105, 109, 131, 148, 279, and 310; or 9, 105, 109, 131, 148, 279, and 310, relative to SEQ ID NO: 11. In some embodiments, the TniQ protein comprises an amino acid sequence having one or more amino acid substitutions of: N105K, A109G, D131N, Q148R, M279I, and S310Y or S310P, relative to SEQ ID NO: 11. In some embodiments, the TniQ protein comprises an amino acid sequence having one or more amino acid substitutions of: A9S, N105K, A109G, D131N, A148R, M2791, and S310P, relative to SEQ ID NO: 11.

In some embodiments, the Cas6 protein comprises an amino acid sequence having one or more amino acid substitutions at positions: 2, 9, 13, 14, 15, 34, 38, 42, 46, 50, 59, 60, 73, 75, 77, 82, 83, 85, 86, 97, 110, 115, 120, 124, 130, 132, 134, 140, 143, 145, 156, 159, 162, 164, 177, 199, 232, and 270, relative to SEQ ID NO: 14. In some embodiments, the Cas6 protein comprises an amino acid sequence having one or more amino acid substitutions of: Q2K, H9L, K13E, Q14K, A15G, K34N, E38K, V42I, A46D, S50I, V59G, Y60H, A73S, A73T, F75L, D77G, G82S, F83L, F83V, F83C, K85E, V86I, E97, I110S, I110L, S115R, K120N, K124R, G130D, D132E, N134T, A140T, E143K, D145G, S156I, E159K, I162V, H164Y, H164F, Y177C, S199I, S232L, and L270S, relative to SEQ ID NO: 14.

In some embodiments, the Cas6 protein comprises an amino acid sequence having one or more amino acid substitutions at positions: 82, 110, 115, 164, and 199, relative to SEQ ID NO: 14. In some embodiments, the Cas6 protein comprises an amino acid sequence having one or more amino acid substitutions of: G82S, I110S, S115R, H164Y or H164F, and S199I, relative to SEQ ID NO: 14. In some embodiments, the Cas6 protein comprises an amino acid sequence having one or more amino acid substitutions at positions: 110, 115, 164, 199, and 124, relative to SEQ ID NO: 14. In some embodiments, the Cas6 protein comprises an amino acid sequence having one or more amino acid substitutions of: I110S, S115R, K124R, H164Y or H164F, and S199I, relative to SEQ ID NO: 14. In some embodiments, the Cas6 protein comprises an amino acid sequence having one or more amino acid substitutions at positions: 82, 110, 115, 124, 164, and 199, relative to SEQ ID NO: 14. In some embodiments, the Cas6 protein comprises an amino acid sequence having one or more amino acid substitutions of: G82S, I110S, S115R, K124R, H164Y, and S199I, relative to SEQ ID NO: 14. In some embodiments, the Cas6 protein comprises an amino acid sequence having one or more amino acid substitutions at positions: 110, 115, and 164, relative to SEQ ID NO: 14. In some embodiments, the Cas6 protein comprises an amino acid sequence having one or more amino acid substitutions of: I110S, S115R, and H164F, relative to SEQ ID NO: 14. In some embodiments, the Cas6 protein comprises an amino acid sequence having one or more amino acid substitutions at positions: 110, 115, 164, and 199, relative to SEQ ID NO: 14. In some embodiments, the Cas6 protein comprises an amino acid sequence having one or more amino acid substitutions of: I110S, S115R, H164F, and S199I, relative to SEQ ID NO: 14. In some embodiments, the Cas6 protein comprises an amino acid sequence having one or more amino acid substitutions at positions: 110, 115, 164, 199, and 124, relative to SEQ ID NO: 14. In some embodiments, the Cas6 protein comprises an amino acid sequence having one or more amino acid substitutions of: I110S, S115R, H164F, S199I, and K124R, relative to SEQ ID NO: 14.

In some embodiments, the Cas7 protein comprises an amino acid sequence having one or more amino acid substitutions at positions: 5, 10, 11, 26, 30, 35, 40, 42, 45, 46, 47, 58, 61, 65, 71, 72, 75, 77, 78, 80, 82, 83, 94, 98, 113, 115, 116, 117, 121, 128, 133, 138, 146, 148, 161, 171, 175, 177, 182, 184, 191, 193, 201, 203, 211, 212, 219, 225, 226, 232, 233, 235, 236, 237, 238, 240, 250, 274, 282, 286, 292, 295, 304, 307, 309, 312, 313, 315, 316, 317, 318, 320, 321, 322, 323, 328, 340, 343, 344, 345, 347, 348, 349, and 350, relative to SEQ ID NO: 13. In some embodiments, the Cas7 protein comprises an amino acid sequence having one or more amino acid substitutions of: N5K, N5T, DION, R11K, D26N, V30E, D35N, R40L, P42A, G45S, G45V, F46V, T47R, T47S, N58T, P61L, T65I, T71I, T71R, T71D, L72M, C75S, V77A, P78L, N80T, E82D, H83Y, H83N, A94S, V98M, E113D, C121F, A128S, A155S, E116D, T117I, R133K, G138V, N146D, G148V, C161R, A171V, A171S, K175T, A177V, K182E, L184M, I191V, S193A, S193F, F201S, S203N, E211K, A212V, Y219R, N225S, N225T, D226Y, E232K, E232Q, A233N, A233S, A233K, K235R, Q236R, Q236S, F237L, V238Q, V238M, A240T, A240V, S250A, R274G, A282V, I286N, I286T, I286F, P292S, S295N, K304R, E307D, Y309C, A312V, L313M, N315K, N315T, N315S, C316G, I317V, T318A, T318P, K320R, N321D, E322K, K323N, I328T, M340I, K343E, K343R, K344E, K344R, A345T, A345D, A345S, A345Y, A345R, A345K, A345E, A345G, A347K, A347S, A347D, K348N, K349R, A350K, A350D, A350V, and A350T, relative to SEQ ID NO: 13.

In some embodiments, the Cas7 protein comprises an amino acid sequence having amino acid substitutions at positions: 30, 46, 240, 304, and 316; 30, 46, 240, and 316; 42 and 318; 184, 240, 315, and 345; 211 and 274; 237 and 237; 286 and 350; 317 and 347; 171, 286, and 315; or 328 and 350, relative to SEQ ID NO: 13. In some embodiments, the Cas7 protein comprises an amino acid sequence having amino acid substitutions of: V30E, F46V, A240T or A240V, K304R, and C316G, relative to SEQ ID NO: 13. In some embodiments, the Cas7 protein comprises an amino acid sequence having amino acid substitutions of: V30E, F46V, A240T or A240V, and C316G, relative to SEQ ID NO: 13. In some embodiments, the Cas7 protein comprises an amino acid sequence having amino acid substitutions of: L184M, A240T or A240V, N315K, and A345T, relative to SEQ ID NO: 13. In some embodiments, the Cas7 protein comprises an amino acid sequence having amino acid substitutions of: 1286N and A350D, relative to SEQ ID NO: 13. In some embodiments, the Cas7 protein comprises an amino acid sequence having amino acid substitutions of: A171S, I286F, and N315S, relative to SEQ ID NO: 13.

In some embodiments, the Cas7 protein comprises an amino acid sequence having at least 70% identity to SEQ ID NO: 13 and at least one amino acid substitution with a positively charged amino acid. In some embodiments, the positively charged amino acid is arginine. In some embodiments, the at least one amino acid substitution is at position 2, 5, 6, 7, 8, 9, 10, 12, 38, 39, 40, 41, 42, 43, 44, 45, 46, 47, 48, 64, 65, 66, 67, 68, 69, 70, 222, 224, 225, 227, 228, 229, 231, 232, 233, 234, 235, 255, 256, 257, 258, 277, 286, 287, 337, 338, 339, 340, 345, 346, 347, 348, 349, 350, or a combination thereof, relative to SEQ ID NO: 13. In some embodiments, the at least one amino acid substitution is at positions: 346 and 348; 346, 348 and 349; 346, 348, 349, an 350; 350 and 351; 350, 351, and 352; 350, 351, 352, and 353; 235 and 227; 235 and 345; 235 and 346; 235 and 347; 235 and 348; 235 and 349; 235 and 350; 235, 227, and 349; 5, 235 and 346; 5, 235 and 348; 5, 235 and 349; 227, 235, and 346; or 227, 235, and 348, relative to SEQ ID NO: 13.

In some embodiments, the Cas8 protein comprises an amino acid sequence having one or more amino acid substitutions at positions: 4, 5, 6, 8, 9, 11, 12, 13, 16, 17, 20, 21, 24, 26, 28, 29, 34, 37, 38, 41, 49, 54, 59, 60, 63, 65, 67, 74, 77, 81, 88, 92, 93, 94, 96, 102, 105, 106, 108, 110, 121, 126, 128, 134, 138, 142, 147, 150, 151, 153, 156, 157, 160, 162, 165, 170, 171, 173, 174, 179, 181, 183, 185, 186, 187, 188, 191, 198, 201, 206, 207, 226, 228, 233, 236, 241, 249, 250, 256, 267, 268, 270, 275, 276, 277, 279, 283, 286, 289, 303, 305, 306, 310, 312, 314, 315, 316, 323, 326, 329, 349, 353, 355, 356, 357, 358, 361, 370, 372, 373, 376, 378, 382, 388, 391, 397, 399, 403, 405, 419, 421, 423, 424, 425, 427, 428, 430, 431, 432, 433, 449, 457, 473, 477, 480, 485, 487, 489, 494, 496, 497, 498, 500, 502, 509, 511, 515, 518, 519, 520, 540, 545, 550, 555, 557, 570, 571, 580, 583, 585, 590, 594, 603, 607, 608, 611, 617, 620, 624, 636, 639, 641, 642, 644, 646, 655, 658, 660, 663, 665, 668, 672, 673, 678, 682, 685, 688, and 695, relative to SEQ ID NO: 12. In some embodiments, the Cas8 protein comprises an amino acid sequence having one or more amino acid substitutions of: K4N, E5K, L6M, L6I, E8K, E8D, 19T, D11N, T12A, T13I, D16G, R17C, R17S, R20K, R20E, R21E, R21K, S24K, S24Q, S24R, Y26S, Y26H, A28S, A28D, M29I, G34D, A37S, V38M, V38G, I41V, R49L, D54G, K59R, K60N, K63N, A65T, A65V, K67E, K74E, K77E, W81C, K88R, K88E, 192T, R93E, R93K, V94M, K96N, E102D, E102G, T105A, L106M, S108P, V110A, G121S, S126P, K128R, L134M, Y138S, Q142H, W147L, K150N, V151M, V151L, A153T, S156R, S156G, D157N, K160R, K160E, A162T, S165N, S165G, V170E, K171E, F173V, K174N, K174R, T179A, K181T, S183N, P185T, E186K, E186D, E187K, A188S, A188V, D191Y, D191E, R198H, R198C, R198S, R201K, D206G, G207D, A226T, I228V, R233K, N236T, R241E, A249S, A250S, I256T, S267G, S267N, K268N, H270P, S275N, S275G, R276G, A277D, A277S, A277T, K279N, G283D, V286G, V289M, G303D, I305T, F306S, A310D, A310T, A312G, A312D, A312T, K314N, Q315R, R316G, N323S, E326A, E326K, N329S, G349D, E353D, L355M, L355R, E356G, E356D, S357P, A358V, R361S, P370T, N372K, E373D, S376F, T378I, F382L, M388V, G391S, R397K, A399S, K403N, M405I, L419P, D421N, K423R, H424N, H424R, V425L, I427V, E428K, D430A, D431G, E432D, H433N, A449T, G457D, R473K, E477D, G480D, F485L, S487R, S487G, S489N, N494D, S496N, A497S, V498G, K500N, K502N, Q509R, A511T, A511E, R515S, R518S, P519T, G520D, G520V, Y540C, Q545H, K550N, K555E, H557Q, P570S, E571D, C580R, S583R, E585K, E585G, E590D, R594K, M603I, H607N, H607L, K608R, D611N, L617P, N620S, K624N, T636P, M639V, N641S, V642G, S644N, S644G, E646D, A655V, V658M, K660N, T663A, T665I, R668S, I672V, G673V, S678R, M682L, A685V, A685D, K688N, and V695M, relative to SEQ ID NO: 12.

In some embodiments, the Cas8 protein comprises an amino acid sequence having amino acid substitutions at positions: 134, 179, 185, 540, 555, 624, and 646; 138, 250, 275, and 421; 303, 405, 520, and 590; 134, 179, 185, 540, 555, and 646, 4 and 49; 4 and 388; 4 and 571; 4, 162, and 480; 4 and 315; 5 and 316; 17 and 156; 38, 108, 497 and 583; 59, 157 and 644; 96, 305, 550 and 642; 106, 160 and 228; 312, 424, 449 and 457; or 376 and 611, relative to SEQ ID NO: 12. In some embodiments, the Cas8 protein comprises an amino acid sequence having amino acid substitutions of: L134M, T179A, P185T, Y540C, K555E, K624N, and E646D, relative to SEQ ID NO: 12. In some embodiments, the Cas8 protein comprises an amino acid sequence having amino acid substitutions of: Y138S, A250S, S275N, and D421N, relative to SEQ ID NO: 12. In some embodiments, the Cas8 protein comprises an amino acid sequence having amino acid substitutions of: L134M, T179A, P185T, Y540C, K555E, and E646D, relative to SEQ ID NO: 12. In some embodiments, the Cas8 protein comprises an amino acid sequence having amino acid substitutions of: G303D, M405I, G520D, and E590D, relative to SEQ ID NO: 12.

In some embodiments the systems comprise one or more of Cas6, Cas7, Cas8, and TniQ proteins having one or more amino acid substitutions, deletions, or additions relative to SEQ ID NOs: 7-14, as shown in Tables 3 and 4.

In some embodiments, the systems comprise TnsA and TnsB.

In some embodiments, the system comprises a TnsA protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 1 with one or more amino acid substitutions, deletions, or additions relative to SEQ ID NO: 1 and/or a TnsB protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 2 with one or more amino acid substitutions, deletions, or additions relative to SEQ ID NO: 2.

In some embodiments, the TnsA protein comprises an amino acid sequence having one or more amino acid substitutions at positions: 2, 3, 5, 28, 57, 77, 80, 107, 110, 116, 122, 142, 155, 161, 166, 173, 177, 185, 211, 216, 227, and 230, relative to SEQ ID NO: 1. In some embodiments, the TnsA protein comprises an amino acid sequence having one or more amino acid substitutions of: A2T, T3I, L5S, T28A, A57T, F77L, Y80D, K107M, K107R, Y110C, Y110D, D116G, E122A, D142E, M155I, K161R, N166D, K173E, Y177N, Y177D, C185R, D211Y, K216E, A227P, G230D and G230S, relative to SEQ ID NO: 1.

In some embodiments, the TnsA protein comprises an amino acid sequence having amino acid substitutions at positions: 2 and 230; 107 and 166; 107, 166, and one or both of: 2 and 227; 211 and 110 or 142; 110, 155 and 230; 122 and 155; 155 and 177, relative to SEQ ID NO: 1. In some embodiments, the TnsA protein comprises an amino acid sequence having amino acid substitutions of: A2T, and G230D or G230S, relative to SEQ ID NO: 1. In some embodiments, the TnsA protein comprises an amino acid sequence having amino acid substitutions of: K107M and N166D, relative to SEQ ID NO: 1. In some embodiments, the TnsA protein further comprises amino acid substitutions of: A2T and/or Y177N or Y177D, relative to SEQ ID NO: 1. In some embodiments, the TnsA protein comprises an amino acid sequence having amino acid substitutions of: D211Y and Y110C, Y110D, or D142E, relative to SEQ ID NO: 1. In some embodiments, the TnsA protein comprises an amino acid sequence having amino acid substitutions of: Y110C or Y110D, M155I, and G230D or G230S, relative to SEQ ID NO: 1. In some embodiments, the TnsA protein comprises an amino acid sequence having amino acid substitutions of: E122A and M155I, relative to SEQ ID NO: 1. In some embodiments, the TnsA protein comprises an amino acid sequence having amino acid substitutions of: M155I and Y177N or Y177D, relative to SEQ ID NO: 1.

In some embodiments, the TnsB protein comprises an amino acid sequence having one or more amino acid substitutions at positions: 2, 5, 22, 24, 25, 29, 75, 141, 199, 215, 319, 347, 364, 370, 383, 439, 454, 458, 485, 509, 533, 538, 565, 581, 586, 595, 596, 597, and 600, relative to SEQ ID NO: 2. In some embodiments, the TnsB protein comprises an amino acid sequence having one or more amino acid substitutions of: A2T, A2S, G5R, S22P, E24D, L25I, A29S, P75T, I141T, V199I, S215R, D319V, Y347F, S364N, E370K, N383D, V439A, E454D, E454G, S458N, V485F, R509G, D533A, A538V, H565Y, A581T, H586L, N595K, D596N, D597N, D597Y, and I600V, relative to SEQ ID NO: 2

In some embodiments, the TnsB protein comprises an amino acid sequence having amino acid substitutions at positions: 2 and 597; 24 and 25; 24, 25, 458, 509, 565, and 600; 75 and 597; 141, 454, 533 and 595; 581, 370, and 454; 370 and 581; 370 and 454; 458 and 509; 458, 509 and 565; 458, 509, 565, and 600; 565, 586, and 596; or 565, 509, 458, 600 and at least one of 24, 25, 29, 215, 319, 364, 383, and 586, relative to SEQ ID NO: 2.

In some embodiments, the TnsB protein comprises an amino acid sequence having amino acid substitutions of: A2T or A2S, and D597N or D597Y, relative to SEQ ID NO: 2. In some embodiments, the TnsB protein comprises an amino acid sequence having amino acid substitutions of: E24D and L25I, relative to SEQ ID NO: 2. In some embodiments, the TnsB protein comprises an amino acid sequence having amino acid substitutions of: E24D and L25I, S458N, R509G, H565Y, and I600V, relative to SEQ ID NO: 2. In some embodiments, the TnsB protein comprises an amino acid sequence having amino acid substitutions of: P75T and D597N or D597Y, relative to SEQ ID NO: 2. In some embodiments, the TnsB protein comprises an amino acid sequence having amino acid substitutions of: E454D or E454G, D533A, N595K, relative to SEQ ID NO: 2. In some embodiments, the TnsB protein comprises an amino acid sequence having amino acid substitutions of: E370K, E454D or E454G, and A581T, relative to SEQ ID NO: 2. In some embodiments, the TnsB protein comprises an amino acid sequence having amino acid substitutions of: E370K, and A581T, E454D, or E454G, relative to SEQ ID NO: 2. In some embodiments, the TnsB protein comprises an amino acid sequence having amino acid substitutions of: S458N and R509G, relative to SEQ ID NO: 2. In some embodiments, the TnsB protein further comprises amino acid substitutions of: H565Y and/or I600V. In some embodiments, the TnsB protein comprises an amino acid sequence having amino acid substitutions of: H565Y, H586L, and D596N, relative to SEQ ID NO: 2. In some embodiments, the TnsB protein comprises amino acid substitutions of: H565Y, R509G, S458N, I600V and at least one of E24D, L25I, A29S, S215R, D319V, S364N, N383D, and H586L, relative to SEQ ID NO: 2.

In some embodiments, the system further comprises a TnsC protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 3 with one or more amino acid substitutions, deletions, or additions relative to SEQ ID NO: 3. In some embodiments, the TnsC protein comprises an amino acid sequence having one or more amino acid substitutions at positions: 9, 15, 16, 18, 21, 64, 81, 86, 87, 99, 109, 142, 147, 153, 168, 180, 216, 230, 285, and 304, relative to SEQ ID NO: 3. In some embodiments, the TnsC protein comprises an amino acid sequence having one or more amino acid substitutions of: 19V, A15V, F16Y, S18F, S21N, N64D, H81Y, D86Y, N87K, V99I, E109D, E142K, V147I, N153D, I168M, A180E, A216S, L230F, K285E, and R304R, relative to SEQ ID NO: 3. In some embodiments, the TnsC protein comprises an amino acid sequence having amino acid substitutions at positions: 142 and 216, relative to SEQ ID NO: 3. In some embodiments, the TnsC protein comprises an amino acid sequence having amino acid substitutions of: E142K and A216S, relative to SEQ ID NO: 3.

In some embodiments the systems comprise one or more of TnsA, TnsB, and TnsC proteins having one or more amino acid substitutions, deletions, or additions relative to SEQ ID NOs: 1-3, as shown in Table 1.

In some embodiments, the system comprises a TnsA protein comprising an amino acid sequence having SEQ ID NO: 4. In some embodiments, the system comprises a TnsA protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 4 with one or more amino acid substitutions, deletions, or additions relative to SEQ ID NO: 4 and/or a TnsB protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 5 with one or more amino acid substitutions, deletions, or additions relative to SEQ ID NO: 5.

In some embodiments, the TnsA protein comprises an amino acid sequence having one or more amino acid substitutions at positions: 4, 5, 9, 10, 12, 21, 23, 25, 26, 31, 32, 34, 35, 37, 41, 45, 47, 48, 51, 52, 55, 60, 61, 65, 67, 69, 72, 75, 79, 80, 82, 87, 88, 90, 91, 93, 94, 96, 98, 99, 100, 103, 106, 108, 113, 116, 125, 126, 128, 129, 135, 139, 143, 146, 147, 149, 153, 154, 156, 158, 159, 160, 162, 164, 166, 167, 168, 169, 170, 177, 179, 180, 182, 183, 185, 187, 188, 190, 191, 192, 193, 195, 196, 200, 204, 207, and 208, relative to SEQ ID NO: 4. In some embodiments, the TnsA protein comprises an amino acid sequence having one or more amino acid substitutions of: R4K, N5K, P9S, A10P, N12D, T21I, V23M, S25N, S25R, V26M, V26G, S31N, S321, E34A, F35L, A37D, H41L, D45N, I47V, E48G, G51V, S52I, E55K, E55D, E60K, F61L, S65T, S65A, P67T, P67L, P67S, P67H, T69A, A72V, A72D, S75I, S75R, S75T, K79E, T80P, K82E, K87R, P88L, P88T, P88A, S90F, K91N, K91E, A93T, A93S, S94N, L96P, R98Q, A99D, A99V, E100K, A103T, A106T, S108A, 1113F, V116F, V116I, V125M, V125A, N126T, I128V, I128L, L129P, L135M, S139N, S139G, G143V, G143C, G146D, G146S, I147V, K149E, K149T, K149R, S153I, S153R, S153N, F154C, H156R, H156L, S158N, S158R, G159V, V160A, K162R, N164D, I166L, S167I, S168I, S168R, S168N, Q169R, V170M, V170G, V170L, T177I, T177A, S179R, F180C, F180L, F182C, F182L, G183S, M185I, K187R, G188D, V190I, K191N, A192S, D193N, G195V, G195D, G195S, C196W, T200A, T204I, A207V, A207T, and T208I, relative to SEQ ID NO: 4.

In some embodiments, the TnsA protein comprises an amino acid sequence having amino acid substitutions at positions: 108 and 47 or 208; 170 and 207; 88 and 147; 47, 88 and 147; 88, 147, 170, and 182; 88, 147, 170, 182, and 51 or 180; 88, 147, and 154; 88, 128, 147, 170, and 182; or 170, 207, and 108, relative to SEQ ID NO: 4. In some embodiments, the TnsA protein comprises an amino acid sequence having amino acid substitutions of: S108A, and I47V or T208I, relative to SEQ ID NO: 4. In some embodiments, the TnsA protein comprises an amino acid sequence having amino acid substitutions of: V170M, and A207V or A207T, relative to SEQ ID NO: 4. In some embodiments, the TnsA protein further comprises amino acid substitutions of: S108A, V170M, and A207V or A207T, relative to SEQ ID NO: 4. In some embodiments, the TnsA protein further comprises amino acid substitutions of: P88T, I147V, V170L, and F182L, relative to SEQ ID NO: 4. In some embodiments, the TnsA protein further comprises amino acid substitutions of: P88T, I147V, V170L, F182L and G51V or F180L, relative to SEQ ID NO: 4. In some embodiments, the TnsA protein further comprises amino acid substitutions of: P88T, I147V, and F154C, relative to SEQ ID NO: 4.

In some embodiments, the TnsB protein comprises an amino acid sequence having one or more amino acid substitutions at positions: 1, 2, 4, 5, 8, 9, 10, 11, 12, 13, 14, 15, 16, 17, 19, 20, 21, 22, 23, 24, 25, 26, 27, 28, 29, 30, 32, 33, 36, 37, 39, 40, 41, 42, 43, 44, 45, 49, 52, 55, 56, 58, 60, 62, 63, 67, 71, 74, 76, 78, 79, 80, 81, 82, 83, 84, 85, 86, 87, 88, 89, 91, 92, 95, 97, 100, 101, 104, 106, 110, 112, 113, 115, 117, 119, 120, 124, 125, 127, 129, 130, 131, 134, 139, 142, 144, 145, 146, 147, 149, 150, 155, 156, 157, 158, 159, 163, 164, 165, 167, 169, 173, 174, 176, 181, 182, 186, 187, 190, 195, 197, 198, 205, 208, 209, 211, 215, 218, 223, 226, 227, 231, 232, 235, 239, 246, 248, 250, 259, 260, 261, 262, 263, 267, 269, 273, 274, 277, 278, 280, 281, 282, 283, 285, 287, 288, 290, 295, 298, 302, 303, 307, 313, 316, 317, 320, 323, 325, 331, 332, 339, 345, 348, 349, 352, 353, 354, 356, 361, 362, 363, 364, 365, 366, 367, 369, 370, 371, 372, 373, 375, 376, 380, 383, 385, 386, 389, 390, 392, 396, 397, 399, 402, 403, 404, 407, 408, 410, 411, 412, 413, 414, 415, 416, 421, 422, 423, 424, 425, 426, 427, 428, 429, 430, 431, 434, 435, 437, 440, 443, 445, 446, 448, 450, 452, 456, 459, 460, 463, 464, 470, 472, 473, 494, 495, 498, 501, 502, 504, 505, 506, 508, 509, 510, 512, 513, 514, 517, 520, 521, 522, 525, 526, 527, 530, 531, 532, 533, 535, 537, 538, 540, 541, 542, 543, 544, 545, 546, 547, 548, 549, 550, 551, 552, 553, 554, 556, 557, 558, 559, 560, 561, 562, 563, 564, 565, 567, 568, 569, 570, 571, 574, 575, 576, 580, 582, 583, 584, 585, 586, 587, 588, 589, 590, 591, 592, 593, 594, 595, 596, 597, 599, 600, 601, 602, 603, 604, 606, 607, 608, 611, 613, 618, 620, and 656, relative to SEQ ID NO: 5. In some embodiments, the TnsB protein comprises an amino acid sequence having one or more amino acid substitutions of: M1V, M1I, M1L, T2I, T2A, F4L, F5L, F8L, F8V, F8S, D9N, E10K, E10D, S11I, S11R, S11G, L12P, V13M, V13G, V13E, V13L, P14L, L15Q, K16N, K16R, P17T, P17L, P17S, T19I, T19S, T19A, T19P, P20S, P20L, T21A, Q22R, Y23H, V24M, K25R, L26M, D27A, D27G, D28N, D28Y, A29T, A29V, N30K, I32F, I32S, Q33H, L36M, D37A, D37Y, F39L, S40P, D41E, T42I, T42K, T42A, F43L, F43S, F43V, K44N, N45D, N45S, Q49R, K52Q, S55A, T56A, D58E, K60Q, S62T, R63K, R63G, Q67R, Q67H, Q67K, D71Y, K74R, E76K, F78C, K79R, G80V, G80D, G81S, G81V, G81D, D82N, V83G, V83M, V83A, V84A, V84G, R85G, R85K, P86L, N87S, R89C, V91G, V91A, A92V, A92T, R95K, K97R, E100D, S101A, D104V, A106D, A106T, D110N, N112H, H113Y, M115R, N117Y, T119A, N120D, N120K, N120S, G124V, D125N, D125E, K127R, F129L, D130N, K131M, E134D, E134G, A139S, A139T, P142S, 1144V, A145S, A145T, T146A, A147V, Q149R, Y150H, I155L, V156A, V156L, V156M, K157V, E158A, N159S, V163G, E164A, E164G, E164D, G165D, I167V, I169L, I169T, N173S, N173H, N173T, A174S, A174T, N176D, A181S, I182L, I182V, I182T, A186E, A186T, V187G, V187A, A190T, A190S, F195S, A197P, D198G, D198N, A205S, V208M, P209T, T211I, E215D, E218D, P223S, P223H, L226V, I227V, D231N, E232K, I235V, I235T, R239G, I246V, V248E, V248M, S2501, S259N, Y260C, K261R, S262N, P263L, S267N, A269V, T273I, T273N, H274Y, K277N, K277R, P278S, S280T, L281M, D282E, D282N, A283T, A283S, N285S, E287D, L288M, N290K, F295S, F298I, F298S, V302I, V303M, A307S, N313S, H316R, A317V, S320N, S320R, I323L, I325V, R331K, K332E, I339V, V345L, V345M, E348K, Y349H, Y349D, Y349N, Y349C, P352S, P352T, E353Q, E353D, L354M, G356S, N361D, I362V, I362T, L363P, L363T, L363M, E364G, K365R, E366G, E367G, K369N, K369E, K369M, P370S, E371K, V372M, D373G, I375V, M376I, T380P, T380A, E383K, E383D, F385L, H386Y, I389V, A390V, A390I, V392I, D396N, D396G, D396K, S397P, S399N, S399G, T402I, R403G, R403I, R403K, R403S, I404T, I404V, K407R, K407E, R408K, Q410K, Q410H, Q410R, Q411H, G412V, F413L, D414N, A415V, A415T, Y416C, M421I, N422K, E423K, E423D, E424A, E425K, E426D, T427A, T427S, R428K, F429L, S430A, M431L, R434H, R434C, R434S, I435V, D437G, D437N, T440S, T440I, R443C, G445S, F446L, F446I, Y448C, E450D, E450G, M4521, T456P, T456A, T456I, A459T, D460N, K463N, H464N, H464R, H464S, E470K, V472M, V472A, K473D, K473N, E494D, E494G, S495A, E498A, E498K, C501Y, T502I, T502S, P504S, P504L, T505A, G506Y, G506D, G506L, G506S, T508A, D509E, D509Y, C510Y, S512N, I513L, I513V, I513F, Y514H, K517M, K517N, K517Q, K520R, K521N, I522T, I522V, I522F, E525K, V526E, V526M, I527V, S530N, S530R, K531T, D532G, D532Y, S533Y, G535D, A537T, K538R, K538N, R540K, R540G, M541L, A542T, I543L, H544R, E545A, R546G, R546K, V547M, K548Q, K548R, Q549K, Q549R, E550A, Q551K, E552D, E552K, V553I, F554V, E556K, E556G, S557A, K558R, T559P, T559I, T559A, K560R, A561T, A561G, K562R, K562N, I563L, T564I, A565S, A565V, K567R, K568N, K568R, Q569K, Q569L, Q569R, A570V, Q571R, D574N, V575M, V575A, S576R, T580I, T580A, T582I, T582S, I583V, K584R, V585M, S586P, S586A, S586F, E587A, E588K, E588G, E588D, S589I, S589R, S589N, A590S, A590T, A591V, P592L, V593M, V593A, Q594L, K595R, K595N, H596Y, H596L, H596P, I597T, I597V, N599H, D600L, D600N, D600G, D600V, N601S, N601K, S602A, S602P, S602Y, D603A, D603V, D604G, D604Y, D604N, D606A, D606V, D606Y, D607Y, D607E, D608N, A611T, E613D, R618I, T620P, and A656V, relative to SEQ ID NO: 5.

In some embodiments, the TnsB protein comprises an amino acid sequence having amino acid substitutions at positions: 4, 23 and 590; 19, 169, and 549; 43 and 415; 80 and 593; 80, 144, 593, and 606; 1, 42, 80, 593, and 606; 42, 80, 593, and 606; 156 and 604; 283, 349, and 365; 283, 349, 365, 396, and 594; 283, 349, 365, 396, 594, 596, and 131; 352 and 390; 390, 396, and 594; 396 and 594; 456 and 502; 464 and 502; 464 and 17; 17, 235, 464, and 596; 235, 352, 396, 456, and 606; 415, 456, and 502; 456, 502, and 549; 169, 456, 502, and 549; 80, 456, 502, 593, and 606; 1, 42, 80, 456, 502, 593, and 606; 80, 144, 456, 502, 593, and 606; 19, 169, 456, 502 and 549; 43, 415, 456, and 502; 352, 390, 396, and 594; 352, 390, and 396; 283, 349, 396, and 594; 11, 55, 120, 362, 584, 600, and 604; 43, 84, 144, 349, and 517; 164 and 165; 164 and 173; 362 and 446; 352, 390, 396, 549, and 594; 352, 390, 396, 464, 549, and 594; 43, 349, 352, 390, 396, 464, 549, and 594; 43, 349, 352, 390, 396, 464, 549, 594 and one or more positions selected from 63, 145, 174, 182, 208, 410, 427, 456, 504, and 526; 43, 352, 390, 396, 464, 549, and 594; 43, 349, 352, 390, 396, 464, 549, 594, 410, 526, 415, and 502; 43, 349, 352, 390, 396, 464, 549, 594, 410, 526, 415, 502, and 21; 43, 349, 352, 390, 396, 464, 549, 594, 410, 526, 415, 502, and 67; 43, 349, 352, 390, 396, 464, 549, 594, 410, 526, 415, 502, 21, and 67; 43, 349, 352, 390, 396, 464, 549, 594, 410, 526, 174, 208, 427, 456, and 504; 43, 349, 352, 390, 396, 464, 549, 594, 410, 526, 415, 502, and 139; 43, 349, 352, 390, 396, 464, 549, 594, 410, 526, 415, 502, 339, and 446; 43, 349, 352, 390, 396, 464, 549, 594, 410, 526, 19, 460, 569, and 596; 43, 349, 352, 390, 396, 464, 549, 594, 410, 526, 460, 586, 588, and 608; 43, 349, 352, 390, 396, 464, 549, 594, 410, 526, and 460; 352, 390, 396, 549, 586, and 594; 63, 158, 352, 390, 396, 549, 586, and 594; 164, 165, 352, 363, 390, 396, 410, 549, 586, and 594; 164, 173, 352, 390, 396, 549, 586, and 594; 83, 352, 390, 396, 549, 586, and 594; 8, 43, 174, 349, 352, 390, 396, 427, 464, 549, and 594; or 283, 349, 365, 396, 594, 596, and 131, relative to SEQ ID NO: 5.

In select embodiments, the TnsB protein comprises amino acid substitutions at positions: 352, 390, 396, 594, or any combination thereof, relative to SEQ ID NO: 5.

In select embodiments, the TnsB protein comprises amino acid substitutions at positions: F43, Y349, P352, A390, D396, H464, Q549, Q594, and T456; F43, Y349, P352, A390, D396, H464, Q549, Q594, T456, and V526; F43, Y349, P352, A390, D396, H464, Q549, Q594, and P504; F43, Y349, P352, A390, D396, H464, Q549, Q594, and V526; F43, Y349, P352, A390, D396, H464, Q549, Q594, Q410, and V526; F43, Y349, P352, A390, D396, H464, Q549, Q594, A174, and T427; F43, Y349, P352, A390, D396, H464, Q549, Q594, and V208; or F43, Y349, P352, A390, D396, H464, Q549, Q594, R63, A145, 1182, and V526; F43, Y349, P352, A390, D396, H464, Q549, Q594, Q410, V526, A415, and T502; F43, Y349, P352, A390, D396, H464, Q549, Q594, Q410, V526, A415, T502, and T21; F43, Y349, P352, A390, D396, H464, Q549, Q594, Q410, V526, A415, T502, and Q67; F43, Y349, P352, A390, D396, H464, Q549, Q594, Q410, V526, A415, T502, T21, and Q67; F43, Y349, P352, A390, D396, H464, Q549, Q594, Q410, V526, A174, V208, T427, T456, and P504; F43, Y349, P352, A390, D396, H464, Q549, Q594, Q410, V526, A174, V208, T427, T456, and P504; F43, Y349, P352, A390, D396, H464, Q549, Q594, Q410, V526, A415, T502, and A139; F43, Y349, P352, A390, D396, H464, Q549, Q594, Q410, V526, A415, T502, 1339, and F446; F43, Y349, P352, A390, D396, H464, Q549, Q594, Q410, V526, T19, D460, Q569, and H596; F43, Y349, P352, A390, D396, H464, Q549, Q594, Q410, V526, D460, S586, E588, and D608; F43, Y349, P352, A390, D396, H464, Q549, Q594, Q410, V526, and D460, relative to SEQ ID NO: 5.

In some embodiments, the TnsB protein comprises an amino acid sequence having amino acid substitutions of: F4L, Y23H and A590S, relative to SEQ ID NO: 5. In some embodiments, the TnsB protein comprises an amino acid sequence having amino acid substitutions of: T19P, I169L, and 549, relative to SEQ ID NO: 5. In some embodiments, the TnsB protein comprises an amino acid sequence having amino acid substitutions of: F43L and A415V, relative to SEQ ID NO: 5. In some embodiments, the TnsB protein comprises an amino acid sequence having amino acid substitutions of: G80D and V593M or V593A, relative to SEQ ID NO: 5. In some embodiments, the TnsB protein comprises an amino acid sequence having amino acid substitutions of: G80D, 1144V, V593M or V593A, and D606A or D606V, relative to SEQ ID NO: 5. In some embodiments, the TnsB protein comprises an amino acid sequence having amino acid substitutions of: M1V, M1I or M1L, T421 or T42A, G80D, V593M or V593A, and D606A or D606V, relative to SEQ ID NO: 5. In some embodiments, the TnsB protein comprises an amino acid sequence having amino acid substitutions of: T421 or T42A, G80D, V593M or V593A, and D606A or D606V, relative to SEQ ID NO: 5. In some embodiments, the TnsB protein comprises an amino acid sequence having amino acid substitutions of: V156A or V156M, and D604G or D604N, relative to SEQ ID NO: 5. In some embodiments, the TnsB protein comprises an amino acid sequence having amino acid substitutions of: A283S or A283T, Y349H, and K365R, relative to SEQ ID NO: 5. In some embodiments, the TnsB protein further comprises amino acid substitutions of: D396K and Q594K or Q594L, relative to SEQ ID NO: 5. In some embodiments, the TnsB protein further comprises amino acid substitutions of: P352S or P352T, H596Y or H596L, and K131M, relative to SEQ ID NO: 5. In some embodiments, the TnsB protein comprises an amino acid sequence having amino acid substitutions of: P352S or P352T and A390V, relative to SEQ ID NO: 5. In some embodiments, the TnsB protein comprises an amino acid sequence having amino acid substitutions of: A390V, D396K, and Q594K or Q594L, relative to SEQ ID NO: 5. In some embodiments, the TnsB protein comprises an amino acid sequence having amino acid substitutions of: D396K and Q594K or Q594L, relative to SEQ ID NO: 5. In some embodiments, the TnsB protein comprises an amino acid sequence having amino acid substitutions of: T456P, T456I, or T456A and T502I, relative to SEQ ID NO: 5. In some embodiments, the TnsB protein comprises an amino acid sequence having amino acid substitutions of: H464N or H464R and T502I, relative to SEQ ID NO: 5. In some embodiments, the TnsB protein comprises an amino acid sequence having amino acid substitutions of: H464N or H464R and P17T, P17L, or P17S, relative to SEQ ID NO: 5. In some embodiments, the TnsB protein comprises an amino acid sequence having amino acid substitutions of: P17T, P17L, or P17S, I235V or I235T, H464N or H464R, and Q569K, Q569L or Q569R, relative to SEQ ID NO: 5. In some embodiments, the TnsB protein comprises an amino acid sequence having amino acid substitutions of: I235V or I235T, P352S or P352T, D396K, T456P, T456I, or T456A, and D606A or D606V, relative to SEQ ID NO: 5. In some embodiments, the TnsB protein comprises an amino acid sequence having amino acid substitutions of: A415V, T456P, and T502I, relative to SEQ ID NO: 5. In some embodiments, the TnsB protein comprises an amino acid sequence having amino acid substitutions of: T456P, T502I, and Q549K, relative to SEQ ID NO: 5. In some embodiments, the TnsB protein comprises an amino acid sequence having amino acid substitutions of: I169L, T456P, T502I, and Q549K, relative to SEQ ID NO: 5. In some embodiments, the TnsB protein comprises an amino acid sequence having amino acid substitutions of: G80D, T456P, T502I, V593M, and D606A, relative to SEQ ID NO: 5. In some embodiments, the TnsB protein comprises an amino acid sequence having amino acid substitutions of: M1V, T42I, G80D, T456P, T502I, V593M, and D606A, relative to SEQ ID NO: 5. In some embodiments, the TnsB protein comprises an amino acid sequence having amino acid substitutions of: G80D, 1144V, T456P, T502I, V593M, and D606A, relative to SEQ ID NO: 5. In some embodiments, the TnsB protein comprises an amino acid sequence having amino acid substitutions of: T19P, I169L, T456P, T502I, and Q549K, relative to SEQ ID NO: 5. In some embodiments, the TnsB protein comprises an amino acid sequence having amino acid substitutions of: F43L, A415V, T456P, and T502I, relative to SEQ ID NO: 5. In some embodiments, the TnsB protein comprises an amino acid sequence having amino acid substitutions of: P352T, A390V, D396N, and Q594L, relative to SEQ ID NO: 5. In some embodiments, the TnsB protein comprises an amino acid sequence having amino acid substitutions of: P352T, A390V, and D396N, relative to SEQ ID NO: 5. In some embodiments, the TnsB protein comprises an amino acid sequence having amino acid substitutions of: A283T, Y349H, D396N, and Q594L, relative to SEQ ID NO: 5. In some embodiments, the TnsB protein comprises an amino acid sequence having amino acid substitutions of: A283T, Y349H, P352S, K365R, D396N, Q594L, H596L, and K131M, relative to SEQ ID NO: 5. In select embodiments, the TnsB protein comprises an amino acid sequence having one or more amino acid substitutions of: P352T, A390V, D396N, and Q594L, relative to SEQ ID NO: 5.

In some embodiments, the TnsB protein comprises an amino acid sequence having amino acid substitutions of: F43S, Y349N, P352T, A390V, D396N, H464R, Q549R, Q594L, and T456I, relative to SEQ ID NO: 5. In some embodiments, the TnsB protein comprises an amino acid sequence having amino acid substitutions of: F43S, Y349N, P352T, A390V, D396N, H464R, Q549R, Q594L, T456P, and V526E, relative to SEQ ID NO: 5. In some embodiments, the TnsB protein comprises an amino acid sequence having amino acid substitutions of: F43S, Y349D, P352T, A390V, D396N, H464R, Q549R, Q594L, and P504S, relative to SEQ ID NO: 5. In some embodiments, the TnsB protein comprises an amino acid sequence having amino acid substitutions of: F43S, Y349N, P352T, A390V, D396N, H464R, Q549R, Q594L, and V526E, relative to SEQ ID NO: 5. In some embodiments, the TnsB protein comprises an amino acid sequence having amino acid substitutions of: F43S, Y349N, P352T, A390V, D396N, H464R, Q549R, Q594L, Q410K, and V526E, relative to SEQ ID NO: 5. In some embodiments, the TnsB protein comprises an amino acid sequence having amino acid substitutions of: F43S, Y349N, P352T, A390V, D396N, H464R, Q549R, Q594L, A174S, and T427S, relative to SEQ ID NO: 5. In some embodiments, the TnsB protein comprises an amino acid sequence having amino acid substitutions of: F43S, Y349N, P352T, A390V, D396N, H464R, Q549R, Q594L, and V208M, relative to SEQ ID NO: 5. In some embodiments, the TnsB protein comprises an amino acid sequence having amino acid substitutions of: F43S, Y349N, P352T, A390V, D396N, H464R, Q549R, Q594L, R63G, A145S, I182T, and V526E, relative to SEQ ID NO: 5.

In some embodiments, the TnsB protein comprises an amino acid sequence having amino acid substitutions of: F43S, Y349N or Y349D, P352T, A390V, D396N, H464R, Q549R, Q594L, A415V and T502I, relative to SEQ ID NO: 5. In some embodiments, the TnsB protein comprises an amino acid sequence having amino acid substitutions of: F43S, Y349N or Y349D, P352T, A390V, D396N, H464R, Q549R, Q594L, A415V, T502I, and T21A, relative to SEQ ID NO: 5. In some embodiments, the TnsB protein comprises an amino acid sequence having amino acid substitutions of: F43S, Y349N or Y349D, P352T, A390V, D396N, H464R, Q549R, Q594L, A415V, T502I, and Q67K, relative to SEQ ID NO: 5. In some embodiments, the TnsB protein comprises an amino acid sequence having amino acid substitutions of: F43S, Y349N or Y349D, P352T, A390V, D396N, H464R, Q549R, Q594L, A415V, T502I, T21A, and Q67K, relative to SEQ ID NO: 5.

In some embodiments, the system further comprises a TnsC protein. In some embodiments, the TnsC protein comprises an amino acid sequence having SEQ ID NO: 6. In some embodiments, the system further comprises a TnsC protein comprising an amino acid sequence having at least 70% (e.g., having at least 75%, at least 80%, at least 85%, at least 90%, at least 92%, at least 95%, at least 96%, at least 97%, at least 98%, or at least 99%) identity to SEQ ID NO: 6 with one or more amino acid substitutions, deletions, or additions relative to SEQ ID NO: 6. In some embodiments, the TnsC protein comprises an amino acid sequence having one or more amino acid substitutions at positions: 1, 2, 3, 5, 6, 7, 9, 11, 12, 14, 21, 22, 26, 27, 31, 35, 38, 43, 44, 46, 47, 54, 59, 60, 61, 64, 65, 67, 68, 71, 72, 74, 76, 79, 80, 81, 84, 89, 95, 102, 105, 109, 110, 111, 112, 113, 114, 116, 118, 119, 120, 123, 129, 130, 131, 132, 134, 142, 145, 146, 147, 148, 150, 154, 155, 166, 169, 178, 180, 181, 183, 184, 187, 190, 194, 197, 201, 204, 207, 209, 213, 219, 221, 225, 226, 227, 229, 232, 233, 234, 236, 238, 241, 246, 251, 252, 256, 257, 261, 263, 265, 267, 269, 271, 272, 274, 280, 281, 285, 286, 288, 291, 292, 296, 299, 301, 303, 304, 306, 307, 308, 310, 313, 314, 316, 317, 318, 319, 320, 323, 324, 326, 328, 330, 331, 332, 340, 341, 343, 344, 355, 412, 418, 427, 514, 1198, 1201, 1206, 1212, 1260, and 1282, relative to SEQ ID NO: 6. In some embodiments, TnsC protein does not include substitutions at one or more positions selected from: 6, 9, 28, 44, 64, 76, 80, 95, 110, 113, 114, 116, 118, 130, 132, 142, 155, 187, 190, 194, 221, 233, 234, 238, 261, 272, 280, 281, 299, 303, 304, 307, 308, 313, 316, and 328, relative to SEQ ID NO: 6.

In some embodiments, the TnsC protein comprises an amino acid sequence having one or more amino acid substitutions of: M1L, M1V, N2S, A3T, T5P, T5A, T5S, E6D, 17S, 17V, 19F, Q11R, L12M, N14D, N14S, M21I, H22P, H22Y, K26N, K26R, T27I, M31I, L35R, N38S, S43P, D44N, D44G, Q46L, C47S, T54I, S59T, H60Y, T61A, H64Y, Y65H, K67N, K67R, R68Q, A71G, T72A, N74D, S76C, S76Y, T791, M80I, P81S, V84L, R89L, A95D, A95T, A102T, E105D, E105K, S109N, S109R, S110P, Q111R, I112T, K113N, K113E, K114N, K114M, K114E, G116D, K118N, K118R, T119I, D120V, K123N, L129M, I130V, K131R, A132S, K134M, K134N, F142V, L145M, I146T, E147K, F148S, S150F, R154K, Q155H, E166D, K169E, P178S, A180V, A181T, A181S, I183V, A184S, A184T, A184V, P187S, A190T, A190V, V194M, V194A, R197I, Y201N, L204M, D207N, K209N, Q213H, Q213V, A219S, K221N, D225N, V226E, P227T, K229E, S232N, K233N, K233R, N234H, T236A, A238V, A238S, A241S, E246D, K251N, H252Y, H252R, E256D, A257S, A261V, S263I, S263N, N265D, Y267C, E269K, E269D, K271E, K271R, H272Y, I274V, F280L, D281N, D281G, K285G, K286N, K288R, S291F, S291P, K292N, K296R, K296N, I299S, D301G, E303D, I304T, I304V, E306G, V307L, V307G, V307A, V307D, V307G, I308N, N310S, Y313H, N314K, N316K, N316D, A317D, L318Q, D319N, P320S, P320L, M323I, L324M, D326N, V328M, V328A, A330D, I331V, V332G, S340L, T341A, A343G, S344N, I355V, F412V, V418F, Y427C, R514K, S1198L, A1201V, G1206S, C1212G, F1260L, and V1282M, relative to SEQ ID NO: 6. In some embodiments, the TnsC protein does not include one or more substitutions selected from: E6D, 19F, I28T, D44N, D44G, H64Y, S76Y, M80I, A95T, S110P, K113N, K113E, K114N, K114E, G116D, K118N, K118R, I130V, A132S, F142V, Q155H, P187S, A190T, V194A, K221N, K233R, N234H, A238V, A261V, H272Y, F280L, D281G, I299D, E303F, I304T, V307S, I308N, Y313H, N316D, and V328M, relative to SEQ ID NO: 6.

In some embodiments, the TnsC protein comprises an amino acid sequence having amino acid substitutions at positions: 2, 67, 95, and 226; 6 and 316; 38, 95, 303; 44 and 118; 67, 95, and 226; 44 and 76; 44, 76, and 118; 130, 234, 303; 118 and 1201; 118, 1201, and 44; 118, 1201, and 76; 130, 234, and 303; 154 and 269; 221 and 44; 44, 76, 130, 234, and 303; 44, 76, 118, and 1201; 197 and 314; 76, 181, and 194; 76, 118, 252, and 292; 76 and 274; 76, 102, 118, and 307; 12 and 76; 67, 95, and 226; 26 and 76; 22, 76, 319; 154 and 269; 76 and 238; 76, 238, 296, and 328; 7 and 76; 76 and 263; 59, 76, 306, and 316; or 280 and 340, relative to SEQ ID NO: 6. In select embodiments, the TnsC protein comprises an amino acid sequence having an amino acid substitution at position 76, relative to SEQ ID NO: 6.

In some embodiments, the TnsC protein comprises an amino acid sequence having amino acid substitutions of: N2S, K67N or K67R, A95D, and V226E, relative to SEQ ID NO: 6. In some embodiments, the TnsC protein comprises an amino acid sequence having amino acid substitutions of: E6D and N316K or N316D, relative to SEQ ID NO: 6. In some embodiments, the TnsC protein comprises an amino acid sequence having amino acid substitutions of: N38S, A95D, E303D, relative to SEQ ID NO: 6. In some embodiments, the TnsC protein comprises an amino acid sequence having amino acid substitutions of: K67N or K67R, A95D, and V226E, relative to SEQ ID NO: 6. In some embodiments, the TnsC protein comprises an amino acid sequence having amino acid substitutions of: D44G or D44N and S76Y or K118N or K118R, relative to SEQ ID NO: 6. In some embodiments, the TnsC protein comprises an amino acid sequence having amino acid substitutions of: D44G or D44N, 1130V, N234H, E303D, relative to SEQ ID NO: 6. In some embodiments, the TnsC protein comprises an amino acid sequence having amino acid substitutions of: K118N or K118R and A1201V, relative to SEQ ID NO: 6. In some embodiments, the TnsC protein further comprises amino acid substitutions of: D44G or D44N or S76Y. In some embodiments, the TnsC protein comprises an amino acid sequence having amino acid substitutions of: I130V, N234H, and E303D, relative to SEQ ID NO: 6. In some embodiments, the TnsC protein comprises an amino acid sequence having amino acid substitutions of: R154K and E269K or E269D, relative to SEQ ID NO: 6. In some embodiments, the TnsC protein comprises an amino acid sequence having amino acid substitutions of: K221N and D44G or D44N, relative to SEQ ID NO: 6. In some embodiments, the TnsC protein comprises an amino acid sequence having amino acid substitutions of: F280L and S340L, relative to SEQ ID NO: 6. In some embodiments, the TnsC protein comprises an amino acid sequence having amino acid substitutions of: D44N or D44G, S76Y, 1130V, N234H, and E303D, relative to SEQ ID NO: 6. In some embodiments, the TnsC protein comprises an amino acid sequence having amino acid substitutions of: D44N or D44G, S76Y, K118R, and A1201V, relative to SEQ ID NO: 6. In select embodiments, the TnsC protein comprises an amino acid sequence having an amino acid substitutions of: S76Y, relative to SEQ ID NO: 6. In select embodiments, the TnsC protein comprises an amino acid sequence having an amino acid substitutions of: R197I and N314K, relative to SEQ ID NO: 6. In select embodiments, the TnsC protein comprises an amino acid sequence having an amino acid substitutions of: S76Y, A181S, and V194M, relative to SEQ ID NO: 6. In select embodiments, the TnsC protein comprises an amino acid sequence having an amino acid substitutions of: S76Y, K118R, H252R, and K292N, relative to SEQ ID NO: 6. In select embodiments, the TnsC protein comprises an amino acid sequence having an amino acid substitutions of: S76Y and I274V, relative to SEQ ID NO: 6. In select embodiments, the TnsC protein comprises an amino acid sequence having an amino acid substitutions of: S76Y, A102T, K118R, and V307G, relative to SEQ ID NO: 6. In select embodiments, the TnsC protein comprises an amino acid sequence having an amino acid substitutions of: L12M and S76Y, relative to SEQ ID NO: 6. In select embodiments, the TnsC protein comprises an amino acid sequence having an amino acid substitutions of: K67N, A95D, and V226E, relative to SEQ ID NO: 6. In select embodiments, the TnsC protein comprises an amino acid sequence having an amino acid substitutions of: K26N and S76Y, relative to SEQ ID NO: 6. In select embodiments, the TnsC protein comprises an amino acid sequence having an amino acid substitutions of: H22Y, S76Y, and D319N, relative to SEQ ID NO: 6. In select embodiments, the TnsC protein comprises an amino acid sequence having an amino acid substitutions of: R154K and E269D, relative to SEQ ID NO: 6. In select embodiments, the TnsC protein comprises an amino acid sequence having an amino acid substitutions of: S76Y and A238S, relative to SEQ ID NO: 6. In select embodiments, the TnsC protein comprises an amino acid sequence having an amino acid substitutions of: S76Y and S263N, relative to SEQ ID NO: 6. In select embodiments, the TnsC protein comprises an amino acid sequence having an amino acid substitutions of: S59T, S76Y, E306G, and N316D, relative to SEQ ID NO: 6.

In select embodiments, the TnsC protein comprises an amino acid sequence having an amino acid substitutions of: S76Y, A238S, K296N, and V328M, relative to SEQ ID NO: 6. In select embodiments, the TnsC protein comprises an amino acid sequence having an amino acid substitutions of: S76Y and S263N, relative to SEQ ID NO: 6. In select embodiments, the TnsC protein comprises an amino acid sequence having an amino acid substitutions of: L12M and S76Y, relative to SEQ ID NO: 6. In select embodiments, the TnsC protein comprises an amino acid sequence having an amino acid substitutions of: I7V and S76Y, relative to SEQ ID NO: 6.

In some embodiments, the systems comprise one or more of TnsA, TnsB, and TnsC proteins having one or more amino acid substitutions, deletions, or additions relative to SEQ ID NOs: 4-6, as shown in Table 2.

In some embodiments at least one of the one or more Cas proteins and the one or more transposon-associated proteins are provided as a fusion protein. For example, at least one of the one or more Cas proteins and the one or more transposon-associated proteins may be in a fusion protein with a wild-type version of a Cas protein or transposon-associated protein. Alternatively, at least two of the disclosed modified Cas proteins or transposon-associated proteins may be linked in a fusion protein. In some embodiments, each of the one or more Cas proteins and the one or more transposon-associated proteins are provided as a single fusion protein.

In some embodiments, TnsA and TnsB are provided as a TnsA-TnsB fusion protein. TnsA and TnsB can be fused in any orientation: N-terminus to C-terminus; C-terminus to N-terminus; N-terminus to N-terminus; or C-terminus to C-terminus, respectively. Preferably the C-terminus of TnsA is fused to the N-terminus of TnsB.

In some embodiments, any of the fusion proteins (e.g., the TnsA-TnsB fusion) may be fused using an amino acid linker peptide of various lengths to provide greater physical separation and allow more spatial mobility between the fused portions. The linker may comprise any amino acids and may be of any length. In some embodiments, the linker may be less than about 50 (e.g., 40, 30, 20, 10, or 5) amino acid residues.

In some embodiments, the linker is a flexible linker, such that the individual proteins (e.g., TnsA and TnsB) can have orientation freedom in relationship to each other. For example, a flexible linker may include amino acids having relatively small side chains, and which may be hydrophilic. Without limitation, the flexible linker may contain a stretch of glycine and/or serine residues. In some embodiments, the linker comprises at least one glycine-rich region. For example, the glycine-rich region may comprise a sequence comprising [GS]n, wherein n is an integer between 1 and 10.

In some embodiments, the linker further comprises a nuclear localization sequence (NLS). The NLS may be embedded within a linker sequence, such that it is flanked by additional amino acids. In some embodiments, the NLS is flanked on each end by at least a portion of a flexible linker. In some embodiments, the NLS is flanked on each end by a glycine rich region of the linker. Suitable nuclear localization sequences for use with the disclosed system are described further below and are applicable to use with the fusion proteins herein, e.g., TnsA-TnsB fusion protein.

In the systems disclosed herein, at least one of the one or more Cas protein and the one or more transposon-associated protein comprise at least one nuclear localization sequence (NLS). The at least one nuclear localization sequence may be appended to at least one of the one or more Cas protein and the one or more transposon-associated protein at a N-terminus, a C-terminus, embedded in the protein (e.g., inserted internally within the open reading frame (ORF)), or a combination thereof.

The nuclear localization sequence may comprise any amino acid sequence known in the art to functionally tag or direct a protein for import into a cell's nucleus (e.g., for nuclear transport). Usually, a nuclear localization sequence comprises one or more positively charged amino acids, such as lysine and arginine.

In some embodiments, the NLS is a monopartite sequence. A monopartite NLS comprise a single cluster of positively charged or basic amino acids. In some embodiments, the monopartite NLS comprises a sequence of K-K/R-X-K/R, wherein X can be any amino acid. Exemplary monopartite NLSs include those from the SV40 large T-antigen, c-Myc, and TUS-proteins, as described elsewhere herein.

In some embodiments, the NLS is a bipartite sequence. Bipartite NLSs comprise two clusters of basic amino acids, separated by a spacer of about 9-12 amino acids. Exemplary bipartite NLSs include the NLS of nucleoplasmin, KR[PAATKKAGQA]KKKK (SEQ ID NO: 17) and the NLS of EGL-13, MSRRRKANPTKLSENAKKLAKEVEN (SEQ ID NO: 15). In some embodiments, the NLS comprises a bipartite SV40 NLS. In certain embodiments, the NLS comprises an amino acid sequence having at least 70% (e.g., having at least 75%, at least 80%, at least 85%, at least 90%, at least 92%, at least 95%, at least 96%, at least 97%, at least 98%, or at least 99%) similarity to KRTADGSEFESPKKKRKV (SEQ ID NO: 19). In select embodiments, the NLS consists of an amino acid sequence of KRTADGSEFESPKKKRKV (SEQ ID NO: 19).

The protein components of the disclosed system (e.g., the Cas proteins or the transposon-associated proteins) may further comprise an epitope tag (e.g., 3×FLAG tag, an HA tag, a Myc tag, and the like). In some embodiments, the epitope tag may be adjacent, either upstream or downstream, to a nuclear localization sequence. The epitope tags may be at the N-terminus, a C-terminus, or a combination thereof of the corresponding protein.

In some embodiments, the systems may further comprise a guide RNA (gRNA) or a nucleic acid encoding a gRNA, wherein the gRNA is complementary to at least a portion of a target nucleic acid sequence. In some embodiments, one or more of the at least one Cas protein are part of a ribonucleoprotein (RNP) complex with the gRNA.

The gRNA may be a crRNA, crRNA/tracrRNA (or single guide RNA, sgRNA). The terms “gRNA,” “guide RNA,” “crRNA,” and “CRISPR guide sequence” may be used interchangeably throughout and refer to a nucleic acid comprising a sequence that determines the binding specificity of the CRISPR-Cas system. A gRNA hybridizes to (complementary to, partially or completely) a target nucleic acid sequence (e.g., the genome in a host cell). In some embodiments, the at least one gRNA is encoded in a CRISPR RNA (crRNA) array.

The gRNA or portion thereof that hybridizes to the target nucleic acid (a target site) may be any length. In some embodiments, the gRNA sequence that hybridizes to the target nucleic acid is 15, 16, 17, 18, 19, 20, 21, 22, 23, 24, 25, 26, 27, 28, 29, 30, 31, 32, 33, 34, 35, 36, 37, 38, 39, or 40 nucleotides in length. gRNAs or sgRNA(s) used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer).

To facilitate gRNA design, many computational tools have been developed (See Prykhozhij et al. (PLOS ONE, 10 (3): (2015)); Zhu et al. (PLOS ONE, 9 (9) (2014)); Xiao et al. (Bioinformatics. January 21 (2014)); Heigwer et al. (Nat Methods, 11 (2): 122-123 (2014)). Methods and tools for guide RNA design are discussed by Zhu (Frontiers in Biology, 10 (4) pp 289-296 (2015)), which is incorporated by reference herein. Additionally, there are many publicly available software tools that can be used to facilitate the design of sgRNA(s); including but not limited to, Genscript Interactive CRISPR gRNA Design Tool, WU-CRISPR, and Broad Institute GPP sgRNA Designer. There are also publicly available pre-designed gRNA sequences to target many genes and locations within the genomes of many species (human, mouse, rat, zebrafish, C. elegans), including but not limited to, IDT DNA Predesigned Alt-R CRISPR-Cas9 guide RNAs, Addgene Validated gRNA Target Sequences, and GenScript Genome-wide gRNA databases.

In addition to a sequence that binds to a target nucleic acid, in some embodiments, the gRNA may also comprise a scaffold sequence (e.g., tracrRNA). In some embodiments, such a chimeric gRNA may be referred to as a single guide RNA (sgRNA). Exemplary scaffold sequences will be evident to one of skill in the art and can be found, for example, in Jinek, et al. Science (2012) 337 (6096): 816-821, and Ran, et al. Nature Protocols (2013) 8:2281-2308, incorporated herein by reference in their entireties.

In some embodiments, the gRNA sequence does not comprise a scaffold sequence and a scaffold sequence is expressed as a separate transcript. In such embodiments, the gRNA sequence further comprises an additional sequence that is complementary to a portion of the scaffold sequence and functions to bind (hybridize) the scaffold sequence.

The gRNA can comprise spacer sequence. The space sequence can be any length. In some embodiments, the space sequence is 30-40 nucleotides long (e.g., 30, 31, 32, 33, 34, 35, 36, 37, 38, 39, or 40).

In some embodiments, the gRNA sequence is at least 50%, 55%, 60%, 65%, 70%, 75%, 80%, 85%, 90%, 95%, 96%, 97%, 98%, 99%, or at least 100% complementary to a target nucleic acid. In some embodiments, the gRNA sequence is at least 50%, 55%, 60%, 65%, 70%, 75%, 80%, 85%, 90%, 95%, 96%, 97%, 98%, 99%, or at least 100% complementary to the 3′ end of the target nucleic acid (e.g., the last 5, 6, 7, 8, 9, or 10 nucleotides of the 3′ end of the target nucleic acid).

The gRNA may be a non-naturally occurring gRNA.

The system may further comprise a target nucleic acid. The terms “target sequence,” “target nucleic acid,” and “target site” (e.g., a “target genomic DNA sequence”) are used interchangeably herein to refer to a polynucleotide (nucleic acid, gene, chromosome, genome, etc.) to which a guide sequence (e.g., a synthetic guide RNA) is designed to have complementarity, wherein hybridization between the target sequence and a guide sequence promotes the formation of a CRISPR complex, provided sufficient conditions for binding exist. The target sequence and guide sequence need not exhibit complete complementarity, provided that there is sufficient complementarity to cause hybridization and promote formation of a CRISPR complex. A target sequence may comprise any polynucleotide, such as DNA or RNA. Suitable DNA/RNA binding conditions include physiological conditions normally present in a cell. Other suitable DNA/RNA binding conditions (e.g., conditions in a cell-free system) are known in the art.

The target sequence may or may not be flanked by a protospacer adjacent motif (PAM) sequence. In certain embodiments, a nucleic acid-guided nuclease can only cleave a target sequence if an appropriate PAM is present, see, for example Doudna et al., Science, 2014, 346 (6213): 1258096, incorporated herein by reference. A PAM can be 5′ or 3′ of a target sequence. A PAM can be upstream or downstream of a target sequence. In one embodiment, the target sequence is immediately flanked on the 3′ end by a PAM sequence. A PAM can be 1, 2, 3, 4, 5, 6, 7, 8, 9, 10 or more nucleotides in length. In certain embodiments, a PAM is between 2-6 nucleotides in length. The target sequence may or may not be located adjacent to a PAM sequence (e.g., PAM sequence located immediately 3′ of the target sequence) (e.g., for Type I CRISPR/Cas systems). In some embodiments, e.g., Type I systems, the PAM is on the alternate side of the protospacer (the 5′ end). Makarova et al. describes the nomenclature for all the classes, types, and subtypes of CRISPR systems (Nature Reviews Microbiology 13:722-736 (2015)). Guide structures and PAMs are described in by R. Barrangou (Genome Biol. 16:247 (2015)).

Non-limiting examples of the PAM sequences include: CC, CA, AG, GT, TA, AC, CA, GC, CG, GG, CT, TG, GA, AGG, TGG, T-rich PAMs (such as TTT, TTG, TTC, etc.), NGG, NGA, NAG, NGGNG and NNAGAAW (W=A or T), NNNNGATT, NAAR (R=A or G), NNGRR (R=A or G), NNAGAA, and NAAAAC, where N is any nucleotide. In some embodiments, the PAM may comprise a sequence of CN, in which N is any nucleotide. In select embodiments, the PAM may comprise a sequence of CC.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization. There may be mismatches distal from the PAM.

In some embodiments, when the system comprises TnsA, TnsB, TnsC, TnsD and TniQ, binding to the target nucleic acid may be mediated through a TnsD binding site within the target nucleic acid sequence. Thus, the recognition of the target nucleic acid utilizing the systems described herein may proceed in a gRNA-dependent and/or-independent manner.

The system may further include a donor nucleic acid. The donor nucleic acid may be a part of a bacterial plasmid, bacteriophage, a virus, autonomously replicating extra chromosomal DNA element, linear plasmid, linear DNA, linear covalently closed DNA, mitochondrial or other organellar DNA, chromosomal DNA, and the like. In some embodiments, the donor nucleic acid comprises a cargo nucleic acid sequence.

The donor nucleic acid may be flanked by at least one transposon end sequence. In some embodiments, the donor nucleic acid is flanked on the 5′ and the 3′ end with a transposon end sequence. The term “transposon end sequence” refers to any nucleic acid comprising a sequence capable of forming a complex with the transposase enzymes thus designating the nucleic acid between the two ends for rearrangement. Usually, these sequences contain inverted repeats and may be about 10-150 base pairs long, however the exact sequence requirements differ for the specific transposase enzymes. Transposon end sequences are well known in the art. Transposon ends sequences may or may not include additional sequences that promotes or augment transposition.

The transposon end sequences on either end may be the same or different. The transposon end sequence may be the endogenous CRISPR-transposon end sequences or may include deletions, substitutions, or insertions. The endogenous CRISPR-transposon end sequences may be truncated. In some embodiments, the transposon end sequence includes an about 40 base pair (bp) deletion relative to the endogenous CRISPR-transposon end sequence. In some embodiments, the transposon end sequence includes an about 100 base pair deletion relative to the endogenous CRISPR-transposon end sequence. The deletion may be in the form of a truncation at the distal (in relation to the cargo) end of the transposon end sequences.

The donor nucleic acid, and by extension the cargo nucleic acid, may of any suitable length, including, for example, about 50-100 bp (base pairs), about 100-1000 bp, at least or about 10 bp, at least or about 20 bp, at least or about 25 bp, at least or about 30 bp, at least or about 35 bp, at least or about 40 bp, at least or about 45 bp, at least or about 50 bp, at least or about 55 bp, at least or about 60 bp, at least or about 65 bp, at least or about 70 bp, at least or about 75 bp, at least or about 80 bp, at least or about 85 bp, at least or about 90 bp, at least or about 95 bp, at least or about 100 bp, at least or about 200 bp, at least or about 300 bp, at least or about 400 bp, at least or about 500 bp, at least or about 600 bp, at least or about 700 bp, at least or about 800 bp, at least or about 900 bp, at least or about 1 kb (kilobase pair), at least or about 2 kb, at least or about 3 kb, at least or about 4 kb, at least or about 5 kb, at least or about 6 kb, at least or about 7 kb, at least or about 8 kb, at least or about 9 kb, at least or about 10 kb, or greater. In some embodiments, the system comprises components from or derived from different CRISPR-Tn systems. In some embodiments, at least one of the one or more Cas proteins and the one or more transposon-associated proteins may be derived from a homologous CRISPR-transposon system compared to the other protein components in the system.

In some embodiments, the system comprises two or more engineered CRISPR-Tn systems. Pairing of orthogonal systems with their orthogonal donor DNA substrates enables tandem insertion of multiple distinct payloads directly adjacent to each other without any risk of repressive effects from target immunity. For example, one, two, three, four, five, or more orthogonal CRISPR-Tn systems may be used to integrate large tandem arrays of payload DNA. In some embodiments, multiple orthogonal RNA-guided transposases and their transposon donor DNAs may be integrated into distal regions of a given chromosome or genome, such that the lack of sequence identity between the transposon ends of the distinct transposon DNA substrates prevents genetic instability and the risk of recombination.

Sequences of exemplary Cas proteins, transposon-associated proteins, gRNAs, and transposon ends can also be found in International Patent Publication WO 2020/181264 and International Patent Application PCT/US2022/032541, incorporated herein by reference. However, the invention is not limited to the disclosed or referenced exemplary sequences. Indeed, genetic sequences can vary between different strains, and this natural scope of allelic variation is included within the scope of the invention.

The system may be a cell free system. Also disclosed is a cell comprising the system described herein. In some embodiments, the cell is a prokaryotic cell. In some embodiments, the cell is a eukaryotic cell. In some embodiments, the cell is a mammalian cell (e.g., a cell of a non-human primate or a human cell). Thus, in some embodiments, disclosed herein are systems or kits for DNA integration into a target nucleic acid sequence in a eukaryotic cell (e.g., a mammalian cell, a human cell).

The one or more nucleic acids encoding the engineered CRISPR-Tn system may be any nucleic acid including DNA, RNA, or combinations thereof. In some embodiments, the one or more nucleic acids comprise one or more messenger RNAs, one or more vectors, or any combination thereof.

The one or more Cas proteins, the one or more transposon-associated protein (e.g., TnsA, TnsB, TnsC, TnsD, and TniQ), the at least one gRNA, and the donor nucleic acid may be on the same or different nucleic acids (e.g., vector(s)). In some embodiments, the one or more Cas proteins are encoded by a single nucleic acid. In some embodiments, the one or more transposon-associated proteins are encoded by a single nucleic acid. In some embodiments, the nucleic acid encoding the one or more Cas proteins also encodes the one or more transposon-associated proteins. In some embodiments, the one or more Cas proteins are encoded by a different nucleic acid from the one or more transposon-associated proteins.

In some embodiments, the at least one gRNA is encoded by a nucleic acid different from the nucleic acid(s) encoding the one or more Cas proteins and the one or more transposon-associated proteins. In some embodiments, the at least one gRNA is encoded by a nucleic acid also encoding at least one Cas protein, at least one transposon-associated protein, or both. In some embodiments, the one or more Cas proteins, the one or more transposon-associated proteins, and the at least one gRNA are encoded by a single nucleic acid. The gRNA may be encoded anywhere in the nucleic acid encoding the one or more Cas proteins or the one or more transposon-associated proteins. In some embodiments, the gRNA is encoded in the 3′ UTR of a protein coding nucleic acid.

In some embodiments, the nucleic acid encoding the one or more Cas proteins, the one or more transposon-associated protein, the at least one gRNA, or any combination thereof further comprises the donor nucleic acid.

The present systems may further include at least one unfoldase protein. Unfoldases are proteins that catalyze the unfolding of a native protein without affecting the primary structure. The unfoldase may be an NTP driven unfoldase. NTP driven unfoldases may include ATP-dependent proteases, including, but not limited to, ATPases, AAA proteases, or AAA+ enzymes (e.g., AAA+ enzyme). In some embodiments, the at least one unfoldase protein may comprise ClpX (caseinolytic mitochondrial matrix peptidase chaperone subunit X). In some embodiments, the at least one unfoldase protein may comprise a homolog of ClpX.

ClpX homologs may be readily screened through systematic testing and optimization of a large panel of homologs, identified through bioinformatic search strategies such as BLASTp and psi-BLASTp. In some embodiments, the unfoldase protein (e.g., ClpX) is derived from the same host organism as that of the engineered CAST system. In some embodiments, the unfoldase protein (e.g., ClpX) is derived from a different host organism as that of the engineered CAST system. As such, the at least one unfoldase protein (e.g., ClpX) is not limited from which organism it is derived. In some embodiments, the unfoldase protein (e.g., ClpX) is derived from the E. coli genome. In other embodiments, the unfoldase protein (e.g., ClpX) from the cognate strain from which the engineered CAST system is derived. For example, the unfoldase protein from Vibrio cholerae HE-45 can be used alongside RNA-guided DNA integration machinery derived from Tn6677, while unfoldase proteins from Pseudoalteromonas sp. S983 can be used alongside RNA-guided DNA integration machinery derived from Tn7016.

In some embodiments, the systems further comprise one or more additional genome engineering tools. For example, the systems may further comprise nucleases, such as zinc finger nucleases (ZFNs) and/or transcription activator like effector nucleases (TALENs); transcriptional activators, transcriptional repressors, histone-modifying proteins, integrases, and recombinases.

### Nucleic Acids and Delivery

The present disclosure also provides for nucleic acids encoding the polypeptides, compositions comprising nucleic acids encoding the polypeptide and systems comprising nucleic acids encoding the polypeptides disclosed herein, and vectors containing or encoding these nucleic acids. The vectors may be used to propagate the nucleic acid in an appropriate cell and/or to allow expression from the nucleic acid (e.g., an expression vector). The person of ordinary skill in the art would be aware of the various vectors available for propagation and expression of a nucleic acid sequence.

The present disclosure further provides engineered, non-naturally occurring vectors and vector systems, which can encode one or more of the peptides or components of the present systems. The vector(s) can be introduced into a cell that is capable of expressing the polypeptide encoded thereby, including any suitable prokaryotic or eukaryotic cell.

The vectors of the present disclosure may be delivered to a eukaryotic cell in a subject. Modification of the eukaryotic cells via the present system can take place in a cell culture, where the method comprises isolating the eukaryotic cell from a subject prior to the modification. In some embodiments, the method further comprises returning said eukaryotic cell and/or cells derived therefrom to the subject.

Viral and non-viral based gene transfer methods can be used to introduce nucleic acids encoding the disclosed polypeptides or components of the present system into cells, tissues, or a subject. Such methods can be used to administer nucleic acids encoding the disclosed polypeptides or components of the present system to cells in culture, or in a host organism. Non-viral vector delivery systems include DNA plasmids, cosmids, RNA (e.g., a transcript of a vector described herein), a nucleic acid, and a nucleic acid complexed with a delivery vehicle. Viral vector delivery systems include DNA and RNA viruses, which have either episomal or integrated genomes after delivery to the cell. Viral vectors include, for example, retroviral, lentiviral, adenoviral, adeno-associated and herpes simplex viral vectors.

In certain embodiments, plasmids that are non-replicative, or plasmids that can be cured by high temperature may be used, such that any or all of the necessary components of the system may be removed from the cells under certain conditions. For example, this may allow for DNA integration by transforming bacteria of interest, but then being left with engineered strains that have no memory of the plasmids or vectors used for the integration. Drug selection strategies may be adopted for positively selecting for cells. A donor nucleic acid may contain one or more drug-selectable markers within the cargo. Then presuming that the original donor plasmid is removed, drug selection may be used to enrich for integrated clones. Colony screenings may be used to isolate clonal events.

A variety of viral constructs may be used to deliver the disclosed polypeptides or components of the present system (such as one or more Cas proteins and/or Tns proteins, gRNA(s), donor DNA, etc.) to the targeted cells and/or a subject. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7 (1): 33-40; and Walther W. and Stein U., 2000 Drugs, 60 (2): 249-71, incorporated herein by reference.

In one embodiment, a nucleic acid encoding the disclosed polypeptides or components of the present system is contained in a plasmid vector that allows expression of the disclosed polypeptides or components of the present system and subsequent isolation and purification of from the recombinant vector. Accordingly, the disclosed polypeptides or components of the present system disclosed herein can be purified following expression, obtained by chemical synthesis, or obtained by recombinant methods.

To construct cells that express the disclosed polypeptides or components of the present system, expression vectors for stable or transient expression of the disclosed polypeptides or components of the present system may be constructed via conventional methods as described herein and introduced into host cells. For example, nucleic acids encoding the components of the disclosed polypeptides or components of the present system may be cloned into a suitable expression vector, such as a plasmid or a viral vector in operable linkage to a suitable promoter. The selection of expression vectors/plasmids/viral vectors should be suitable for integration and replication in eukaryotic cells.

In certain embodiments, vectors of the present disclosure can drive the expression of one or more sequences in prokaryotic cells. Promoters that may be used include T7 RNA polymerase promoters, constitutive E. coli promoters, and promoters that could be broadly recognized by transcriptional machinery in a wide range of bacterial organisms. The system may be used with various bacterial hosts.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible and tissue specific expression of a RNA, transmembrane proteins, or other proteins can be accomplished by placing the nucleic acid encoding such a molecule under the control of an inducible or tissue specific promoter/regulatory sequence. Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. Various commercially available ubiquitous as well as tissue-specific promoters and tumor-specific are available, for example from InvivoGen. In addition, promoters which are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

The vectors of the present disclosure may direct expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′- and 3′-untranslated regions for mRNA stability and translation efficiency from highly-expressed genes like α-globin or β-globin; SV40 polyoma origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric receptor. Suitable vectors and methods for producing vectors containing transgenes are well known and available in the art. Selectable markers also include chloramphenicol resistance, tetracycline resistance, spectinomycin resistance, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

When introduced into the cell, the vectors may be maintained as an autonomously replicating sequence or extrachromosomal element or may be integrated into host DNA.

In one embodiment, the donor DNA may be delivered using the same gene transfer system as used to deliver the Cas protein, and/or transposon-associated proteins (included on the same vector) or may be delivered using a different delivery system. In another embodiment, the donor DNA may be delivered using the same transfer system as used to deliver gRNA(s).

In one embodiment, the present disclosure comprises integration of exogenous DNA into the endogenous gene. Alternatively, an exogenous DNA is not integrated into the endogenous gene. The DNA may be packaged into an extrachromosomal or episomal vector (such as AAV vector), which persists in the nucleus in an extrachromosomal state, and offers donor-template delivery and expression without integration into the host genome. Use of extrachromosomal gene vector technologies has been discussed in detail by Wade-Martins R (Methods Mol Biol. 2011; 738:1-17, incorporated herein by reference).

The disclosed polypeptides or components of the present system (e.g., proteins, polynucleotides encoding these proteins, donor polynucleotides and compositions comprising the proteins and/or polynucleotides described herein) may be delivered by any suitable means. In certain embodiments, the polypeptides or system is delivered in vivo. In other embodiments, the polypeptides or system is delivered to isolated/cultured cells (e.g., autologous iPS cells) in vitro to provide modified cells useful for in vivo delivery to patients afflicted with a disease or condition.

Vectors according to the present disclosure can be transformed, transfected, or otherwise introduced into a wide variety of cells. Transfection refers to the taking up of a vector by a cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Any of the vectors comprising a nucleic acid sequence that encodes the disclosed polypeptides or components of the present system is also within the scope of the present disclosure. Such a vector may be delivered into host cells by a suitable method. Methods of delivering vectors to cells are well known in the art and may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110 (6): 2082-2087, incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more transgenes can be delivered by any method appropriate for introducing nucleic acids into a cell. In some embodiments, the construct or the nucleic acid encoding the disclosed polypeptides or components of the present system is a DNA molecule. In some embodiments, the nucleic acid encoding the disclosed polypeptides or components of the present system is a DNA vector and may be electroporated to cells. In some embodiments, the nucleic acid encoding the disclosed polypeptides or components of the present system is an RNA molecule, which may be electroporated to cells.

Additionally, delivery vehicles such as nanoparticle- and lipid-based mRNA or protein delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1:27) and Ibraheem et al. (Int J Pharm. 2014 Jan. 1; 459 (1-2): 70-83), incorporated herein by reference.

### Methods of Use

Also disclosed herein are methods for nucleic acid modification or integration utilizing the disclosed systems or compositions. The methods may comprise contacting a target nucleic acid sequence with a system, composition, or polypeptide disclosed herein. The descriptions and embodiments provided above for the systems, compositions, polypeptides, gRNA, and donor nucleic acid are applicable to the methods described herein.

The phrase “modifying a nucleic acid sequence” or “nucleic acid modification” as used herein, refers to modifying at least one physical feature of a nucleic acid sequence of interest. Nucleic acid modifications include, for example, single or double strand breaks, deletion, or insertion of one or more nucleotides, and other modifications that affect the structural integrity or nucleotide sequence of the nucleic acid sequence.

The target nucleic acid sequence may be in a cell. In some embodiments, contacting a target nucleic acid sequence comprises introducing the system, composition, or polypeptide into the cell. As described above the system, composition, or polypeptide may be introduced into eukaryotic or prokaryotic cells by methods known in the art. In some embodiments, the cell is a mammalian cell. In some embodiments, the cell is a human cell.

In some embodiments, the target nucleic acid is a nucleic acid endogenous to a target cell. In some embodiments, the target nucleic acid is a genomic DNA sequence. The term “genomic,” as used herein, refers to a nucleic acid sequence (e.g., a gene or locus) that is located on a chromosome in a cell.

In some embodiments, the target nucleic acid encodes a gene or gene product. The term “gene product,” as used herein, refers to any biochemical product resulting from expression of a gene. Gene products may be RNA or protein. RNA gene products include non-coding RNA, such as tRNA, rRNA, micro RNA (miRNA), and small interfering RNA (siRNA), and coding RNA, such as messenger RNA (mRNA). In some embodiments, the target nucleic acid sequence encodes a protein or polypeptide.

Polynucleotides containing the target nucleic acid sequence may include, but is not limited to, purified chromosomal DNA, total cDNA, cDNA fractionated according to tissue or expression state (e.g., after heat shock or after cytokine treatment other treatment) or expression time (after any such treatment) or developmental stage, plasmid, cosmid, BAC, YAC, phage library, etc. Polynucleotides containing the target site may include DNA from organisms such as Homo sapiens, Mus domesticus, Mus spretus, Canis domesticus, Bos, Caenorhabditis elegans, Plasmodium falciparum, Plasmodium vivax, Onchocerca volvulus, Brugia malayi, Dirofilaria immitis, Leishmania, Zea maize, Arabidopsis thaliana, Glycine max, Drosophila melanogaster, Saccharomyces cerevisiae, Schizosaccharomyces pombe, Neurospora, Escherichia coli, Salmonella typhimurium, Bacillus subtilis, Neisseria gonorrhoeae, Staphylococcus aureus, Streptococcus pneumonia, Mycobacterium tuberculosis, Aquifex, Thermus aquaticus, Pyrococcus furiosus, Thermus littoralis, Methanobacterium thermoautotrophicum, Sulfolobus caldoaceticus, and others.

The method may comprise administering to the subject, in vivo, or by transplantation of ex vivo treated cells, an effective amount of the described system, composition, or polypeptide. In some embodiments, the vector(s) is delivered to the tissue of interest by, for example, an intramuscular, intravenous, transdermal, intranasal, oral, mucosal, or other delivery methods.

The polypeptides, composition, components of the present system, or ex vivo treated cells may be administered with a pharmaceutically acceptable carrier or excipient as a pharmaceutical composition. In some embodiments, the polypeptides, composition, or components of the present system may be mixed, individually or in any combination, with a pharmaceutically acceptable carrier to form pharmaceutical compositions, which are also within the scope of the present disclosure.

In some embodiments, an effective amount of the polypeptides, components of the present system, or compositions as described herein can be administered. As used herein the term “effective amount” may be used interchangeably with the term “therapeutically effective amount” and refers to that quantity that is sufficient to result in a desired activity upon administration to a subject in need thereof. Within the context of the present disclosure, the term “effective amount” refers to that quantity of the components of the system such that successful DNA integration is achieved.

When utilized as a method of treatment, the effective amount may depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

In the context of the present disclosure insofar as it relates to any of the disease conditions recited herein, the terms “treat,” “treatment,” and the like mean to relieve or alleviate at least one symptom associated with such condition, or to slow or reverse the progression of such condition. Within the meaning of the present disclosure, the term “treat” also denotes to arrest, delay the onset (e.g., the period prior to clinical manifestation of a disease) and/or reduce the risk of developing or worsening a disease. For example, in connection with cancer the term “treat” may mean eliminate or reduce a patient's tumor burden, or prevent, delay, or inhibit metastasis, etc.

The phrase “pharmaceutically acceptable,” as used in connection with compositions and/or cells of the present disclosure, refers to molecular entities and other ingredients of such compositions that are physiologically tolerable and do not typically produce untoward reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g., Remington: The Science and Practice of Pharmacy 20th Ed. (2000) Lippincott Williams and Wilkins, Ed. K. E. Hoover.

The methods may be used for a variety of purposes. For example, the methods may include, but are not limited to, inactivation of a microbial gene, RNA-guided DNA integration in a plant or animal cell, methods of treating a subject suffering from a disease or disorder (e.g., cancer, Duchenne muscular dystrophy (DMD), sickle cell disease (SCD), β-thalassemia, and hereditary tyrosinemia type I (HT1)), and methods of treating a diseased cell (e.g., a cell deficient in a gene which causes cancer).

The disclosed methods may modify a target DNA sequence in a cell so as to modulate expression of the target DNA sequence, e.g., expression of the target DNA sequence is increased, decreased, or completely eliminated (e.g., via deletion of a gene). The modifications of the target sequence may lead to, for example, gene correction, gene replacement, gene tagging, transgene insertion, nucleotide deletion, gene disruption, gene mutation, gene knock-down, etc.

In some embodiments, the methods described herein may be used to correct one or more defects or mutations in a gene (referred to as “gene correction”). In such cases, the target sequence encodes a defective version of a gene, and the disclosed compositions and systems further comprise a donor nucleic acid molecule which encodes a wild-type or corrected version of the gene. Accordingly, in some embodiments, the methods described herein may be used to insert a gene or fragment thereof into a cell.

In another embodiment, the method of modifying a target sequence can be used to delete nucleic acids from a target sequence in a host cell by cleaving the target sequence and allowing the host cell to repair the cleaved sequence in the absence of an exogenously provided donor nucleic acid molecule. Deletion of a nucleic acid sequence in this manner can be used in a variety of applications, such as, for example, to remove disease-causing trinucleotide repeat sequences in neurons, to create gene knock-outs or knock-downs, and to generate mutations for disease models in research.

In some embodiments, the methods described herein may be used to genetically modify a plant or plant cell. As used herein, genetically modified plants include a plant into which has been introduced an exogenous polynucleotide. Genetically modified plants also include a plant that has been genetically manipulated such that endogenous nucleotides have been altered to include a mutation, such as a deletion, an insertion, a transition, a transversion, or a combination thereof. For instance, an endogenous coding region could be deleted. Such mutations may result in a polypeptide having a different amino acid sequence than was encoded by the endogenous polynucleotide. Another example of a genetically modified plant is one having an altered regulatory sequence, such as a promoter, to result in increased or decreased expression of an operably linked endogenous coding region. The genetically modified plant may promote a desired phenotypic or genotypic plant trait.

Genetically modified plants can potentially have improved crop yields, enhanced nutritional value, and increased shelf life. They can also be resistant to unfavorable environmental conditions, insects, and pesticides. The present systems and methods have broad applications in gene discovery and validation, mutational and cisgenic breeding, and hybrid breeding. The present methods may facilitate the production of a new generation of genetically modified crops with various improved agronomic traits such as herbicide resistance, herbicide tolerance, drought tolerance, male sterility, insect resistance, abiotic stress tolerance, modified fatty acid metabolism, modified carbohydrate metabolism, modified seed yield, modified oil percent, modified protein percent, resistance to bacterial disease, disease (e.g. bacterial, fungal, and viral) resistance, high yield, and superior quality. The present methods may also facilitate the production of a new generation of genetically modified crops with optimized fragrance, nutritional value, shelf-life, pigmentations (e.g., lycopene content), starch content (e.g., low-gluten wheat), toxin levels, propagation and/or breeding and growth time. See, for example, CRISPR/Cas Genome Editing and Precision Plant Breeding in Agriculture (Chen et al., Annu Rev Plant Biol. 2019 Apr. 29; 70:667-69), incorporated herein by reference.

The present method may confer one or more of the following traits to the plant cell: herbicide tolerance, drought tolerance, male sterility, insect resistance, abiotic stress tolerance, modified fatty acid metabolism, modified carbohydrate metabolism, modified seed yield, modified oil percent, modified protein percent, resistance to bacterial disease, resistance to fungal disease, and resistance to viral disease.

The present disclosure provides for a modified plant cell produced by the present method, a plant comprising the plant cell, and a seed, fruit, plant part, or propagation material of the plant. Transformed or genetically modified plant cells of the present disclosure may be as populations of cells, or as a tissue, seed, whole plant, stem, fruit, leaf, root, flower, stem, tuber, grain, animal feed, a field of plants, and the like. The present disclosure provides a transgenic plant. The transgenic plant may be homozygous or heterozygous for the genetic modification. Also provided by the present disclosure are transformed or genetically modified plant cells, tissues, plants, and products that contain the transformed or genetically modified plant cells. The present disclosure further encompasses the progeny, clones, cell lines or cells of the transgenic plants.

The present system and method may be used to modify a plant stem cell. The present disclosure further provides progeny of a genetically modified cell, where the progeny can comprise the same genetic modification as the genetically modified cell from which it was derived. The present disclosure further provides a composition comprising a genetically modified cell.

In one embodiment, the transformed or genetically modified cells, and tissues and products comprise a nucleic acid integrated into the genome, and production by plant cells of a gene product due to the transformation or genetic modification.

Methods of introducing exogenous nucleic acids into plant cells are well known in the art. Such plant cells are considered “transformed.” DNA constructs can be introduced into plant cells by various methods, including, but not limited to PEG- or electroporation-mediated protoplast transformation, tissue culture or plant tissue transformation by biolistic bombardment, or the Agrobacterium-mediated transient and stable transformation. The transformation can be transient or stable transformation. Suitable methods also include viral infection (such as double stranded DNA viruses), transfection, conjugation, protoplast fusion, electroporation, particle gun technology, calcium phosphate precipitation, direct microinjection, silicon carbide whiskers technology, Agrobacterium-mediated transformation, and the like. The choice of method is generally dependent on the type of cell being transformed and the circumstances under which the transformation is taking place (e.g., in vitro, ex vivo, or in vivo). Transformation methods based upon the soil bacterium Agrobacterium tumefaciens are useful for introducing an exogenous nucleic acid molecule into a vascular plant. The wild-type form of Agrobacterium contains a Ti (tumor-inducing) plasmid that directs production of tumorigenic crown gall growth on host plants. Transfer of the tumor-inducing T-DNA region of the Ti plasmid to a plant genome requires the Ti plasmid-encoded virulence genes as well as T-DNA borders, which are a set of direct DNA repeats that delineate the region to be transferred. An Agrobacterium-based vector is a modified form of a Ti plasmid, in which the tumor inducing functions are replaced by the nucleic acid sequence of interest to be introduced into the plant host.

Agrobacterium-mediated transformation generally employs cointegrate vectors or binary vector systems, in which the components of the Ti plasmid are divided between a helper vector, which resides permanently in the Agrobacterium host and carries the virulence genes, and a shuttle vector, which contains the gene of interest bounded by T-DNA sequences. A variety of binary vectors are well known in the art and are commercially available, for example, from Clontech (Palo Alto, Calif.). Methods of coculturing Agrobacterium with cultured plant cells or wounded tissue such as leaf tissue, root explants, hypocotyledons, stem pieces or tubers, for example, also are well known in the art. See., e.g., Glick and Thompson, (eds.), Methods in Plant Molecular Biology and Biotechnology, Boca Raton, Fla.: CRC Press (1993), incorporated herein by reference.

Microprojectile-mediated transformation also can be used to produce a transgenic plant. This method, first described by Klein et al. (Nature 327:70-73 (1987), incorporated herein by reference), relies on microprojectiles such as gold or tungsten that are coated with the desired nucleic acid molecule by precipitation with calcium chloride, spermidine, or polyethylene glycol. The microprojectile particles are accelerated at high speed into an angiosperm tissue using a device such as the BIOLISTIC PD-1000 (Biorad; Hercules Calif.).

In one embodiment, the present methods may be adapted to use in plants. The vectors may be optimized for transient expression of the present system in plant protoplasts, or for stable integration and expression in intact plants via the Agrobacterium-mediated transformation.

In certain embodiments, the present methods use a monocot promoter to drive the expression of one or more components of the present systems (e.g., gRNA) in a monocot plant. In certain embodiments, the present methods use a dicot promoter to drive the expression of one or more components of the present systems (e.g., gRNA) in a dicot plant.

The present methods may be used with various microbial species, including human pathogens that are medically important, and bacterial pests that are key targets within the agricultural industry, as well as antibiotic resistant versions thereof. The method may be designed to target any gene or any set of genes, such as virulence or metabolic genes, for clinical and industrial applications in other embodiments. For example, the present methods may be used to target and eliminate virulence genes from the population, to perform in situ gene knockouts, or to stably introduce new genetic elements to the metagenomic pool of a microbiome. The present systems and methods may be used to treat a multi-drug resistance bacterial infection in a subject. The present systems and methods may be used for genomic engineering within complex bacterial consortia.

The present systems and methods may be used to inactivate microbial genes. In some embodiments, the gene is an antibiotic resistance gene. For example, the coding sequence of bacterial resistance genes may be disrupted in vivo by insertion of a DNA sequence, leading to non-selective re-sensitization to drug treatment.

The methods described here also provide for treating a disease or condition in a subject. The method may comprise administering to the subject, in vivo, or by transplantation of ex vivo treated cells (e.g., disclosed T cells), a therapeutically effective amount of the present system, polypeptides, or components thereof.

In some embodiments, the methods are used to treat a pathogen or parasite on or in a subject by altering the pathogen or parasite. In some embodiments, the methods target a “disease-associated” gene. The term “disease-associated gene,” refers to any gene or polynucleotide whose gene products are expressed at an abnormal level or in an abnormal form in cells obtained from a disease-affected individual as compared with tissues or cells obtained from an individual not affected by the disease. A disease-associated gene may be expressed at an abnormally high level or at an abnormally low level, where the altered expression correlates with the occurrence and/or progression of the disease. A disease-associated gene also refers to a gene, the mutation or genetic variation of which is directly responsible or is in linkage disequilibrium with a gene(s) that is responsible for the etiology of a disease. Examples of genes responsible for such “single gene” or “monogenic” diseases include, but are not limited to, adenosine deaminase, α-1 antitrypsin, cystic fibrosis transmembrane conductance regulator (CFTR), β-hemoglobin (HBB), oculocutaneous albinism II (OCA2), Huntingtin (HTT), dystrophia myotonica-protein kinase (DMPK), low-density lipoprotein receptor (LDLR), apolipoprotein B (APOB), neurofibromin 1 (NF1), polycystic kidney disease 1 (PKD1), polycystic kidney disease 2 (PKD2), coagulation factor VIII (F8), dystrophin (DMD), phosphate-regulating endopeptidase homologue, X-linked (PHEX), methyl-CpG-binding protein 2 (MECP2), and ubiquitin-specific peptidase 9Y, Y-linked (USP9Y). Other single gene or monogenic diseases are known in the art and described in, e.g., Chial, H. Rare Genetic Disorders: Learning About Genetic Disease Through Gene Mapping, SNPs, and Microarray Data, Nature Education 1 (1): 192 (2008); Online Mendelian Inheritance in Man (OMIM); and the Human Gene Mutation Database (HGMD). In another embodiment, the target genomic DNA sequence can comprise a gene, the mutation of which contributes to a particular disease in combination with mutations in other genes. Diseases caused by the contribution of multiple genes which lack simple (i.e., Mendelian) inheritance patterns are referred to in the art as a “multifactorial” or “polygenic” disease. Examples of multifactorial or polygenic diseases include, but are not limited to, asthma, diabetes, epilepsy, hypertension, bipolar disorder, and schizophrenia. Certain developmental abnormalities also can be inherited in a multifactorial or polygenic pattern and include, for example, cleft lip/palate, congenital heart defects, and neural tube defects. In another embodiment, the target DNA sequence can comprise a cancer oncogene. The present disclosure provides for gene editing methods that can ablate a disease-associated gene (e.g., a cancer oncogene), which in turn can be used for in vivo gene therapy for patients. In some embodiments, the gene editing methods include donor nucleic acids comprising therapeutic genes.

### Kits

Also within the scope of the present disclosure are kits that include the polypeptides, compositions, or components of the present system.

The kit may include instructions for use in any of the methods described herein. The instructions can comprise a description of administration to a subject to achieve the intended effect. The instructions generally include information as to dosage, dosing schedule, and route of administration for the intended treatment. The kit may further comprise a description of selecting a subject suitable for treatment based on identifying whether the subject is in need of the treatment.

The kits provided herein are in suitable packaging. Suitable packaging includes, but is not limited to, vials, bottles, jars, flexible packaging, and the like. A kit may have a sterile access port (for example, the container may be an intravenous solution bag or a vial having a stopper pierceable by a hypodermic injection needle). The container may also have a sterile access port.

The packaging may be unit doses, bulk packages (e.g., multi-dose packages) or subunit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

The kit may further comprise a device for holding or administering the present system, polypeptides, or composition. The device may include an infusion device, an intravenous solution bag, a hypodermic needle, a vial, and/or a syringe.

The present disclosure also provides for kits for performing nucleic acid modification and integration in vitro. Optional components of the kit include one or more of the following: buffer constituents, control plasmid, sequencing primers, cells.

### EXAMPLES

The following are examples of the present invention and are not to be construed as limiting.

### Materials and Methods

General methods. Antibiotics (Gold Biotechnology) were used at the following working concentrations: carbenicillin 50 μg/mL, spectinomycin 50 μg/mL, chloramphenicol 25 μg/mL, kanamycin 50 μg/mL, tetracycline 10 μg/mL, streptomycin 50 μg/mL. Nuclease-free water (Qiagen) was used for PCRs and cloning. For all other experiments, water was purified using a MilliQ purification system (Millipore). Unless otherwise noted, Phusion U Hot Start or Phusion Hot Start II DNA polymerase (Thermo Fisher Scientific) were used for all PCRs. Unless otherwise noted, plasmids and selection phages (SPs) were cloned by USER assembly. Wild-type CAST gene sequences were obtained from the Sternberg lab. Plasmids were cloned and amplified using either Mach1 (Thermo Fisher Scientific) or Turbo (New England BioLabs) cells. Plasmid or SP DNA was amplified using the Illustra Templiphi 100 Amplification Kit (GE Healthcare Life Sciences) prior to Sanger sequencing. E. coli strain S2060 (Hubbard et al., Nat Methods 2015) was used in all phage propagations and plaque assays, and in all PACE experiments.

Phage propagation assay. Chemically competent S2060 E. coli cells were transformed with the circuit plasmids of interest as previously described (Wang et al., Nat Chem Biol 2018). Overnight cultures of single colonies grown in DRM media supplemented with maintenance antibiotics were diluted 1000-fold into DRM media with maintenance antibiotics and grown at 37° C. with shaking at 230 RPM to OD₆₀₀˜0.4-0.6. Cells were then infected with selection phage (SP) at an initial titer of 5×10⁵ pfu/mL. Cells were incubated for another 16-18 h at 37° C. with shaking at 230 RPM, then centrifuged at 4000 g for 2 min. The supernatant containing phage was removed and stored at 4° C. until use. Plasmid DNA from the pelleted host cells was isolated using a QIAprep spin miniprep kit (Qiagen) according to manufacturer instructions for subsequent measuring of integration at target sites.

Plaque assay. Overnight cultures of single E. coli cell colonies were grown in DRM media supplemented with maintenance antibiotics, then diluted 1000-fold into fresh DRM media with maintenance antibiotics and grown at 37° C. with shaking at 230 RPM to OD₆₀₀˜0.6-0.8 before use. SP were serially diluted 100-fold (4 dilutions total) in water. 10 μL of each phage dilution was combined with 150 μL of cells, and to this 1 mL of liquid (55° C.) top agar (2×YT media+0.5% agar) supplemented with 2% Bluo-gal (Gold Biotechnology) was added and mixed by pipetting up and down once. This mixture was then immediately pipetted onto one quadrant of a quartered Petri dish already containing 2 mL of solidified bottom agar (2×YT media+1.5% agar, no antibiotics). Plates were incubated at 37° C. for 16-18 h. Phage were plaqued on S2208 cells (S2060 cells transformed with pJC175e to enable activity-independent propagation), or on S2060 cells (to determine the presence of gIII-recombinant SP).

Phage-assisted non-continuous evolution. Phage-assisted non-continuous evolution (PANCE) was performed as previously reported (Miller et al., Nat Protoc 2020). Host and drift cells were freshly transformed for each experiment and kept for a week on agar plates at 4° C. For each passage, cells were grown to OD₆₀₀˜0.4 before adding SP and arabinose. Drifts were performed over the course of a day (˜6 h) and selections were performed overnight (˜12 h). SP titers were determined by plaque assay using S2208 cells.

Phage-assisted continuous evolution. Unless otherwise noted, PACE components, including host cell strains, lagoons, chemostats, and media, were all used as previously described (Miller et al., Nat Protoc 2020). Continuous dilution was performed using Masterflex L/S Digital Drive pumps (Cole-Parmer) fitted with Masterflex L/S Multichannel pump heads (Cole-Parmer).

Chemically competent S2060s were transformed with circuit plasmids and MP6, plated on 2×YT media+1.5% agar supplemented with 25 mM glucose (to prevent induction of mutagenesis) in addition to maintenance antibiotics, and grown at 37° C. for 18-20 h. Four colonies were picked into 1 mL DRM each in a 96-well deep well plate, and this was diluted 5-fold 8 times serially into DRM. The plate was sealed with a porous sealing film and grown at 37° C. with shaking at 230 RPM for 16-18 h. Dilutions with OD₆₀₀˜0.4-0.8 were then used to inoculate a chemostat containing 80 mL DRM. The chemostat was grown to OD₆₀₀˜0.4-0.6, then continuously diluted with fresh DRM at a rate of ˜1.5 chemostat volumes/h. The chemostat was maintained at a volume of 60-80 mL.

Prior to SP infection, lagoons were continuously diluted with culture from the chemostat at 1 lagoon vol/h and pre-induced with 10 mM arabinose for at least 2 h. Lagoons were infected with SP at a starting titer of 106 pfu/mL and maintained at a volume of 15 mL. Samples (500 μL) of the SP population were taken at indicated times from lagoon waste lines. These were centrifuged at 4000 g for 2 min, and the supernatant stored at 4° C. Lagoon titers were determined by plaque assays using S2208 cells. For Sanger sequencing of lagoons, single plaques were PCR amplified using primers AB1793 (5′-TAATGGAAACTTCCTCATGAAAAAGTCTTTAG; SEQ ID NO: 20) and AB1396 (5′-ACAGAGAGAATAACATAAAAACAGGGAAGC; SEQ ID NO: 21), both of which anneal to regions of the M13 phage backbone flanking the evolving gene of interest. Generally, 8 plaques were picked and sequenced per lagoon.

Evolution summary for Tn6677 TnsA, TnsB, and TnsC Throughout evolution of Tn6677 TnsA, TnsB, and TnsC, selection stringency was modulated by adjusting the amount of gIII expressed per integration event. This was done by tuning the strength of the ribosome binding site upstream gIII on the AP, and by adjusting the strength of the promoter in the transposon encoded by CP2. Tn6677 PANCE 1 on Tns circuit 2 was seeded with wild-type TnsA, TnsB, and TnsC and evolved for 15 passages under minimal selection stringency (SD8 RBS on AP, proC promoter on CP2). Due to gIII recombinant SP arising across all lagoons by passage 10, SP from PANCE 1 confirmed to lack gIII were isolated and used to seed Tn6677 PACE 1. Tn6677 PACE 1 was performed for 144 h under minimal selection stringency (SD8 RBS on AP, proC promoter on CP2).

Evolution summary for Tn7016 TnsA, TnsB, and TnsC Throughout evolution of Tn7016 TnsA, TnsB, and TnsC, selection stringency was modulated by tuning the amount of gIII expressed per integration event, altering the amount of QCascade supplied to guide integration by TnsABC, or requiring multiple integration events per host cell to produce full-length pIII. Adjusting the amount of gIII expressed per integration event was done by adjusting the strength of the ribosome binding site upstream gIII on the AP and by adjusting the strength of the promoter in the transposon encoded by CP2. Adjusting the expression level of QCascade was done by adjusting the strength of the promoter upstream crRNA and QCascade on CP1. Requiring multiple integration events per host cell to produce full-length pIII was done by developing Tns circuits 3 (dual integration system) and 4 (dual integration system with T7 RNAP amplification).

Tn7016 PANCE 1 on Tns circuit 2 was seeded with wild-type TnsA, TnsB, and TnsC and evolved under the conditions for 14 passages under minimal selection stringency (SD8 RBS on AP, proC promoter on CP2, J23119 promoter on CP1). SP from PANCE 1 seeded Tn7016 PACE 1, which was performed for 172 h under moderate selection stringency (SD8 RBS on AP, pro5 promoter on CP2, J23119 promoter on CP1). SP from Tn7016 PACE 1 were pooled at equimolar concentrations and seeded Tn7016 PANCE 2, which was performed for 20 passages under high selection stringency (SD8 RBS on AP, proC promoter on CP2, pro5 promoter on CP1 for 6 passages; then SD8 RBS on AP, pro5 promoter on CP2, pro5 promoter on CP1 for 14 passages). SP from Tn7016 PANCE 2 were pooled and used to seed Tn7016 PACE 2, which was performed for 132 h under moderate selection stringency (sd8 RBS on AP, proC promoter on CP2, J23119 promoter on CP1). Evolved variants from these trajectories did not yield improvements in mammalian editing activity, and thus SP from Tn7016 PACE 2 were not carried on for subsequent evolution.

Following identification of N14-1, a TnsABC variant from Tn7016 PANCE 1 that enabled improved integration in a mammalian context, SP encoding N14-1 were used to simultaneously seed PACEs P7/P8 and PANCE N20. PACE P7 was performed for 108 h at low selection stringency (SD8 RBS on AP, proC promoter on CP2, J23119 promoter on CP1), with only one lagoon (L3) maintaining SP that did not acquire gIII via co-integration. PACE P8 was performed for 132 h at low selection stringency (SD8 RBS on AP, proC promoter on CP2, J23119 promoter on CP1), however gIII acquisition by SP later in PACE required isolation of evolved SP at the 48 h timepoint. PANCE N20 was performed for 10 passages under low selection stringency (SD8 RBS on AP, proC promoter on CP2, J23119 promoter on CP1). Given the prevalence of gIII acquisition during PACEs P7 and P8, SP encoding mammalian-active transposase variants from P8 and N20 confirmed to be AgIII were used to seed Tn7016 PANCE N23 on Tns circuit 3. Tn7016 PANCE N23 was performed for 20 passages under high selection stringency (SD8 RBS on AP/CP1, proD promoter on CP2, dual integration system). Following the development of Tns circuit 4, SP from PANCE N23 were pooled and used to seed Tn7016 PACE P9. PACE P9 was performed for 144 h at moderate selection stringency (SD8 RBS on AP/CP1, proD promoter on CP2, dual integration system with T7 RNAP signal amplification).

Evolution summary for Tn6677 QCascade The QCascade complex was evolved on a circuit adapted from the TnsAB & C evolution. Instead of encoding TnsAB & C on the SP, the entire QCascade complex is encoded on the SP and TnsAB & C expressed by the hosts on the CP plasmid. The Tn6677 QCascade ortholog was evolved on circuit 1 in combination with WT TnsAB & C over 3 rounds of PANCE and 168 h of PACE. After codon-optimization of the QCascade complex, phage propagation was tested on hosts with varying donor promoter and TnsAB & C promoter strengths. Phage de-enriched across all hosts and evolution of the Tn6677 ortholog was not continued.

Evolution summary for Tn7016 QCascade Wild-type human codon optimized Tn7016 QCascade complex was encoded on a SP and propagation was tested on circuits with varying selection stringencies. SP encoding wild-type QCascade de-enriched on all hosts. Phage were then evolved in combination with N14-1 or P8 L5-8 TnsAB and C. Over 30 rounds of PANCE phage propagation improved substantially. PANCE variants are currently tested for integration into the mammalian genome. The evolution of QCascade is continued on circuit 2.

E. coli plasmid editing assay For assessing the activity of evolved Tn7016 TnsABC variants, S2060 E. coli encoding pTarget, pDonor, and CP (with Tn7016 crRNA and TniQ-Cascade) were made chemically competent and transformed with pTnsABC encoding the TnsABC variant under an arabinose inducible promoter. Following transformation, cells were recovered for 1 h at 37° C. in SOC media, plated on LB agar containing the appropriate maintenance antibiotics and 10 mM arabinose, and incubated for 24 h at 37° C. Importantly, cells were plated at a density where single colonies were still distinguishable after growth. Following 24 h incubation, cells were scraped, resuspended, and plasmid DNA was isolated using a QIAprep spin miniprep kit (Qiagen) according to manufacturer instructions. For assessing the activity of dSpCas9 fusions, the protocol was performed as above except the CP encoded a SpCas9 sgRNA and Tn7016 TnsABC, and E. coli were transformed with a pCas-TniQ/TnsC plasmid that contained dSpCas9 fused to TniQ or TnsC under arabinose inducible expression. The “-unfused TnsC” conditions used a CP lacking TnsC, and the “-fused TnsC” conditions used a pCas-TnsC lacking TnsC.

qPCR quantification of integration events in E. coli. qPCR quantification of integration was performed as previously described (Klompe, et al., Nature 2019) with the following modifications. Isolated plasmid DNA was diluted 100-fold and used as template for a 20 μL qPCR as follows: 0.1 μL each 100 μM primer, 10 μL 2×Q5 master mix (NEB), 0.2 μL 100×SYBR Gold (Thermo Fischer Scientific), 4 μL plasmid template or standard, 5.6 μL water. A standard is prepared of varying dilutions of unintegrated to synthetically created integrated plasmid. qPCRs were run as follows: (98° C. for 20 s, 60° C. for 20 s, 72° C. for 20 s, capture)×40. The amount of integrated target plasmid was determined by qPCR with primer pairs spanning the transposon end: pTarget junction (integration), and total amount of target plasmid was determined by qPCR with primer pairs binding the pTarget backbone (reference). A standard curve for % integration was generated by plotting ΔC_{q} vs. log (% integration), where ΔC_{q} is the C_{q} difference between integration and reference reaction. Integration efficiencies for experimental conditions were determined by interpolating the standard curve.

PCR and Sanger sequencing analysis of dSpCas9-TniQ/InsC transposition products. PCR and Sanger sequencing analysis of integration was performed as previously described (Klompe, et al., Nature 2019) with the following modifications. 1 μL isolated plasmid DNA was used as template for a 25 μL PCR containing 0.25 μL each 100 μM primer, I2.5 μL 2× Phusion U master mix, and 11 μL water. PCRs were run as follows: 98° C. for 2 min, then 35 cycles of [98° C. for 15 s, 64° C. for 20 s, 72° C. for 30 s], followed by a final 72° C. extension for 2 min. Primer pairs were designed to span transposon end: pTarget junctions for T-RL products (Amplicons 1 and 2) and T-LR products (Amplicons 3 and 4). PCR amplicons were resolved by 1-2% agarose gel electrophoresis and visualized by staining with ethidium bromide. Bands with sizes corresponding to expected transposition products were extracted and purified by QIAquick Gel Extraction Kit (Qiagen), and samples were submitted to Quintara Biosciences for Sanger sequencing analysis.

HEK 293T transfection and genomic DNA extraction. HEK 293T cells (ATCC CRL-3216) maintained in Dulbecco's Modified Eagle's Medium plus GlutaMax (Thermo Fisher Scientific) supplemented with 10% (v/v) FBS at 37° C. with 5% CO₂ were seeded on 48-well plates (Corning) at a density of ˜42,500 cells/well. 16-20 h after seeding, cells were transfected at approximately 80-85% confluency with 50 ng each of plasmids encoding Cas6, Cas7, Cas8, and TniQ, 300 ng of pDonor/crRNA plasmid, 2 ng of plasmid target (if included), 150 ng of plasmid encoding TnsA-B, 150 ng of plasmid encoding TnsC, and 1.5 μL of Lipofectamine 2000 (Thermo Fischer Scientific). Alternatively, 75 ng of pQCascade plasmid expressing Cas6, Cas7, Cas8, and TniQ split by P2A linkers was used in place of the 4 monocistronic plasmids for QCascade expression. Transfected cells were cultured for 3 days post-transfection before the media was removed, cells were washed with 1×PBS solution (Thermo Fisher Scientific), and genomic DNA was extracted by addition of 50 μL lysis buffer (10 mM Tris-HCL (pH 8.0), 0.05% SDS, 25 μg/mL Proteinase K (Thermo Fisher Scientific)) followed by heat inactivation of Proteinase K by incubation at 80° C. for 30 min. Genomic DNA was stored at −20° C. until further use.

### High-Throughput Sequencing Quantification of Integration Events

For amplicon sequencing of DNA insertion products, donors were constructed based on site of interest such that inserted and un-inserted sites would amplify to the same size. To do so, the reverse primer binding site that binds to the genomic DNA 3′ of the expected integration site was inserted into the donor DNA such that the distance from expected integration site to the primer binding site in the integrated donor is equal to the expected integration site to the primer binding site in the unintegrated genome.

Genomic and plasmid target sites were amplified with primers targeting the region of interest and containing the appropriate universal Illumina forward and reverse adapters. PCR 1 reactions contained 0.125 μL each of 100 μM forward and reverse primers, 5 μL genomic DNA extract, 25 μL of 2× Phusion U Hot Start mix (Thermo Fisher Scientific), and 19.75 μL water. PCR 1 conditions: 98° C. for 2 min, then 27 cycles of [98° C. for 15 s, 62° C. for 20 s, 72° C. for 30 s], followed by a final 72° C. extension for 2 min. PCR products were verified by comparison with DNA standards (Quick-Load 2-Log Ladder; New England BioLabs) on a 2% agarose gel supplemented with ethidium bromide. Unique Illumina barcoding primers were subsequently appended to each PCR 1 sample in a second PCR reaction (PCR 2). PCR 2 reactions used 1.25 μL each of 10 μM forward and reverse Illumina barcoding primers and 1 μL of unpurified PCR 1 reaction product in 25 μL of Phusion U Hot Start mix prepared according to the manufacturer's protocol (Thermo Fisher Scientific). PCR 2 conditions: 98° C. for 2 min, then 10 cycles of [98° C. for 15 s, 61° C. for 20 s, 72° C. for 30 s], followed by a final 72° C. extension for 2 min. PCR products were pooled and purified by electrophoresis with a 2% agarose gel using a QIAquick Gel Extraction Kit (Qiagen Inc.) eluting with 30 μL H₂O. DNA concentration was quantified with a Qubit dsDNA High Sensitivity Assay Kit (Thermo Fisher Scientific) and sequenced on an Illumina MiSeq instrument (paired-end read—R1: 220 cycles, R2: 0 cycles) according to the manufacturer's protocols.

General HTS data analysis. Sequencing reads were demultiplexed using the MiSeq Reporter (Illumina) and fastq files were analyzed using Crispresso2 to align to predicted sequences of uninserted, T-RL products, or T-LR products. Integration efficiency was measured as number of reads aligned to integrated products/total aligned reads.

### Example 1

### Evolution of TnsA, TnsB, and TnsC from Tn6677

Initial PANCE campaigns were conducted under 16 conditions. 4 host E. coli strains were used, testing 2 AP architectures with 2 different target sites upstream gIII. AP architecture A had a synthetic “junk” sequence between the Cascade target site and integration site, whereas AP architecture B had a terminator between the Cascade target site and integration site to prevent basal gIII expression in the absence of integration. Evolutions included SP encoding either fused or unfused TnsAB. Evolutions were conducted at both 30° C. and 37° C. to assess which would be the optimal temperature for future INTEGRATE evolution campaigns.

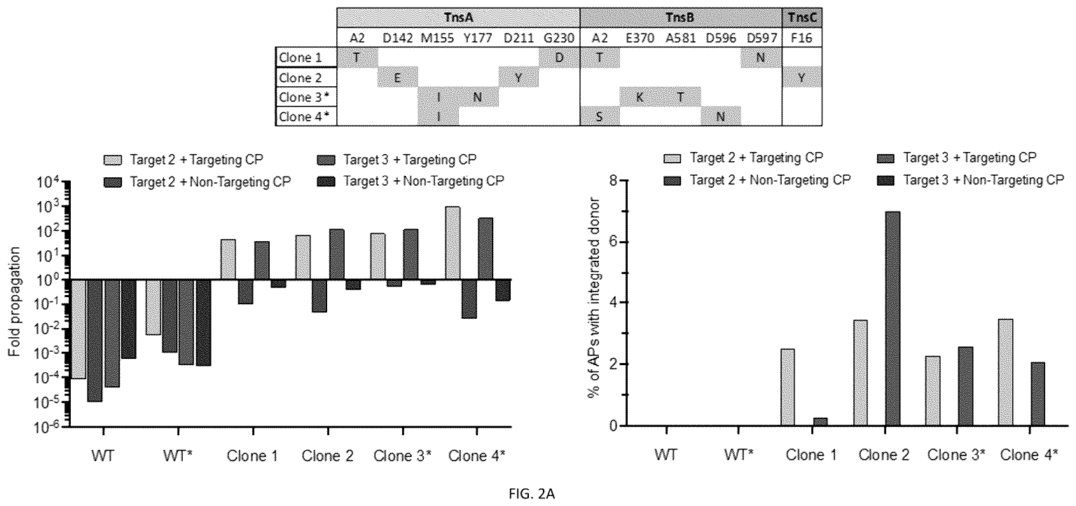

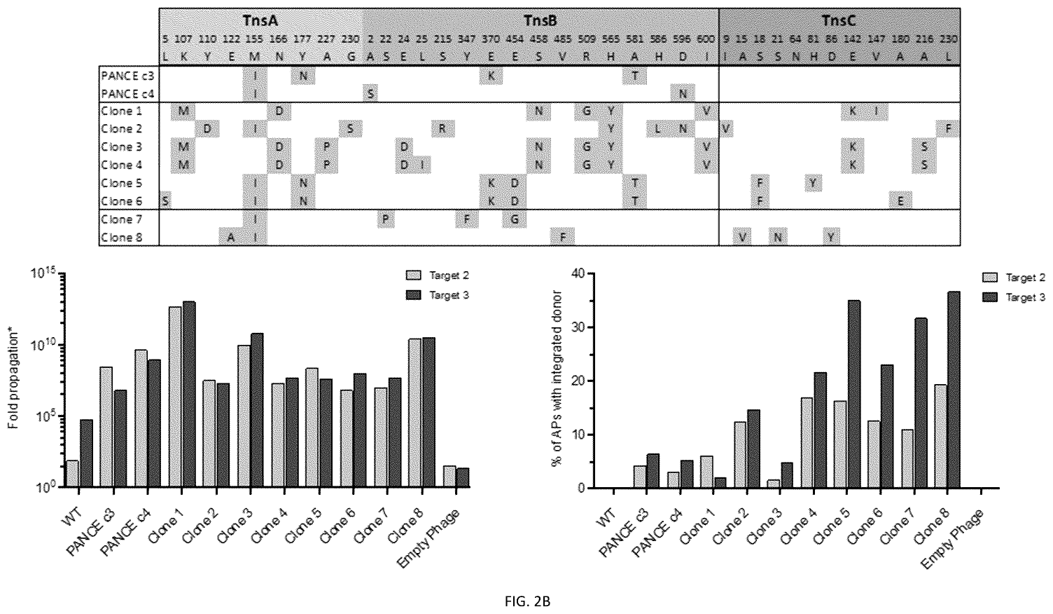

Beginning in P10, SP acquired gIII via recombination, though these SP failed to outcompete non-recombinant INTEGRATE SP, likely due to high activity of evolved variants on the selection circuit. Following P15, SP were cloned into new AgIII backbones to seed future evolutions. Clones were sequenced from the 4 best performing lagoons. Variants from PANCE 1 (clones 1-4) propagated more efficiently on the selection circuit, and this propagation correlated with integration of the donor at the AP as measured by qPCR (FIGS. 2A and 2B). As a result of gIII recombinants arising in PANCE 1, SP encoding full length TnsAB-TnsC were subcloned into new SP backbones known to lack gIII. These SP seeded PACE 1.

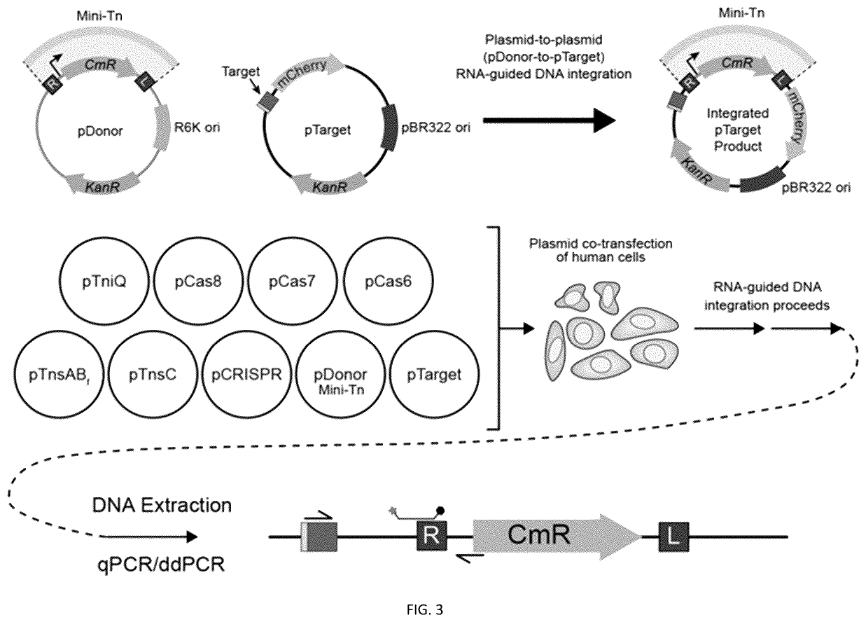

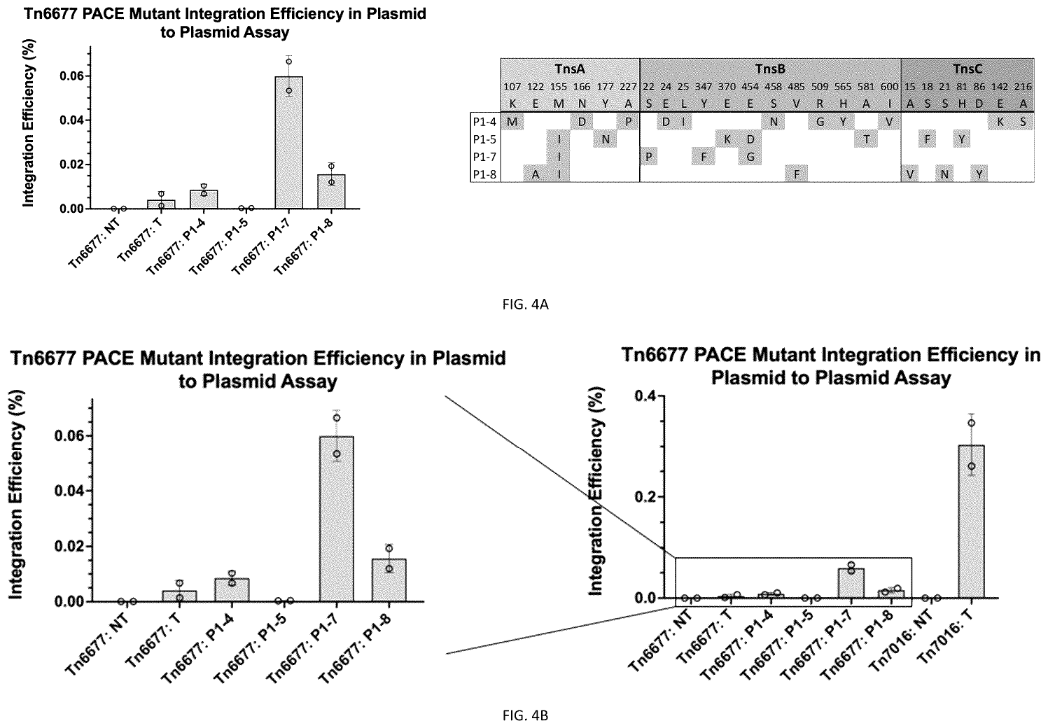

To assess the efficiency of evolved Tn6677 TnsA, TnsB, and TnsC variants, plasmid-to-plasmid integration assays were performed in HEK293T cells (FIG. 3). Evolved TnsABC variants were cloned into mammalian expression vectors and co-transfected with expression vectors for QCascade components (pCas8, pCas7, pCas6, pTniQ, pCRISPR) along with a donor transposon (pDonor Mini-Tn) and plasmid target (pTarget). Following incubation for 72 hours, cells were lysed and integrated target plasmid was measured by qPCR with a probe for integration 49 bp downstream of the target site. Tn6677 PACE 1 variants demonstrate up to 15-fold increased plasmid to plasmid editing in mammalian cells (FIGS. 4A-4B).

### Example 2

### Evolution of TnsA, TnsB, and TnsC from Tn7016

Tn7016, a transposon encoded by Pseudoalteromonas sp. S983 that has a higher activity in a mammalian context than WT was cloned into the INTEGRATE PACE circuit for subsequent evolutions. Initial PANCE was conducted on Tns Circuit 2 with 2 AP architectures, as previously described for Tn6677. Following PANCE, SP were evolved in PACE (all with AP architecture B). SP titers decreased initially, but rescuing lagoons with pooled SP enabled several lagoons to maintain titers through a lagoon flow rate of 3 v/h (typically the highest flow rate conducted in PACE).

To assess whether PACE-evolved variants enabled improved activity in E. coli, variants were cloned into inducible expression vectors (pTnsABC) and transformed into host E. coli encoding QCascade, a donor transposon, and a plasmid target. Integration in either orientation downstream the target site (T-RL or T-LR) was monitored by qPCR with primers specific to the transposon: pTarget junction and percent integration was determined by normalizing integration to a qPCR with primers specific to the pTarget backbone.

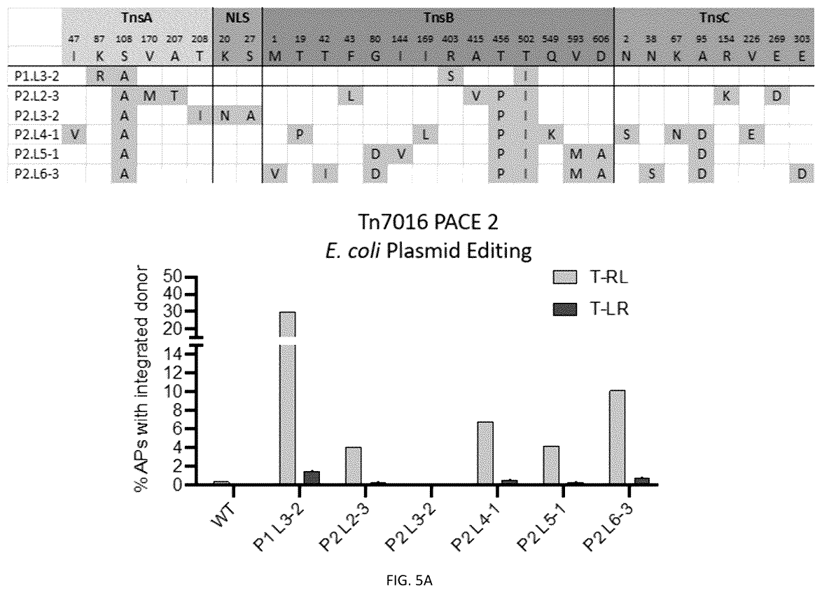

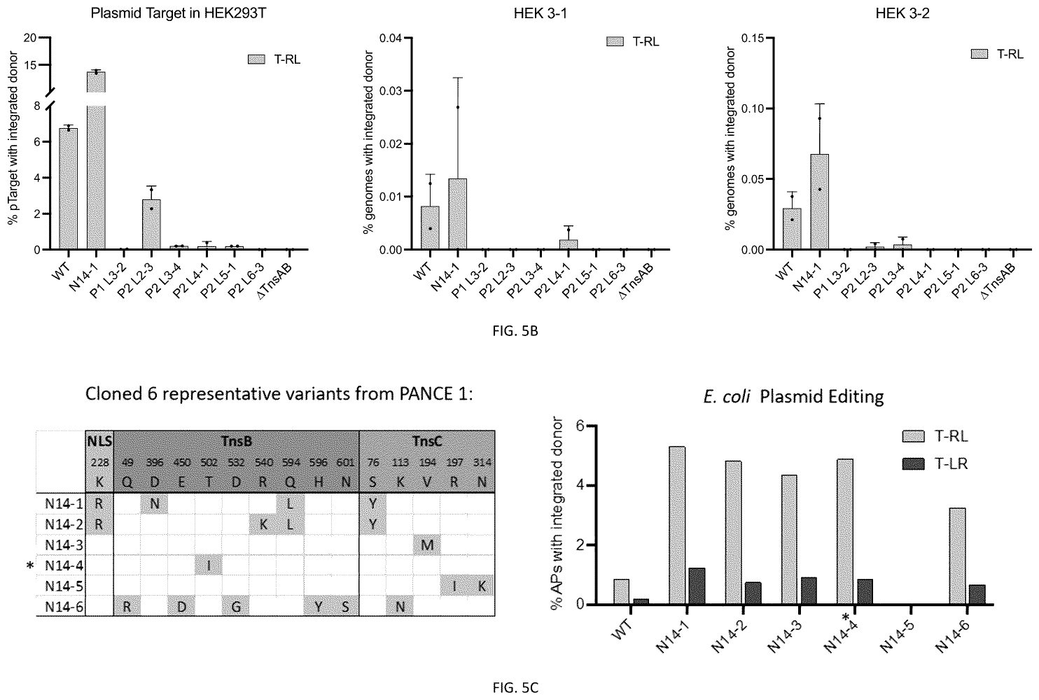

Tn7016 PACE 1 variants were subject to a PANCE and subsequent PACE under higher selection stringencies (reducing the strength of the promoter encoded in the transposon). PACE 2 variants improved transposition in E. coli compared to WT TnsABC, but efficiencies do not exceed best PACE 1 variant (P1 L3-2) (FIG. 5A). In addition to plasmid editing, PACE 2 variants were tested at 2 mammalian genomic targets within the HEK3 locus. While mammalian genome editing was detectable, PACE 2 did not enable improved activity in a mammalian cells (FIG. 5B).

While PACE 1 and 2 generated variants with improved activity in a bacterial context, these evolutions did not improve editing in a mammalian context. To assess whether the loss of genotypic diversity due to variant pooling in PACE 1 resulted in a loss of mammalian compatible variants, representative variants from passage 13 of Tn7016 PANCE 1 (N14-1 through 6) were characterized. These variants showed improved integration efficiencies in E. coli and at plasmid and genomic targets in HEK293 Ts (FIGS. 5C-5E).

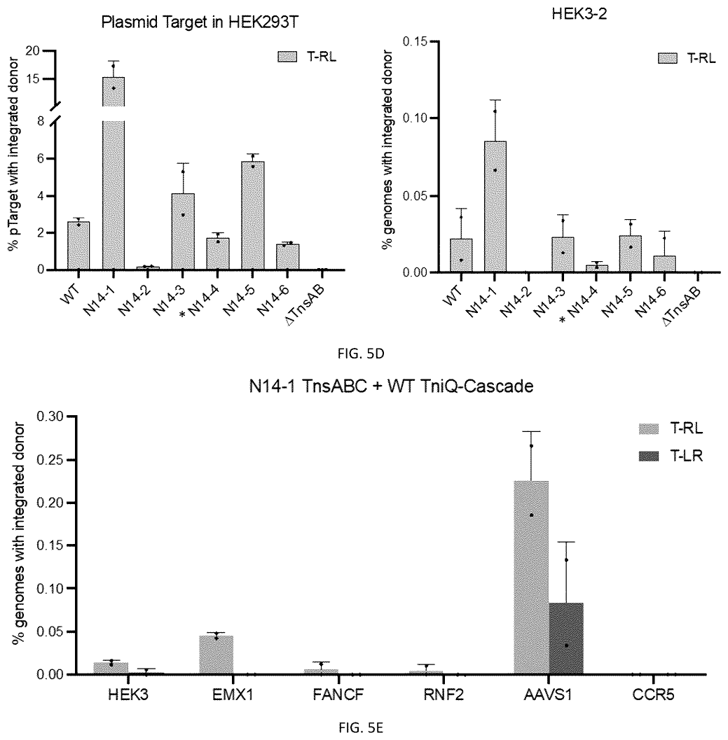

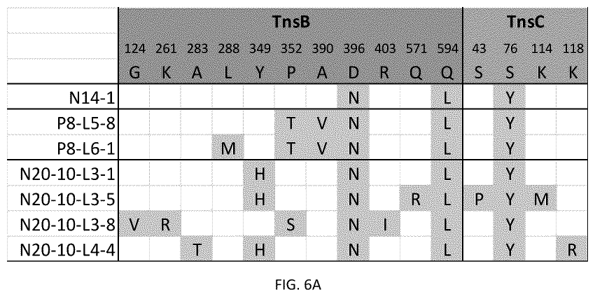

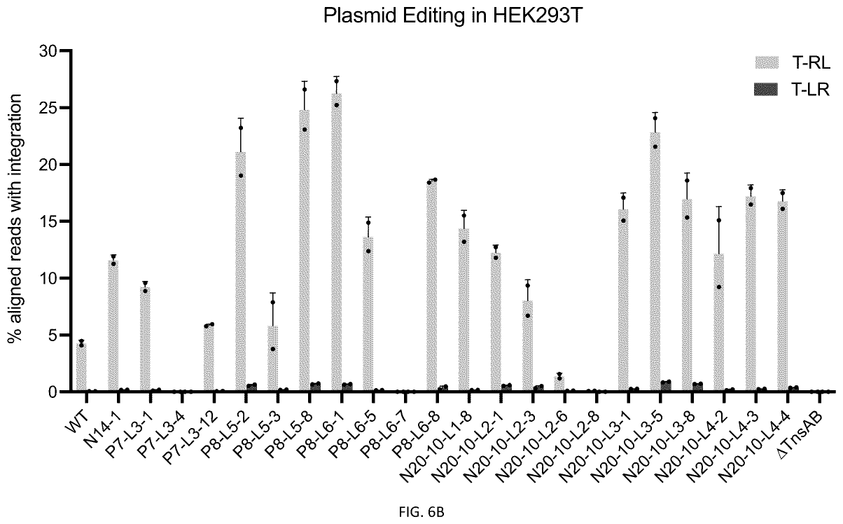

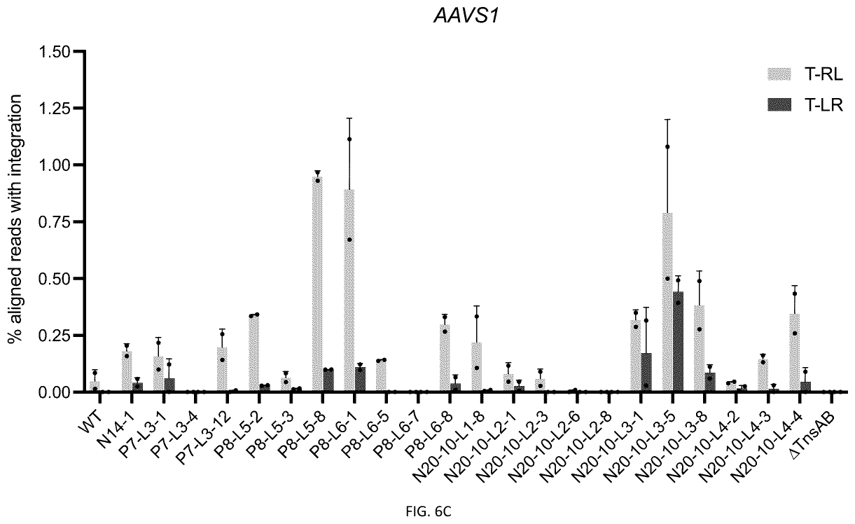

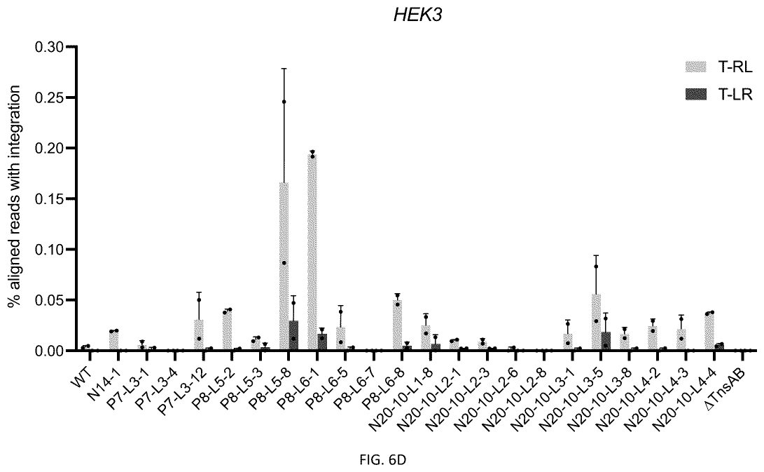

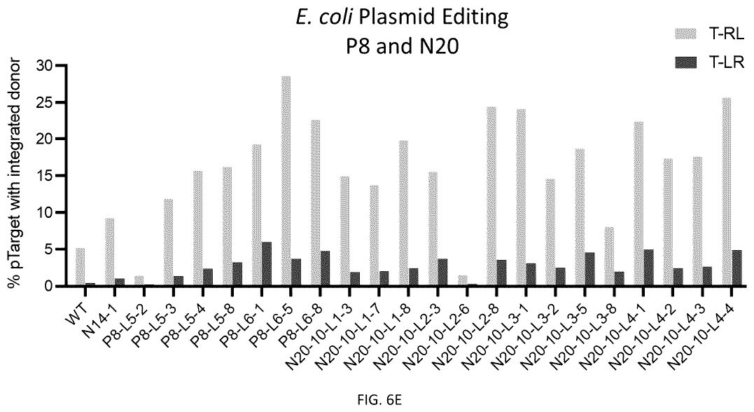

To enable generation of variants with further increased efficiencies in a mammalian context, N14-1 from PANCE 1 was used to seed PACE (P7/P8) and PANCE (N20), and these evolutions were conducted simultaneously. Variants from PACE P8 and PANCE N20 improved editing efficiencies in HEK293 Ts (FIGS. 6B-6D) Genotypes enabling highest editing efficiencies in a mammalian context are shown in FIG. 6A. PACE P8 and PANCE N20 enable variants with improved editing efficiencies in E. coli (FIG. 6E).

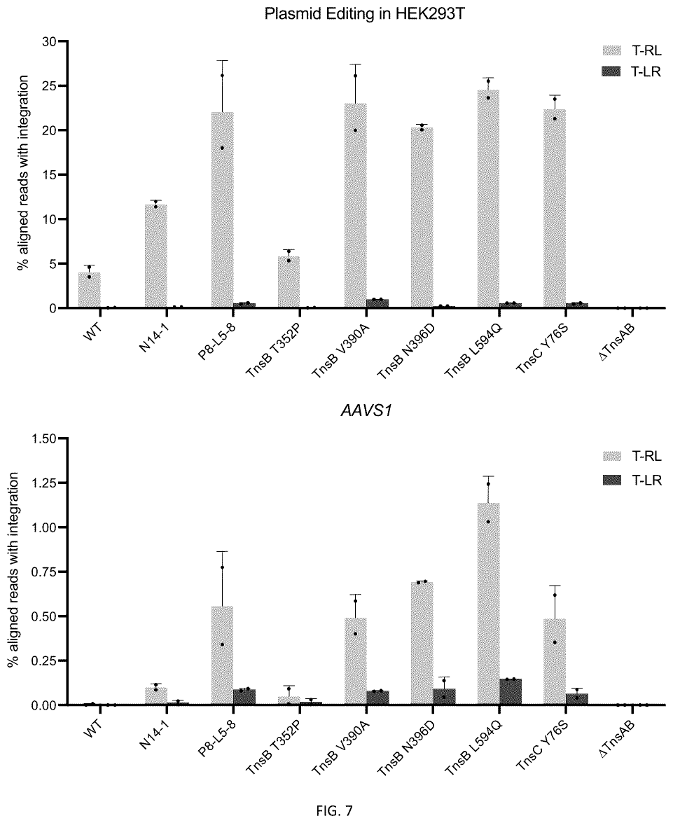

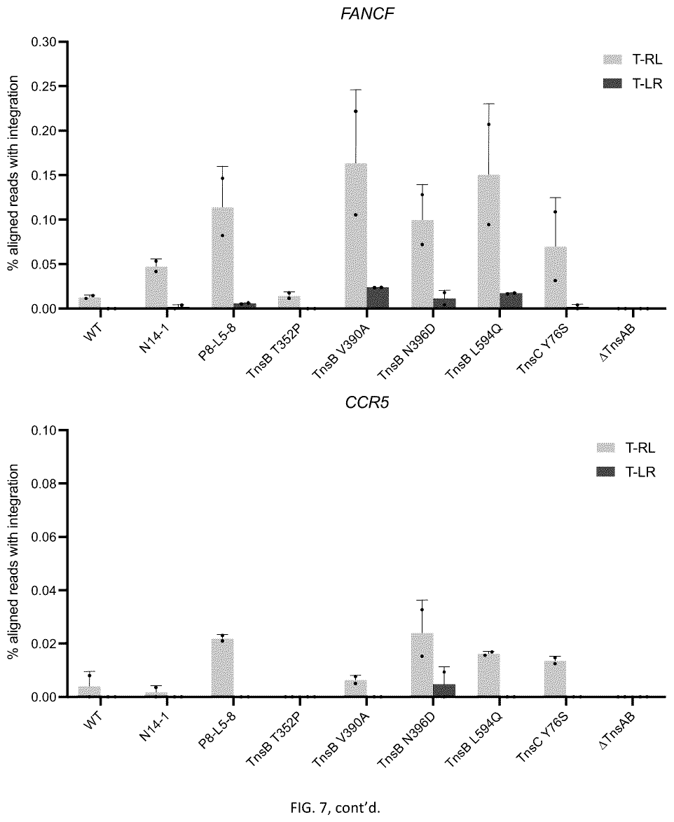

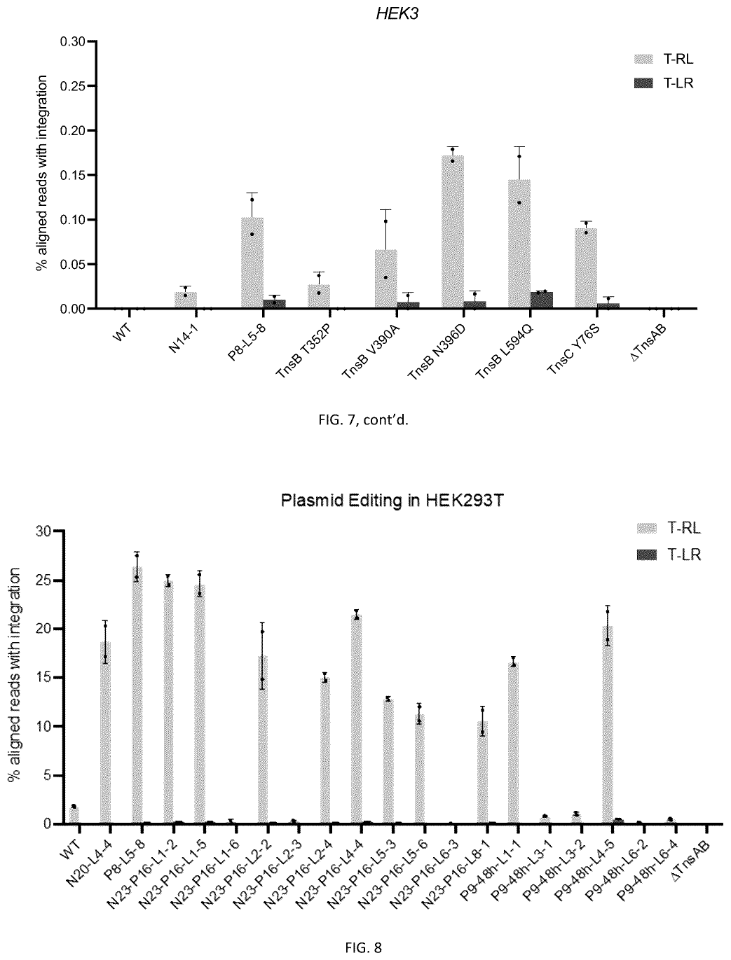

P8 L5-8 demonstrated improved efficiencies across all genomic loci tested. To identify mutations responsible for activity, variants in which the individual mutations were restored to wild-type identity were tested for integration efficiency (FIG. 7). P352T in TnsB was identified as a key mediator of mammalian activity.

Further evolution of TnsABC was complicated by gIII acquisition by SP encoding hyperactive TnsABC variants. Co-integration is a known byproduct of Tn7-like transposases, wherein deficient TnsA endonuclease activity leads to replicative transposition. In the context of PACE, off-target co-integration of a previously integrated AP substrate into the SP genome results in gIII acquisition. gIII acquisition by the SP poisons further evolution efforts, as gIII expression is no longer contingent on the activity of the protein of interest encoded by the SP. Circuit 3 was used to reduce the risk of co-integration.

As a result of requiring at least 2 integration events per infection to produce full length pIII, the selection stringency imposed on SP was significantly higher than previously imposed by Tns Circuit 2. To reduce the selection stringency, a T7 RNAP signal amplification was incorporated for production of N term gIII-NpuN, where a single integration event on the CP promotes T7 RNAP expression, which subsequently promotes N term gIII-NpuN. This reduced selection stringency enables selection of SP in PACE as opposed to PANCE, facilitating more rapid evolution of increased transposition activity. Variants evolved on Circuit 3 (“split gIII circuit”) demonstrated improved propagation on Circuit 4 (“split gIII+T7 circuit”).

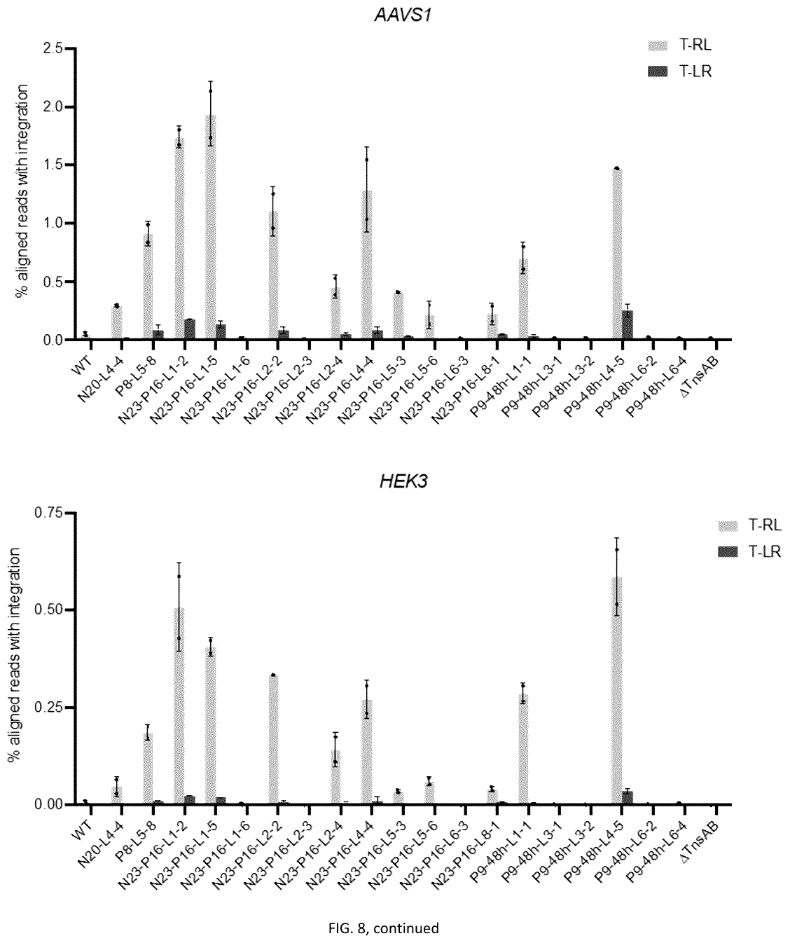

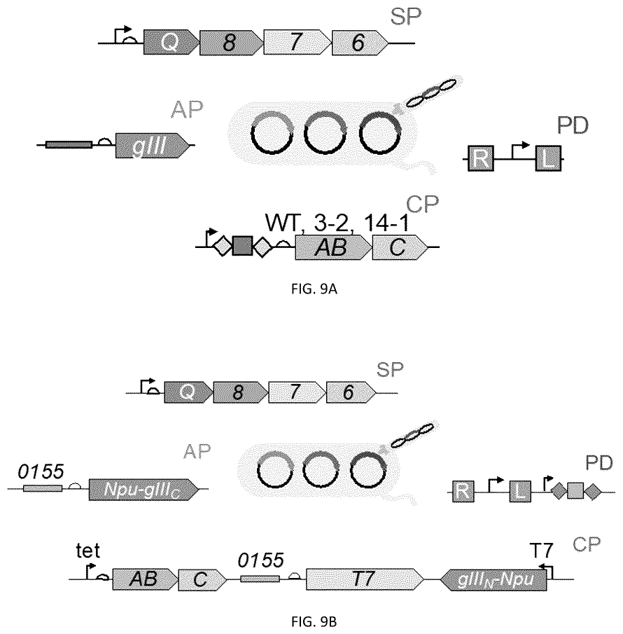

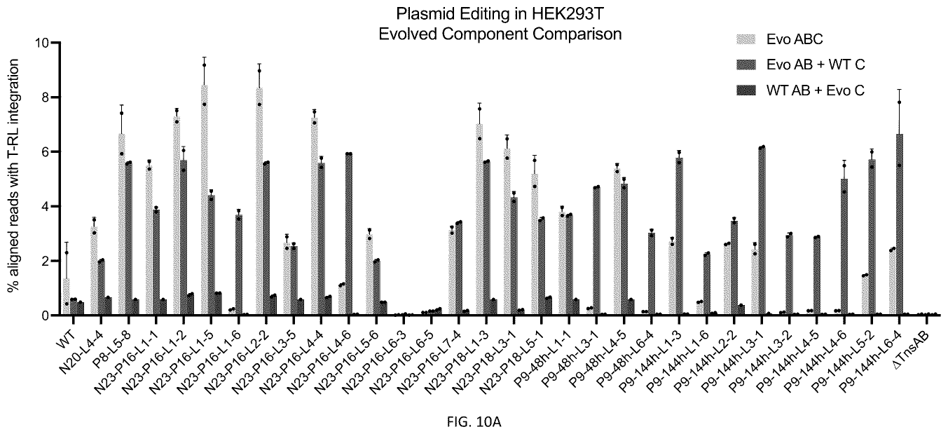

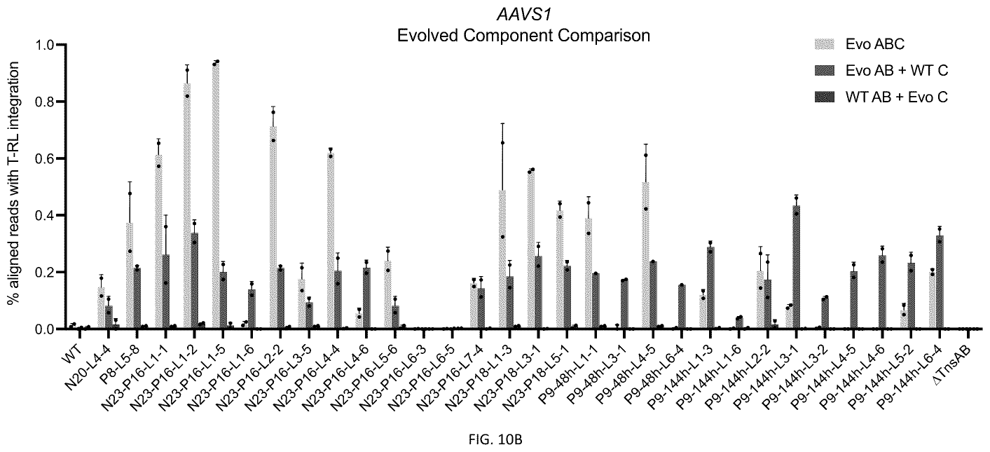

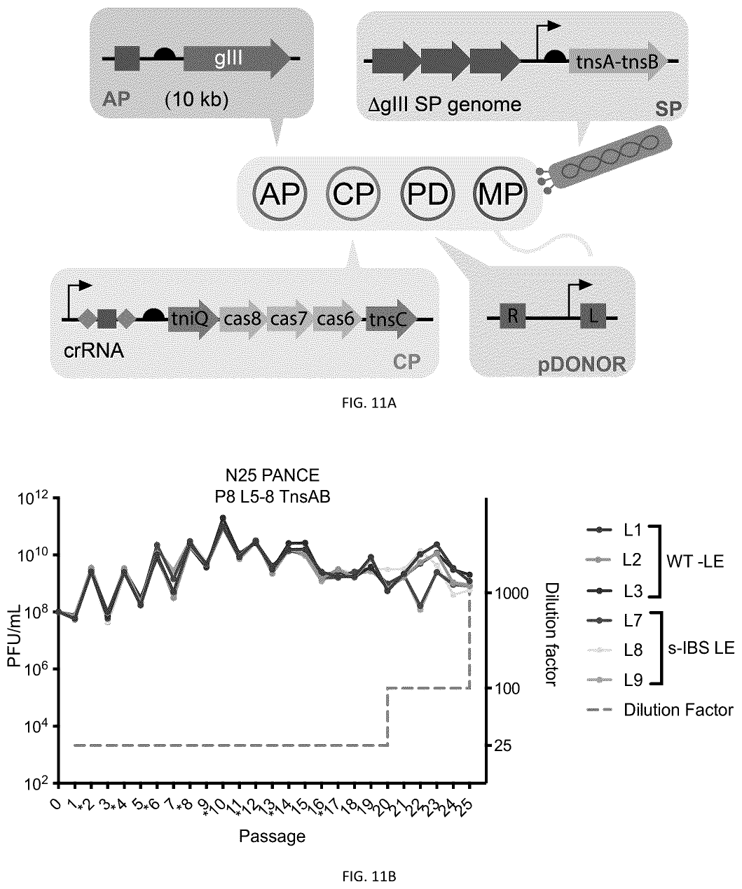

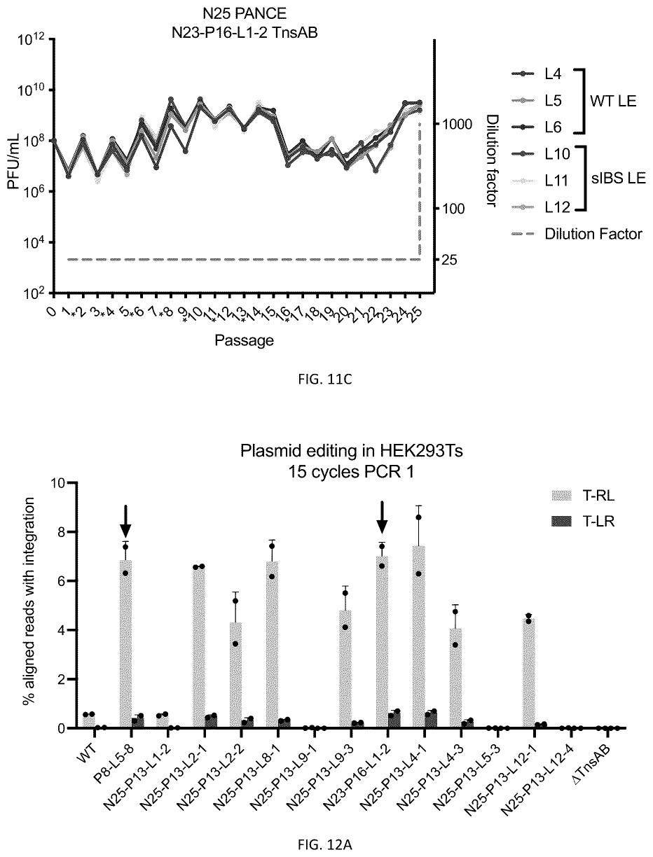

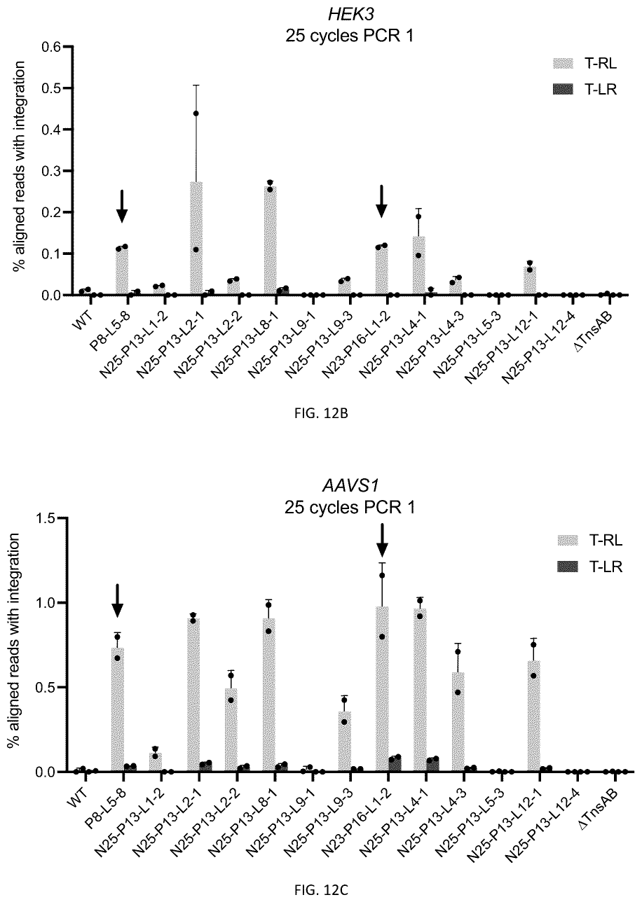

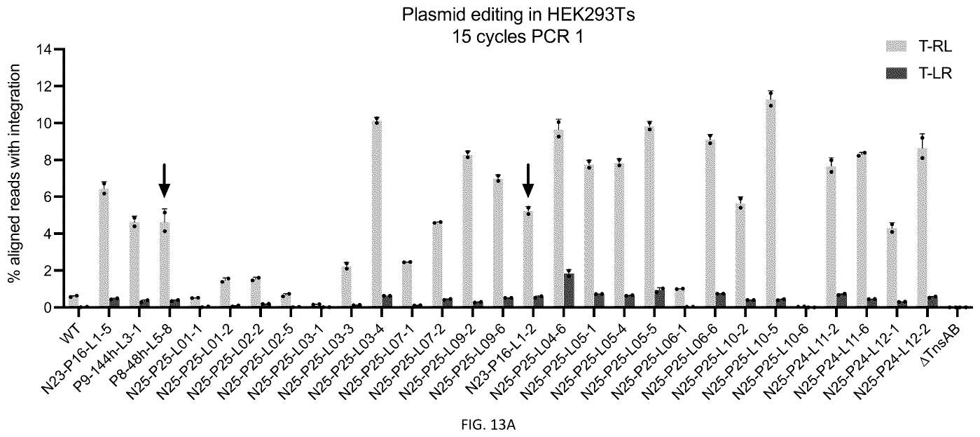

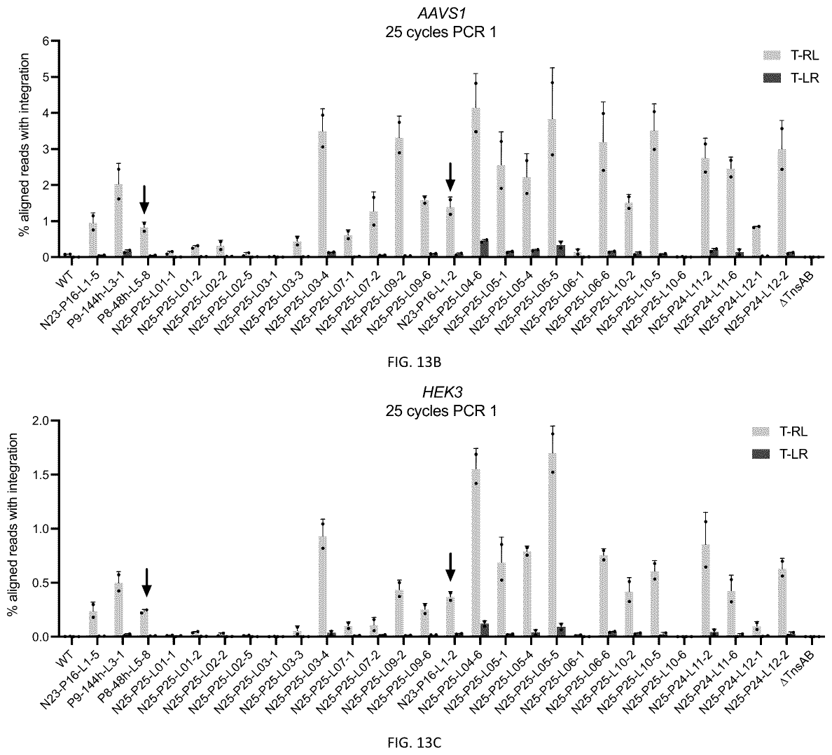

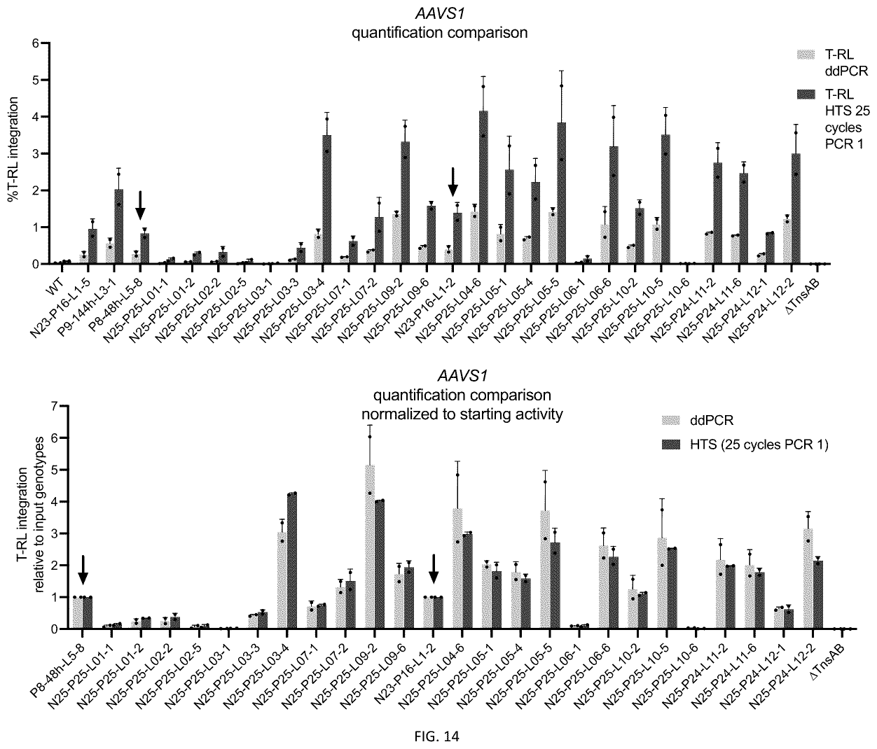

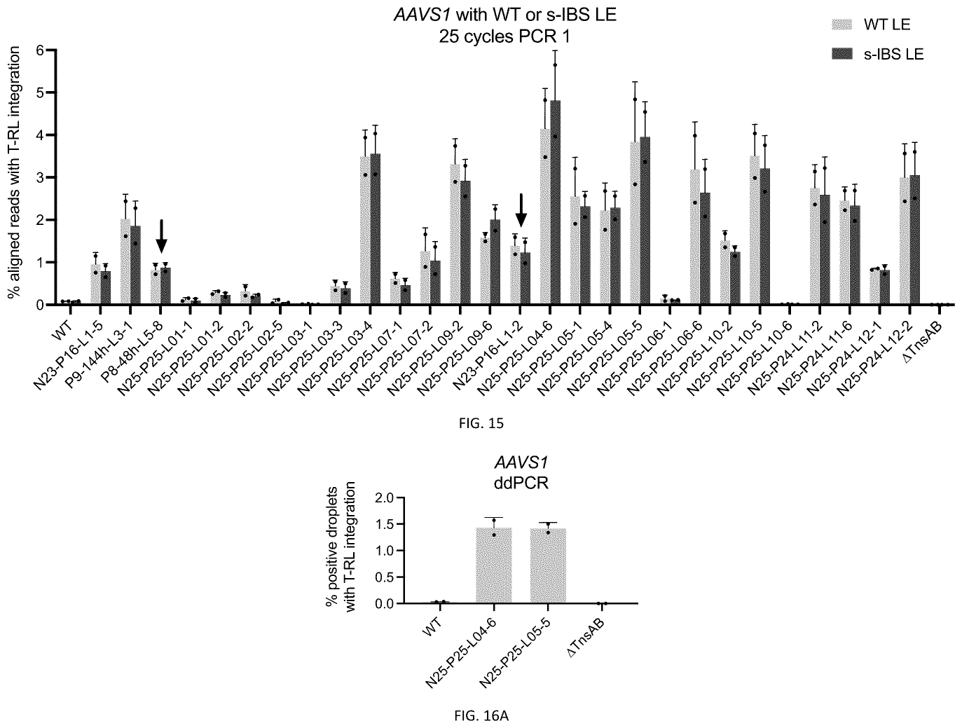

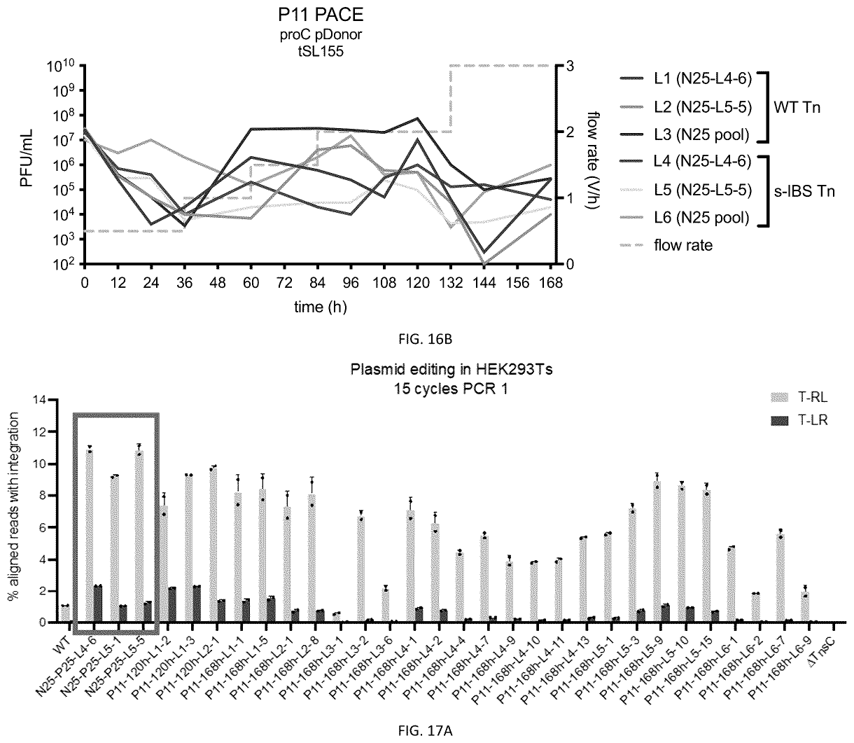

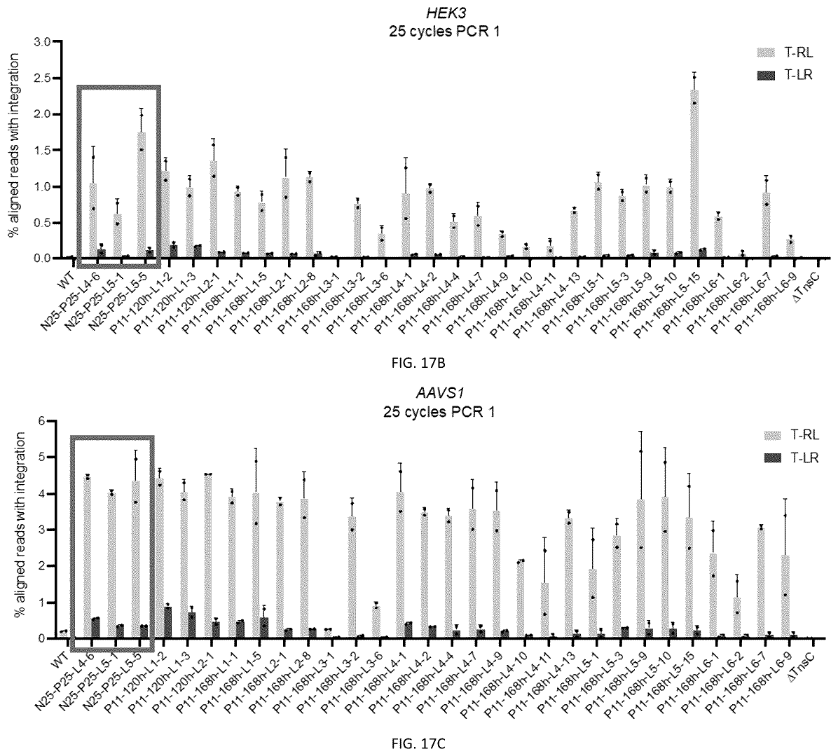

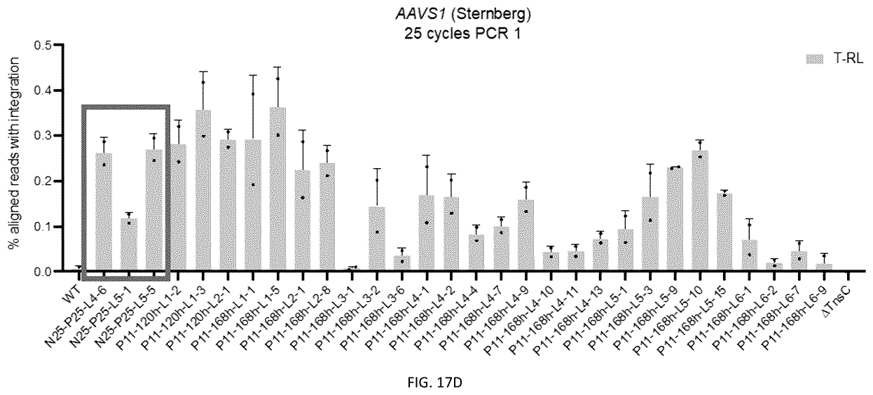

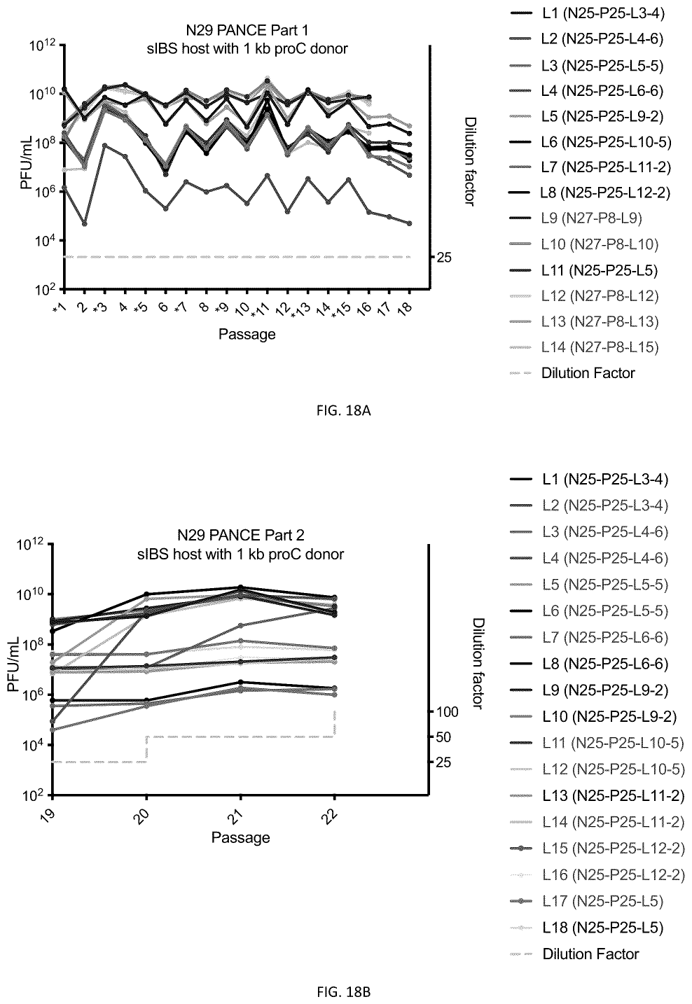

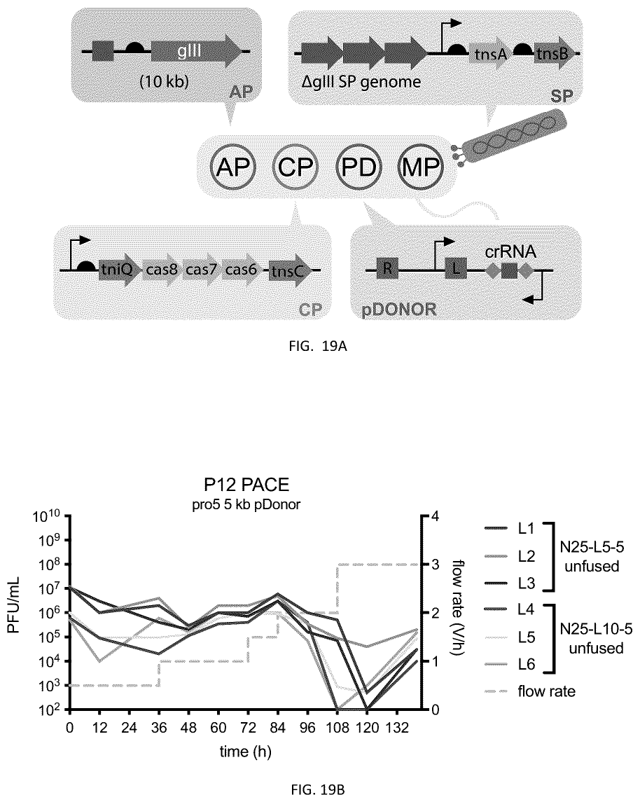

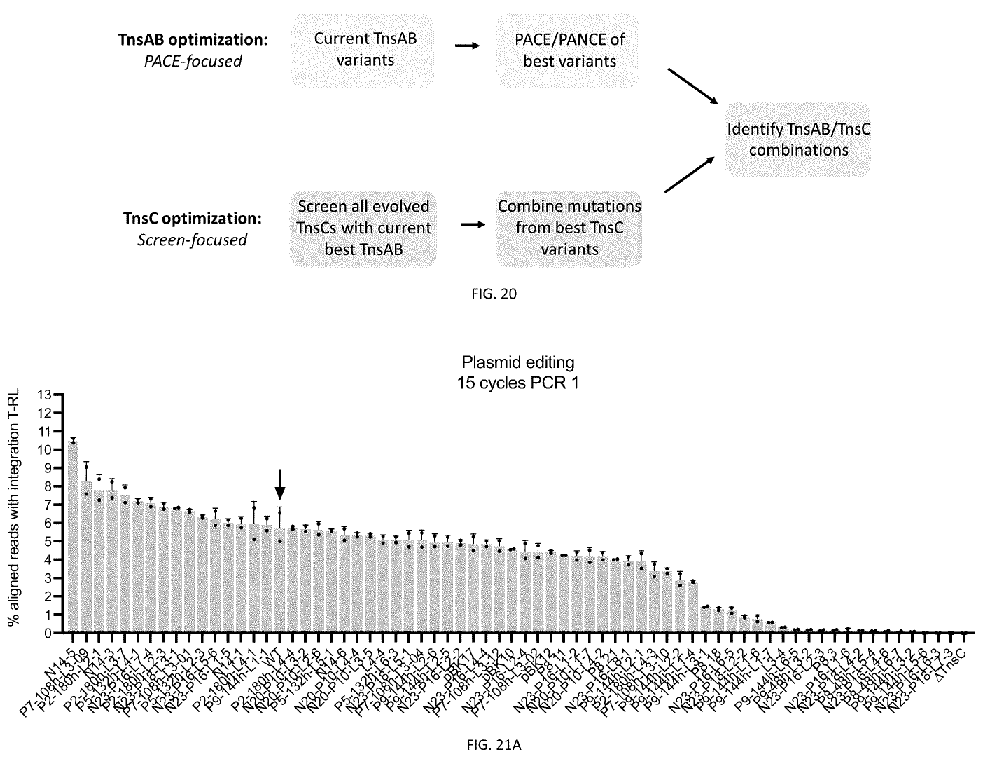

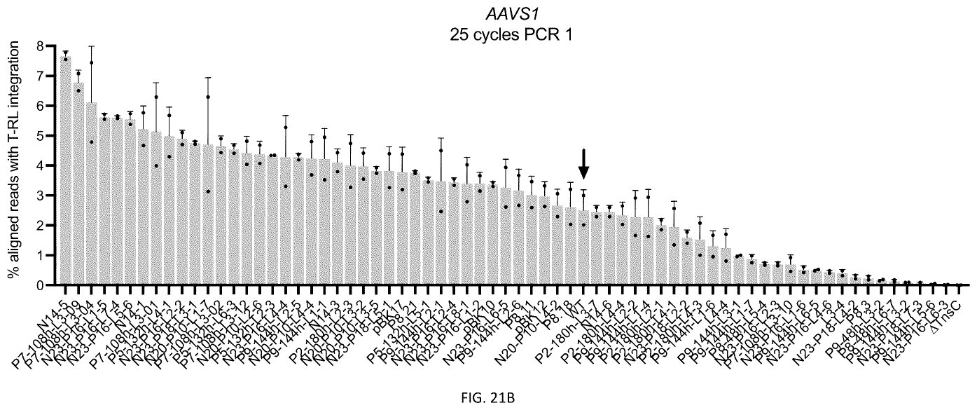

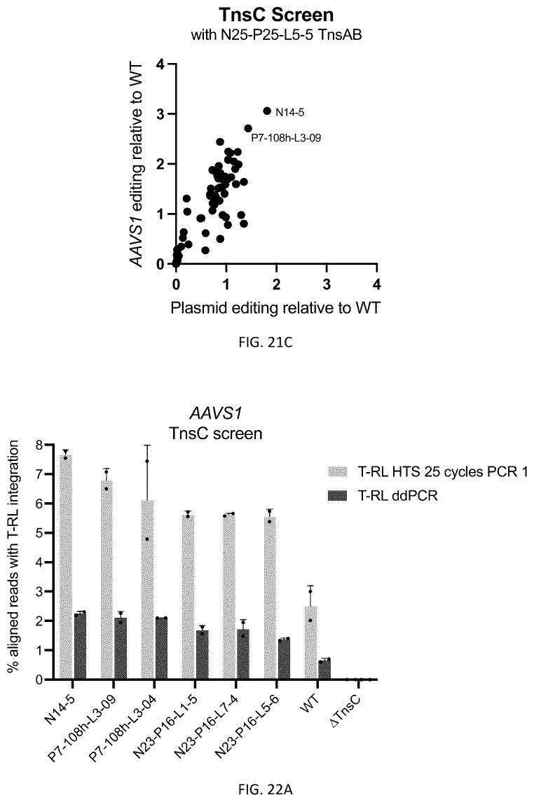

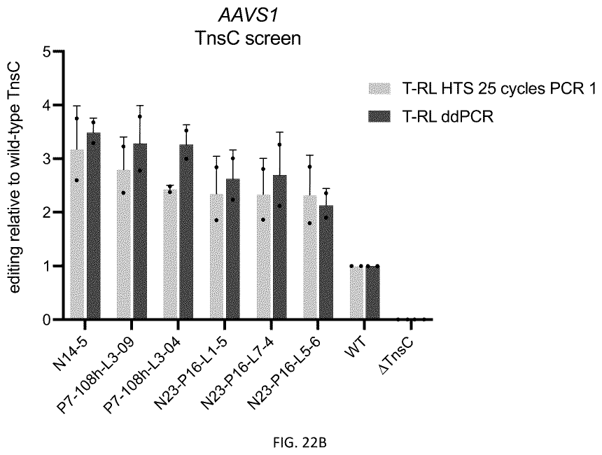

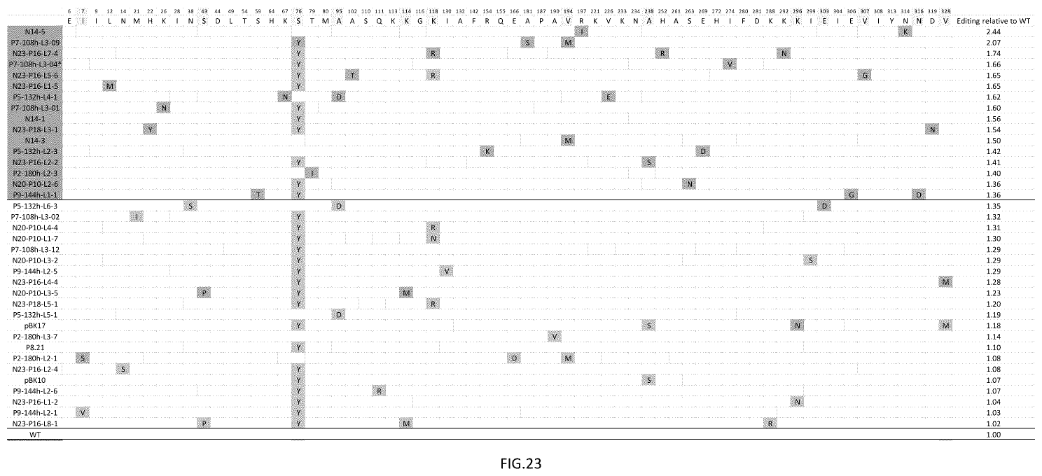

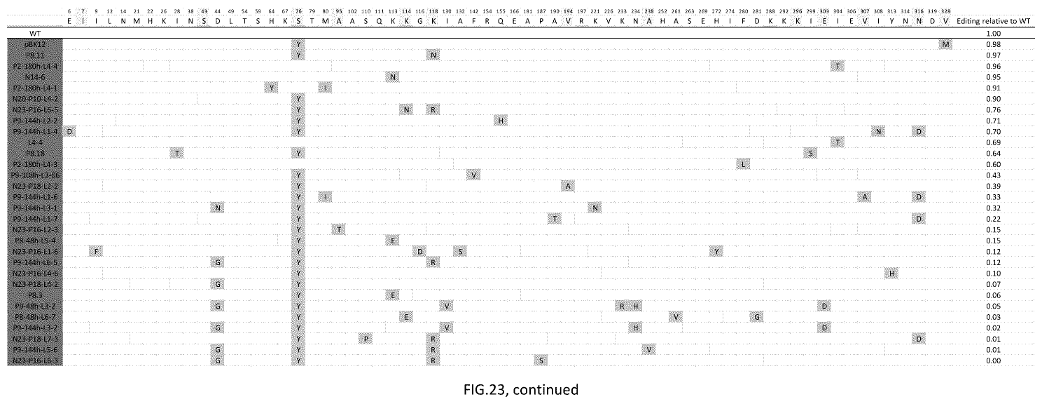

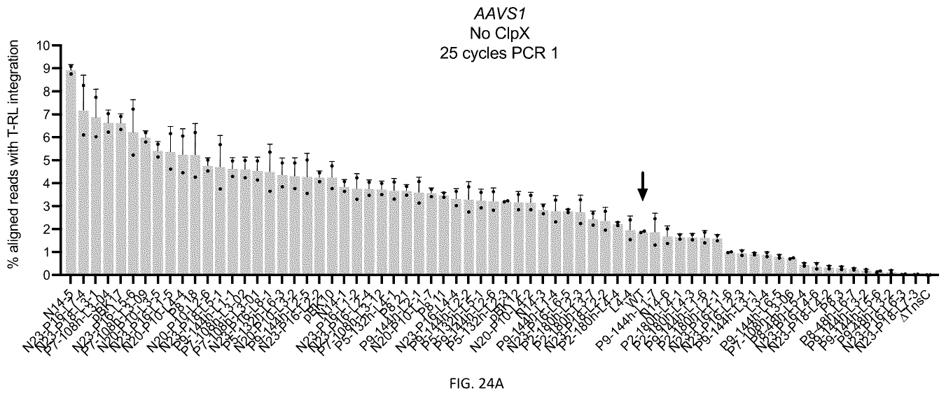

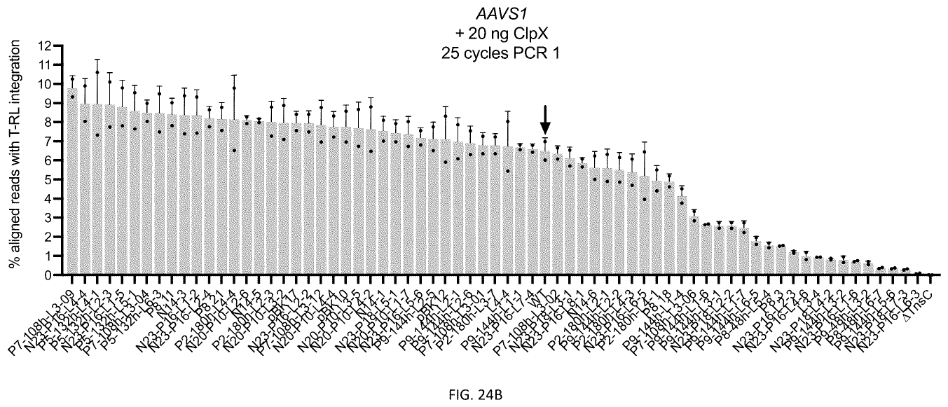

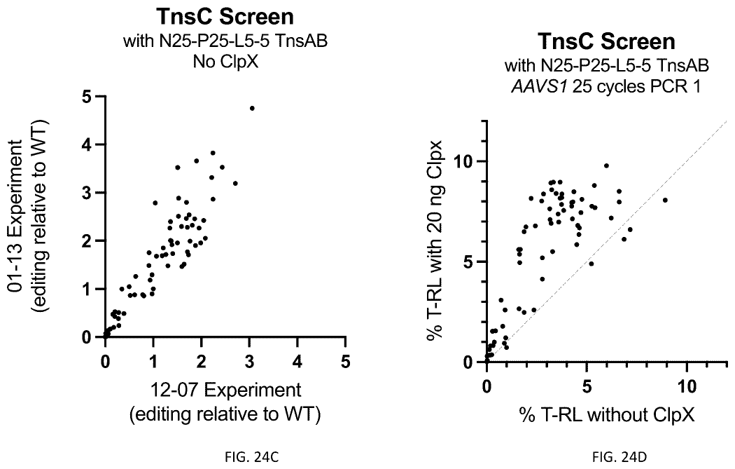

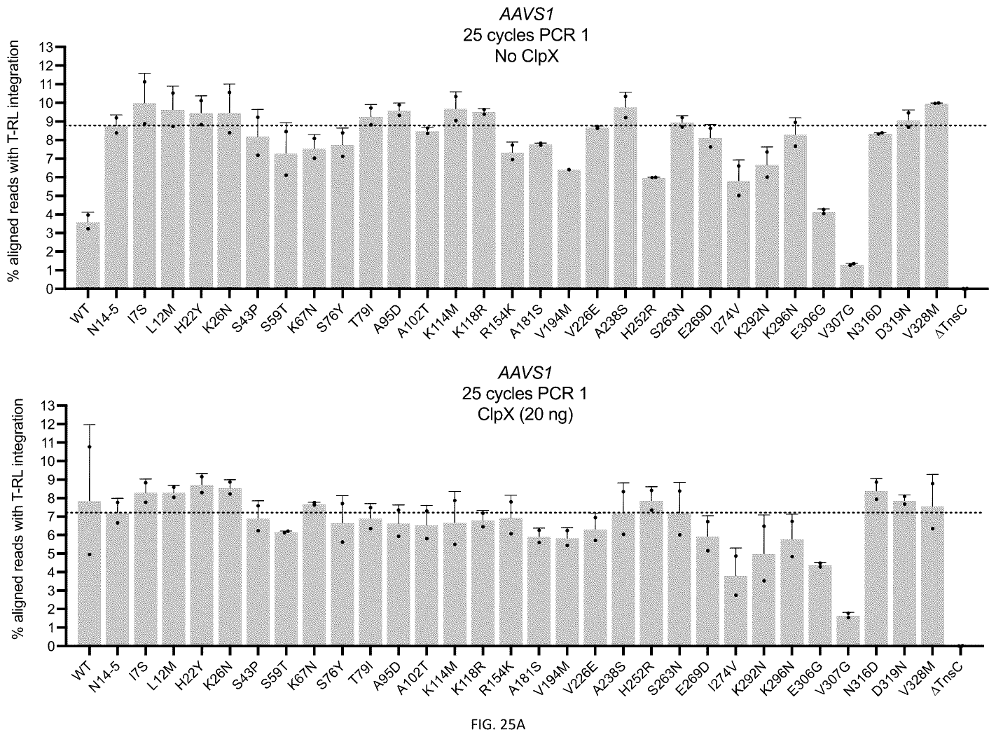

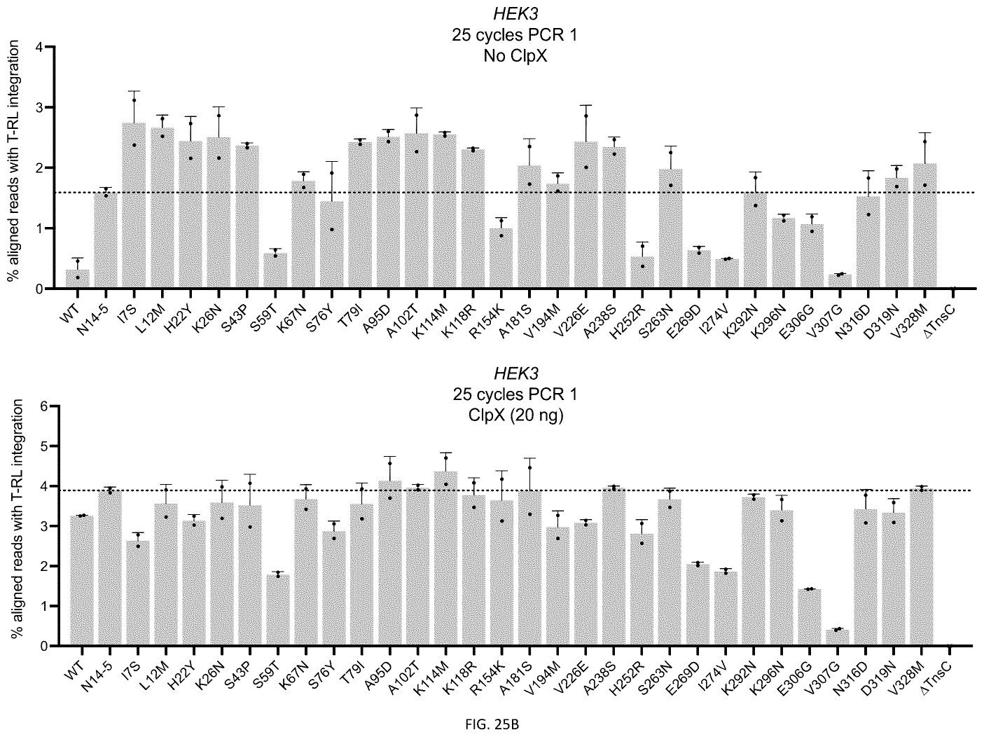

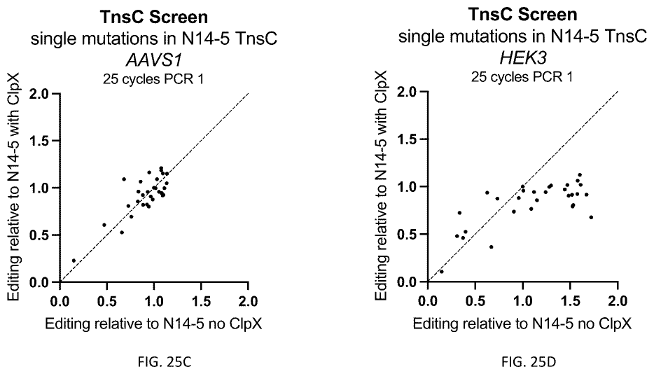

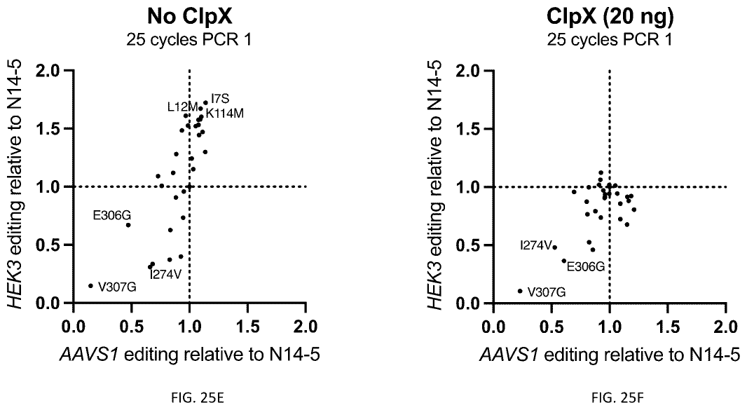

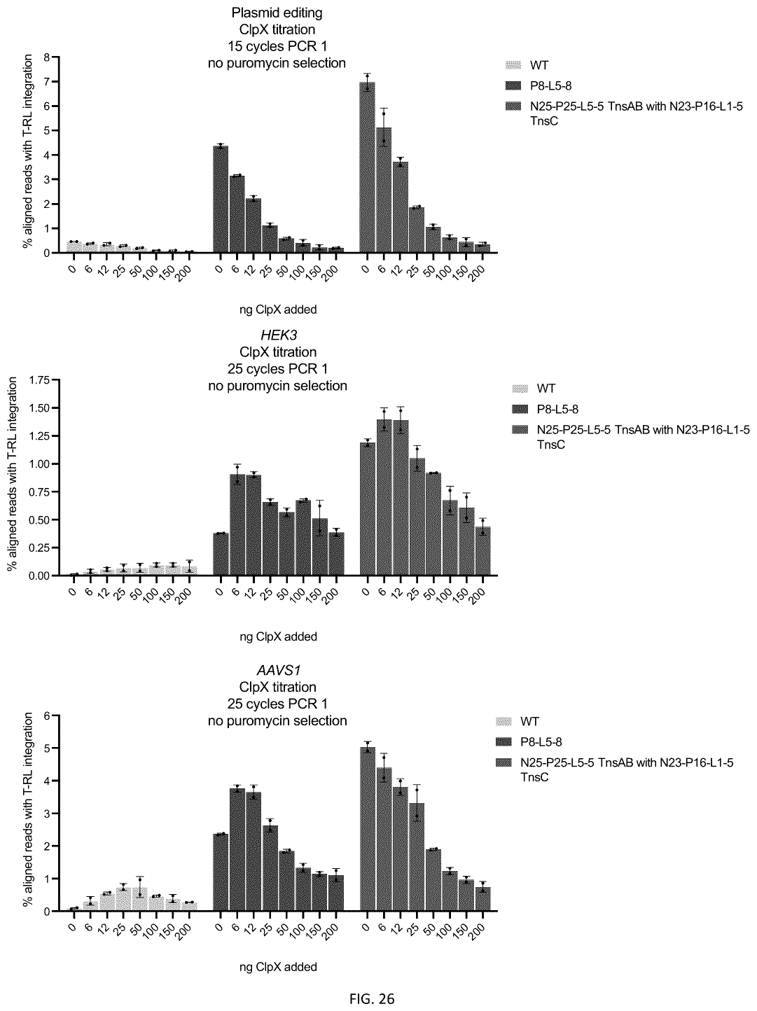

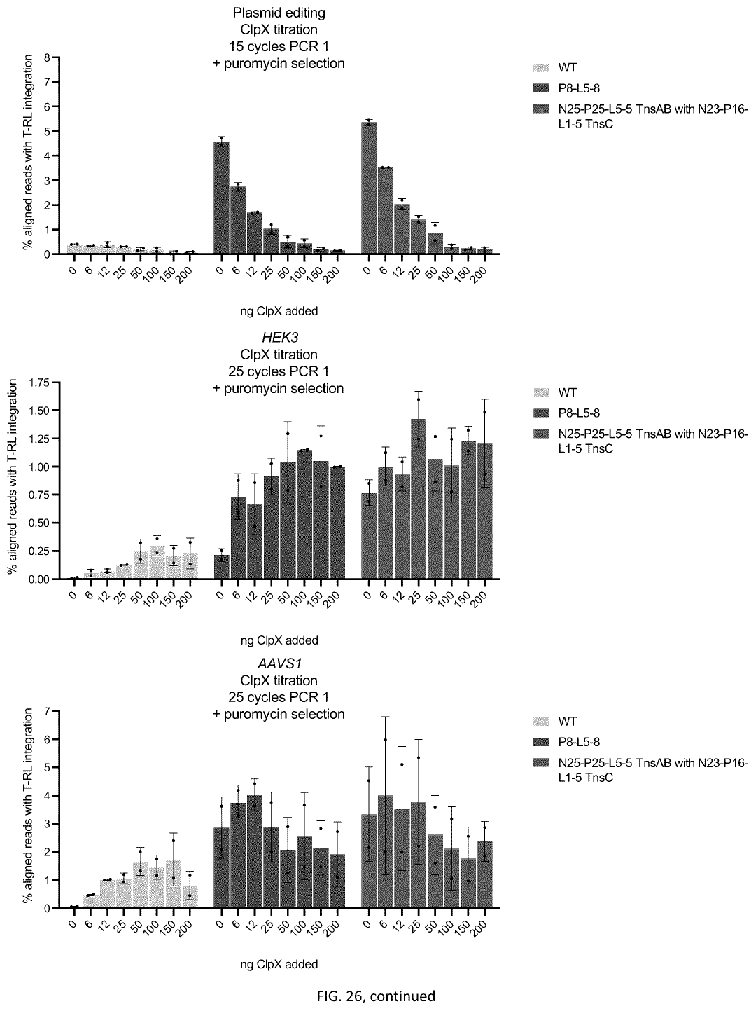

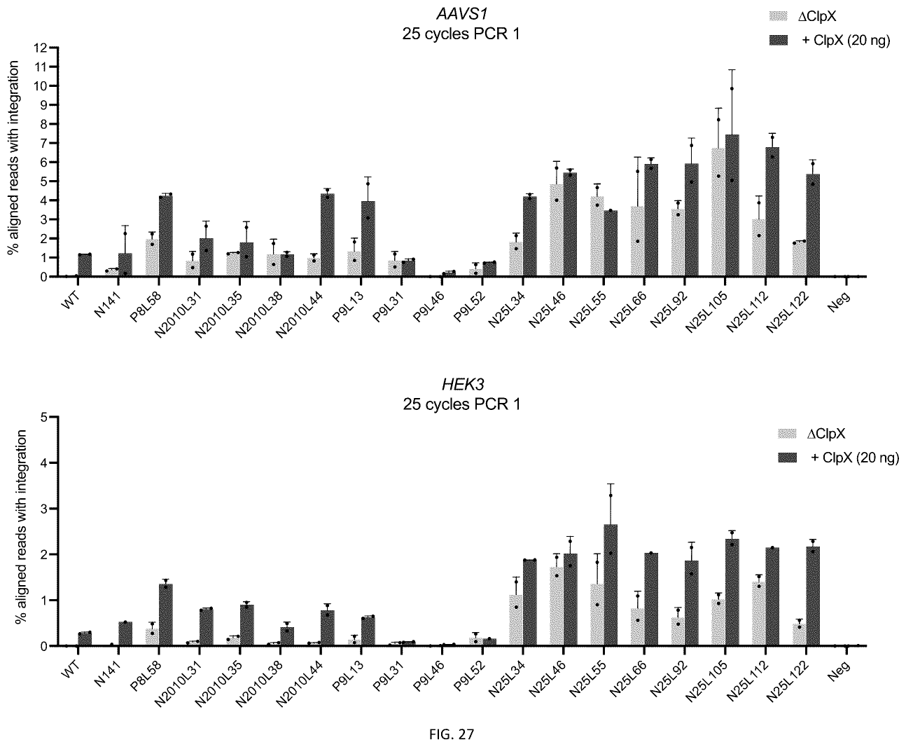

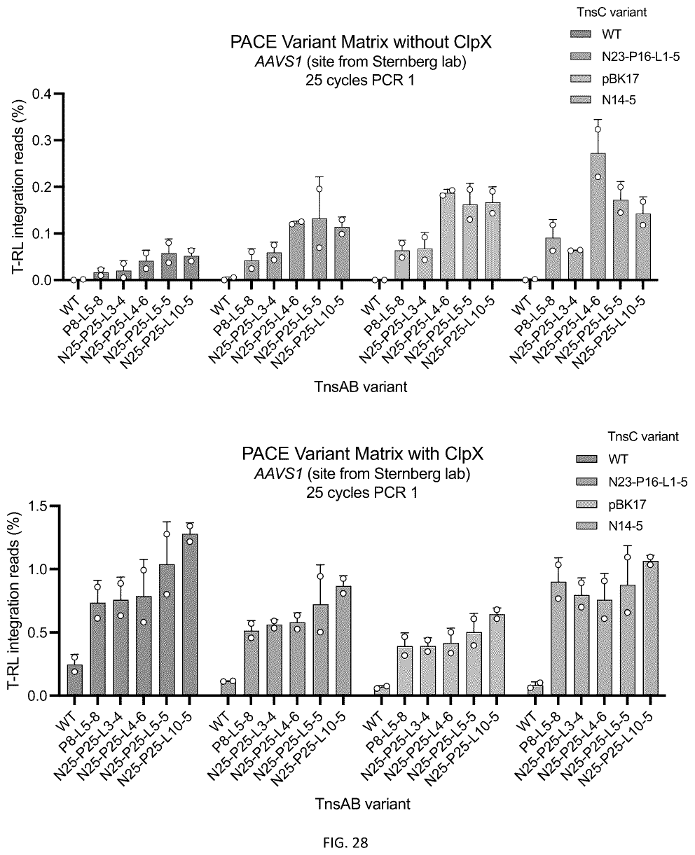

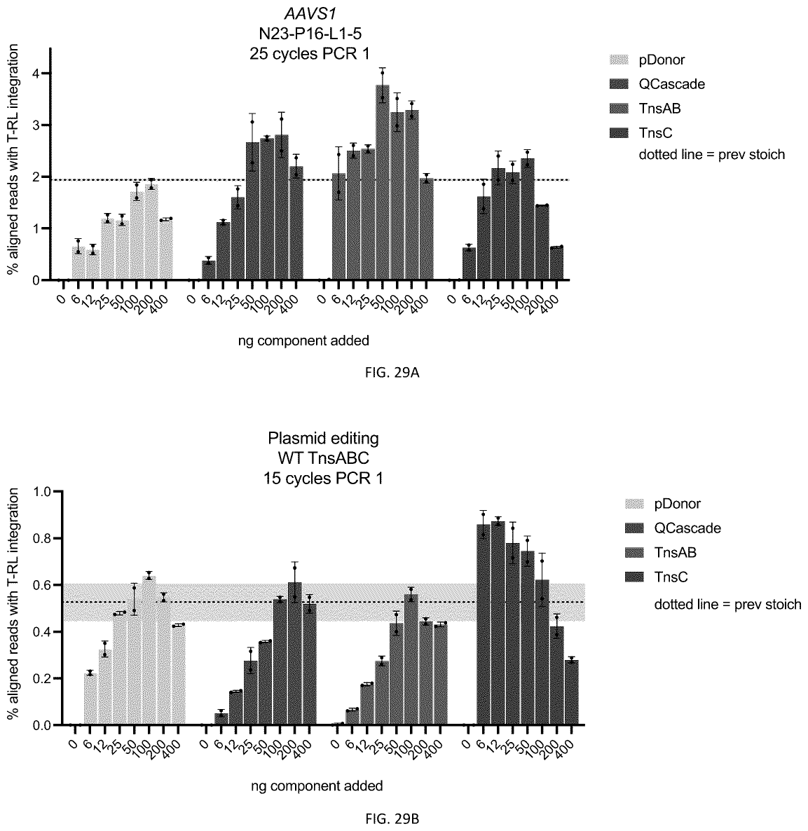

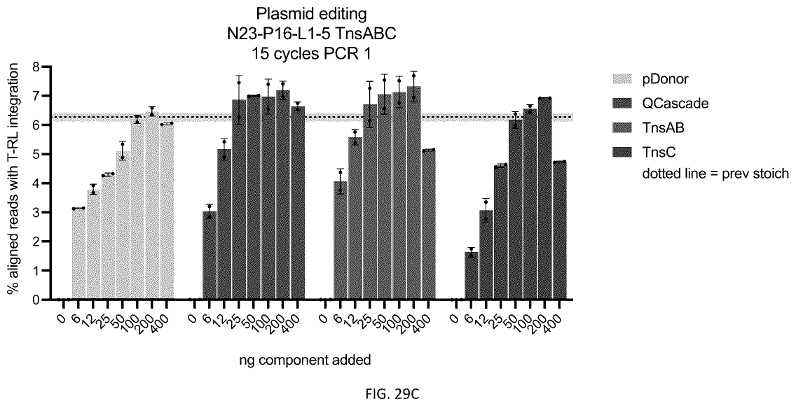

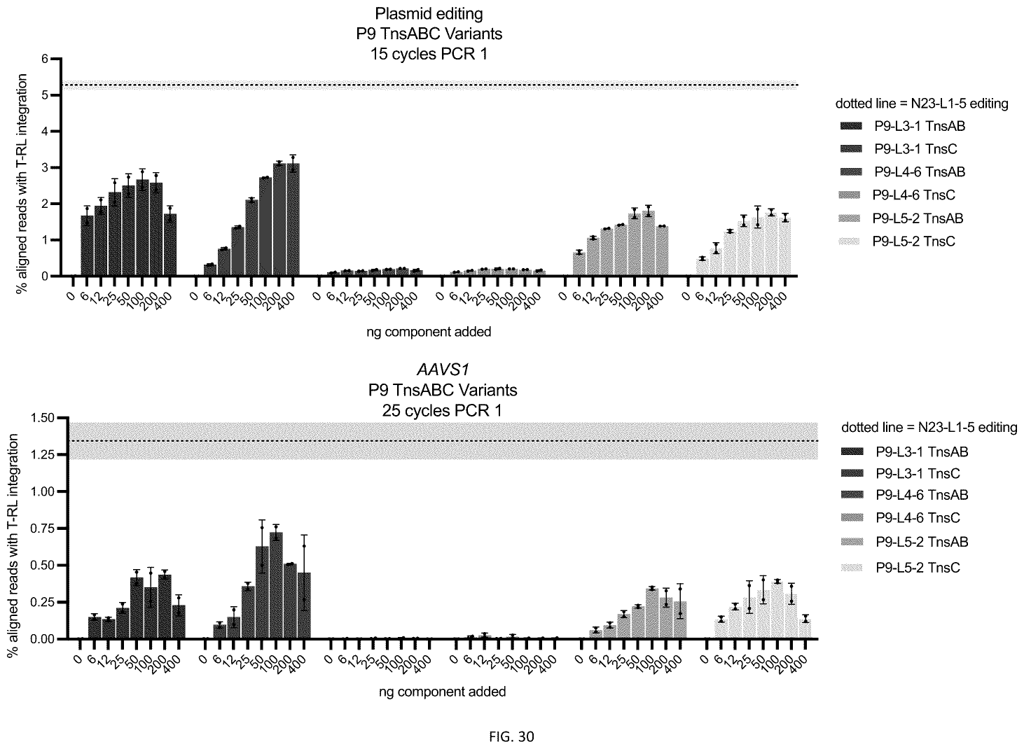

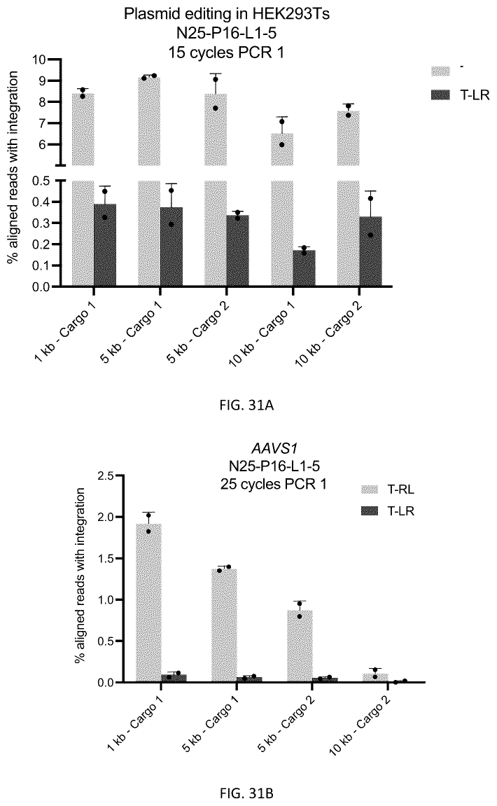

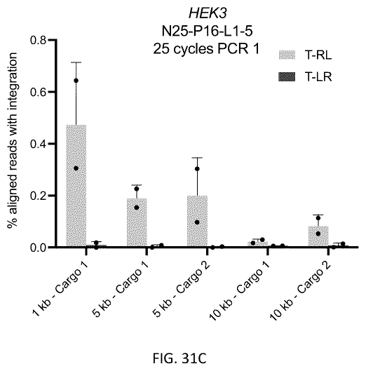

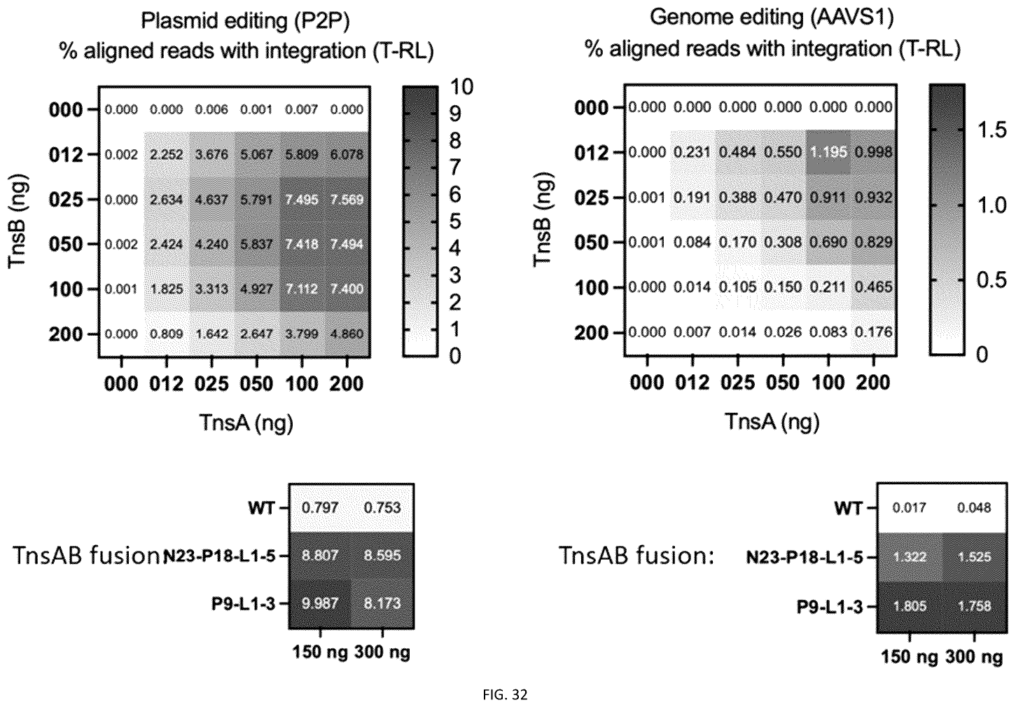

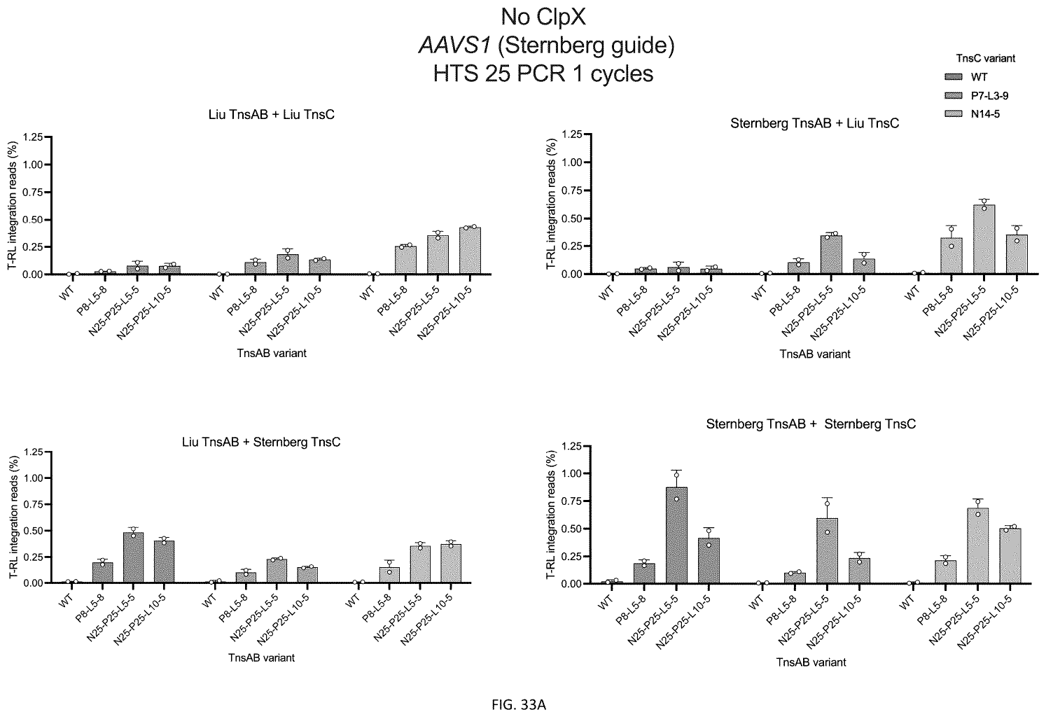

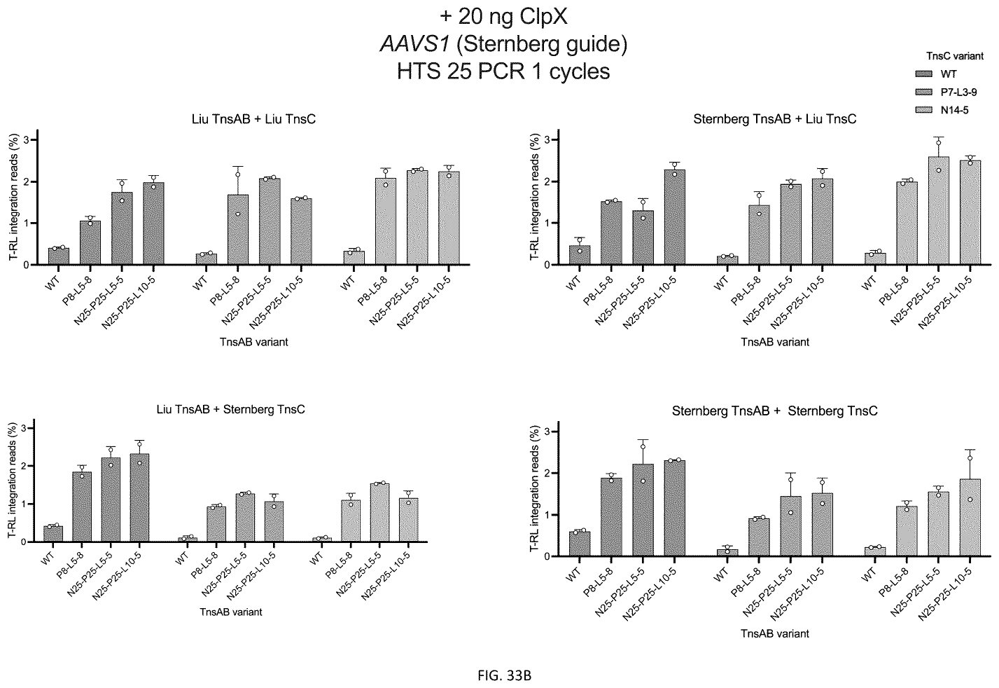

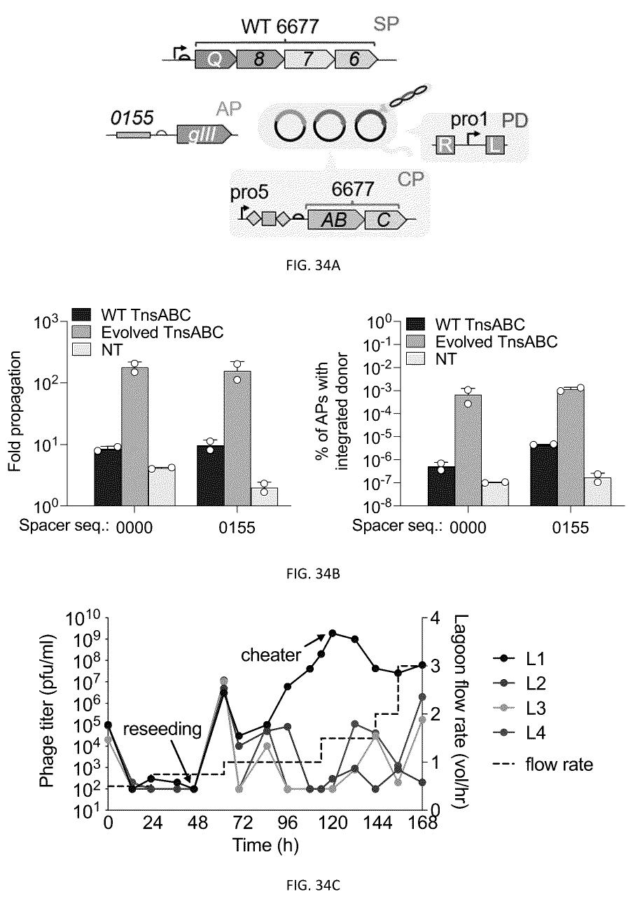

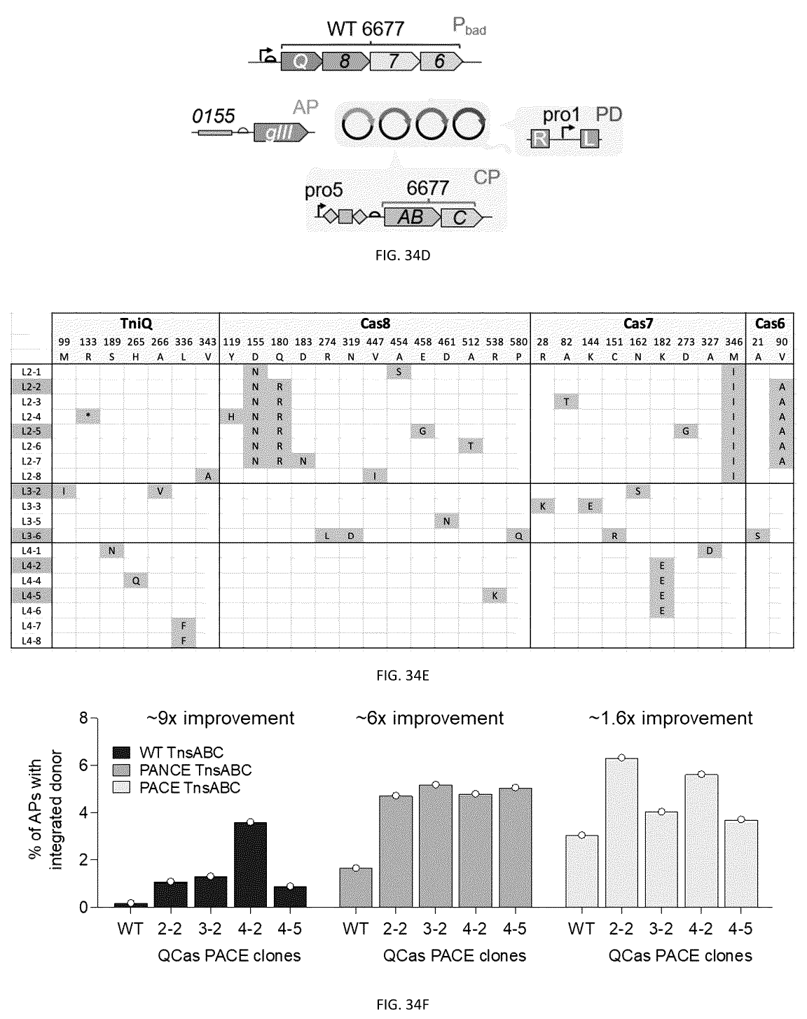

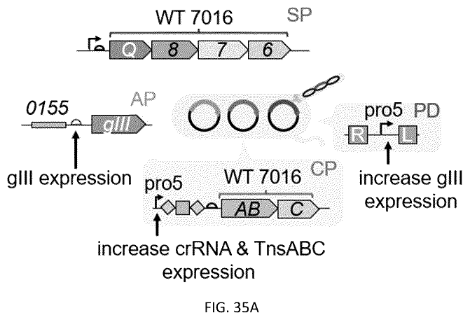

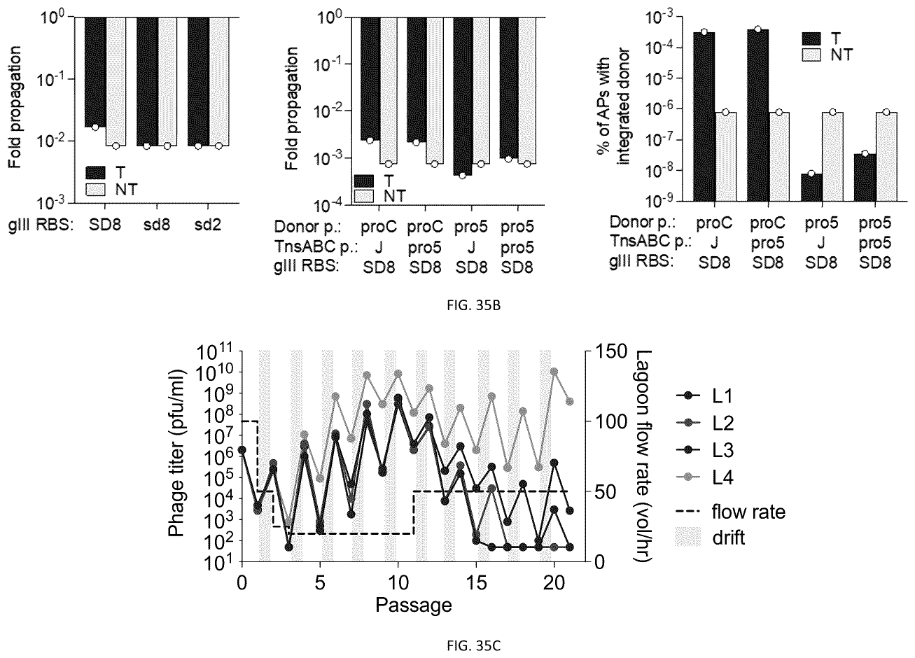

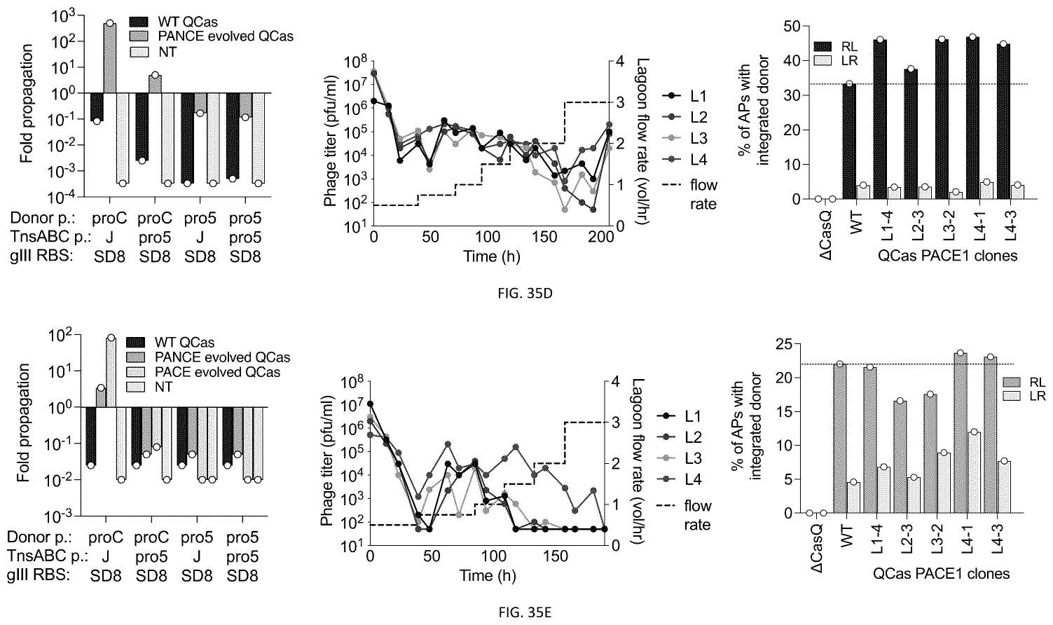

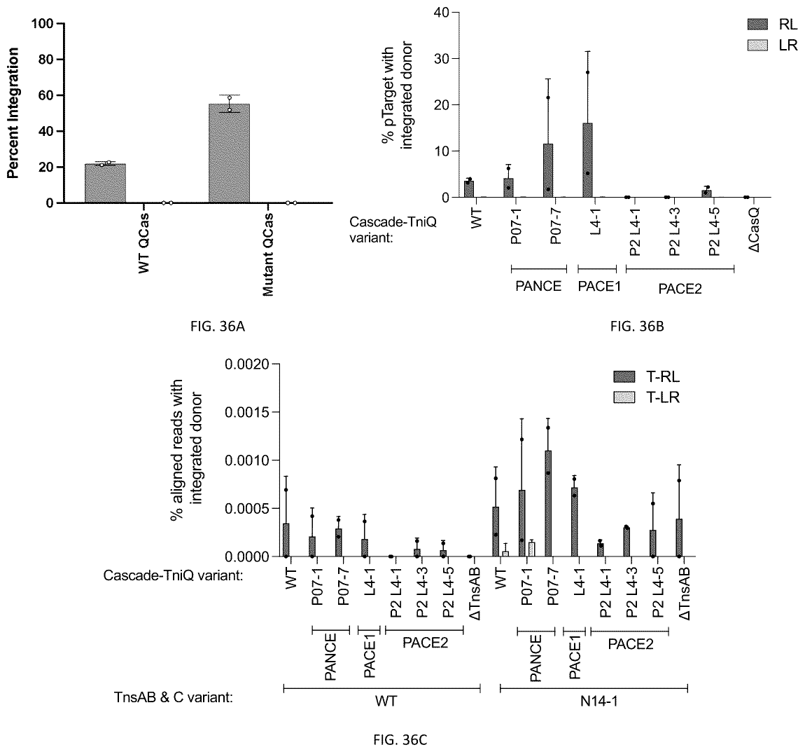

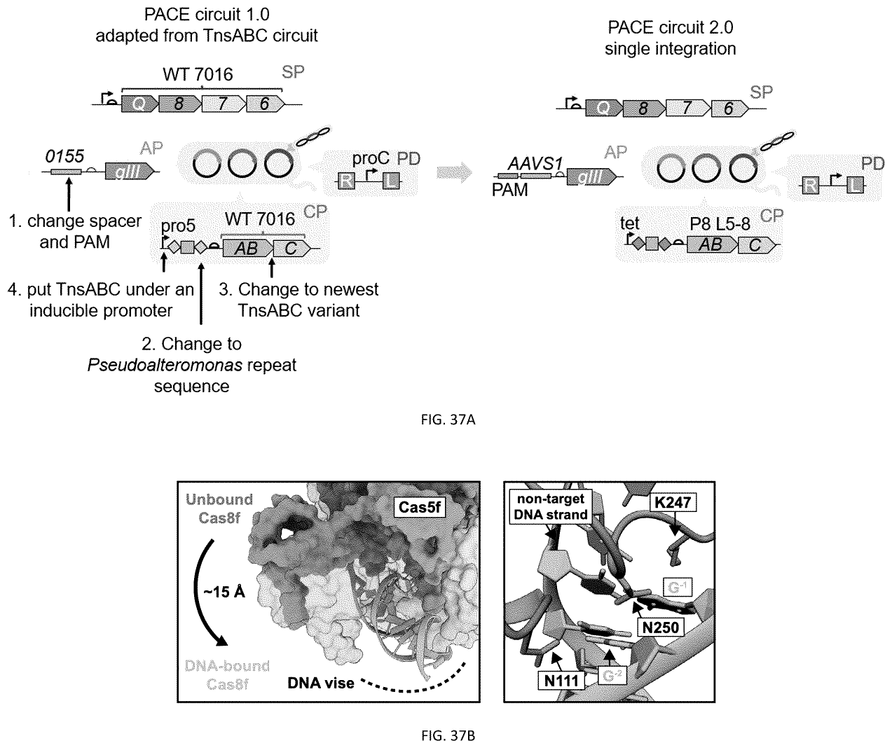

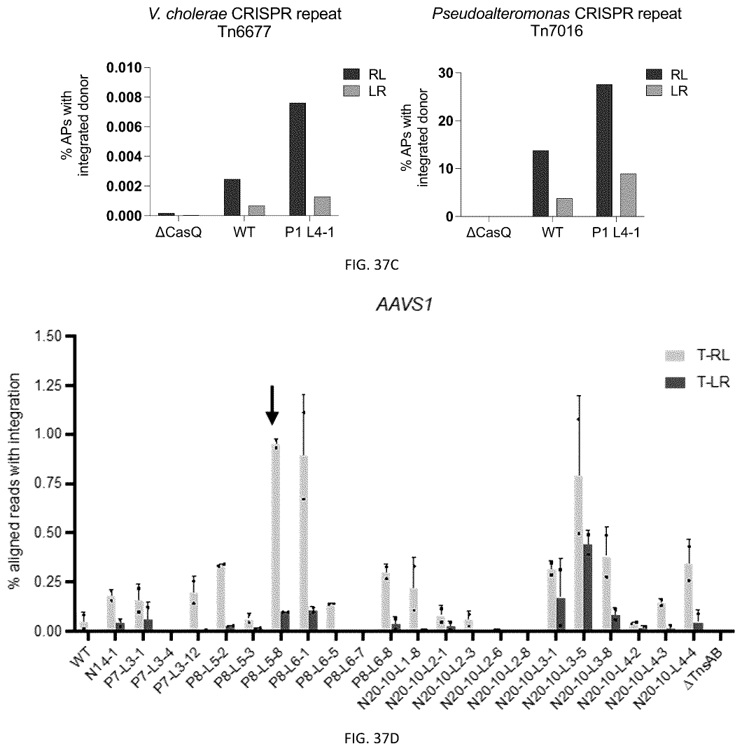

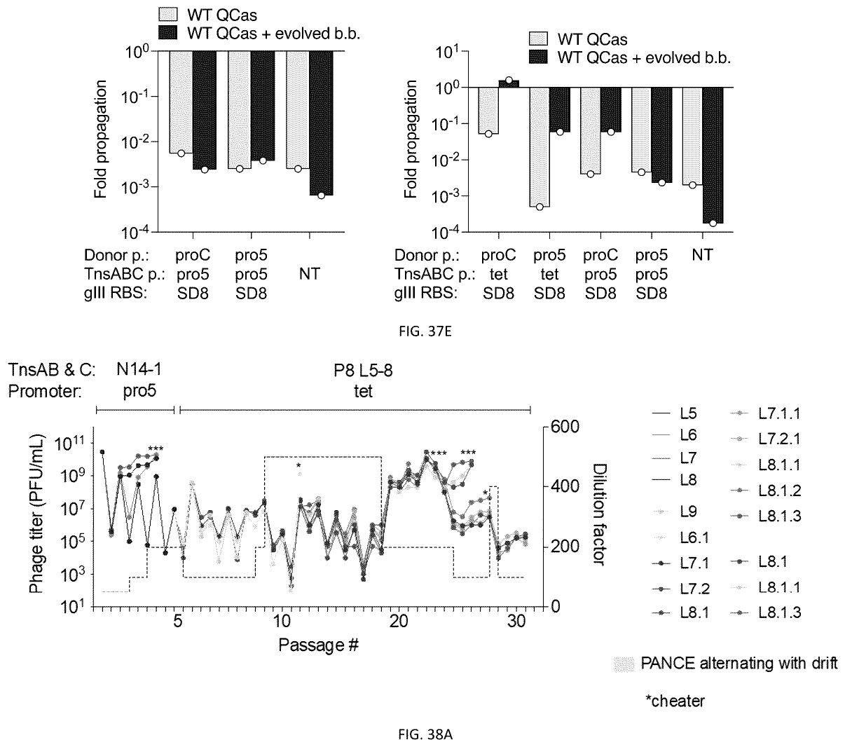

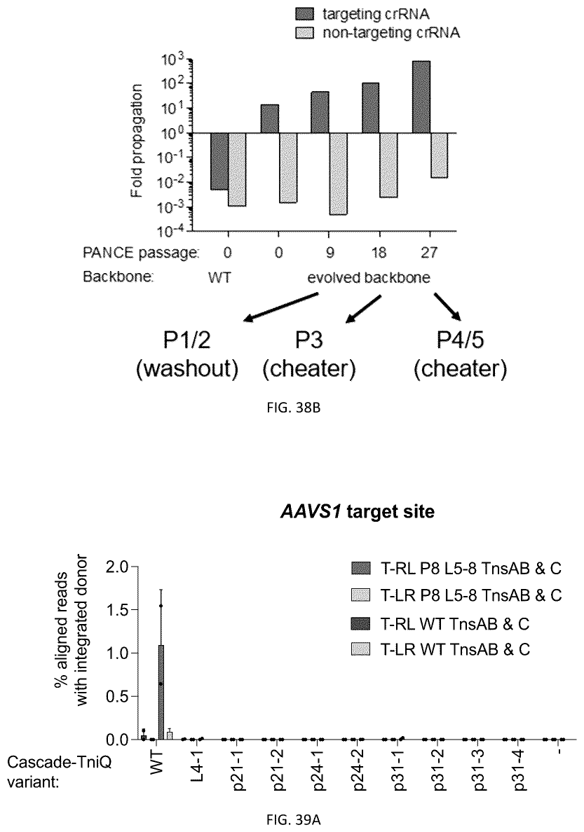

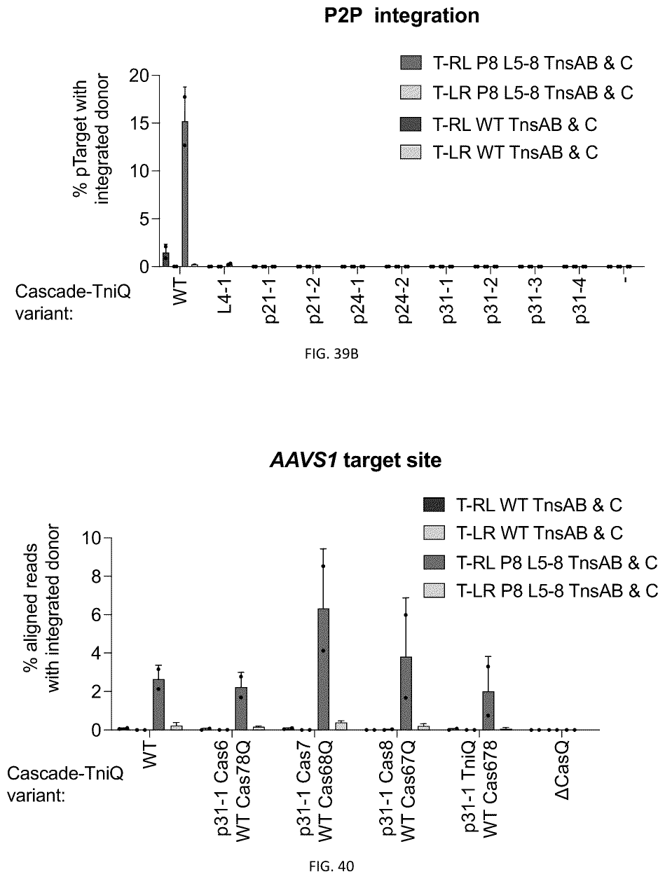

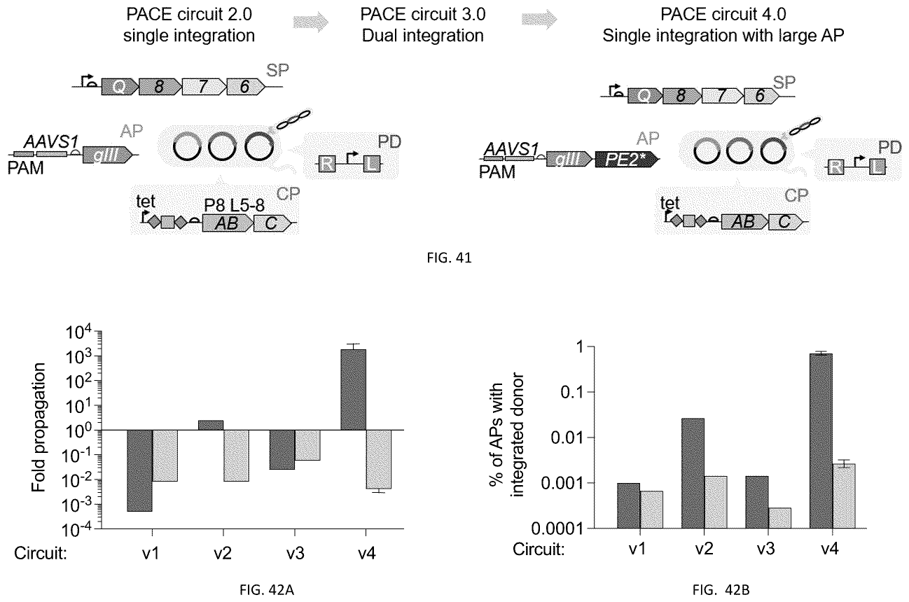

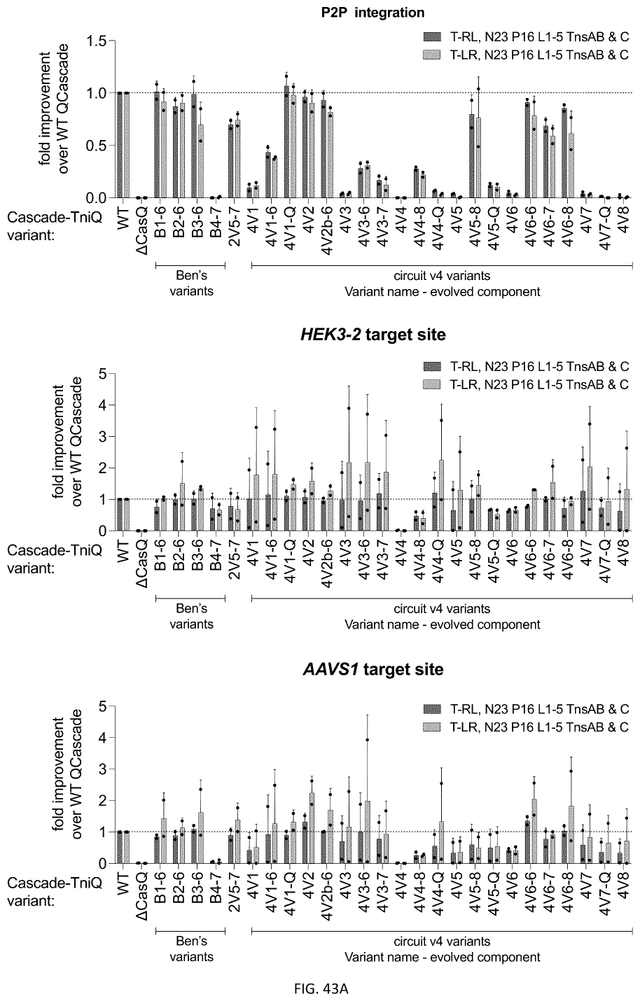

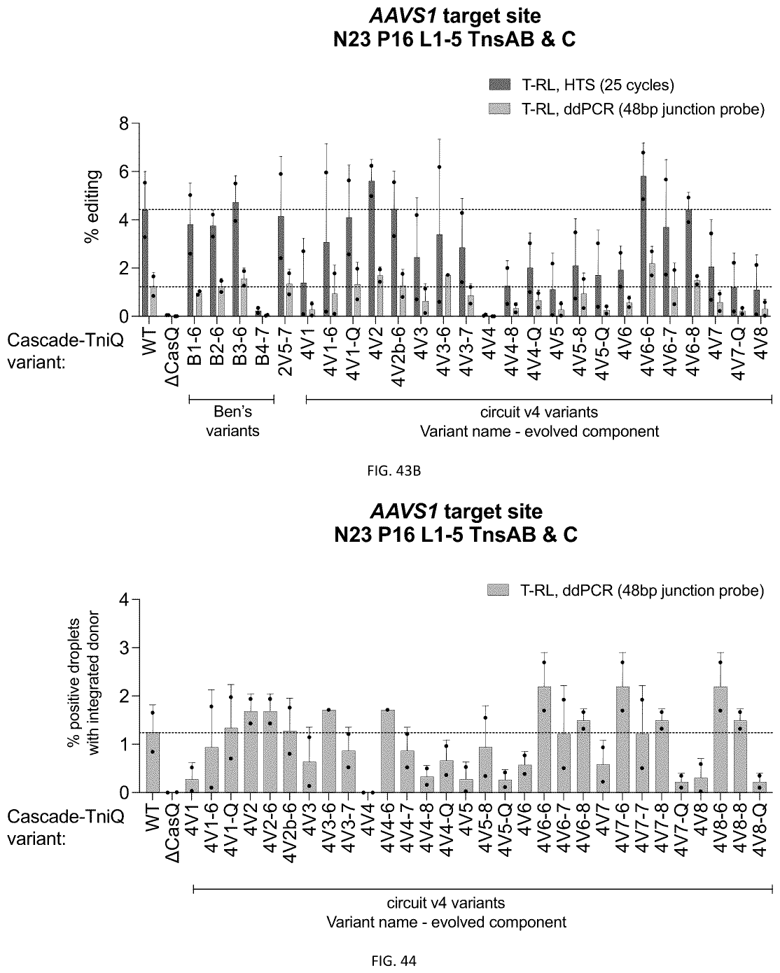

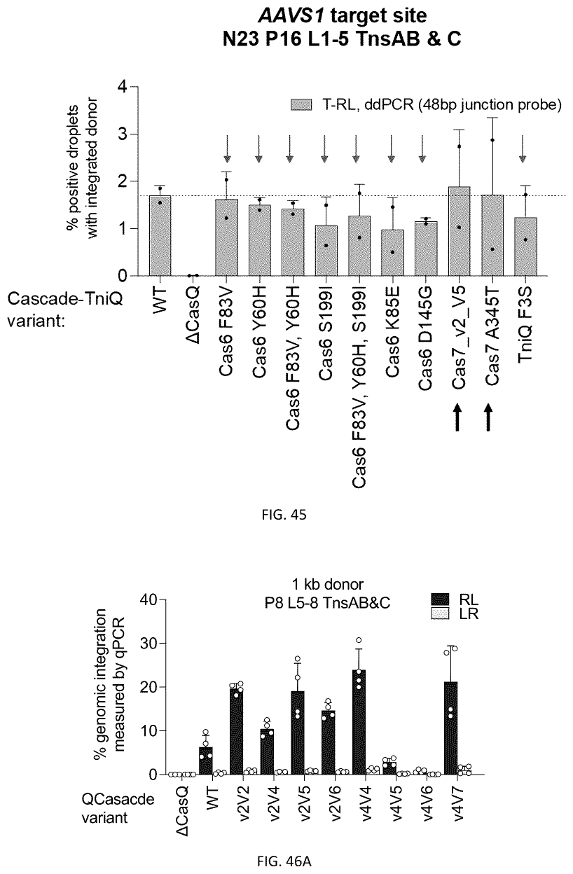

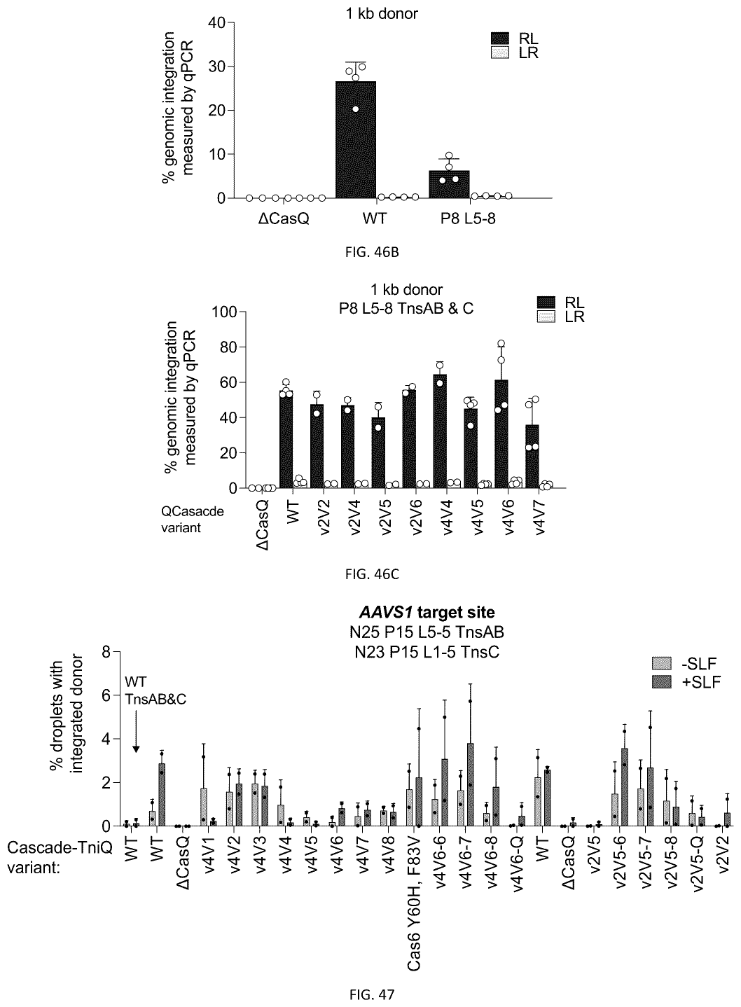

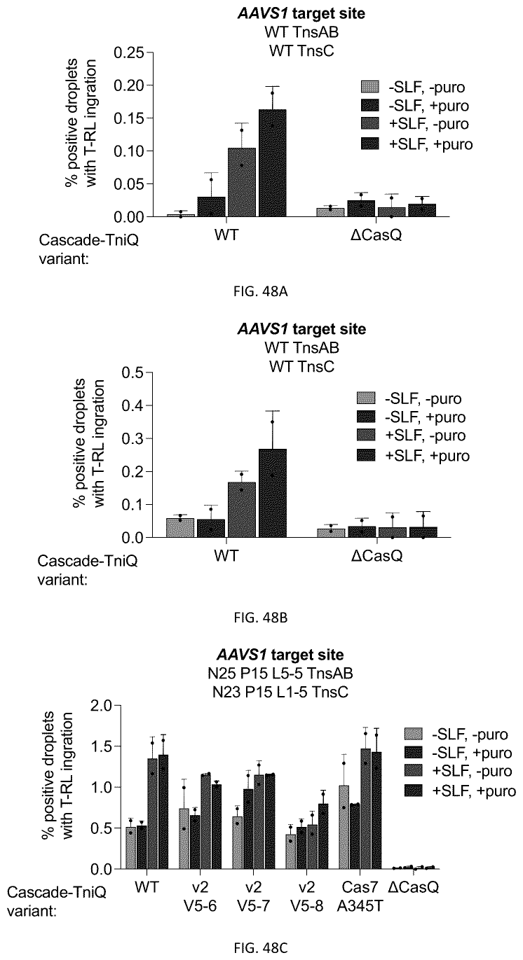

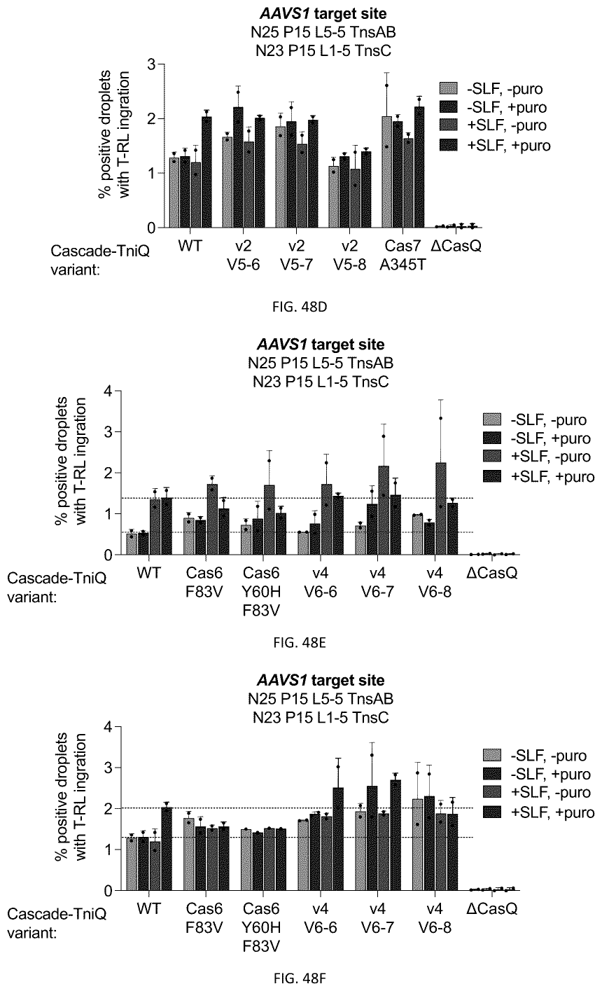

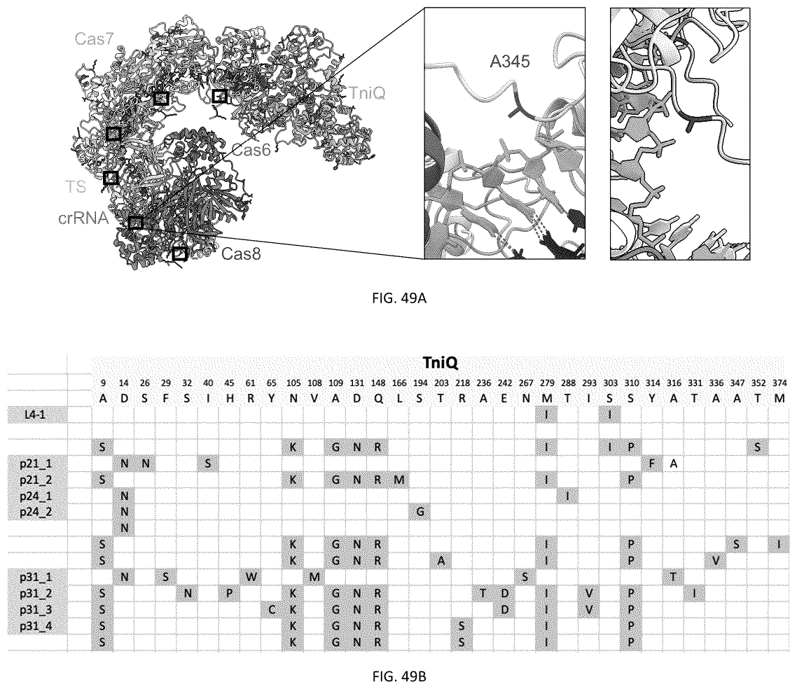

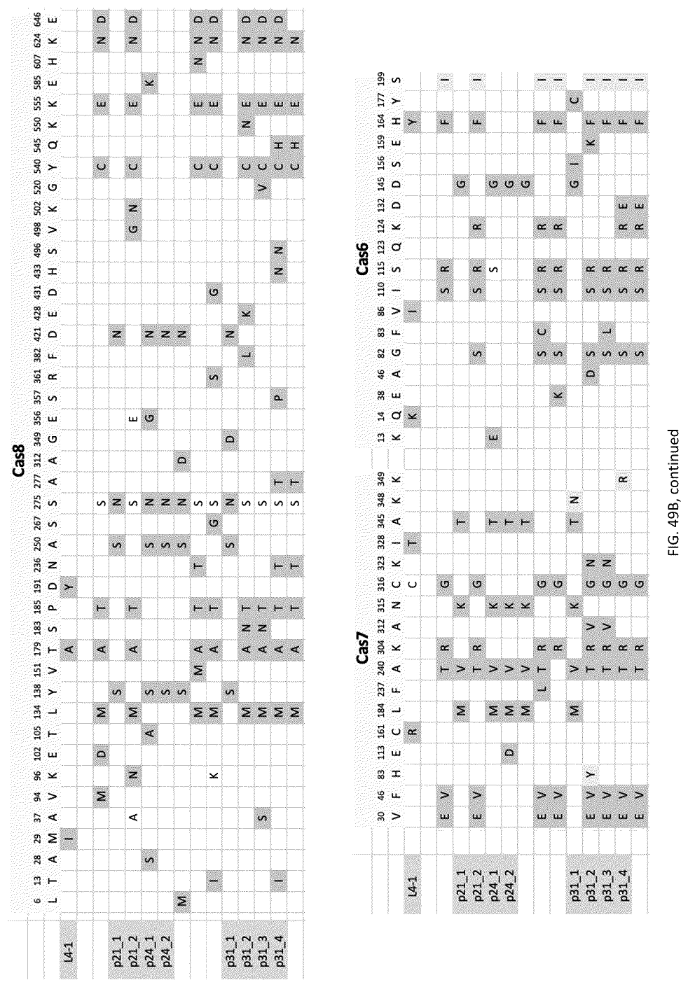

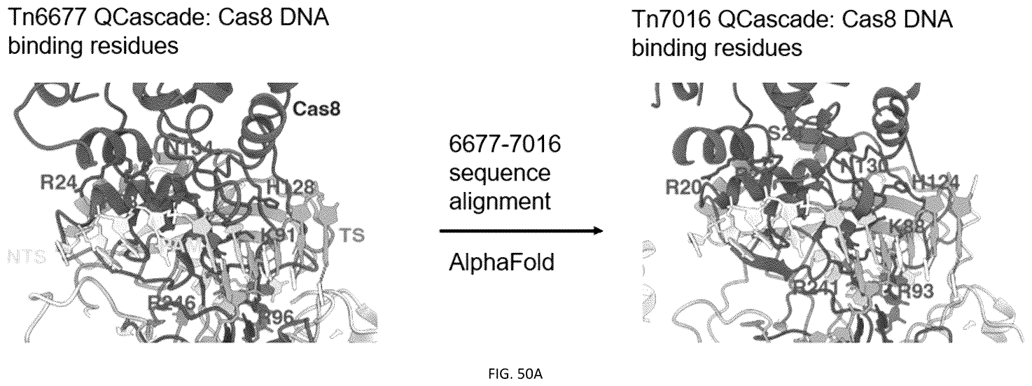

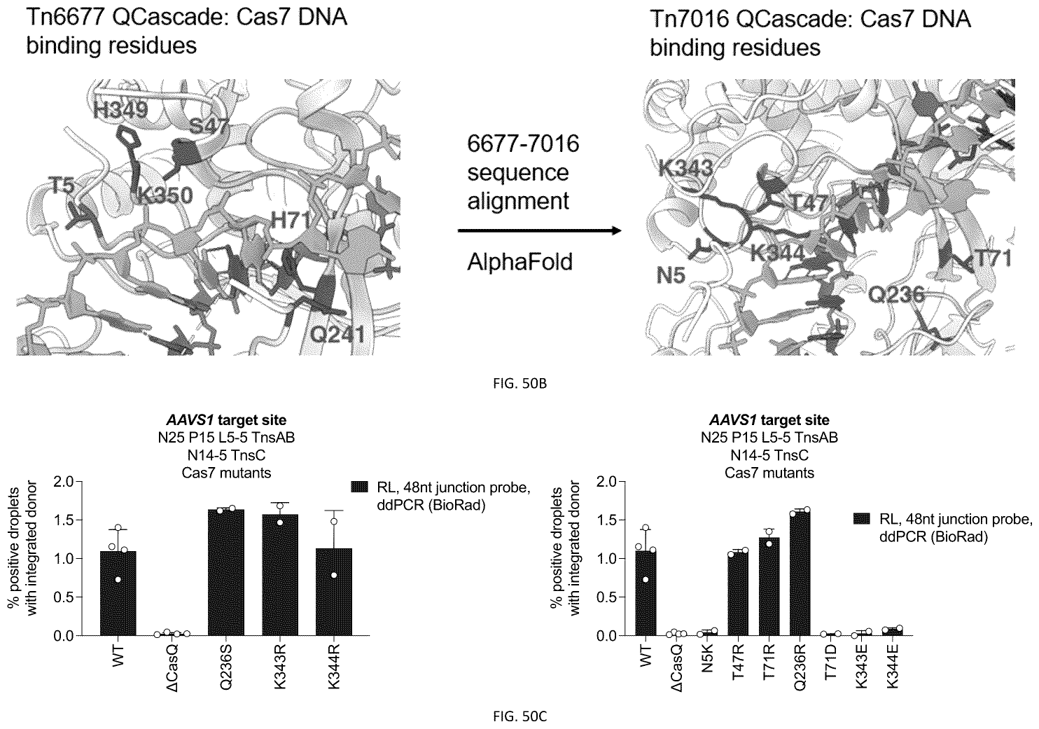

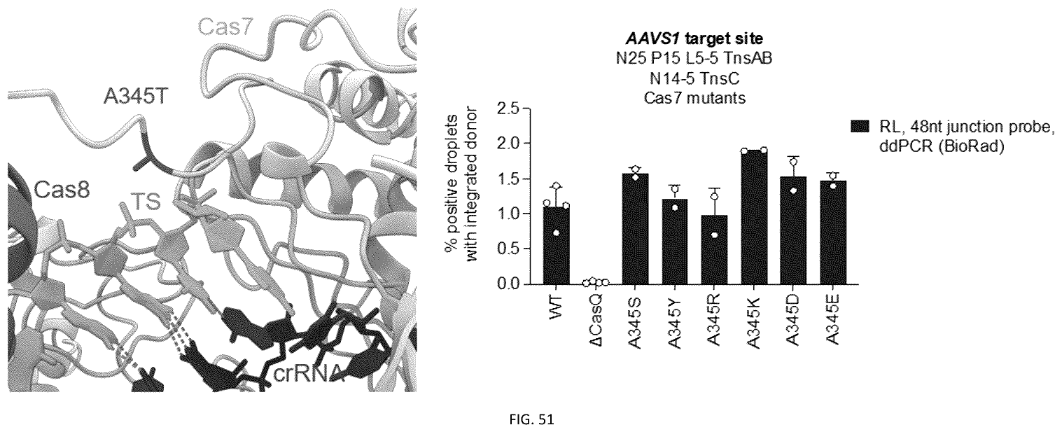

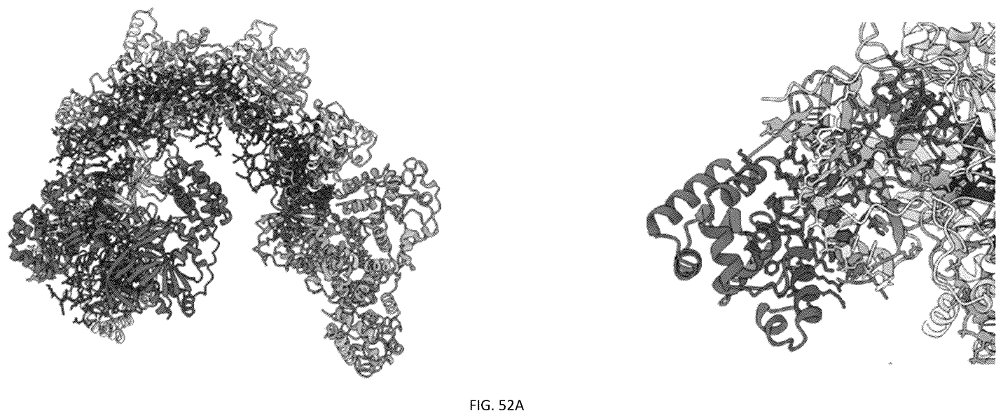

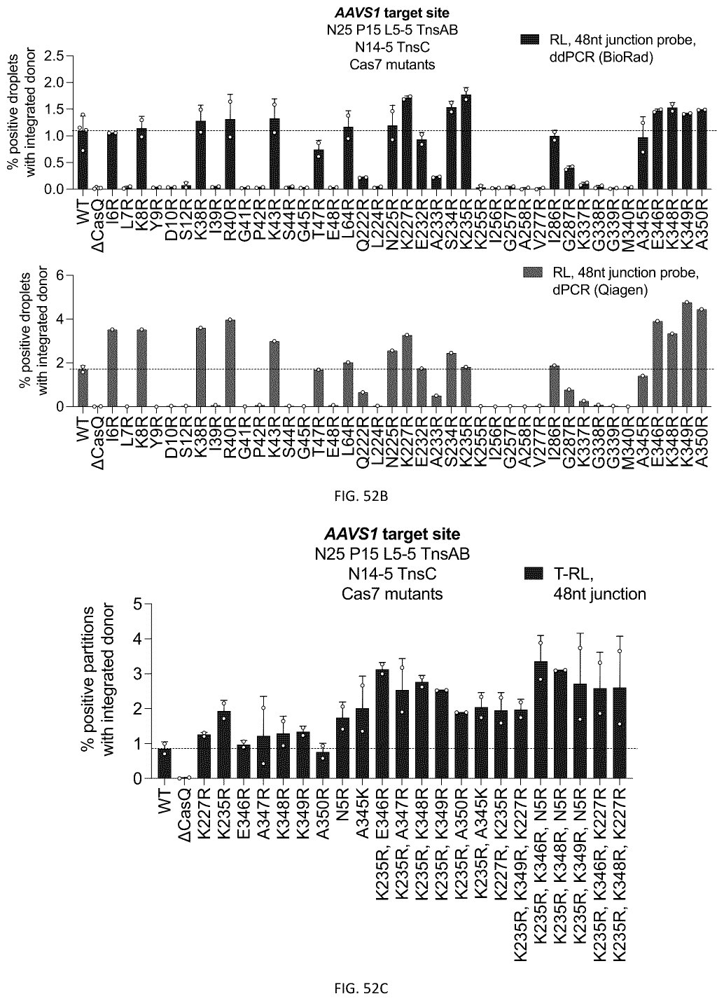

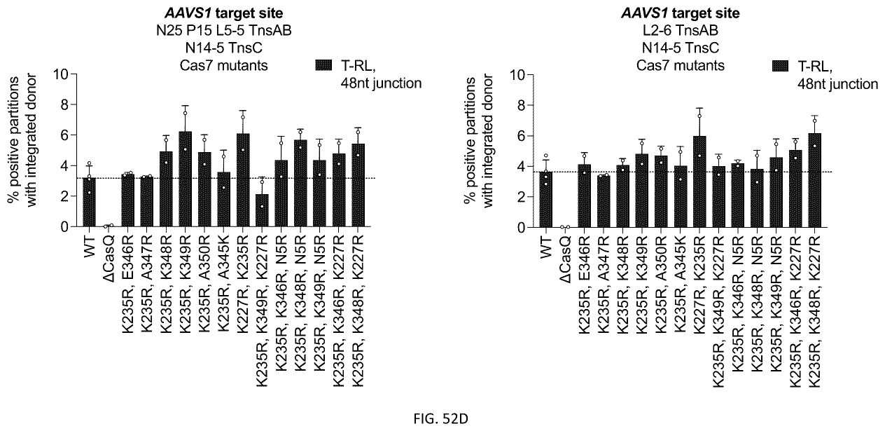

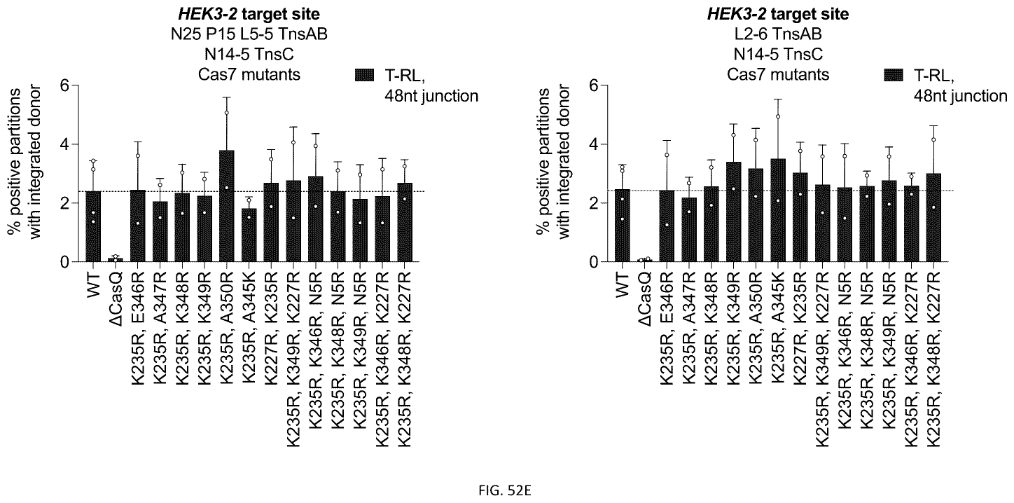

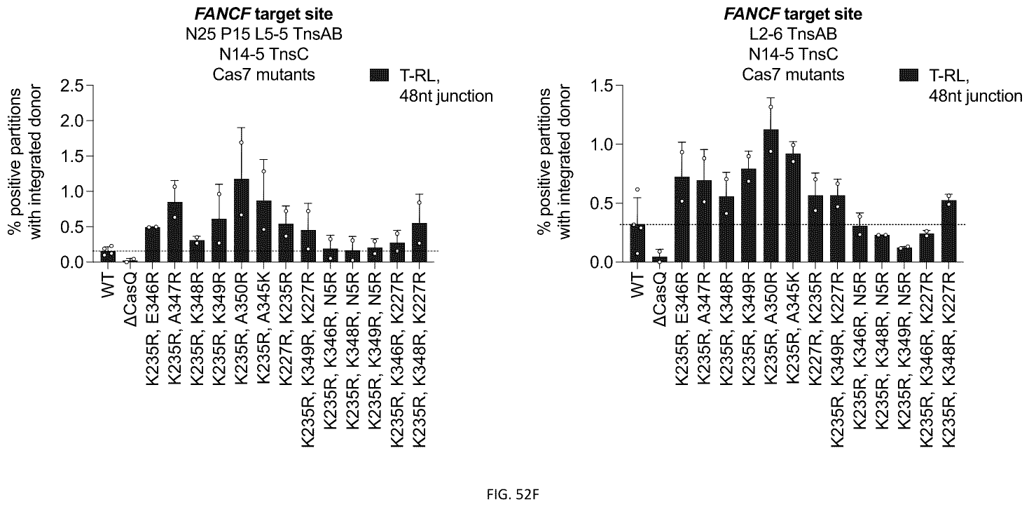

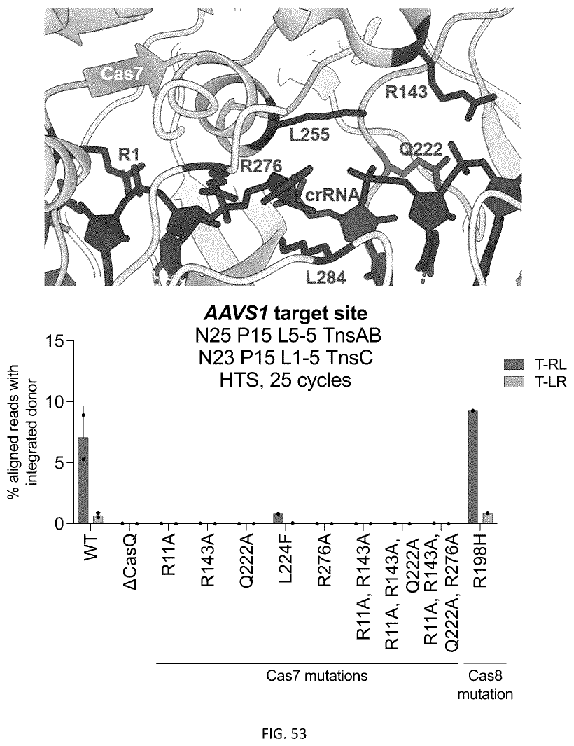

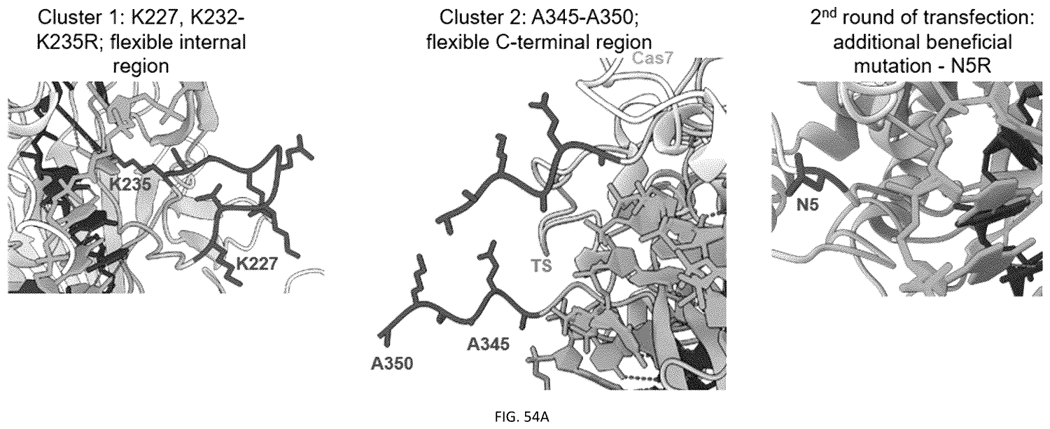

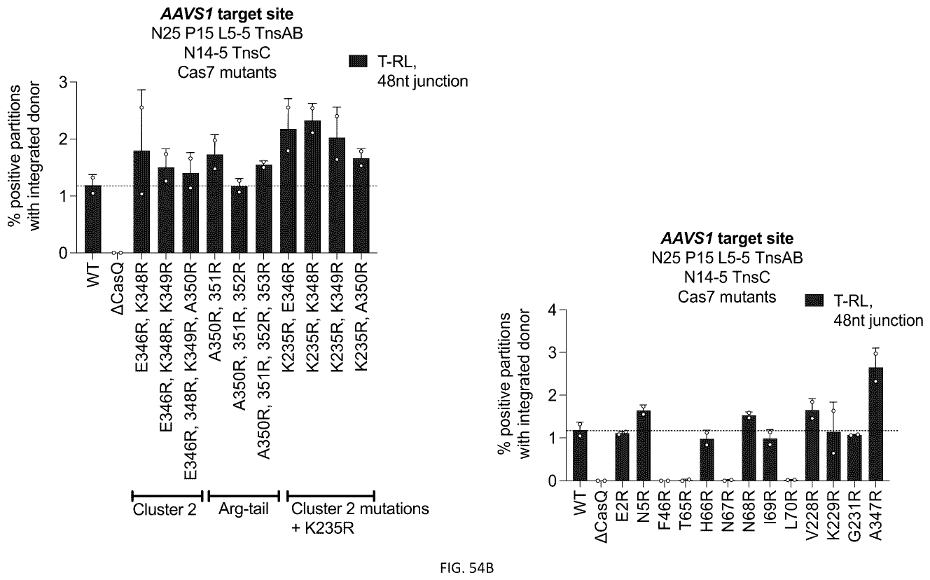

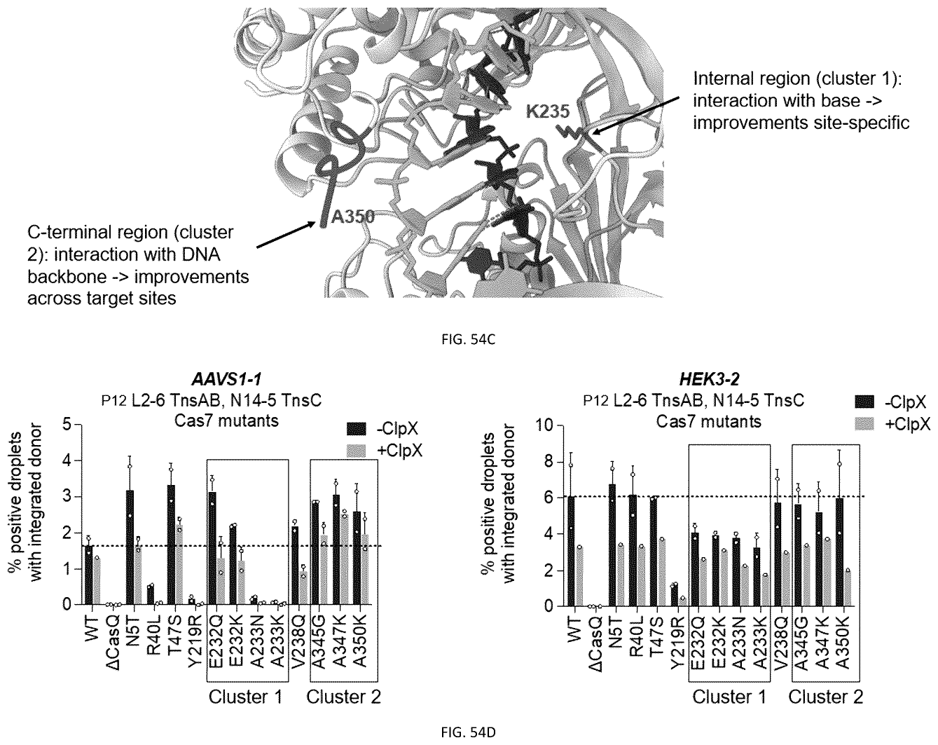

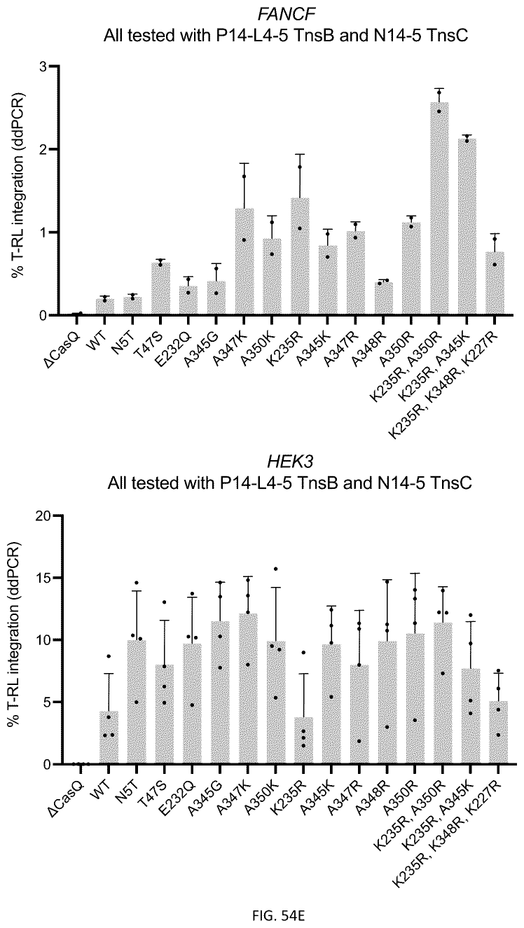

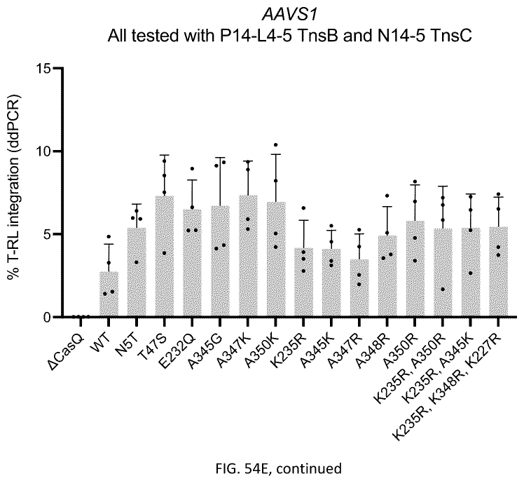

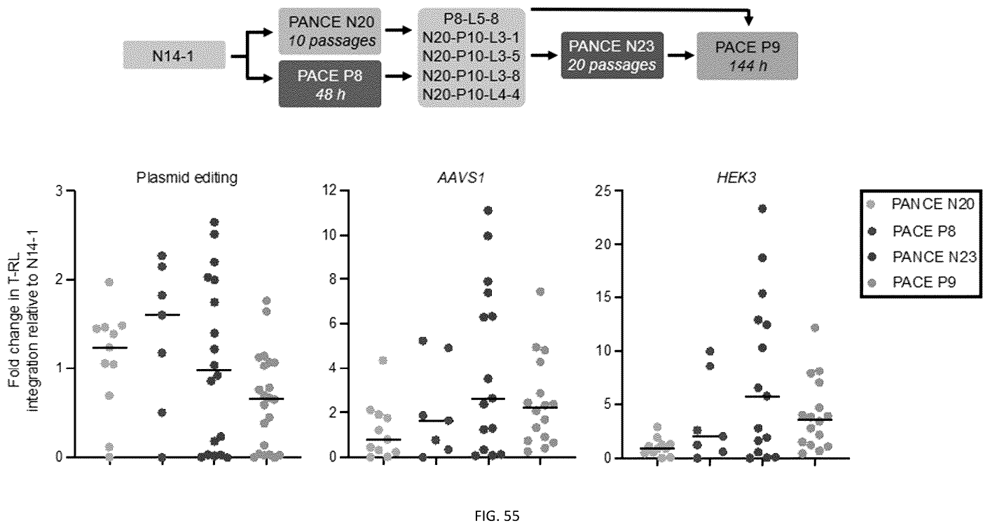

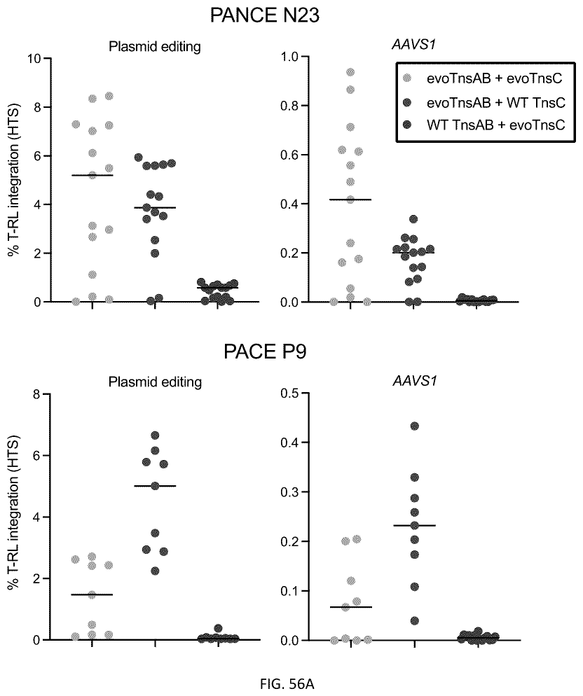

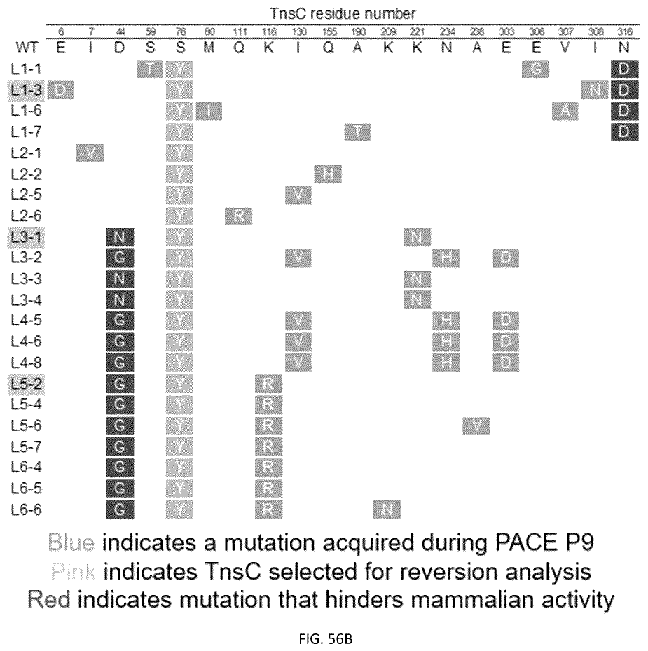

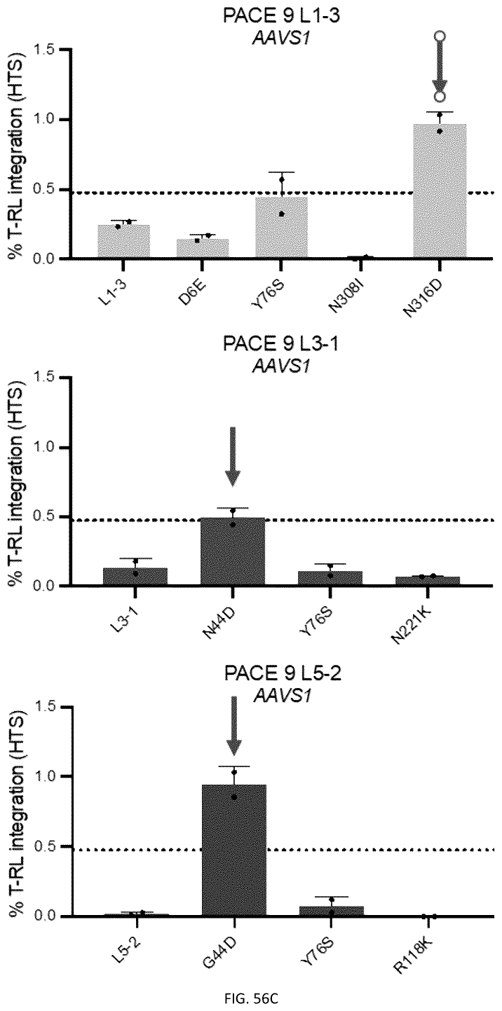

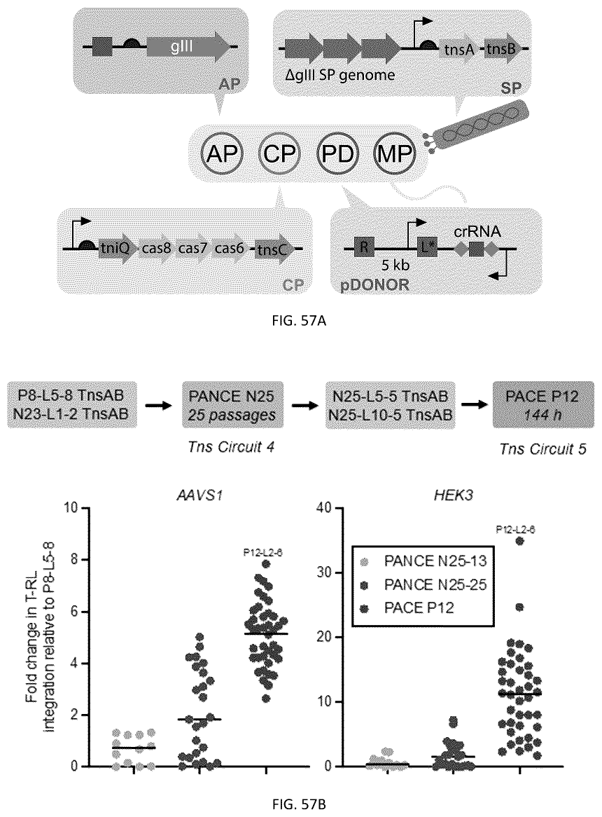

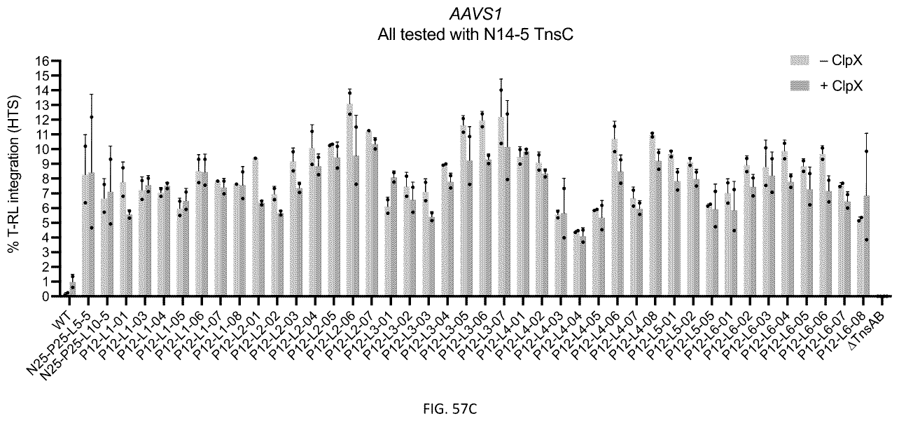

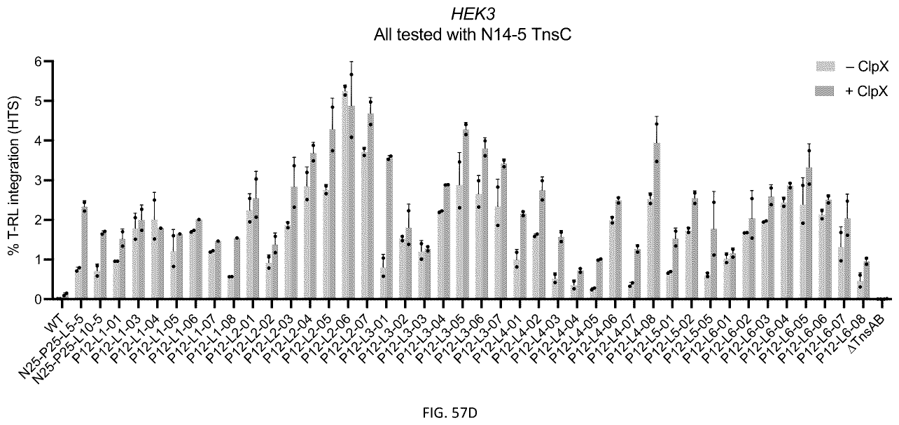

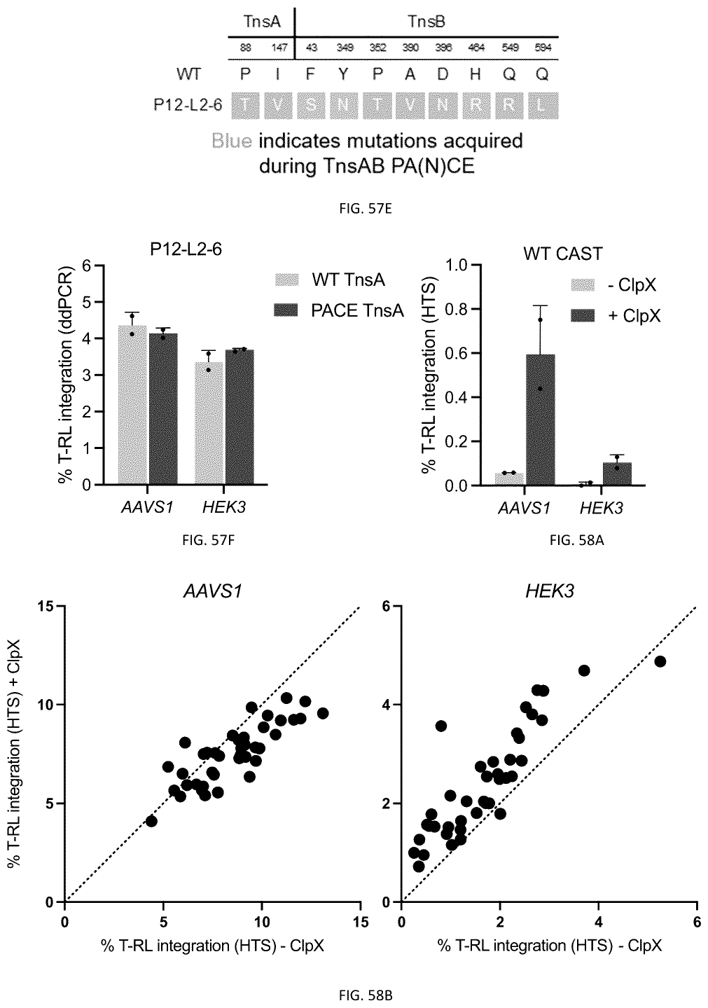

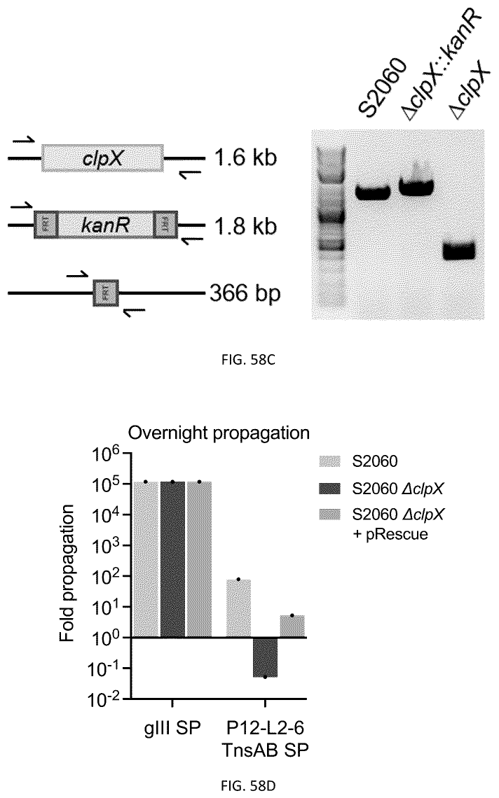

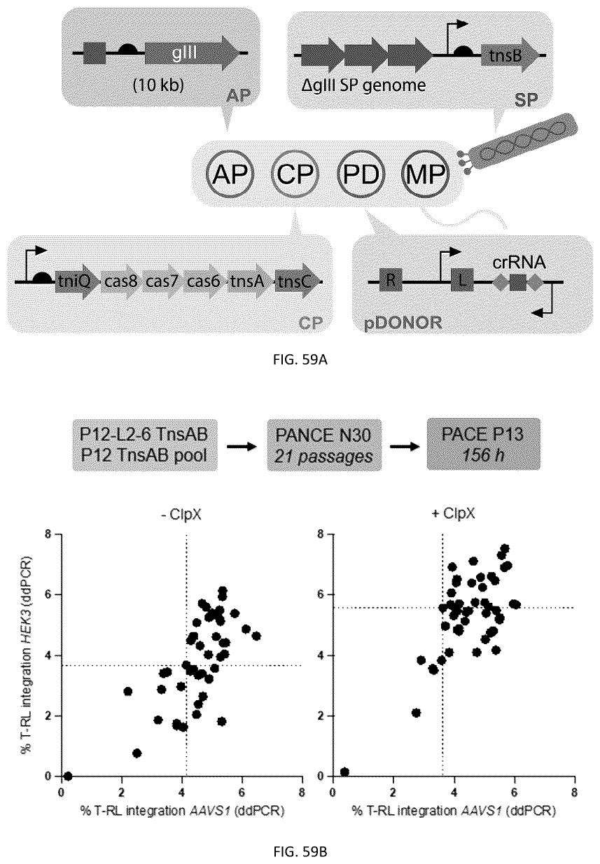

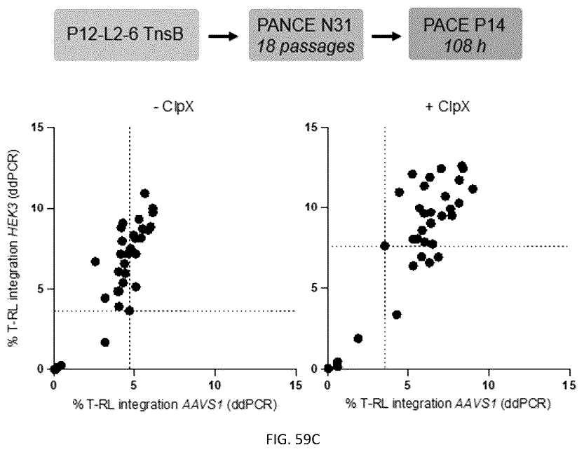

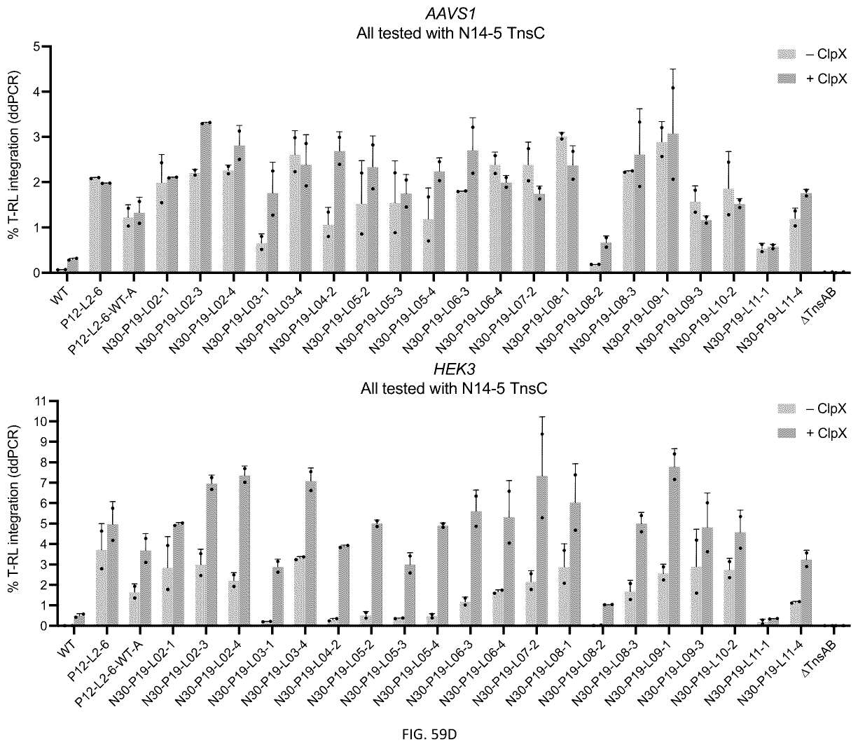

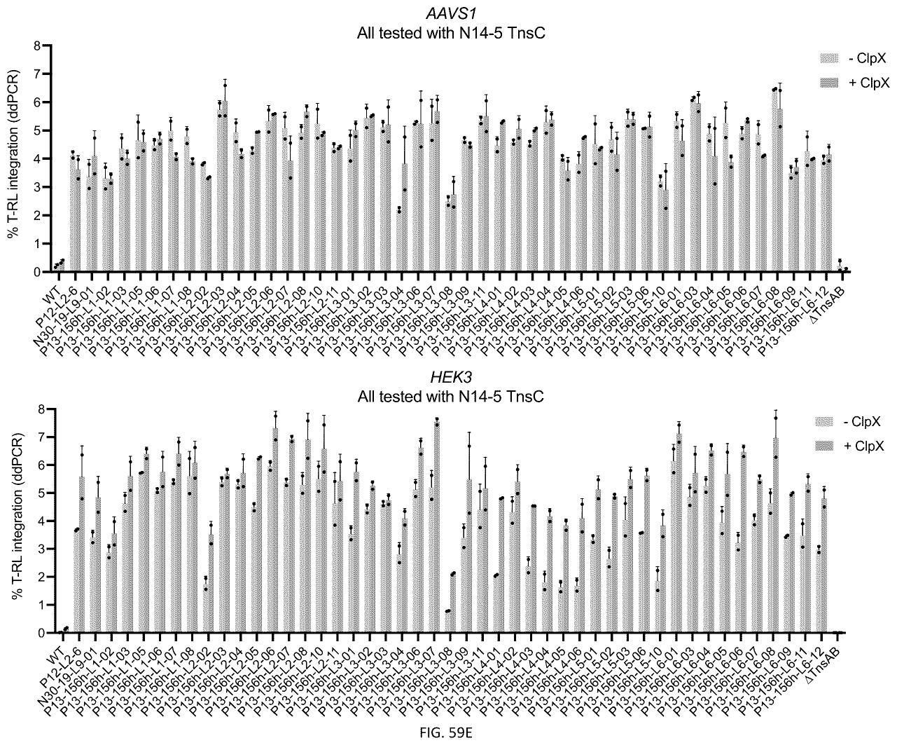

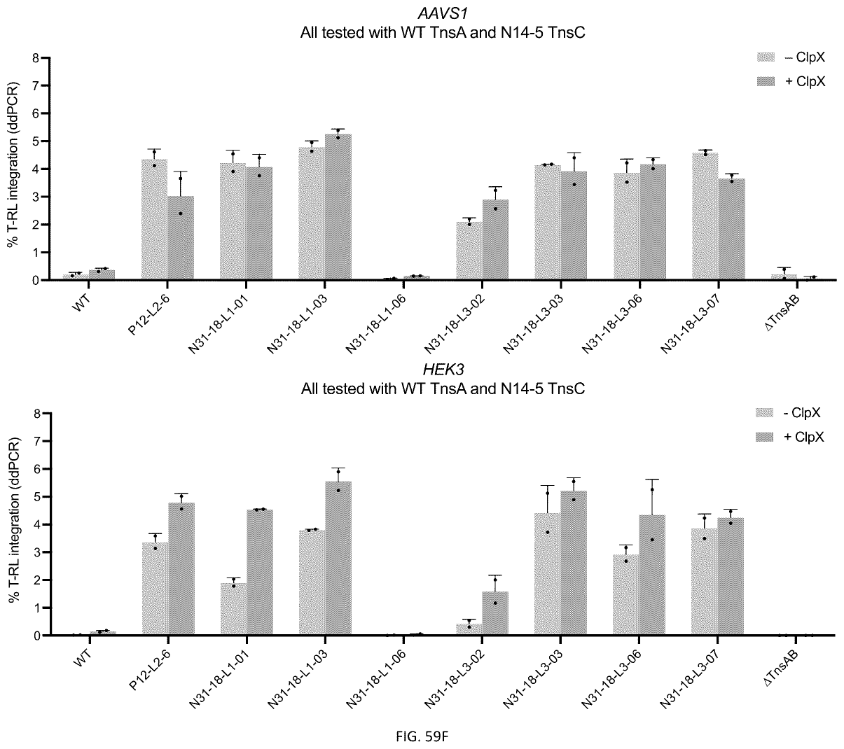

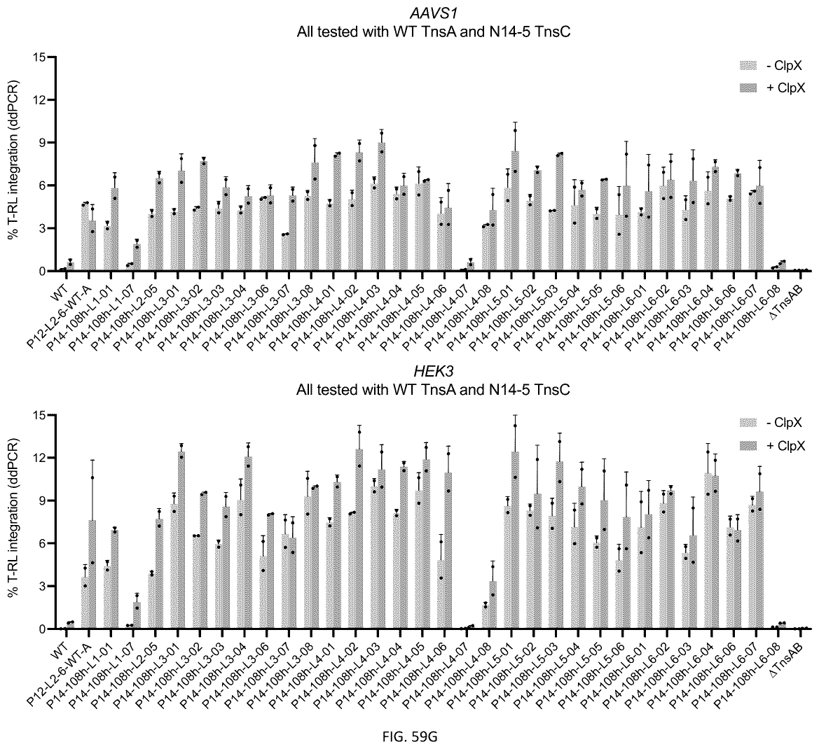

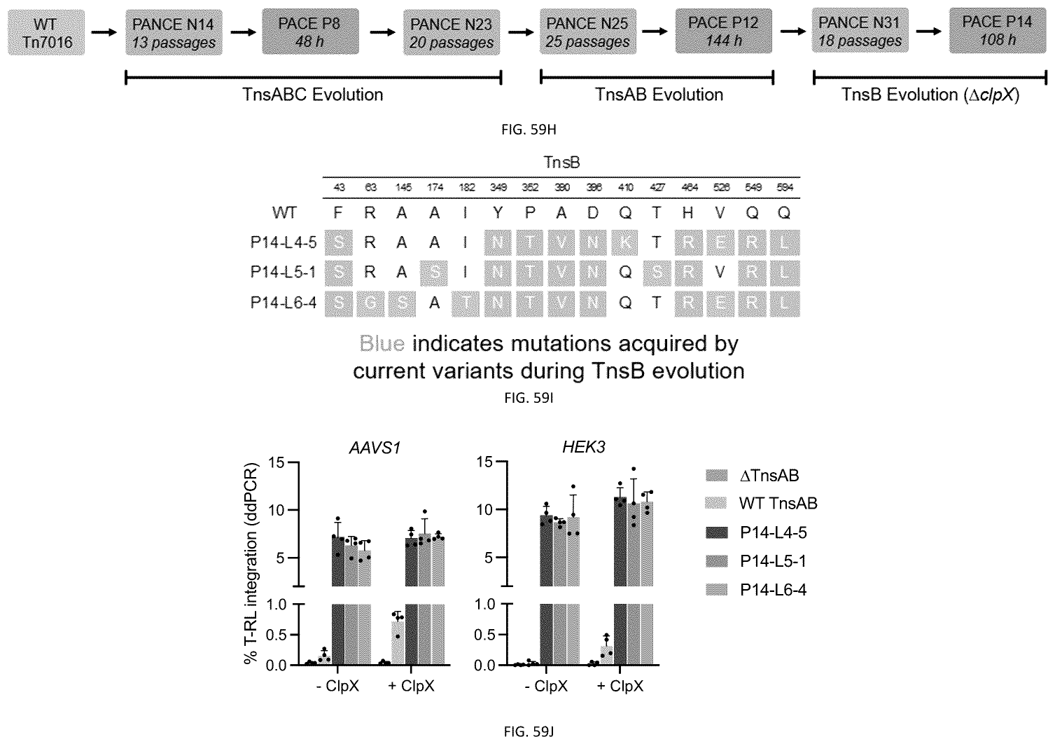

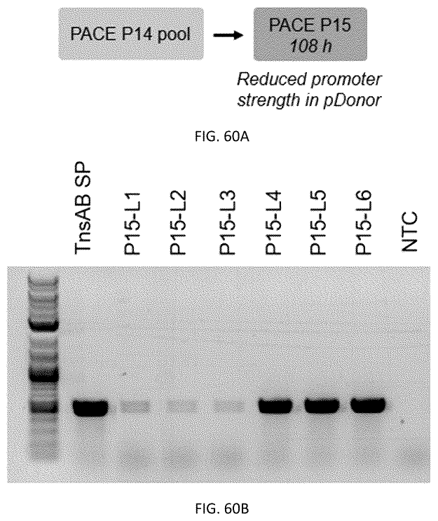

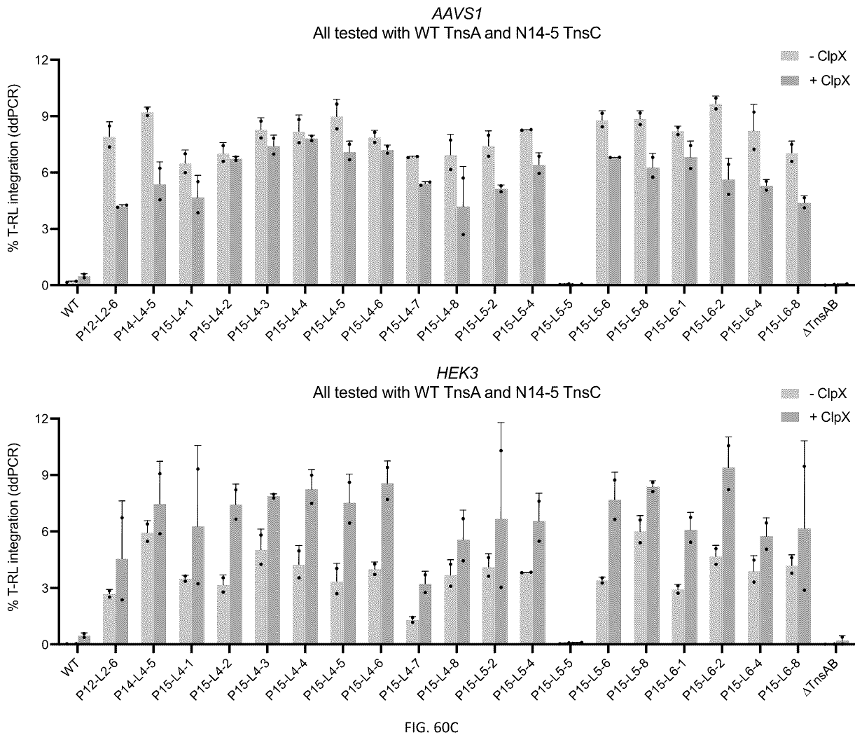

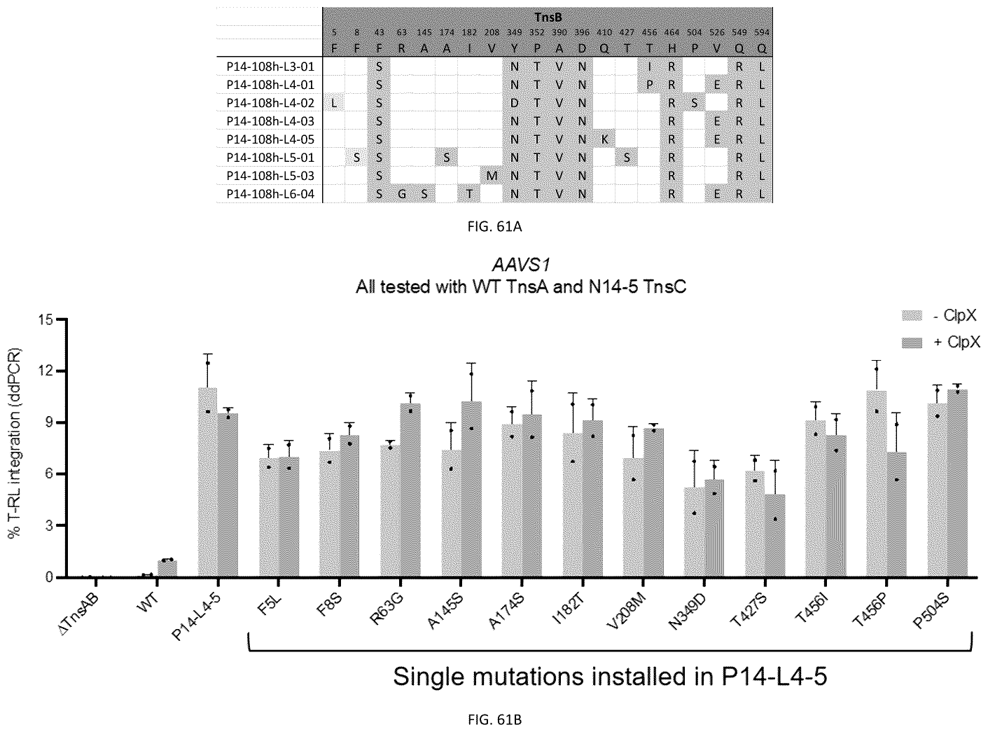

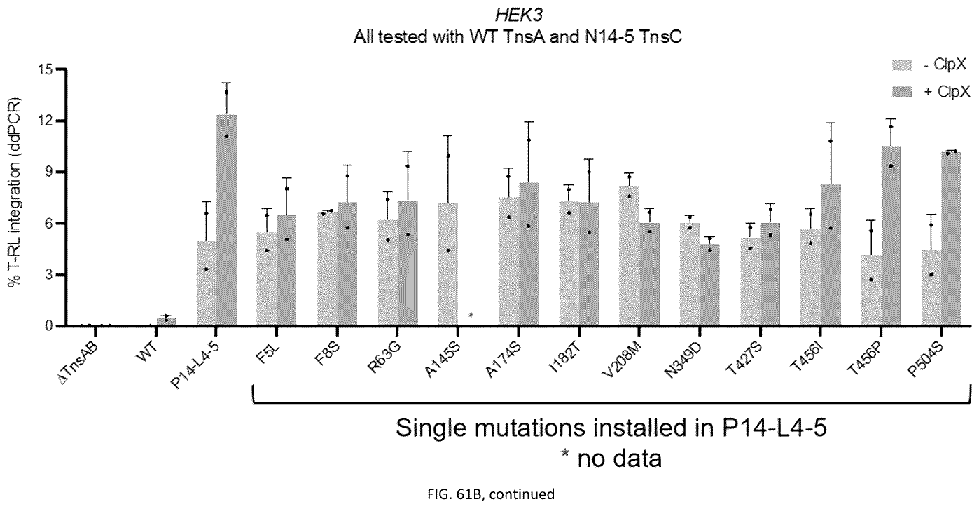

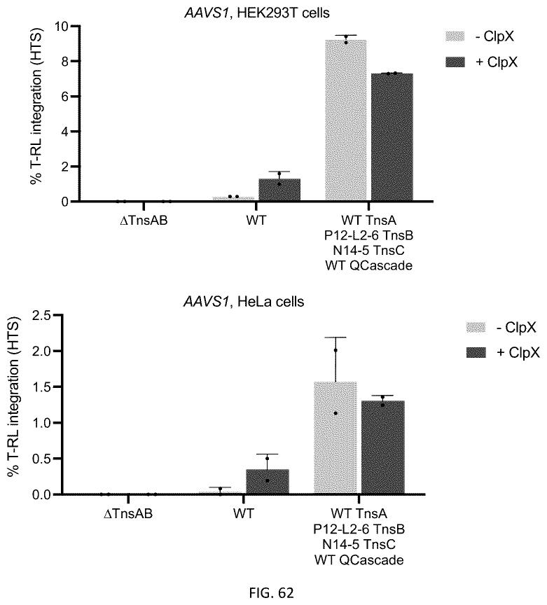

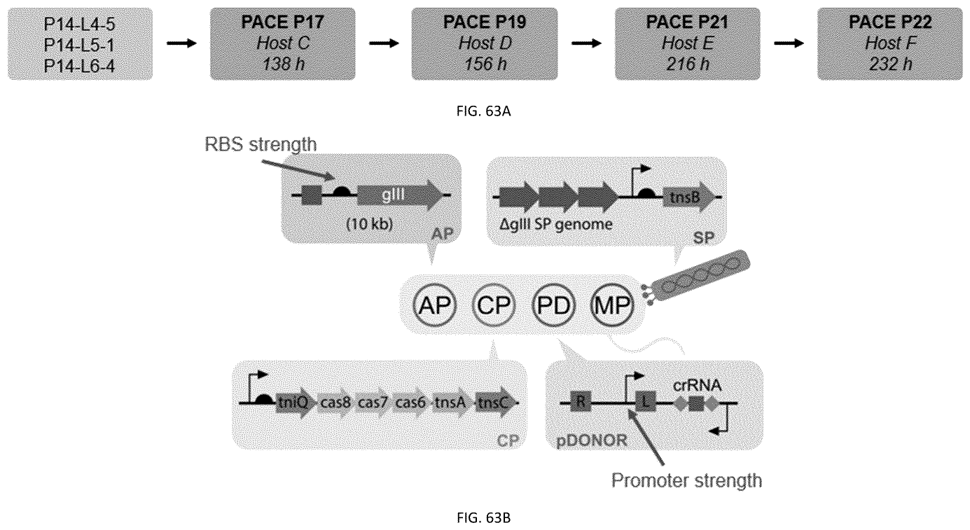

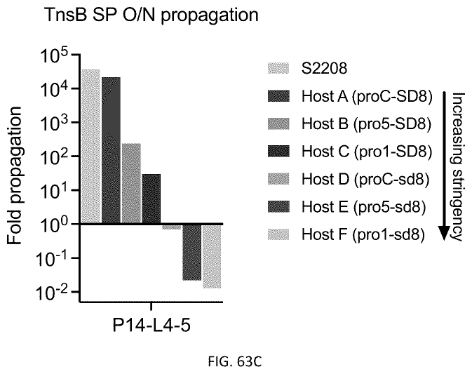

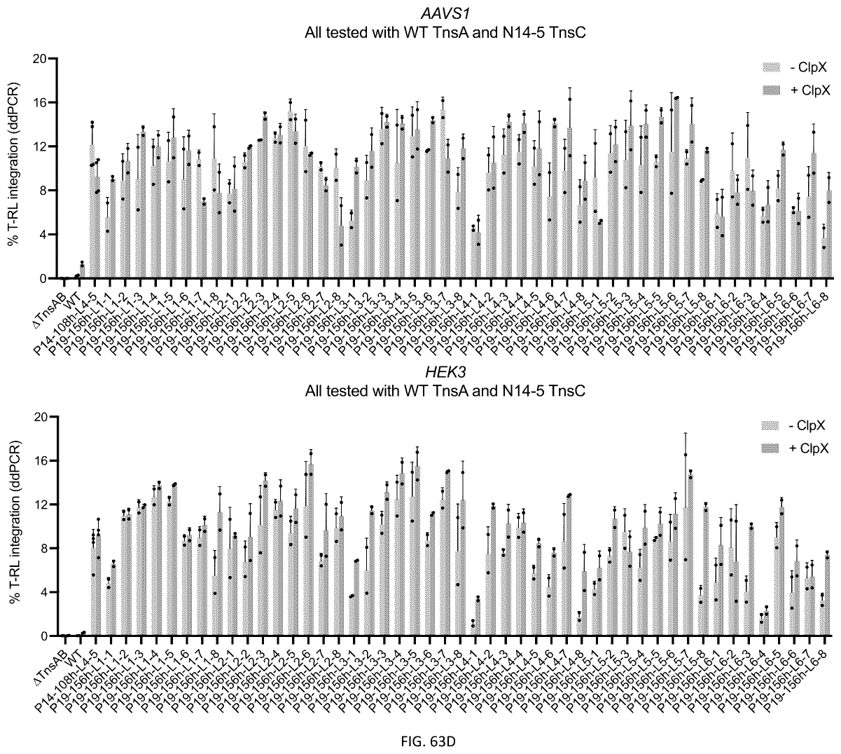

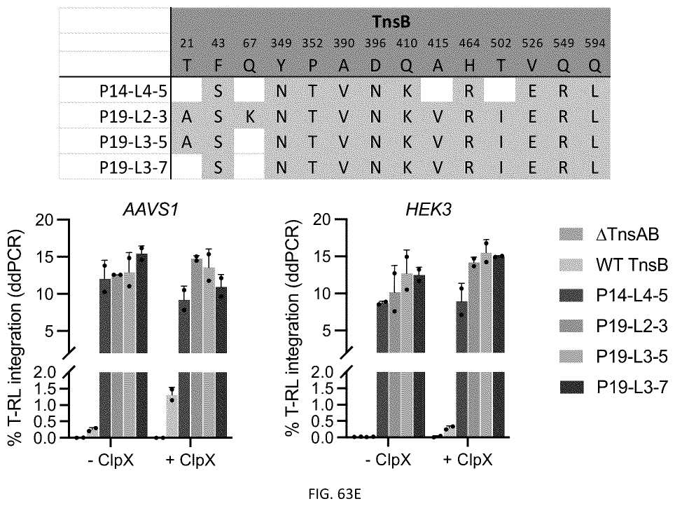

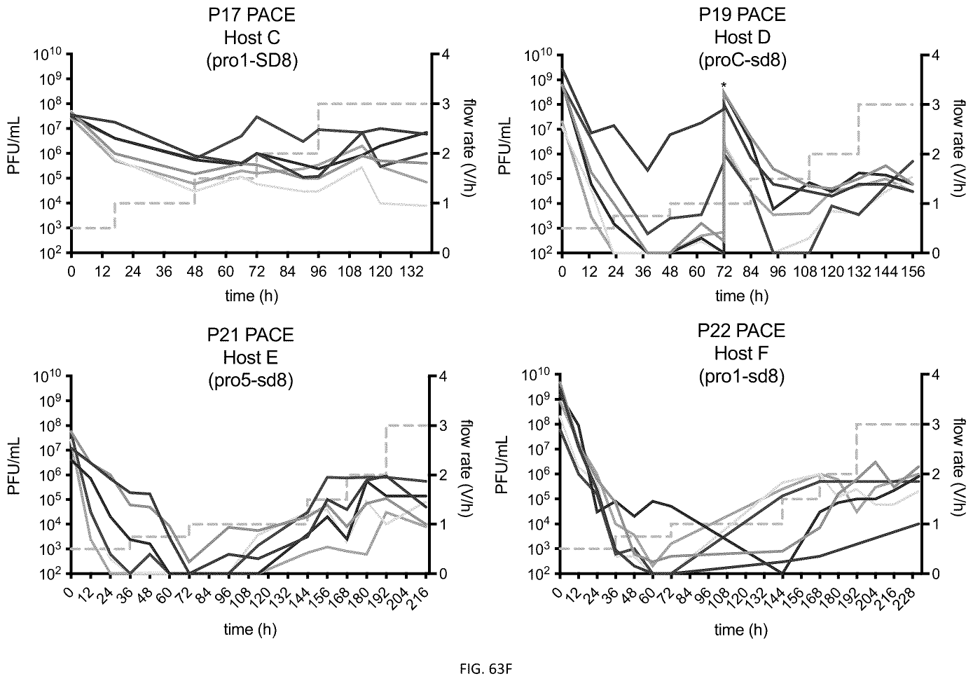

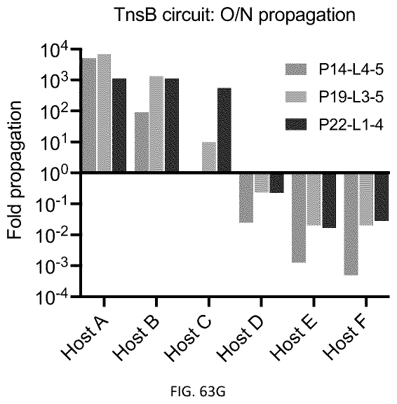

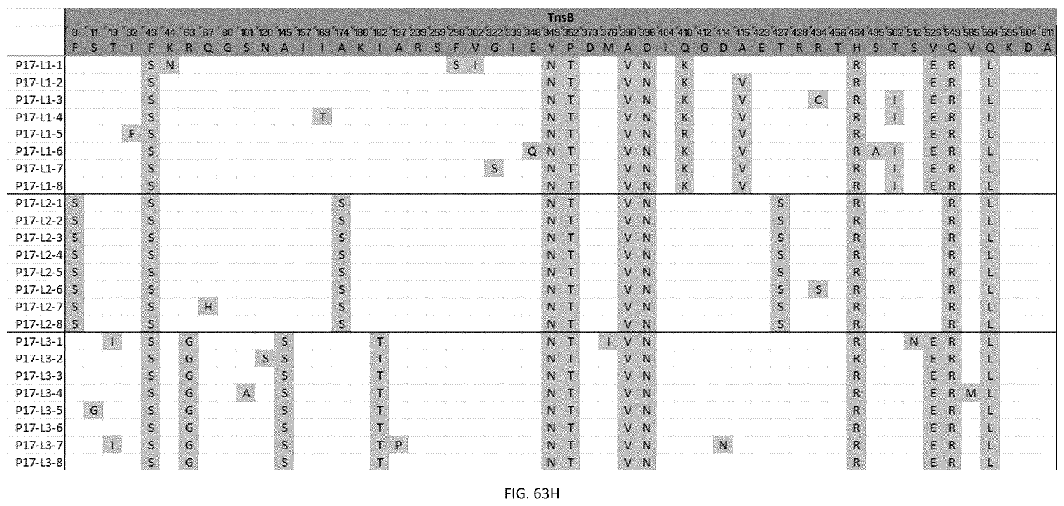

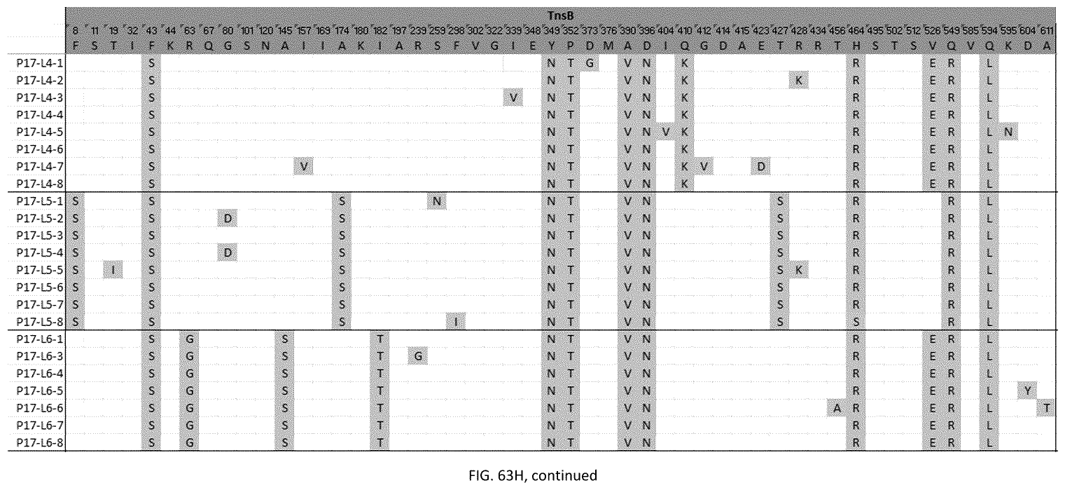

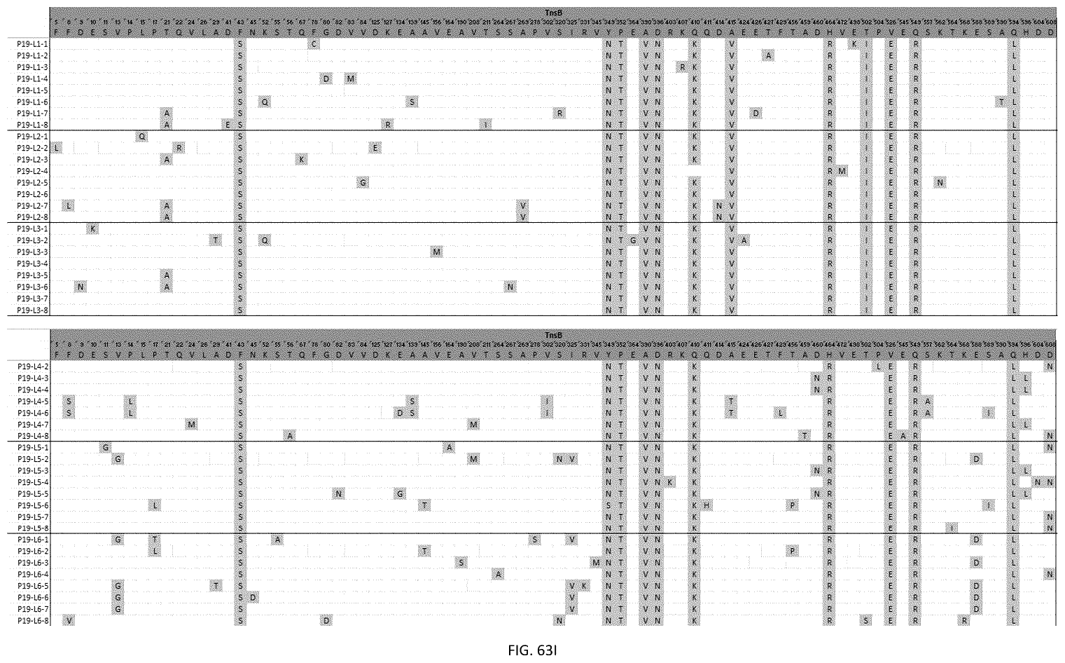

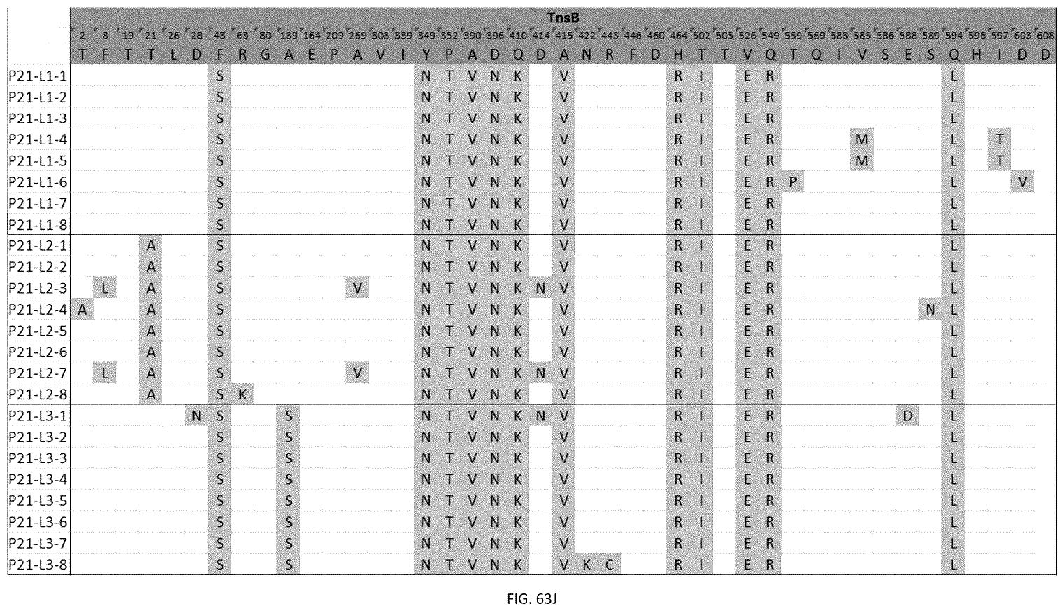

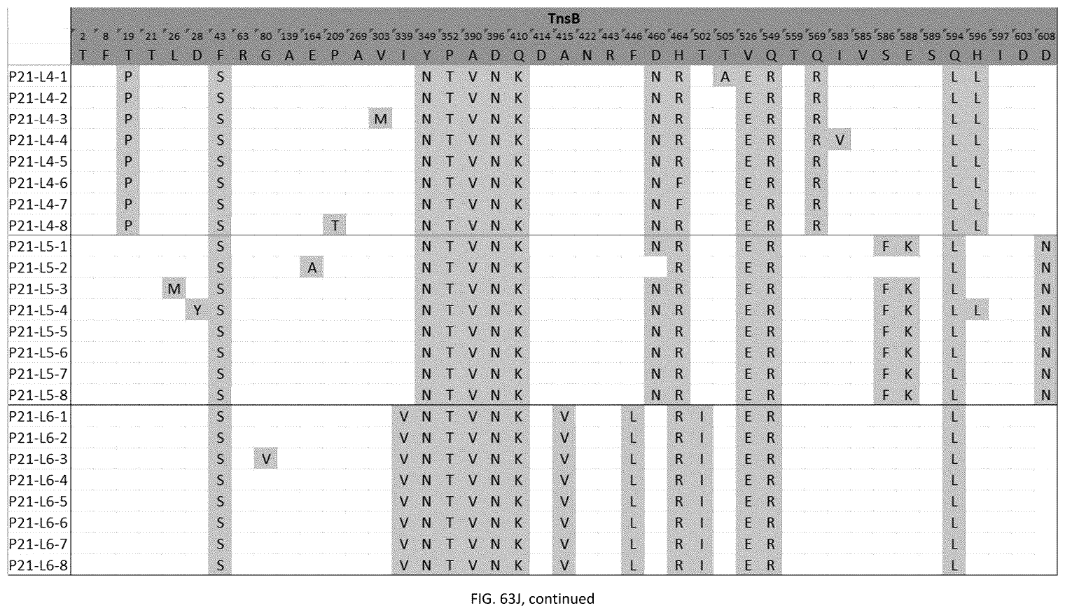

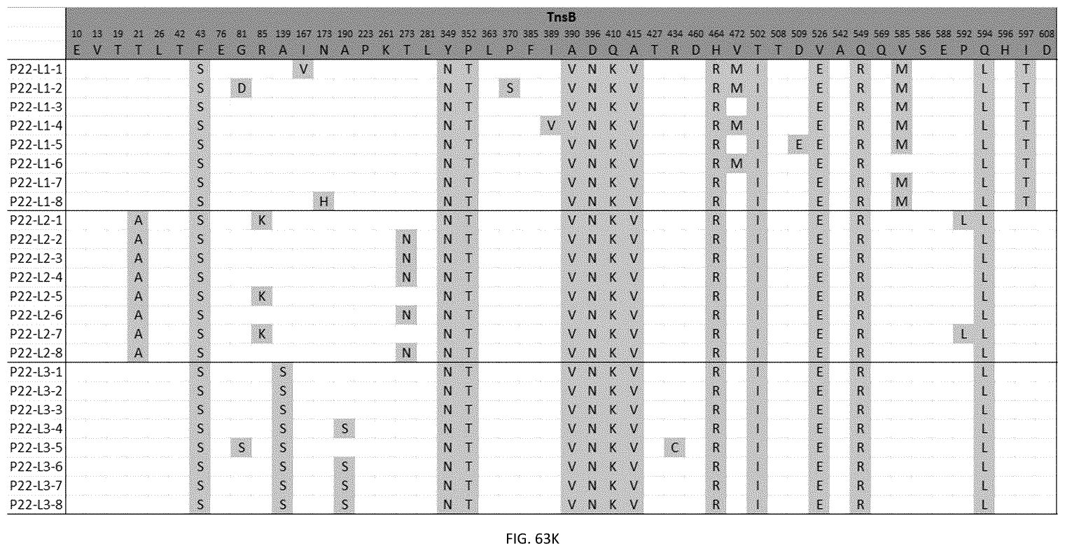

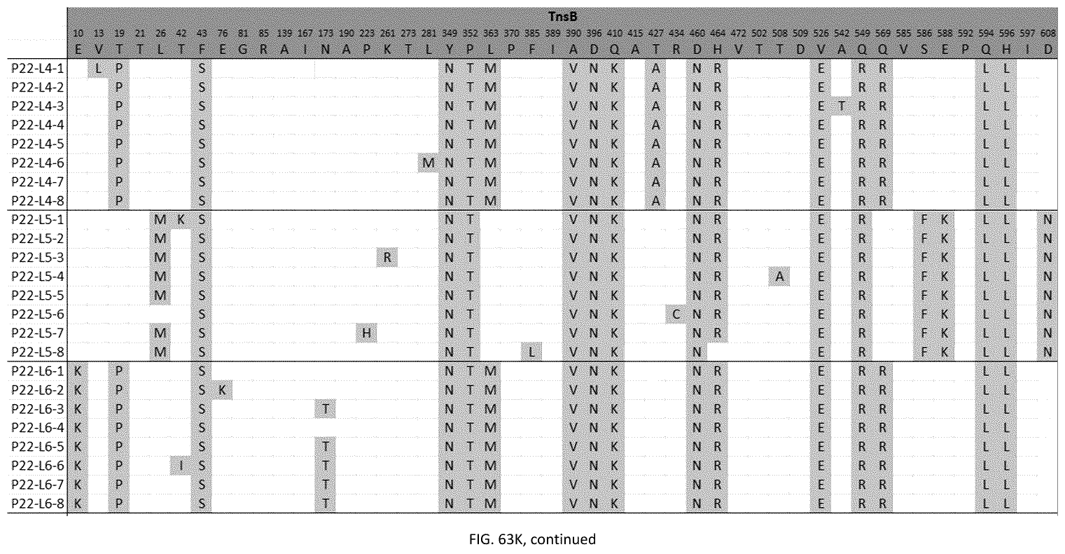

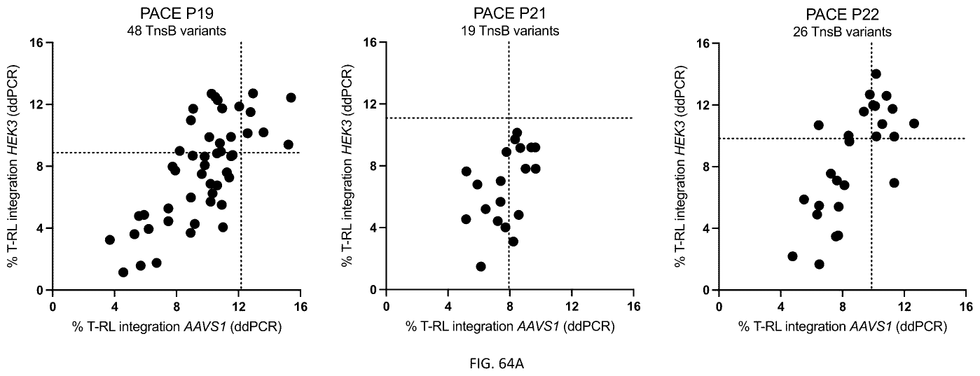

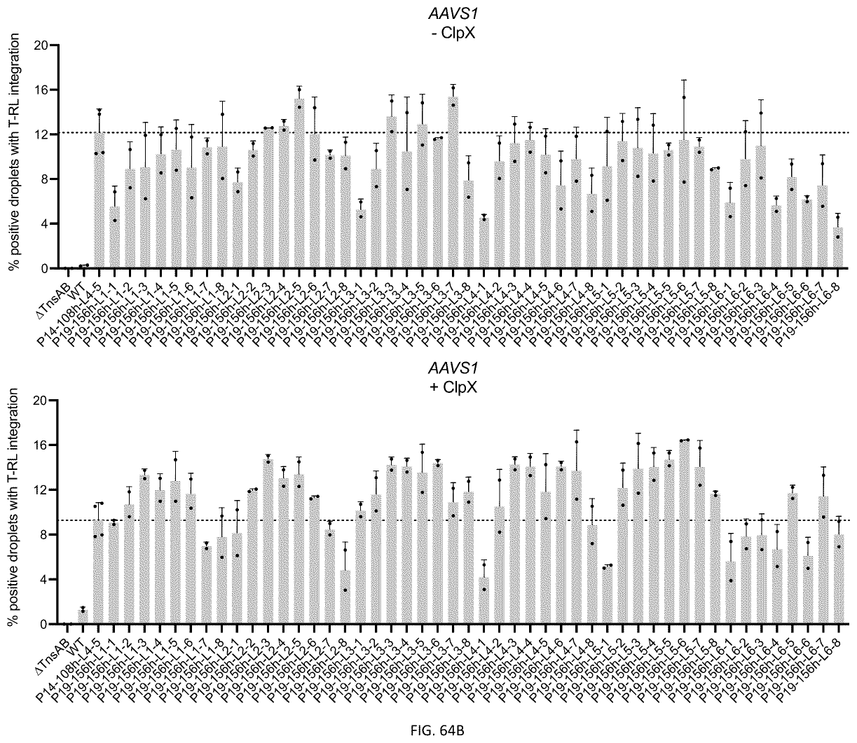

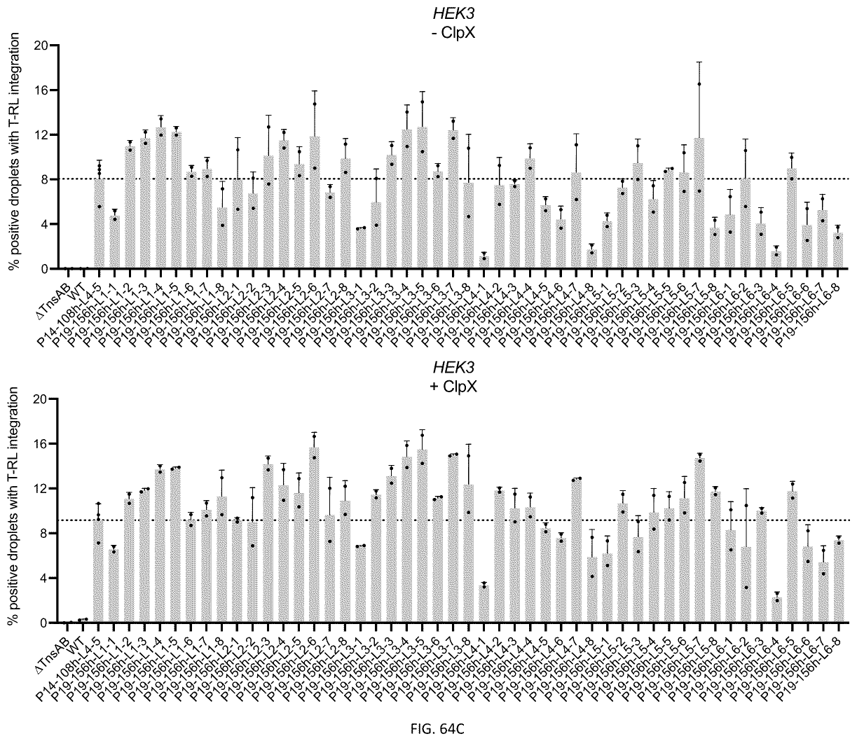

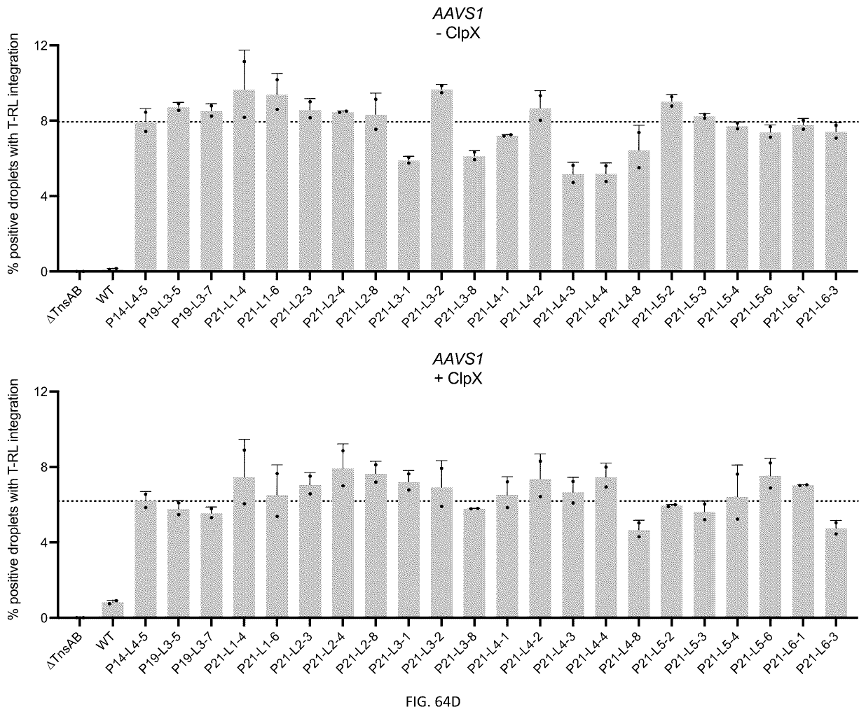

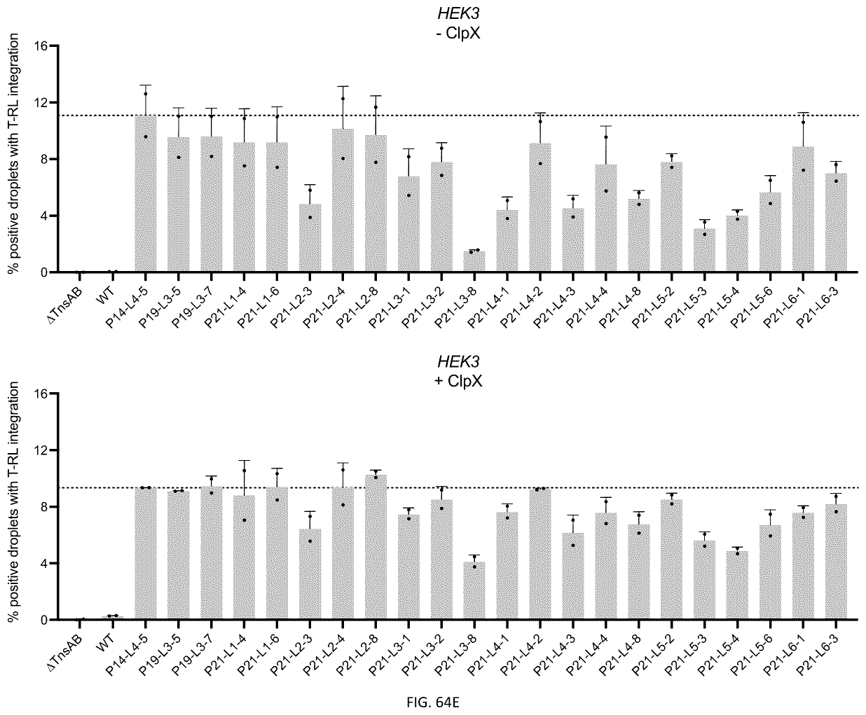

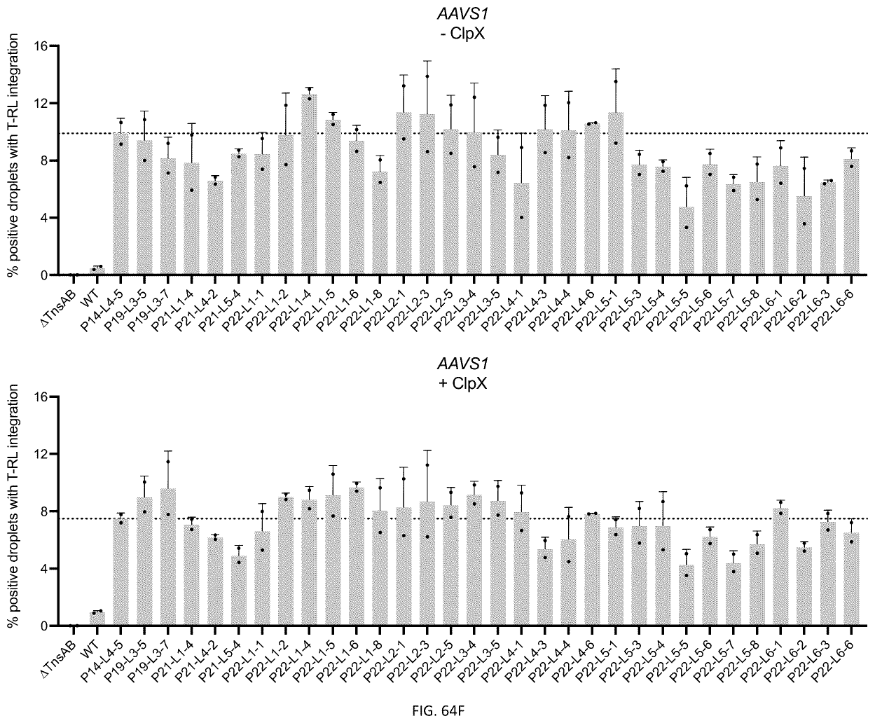

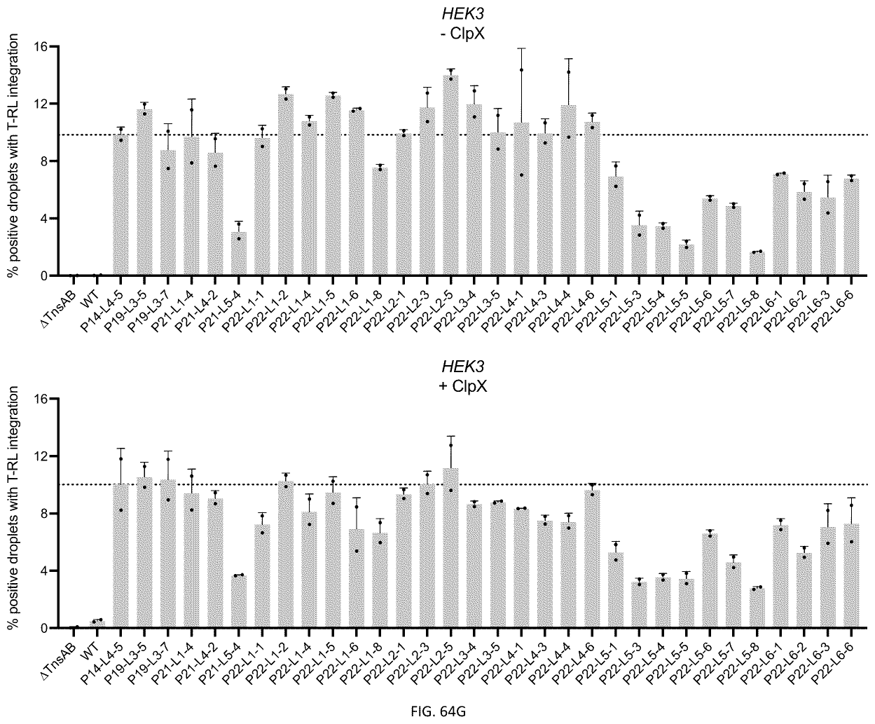

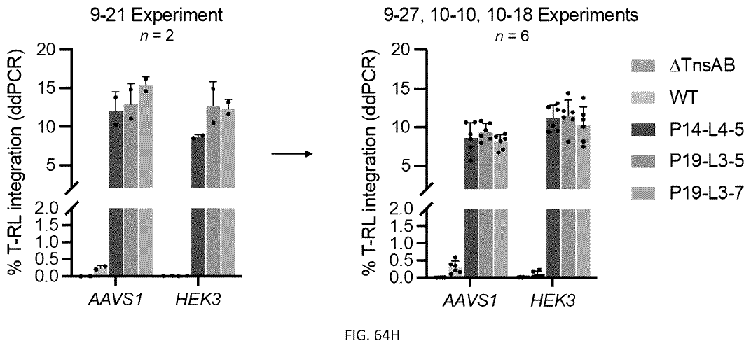

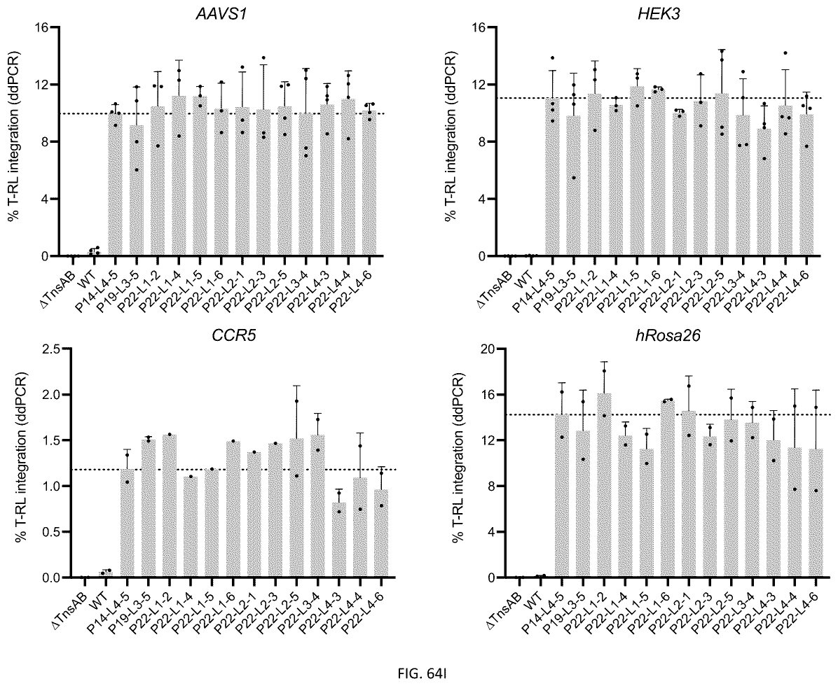

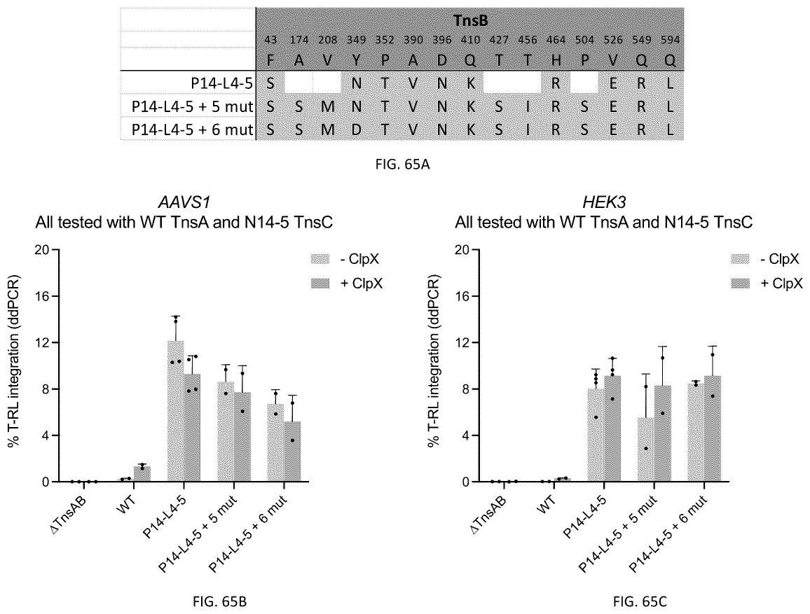

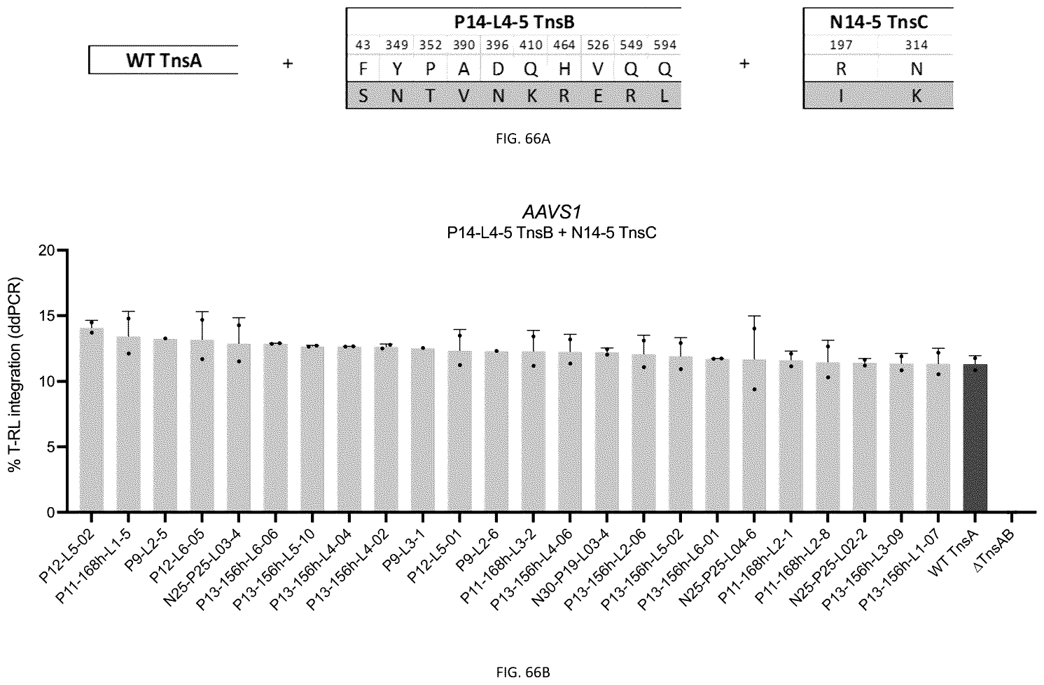

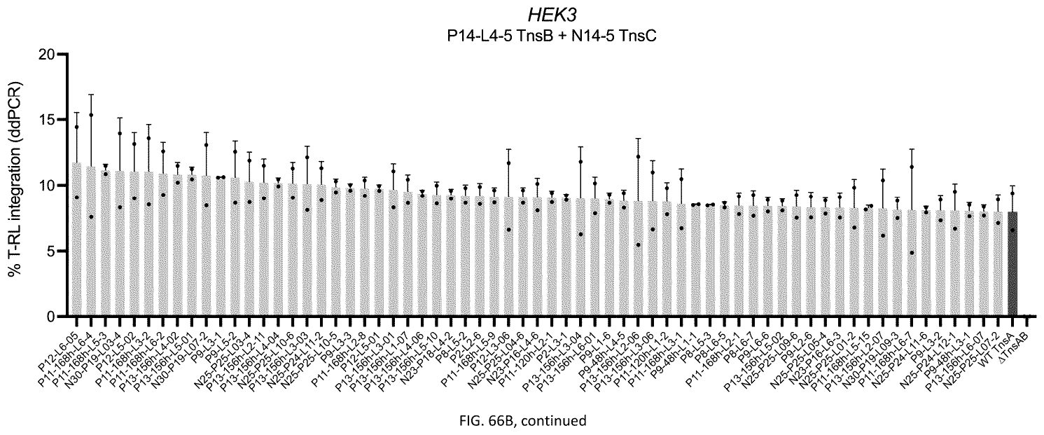

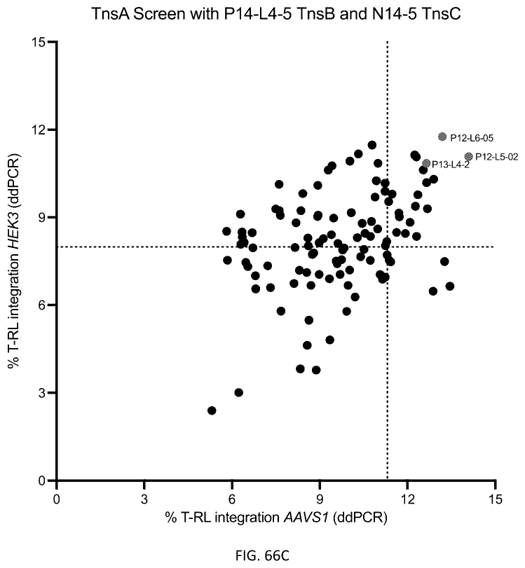

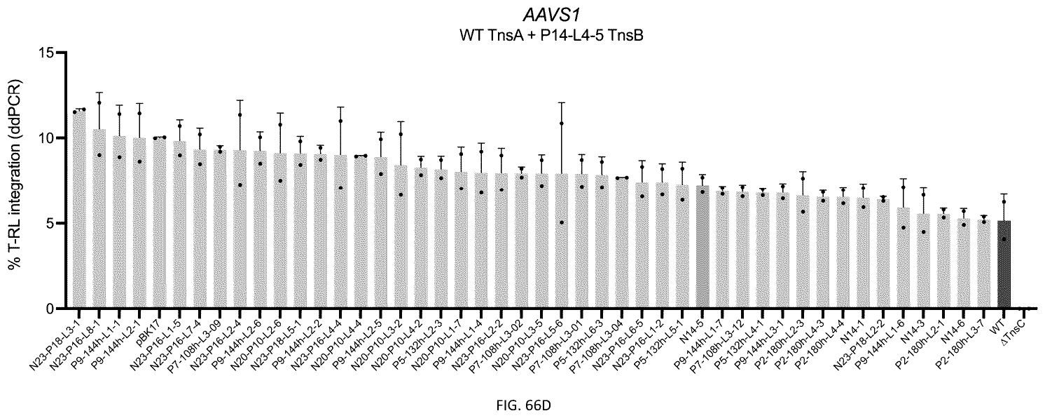

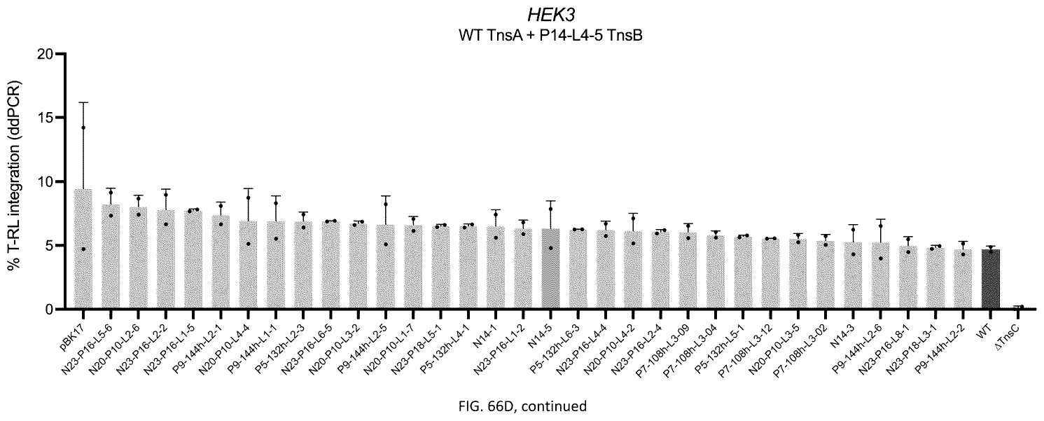

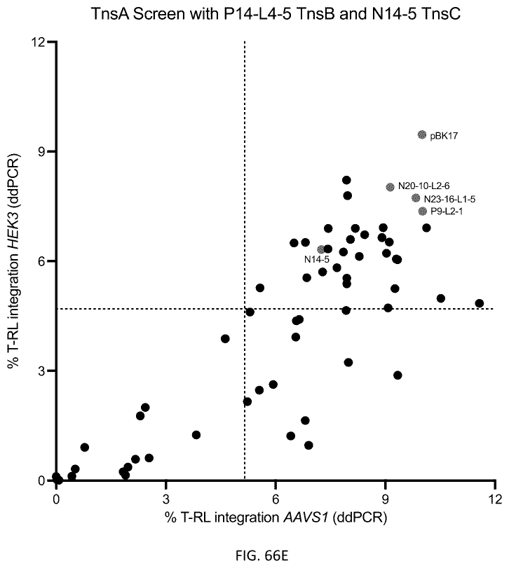

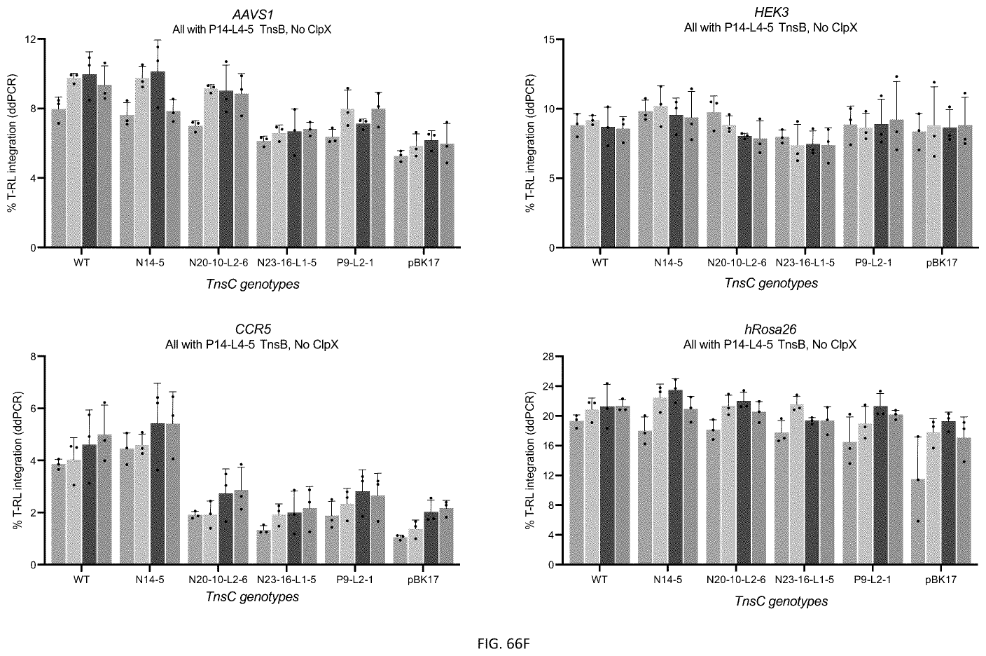

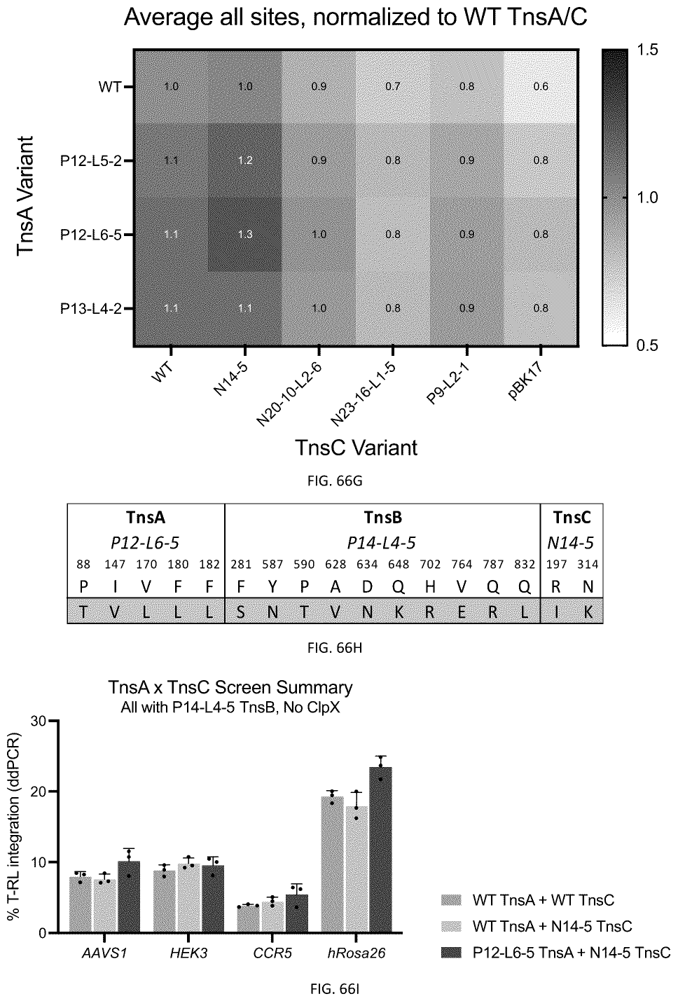

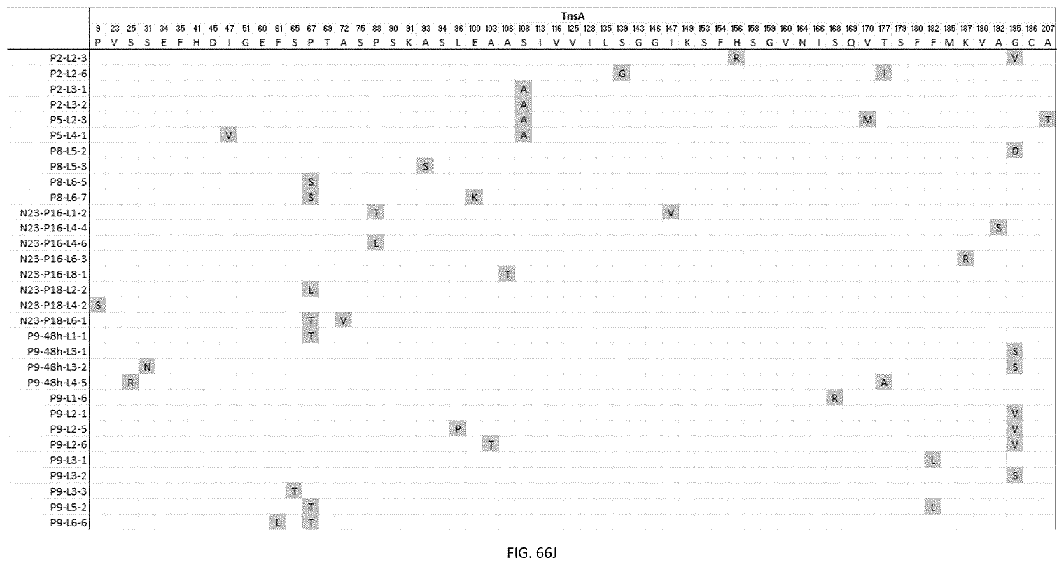

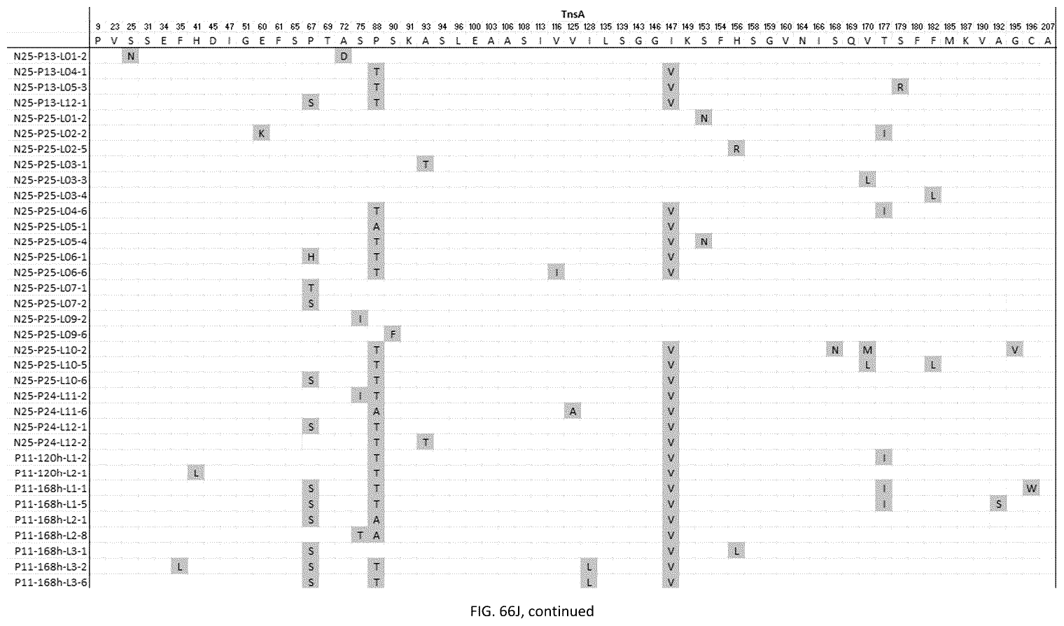

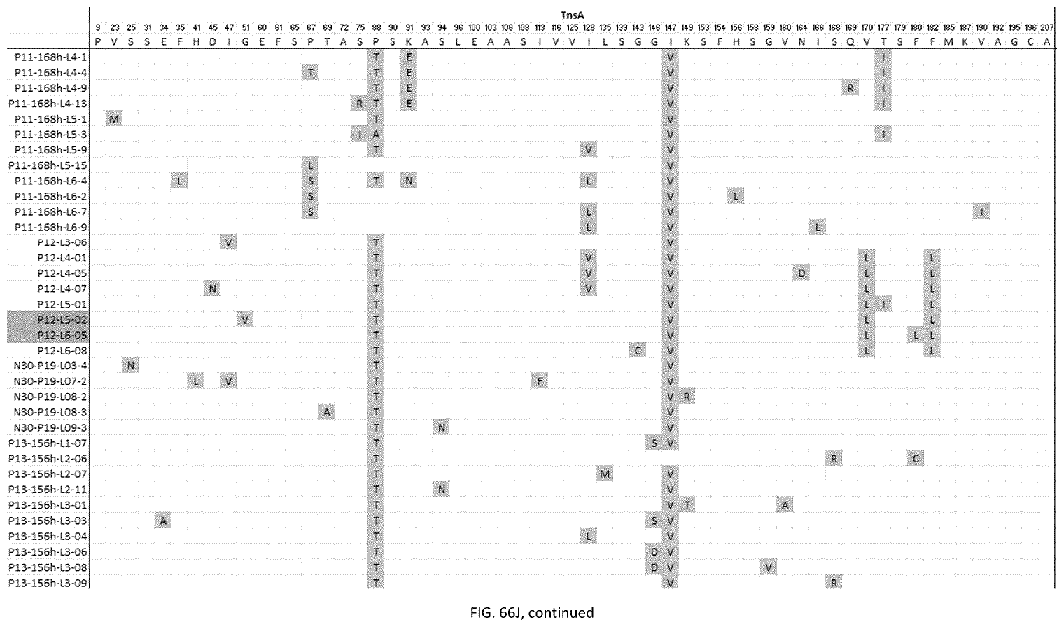

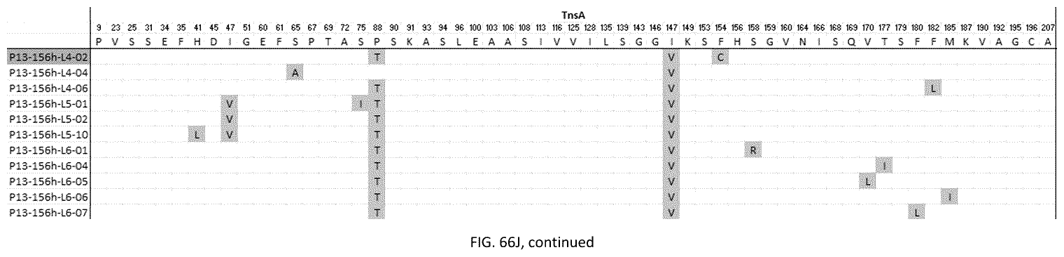

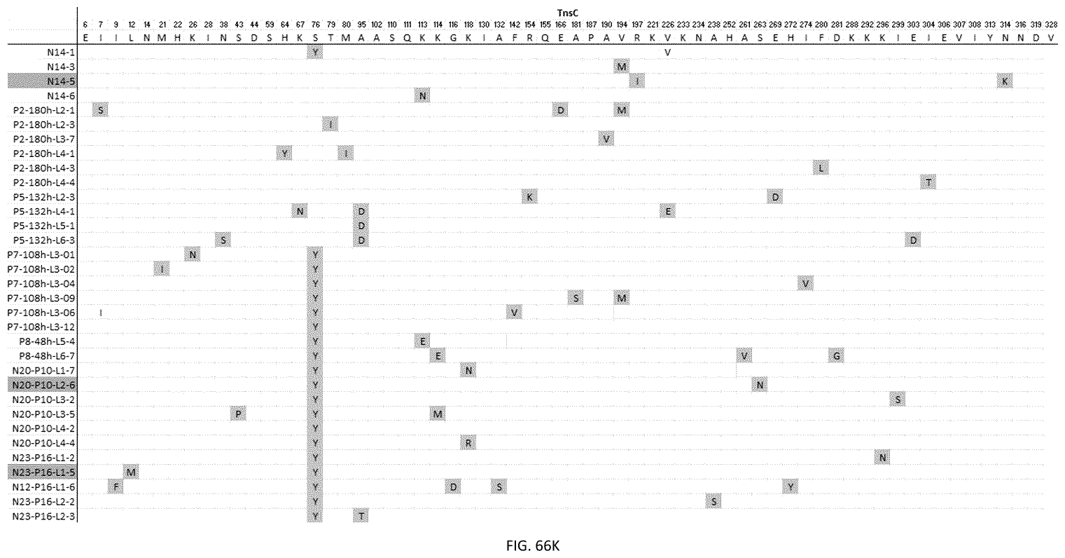

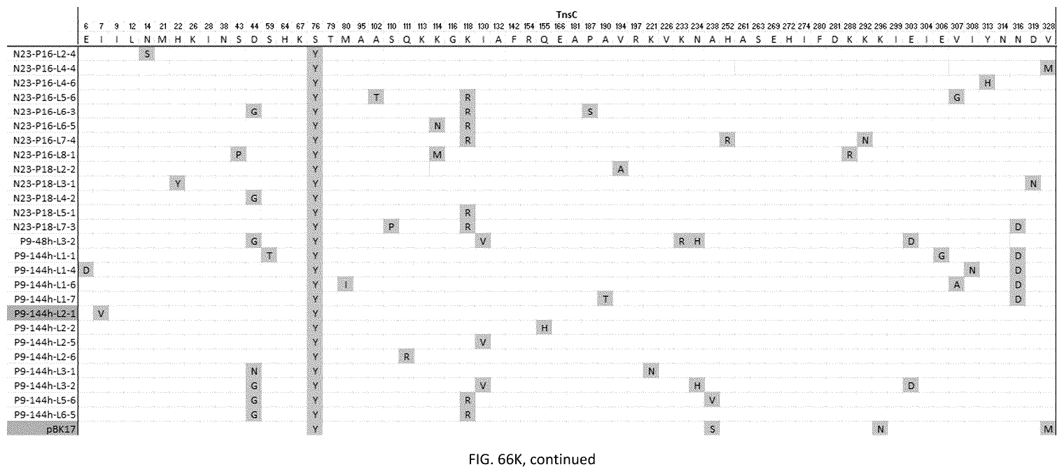

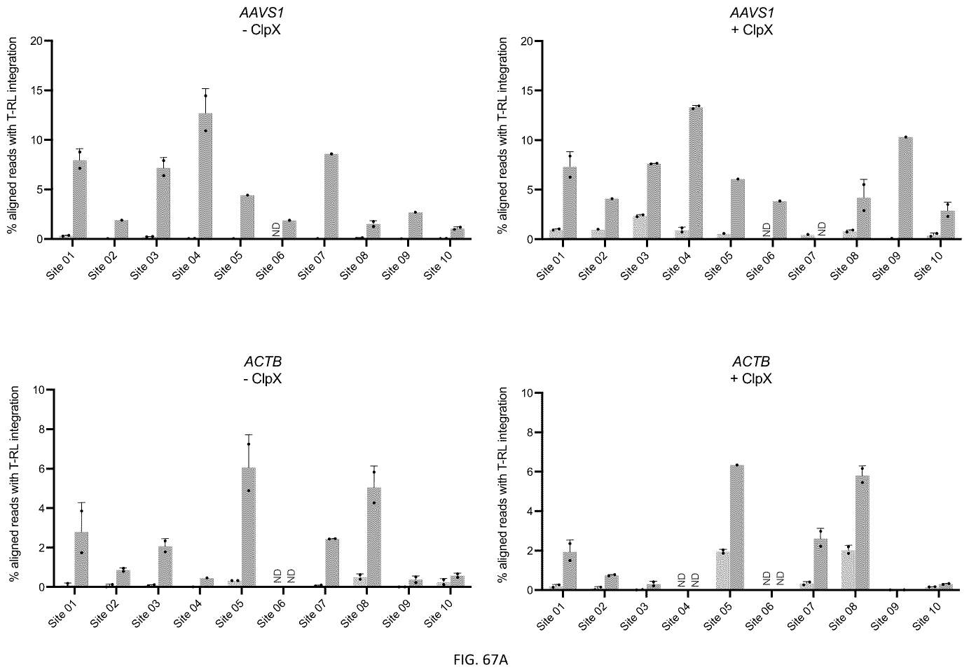

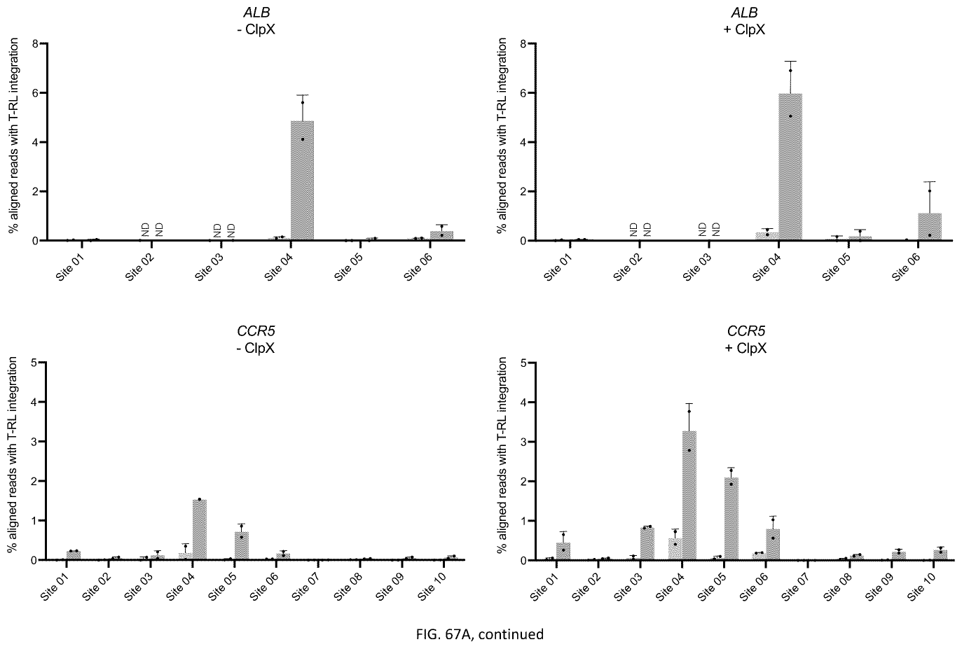

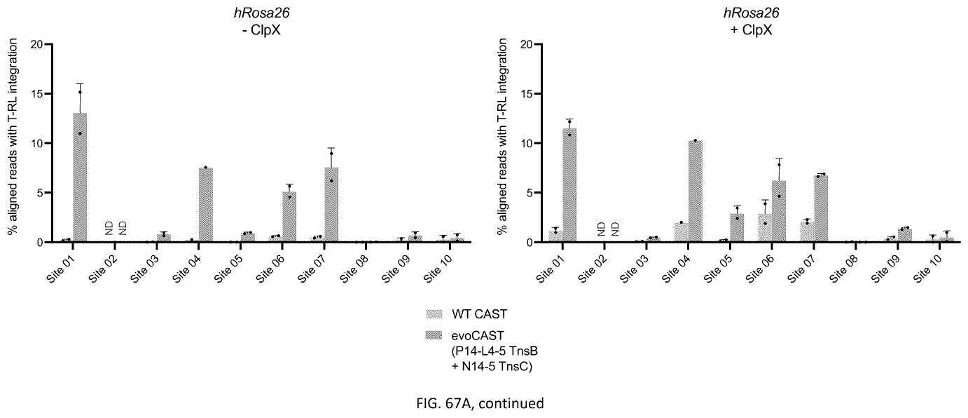

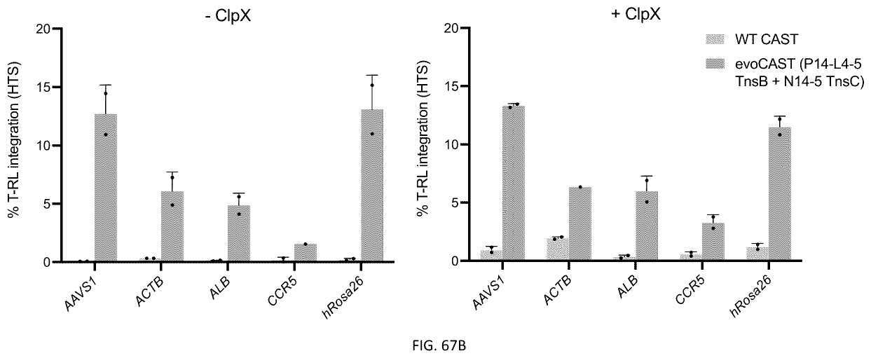

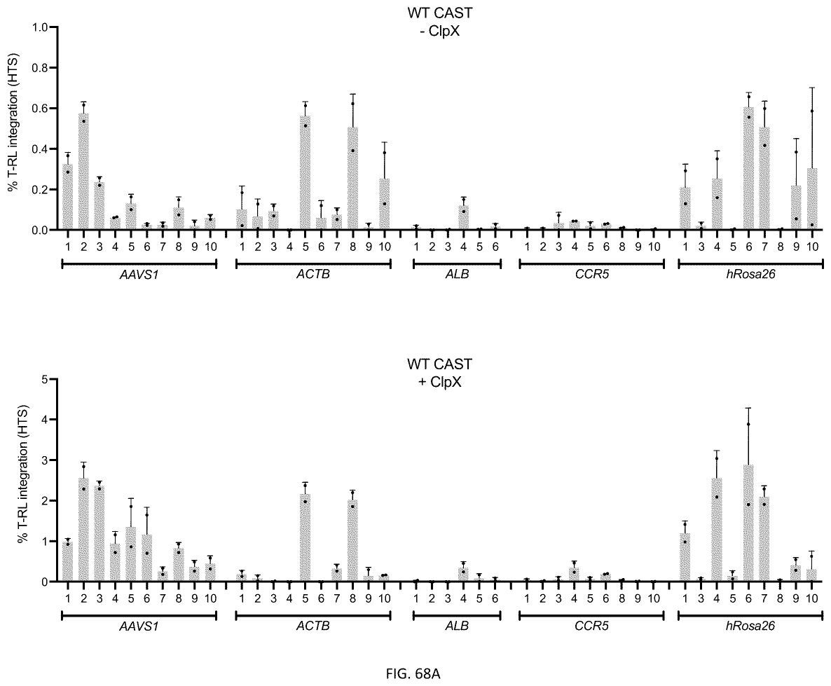

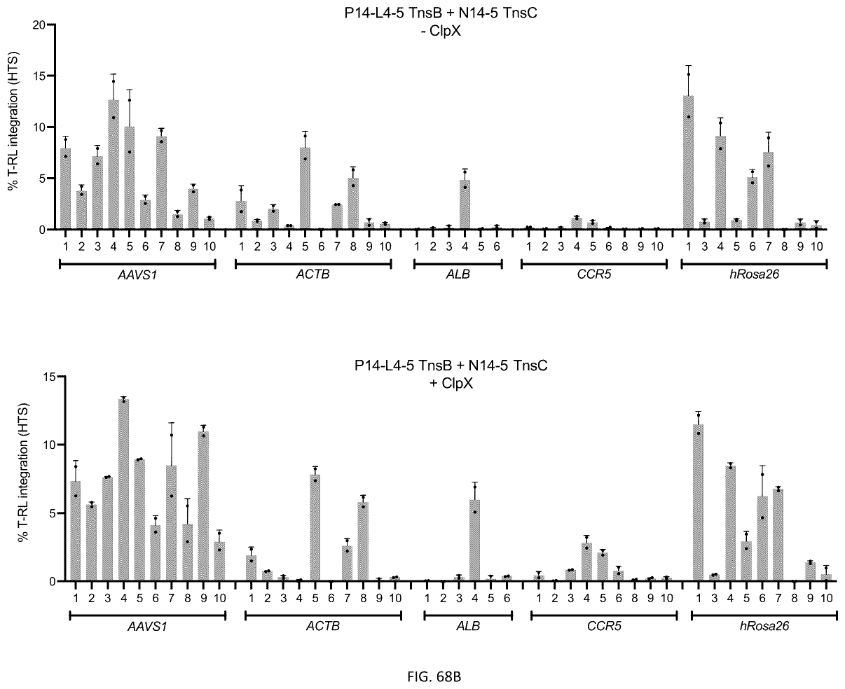

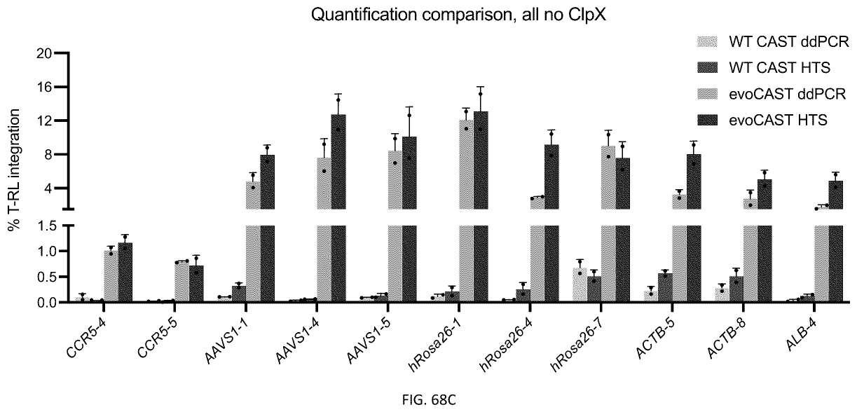

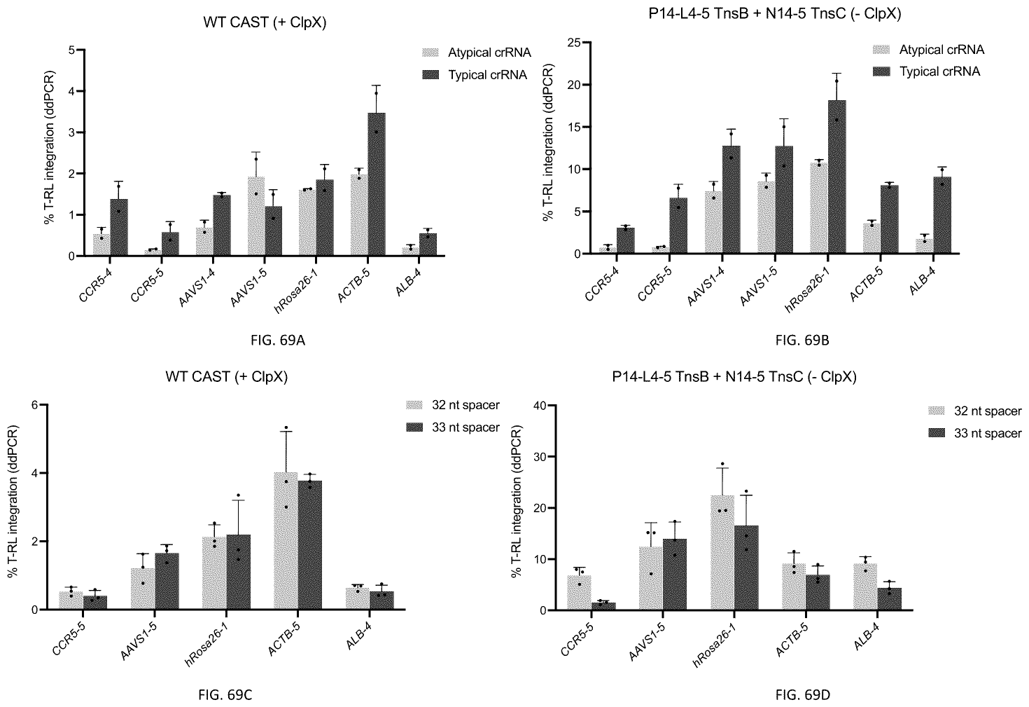

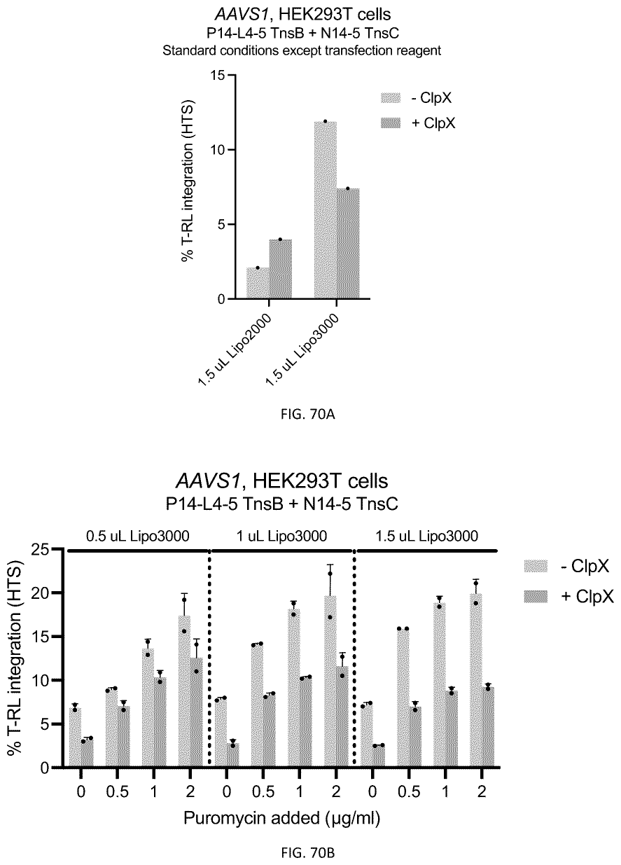

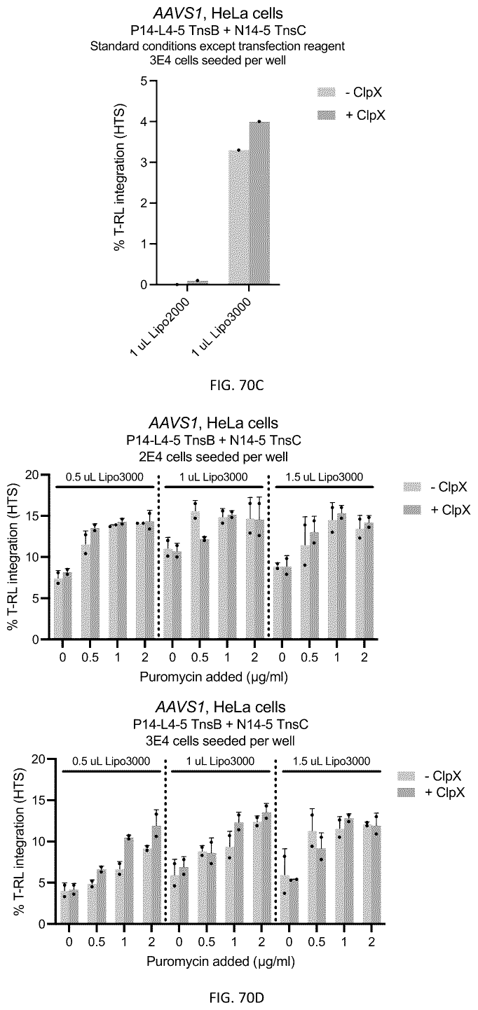

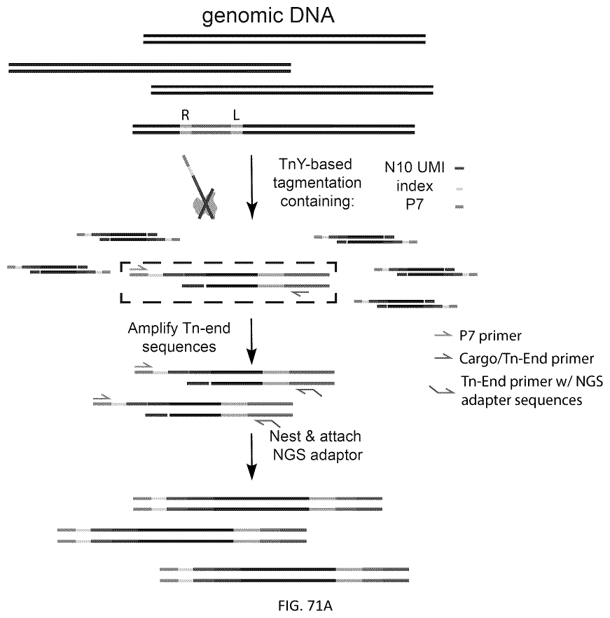

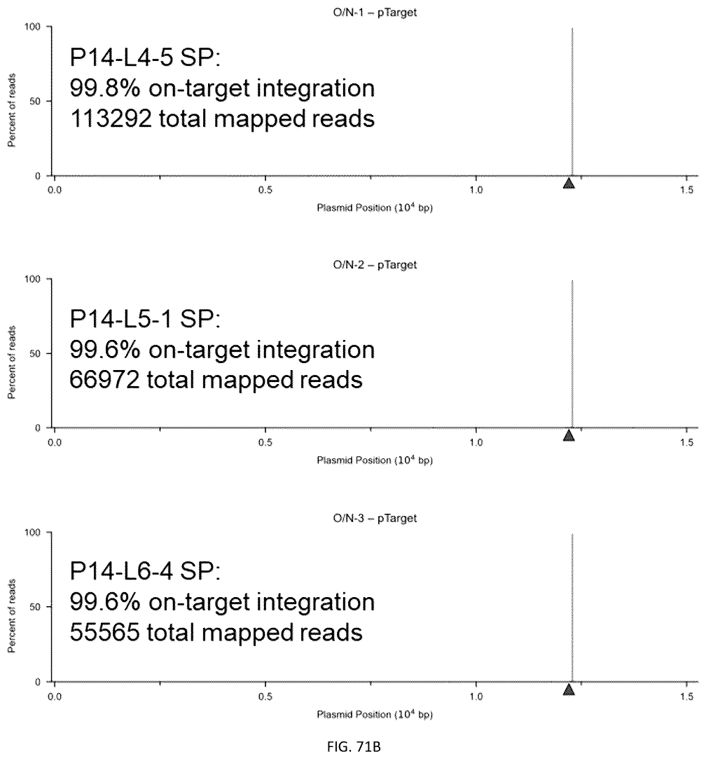

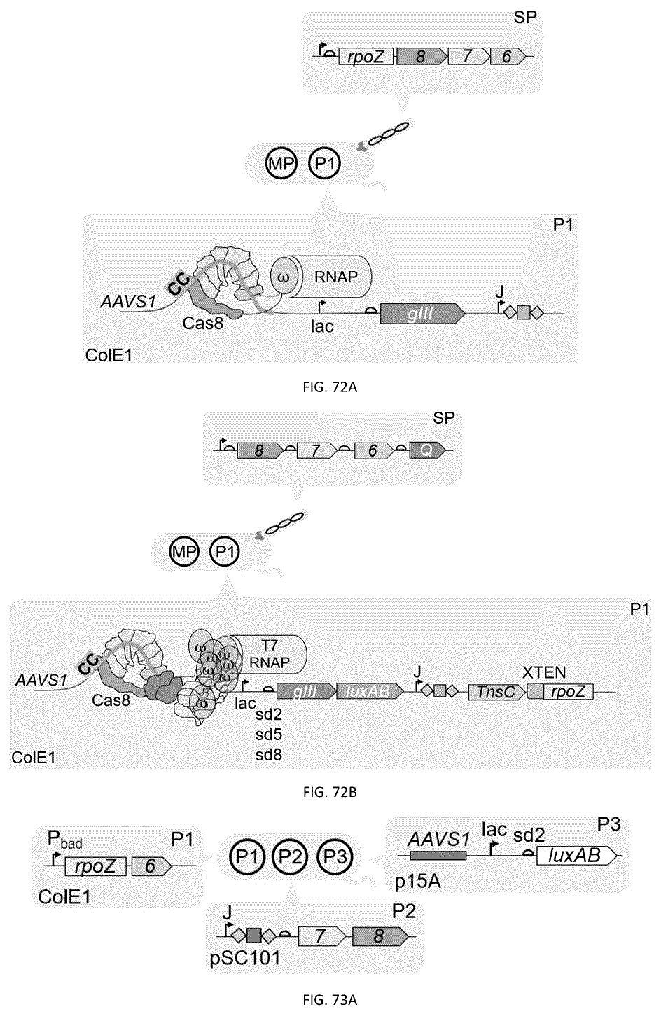

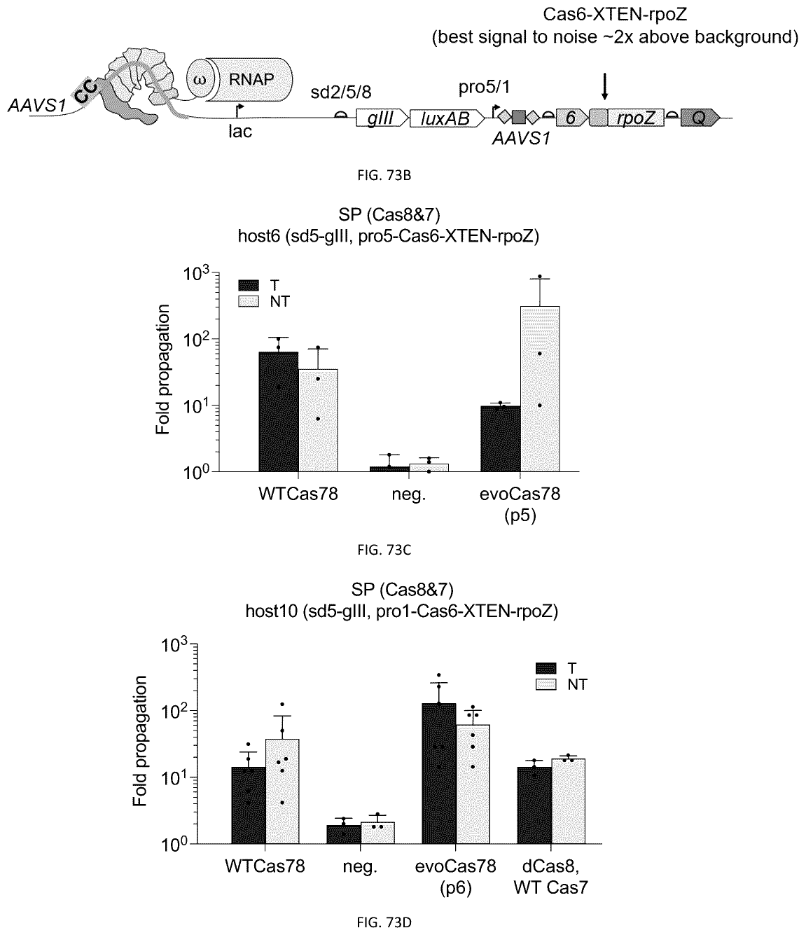

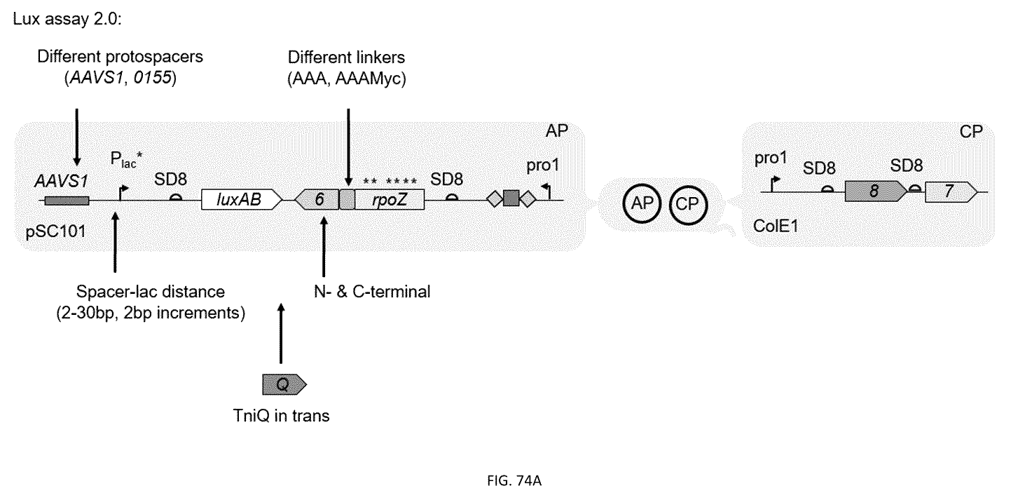

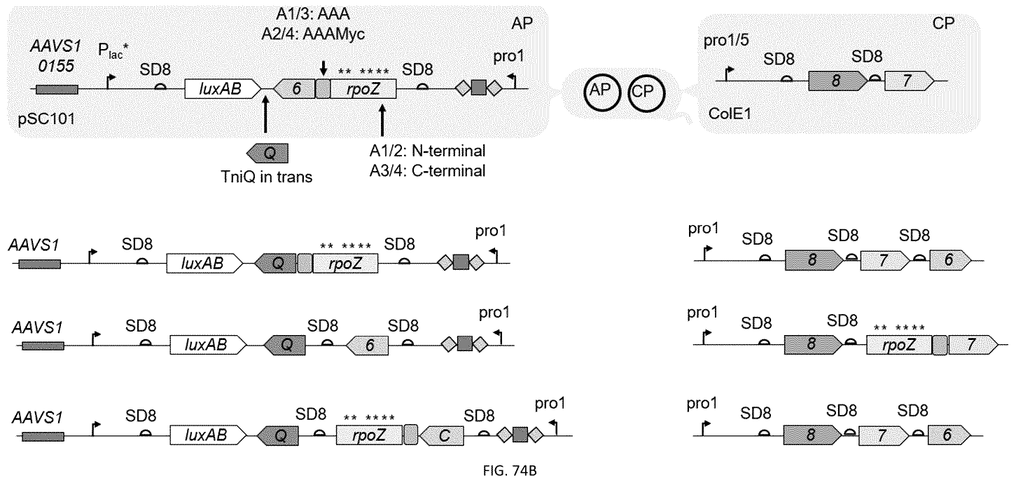

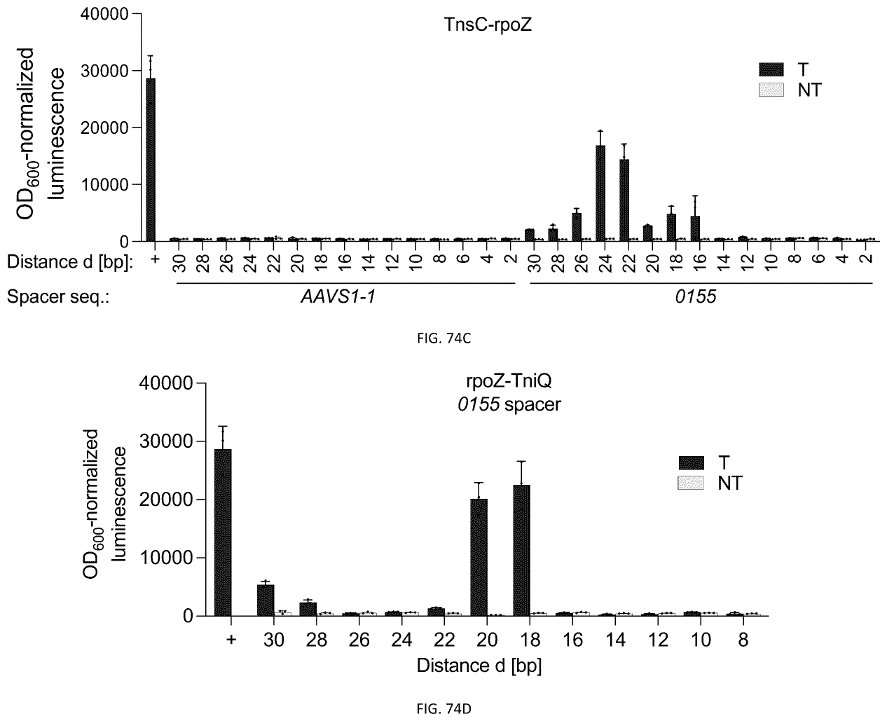

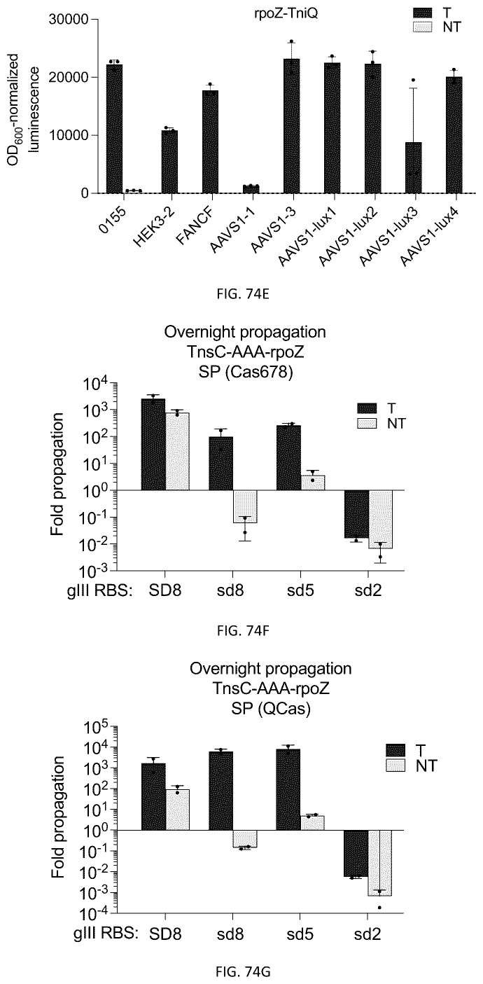

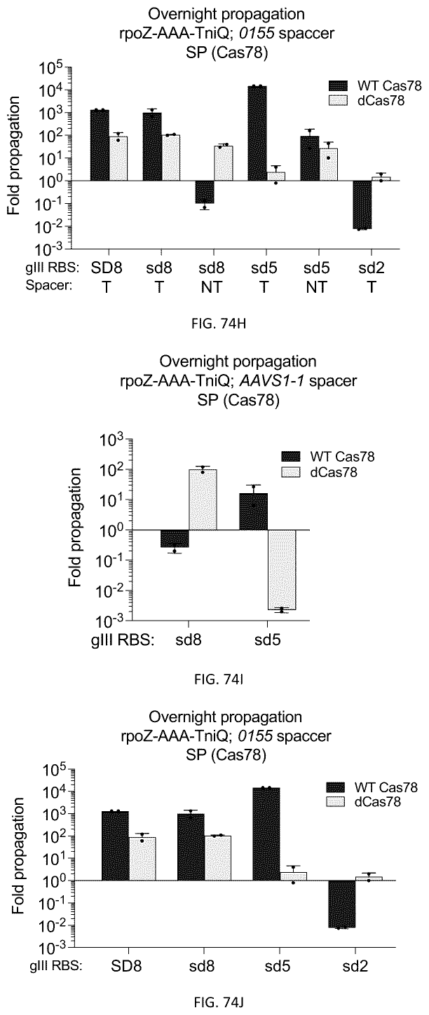

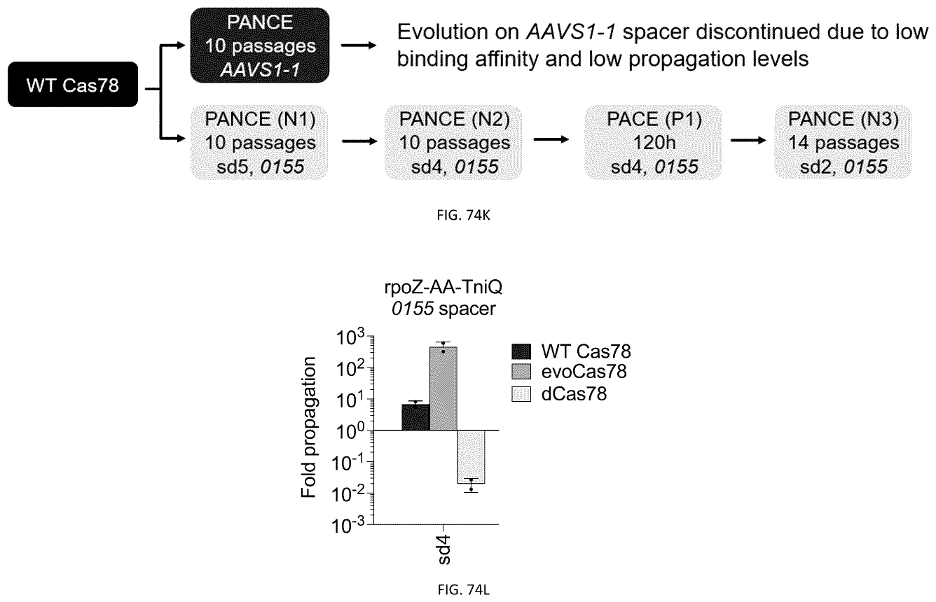

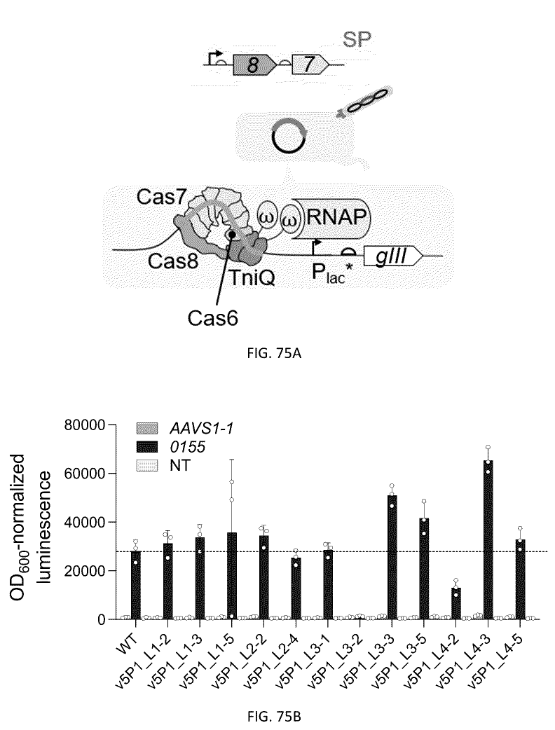

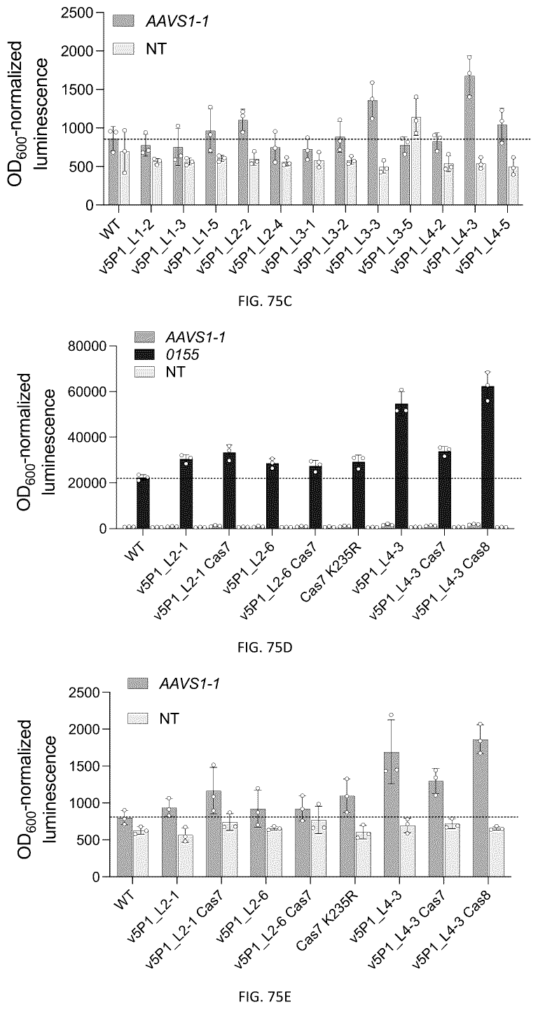

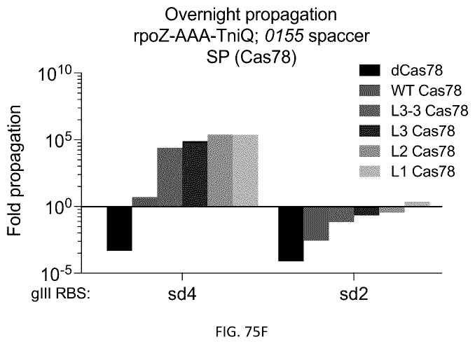

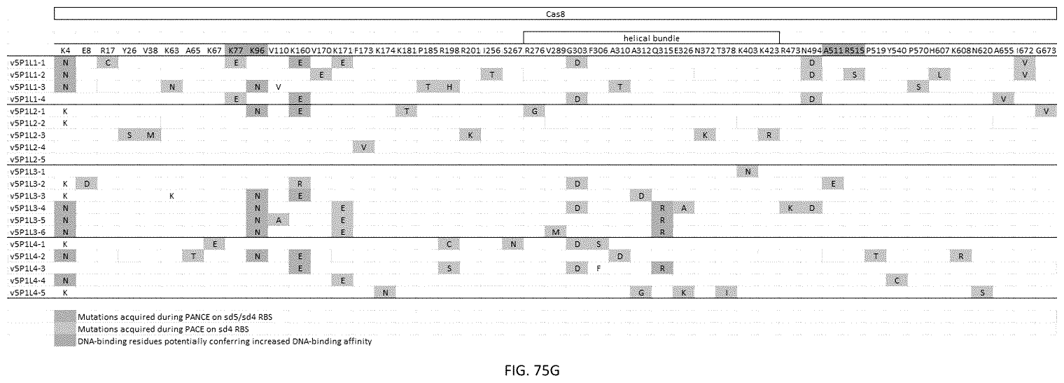

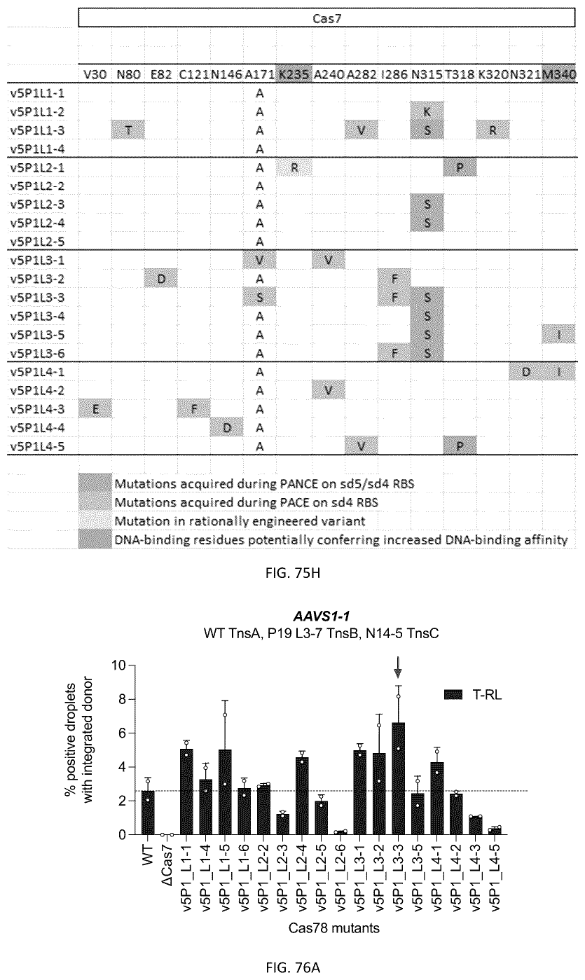

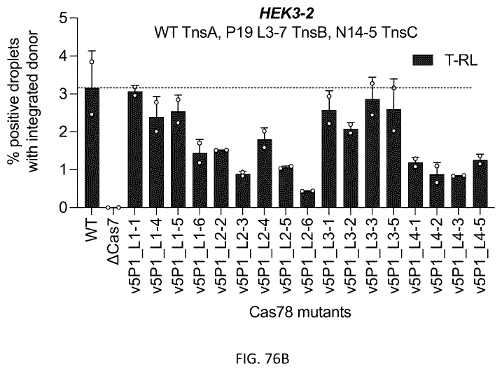

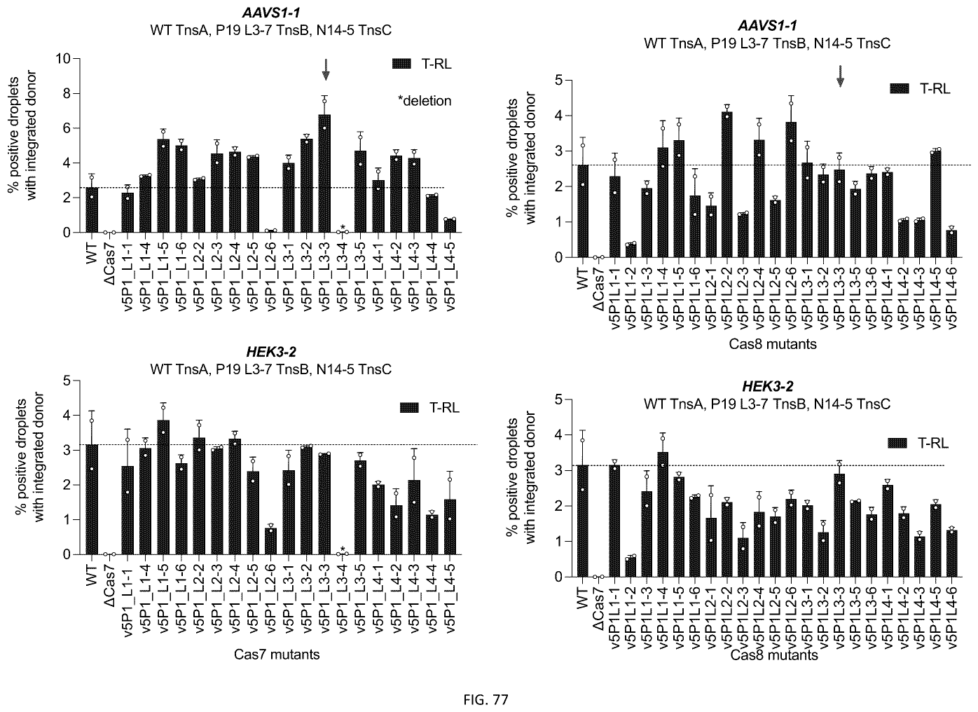

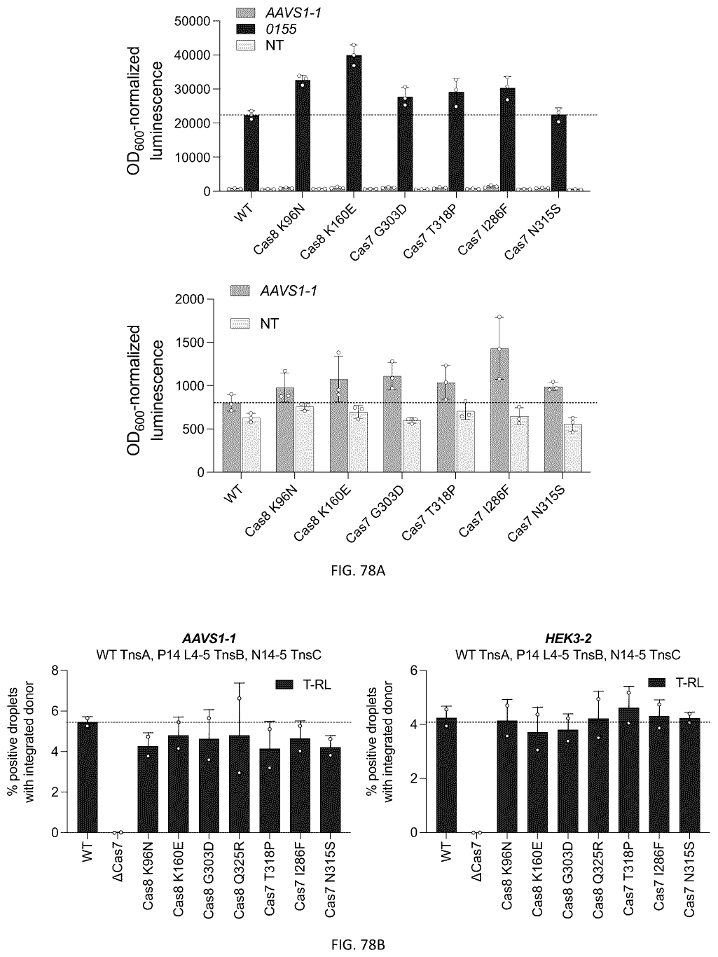

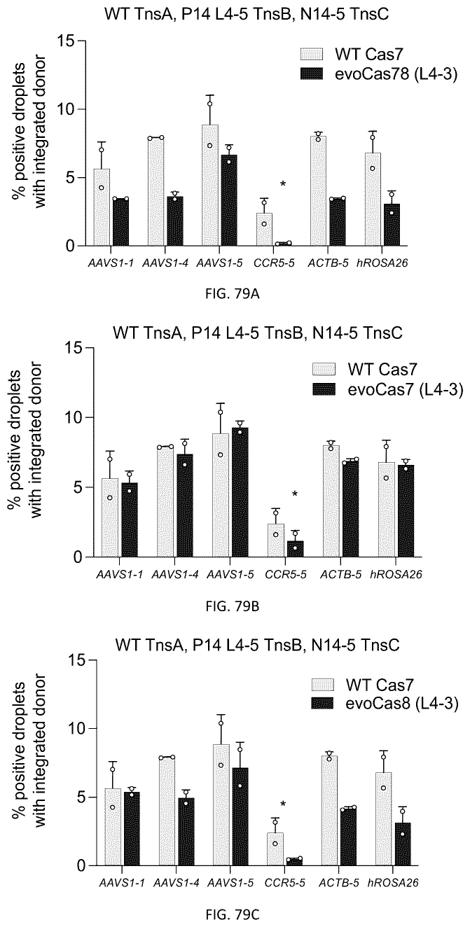

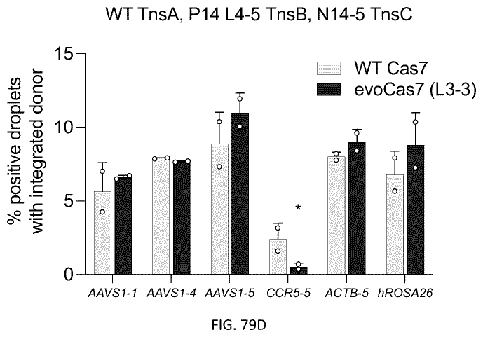

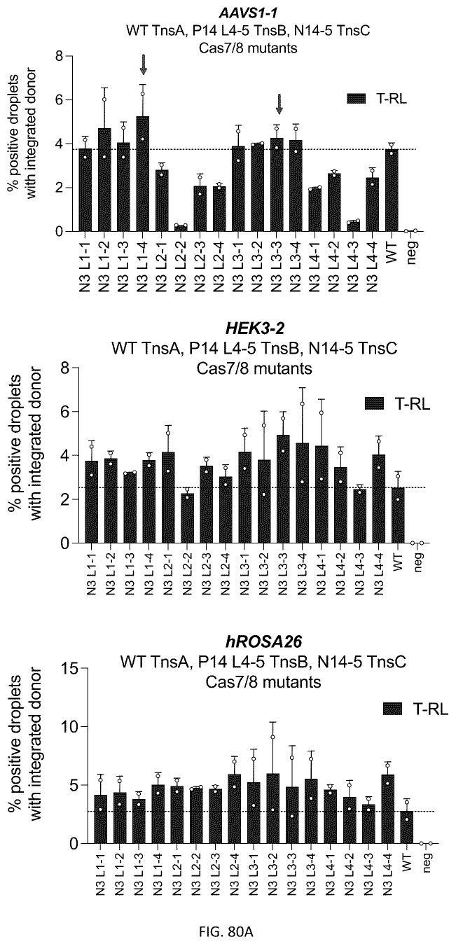

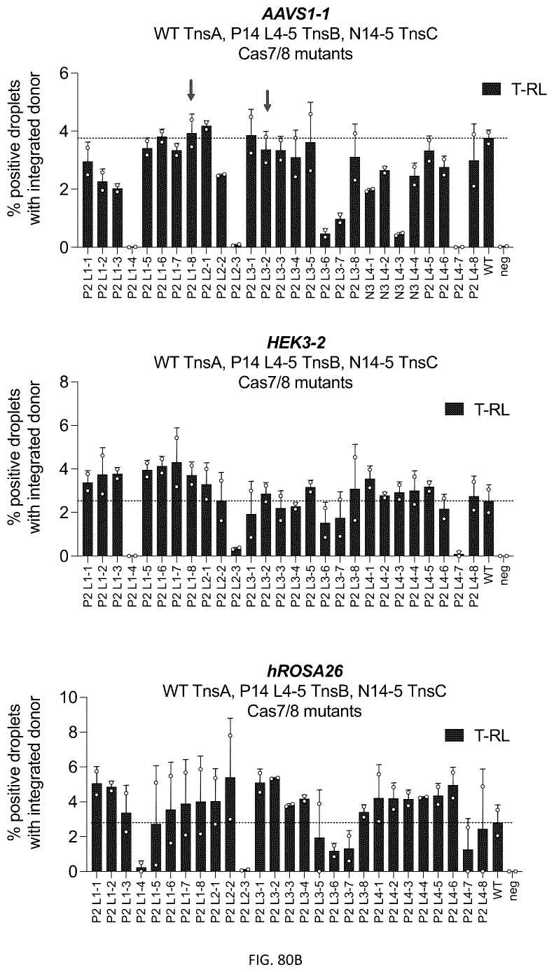

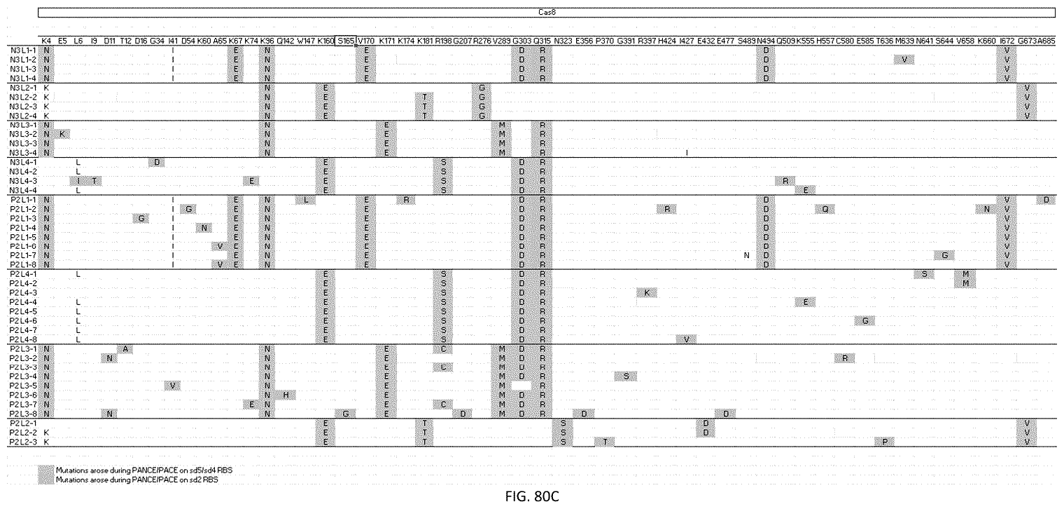

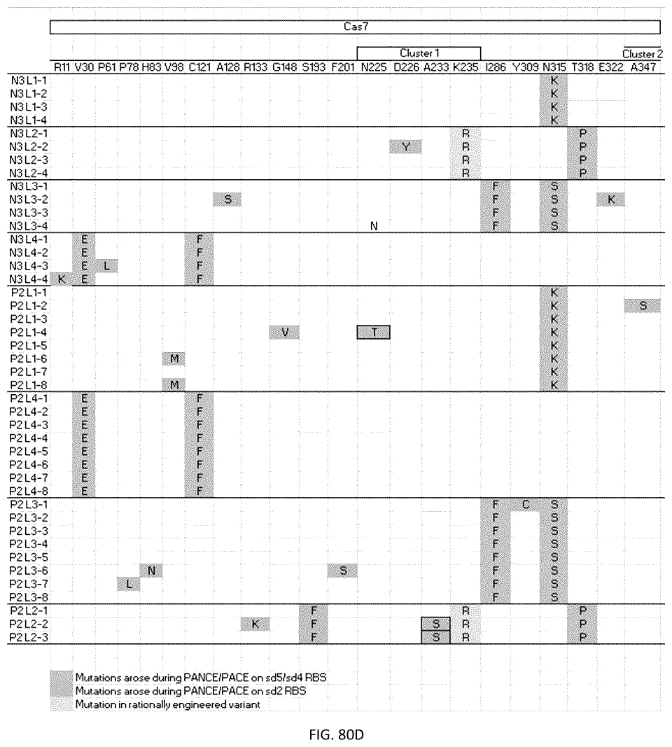

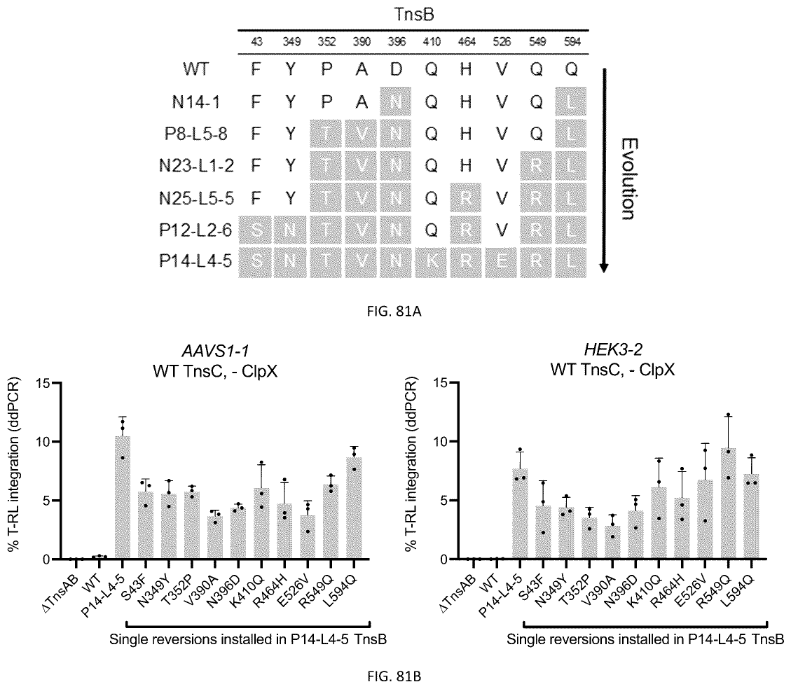

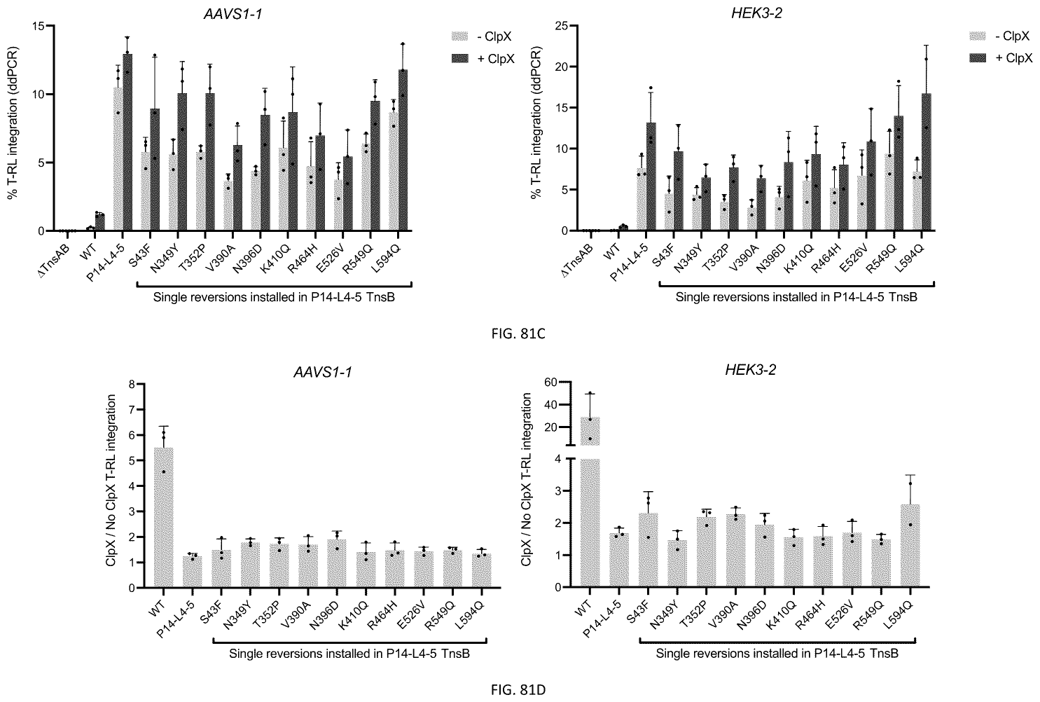

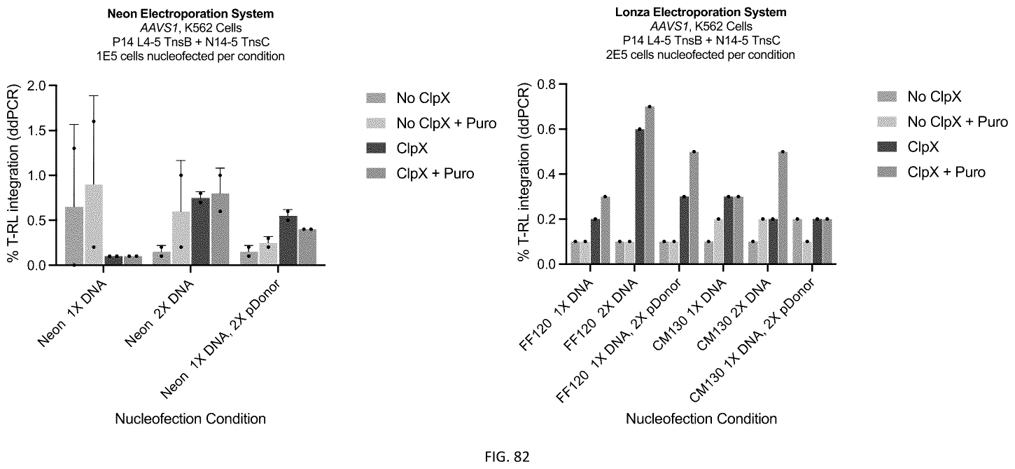

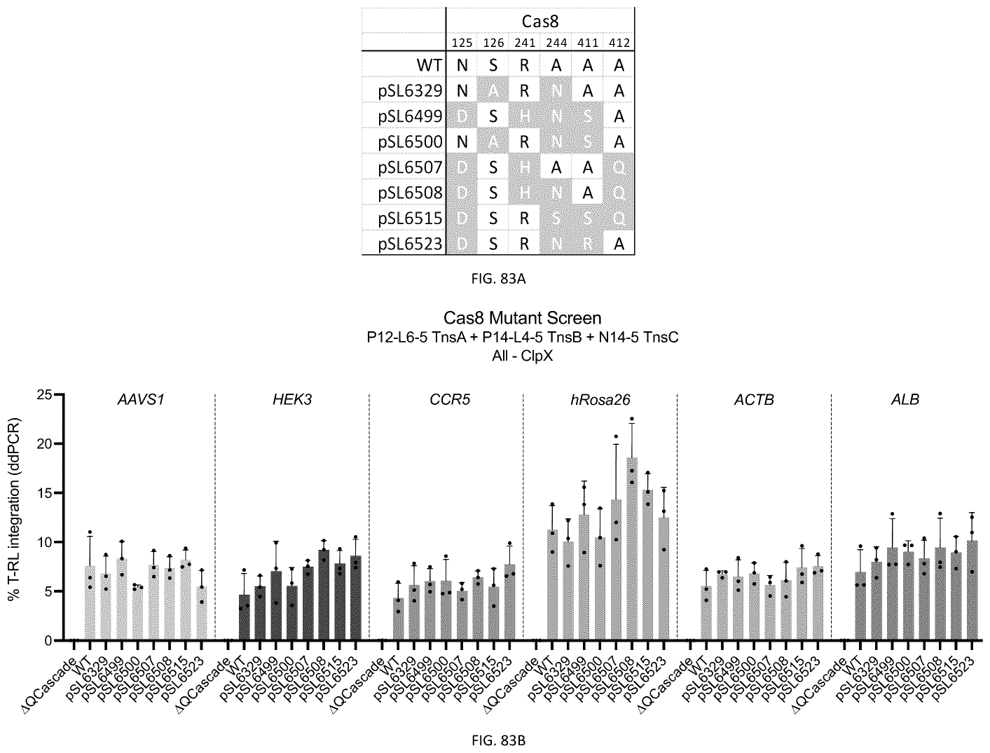

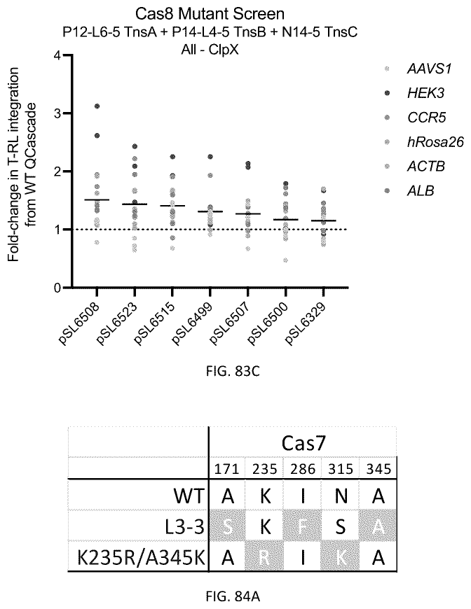

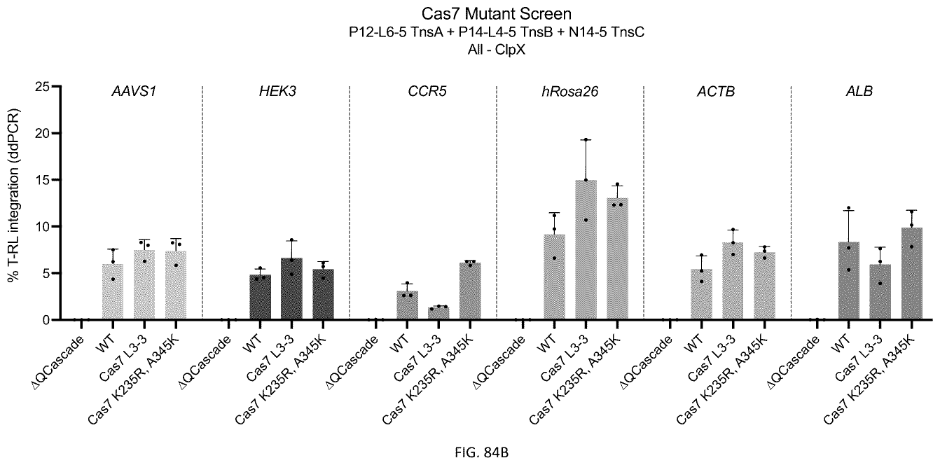

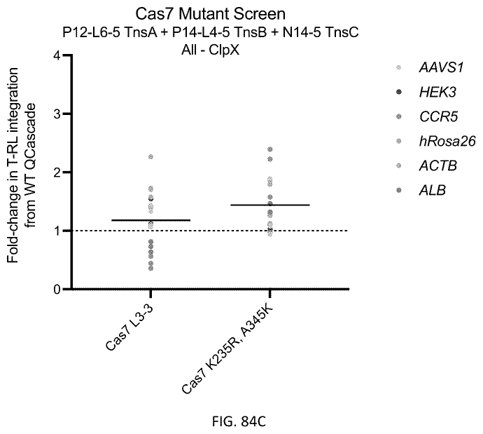

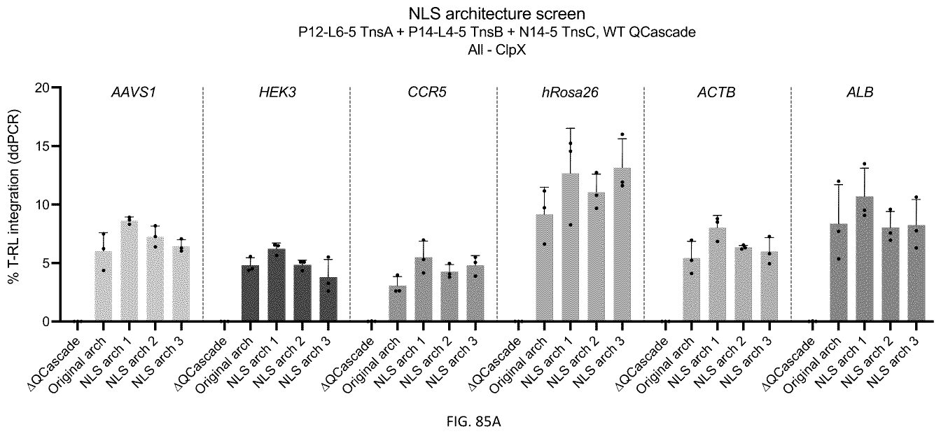

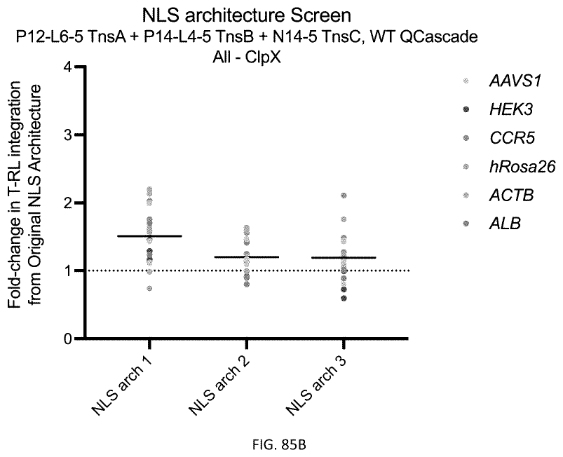

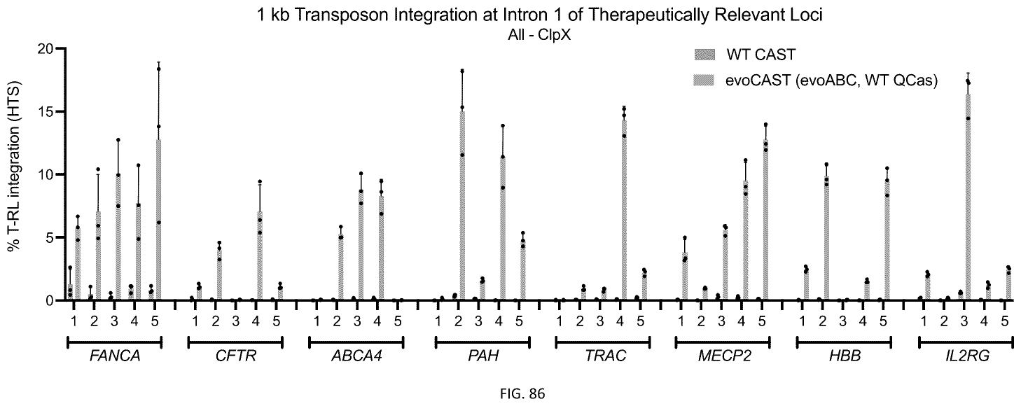

Variants from later stages of PANCE N23 and PACE P9 did not have improved mammalian activity. The activity of variants from earlier in the PANCE N23 and PACE P9 evolutionary trajectories was revisited. These earlier variants demonstrated improved activity in HEK293 Ts (FIG. 8). The three top performers identified are N23-P16-L1-2, N23-P16-L1-5, and P9-48h-L4-5.

### Example 3

### Evolution of QCascade INTEGRATE from Tn6677 and Tn7016

Circuits for multiple rounds of PACE and PANCE have been verified for Tn6677 and Tn7016. Characterization of evolved variants is completed using similar integration assays as described for TnsABC above.

TABLE 1

| Amino Acid of | | Amino Acid of | | Amino Acid of | |
|---|---|---|---|---|---|
| wild-type TnsA | Amino Acid | WT TnsB (SEQ | Amino Acid | WT TnsC (SEQ | Amino Acid |
| (SEQ ID NO: 1) | Modification | ID NO: 2) | Modification | ID NO: 3) | Modification |
| A2 | A2T | A2 | A2S | I9 | I9V |
| T3 | T3I | A2 | A2T | A15 | A15V |
| L5 | L5S | G5 | G5R | F16 | F16Y |
| T28 | T28A | S22 | S22P | S18 | S18F |
| A57 | A57T | E24 | E24D | S21 | S21N |
| F77 | F77L | L25 | L25I | N64 | N64D |
| Y80 | Y80D | A29 | A29S | H81 | H81Y |
| K107 | K107M | P75 | P75T | D86 | D86Y |
| K107 | K107R | I141 | I141T | N87 | N87K |
| Y110 | Y110C | V199 | V199I | V99 | V99I |
| Y110 | Y110D | S215 | S215R | E109 | E109D |
| D116 | D116G | D319 | D319V | E142 | E142K |
| E122 | E122A | Y347 | Y347F | E142/A216 | E142K/A216S |
| D142 | D142E | S364 | S364N | V147 | V147I |
| M155 | M155I | E370 | E370K | N153 | N153D |
| K161 | K161R | N383 | N383D | I168 | I168M |
| N166 | N166D | V439 | V439A | A180 | A180E |
| K173 | K173E | E454 | E454D | A216 | A216S |
| Y177 | Y177N | E454 | E454G | L230 | L230F |
| Y177 | Y177D | S458 | S458N | K285 | K285E |
| C185 | C185R | V485 | V485F | R304 | R304R |
| D211 | D211Y | R509 | R509G | | |
| K216 | K216E | D533 | D533A | | |
| A227 | A227P | A538 | A538V | | |
| G230 | G230D | H565 | H565Y | | |
| G230 | G230S | A581 | A581T | | |
| A2/G230 | A2T/G230D | H586 | H586L | | |
| K107/N166 | K107M/N166D | N595 | N595K | | |
| K107/N166/T2 | K107M/N166D/ | D596 | D596N | | |
| | T2A | | | | |
| K107/N166/ | K107M/N166D/ | D597 | D597N | | |
| P227 | P227A | | | | |
| K107/N166/ | K107M/N166D/ | D597 | D597Y | | |
| P227/T2 | T2A/P227A | | | | |
| D211/Y110 | D211Y/Y110C | I600 | I600V | | |
| D211/D142 | D211Y/D142E | D597/A2 | D597N/A2T | | |
| Y110/M155/ | Y110D/M155I/ | E24/L25 | E24D/L25I | | |
| G230 | G230S | | | | |
| E122/M155 | E122A/M155I | E24/L25/H565/ | E24D/L25I/ | | |
| | | R509/S458/I600 | H565Y/R509G/ | | |
| | | | S458N/I600V | | |
| M155/Y177 | M155I/Y177N | P75/D597 | P75T/D597N | | |
| M155/Y177 | M155I/Y177D | I141/E454/D533/ | I141T/E454G/ | | |
| | | N595 | D533A/N595K | | |
| | | A581/E370/ | A581T/E370K/ | | |
| | | E454 | E454D | | |
| | | E370/A581 | E370K/A581T | | |
| | | E370/E454 | E370K/E454D | | |
| | | R509/S458 | R509G/S458N | | |
| | | H565/R509/ | H565Y/R509G/ | | |
| | | S458 | S458N | | |
| | | H565/R509/ | H565Y/R509G/ | | |
| | | S458/I600 | S458N/I600V | | |
| | | H565/H586/ | H565Y/H586L/ | | |
| | | D596 | D596N | | |
| | | H565/R509/ | H565Y/R509G/ | | |
| | | S458/I600/E24 | S458N/I600V/ | | |
| | | | E24D | | |
| | | H565/R509/ | H565Y/R509G/ | | |
| | | S458/I600/L25 | S458N/I600V/ | | |
| | | | L25I | | |
| | | H565/R509/ | H565Y/R509G/ | | |
| | | S458/I600/A29 | S458N/I600V/ | | |
| | | | A29S | | |
| | | H565/R509/ | H565Y/R509G/ | | |
| | | S458/I600/S215 | S458N/I600V/ | | |
| | | | S215R | | |
| | | H565/R509/ | H565Y/R509G/ | | |
| | | S458/I600/D319 | S458N/I600V/ | | |
| | | | D319V | | |
| | | H565/R509/ | H565Y/R509G/ | | |
| | | S458/I600/S364 | S458N/I600V/ | | |
| | | | S364N | | |
| | | H565/R509/ | H565Y/R509G/ | | |
| | | S458/I600/N383 | S458N/I600V/ | | |
| | | | N383D | | |
| | | H565/R509/ | H565Y/R509G/ | | |
| | | S458/I600/H586 | S458N/I600V/ | | |
| | | | H586L | | |

TABLE 2

| Amino Acid of | | Amino Acid of | | Amino Acid of | |
|---|---|---|---|---|---|
| wild-type TnsA | Amino Acid | WT TnsB (SEQ | Amino Acid | WT TnsC (SEQ | Amino Acid |
| (SEQ ID NO: 4) | Modification | ID NO: 5) | Modification | ID NO: 6) | Modification |
| R4 | R4K | M1 | M1V | M1 | M1L |
| N5 | N5K | M1 | M1I | M1 | M1V |
| P9 | P9S | M1 | M1L | N2 | N2S |
| A10 | A10P | T2 | T2I | N2/K67/A95/ | N2S/K67N/ |
| | | | | V226 | A95D/V226E |
| N12 | N12D | T2 | T2A | A3 | A3T |
| T21 | T21I | F4 | F4L | T5 | T5P |
| V23 | V23M | F4/Y23/A590 | F4L/Y23H/ | T5 | T5A |
| | | | A590S | | |
| S25 | S25N | F5 | F5L | T5 | T5S |
| S25 | S25R | F8 | F8L | E6 | E6D |
| V26 | V26M | F8 | F8V | E6/N316 | E6D/N316D |
| V26 | V26G | F8 | F8S | I7 | I7S |
| S31 | S31N | D9 | D9N | I7 | I7V |
| S32 | S32I | E10 | E10K | I9 | I9F |
| E34 | E34A | E10 | E10D | Q11 | Q11R |
| F35 | F35L | S11 | S11I | L12 | L12M |
| A37 | A37D | S11 | S11R | N14 | N14D |
| H41 | H41L | S11 | S11G | N14 | N14S |
| D45 | D45N | S11/S55/N120/ | S11G/S55A/ | M21 | M21I |
| | | I362/K584/ | N120K/I362V/ | | |
| | | D600/D604 | K584R/D600G/ | | |
| | | | D604N | | |
| I47 | I47V | L12 | L12P | H22 | H22P |
| I47/P88/I147 | I47V/P88T/ | V13 | V13M | H22 | H22Y |
| | I147V | | | | |
| E48 | E48G | V13 | V13G | K26 | K26N |
| G51 | G51V | V13 | V13E | K26 | K26R |
| S52 | S52I | V13 | V13L | T27 | T27I |
| E55 | E55K | P14 | P14L | M31 | M31I |
| E55 | E55D | L15 | L15Q | L35 | L35R |
| E60 | E60K | K16 | K16N | N38 | N38S |
| F61 | F61L | K16 | K16R | N38/A95/E303 | N38S/A95D/ |
| | | | | | E303D |
| S65 | S65T | P17 | P17T | S43 | S43P |
| S65 | S65A | P17 | P17L | D44 | D44N |
| P67 | P67T | P17 | P17S | D44 | D44G |
| P67 | P67L | T19 | T19I | D44/K118 | D44G/K118R |
| P67 | P67S | T19 | T19S | Q46 | Q46L |
| P67 | P67H | T19 | T19A | C47 | C47S |
| T69 | T69A | T19 | T19P | T54 | T54I |
| A72 | A72V | T19/I169/Q549 | T19P/I169L/ | S59 | S59T |
| | | | Q549K | | |
| A72 | A72D | P20 | P20S | H60 | H60Y |
| S75 | S75I | P20 | P20L | T61 | T61A |
| S75 | S75R | T21 | T21A | H64 | H64Y |
| S75 | S75T | Q22 | Q22R | Y65 | Y65H |
| K79 | K79E | Y23 | Y23H | K67 | K67N |
| T80 | T80P | V24 | V24M | K67/A95/V226 | K67N/A95D/ |
| | | | | | V226E |
| K82 | K82E | K25 | K25R | K67 | K67R |
| K87 | K87R | L26 | L26M | R68 | R68Q |
| P88 | P88L | D27 | D27A | A71 | A71G |
| P88 | P88T | D27 | D27G | T72 | T72A |
| P88 | P88A | D28 | D28N | N74 | N74D |
| P88/I147 | P88T/I147V | D28 | D28Y | S76 | S76C |
| P88/I147/F154 | P88T/I147V/ | A29 | A29T | S76 | S76Y |
| | F154C | | | | |
| P88/I147/V170/ | P88T/I147V/ | A29 | A29V | T79 | T79I |
| F182 | V170L/F182L | | | | |
| P88/I147/V170/ | P88T/I147V/ | N30 | N30K | M80 | M80I |
| F182/G51 | V170L/F182L/ | | | | |
| | G51V | | | | |
| P88/I147/V170/ | P88T/I147V/ | I32 | I32F | P81 | P81S |
| F180/F182 | V170L/F180L/ | | | | |
| | F182L | | | | |
| P88/I128/ | P88T/I128V/ | I32 | I32S | V84 | V84L |
| I147/V170/ | I147V/ | | | | |
| F182 | V170L/F182L | | | | |
| S90 | S90F | Q33 | Q33H | R89 | R89L |
| A93 | A93S | L36 | L36M | A95 | A95D |
| K91 | K91N | D37 | D37A | A95 | A95T |
| K91 | K91E | D37 | D37Y | A102 | A102T |
| A93 | A93T | F39 | F39L | E105 | E105D |
| S94 | S94N | S40 | S40P | E105 | E105K |
| L96 | L96P | D41 | D41E | S109 | S109N |
| R98 | R98Q | T42 | T42I | S109 | S109R |
| A99 | A99D | T42 | T42A | S110 | S110P |
| A99 | A99V | T42 | T42K | Q111 | Q111R |
| E100 | E100K | F43 | F43L | I112 | I112T |
| A103 | A103T | F43 | F43S | K113 | K113N |
| A106 | A106T | F43 | F43V | K113 | K113E |
| S108 | S108A | F43/A415 | F43L/A415V | K114 | K114N |
| S108/I47 | S108A/I47V | F43/Y349 | F43S/Y349N | K114 | K114E |
| S108/T208 | S108A/T208I | F43/V84/I144/ | F43S/V84A/ | K114 | K114M |
| | | Y349/K517 | I144V/Y349N/ | | |
| | | | K517M | | |
| I113 | I113F | K44 | K44N | G116 | G116D |
| V116 | V116F | N45 | N45D | K118 | K118N |
| V116 | V116I | N45 | N45S | K118 | K118R |
| V125 | V125M | Q49 | Q49R | K118/A1201 | K118R/A1201V |
| V125 | V125A | K52 | K52Q | T119 | T119I |
| N126 | N126T | S55 | S55A | D120 | D120V |
| I128 | I128V | T56 | T56A | K123 | K123N |
| I128 | I128L | D58 | D58E | L129 | L129M |
| L129 | L129P | K60 | K60Q | I130 | I130V |
| L135 | L135M | S62 | S62T | I130/N234/E303 | I130V/N234H/ |
| | | | | | E303D |
| S139 | S139N | R63 | R63K | K131 | K131R |
| S139 | S139G | R63 | R63G | A132 | A132S |
| G143 | G143C | Q67 | Q67R | K134 | K134M |
| G143 | G143V | Q67 | Q67H | K134 | K134N |
| G146 | G146D | Q67 | Q67K | F142 | F142V |
| G146 | G146S | D71 | D71Y | L145 | L145M |
| I147 | I147V | K74 | K74R | I146 | I146T |
| K149 | K149E | E76 | E76K | E147 | E147K |
| K149 | K149R | F78 | F78C | F148 | F148S |
| K149 | K149T | K79 | K79R | S150 | S150F |
| S153 | S153I | G80 | G80V | R154 | R154K |
| S153 | S153R | G80 | G80D | R154/E269 | R154K/E269D |
| S153 | S153N | G80/V593 | G80D/V593M | Q155 | Q155H |
| F154 | F154C | G80/V593/I144/ | G80D/V593M/ | E166 | E166D |
| | | D606 | I144V/D606A | | |
| H156 | H156R | G80/V593/D606/ | G80D/V593M/ | K169 | K169E |
| | | T42/M1 | D606A/T42I/ | | |
| | | | M1V | | |
| H156 | H156L | G80/V593/D606/ | G80D/V593M/ | P178 | P178S |
| | | T42 | D606A/T42I | | |
| S158 | S158N | G81 | G81S | A180 | A180V |
| S158 | S158R | G81 | G81V | A181 | A181T |
| G159 | G159V | G81 | G81D | A181 | A181S |
| V160 | V160A | D82 | D82N | I183 | I183V |
| K162 | K162R | V83 | V83G | A184 | A184S |
| N164 | N164D | V83 | V83M | A184 | A184T |
| I166 | I166L | V83 | V83A | A184 | A184V |
| S167 | S167I | V84 | V84A | P187 | P187S |
| S168 | S168I | V84 | V84G | A190 | A190V |
| S168 | S168R | R85 | R85G | A190 | A190T |
| S168 | S168N | R85 | R85K | V194 | V194M |
| Q169 | Q169R | P86 | P86L | V194 | V194A |
| V170 | V170M | N87 | N87S | R197 | R197I |
| V170 | V170G | W88 | W88* | Y201 | Y201N |
| V170 | V170L | R89 | R89C | L204 | L204M |
| V170/A207 | V170M/A207T | V91 | V91G | D207 | D207N |
| V170/A207/ | V170M/A207T/ | V91 | V91A | K209 | K209N |
| S108 | S108A | | | | |
| T177 | T177I | A92 | A92V | Q213 | Q213H |
| T177 | T177A | A92 | A92T | Q213 | Q213V |
| S179 | S179R | R95 | R95K | A219 | A219S |
| F180 | F180C | K97 | K97R | K221 | K221N |
| F180 | F180L | E100 | E100D | K221/D44 | K221N/D44N |
| F182 | F182C | S101 | S101A | D225 | D225N |
| F182 | F182L | D104 | D104V | V226 | V226E |
| G183 | G183S | A106 | A106D | P227 | P227T |
| M185 | M185I | A106 | A106T | K229 | K229E |
| K187 | K187R | D110 | D110N | S232 | S232N |
| G188 | G188D | N112 | N112H | K233 | K233R |
| V190 | V190I | H113 | H113Y | K233 | K233N |
| K191 | K191N | M115 | M115R | N234 | N234H |
| A192 | A192S | N117 | N117Y | T236 | T236A |
| D193 | D193N | T119 | T119A | A238 | A238V |
| G195 | G195V | N120 | N120D | A238 | A238S |
| G195 | G195D | N120 | N120K | A241 | A241S |
| G195 | G195S | N120 | N120S | E246 | E246D |
| C196 | C196W | G124 | G124V | K251 | K251N |
| T200 | T200A | D125 | D125N | H252 | H252Y |
| T204 | T204I | D125 | D125E | H252 | H252R |
| A207 | A207V | K127 | K127R | E256 | E256D |
| A207 | A207T | F129 | F129L | A257 | A257S |
| T208 | T208I | D130 | D130N | A261 | A261V |
| | | K131 | K131M | S263 | S263I |
| | | E134 | E134D | S263 | S263N |
| | | E134 | E134G | N265 | N265D |
| | | A139 | A139S | Y267 | Y267C |
| | | A139 | A139T | E269 | E269K |
| | | P142 | P142S | E269 | E269D |
| | | I144 | I144V | K271 | K271E |
| | | A145 | A145S | K271 | K271R |
| | | A145 | A145T | H272 | H272Y |
| | | T146 | T146A | I274 | I274V |
| | | A147 | A147V | F280 | F280L |
| | | Q149 | Q149R | F280/S340 | F280L/S340L |
| | | Y150 | Y150H | D281 | D281N |
| | | I155 | I155L | D281 | D281G |
| | | V156 | V156A | K285 | K285G |
| | | V156 | V156L | K286 | K286N |
| | | V156 | V156M | K288 | K288R |
| | | V156/D604 | V156M/D604G | S291 | S291F |
| | | I157 | K157V | S291 | S291P |
| | | E158 | E158A | K292 | K292N |
| | | N159 | N159S | K296 | K296R |
| | | V163 | V163G | K296 | K296N |
| | | E164 | E164A | I299 | I299S |
| | | E164 | E164G | D301 | D301G |
| | | E164 | E164D | E303 | E303D |
| | | E164/G165 | E164G/G165D | I304 | I304T |
| | | E164/N173 | E164G/N173T | I304 | I304V |
| | | G165 | G165D | E306 | E306G |
| | | I167 | I167V | V307 | V307L |
| | | I169 | I169L | V307 | V307G |
| | | I169 | I169T | V307 | V307A |
| | | N173 | N173S | V307 | V307D |
| | | N173 | N173T | V307 | V307G |
| | | N173 | N173H | I308 | I308N |
| | | A174 | A174S | N310 | N310S |
| | | A174 | A174T | Y313 | Y313H |
| | | N176 | N176D | N314 | N314K |
| | | A181 | A181S | N316 | N316K |
| | | I182 | I182L | N316 | N316D |
| | | I182 | I182V | A317 | A317D |
| | | I182 | I182T | L318 | L318Q |
| | | A186 | A186E | D319 | D319N |
| | | A186 | A186T | P320 | P320S |
| | | V187 | V187G | P320 | P320L |
| | | V187 | V187A | M323 | M323I |
| | | A190 | A190T | L324 | L324M |
| | | A190 | A190S | D326 | D326N |
| | | F195 | F195S | V328 | V328M |
| | | A197 | A197P | V328 | V328A |
| | | D198 | D198G | A330 | A330D |
| | | D198 | D198N | I331 | I331V |
| | | A205 | A205S | V332 | V332G |
| | | V208 | V208M | S340 | S340L |
| | | P209 | P209T | T341 | T341A |
| | | T211 | T211I | A343 | A343G |
| | | E215 | E215D | S344 | S344N |
| | | E218 | E218D | I355 | I355V |
| | | P223 | P223S | F412 | F412V |
| | | P223 | P223H | V418 | V418F |
| | | L226 | L226V | Y427 | Y427C |
| | | I227 | I227V | R514 | R514K |
| | | D231 | D231N | S1198 | S1198L |
| | | E232 | E232K | A1201 | A1201V |
| | | I235 | I235V | G1206 | G1206S |
| | | I235 | I235T | C1212 | C1212G |
| | | R239 | R239G | F1260 | F1260L |
| | | I246 | I246V | V1282 | V1282M |
| | | V248 | V248E | S76/D44/K118 | S76Y/D44N/ |
| | | | | | K118R |
| | | V248 | V248M | S76/D44/K118 | S76Y/D44G/ |
| | | | | | K118R |
| | | S250 | S250I | S76/D44/I130/ | S76Y/D44N/ |
| | | | | N234/E303 | I130V/N234H/ |
| | | | | | E303D |
| | | S259 | S259N | S76/D44/I130/ | S76Y/D44G/ |
| | | | | N234/E303 | I130V/N234H/ |
| | | | | | E303D |
| | | Y260 | Y260C | K118/A1201/ | K118R/A1201V/ |
| | | | | D44 | D44G |
| | | K261 | K261R | K118/A1201/ | K118R/A1201V/ |
| | | | | S76 | S76Y |
| | | S262 | S262N | K118/A1201/ | K118R/A1201V/ |
| | | | | D44/S76 | D44G/S76Y |
| | | P263 | P263L | R197/N314 | R197I/N314K |
| | | S267 | S267N | S76/A181/V194 | S76Y/A181S/ |
| | | | | | V194M |
| | | A269 | A269V | S76/K118/H252/ | S76Y/K118R/ |
| | | | | K292 | H252R/K292N |
| | | T273 | T273I | S76/I274 | S76Y/I274V |
| | | T273 | T273N | S76/A102/K118/ | S76Y/A102T/ |
| | | | | V307 | K118R/V307G |
| - | | H274 | H274Y | L12/S76 | L12M/S76Y |
| | | K277 | K277N | K67/A95/V226 | K67N/A95D/ |
| | | | | | V226E |
| | | K277 | K277R | K26/S76 | K26N/S76Y |
| | | P278 | P278S | H22/S76/D319 | H22Y/S76Y/ |
| | | | | | D319N |
| | | S280 | S280T | R154/E269 | R154K/E269D |
| | | L281 | L281M | S76/A238 | S76Y/A238S |
| | | D282 | D282E | S76/S263 | S76Y/S263N |
| | | D282 | D282N | S59/S76/E306/ | S59T/S76Y/ |
| | | | | N316 | E306G/N316D |
| | | A283 | A283T | S76/L12 | S76Y/L12M |
| | | A283 | A283S | S76/I7 | S76Y/I7V |
| | | A283/Y349/ | A283T/Y349H/ | S76/A238/K296/ | S76Y/A238S/ |
| | | K365 | K365R | V328 | K296N/V328M |
| | | A283/Y349/ | A283T/Y349H/ | | |
| | | K365/D396/ | K365R/D396N/ | | |
| | | Q594 | Q594L | | |
| | | A283/Y349/ | A283T/Y349H/ | | |
| | | P352/K365/ | P352S/K365R/ | | |
| | | D396/Q594/ | D396N/Q594L/ | | |
| | | H596/K131 | H596L/K131M | | |
| | | N285 | N285S | | |
| | | E287 | E287D | | |
| | | L288 | L288M | | |
| | | N290 | N290K | | |
| | | F295 | F295S | | |
| | | F298 | F298I | | |
| | | F298 | F298S | | |
| | | V302 | V302I | | |
| | | V303 | V303M | | |
| | | A307 | A307S | | |
| | | N313 | N313S | | |
| | | H316 | H316R | | |
| | | A317 | A317V | | |
| | | S320 | S320N | | |
| | | S320 | S320R | | |
| | | I323 | I323L | | |
| | | I325 | I325V | | |
| | | R331 | R331K | | |
| | | K332 | K332E | | |
| | | I339 | I339V | | |
| | | V345 | V345L | | |
| | | V345 | V345M | | |
| | | E348 | E348K | | |
| | | Y349 | Y349H | | |
| | | Y349 | Y349D | | |
| | | Y349 | Y349N | | |
| | | Y349 | Y349C | | |
| | | P352 | P352S | | |
| | | P352 | P352T | | |
| | | P352/A390 | P352T/A390V | | |
| | | E353 | E353Q | | |
| | | E353 | E353D | | |
| | | L354 | L354M | | |
| | | G356 | G356S | | |
| | | N361 | N361D | | |
| | | I362 | I362V | | |
| | | I362 | I362T | | |
| | | I362/F446 | I362T/F446I | | |
| | | L363 | L363P | | |
| | | L363 | L363T | | |
| | | L363 | L363M | | |
| | | E364 | E364G | | |
| | | K365 | K365R | | |
| | | E366 | E366G | | |
| | | E367 | E367G | | |
| | | K369 | K369N | | |
| | | K369 | K369E | | |
| | | K369 | K369M | | |
| | | P370 | P370S | | |
| | | E371 | E371K | | |
| | | V372 | V372M | | |
| | | D373 | D373G | | |
| | | I375 | I375V | | |
| | | M376 | M376I | | |
| | | T380 | T380P | | |
| | | T380 | T380A | | |
| | | E383 | E383K | | |
| | | E383 | E383D | | |
| | | F385 | F385L | | |
| | | H386 | H386Y | | |
| | | I389 | I389V | | |
| | | A390 | A390V | | |
| | | A390 | A390I | | |
| | | A390/D396/ | A390V/D396N/ | | |
| | | Q594 | Q594L | | |
| | | V392 | V392I | | |
| | | D396 | D396N | | |
| | | D396 | D396G | | |
| | | D396 | D396K | | |
| | | D396/Q594 | D396N/Q594L | | |
| | | S397 | S397P | | |
| | | S399 | S399N | | |
| | | S399 | S399G | | |
| | | T402 | T402I | | |
| | | R403 | R403G | | |
| | | R403 | R403I | | |
| | | R403 | R403K | | |
| | | R403 | R403S | | |
| | | I404 | I404T | | |
| | | I404 | I404V | | |
| | | K407 | K407R | | |
| | | K407 | K407E | | |
| | | R408 | R408K | | |
| | | Q410 | Q410K | | |
| | | Q410 | Q410H | | |
| | | Q410 | Q410R | | |
| | | Q411 | Q411H | | |
| | | G412 | G412V | | |
| | | F413 | F413L | | |
| | | D414 | D414N | | |
| | | A415 | A415V | | |
| | | A415 | A415T | | |
| | | A415/T502 | A415V/T502I | | |
| | | Y416 | Y416C | | |
| | | M421 | M421I | | |
| | | N422 | N422K | | |
| | | E423 | E423K | | |
| | | E423 | E423D | | |
| | | E424 | E424A | | |
| | | E425 | E425K | | |
| | | E426 | E426D | | |
| | | T427 | T427A | | |
| | | T427 | T427S | | |
| | | R428 | R428K | | |
| | | F429 | F429L | | |
| | | S430 | S430A | | |
| | | M431 | M431L | | |
| | | R434 | R434H | | |
| | | R434 | R434C | | |
| | | R434 | R434S | | |
| | | I435 | I435V | | |
| | | D437 | D437G | | |
| | | D437 | D437N | | |
| | | T440 | T440S | | |
| | | T440 | T440I | | |
| | | R443 | R443C | | |
| | | G445 | G445S | | |
| | | F446 | F446L | | |
| | | F446 | F446I | | |
| | | Y448 | Y448C | | |
| | | E450 | E450D | | |
| | | E450 | E450* | | |
| | | E450 | E450G | | |
| | | M452 | M452I | | |
| | | T456 | T456P | | |
| | | T456/T502 | T456P/T502I | | |
| | | T456 | T456A | | |
| | | T456 | T456I | | |
| | | A459 | A459T | | |
| | | D460 | D460N | | |
| | | K463 | K463N | | |
| | | H464 | H464N | | |
| | | H464/T502 | H464N/T502I | | |
| | | H464 | H464R | | |
| | | H464/P17 | H464R/P17T | | |
| | | H464 | H464S | | |
| | | E470 | E470K | | |
| | | V472 | V472M | | |
| | | V472 | V472A | | |
| | | K473 | K473D | | |
| | | K473 | K473N | | |
| | | E494 | E494D | | |
| | | E494 | E494G | | |
| | | S495 | S495A | | |
| | | E498 | E498A | | |
| | | E498 | E498K | | |
| | | C501 | C501Y | | |
| | | T502 | T502I | | |
| | | T502 | T502S | | |
| | | P504 | P504S | | |
| | | P504 | P504L | | |
| | | T505 | T505A | | |
| | | G506 | G506Y | | |
| | | G506 | G506D | | |
| | | G506 | G506L | | |
| | | G506 | G506S | | |
| | | T508 | T508A | | |
| | | D509 | D509Y | | |
| | | D509 | D509E | | |
| | | C510 | C510Y | | |
| | | S512 | S512N | | |
| | | I513 | I513L | | |
| | | I513 | I513V | | |
| | | I513 | I513F | | |
| | | Y514 | Y514H | | |
| | | K517 | K517M | | |
| | | K517 | K517N | | |
| | | K517 | K517Q | | |
| | | K520 | K520R | | |
| | | K521 | K521N | | |
| | | I522 | I522T | | |
| | | I522 | I522V | | |
| | | I522 | I522F | | |
| | | E525 | E525K | | |
| | | V526 | V526E | | |
| | | V526 | V526M | | |
| | | I527 | I527V | | |
| | | S530 | S530N | | |
| | | S530 | S530R | | |
| | | K531 | K531T | | |
| | | D532 | D532G | | |
| | | D532 | D532Y | | |
| | | S533 | S533Y | | |
| | | G535 | G535D | | |
| | | A537 | A537T | | |
| | | K538 | K538R | | |
| | | K538 | K538N | | |
| | | R540 | R540K | | |
| | | R540 | R540G | | |
| | | M541 | M541L | | |
| | | A542 | A542T | | |
| | | I543 | I543L | | |
| | | H544 | H544R | | |
| | | E545 | E545A | | |
| | | R546 | R546G | | |
| | | R546 | R546K | | |
| | | V547 | V547M | | |
| | | K548 | K548Q | | |
| | | K548 | K548R | | |
| | | Q549 | Q549K | | |
| | | Q549 | Q549R | | |
| | | E550 | E550A | | |
| | | Q551 | Q551K | | |
| | | E552 | E552D | | |
| | | E552 | E552K | | |
| | | V553 | V553I | | |
| | | F554 | F554V | | |
| | | E556 | E556K | | |
| | | E556 | E556G | | |
| | | S557 | S557A | | |
| | | K558 | K558R | | |
| | | T559 | T559P | | |
| | | T559 | T559I | | |
| | | T559 | T559A | | |
| | | K560 | K560R | | |
| | | A561 | A561T | | |
| | | A561 | A561G | | |
| | | K562 | K562R | | |
| | | K562 | K562N | | |
| | | I563 | I563L | | |
| | | T564 | T564I | | |
| | | A565 | A565S | | |
| | | A565 | A565V | | |
| | | K567 | K567R | | |
| | | K568 | K568N | | |
| | | K568 | K568R | | |
| | | Q569 | Q569K | | |
| | | Q569 | Q569L | | |
| | | Q569 | Q569R | | |
| | | A570 | A570V | | |
| | | Q571 | Q571R | | |
| | | D574 | D574N | | |
| | | V575 | V575M | | |
| | | V575 | V575A | | |
| | | S576 | S576R | | |
| | | T580 | T580I | | |
| | | T580 | T580A | | |
| | | T582 | T582I | | |
| | | T582 | T582S | | |
| | | I583 | I583V | | |
| | | K584 | K584R | | |
| | | V585 | V585M | | |
| | | S586 | S586P | | |
| | | S586 | S586A | | |
| | | S586 | S586F | | |
| | | E587 | E587A | | |
| | | E588 | E588K | | |
| | | E588 | E588G | | |
| | | E588 | E588D | | |
| | | S589 | S589I | | |
| | | S589 | S589R | | |
| | | S589 | S589N | | |
| | | A590 | A590S | | |
| | | A590 | A590T | | |
| | | A591 | A591V | | |
| | | P592 | P592L | | |
| | | V593 | V593M | | |
| | | V593 | V593A | | |
| | | Q594 | Q594L | | |
| | | K595 | K595R | | |
| | | K595 | K595N | | |
| | | H596 | H596Y | | |
| | | H596 | H596L | | |
| | | H596 | H596P | | |
| | | H596/H464/ | H596L/H464R/ | | |
| | | I235/P17 | I235V/P17T | | |
| | | I597 | I597T | | |
| | | I597 | I597V | | |
| | | N599 | N599H | | |
| | | D600 | D600L | | |
| | | D600 | D600N | | |
| | | D600 | D600G | | |
| | | D600 | D600V | | |
| | | N601 | N601S | | |
| | | N601 | N601K | | |
| | | S602 | S602A | | |
| | | S602 | S602P | | |
| | | S602 | S602Y | | |
| | | D603 | D603A | | |
| | | D603 | D603V | | |
| | | D604 | D604G | | |
| | | D604 | D604Y | | |
| | | D604 | D604N | | |
| | | D606 | D606A | | |
| | | D606 | D606V | | |
| | | D606 | D606Y | | |
| | | D606/T456/ | D606V/T456A/ | | |
| | | D396/P352/ | D396K/P352T/ | | |
| | | I235 | I235T | | |
| | | D607 | D607Y | | |
| | | D607 | D607E | | |
| | | D608 | D608N | | |
| | | A611 | A611T | | |
| | | E613 | E613D | | |
| | | R618 | R618I | | |
| | | T620 | T620P | | |
| | | A656 | A656V | | |
| | | A415/T456/ | A415V/T456P/ | | |
| | | T502 | T502I | | |
| | | T456/T502/ | T456P/T502I/ | | |
| | | Q549 | Q549K | | |
| | | I169/T456/T502/ | I169L/T456P/ | | |
| | | Q549 | T502I/Q549K | | |
| | | G80/T456/T502/ | G80D/T456P/ | | |
| | | V593/D606 | T502I/V593M/ | | |
| | | | D606A | | |
| | | M1/T42/G80/ | M1V/T42I/G80D/ | | |
| | | T456/T502/V593/ | T456P/T502I/ | | |
| | | D606 | V593M/D606A | | |
| | | G80/I144/T456/ | G80D/I144V/ | | |
| | | T502/V593/ | T456P/T502I/ | | |
| | | D606 | V593M/D606A | | |
| | | T19/I169/T456/ | T19P/I169/ | | |
| | | T502/Q549 | T456P/T502I/ | | |
| | | | Q549K | | |
| | | F43/A415/T456/ | F43L/A415V/ | | |
| | | T502 | T456P/T502I | | |
| | | P352/A390/ | P352T/A390V/ | | |
| | | D396/Q594 | D396N/Q594L | | |
| | | P352/A390/ | P352T/A390V/ | | |
| | | D396/Q549/ | D396N/ | | |
| | | Q594 | Q549R/Q594L | | |
| | | P352/A390/ | P352T/A390V/ | | |
| | | D396/H464/ | D396N/H464R/ | | |
| | | Q549/Q594 | Q549R/Q594L | | |
| | | F43/Y349/P352/ | F43S/Y349N/ | | |
| | | A390/ | P352T/A390V/ | | |
| | | D396/H464/ | D396N/H464R/ | | |
| | | Q549/Q594 | Q549R/Q594L | | |
| | | F43/P352/A390/ | F43S/P352T/ | | |
| | | D396/H464/ | A390V/ | | |
| | | Q549/Q594 | D396N/H464R/ | | |
| | | | Q549R/Q594L | | |
| | | F43/Y349/P352/ | F43S/Y349D/ | | |
| | | A390/ | P352T/A390V/ | | |
| | | D396/H464/ | D396N/H464R/ | | |
| | | Q549/Q594 | Q549R/Q594L | | |
| | | F43/Y349/P352/ | F43S/Y349D/ | | |
| | | A390/ | P352T/A390V/ | | |
| | | D396/H464/ | D396N/H464R/ | | |
| | | V526/Q549/ | V526E/Q549R/ | | |
| | | Q594 | Q594L | | |
| | | P352/A390/ | P352T/A390V/ | | |
| | | D396/Q549/ | D396N/ | | |
| | | S586/Q594 | Q549R/S586A/ | | |
| | | | Q594L | | |
| | | R63/E158/P352/ | R63G/E158A/ | | |
| | | A390/ | P352T/A390V/ | | |
| | | D396/Q549/ | D396N/ | | |
| | | S586/Q594 | Q549R/S586A/ | | |
| | | | Q594L | | |
| | | E164/G165/ | E164G/G165D/ | | |
| | | P352/L363/ | P352T/L363P/ | | |
| | | A390/D396/ | A390V/D396N/ | | |
| | | Q410/Q549/ | Q410K/Q549R/ | | |
| | | S586/Q594 | S586A/Q594L | | |
| | | E164/N173/ | E164G/N173T/ | | |
| | | P352/A390/ | P352T/A390V/ | | |
| | | D396/Q549/ | D396N/Q549R/ | | |
| | | S586/Q594 | S586A/Q594L | | |
| | | V83/P352/A390/ | V83G/P352T/ | | |
| | | D396/Q549/ | A390V/D396N/ | | |
| | | S586/Q594 | Q549R/ | | |
| | | | S586A/Q594L | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | T456 | T456I | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | T456/V526 | T456P/V526E | | |
| | | F43/Y349/P352/ | F43S/Y349D/ | | |
| | | A390/ | P352T/A390V/ | | |
| | | D396/H464/ | D396N/H464R/ | | |
| | | Q549/Q594/ | Q549R/Q594L/ | | |
| | | P504 | P504S | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | V526 | V526E | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | Q410/V526 | Q410K/V526E | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | Q410/V526/ | Q410K/V526E/ | | |
| | | A415/T502 | A415V/T502I | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | Q410/V526/ | Q410K/V526E/ | | |
| | | A415/T502/T21 | A415V/T502I/ | | |
| | | | T21A | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | Q410/V526/ | Q410K/V526E/ | | |
| | | A415/T502/T21/ | A415V/T502I/ | | |
| | | T273 | T21A/T273N | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | Q410/V526/A415/ | Q410K/V526E/ | | |
| | | T502/T21/R85 | A415V/T502I/ | | |
| | | | T21A/R85K | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | Q410/V526/A415/ | Q410K/V526E/ | | |
| | | T502/T21/P592 | A415V/T502I/ | | |
| | | | T21A/P592L | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | Q410/V526/A415/ | Q410K/V526E/ | | |
| | | T502/T21/R85/ | A415V/T502I/ | | |
| | | P592 | T21A/R85K/ | | |
| | | | P592L | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | Q410/V526/A415/ | Q410K/V526E/ | | |
| | | T502/I597 | A415V/T502I/ | | |
| | | | I597T | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | Q410/V526/A415/ | Q410K/V526E/ | | |
| | | T502/I597/V585 | A415V/T502I/ | | |
| | | | I597T/V585M | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | Q410/V526/A415/ | Q410K/V526E/ | | |
| | | T502/Q67 | A415V/T502I/ | | |
| | | | Q67K | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | Q410/V526/A415/ | Q410K/V526E/ | | |
| | | T502/T21/Q67 | A415V/T502I/ | | |
| | | | T21A/Q67K | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | Q410/V526/A174/ | Q410K/V526E/ | | |
| | | V208/T427/T456/ | A174S/V208M/ | | |
| | | P504 | T427S/T456I/ | | |
| | | | P504S | | |
| | | F43/Y349/P352/ | F43S/Y349D/ | | |
| | | A390/ | P352T/A390V/ | | |
| | | D396/H464/ | D396N/H464R/ | | |
| | | Q549/Q594/ | Q549R/Q594L/ | | |
| | | Q410/V526/A174/ | Q410K/V526E/ | | |
| | | V208/T427/T456/ | A174S/V208M/ | | |
| | | P504 | T427S/T456I/ | | |
| | | | P504S | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | Q410/V526/A415/ | Q410K/V526E/ | | |
| | | T502/A139 | A415V/T502I/ | | |
| | | | A139S | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | Q410/V526/A415/ | Q410K/V526E/ | | |
| | | T502/I339/F446 | A415V/T502I/ | | |
| | | | I339V/F446L | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | Q410/V526/ | Q410K/V526E/ | | |
| | | T19/D460/Q569/ | T19P/D460N/ | | |
| | | H596 | Q569R/H596L | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | Q410/V526/ | Q410K/V526E/ | | |
| | | T19/D460/Q569/ | T19P/D460N/ | | |
| | | H596/L363/T427 | Q569R/H596L/ | | |
| | | | L363M/T427A | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | Q410/V526/ | Q410K/V526E/ | | |
| | | T19/D460/Q569/ | T19P/D460N/ | | |
| | | H596/L363/E10 | Q569R/H596L/ | | |
| | | | L363M/E10K | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | Q410/V526/ | Q410K/V526E/ | | |
| | | T19/D460/Q569/ | T19P/D460N/ | | |
| | | H596/L363/E10/ | Q569R/H596L/ | | |
| | | N173 | L363M/E10K/ | | |
| | | | N173T | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | Q410/V526/ | Q410K/V526E/ | | |
| | | D460/S586/E588/ | D460N/S586F/ | | |
| | | D608 | E588K/D608N | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | Q410/V526/ | Q410K/V526E/ | | |
| | | D460/S586/E588/ | D460N/S586F/ | | |
| | | D608/H596 | E588K/D608N/ | | |
| | | | H596L | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | Q410/V526/ | Q410K/V526E/ | | |
| | | D460/S586/E588/ | D460N/S586F/ | | |
| | | D608/H596/L26 | E588K/D608N/ | | |
| | | | H596L/L26M | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | Q410/V526/ | Q410K/V526E/ | | |
| | | D460 | D460N | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | A174/T427 | A174S/T427S | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | V208 | V208M | | |
| | | F43/Y349/P352/ | F43S/Y349N/ | | |
| | | A390/ | P352T/A390V/ | | |
| | | D396/H464/ | D396N/H464R/ | | |
| | | Q549/Q594/ | Q549R/Q594L/ | | |
| | | R63/A145/I182/ | R63G/A145S/ | | |
| | | V526 | I182T/V526E | | |
| | | P352/A390/ | P352T/A390V/ | | |
| | | D396 | D396N | | |
| | | A283/Y349/ | A283T/Y349H/ | | |
| | | D396/Q594 | D396N/Q594L | | |
| | | F8/F43/A174/ | F8S/F43S/A174S/ | | |
| | | Y349/P352/ | Y349N/P352T/ | | |
| | | A390/D396/ | A390V/D396N/ | | |
| | | T427/H464/ | T427S/H464R/ | | |
| | | Q549/Q594 | Q549R/Q594L | | |

TABLE 3

| | | Amino | | | | | |
|---|---|---|---|---|---|---|---|
| Amino | | Acid of | | Amino | | Amino | |
| Acid of | | WT Cas8- | | Acid of | | Acid of | |
| WT TniQ | | Cas5 fusion | | WT Cas7 | | WT Cas6 | |
| (SEQ ID | Amino Acid | (SEQ ID | Amino Acid | (SEQ ID | Amino Acid | (SEQ ID | Amino Acid |
| NO: 7) | Modification | NO: 8) | Modification | NO: 9) | Modification | NO: 10) | Modification |
| M99 | M99I | Y119 | Y119H | R28 | R28K | A21 | A21S |
| R133 | R133* | N134 | N134Q | A82 | A82T | V90 | V90A |
| S189 | S189N | N134 | N134R | K144 | K144E | | |
| H265 | H265Q | D155 | D155N | C151 | C151R | | |
| A266 | A266V | Q180 | Q180R | N162 | N162S | | |
| L336 | L336F | D183 | D183N | K182 | K182E | | |
| V343 | V343A | R274 | R274L | D273 | D273G | | |
| | | N319 | N319D | A327 | A327D | | |
| | | V447 | V447I | M346 | M346I | | |
| | | A454 | A454S | | | | |
| | | E458 | E458G | | | | |
| | | D461 | D461N | | | | |
| | | A512 | A512T | | | | |
| | | D538 | D538K | | | | |
| | | P580 | P580Q | | | | |
| | | D155/Q180 | D155N/Q180R | | | | |

TABLE 4

| | | Amino | | | | | |
|---|---|---|---|---|---|---|---|
| Amino | | Acid of | | Amino | | Amino | |
| Acid of | | WT Cas8- | | Acid of | | Acid of | |
| WT TniQ | | Cas5 fusion | | WT Cas7 | | WT Cas6 | |
| (SEQ ID | Amino Acid | (SEQ ID | Amino Acid | (SEQ ID | Amino Acid | (SEQ ID | Amino Acid |
| NO: 11) | Modification | NO: 12) | Modification | NO: 13) | Modification | NO: 14) | Modification |
| A2 | A2T | K4 | K4N | N5 | N5K | Q2 | Q2K |
| F3 | F3S | K4/R49 | K4N/R49L | N5 | N5T | H9 | H9L |
| P7 | P7R | K4/M388 | K4N/M388V | D10 | D10N | K13 | K13E |
| A9 | A9S | K4/E571 | K4N/E571D | R11 | R11K | Q14 | Q14K |
| A9 | A9G | K4/A162/ | K4N/A162T/ | D26 | D26N | A15 | A15G |
| | | G480 | G480D | | | | |
| A11 | A11G | K4/Q315 | K4N/Q315R | V30 | V30E | K34 | K34N |
| F12 | F12I | E5 | E5K | V30/C121 | V30E/C121F | E38 | E38K |
| D14 | D14N | E5/R316 | E5K/R316G | V30/F46/ | V30E/F46V/ | V42 | V42I |
| | | | | A240/ | A240T/ | | |
| | | | | K304/ | K304R/ | | |
| | | | | C316 | C316G | | |
| S16 | S16Y | L6 | L6M | V30/F46/ | V30E/F46V | A46 | A46D |
| | | | | A240/ | A240T/ | | |
| | | | | C316 | C316G | | |
| Y20 | Y20H | L6 | L6I | D35 | D35N | S50 | S50I |
| S26 | S26N | E8 | E8K | R40 | R40L | V59 | V59G |
| F29 | F29S | E8 | E8D | P42 | P42A | Y60 | Y60H |
| S32 | S32N | I9 | I9T | P42/T318 | P42A/T318P | A73 | A73S |
| E34 | E34K | D11 | D11N | G45 | G45S | A73 | A73T |
| G35 | G35V | T12 | T12A | G45 | G45V | F75 | F75L |
| G35 | G35S | T13 | T13I | F46 | F46V | D77 | D77G |
| G35 | G35D | D16 | D16G | T47 | T47R | G82 | G82S |
| I40 | I40S | R17 | R17C | T47 | T47S | G82/I110/ | G82S/I110S/ |
| | | | | | | S115/ | S115R/ |
| | | | | | | H164/ | H164Y/ |
| | | | | | | S199 | S199I |
| E43 | E43D | R17 | R17S | N58 | N58T | G82/I110/ | G82S/I110S/ |
| | | | | | | S115/ | S115R/ |
| | | | | | | K124/ | K124R/ |
| | | | | | | H164/ | H164Y/ |
| | | | | | | S199 | S199I |
| H45 | H45P | R17/S156 | R17S/S156G | P61 | P61L | F83 | F83L |
| E46 | E46K | R20 | R20K | T65 | T65I | F83 | F83V |
| A54 | A54S | R20 | R20E | T71 | T71I | F83 | F83C |
| R61 | R61W | R21 | R21E | T71 | T71R | K85 | K85E |
| V64 | V64M | R21 | R21K | T71 | T71D | V86 | V86I |
| Y65 | Y65C | S24 | S24K | L72 | L72M | E97 | E97 |
| N70 | N70S | S24 | S24Q | C75 | C75S | I110 | I110S |
| A77 | A77T | S24 | S24R | V77 | V77A | I110 | I110L |
| D101 | D101N | Y26 | Y26S | P78 | P78L | I110/ | I110S/ |
| | | | | | | S115/ | S115R/ |
| | | | | | | H164/ | H164F/ |
| K103 | K103E | Y26 | Y26H | N80 | N80T | I110/ | I110S/ |
| | | | | | | S115/ | S115R/ |
| | | | | | | H164/ | H164F/ |
| | | | | | | S199 | S199I |
| N105 | N105K | A28 | A28S | E82 | E82D | I110/ | I110S/ |
| | | | | | | S115/ | S115R/ |
| | | | | | | H164/ | H164F/ |
| | | | | | | S199/ | S199I/ |
| | | | | | | K124 | K124R |
| N105 | N105D | A28 | A28D | H83 | H83Y | S115 | S115R |
| N105/ | N105K/ | M29 | M29I | H83 | H83N | K120 | K120N |
| A109/ | A109G/ | | | | | | |
| D131/ | D131N/ | | | | | | |
| Q148/ | Q148R/ | | | | | | |
| M279/ | M279I/ | | | | | | |
| S310 | S310P | | | | | | |
| S106 | S106G | G34 | G34D | A94 | A94S | K124 | K124R |
| V108 | V108M | A37 | A37S | V98 | V98M | G130 | G130D |
| A109 | A109G | V38 | V38M | E113 | E113D | D132 | D132E |
| Y111 | Y111N | V38 | V38G | A115 | A155S | N134 | N134T |
| L119 | L119M | V38/S108/ | V38G/S108P/ | E116 | E116D | A140 | A140T |
| | | A497/S583 | A497S/S583R | | | | |
| R120 | R120S | I41 | I41V | T117 | T117I | E143 | E143K |
| R123 | R123S | R49 | R49L | C121 | C121F | D145 | D145G |
| A126 | A126T | D54 | D54G | A128 | A128S | S156 | S156I |
| E127 | E127G | K59 | K59R | R133 | R133K | E159 | E159K |
| V130 | V130M | K59/D157/ | K59R/D157N/ | G138 | G138V | I162 | I162V |
| | | S644 | S644N | | | | |
| D131 | D131N | K60 | K60N | N146 | N146D | H164 | H164Y |
| Q148 | Q148R | K63 | K63N | G148 | G148V | H164 | H164F |
| S149 | S149Y | A65 | A65T | C161 | C161R | Y177 | Y177C |
| H151 | H151Y | A65 | A65V | A171 | A171V | S199 | S199I |
| A157 | A157D | K67 | K67E | A171 | A171S | S232 | S232L |
| T159 | T159I | K67/K96/ | K67E/K96N/ | K175 | K175T | L270 | L270S |
| | | V170/G303/ | V170E/G303D/ | | | | |
| | | Q315/N494/ | Q315R/N494D/ | | | | |
| | | I672 | I672V | | | | |
| A164 | A164V | K74 | K74E | A177 | A177V | | |
| L166 | L166M | K77 | K77E | K182 | K182E | | |
| T185 | T185A | W81 | W81C | L184 | L184M | | |
| S194 | S194G | K88 | K88R | L184/ | L184M/ | | |
| | | | | A240/ | A240V/ | | |
| | | | | N315/ | N315K/ | | |
| | | | | A345 | A345T | | |
| A196 | A196T | K88 | K88E | I191 | I191V | | |
| T203 | T203A | I92 | I92T | S193 | S193A | | |
| K211 | K211R | R93 | R93E | S193 | S193F | | |
| E217 | E217K | R93 | R93K | F201 | F201S | | |
| R218 | R218K | V94 | V94M | S203 | S203N | | |
| R218 | R218S | K96 | K96N | E211 | E211K | | |
| N219 | N219S | K96/I305/ | K96N/I305T/ | E211/R274 | E211K/R274G | | |
| | | K550/V642 | K550N/V642D | | | | |
| A236 | A236T | K96/V170/ | K96N/V170E/ | A212 | A212V | | |
| | | G303/Q315/ | G303D/Q315R/ | | | | |
| | | N494/I672 | N494D/I6721 | | | | |
| E242 | E242D | K96/K171/ | K96N/K171E/ | Y219 | Y219R | | |
| | | V289/Q315 | V289M/Q315R | | | | |
| N257 | N257K | K96/K171/ | K96N/K171E/ | N225 | N225T | | |
| | | V289/G303/ | V289M/G303D/ | | | | |
| | | Q315 | Q315R | | | | |
| N267 | N267S | K96/K160/ | K96N/K160E/ | N225 | N225S | | |
| | | K181/R276/ | K181T/R276G/ | | | | |
| | | G673 | G673V | | | | |
| M279 | M279I | E102 | E102D | D226 | D226Y | | |
| M279 | M279V | E102 | E102G | E232 | E232K | | |
| D283 | D283G | T105 | T105A | E232 | E232Q | | |
| N286 | N286S | L106 | L106M | A233 | A233N | | |
| T288 | T288I | L106/K160/ | L106M/K160E/ | A233 | A233S | | |
| | | I128 | I228V | | | | |
| K291 | K291Q | S108 | S108P | A233 | A233K | | |
| I293 | I293V | V110 | V110A | K235 | K235R | | |
| D296 | D296N | G121 | G121S | K235/T318 | K235R/T318P | | |
| S303 | S303I | S126 | S126P | Q236 | Q236R | | |
| S303 | S303G | K128 | K128R | Q236 | Q236S | | |
| K306 | K306N | L134 | L134M | F237 | F237L | | |
| S310 | S310Y | L134/ | L134M/ | F237/V238 | F237L/V238M | | |
| | | T179/ | T179A/ | | | | |
| | | P185/ | P185T/ | | | | |
| | | Y540/ | Y540C/ | | | | |
| | | K555/ | K555E/ | | | | |
| | | K624/ | K624N/ | | | | |
| | | E646 | E646D | | | | |
| S310 | S310P | L134/ | L134M/ | V238 | V238Q | | |
| | | T179/ | T179A/ | | | | |
| | | P185/ | P185T/ | | | | |
| | | Y540/ | Y540C/ | | | | |
| | | K555/ | K555E/ | | | | |
| | | E646 | E646D | | | | |
| I313 | I313T | Y138 | Y138S | V238 | V238M | | |
| Y314 | Y314F | Y138/ | Y138S/ | A240 | A240T | | |
| | | A250/ | A250S/ | | | | |
| | | S275/ | S275N/ | | | | |
| | | D421 | D421N | | | | |
| A316 | A316T | Q142 | Q142H | A240 | A240V | | |
| E326 | E326G | W147 | W147L | S250 | S250A | | |
| T331 | T331I | K150 | K150N | R274 | R274G | | |
| A336 | A336V | V151 | V151M | A282 | A282V | | |
| A347 | A347T | V151 | V151L | I286 | I286N | | |
| A347 | A347S | A153 | A153T | I286 | I286T | | |
| T352 | T352S | S156 | S156R | I286 | I286F | | |
| Y361 | Y361H | S156 | S156G | I286/N315 | I286F/N315S | | |
| M374 | M374T | D157 | D157N | P292 | P292S | | |
| M374 | M374I | K160 | K160R | S295 | S295N | | |
| R377 | R377G | K160 | K160E | K304 | K304R | | |
| T395 | T395I | K160/R198/ | K160E/R198S/ | E307 | E307D | | |
| | | G303/Q315 | G303D/Q315R | | | | |
| S396 | S396T | K160/K181/ | K160E/K181T/ | Y309 | Y309C | | |
| | | G673 | G673V | | | | |
| S396 | S396F | K160/K181/ | K160E/K181T/ | A312 | A312V | | |
| | | N323/G673 | N323S/G673V | | | | |
| G398 | G398V | A162 | A162T | L313 | L313M | | |
| A408 | A408V | S165 | S165N | N315 | N315T | | |
| 410 | 410L | S165 | S165G | N315 | N315K | | |
| A9/N105/ | A9S/N105K/ | V170 | V170E | N315 | N315S | | |
| A109/ | A109G/ | | | | | | |
| D131/ | D131N/ | | | | | | |
| Q148/ | Q148R/ | | | | | | |
| M279/ | M279I/ | | | | | | |
| S310 | S310P | | | | | | |
| | | K171 | K171E | C316 | C316G | | |
| | | F173 | F173V | I317 | I317V | | |
| | | K174 | K174N | I317/A347 | I317V/A347D | | |
| | | K174 | K174R | T318 | T318A | | |
| | | T179 | T179A | T318 | T318P | | |
| | | K181 | K181T | K320 | K320R | | |
| | | S183 | S183N | N321 | N321D | | |
| | | P185 | P185T | E322 | E322K | | |
| | | E186 | E186K | K323 | K323N | | |
| | | E186 | E186D | I328 | I328T | | |
| | | E187 | E187K | I328/A350 | I328T/A350V | | |
| | | A188 | A188S | M340 | M340I | | |
| | | A188 | A188V | K343 | K343E | | |
| | | D191 | D191Y | K343 | K343R | | |
| | | D191 | D191E | K344 | K344E | | |
| | | R198 | R198H | K344 | K344R | | |
| | | R198 | R198C | A345 | A345T | | |
| | | R198 | R198S | A345 | A345D | | |
| | | R201 | R201K | A345 | A345Y | | |
| | | D206 | D206G | A345 | A345S | | |
| | | G207 | G207D | A345 | A345R | | |
| | | A226 | A226T | A345 | A345K | | |
| | | I228 | I228V | A345 | A345E | | |
| | | R233 | R233K | A345 | A345G | | |
| | | N236 | N236T | A347 | A347S | | |
| | | R241 | R241E | A347 | A347K | | |
| | | A249 | A249S | A347 | A347D | | |
| | | A250 | A250S | K348 | K348N | | |
| | | I256 | I256T | K349 | K349R | | |
| | | S267 | S267G | A350 | A350K | | |
| | | S267 | S267N | A350 | A350V | | |
| | | K268 | K268N | A350 | A350D | | |
| | | H270 | H270P | A350 | A350T | | |
| | | S275 | S275N | I286/ | I286N/ | | |
| | | | | A350 | A350D | | |
| | | S275 | S275G | A171/I286/ | A171S/I286F/ | | |
| | | | | N315 | N315S | | |
| | | R276 | R276G | | | | |
| | | A277 | A277D | | | | |
| | | A277 | A277S | | | | |
| | | A277 | A277T | | | | |
| | | K279 | K279N | | | | |
| | | G283 | G283D | | | | |
| | | V286 | V286G | | | | |
| | | V289 | V289M | | | | |
| | | G303 | G303D | | | | |
| | | I305 | I305T | | | | |
| | | F306 | F306S | | | | |
| | | A310 | A310D | | | | |
| | | A310 | A310T | | | | |
| | | A312 | A312G | | | | |
| | | A312 | A312D | | | | |
| | | A312 | A312T | | | | |
| | | A312/H4242/ | A312T/H424N/ | | | | |
| | | A449/G457 | A449T/G457D | | | | |
| | | K314 | K314N | | | | |
| | | Q315 | Q315R | | | | |
| | | R316 | R316G | | | | |
| | | N323 | N323S | | | | |
| | | E326 | E326A | | | | |
| | | E326 | E326K | | | | |
| | | N329 | N329S | | | | |
| | | G349 | G349D | | | | |
| | | E353 | E353D | | | | |
| | | L355 | L355M | | | | |
| | | L355 | L355R | | | | |
| | | E356 | E356G | | | | |
| | | E356 | E356D | | | | |
| | | S357 | S357P | | | | |
| | | A358 | A358V | | | | |
| | | R361 | R361S | | | | |
| | | P370 | P370T | | | | |
| | | N372 | N372K | | | | |
| | | E373 | E373D | | | | |
| | | S376 | S376F | | | | |
| | | S376/ | S376F/ | | | | |
| | | D611 | D611N | | | | |
| | | T378 | T378I | | | | |
| | | F382 | F382L | | | | |
| | | M388 | M388V | | | | |
| | | G391 | G391S | | | | |
| | | R397 | R397K | | | | |
| | | A399 | A399S | | | | |
| | | K403 | K403N | | | | |
| | | M405 | M405I | | | | |
| | | L419 | L419P | | | | |
| | | D421 | D421N | | | | |
| | | K423 | K423R | | | | |
| | | H424 | H424N | | | | |
| | | H424 | H424R | | | | |
| | | V425 | V425L | | | | |
| | | I427 | I427V | | | | |
| | | E428 | E428K | | | | |
| | | D430 | D430A | | | | |
| | | D431 | D431G | | | | |
| | | E432 | E432D | | | | |
| | | H433 | H433N | | | | |
| | | A449 | A449T | | | | |
| | | G457 | G457D | | | | |
| | | R473 | R473K | | | | |
| | | E477 | E477D | | | | |
| | | G480 | G480D | | | | |
| | | F485 | F485L | | | | |
| | | S487 | S487R | | | | |
| | | S487 | S487G | | | | |
| | | S489 | S489N | | | | |
| | | N494 | N494D | | | | |
| | | S496 | S496N | | | | |
| | | A497 | A497S | | | | |
| | | V498 | V498G | | | | |
| | | K500 | K500N | | | | |
| | | K502 | K502N | | | | |
| | | Q509 | Q509R | | | | |
| | | A511 | A511T | | | | |
| | | A511 | A511E | | | | |
| | | R515 | R515S | | | | |
| | | R518 | R518S | | | | |
| | | P519 | P519T | | | | |
| | | G520 | G520D | | | | |
| | | G520 | G520V | | | | |
| | | Y540 | Y540C | | | | |
| | | Q545 | Q545H | | | | |
| | | K550 | K550N | | | | |
| | | K555 | K555E | | | | |
| | | H557 | H557Q | | | | |
| | | P570 | P570S | | | | |
| | | E571 | E571D | | | | |
| | | C580 | C580R | | | | |
| | | S583 | S583R | | | | |
| | | E585 | E585K | | | | |
| | | E585 | E585G | | | | |
| | | E590 | E590D | | | | |
| | | R594 | R594K | | | | |
| | | M603 | M603I | | | | |
| | | H607 | H607N | | | | |
| | | H607 | H607L | | | | |
| | | K608 | K608R | | | | |
| | | D611 | D611N | | | | |
| | | L617 | L617P | | | | |
| | | N620 | N620S | | | | |
| | | K624 | K624N | | | | |
| | | T636 | T636P | | | | |
| | | M639 | M639V | | | | |
| | | N641 | N641S | | | | |
| | | V642 | V642G | | | | |
| | | S644 | S644N | | | | |
| | | S644 | S644G | | | | |
| | | E646 | E646D | | | | |
| | | A655 | A655V | | | | |
| | | V658 | V658M | | | | |
| | | K660 | K660N | | | | |
| | | T663 | T663A | | | | |
| | | T665 | T665I | | | | |
| | | R668 | R668S | | | | |
| | | I672 | I672V | | | | |
| | | G673 | G673V | | | | |
| | | S678 | S678R | | | | |
| | | M682 | M682L | | | | |
| | | A685 | A685V | | | | |
| | | A685 | A685D | | | | |
| | | K688 | K688N | | | | |
| | | V695 | V695M | | | | |
| | | G303/ | G303D/ | | | | |
| | | M405/ | M405I/ | | | | |
| | | G520/ | G520D/ | | | | |
| | | E590 | E590D | | | | |

Tn6677-TnsA
(SEQ ID NO: 1)
MATSLPTPSAITTSALEYAFHTPARNLTKSRGKNIHRYVSVKMSKRITVESTLECDACYH
FDFEPSIVRFCAQPIRFLYYLNGQSHSYVPDFLVQFDTNEFVLYEVKSAYAKNKPDFDVE
WEAKVKAATELGLELELVEESDIRDTVVLNNLKRMHRYASKDELNNVHNSLLKIIKYN
GAQSARCLGEQLGLKGRTVLPILCDLLSRCLLDTRLDKPLSLESRFELASYG
Tn6677-TnsB
(SEQ ID NO: 2)
MAKKGFSSFHRKAVSSQDTLESIELVSSANCLESVTYQDISAFPETIAVEINFRLSILRFLA
RKCETIVAKSIEPHRVELQQNYSRKIPSAITIYRWWLAFRKSDYNPISLAPNIKDRGNRET
KVSTVVDSIMEQAVERVISGRKVNVSSAYKRVRRKVRQYNLTHGTKYTYPKYESVRKR
VKKKTPFELLAAGKGERVAKREFRRMGKKILTSSVLERVEIDHTVVDLFAVHEEYRIPL
GRPWLTQLVDCYSKAVIGFYLGFEPPSYVSVSLALKNAIQRKDDLISSYESIENEWLCYG
IPDLLVTDNGKEFLSKAFDQACESLLINVHQNKVETPDNKPHVERNYGTINTSLLDDLPG
KSFSQYLQREGYDSVGEATLTLNEIREIYLIWLVDIYHKKPNQRGTNCPNVAWKKGCQE
WEPEEFSGSKDELDFKFAIVDYKQLTKVGITVYKELSYSNDRLAEYRGKKGNHKVQFK
YNPECMAVIWVLDEDMNEYFTVNAIDYEYASRVSLWQHKYNMKYQAELNSAEYDED
KEIDAEIKIEEIADRSIVKTNKIRARRRGARHQENSARAKSISNANPASIQKHEDEIVSADN
DDWDIDYV
Tn6677-TnsC
(SEQ ID NO: 3)
MSETREARISRAKRAFVSTPSVRKILSYMDRCRDLSDLESEPTCMMVYGASGVGKTTVI
KKYLNQNRRESEAGGDIIPVLHIELPDNAKPVDAARELLVEMGDPLALYETDLARLTKR
LTELIPAVGVKLIIIDEFQHLVEERSNRVLTQVGNWLKMILNKTKCPIVIFGMPYSKVVLQ
ANSQLHGRFSIQVELRPFSYQGGRGVFKTFLEYLDKALPFEKQAGLANESLQKKLYAFS
QGNMRSLRNLIYQASIEAIDNQHETITEEDFVFASKLTSGDKPNSWKNPFEEGVEVTEDM
LRPPPKDIGWEDYLRHSTPRVSKPGRNKNFFE
Tn7016-TnsA
(SEQ ID NO: 4)
MYIRNLRKPSPNKNVFKFASTKVSSVVMCESSLEFDACFHHEYNDLIESFGSQPEGFKYE
FMGKSLPYTPDALISYTDKTQKYHEYKPYSKIASPLFRAEFAAKRAASLKLGIDLVLVTD
RQIRVNPILNNLKLLHRYSGVYGISGIQKELLSFIHKSGVIKLNDISSQVGIPIGETRSFLFG
LMHKGLVKADLGCDDLTNNPTLWATP
Tn7016-TnsB
(SEQ ID NO: 5)
MTDFFNEFDESLVPLKPQTPTQYVKLDDANLIQRDLDTFSDTFKNQALQRYKLISTIDKK
LSRGWTQRNLDPILDELFKGGDVVRPNWRTVARWRKKYIESNGDIASLADKNHKMGN
RTNRIKGDDKFFDKALERFLDAKRPTIATAYQYYKDLIVIENESIVEGKIPIISYNAFNKRI
KAIPPYAVAVARHGKFKADQWFAYCAAHVPPTRILERVEIDHTPLDLILLDDELLIPIGRP
YLTLLIDVFSGCVLGFHLSYKSPSYVSAAKAITHAIKPKSLDALNIELQNDWPCFGKFEN
LVVDNGAEFWSKNLEHACQSAGINIQYNPVRKPWLKPFIERFFGVMNEYFLPELPGKTF
SNILEKEEYKPEKDAIMRFSTFVEEFHRWIADVYHQDSNSRETRIPIKRWQQGFDAYPPL
TMNEEEETRESMLMRISDSRTLTRNGFKYQELMYDSTALADYRKHYPQTKETVKKLIK
VDPDDISKIYVYLEELESYLEVPCTDPTGYTDGLSIYEHKTIKKINREVIRESKDSLGLAK
ARMAIHERVKQEQEVFIESKTKAKITAVKKQAQIADVSNTGTSTIKVSEESAAPVQKHIS
NDNSDDWDDDLEAFE
Tn7016-TnsC
(SEQ ID NO: 6)
MNALTEIQIEKLRNFSDCIVMHPQIKTIFNDFDELRLNRKFQSDQQCMLLIGDTGVGKSH
TINHYKKRVLATQNYSRNTMPVLVSRISRGKGLDATLVQMLADLELFGSSQIKKRGYKT
DLTKKLVESLIKAQVELLIINEFQELIEFKSVQERQQIANGLKFISEEAKVPIVLVGMPWA
AKIAEEPQWASRLVRKRKLEYFSLKNDSKYFRQYLMGLAKKMPFDVPPKLESKNTTIAL
FAACRGENRALKHLLLEALKLALSCNEYLENKHFITAYDKFDFFNDKEKLKSKNPFKQD
IKDIEIYEVIKNSSYNPNALDPEDMLTDRVFAIVK
Tn6677-TniQ
(SEQ ID NO: 7)
MFLQRPKPYSDESLESFFIRVANKNGYGDVHRFLEATKRFLQDIDHNGYQTFPTDITRIN
PYSAKNSSSARTASFLKLAQLTFNEPPELLGLAINRTNMKYSPSTSAVVRGAEVFPRSLL
RTHSIPCCPLCLRENGYASYLWHFQGYEYCHSHNVPLITTCSCGKEFDYRVSGLKGICCK
CKEPITLTSRENGHEAACTVSNWLAGHESKPLPNLPKSYRWGLVHWWMGIKDSEFDHF
SFVQFFSNWPRSFHSIIEDEVEFNLEHAVVSTSELRLKDLLGRLFFGSIRLPERNLQHNIIL
GELLCYLENRLWQDKGLIANLKMNALEATVMLNCSLDQIASMVEQRILKPNRKSKPNS
PLDVTDYLFHFGDIFCLWLAEFQSDEFNRSFYVSRW
Tn6677-Cas8-Cas5 fusion
(SEQ ID NO: 8)
MQTLKELIASNPDDLTTELKRAFRPLTPHIAIDGNELDALTILVNLTDKTDDQKDLLDRA
KCKQKLRDEKWWASCINCVNYRQSHNPKFPDIRSEGVIRTQALGELPSFLLSSSKIPPYH
WSYSHDSKYVNKSAFLTNEFCWDGEISCLGELLKDADHPLWNTLKKLGCSQKTCKAM
AKQLADITLTTINVTLAPNYLTQISLPDSDTSYISLSPVASLSMQSHFHQRLQDENRHSAIT
RFSRTTNMGVTAMTCGGAFRMLKSGAKFSSPPHHRLNSKRSWLTSEHVQSLKQYQRLN
KSLIPENSRIALRRKYKIELQNMVRSWFAMQDHTLDSNILIQHLNHDLSYLGATKRFAYD
PAMTKLFTELLKRELSNSINNGEQHTNGSFLVLPNIRVCGATALSSPVTVGIPSLTAFFGF
VHAFERNINRTTSSFRVESFAICVHQLHVEKRGLTAEFVEKGDGTISAPATRDDWQCDV
VFSLILNTNFAQHIDQDTLVTSLPKRLARGSAKIAIDDFKHINSFSTLETAIESLPIEAGRW
LSLYAQSNNNLSDLLAAMTEDHQLMASCVGYHLLEEPKDKPNSLRGYKHAIAECIIGLI
NSITFSSETDPNTIFWSLKNYQNYLVVQPRSINDETTDKSSL
Tn6677-Cas7
(SEQ ID NO: 9)
MKLPTNLAYERSIDPSDVCFFVVWPDDRKTPLTYNSRTLLGQMEAASLAYDVSGQPIKS
ATAEALAQGNPHQVDFCHVPYGASHIECSFSVSESSELRQPYKCNSSKVKQTLVQLVEL
YETKIGWTELATRYLMNICNGKWLWKNTRKAYCWNIVLTPWPWNGEKVGFEDIRTNY
TSRQDFKNNKNWSAIVEMIKTAFSSTDGLAIFEVRATLHLPTNAMVRPSQVFTEKESGSK
SKSKTQNSRVFQSTTIDGERSPILGAFKTGAAIATIDDWYPEATEPLRVGRFGVHREDVT
CYRHPSTGKDFFSILQQAEHYIEVLSANKTPAQETINDMHFLMANLIKGGMFQHKGD
Tn6677-Cas6
(SEQ ID NO: 10)
VKWYYKTITFLPELCNNESLAAKCLRVLHGFNYQYETRNIGVSFPLWCDATVGKKISFV
SKNKIELDLLLKQHYFVQMEQLQYFHISNTVLVPEDCTYVSFRRCQSIDKLTAAGLARKI
RRLEKRALSRGEQFDPSSFAQKEHTAIAHYHSLGESSKQTNRNFRLNIRMLSEQPREGNS
IFSSYGLSNSENSFQPVPLI
Tn7016-TniQ
(SEQ ID NO: 11)
MAFLFSPKARAFSDESLESYLLRVVSENFFDSYEGLSLAIREELHELDFEAHGAFPVDLK
RLNVYHAKHNSHFRMRALGLLETLLDLPRYELQKLALLKSDIKENSSVALYNNGVDIPL
RFIRHHAEEAVDSIPVCSQCLAEEAYIKQSWHIKWVNACTKHQCALLHNCPECYAPINYI
ENESITHCSCGFELSCASTSPVNTLSIEHLNKLLDKGERNDSNPLFNNMTLTERFAALLW
YQERYSQTDNFCLNDAVNYFSKWPAVENTELDELSKNAEMKLIDLENKTEFKFIFGDAI
LACPSTQKQSESHFIYRALLDYLVTLVESNPKTKKPNAADLLVSVLEAATLLGTSVEQV
YRLYQNGILQTAFRHKMNQRINPYKGAFFLRHVIEYKTSFGNDKARMYLSAW
Tn7016-Cas8-Cas5 fusion
(SEQ ID NO: 12)
MHLKELLEITDTTERDRSLRRAFSPYTAMIDITGSEAVALIILLNLTYRKNQVDDLLDKKL
AKQALKSEDHINKCIKEIAWFHTHNLKYPDIRVSKQNLAVEPPTLHSYVLSSANYPKAY
GWSHNSAKVNFAKLFVSYFKWQNQVSWLAQVLATNSDNWKSAFTSLGLSVKAFKSLC
VTVKNSLPEEAIPDSVDRYSRQIRMPYHDGYLAVTPVISHVVQSKIQQAAIDKRARFSNV
EFTRPAAVSMLAASLGGVINVLNYPPYIRSKYHGLSNSRAFKLNNGQTVENVEALLKPE
LIKALEGIIFSNNALALKQRRQQKVKNIKELRNTLLEWFSPVFEWRLDAIENGYDLEQLE
SASERLEYKILSLPDNELPSLTIPLFRLLNEMLGGVSMTQRYAFHPKLMSPLKAALQWLL
VNLTDQKHVLIEEDDEHYRYLHLSGIRVFDAQALSNPYCSGIPSLTAVWGMIHSYQRKL
NEALGTNVRFTSFSWFIRNYSAVAGKKLPELSLQGAQQSRLKRPGIIDGKYCDLVEDLII
HIDGYEDDLQAVDSKPDILKAHFPSNFAGGVMHQPELNSNINWCCLYSNENQLFEKLRR
LPLSGCWVMPTEHKIQDLDELLLLLNSDSKLSPSMMGYMLLTEPMARVGSLERLHCYA
EPAIGVVKYEAATSVRLKGIGNYFNSAFWMLDAQEKFMLMKKV
Tn7016-Cas7
(SEQ ID NO: 13)
MELCNILKYDRSLYPGKAVFFYKTADSDFVPLEADINKIRGPKSGFTEAFTPQFSPKNISP
QDLTHNNILTLEECYVPPNVEHIFCRFSLRVQANSLVPSGCSDPEVFSLLKELAETFKECG
GYKELAVRYCRNILIGTWLWRNQNTGNTQIEIKTSKGSCYLIDNTRKLAWESKWASDDL
KVLEELSNEIESALTDPNVFWSADITAKIEASFCQEIYPSQILNDKVKQGEASKQFVKAKC
ADGRYAVSFNSVKIGAALQSIDDWWDEDASKRLRVHEFGADKEIGVARRPPDSEQNFY
SIFKNTEWYLSALKNCITNKNEKIDPAIYYLFSVLIKGGMFQKKAEAKKA
Tn7016-Cas6
(SEQ ID NO: 14)
MQRYYFTVHFLPKQANLALLTGRCISIMHGFILKHNIEGMGVTFPAWSDSSIGNEIAFVY
TDKEILNTLKDQAYFVDMQDCGFFKVSQVLAVPDSCEEVRFIRNQAVAKIFTGESRRRL
KRLQKRALARGEDFNPKKIEAPREIDIFHRVAMTSKSSQEDYILHIQKQDVDCQAEPYFS
NYGLASNEKFKGTVPDLSPSIDRN

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

1. A polypeptide comprising one or more amino acid sequences having at least 70% identity to any of SEQ ID NOs: 1-14 with one or more amino acid substitutions, deletions, or additions relative to SEQ ID NOs: 1-14.

2. A polypeptide of claim 1, comprising an amino acid sequence having:
a) at least 70% identity to SEQ ID NO: 1 and one or more amino acid substitutions at positions: 2, 3, 5, 28, 57, 77, 80, 107, 110, 116, 122, 142, 155, 161, 166, 173, 177, 185, 211, 216, 227, and 230, relative to SEQ ID NO: 1;
b) at least 70% identity to SEQ ID NO: 2 and one or more amino acid substitutions at positions: 2, 5, 22, 24, 25, 29, 75, 141, 199, 215, 319, 347, 364, 370, 383, 439, 454, 458, 485, 509, 533, 538, 565, 581, 586, 595, 596, 597, and 600, relative to SEQ ID NO: 2;
c) at least 70% identity to SEQ ID NO: 3 and one or more amino acid substitutions at positions: 9, 15, 16, 18, 21, 64, 81, 86, 87, 99, 109, 142, 147, 153, 168, 180, 216, 230, 285, and 304, relative to SEQ ID NO: 3;
d) at least 70% identity to SEQ ID NO: 4 and one or more amino acid substitutions at positions: 4, 5, 9, 10, 12, 21, 23, 25, 26, 31, 32, 34, 35, 37, 41, 45, 47, 48, 51, 52, 55, 60, 61, 65, 67, 69, 72, 75, 79, 80, 82, 87, 88, 90, 91, 93, 94, 96, 98, 99, 100, 103, 106, 108, 113, 116, 125, 126, 128, 129, 135, 139, 143, 146, 147, 149, 153, 154, 156, 158, 159, 160, 162, 164, 166, 167, 168, 169, 170, 177, 179, 180, 182, 183, 185, 187, 188, 190, 191, 192, 193, 195, 196, 200, 204, 207, and 208, relative to SEQ ID NO: 4;
e) at least 70% identity to SEQ ID NO: 5 and one or more amino acid substitutions at positions: 1, 2, 4, 5, 8, 9, 10, 11, 12, 13, 14, 15, 16, 17, 19, 20, 21, 22, 23, 24, 25, 26, 27, 28, 29, 30, 32, 33, 36, 37, 39, 40, 41, 42, 43, 44, 45, 49, 52, 55, 56, 58, 60, 62, 63, 67, 71, 74, 76, 78, 79, 80, 81, 82, 83, 84, 85, 86, 87, 88, 89, 91, 92, 95, 97, 100, 101, 104, 106, 110, 112, 113, 115, 117, 119, 120, 124, 125, 127, 129, 130, 131, 134, 139, 142, 144, 145, 146, 147, 149, 150, 155, 156, 157, 158, 159, 163, 164, 165, 167, 169, 173, 174, 176, 181, 182, 186, 187, 190, 195, 197, 198, 205, 208, 209, 211, 215, 218, 223, 226, 227, 231, 232, 235, 239, 246, 248, 250, 259, 260, 261, 262, 263, 267, 269, 273, 274, 277, 278, 280, 281, 282, 283, 285, 287, 288, 290, 295, 298, 302, 303, 307, 313, 316, 317, 320, 323, 325, 331, 332, 339, 345, 348, 349, 352, 353, 354, 356, 361, 362, 363, 364, 365, 366, 367, 369, 370, 371, 372, 373, 375, 376, 380, 383, 385, 386, 389, 390, 392, 396, 397, 399, 402, 403, 404, 407, 408, 410, 411, 412, 413, 414, 415, 416, 421, 422, 423, 424, 425, 426, 427, 428, 429, 430, 431, 434, 435, 437, 440, 443, 445, 446, 448, 450, 452, 456, 459, 460, 463, 464, 470, 472, 473, 494, 495, 498, 501, 502, 504, 505, 506, 508, 509, 510, 512, 513, 514, 517, 520, 521, 522, 525, 526, 527, 530, 531, 532, 533, 535, 537, 538, 540, 541, 542, 543, 544, 545, 546, 547, 548, 549, 550, 551, 552, 553, 554, 556, 557, 558, 559, 560, 561, 562, 563, 564, 565, 567, 568, 569, 570, 571, 574, 575, 576, 580, 582, 583, 584, 585, 586, 587, 588, 589, 590, 591, 592, 593, 594, 595, 596, 597, 599, 600, 601, 602, 603, 604, 606, 607, 608, 611, 613, 618, 620, and 656, relative to SEQ ID NO: 5;
f) at least 70% identity to SEQ ID NO: 6 and one or more amino acid substitutions at positions: 1, 2, 3, 5, 6, 7, 9, 11, 12, 14, 21, 22, 26, 27, 31, 35, 38, 43, 44, 46, 47, 54, 59, 60, 61, 64, 65, 67, 68, 71, 72, 74, 76, 79, 80, 81, 84, 89, 95, 102, 105, 109, 110, 111, 112, 113, 114, 116, 118, 119, 120, 123, 129, 130, 131, 132, 134, 142, 145, 146, 147, 148, 150, 154, 155, 166, 169, 178, 180, 181, 183, 184, 187, 190, 194, 197, 201, 204, 207, 209, 213, 219, 221, 225, 226, 227, 229, 232, 233, 234, 236, 238, 241, 246, 251, 252, 256, 257, 261, 263, 265, 267, 269, 271, 272, 274, 280, 281, 285, 286, 288, 291, 292, 296, 299, 301, 303, 304, 306, 307, 308, 310, 313, 314, 316, 317, 318, 319, 320, 323, 324, 326, 328, 330, 331, 332, 340, 341, 343, 344, 355, 412, 418, 427, 514, 1198, 1201, 1206, 1212, 1260, and 1282, relative to SEQ ID NO: 6;
g) at least 70% identity to SEQ ID NO: 7 and one or more amino acid substitutions at positions: 99, 133, 189, 265, 266, 336, and 343, relative to SEQ ID NO: 7;
h) at least 70% identity to SEQ ID NO: 8 and one or more amino acid substitutions at positions: 119, 134, 155, 180, 183, 274, 319, 447, 454, 458, 461, 512, 538, and 580, relative to SEQ ID NO: 8;
i) at least 70% identity to SEQ ID NO: 9 and one or more amino acid substitutions at positions: 28, 82, 144, 151, 162, 182, 273, 327, 346, relative to SEQ ID NO: 9;
j) at least 70% identity to SEQ ID NO: 10 and one or more amino acid substitutions at positions: 21 and 90, relative to SEQ ID NO: 10;
k) at least 70% identity to SEQ ID NO: 11 and one or more amino acid substitutions at positions: 2, 3, 7, 9, 11, 12, 14, 16, 20, 26, 29, 32, 34, 35, 40, 43, 45, 46, 54, 61, 64, 65, 70, 77, 101, 103, 105, 106, 108, 109, 111, 119, 120, 123, 126, 127, 130, 131, 148, 149, 151, 157, 159, 164, 166, 185, 194, 196, 203, 211, 217, 218, 219, 236, 242, 257, 267, 279, 283, 286, 288, 291, 293, 296, 303, 306, 313, 314, 316, 326, 331, 336, 347, 352, 361, 374, 377, 395, 396, 398, and 408, relative to SEQ ID NO: 11;
l) at least 70% identity to SEQ ID NO: 12 and one or more amino acid substitutions at positions: 4, 5, 6, 8, 9, 11, 12, 13, 16, 17, 20, 21, 24, 26, 28, 29, 34, 37, 38, 41, 49, 54, 59, 60, 63, 65, 67, 74, 77, 81, 88, 92, 93, 94, 96, 102, 105, 106, 108, 110, 121, 126, 128, 134, 138, 142, 147, 150, 151, 153, 156, 157, 160, 162, 165, 170, 171, 173, 174, 179, 181, 183, 185, 186, 187, 188, 191, 198, 201, 206, 207, 226, 228, 233, 236, 241, 249, 250, 256, 267, 268, 270, 275, 276, 277, 279, 283, 286, 289, 303, 305, 306, 310, 312, 314, 315, 316, 323, 326, 329, 349, 353, 355, 356, 357, 358, 361, 370, 372, 373, 376, 378, 382, 388, 391, 397, 399, 403, 405, 419, 421, 423, 424, 425, 427, 428, 430, 431, 432, 433, 449, 457, 473, 477, 480, 485, 487, 489, 494, 496, 497, 498, 500, 502, 509, 511, 515, 518, 519, 520, 540, 545, 550, 555, 557, 570, 571, 580, 583, 585, 590, 594, 603, 607, 608, 611, 617, 620, 624, 636, 639, 641, 642, 644, 646, 655, 658, 660, 663, 665, 668, 672, 673, 678, 682, 685, 688, and 695, relative to SEQ ID NO: 12;
m) at least 70% identity to SEQ ID NO: 13 and one or more amino acid substitutions at positions: 5, 10, 11, 26, 30, 35, 40, 42, 45, 46, 47, 58, 61, 65, 71, 72, 75, 77, 78, 80, 82, 83, 94, 98, 113, 115, 116, 117, 121, 128, 133, 138, 146, 148, 161, 171, 175, 177, 182, 184, 191, 193, 201, 203, 211, 212, 219, 225, 226, 232, 233, 235, 236, 237, 238, 240, 250, 274, 282, 286, 292, 295, 304, 307, 309, 312, 313, 315, 316, 317, 318, 320, 321, 322, 323, 328, 340, 343, 344, 345, 347, 348, 349, and 350, relative to SEQ ID NO: 13; or
n) at least 70% identity to SEQ ID NO: 14 and one or more amino acid substitutions at positions: 2, 9, 13, 14, 15, 34, 38, 42, 46, 50, 59, 60, 73, 75, 77, 82, 83, 85, 86, 97, 110, 115, 120, 124, 130, 132, 134, 140, 143, 145, 156, 159, 162, 164, 177, 199, 232, and 270, relative to SEQ ID NO: 14.

3. A polypeptide of claim 1- or 2, comprising an amino acid sequence having:
a) at least 70% identity to SEQ ID NO: 1 and one or more amino acid substitutions of: A2T, T3I, L5S, T28A, A57T, F77L, Y80D, K107M, K107R, Y110C, Y110D, D116G, E122A, D142E, M155I, K161R, N166D, K173E, Y177N, Y177D, C185R, D211Y, K216E, A227P, G230D and G230S, relative to SEQ ID NO: 1;
b) at least 70% identity to SEQ ID NO: 2 and one or more amino acid substitutions of: A2T, A2S, G5R, S22P, E24D, L25I, A29S, P75T, I141T, V199I, S215R, D319V, Y347F, S364N, E370K, N383D, V439A, E454D, E454G, S458N, V485F, R509G, D533A, A538V, H565Y, A581T, H586L, N595K, D596N, D597N, D597Y, and I600V, relative to SEQ ID NO: 2;
c) at least 70% identity to SEQ ID NO: 3 and one or more amino acid substitutions of: 19V, A15V, F16Y, S18F, S21N, N64D, H81Y, D86Y, N87K, V99I, E109D, E142K, V147I, N153D, I168M, A180E, A216S, L230F, K285E, and R304R, relative to SEQ ID NO: 3;
d) at least 70% identity to SEQ ID NO: 4 and one or more amino acid substitutions of: R4K, N5K, P9S, A10P, N12D, T21I, V23M, S25N, S25R, V26M, V26G, S31N, S32I, E34A, F35L, A37D, H41L, D45N, I47V, E48G, G51V, S52I, E55K, E55D, E60K, F61L, S65T, S65A, P67T, P67L, P67S, P67H, T69A, A72V, A72D, S75I, S75R, S75T, K79E, T80P, K82E, K87R, P88L, P88T, P88A, S90F, K91N, K91E, A93T, A93S, S94N, L96P, R98Q, A99D, A99V, E100K, A103T, A106T, S108A, I113F, V116F, V116I, V125M, V125A, N126T, I128V, I128L, L129P, L135M, S139N, S139G, G143V, G143C, G146D, G146S, I147V, K149E, K149T, K149R, S153I, S153R, S153N, F154C, H156R, H156L, S158N, S158R, G159V, V160A, K162R, N164D, I166L, S167I, S168I, S168R, S168N, Q169R, V170M, V170G, V170L, T177I, T177A, S179R, F180C, F180L, F182C, F182L, G183S, M185I, K187R, G188D, V190I, K191N, A192S, D193N, G195V, G195D, G195S, C196W, T200A, T204I, A207V, A207T, and T208I, relative to SEQ ID NO: 4;
e) at least 70% identity to SEQ ID NO: 5 and one or more amino acid substitutions of: M1V, M1I, M1L, T2I, T2A, F4L, F5L, F8L, F8V, F8S, D9N, E10K, E10D, S11I, S11R, S11G, L12P, V13M, V13G, V13E, V13L, P14L, L15Q, K16N, K16R, P17T, P17L, P17S, T19I, T19S, T19A, T19P, P20S, P20L, T21A, Q22R, Y23H, V24M, K25R, L26M, D27A, D27G, D28N, D28Y, A29T, A29V, N30K, I32F, I32S, Q33H, L36M, D37A, D37Y, F39L, S40P, D41E, T42I, T42K, T42A, F43L, F43S, F43V, K44N, N45D, N45S, Q49R, K52Q, S55A, T56A, D58E, K60Q, S62T, R63K, R63G, Q67R, Q67H, Q67K, D71Y, K74R, E76K, F78C, K79R, G80V, G80D, G81S, G81V, G81D, D82N, V83G, V83M, V83A, V84A, V84G, R85G, R85K, P86L, N87S, R89C, V91G, V91A, A92V, A92T, R95K, K97R, E100D, S101A, D104V, A106D, A106T, D110N, N112H, H113Y, M115R, N117Y, T119A, N120D, N120K, N120S, G124V, D125N, D125E, K127R, F129L, D130N, K131M, E134D, E134G, A139S, A139T, P142S, I144V, A145S, A145T, T146A, A147V, Q149R, Y150H, I155L, V156A, V156L, V156M, K157V, E158A, N159S, V163G, E164A, E164G, E164D, G165D, I167V, I169L, I169T, N173S, N173H, N173T, A174S, A174T, N176D, A181S, I182L, I182V, I182T, A186E, A186T, V187G, V187A, A190T, A190S, F195S, A197P, D198G, D198N, A205S, V208M, P209T, T211I, E215D, E218D, P223S, P223H, L226V, I227V, D23IN, E232K, I235V, I235T, R239G, I246V, V248E, V248M, S250I, S259N, Y260C, K261R, S262N, P263L, S267N, A269V, T273I, T273N, H274Y, K277N, K277R, P278S, S280T, L281M, D282E, D282N, A283T, A283S, N285S, E287D, L288M, N290K, F295S, F298I, F298S, V302I, V303M, A307S, N313S, H316R, A317V, S320N, S320R, I323L, I325V, R331K, K332E, I339V, V345L, V345M, E348K, Y349H, Y349D, Y349N, Y349C, P352S, P352T, E353Q, E353D, L354M, G356S, N361D, I362V, I362T, L363P, L363T, L363M, E364G, K365R, E366G, E367G, K369N, K369E, K369M, P370S, E371K, V372M, D373G, I375V, M376I, T380P, T380A, E383K, E383D, F385L, H386Y, I389V, A390V, A390I, V392I, D396N, D396G, D396K, S397P, S399N, S399G, T402I, R403G, R403I, R403K, R403S, I404T, I404V, K407R, K407E, R408K, Q410K, Q410H, Q410R, Q411H, G412V, F413L, D414N, A415V, A415T, Y416C, M421I, N422K, E423K, E423D, E424A, E425K, E426D, T427A, T427S, R428K, F429L, S430A, M431L, R434H, R434C, R434S, I435V, D437G, D437N, T440S, T440I, R443C, G445S, F446L, F446I, Y448C, E450D, E450G, M452I, T456P, T456A, T456I, A459T, D460N, K463N, H464N, H464R, H464S, E470K, V472M, V472A, K473D, K473N, E494D, E494G, S495A, E498A, E498K, C501Y, T502I, T502S, P504S, P504L, T505A, G506Y, G506D, G506L, G506S, T508A, D509E, D509Y, C510Y, S512N, I513L, I513V, I513F, Y514H, K517M, K517N, K517Q, K520R, K521N, I522T, I522V, I522F, E525K, V526E, V526M, I527V, S530N, S530R, K531T, D532G, D532Y, S533Y, G535D, A537T, K538R, K538N, R540K, R540G, M541L, A542T, I543L, H544R, E545A, R546G, R546K, V547M, K548Q, K548R, Q549K, Q549R, E550A, Q551K, E552D, E552K, V553I, F554V, E556K, E556G, S557A, K558R, T559P, T559I, T559A, K560R, A561T, A561G, K562R, K562N, I563L, T564I, A565S, A565V, K567R, K568N, K568R, Q569K, Q569L, Q569R, A570V, Q571R, D574N, V575M, V575A, S576R, T580I, T580A, T582I, T582S, I583V, K584R, V585M, S586P, S586A, S586F, E587A, E588K, E588G, E588D, S589I, S589R, S589N, A590S, A590T, A591V, P592L, V593M, V593A, Q594L, K595R, K595N, H596Y, H596L, H596P, I597T, I597V, N599H, D600L, D600N, D600G, D600V, N601S, N601K, S602A, S602P, S602Y, D603A, D603V, D604G, D604Y, D604N, D606A, D606V, D606Y, D607Y, D607E, D608N, A611T, E613D, R618I, T620P, and A656V, relative to SEQ ID NO: 5;
f) at least 70% identity to SEQ ID NO: 6 and one or more amino acid substitutions of: M1L, M1V, N2S, A3T, T5P, T5A, T5S, E6D, 17S, 17V, 19F, Q11R, L12M, N14D, N14S, M21I, H22P, H22Y, K26N, K26R, T27I, M31I, L35R, N38S, S43P, D44N, D44G, Q46L, C47S, T54I, S59T, H60Y, T61A, H64Y, Y65H, K67N, K67R, R68Q, A71G, T72A, N74D, S76C, S76Y, T79I, M80I, P81S, V84L, R89L, A95D, A95T, A102T, E105D, E105K, S109N, S109R, S110P, Q111R, I112T, K113N, K113E, K114N, K114M, K114E, G116D, K118N, K118R, T119I, D120V, K123N, L129M, I130V, K131R, A132S, K134M, K134N, F142V, L145M, I146T, E147K, F148S, S150F, R154K, Q155H, E166D, K169E, P178S, A180V, A181T, A181S, I183V, A184S, A184T, A184V, P187S, A190T, A190V, V194M, V194A, R197I, Y201N, L204M, D207N, K209N, Q213H, Q213V, A219S, K221N, D225N, V226E, P227T, K229E, S232N, K233N, K233R, N234H, T236A, A238V, A238S, A241S, E246D, K251N, H252Y, H252R, E256D, A257S, A261V, S263I, S263N, N265D, Y267C, E269K, E269D, K271E, K271R, H272Y, I274V, F280L, D281N, D281G, K285G, K286N, K288R, S291F, S291P, K292N, K296R, K296N, I299S, D301G, E303D, I304T, I304V, E306G, V307L, V307G, V307A, V307D, V307G, I308N, N310S, Y313H, N314K, N316K, N316D, A317D, L318Q, D319N, P320S, P320L, M323I, L324M, D326N, V328M, V328A, A330D, I331V, V332G, S340L, T341A, A343G, S344N, I355V, F412V, V418F, Y427C, R514K, S1198L, A1201V, G1206S, C1212G, F1260L, and V1282M, relative to SEQ ID NO: 6;
g) at least 70% identity to SEQ ID NO: 7 and one or more amino acid substitutions of: M99I, S189N, H265Q, A266V, L336F, and V343A, relative to SEQ ID NO: 7;
h) at least 70% identity to SEQ ID NO: 8 and one or more amino acid substitutions of: Y119H, N134R, N134Q, D155N, Q180R, D183N, R274L, N319D, V447I, A454S, E458G, D461N, A512T, D538K, and P580Q, relative to SEQ ID NO: 8;
i) at least 70% identity to SEQ ID NO: 9 and one or more amino acid substitutions of: R28K, A82T, K144E, C151R, N162S, K182E, D273G, A327D, and M346I, relative to SEQ ID NO: 9;
j) at least 70% identity to SEQ ID NO: 10 and one or more amino acid substitutions of: A21S and V90A, relative to SEQ ID NO: 10;
k) at least 70% identity to SEQ ID NO: 11 and one or more amino acid substitutions of: A2T, F3S, P7R, A9S, A9G, A11G, F121, D14N, S16Y, Y20H, S26N, F29S, S32N, E34K, G35V, G35S, G35D, I40S, E43D, H45P, E46K, A54S, R61W, V64M, Y65C, N70S, A77T, D101N, K103E, N105K, N105D, S106G, V108M, A109G, Y111N, L119M, R120S, R123S, A126T, E127G, V130M, D131N, Q148R, S149Y, H151Y, A157D, T159I, A164V, L166M, T185A, S194G, A196T, T203A, K211R, E217K, R218K, R218S, N219S, A236T, E242D, N257K, N267S, M279I, M279V, D283G, N286S, T288I, K291Q, I293V, D296N, S303I, S303G, K306N, S310Y, S310P, I313T, Y314F, A316T, E326G, T331I, A336V, A347T, A347S, T352S, Y361H, M374T, M374I, R377G, T395I, S396T, S396F, G398V, and A408V, relative to SEQ ID NO: 11;
l) at least 70% identity to SEQ ID NO: 12 and one or more amino acid substitutions of: K4N, E5K, L6M, L6I, E8K, E8D, 19T, D11N, T12A, T13I, D16G, R17C, R17S, R20K, R20E, R21E, R21K, S24K, S24Q, S24R, Y26S, Y26H, A28S, A28D, M29I, G34D, A37S, V38M, V38G, I41V, R49L, D54G, K59R, K60N, K63N, A65T, A65V, K67E, K74E, K77E, W81C, K88R, K88E, 192T, R93E, R93K, V94M, K96N, E102D, E102G, T105A, L106M, S108P, V110A, G121S, S126P, K128R, L134M, Y138S, Q142H, W147L, K150N, V151M, V151L, A153T, S156R, S156G, D157N, K160R, K160E, A162T, S165N, S165G, V170E, K171E, F173V, K174N, K174R, T179A, K181T, S183N, P185T, E186K, E186D, E187K, A188S, A188V, D191Y, D191E, R198H, R198C, R198S, R201K, D206G, G207D, A226T, I228V, R233K, N236T, R241E, A249S, A250S, I256T, S267G, S267N, K268N, H270P, S275N, S275G, R276G, A277D, A277S, A277T, K279N, G283D, V286G, V289M, G303D, I305T, F306S, A310D, A310T, A312G, A312D, A312T, K314N, Q315R, R316G, N323S, E326A, E326K, N329S, G349D, E353D, L355M, L355R, E356G, E356D, S357P, A358V, R361S, P370T, N372K, E373D, S376F, T378I, F382L, M388V, G391S, R397K, A399S, K403N, M405I, L419P, D421N, K423R, H424N, H424R, V425L, I427V, E428K, D430A, D431G, E432D, H433N, A449T, G457D, R473K, E477D, G480D, F485L, S487R, S487G, S489N, N494D, S496N, A497S, V498G, K500N, K502N, Q509R, A511T, A511E, R515S, R518S, P519T, G520D, G520V, Y540C, Q545H, K550N, K555E, H557Q, P570S, E571D, C580R, S583R, E585K, E585G, E590D, R594K, M603I, H607N, H607L, K608R, D611N, L617P, N620S, K624N, T636P, M639V, N641S, V642G, S644N, S644G, E646D, A655V, V658M, K660N, T663A, T665I, R668S, I672V, G673V, S678R, M682L, A685V, A685D, K688N, and V695M, relative to SEQ ID NO: 12;
m) at least 70% identity to SEQ ID NO: 13 and one or more amino acid substitutions of: N5K, N5T, DION, R11K, D26N, V30E, D35N, R40L, P42A, G45S, G45V, F46V, T47R, T47S, N58T, P61L, T65I, T71I, T71R, T71D, L72M, C75S, V77A, P78L, N80T, E82D, H83Y, H83N, A94S, V98M, E113D, C121F, A128S, A155S, E116D, T117I, R133K, G138V, N146D, G148V, C161R, A171V, A171S, K175T, A177V, K182E, L184M, I191V, S193A, S193F, F201S, S203N, E211K, A212V, Y219R, N225S, N225T, D226Y, E232K, E232Q, A233N, A233S, A233K, K235R, Q236R, Q236S, F237L, V238Q, V238M, A240T, A240V, S250A, R274G, A282V, I286N, I286T, I286F, P292S, S295N, K304R, E307D, Y309C, A312V, L313M, N315K, N315T, N315S, C316G, I317V, T318A, T318P, K320R, N321D, E322K, K323N, I328T, M340I, K343E, K343R, K344E, K344R, A345T, A345D, A345S, A345Y, A345R, A345K, A345E, A345G, A347K, A347S, A347D, K348N, K349R, A350K, A350D, A350V, and A350T, relative to SEQ ID NO: 13; or
n) at least 70% identity to SEQ ID NO: 14 and one or more amino acid substitutions of: Q2K, H9L, K13E, Q14K, A15G, K34N, E38K, V42I, A46D, S50I, V59G, Y60H, A73S, A73T, F75L, D77G, G82S, F83L, F83V, F83C, K85E, V86I, E97, I110S, I110L, S115R, K120N, K124R, G130D, D132E, N134T, A140T, E143K, D145G, S156I, E159K, I162V, H164Y, H164F, Y177C, S199I, S232L, and L270S, relative to SEQ ID NO: 14.

4. A polypeptide of claim 1, comprising an amino acid sequence having:
a) at least 70% identity to SEQ ID NO: 1 and amino acid substitutions at positions: 2 and 230; 107 and 166; 107, 166, and one or both of: 2 and 227; 211 and 110 or 142; 110, 155 and 230; 122 and 155; or 155 and 177, relative to SEQ ID NO: 1;
b) at least 70% identity to SEQ ID NO: 2 and amino acid substitutions at positions: 2 and 597; 24 and 25; 24, 25, 458, 509, 565, and 600; 75 and 597; 141, 454, 533 and 595; 581, 370, and 454; 370 and 581; 370 and 454; 458 and 509; 458, 509 and 565; 458, 509, 565, and 600;
565, 586, and 596; or 565, 509, 458, 600 and at least one of 24, 25, 29, 215, 319, 364, 383, and 586, relative to SEQ ID NO: 2;
c) at least 70% identity to SEQ ID NO: 3 and amino acid substitutions at positions: 142 and 216, relative to SEQ ID NO: 3;
d) at least 70% identity to SEQ ID NO: 4 and amino acid substitutions at positions: 108 and 47 or 208; 170 and 207; 88 and 147; 47, 88 and 147; 88, 147, 170, and 182; 88, 147, 170, 182, and 51 or 180; 88, 147, and 154; 88, 128, 147, 170, and 182; or 170, 207, and 108, relative to SEQ ID NO: 4;
e) at least 70% identity to SEQ ID NO: 5 and amino acid substitutions at positions: 4, 23 and 590; 19, 169, and 549; 43 and 415; 80 and 593; 80, 144, 593, and 606; 1, 42, 80, 593, and 606; 42, 80, 593, and 606; 156 and 604; 283, 349, and 365; 283, 349, 365, 396, and 594; 283, 349, 365, 396, 594, 596, and 131; 352 and 390; 390, 396, and 594; 396 and 594; 456 and 502;
464 and 502; 464 and 17; 17, 235, 464, and 596; 235, 352, 396, 456, and 606; 415, 456, and 502;
456, 502, and 549; 169, 456, 502, and 549; 80, 456, 502, 593, and 606; 1, 42, 80, 456, 502, 593, and 606; 80, 144, 456, 502, 593, and 606; 19, 169, 456, 502 and 549; 43, 415, 456, and 502; 352, 390, 396, and 594; 352, 390, and 396; 283, 349, 396, and 594; 11, 55, 120, 362, 584, 600, and 604; 43, 84, 144, 349, and 517; 164 and 165; 164 and 173; 362 and 446; 352, 390, 396, 549, and 594; 352, 390, 396, 464, 549, and 594; 43, 349, 352, 390, 396, 464, 549, and 594; 43, 349, 352, 390, 396, 464, 549, 594 and one or more positions selected from 63, 145, 174, 182, 208, 410, 427, 456, 504, and 526; 43, 352, 390, 396, 464, 549, and 594; 43, 349, 352, 390, 396, 464, 549, 594, 410, 526, 415, and 502; 43, 349, 352, 390, 396, 464, 549, 594, 410, 526, 415, 502, and 21;
43, 349, 352, 390, 396, 464, 549, 594, 410, 526, 415, 502, and 67; 43, 349, 352, 390, 396, 464, 549, 594, 410, 526, 415, 502, 21, and 67; 43, 349, 352, 390, 396, 464, 549, 594, 410, 526, 174, 208, 427, 456, and 504; 43, 349, 352, 390, 396, 464, 549, 594, 410, 526, 415, 502, and 139; 43, 349, 352, 390, 396, 464, 549, 594, 410, 526, 415, 502, 339, and 446; 43, 349, 352, 390, 396, 464, 549, 594, 410, 526, 19, 460, 569, and 596; 43, 349, 352, 390, 396, 464, 549, 594, 410, 526, 460, 586, 588, and 608; 43, 349, 352, 390, 396, 464, 549, 594, 410, 526, and 460; 352, 390, 396, 549, 586, and 594; 63, 158, 352, 390, 396, 549, 586, and 594; 164, 165, 352, 363, 390, 396, 410, 549, 586, and 594; 164, 173, 352, 390, 396, 549, 586, and 594; 83, 352, 390, 396, 549, 586, and 594;
8, 43, 174, 349, 352, 390, 396, 427, 464, 549, and 594; or 283, 349, 365, 396, 594, 596, and 131, relative to SEQ ID NO: 5;
f) at least 70% identity to SEQ ID NO: 6 and amino acid substitutions at positions: 2, 67, 95, and 226; 6 and 316; 38, 95, and 303; 67, 95, and 226; 44 and 76; 44, 76, and 118; 130, 234, 303; 118 and 1201; 118, 1201, and 44; 118, 1201, and 76; 130, 234, and 303; 154 and 269; 221 and 44; 44, 76, 130, 234, and 303; 44, 76, 118, and 1201; 197 and 314; 76, 181, and 194; 76, 118, 252, and 292; 76 and 274; 76, 102, 118, and 307; 12 and 76; 67, 95, and 226; 26 and 76; 22, 76, 319; 154 and 269; 76 and 238; 76, 238, 296, and 328; 7 and 76; 76 and 263; 59, 76, 306, and 316; or 280 and 340, relative to SEQ ID NO: 6;
g) at least 70% identity to SEQ ID NO: 11 and amino acid substitutions at positions: 105, 109, 131, 148, 279, and 310; or 9, 105, 109, 131, 148, 279, and 310, relative to SEQ ID NO: 11;
h) at least 70% identity to SEQ ID NO: 12 and amino acid substitutions at positions: 134, 179, 185, 540, 555, 624, and 646; 138, 250, 275, and 421; 303, 405, 520, and 590; 134, 179, 185, 540, 555, and 646, 4 and 49; 4 and 388; 4 and 571; 4, 162, and 480; 4 and 315; 5 and 316; 17 and 156; 38, 108, 497 and 583; 59, 157 and 644; 96, 305, 550 and 642; 106, 160 and 228; 312, 424, 449 and 457; or 376 and 611, relative to SEQ ID NO: 12;
i) at least 70% identity to SEQ ID NO: 13 and amino acid substitutions at positions: 30, 46, 240, 304, and 316; 30, 46, 240, and 316; 42 and 318; 184, 240, 315, and 345; 211 and 274;
237 and 237; 286 and 350; 317 and 347; 171, 286, and 315; or 328 and 350, relative to SEQ ID NO: 13; or
j) at least 70% identity to SEQ ID NO: 14 and amino acid substitutions at positions: 82, 110, 115, 164, and 199; 82, 110, 115, 124, 164, and 199; 110, 115, and 164; 110, 115, 164, and 199; 110, 115, 164, 199, and 124; or 110, 115, 164, 199, and 82 or 124, relative to SEQ ID NO: 14.

5. A polypeptide of claim 1, comprising an amino acid sequence having:
a) at least 70% identity to SEQ ID NO: 1 and amino acid substitutions at positions: 155; 122 and 155; or 107, 166, and 227, relative to SEQ ID NO: 1;
b) at least 70% identity to SEQ ID NO: 2 and amino acid substitutions at positions: 24, 25, 458, 509, 565, and 600; 22, 347, and 454; or 485, relative to SEQ ID NO: 2;
c) at least 70% identity to SEQ ID NO: 4 and amino acid substitutions at positions: 75, 182; 88, 147, and 177; 88 and 147; 88, 116 and 147; 88, 147, 170, and 182; 88, 147, 170, 182, and 51 or 180; 88, 147, and 154; 75, 88, and 147; 47, 88 and 147; 88, 128, 147, 170, and 182; or 88, 93, and 147, relative to SEQ ID NO: 4;
d) at least 70% identity to SEQ ID NO: 5 and amino acid substitutions at positions: 352, 390, 396, 594, and 596; 352, 390, 396, 549, and 594; 352, 390, 396, 464, 549, and 594; 289, 352, 390, 396, 549, 594, and 596; 235, 352, 390, 396, 567, and 594; 352, 363, 390, 396, 549, 586, and 594; 352, 390, 396, 549, 580, and 594; 43, 349, 352, 390, 396, 464, 549, 594 and one or more positions selected from 63, 145, 174, 182, 208, 410, 427, 456, 504, and 526; 43, 349, 352, 390, 396, 464, 549, 594, 415 and 502; 43, 349, 352, 390, 396, 464, 549, 594, 415, 502 and 67; 43, 349, 352, 390, 396, 464, 549, 594, 415, 502 and 21; or 43, 349, 352, 390, 396, 464, 549, 594, 415, 502, 21 and 67; relative to SEQ ID NO: 5; or
e) at least 70% identity to SEQ ID NO: 6 and amino acid substitutions at positions: 197, 314, and optionally one of 7, 12, or 114; 197 and 314; 76, 181, and 194; 76, 118, 252, and 292; 76 and 274; 76, 102, 118, and 307; 12 and 76; 67, 95, and 226; 26 and 76; 22, 76, 319; 154 and 269; 76 and 238; 76, 238, 296, and 328; 7 and 76; 76 and 263; or 59, 76, 306, and 316, relative to SEQ ID NO: 6.

6. A polypeptide of claim 1, comprising an amino acid sequence having:
a) at least 70% identity to SEQ ID NO: 1 and amino acid substitutions: M155I; E122A and M155I; or K107M, N166D, and A227P, relative to SEQ ID NO: 1;
b) at least 70% identity to SEQ ID NO: 2 and amino acid substitutions at positions: E24D, L25I, S458N, R509G, H565Y, and I600V; S22P, Y347F, and E454G; or V485F, relative to SEQ ID NO: 2;
c) at least 70% identity to SEQ ID NO: 4 and amino acid substitutions: S75I; F182L; P88T, I147V, and T177I; P88T and I147V; P88T, V116I and I147V; P88T, I147V, V170L, and F182L; P88T, I147V, V170L, F180L, and F182L; G51V, P88T, I147V, V170L, and F182L; P88T, I147V, and F154C; S75I, P88T, and I147V; or P88T, A93T, and I147V, relative to SEQ ID NO: 4;
d) at least 70% identity to SEQ ID NO: 5 and amino acid substitutions: P352T, A390V, D396N, Q594L, and H596Y; P352S, A390V, D396N, Q549R, and Q594L; P352T, A390V, D396N, H464R, Q549R, and Q594L; Q289H, P352T, A390V, D396N, Q549R, Q594L, and H596Y; I235T, P352T, A390V, D396N, K567R, and Q594L; P352T, L363P, A390V, D396N, Q549R, S586A, and Q594L; P352T, A390V, D396N, Q549R, and Q594L; P352T, A390V, D396N, Q549R, T580I, and Q594L; F43S, Y349N or Y349D, P352T, A390V, D396N, H464R, Q549R, Q594L and one or more substitutions selected from R63G, A145S, A174S, I182R, V208M, Q410K, T427S, T456I or T456P, P504S, and V526E; F43S, Y349N or Y349D, P352T, A390V, D396N, H464R, Q549R, Q594L, A415V and T502I; F43S, Y349N or Y349D, P352T, A390V, D396N, H464R, Q549R, Q594L, A415V, T502I, and T21A; F43S, Y349N or Y349D, P352T, A390V, D396N, H464R, Q549R, Q594L, A415V, T502I, and Q67K; or F43S, Y349N or Y349D, P352T, A390V, D396N, H464R, Q549R, Q594L, A415V, T502I, T21A, and Q67K; relative to SEQ ID NO: 5; or
e) at least 70% identity to SEQ ID NO: 6 and amino acid substitutions at positions: R197I, N314K, and optionally one of I7S, L12M, or K114M; R197I and N314K; S76Y, A181S, and V194M; S76Y, K118R, H252R, and K292N; S76Y and I274V; S76Y, A102T, K118R, and V307G; L12M and S76Y; K67N, A95D, and V226E; K26N and S76Y; H22Y, S76Y, and D319N; R154K and E269D; S76Y and A238S; S76Y, A238S, K296N, and V328M; I7V and S76Y; S76Y and S263N; or S59T, S76Y, E306G, and N316D, relative to SEQ ID NO: 6.

7. The polypeptide of claim 1, comprising an amino acid sequence having at least 70% identity to SEQ ID NO: 13 and at least one amino acid substitution with a positively charged amino acid, optionally selected from arginine or lysine.

8. The polypeptide of claim 7, wherein the at least one amino acid substitution is at position 2, 5, 6, 7, 8, 9, 10, 12, 38, 39, 40, 41, 42, 43, 44, 45, 46, 47, 48, 64, 65, 66, 67, 68, 69, 70, 222, 224, 225, 227, 228, 229, 231, 232, 233, 234, 235, 255, 256, 257, 258, 277, 286, 287, 337, 338, 339, 340, 345, 346, 347, 348, 349, 350, or a combination thereof, relative to SEQ ID NO: 13.

9. The polypeptide of claim 7, wherein the at least one amino acid substitution is at positions: 346 and 348; 346, 348 and 349; 346, 348, 349, an 350; 350 and 351; 350, 351, and 352; 350, 351, 352, and 353; 235 and 227; 235 and 345; 235 and 346; 235 and 347; 235 and 348; 235 and 349; 235 and 350; 235, 227, and 349; 5, 235 and 346; 5, 235 and 348; 5, 235 and 349; 227, 235, and 346; or 227, 235, and 348, relative to SEQ ID NO: 13.

10. The polypeptide of claim 1, comprising:
a first amino acid sequence having at least 70% identity to SEQ ID NO: 1 with one or more amino acid substitutions, deletions, or additions relative to SEQ ID NO: 1, and
a second amino acid sequence having at least 70% identity to SEQ ID NO: 2 with one or more amino acid substitutions, deletions, or additions relative to SEQ ID NO: 2; or
a first amino acid sequence having at least 70% identity to SEQ ID NO: 4 with one or more amino acid substitutions, deletions, or additions relative to SEQ ID NO: 4, and
a second amino acid sequence having at least 70% identity to SEQ ID NO: 5 with one or more amino acid substitutions, deletions, or additions relative to SEQ ID NO: 5.

11. A composition comprising one or more polypeptides of claim 1, or one or more nucleic acids encoding thereof, and optionally one or more Cas proteins or one or more nucleic acids encoding thereof and/or at least one unfoldase protein or at least one nucleic acid encoding thereof.

12. A system comprising
an engineered Clustered Regularly Interspaced Short Palindromic Repeats (CRISPR)-associated transposon (CRISPR-Tn) system or one or more nucleic acids encoding the engineered CRISPR-Tn system, wherein the CRISPR-Tn system comprises at least one or both of:
a) one or more Cas proteins selected from: Cas5, Cas6, Cas7, Cas8, Cas9 and combinations thereof; and
b) one or more transposon-associated proteins selected from TnsA, TnsB, TnsC, TnsD, TniQ, and combinations thereof,
wherein at least one of the one or more Cas protein or at least one of the one of the one or more transposon-associated proteins comprises a polypeptide of claim 1.

13. The system of claim 12, further comprising:
at least one guide RNA (gRNA) complementary to at least a portion of a target nucleic acid, or at least one nucleic acid encoding thereof;
a donor nucleic acid, wherein the donor nucleic acid comprises a cargo nucleic acid sequence flanked by at least one transposon end sequence;
at least one unfoldase protein, or at least one nucleic acid encoding thereof;
a target nucleic acid; or
a combination thereof.

14. A method for nucleic acid modification or integration comprising contacting a target nucleic acid sequence or a cell comprising a target nucleic acid with a polypeptide of claim 1 or a system comprising thereof.

15. A cell comprising a polypeptide of claim 1 or a nucleic acid encoding thereof.

16. A polypeptide of claim 1, comprising one or more amino acid sequences having at least 80% identity to any of SEQ ID NOs: 1-14 with one or more amino acid substitutions, deletions, or additions relative to SEQ ID NOs: 1-14.

17. A polypeptide of claim 1, comprising one or more amino acid sequences having at least 90% identity to any of SEQ ID NOs: 1-14 with one or more amino acid substitutions, deletions, or additions relative to SEQ ID NOs: 1-14.
